(19) United States
(12) Patent Application Publication
(10) Pub. No.: US 2024/0090321 A1
(43) Pub. Date: Mar. 14, 2024

(54) ORGANIC ELECTROLUMINESCENT COMPOUND AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: So-Young JUNG, Gyeonggi-do (KR); Hyun-Ju KANG, Gyeonggi-do (KR); Sang-Hee CHO, Gyeonggi-do (KR); Tae-Jun HAN, Gyeonggi-do (KR); Hyo-Nim SHIN, Gyeonggi-do (KR); Eun-Joung CHOI, Gyeonggi-do (KR)

(21) Appl. No.: 18/353,356

(22) Filed: Jul. 17, 2023

(30) Foreign Application Priority Data
Aug. 3, 2022 (KR) 10-2022-0096762

(51) Int. Cl. H10K 85/60; C07C 211/61; C09K 11/06

(52) U.S. Cl. CPC H10K 85/633; C07C 211/61; C09K 11/06; H10K 50/11

(57) ABSTRACT

The present disclosure relates to an organic electroluminescent compound and an organic electroluminescent device comprising the same. By comprising the compound according to the present disclosure, an organic electroluminescent device having improved driving voltage, power efficiency and/or lifetime characteristics compared to conventional organic electroluminescent devices can be provided.

### TECHNICAL FIELD

The present disclosure relates to an organic electroluminescent compound and an organic electroluminescent device comprising the same.

### BACKGROUND ART

An electroluminescent device (EL device) is a self-light-emitting display device which has advantages in that it provides a wider viewing angle, a greater contrast ratio, and a faster response time. The first organic EL device was developed by Eastman Kodak in 1987, by using small aromatic diamine molecules and aluminum complexes as materials for forming a light-emitting layer [Appl. Phys. Lett. 51, 913, 1987].

An organic electroluminescent device (OLED) changes electric energy into light by applying electricity to an organic light-emitting material, and commonly comprises an anode, a cathode, and an organic layer formed between the two electrodes. The organic layer of the OLED may comprise a hole injection layer, a hole transport layer, a hole auxiliary layer, a light-emitting auxiliary layer, an electron blocking layer, a light-emitting layer, an electron buffer layer, a hole blocking layer, an electron transport layer, an electron injection layer, etc., if necessary. The materials used in the organic layer can be classified into a hole injection material, a hole transport material, a hole auxiliary material, a light-emitting auxiliary material, an electron blocking material, a light-emitting material (containing host and dopant materials), an electron buffer material, a hole blocking material, an electron transport material, an electron injection material, etc., depending on functions. In the OLED, holes from an anode and electrons from a cathode are injected into a light-emitting layer by the application of electric voltage, and an exciton having high energy is produced by the recombination of the holes and electrons. The organic light-emitting compound moves into an excited state by the energy and emits light from energy when the organic light-emitting compound returns to the ground state from the excited state.

Selection of compounds included in a hole transport layer, etc., is recognized as a means that can improve device characteristics such as hole transport efficiency to a light-emitting layer, luminous efficiency, and lifetime. Recently, an urgent task is the development of an OELD having high efficiency and long lifetime.

In particular, the development of highly excellent light-emitting material over conventional materials is urgently required, considering the EL properties necessary for medium- and large-sized OLED panels.

On the other hand, International Publication No. 2019/235725 discloses a compound having the structure in which an amino group is attached to a naphthocarbazole derivative. In addition, Korean Patent Application Laid-open No. 2012-0116881 discloses a compound having the structure in which two amino groups are attached to a naphtofluorene derivative. However, the aforementioned references fail to specifically disclose that a compound having the structure in which one amino group is attached to a fluorene fused with naphthalene can improve the performance of an OLED.

### DISCLOSURE OF INVENTION

### Technical Problem

An objective of the present disclosure is to provide an organic electroluminescent compound effective for producing an organic electroluminescent device with low driving voltage, high power efficiency, and/or improved lifetime properties.

### Solution to Problem

The present inventors found that the above objective can be achieved by an organic electroluminescent compound represented by the following formula 1.

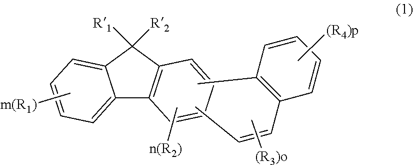

In formula 1,
R′₁ and R′₂, each independently, represent a substituted or unsubstituted (C1-C30)alkyl, a substituted or unsubstituted (C6-C30)aryl, or a substituted or unsubstituted (3- to 30-membered)heteroaryl; or R′₁ and R′₂ may be linked to each other to form a ring(s); and R′₁ and R′₂ may be the same as or different from each other;
R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L₁-N(Ar₁)-L₂-N—(Ar₂)(Ar₃); or may be linked to an adjacent substituent(s) to form a ring(s);
with a proviso that any one of R₁'s to R₄'s represents -L₁-N(Ar₁)-L₂-N—(Ar₂)(Ar₃);
L₁ represents a single bond, a substituted or unsubstituted (C6-C30)arylene, or a substituted or unsubstituted (3- to 30-membered)heteroarylene;
L₂ represents a substituted or unsubstituted (C6-C30)arylene, or a substituted or unsubstituted (3- to 30-membered)heteroarylene;
Ar₁ to Ar₃, each independently, represent a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl; and
m and p, each independently, represent an integer of 4; n and o, each independently, represent an integer of 2; and each of R₁ to each of R₄ may be the same as or different from each other.

### Advantageous Effects of Invention

An organic electroluminescent compound according to the present disclosure can provide an organic electroluminescent device having low driving voltage, high power efficiency and/or improved lifetime properties.

### MODE FOR THE INVENTION

Hereinafter, the present disclosure will be described in detail. However, the following description is intended to explain the present disclosure, and is not meant to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

Herein, the term “(C1-C30)alkyl” is meant to be a linear or branched alkyl having 1 to 30 carbon atoms constituting the chain, in which the number of carbon atoms is preferably 1 to 10, and more preferably 1 to 6. The above alkyl may include methyl, ethyl, n-propyl, isopropyl, n-butyl, isobutyl, tert-butyl, sec-butyl, etc. The term “(C3-C30)cycloalkyl” is meant to be a monocyclic or polycyclic hydrocarbon having 3 to 30 ring backbone carbon atoms, and preferably 3 to 20 ring backbone carbon atoms, more preferably 3 to 7 ring backbone carbon atoms. Examples of the cycloalkyl may include cyclopropyl, cyclobutyl, cyclopentyl, cyclohexyl, cyclopentylmethyl, cyclohexylmethyl, etc. The term “(3- to 7-membered)heterocycloalkyl” in the present disclosure is meant to be a cycloalkyl having 3 to 7 ring backbone atoms and containing at least one heteroatom(s) selected from the group consisting of B, N, O, S, Si, P, Te, and Se, preferably the group consisting of O, S, N, Te, and Se. The above heterocycloalkyl includes tetrahydrofuran, pyrrolidine, thiolane, tetrahydropyran, etc. The term “(C6-C30)aryl” or “(C6-C30)arylene” in the present disclosure is meant to be a monocyclic or fused ring radical derived from an aromatic hydrocarbon having 6 to 30 ring backbone carbon atoms. The above aryl may be partially saturated, and may comprise a spiro structure. The above aryl may include phenyl, biphenyl, terphenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, benzophenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluorene]yl, spiro[cyclopentene-fluorene]yl, spiro[dihydroindene-fluorene]yl, azulenyl, tetramethyldihydrophenanthrenyl, etc. More specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl” or “(3- to 30-membered)heteroarylene” in the present disclosure is meant to be an aryl or arylene having 3 to 30 ring backbone atoms and including at least one, preferably 1 to 4 heteroatom(s) selected from the group consisting of B, N, O, S, Si, P, Te, and Se. It may be a monocyclic ring or a fused ring condensed with at least one benzene ring, and may be partially saturated. In addition, the above heteroaryl or heteroarylene comprises one formed by linking at least one heteroaryl or aryl group to a heteroaryl group via a single bond(s), and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, benzofuroquinolyl, benzofuroquinazolinyl, benzofuronaphthyridinyl, benzofuropyrimidinyl, naphthofuropyrimidinyl, benzothienoquinolyl, benzothienoquinazolinyl, naphthyridinyl, benzothienonaphthyridinyl, benzothienopyrimidinyl, naphthothienopyrimidinyl, pyrimidoindolyl, benzopyrimidoindolyl, benzofuropyrazinyl, naphtofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzooxazolyl, isoindolyl, indolyl, indazolyl, benzothiadiazolyl, quinolyl, isoquinolyl, cinnolinyl, quinazolinyl, benzoquinazolinyl, quinoxalinyl, benzoquinoxalinyl, carbazolyl, benzocarbazolyl, dibenzocarbazolyl, phenoxazinyl, phenanthridinyl, benzodioxolyl, dihydroacridinyl, benzotriazolyl, phenazinyl, imidazopyridyl, chromenoquinazolinyl, thiochromenoquinazolinyl, dimethylbenzoperimidinyl, indolocarbazolyl, indenocarbazolyl, etc. More specifically, the heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. In the present disclosure, “halogen” includes F, Cl, Br, and I.

In addition, “ortho (o-),” “meta (m-),” and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

In addition, “substituted” in the expression “substituted or unsubstituted” means that a hydrogen atom in a certain functional group is replaced with another atom or another functional group (i.e., a substituent), and also includes that the hydrogen atom is replaced with a group formed by a linkage of two or more substituents of the above substituents. For example, the “group formed by a linkage of two or more substituents” may be pyridine-triazine. That is, pyridine-triazine may be interpreted as a heteroaryl substituent, or as substituents in which two heteroaryls are linked. Herein, the substituent(s) of the substituted alkyl, the substituted alkenyl, the substituted alkynyl, the substituted aryl, the substituted arylene, the substituted heteroaryl, the substituted heteroarylene, the substituted cycloalkyl, the substituted alkoxy, the substituted trialkylsilyl, the substituted dialkylarylsilyl, the substituted alkyldiarylsilyl, the substituted triarylsilyl, the substituted fused ring group of an aliphatic ring(s) and an aromatic ring(s), the substituted alkylarylamino, the substituted mono- or di- alkylamino, the substituted mono- or di- arylamino, the substituted mono- or di- heteroarylamino, and the substituted arylheteroarylamino, each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl unsubstituted or substituted with a (C6-C30)aryl(s); a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium and a (C6-C30)aryl(s); a (C6-C30)aryl unsubstituted or substituted with at least one of deuterium, a halogen(s), a cyano(s), a (C1-C30)alkyl(s), a (C6-C30)aryl(s), a (3- to 30-membered)heteroaryl(s), a tri(C6-C30)arylsilyl(s), and a tri(C6-C30)arylgermanyl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a tri(C1-C30)alkylgermanyl; a tri(C6-C30)arylgermanyl; a di(C1-C30)alkyl(C6-C30)arylgermanyl; a (C1-C30)alkyldi(C6-C30)arylgermanyl; a fused ring group of a (C3-C30) aliphatic ring(s) and a (C6-C30) aromatic ring(s); an amino; a mono- or di- (C1-C30)alkylamino; a mono- or di- (C2-C30)alkenylamino; a mono- or di- (C6-C30)arylamino unsubstituted or substituted with a (C1-C30)alkyl(s); a mono- or di- (3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphinyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituent(s), each independently, are at least one selected from the group consisting of deuterium; a halogen; a cyano; a (C1-C20)alkyl; a (C2-C20)alkenyl unsubstituted or substituted with a (C6-C25)aryl(s); a (C6-C25)aryl unsubstituted or substituted with at least one of deuterium, a halogen(s), a cyano(s), a (C1-C20)alkyl(s), a (C6-C25)aryl(s), a (5- to 20-membered)heteroaryl(s), a tri(C6-C18)arylsilyl(s), and a tri(C6-C18)arylgermanyl(s); a (5- to 25-membered)heteroaryl unsubstituted or substituted with at least one of deuterium and a (C6-C25)aryl(s); a tri(C6-C18)arylsilyl; a mono- or di- (C6-C25)arylamino; a mono- or di- (5- to 25-membered)heteroarylamino; a (C6-C25)aryl(5- to 25-membered)heteroarylamino; a tri(C6-C18)arylgermanyl; and a (C6-C25)aryl(C1-C20)alkyl. According to another embodiment of the present disclosure, the substituent(s), each independently, are at least one selected from the group consisting of deuterium; a cyano; a (C1-C10)alkyl; a (C2-C10)alkenyl unsubstituted or substituted with a (C6-C18)aryl(s); a (C6-C25)aryl unsubstituted or substituted with at least one of deuterium, a halogen(s), a cyano(s), a (C1-C10)alkyl(s), a (C6-C18)aryl(s), a (5- to 20-membered)heteroaryl(s), and a tri(C6-C18)arylgermanyl(s); a (5- to 20-membered)heteroaryl unsubstituted or substituted with at least one of deuterium and a (C6-C18)aryl(s); a tri(C6-C18)arylsilyl; a di(C6-C18)arylamino; a di(5- to 20-membered)heteroarylamino; a (C6-C18)aryl(5- to 20-membered)heteroarylamino; a tri(C6-C18)arylgermanyl; and a (C6-C18)aryl(C1-C10)alkyl. For example, the substituent(s), each independently, may be any one selected from the group consisting of deuterium; a cyano; a methyl; a tert-butyl; an ethylene substituted with a phenyl(s); a substituted or unsubstituted phenyl; a substituted or unsubstituted naphthyl; a biphenyl; a phenanthrenyl; a terphenyl; a chrysenyl; a benzo[c]phenanthryl; a triphenylenyl; a dimethylfluorenyl; a diphenylfluorenyl; a dimethylbenzofluorenyl; a spirobifluorenyl; a pyridyl; a pyrimidinyl; a triazinyl substituted with a phenyl(s); an indolyl; a dibenzofuranyl unsubstituted or substituted with at least one of deuterium and a phenyl(s); a dibenzotiophenyl; a carbazolyl unsubstituted or substituted with at least one of a phenyl(s) and a biphenyl(s); a phenylpropyl; a dibenzotelluriumyl; a dibenzoselenophenyl; a benzonaphthofuranyl; a benzonaphthothiophenyl; a phenanthrooxazolyl substituted with a phenyl(s); a triphenylsilyl; a diphenylamino; a phenyldibenzofuranylamino; and a triphenylgermanyl, or a combination thereof, in which the substituent(s) of the substituted phenyl may be at least one of deuterium, a cyano, a fluoro, a methyl, a naphthyl, a carbazolyl, a triphenylsilyl, a triphenylgermanyl, and a dibenzotelluriumyl, and the substituent(s) of the substituted naphthyl may be at least one of a phenyl, a biphenyl, and a chrysenyl.

In the present disclosure, “a ring formed by a linkage of adjacent substituents” means that at least two adjacent substituents are linked or fused to each other to form a substituted or unsubstituted, mono- or polycyclic, (3- to 30-membered) alicyclic or aromatic ring, or the combination thereof. The ring may be preferably a substituted or unsubstituted, mono- or polycyclic, (3- to 26-membered) alicyclic or aromatic ring, or the combination thereof, and more preferably a mono- or polycyclic, (5- to 25-membered) aromatic ring unsubstituted or substituted with at least one of a (C1-C6) akyl(s), a (C6-C18)aryl(s) and a (3- to 20-membered)heteroaryl(s). In addition, the formed ring may contain at least one heteroatom selected from the group consisting of B, N, O, S, Si, and P, preferably at least one heteroatom selected from the group consisting of N, O, and S. For example, the ring may be a benzene ring, a cyclopentane ring, an indene ring, an indan ring, a fluorene ring, a phenanthrene ring, an indole ring, a benzofuran ring unsubstituted or substituted with a phenyl(s), a benzothiophene ring unsubstituted or substituted with a phenyl(s), a xanthene ring, etc., and the ring may comprise a spiro ring.

In the present disclosure, a heteroaryl, a heteroarylene, and a heterocycloalkyl, each independently, may include at least one heteroatom(s) selected from the group consisting of B, N, O, S, Si, P, Te, and Se. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C2-C30)alkenylamino, a substituted or unsubstituted mono- or di-(C6-C30)arylamino, a substituted or unsubstituted mono- or di- (3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, and a substituted or unsubstituted (C6-C30)aryl(3- to 30-membered)heteroarylamino.

In formula 1, R′₁ and R′₂, each independently, represent a substituted or unsubstituted (C1-C30)alkyl, a substituted or unsubstituted (C6-C30)aryl, or a substituted or unsubstituted (3- to 30-membered)heteroaryl; or R′₁ and R′₂ may be linked to each other to form a ring(s). R′₁ and R′₂ may be the same as or different from each other. According to one embodiment of the present disclosure, R′₁ and R′₂, each independently, represent a substituted or unsubstituted (C1-C20)alkyl, a substituted or unsubstituted (C6-C25)aryl, or a substituted or unsubstituted (5- to 25-membered)heteroaryl; or R′₁ and R′₂ may be linked to each other to form a ring(s). According to another embodiment of the present disclosure, R′₁ and R′₂, each independently, represent an unsubstituted (C1-C10)alkyl, an unsubstituted (C6-C18)aryl, or an unsubstituted (5- to 20-membered)heteroaryl; or R′₁ and R′₂ may be linked to each other to form a spiro ring(s), which may be substituted with dueterium. For example, R′₁ and R′₂, each independently, may be a methyl, an ethyl, a propyl, a phenyl, a naphthyl, a pyridyl, etc., which are unsubstituted or substituted with deuterium; or R′₁ and R′₂ may be linked to each other to form a spirofluorene ring(s) or a spiroindan ring(s), etc.

In formula 1, m and p, each independently, represent an integer of 4; n and o, each independently, represent an integer of 2; and each of R₁ to each of R₄ may be the same as or different from each other.

In formula 1, R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L₁-N(Ar₁)-L₂-N—(Ar₂)(Ar₃); or may be linked to an adjacent substituent(s) to form a ring(s). However, any one of R₁'s to R₄'s represents -L₁-N(Ar₁)-L₂-N—(Ar₂)(Ar₃). According to one embodiment of the present disclosure, R₁ to R₄, each independently, represent hydrogen, deuterium, a (C1-C10)alkyl unsubstituted or substituted with deuterium, or -L₁-N(Ar₁)-L₂-N—(Ar₂)(Ar₃). For example, R₁ to R₄, each independently, may be hydrogen, deuterium, a methyl, or -L₁-N(Ar₁)-L₂-N—(Ar₂)(Ar₃).

L₁ represents a single bond, a substituted or unsubstituted (C6-C30)arylene, or a substituted or unsubstituted (3- to 30-membered)heteroarylene. For example, L₁ may be a single bond, a phenylene unsubstituted or substituted with a phenyl(s), a naphthylene, a biphenylene, a dibenzothiophenylene, a dibenzofuranylene, etc., which may be substituted with deuterium.

L₂ represents a substituted or unsubstituted (C6-C30)arylene, or a substituted or unsubstituted (3- to 30-membered)heteroarylene. According to one embodiment of the present disclosure, L₂ represents a substituted or unsubstituted (C6-C25)arylene, or a substituted or unsubstituted (5- to 25-membered)heteroarylene. According to another embodiment of the present disclosure, L₂ represents a (C6-C18)arylene unsubstituted or substituted with at least one of a (C6-C18)aryl(s), a mono- or di- (C6-C30)arylamino(s), a mono- or di- (3- to 30-membered)heteroarylamino(s), and a (C6-C30)aryl(3- to 30-membered)heteroarylamino(s); or an unsubstituted (5- to 20-membered)heteroarylene, which may be further substituted with deuterium. For example, L₂ may be a phenylene unsubstituted or substituted with at least one of a phenyl(s) and a diphenylamino(s), a naphthylene, a biphenylene, a dibenzothiophenylene, dibenzofuranylene, etc., which may be further substituted with deuterium.

Ar₁ to Ar₃, each independently, represent a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl. Ar₁ to Ar₃ may be the same as or different from one another. According to one embodiment of the present disclosure, Ar₁ to Ar₃, each independently, represent a substituted or unsubstituted (C6-C25)aryl, or a substituted or unsubstituted (5- to 25-membered)heteroaryl. According to another embodiment of the present disclosure, Ar₁ to Ar₃, each independently, represent a substituted or unsubstituted (C6-C24)aryl, or a (5- to 20-membered)heteroaryl unsubstituted or substituted with a (C6-C18)aryl(s), in which the substituent(s) of the substituted aryl may be any one of deuterium, a (C1-C10)alkyl, a (C2-C10)alkenyl, a (C6-C18)aryl, a (5- to 15-membered)heteroaryl, a di(C6-C18)arylamino, a (C6-C18)aryl(5- to 15-membered)heteroarylamino, and a (C6-C18)aryl(C1-C10)alkyl, or a combination thereof. For example, Ar₁ to Ar₃, each independently, may be a substituted or unsubstituted phenyl, a naphthyl, a phenylnaphthyl, a naphthylphenyl, a biphenyl, a phenanthrenyl, a terphenyl, a quaterphenyl, a triphenylenyl, a chrysenyl, a dimethylfluorenyl, a diphenylfluorenyl, a benzofuranyl, a benzothiophenyl, a dibenzofuranyl unsubstituted or substituted with a phenyl(s), a dibenzothiophenyl unsubstituted or substituted with a phenyl(s), a dibenzoselenophenyl unsubstituted or substituted with a phenyl(s), a phenylcarbazolyl, a phenoxazinyl substituted with a phenyl(s), etc., which may be further substituted with deuterium, in which the substituent(s) of the substituted phenyl may be at least one selected from the group consisting of deuterium, a methyl, a tert-butyl, a triphenylethylenyl, a dimethylfluorenyl, a dibenzofuranyl, a dibenzothiophenyl, an indolyl, a pyridyl, a pyrimidinyl, a carbazolyl, a diphenylamino, a phenyldibenzofuranylamino, and a phenylpropyl.

Formula 1 may be represented by the following formula 1-1.

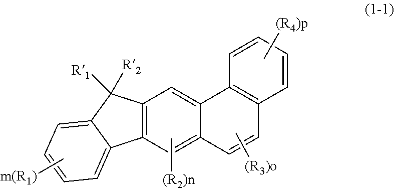

In formula 1-1, R′₁, R′₂, R₁ to R₄, and m to p are as defined in formula 1.

Formula 1 may be represented by any one of the following formulas 1-1-1 to 1-1-4.

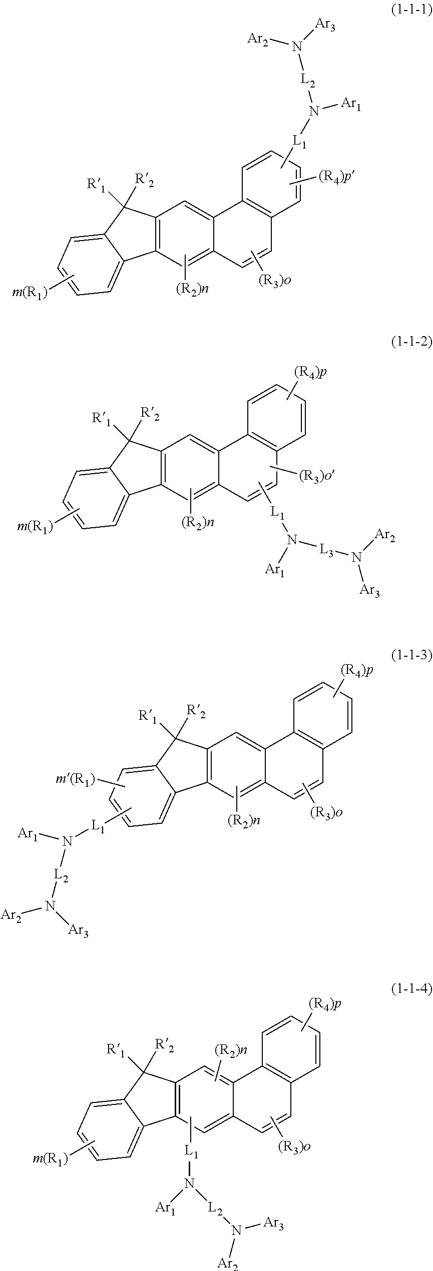

In formulas 1-1-1 to 1-1-4, R′₁, R′₂, R₁ to R₄, L₁, L₂, Ar₁ to Ar₃, and m to p are as defined in formula 1.

In formulas 1-1-1 to 1-1-4, m′ and p′, each independently, represent an integer of 3, and n‘ and o’, each independently, represent an integer of 1.

In formulas 1-1-1 to 1-1-4, R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s); or may be linked to an adjacent substituent(s) to form a ring(s). For example, R₁ to R₄ may be hydrogen.

Formula 1 may be represented by any one of the following formulas 1-1-1-1 to 1-1-1-12.

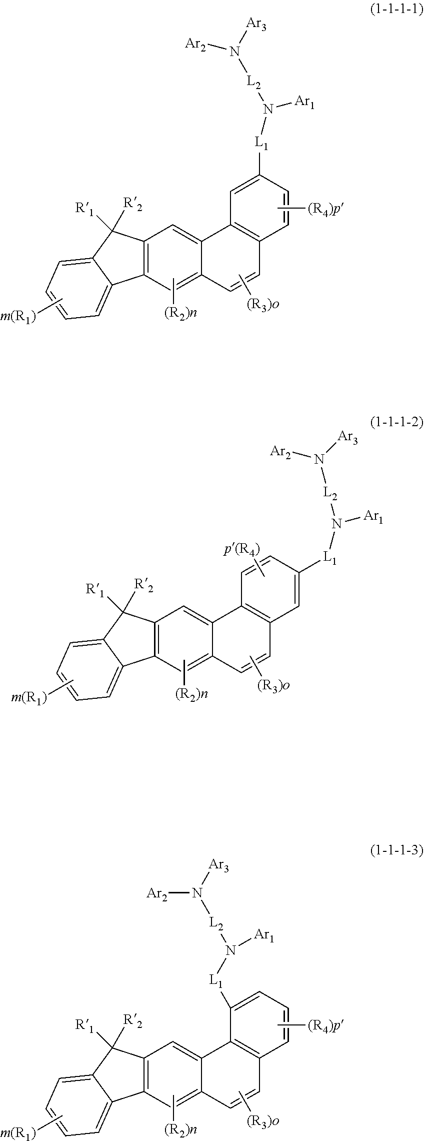

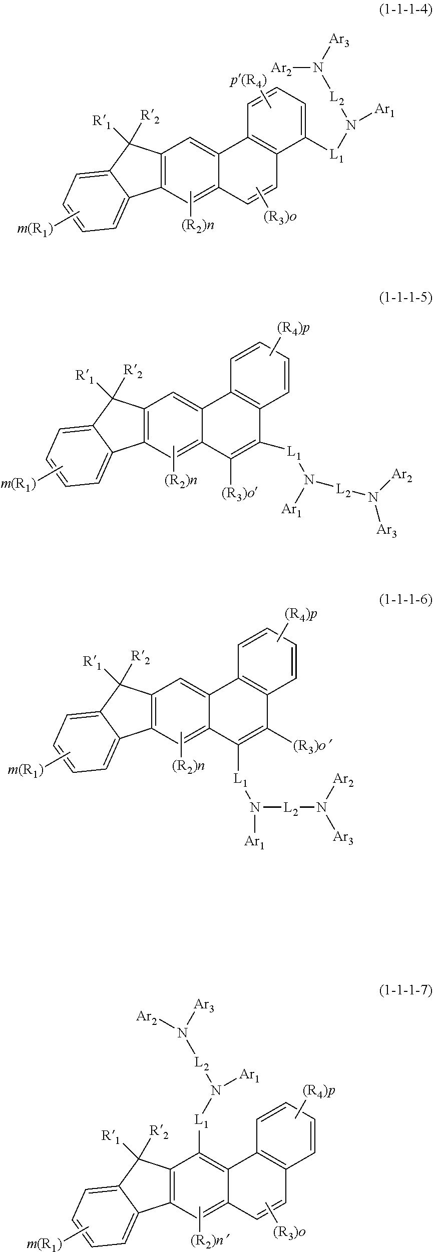

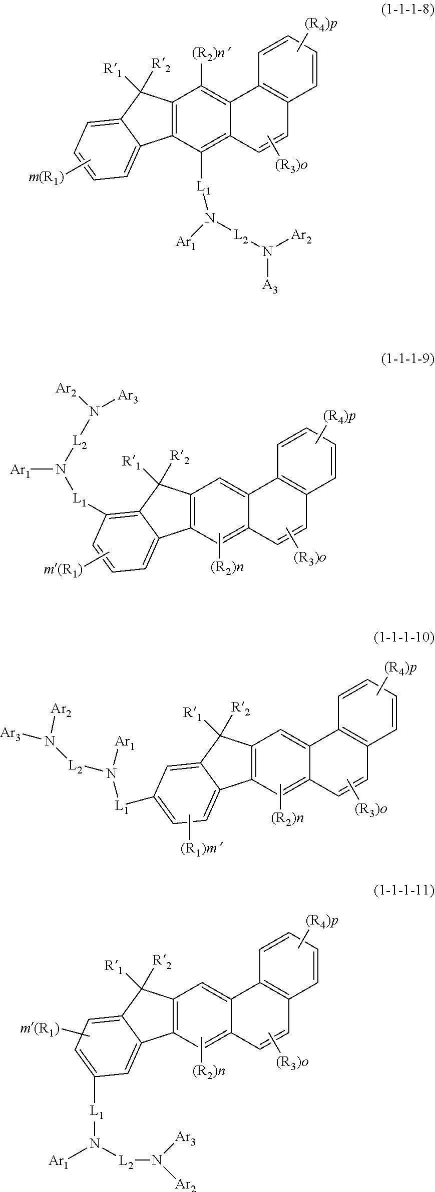

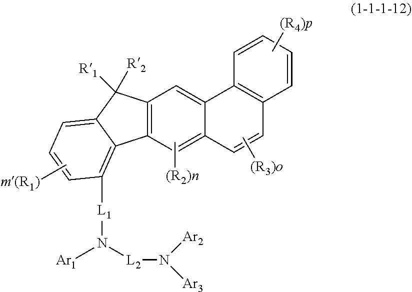

In formulas 1-1-1-1 to 1-1-1-12, R′₁, R′₂, R₁ to R₄, L₁, L₂, Ar₁ to Ar₃, and m to p are as defined in formula 1.

In formulas 1-1-1-1 to 1-1-1-12, m′ and p′, each independently, represent an integer of 3, and n′ and o′, each independently, represent an integer of 1.

In formulas 1-1-1-1 to 1-1-1-12, R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s); or may be linked to an adjacent substituent(s) to form a ring(s). For example, R₁ to R₄ may be hydrogen.

The compound represented by formula 1 may be at least one selected from the group consisting of the following compounds, but is not limited thereto.

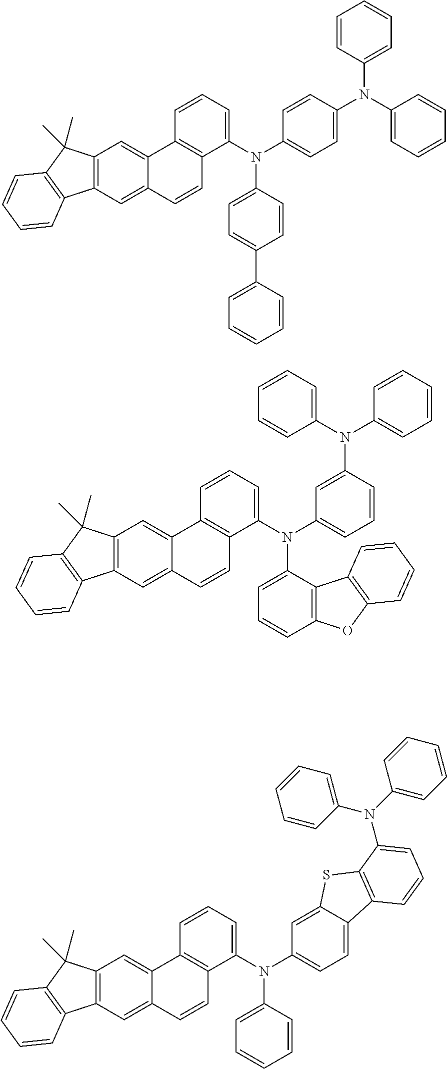

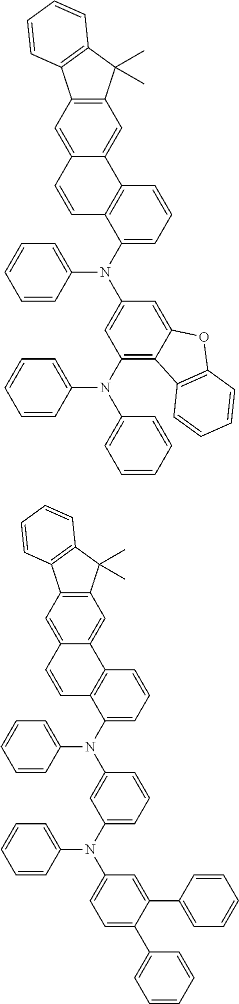 [SMILES: CC1(C)c2ccccc2-c2cc3ccc4c(N(c5ccccc5)c5cc(N(c6ccccc6)c6ccccc6)c6c(c5)oc5ccccc56)cccc4c3cc21 ; CC1(C)c2ccccc2-c2cc3ccc4c(N(c5ccccc5)c5cccc(N(c6ccccc6)c6ccc(-c7ccccc7)c(-c7ccccc7)c6)c5)cccc4c3cc21]

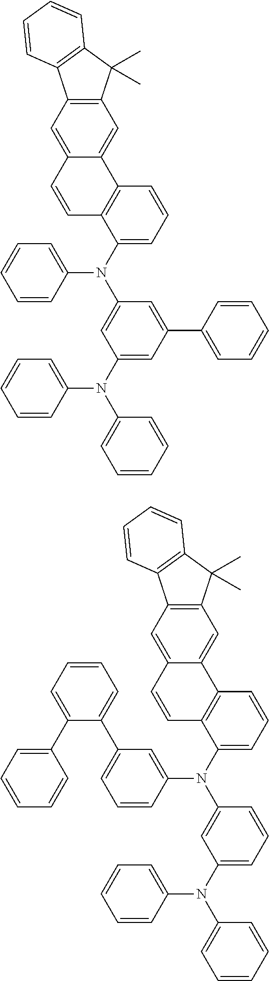

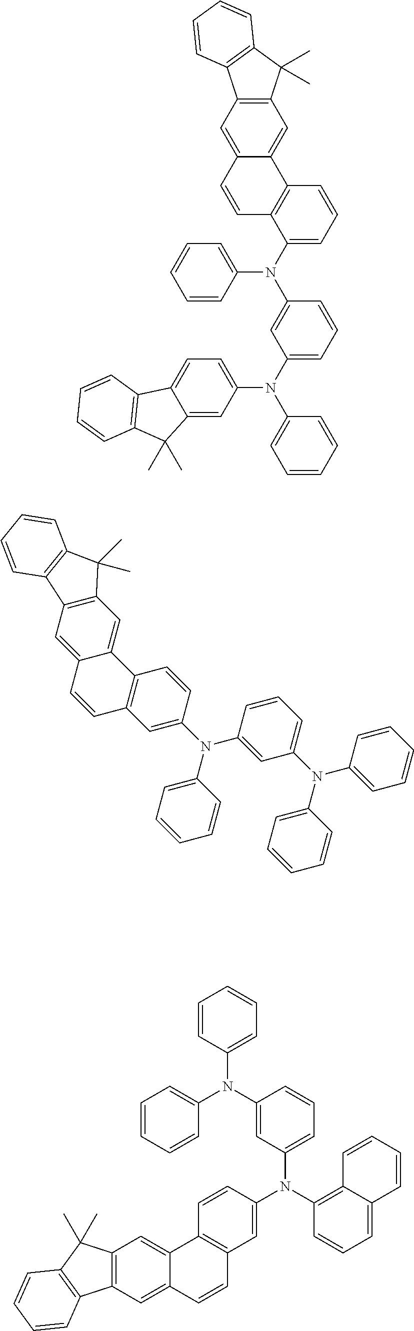 [SMILES: CC1(C)c2ccccc2-c2ccc(N(c3ccccc3)c3cccc(N(c4ccccc4)c4cccc5c4ccc4cc6c(cc45)C(C)(C)c4ccccc4-6)c3)cc21 ; CC1(C)c2ccccc2-c2cc3ccc4cc(N(c5ccccc5)c5cccc(N(c6ccccc6)c6ccccc6)c5)ccc4c3cc21 ; CC1(C)c2ccccc2-c2cc3ccc4cc(N(c5cccc(N(c6ccccc6)c6ccccc6)c5)c5cccc6ccccc56)ccc4c3cc21]

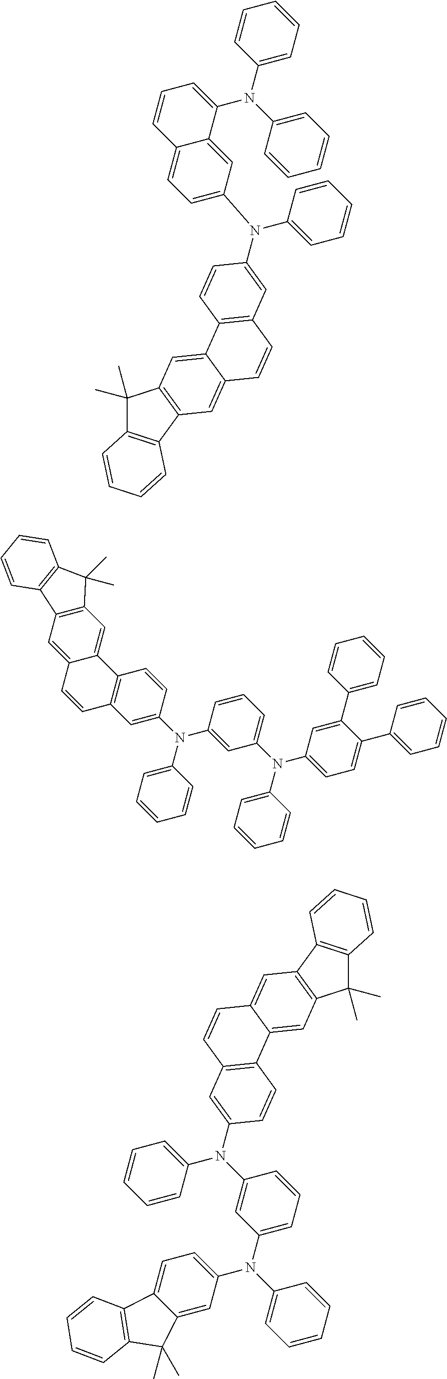 [SMILES: CC1(C)c2ccccc2-c2cc3ccc4cc(N(c5ccccc5)c5ccc6cccc(N(c7ccccc7)c7ccccc7)c6c5)ccc4c3cc21 ; CC1(C)c2ccccc2-c2cc3ccc4cc(N(c5ccccc5)c5cccc(N(c6ccccc6)c6ccc(-c7ccccc7)c(-c7ccccc7)c6)c5)ccc4c3cc21 ; CC1(C)c2ccccc2-c2ccc(N(c3ccccc3)c3cccc(N(c4ccccc4)c4ccc5c(ccc6cc7c(cc65)C(C)(C)c5ccccc5-7)c4)c3)cc21]

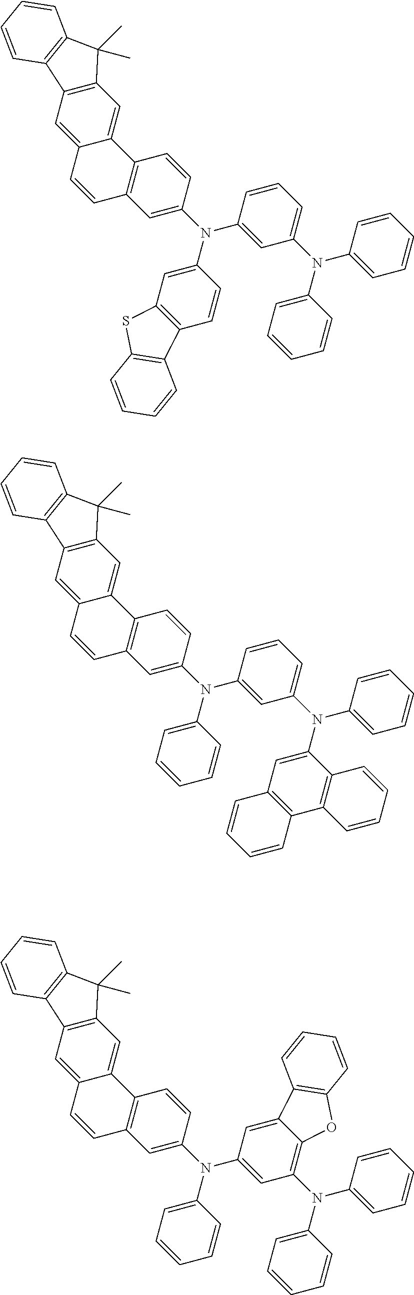 [SMILES: CC1(C)c2ccccc2-c2cc3ccc4cc(N(c5cccc(N(c6ccccc6)c6ccccc6)c5)c5ccc6c(c5)sc5ccccc56)ccc4c3cc21 ; CC1(C)c2ccccc2-c2cc3ccc4cc(N(c5ccccc5)c5cccc(N(c6ccccc6)c6cc7ccccc7c7ccccc67)c5)ccc4c3cc21 ; CC1(C)c2ccccc2-c2cc3ccc4cc(N(c5ccccc5)c5cc(N(c6ccccc6)c6ccccc6)c6oc7ccccc7c6c5)ccc4c3cc21]

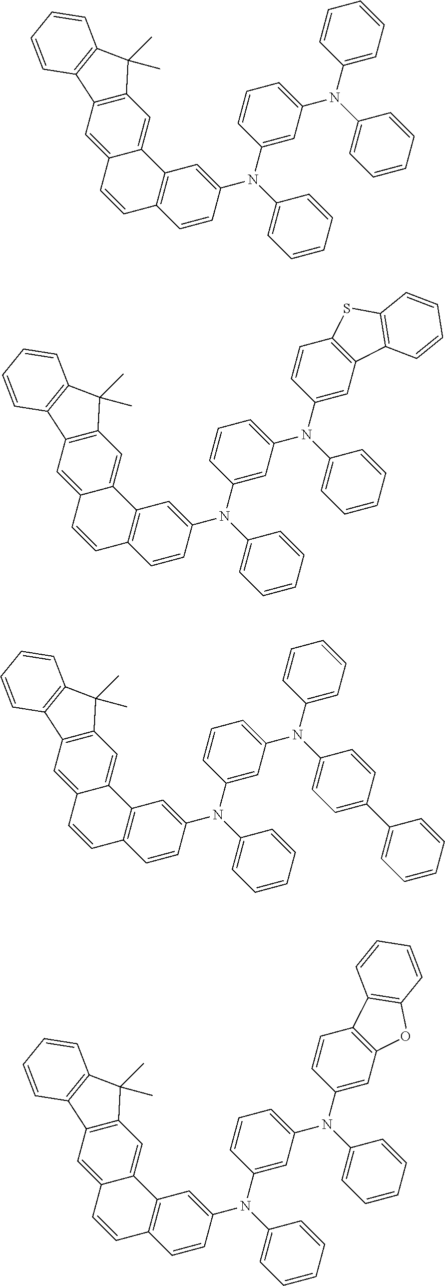 [SMILES: CC1(C)c2ccccc2-c2cc3ccc4ccc(N(c5ccccc5)c5cccc(N(c6ccccc6)c6ccccc6)c5)cc4c3cc21 ; CC1(C)c2ccccc2-c2cc3ccc4ccc(N(c5ccccc5)c5cccc(N(c6ccccc6)c6ccc7sc8ccccc8c7c6)c5)cc4c3cc21 ; CC1(C)c2ccccc2-c2cc3ccc4ccc(N(c5ccccc5)c5cccc(N(c6ccccc6)c6ccc(-c7ccccc7)cc6)c5)cc4c3cc21 ; CC1(C)c2ccccc2-c2cc3ccc4ccc(N(c5ccccc5)c5cccc(N(c6ccccc6)c6ccc7c(c6)oc6ccccc67)c5)cc4c3cc21]

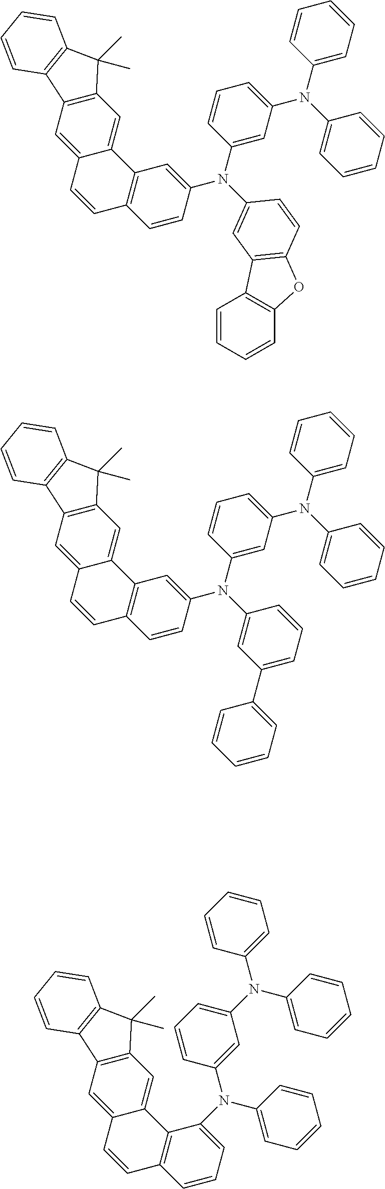 [SMILES: CC1(C)c2ccccc2-c2cc3ccc4ccc(N(c5cccc(N(c6ccccc6)c6ccccc6)c5)c5ccc6oc7ccccc7c6c5)cc4c3cc21 ; CC1(C)c2ccccc2-c2cc3ccc4ccc(N(c5cccc(-c6ccccc6)c5)c5cccc(N(c6ccccc6)c6ccccc6)c5)cc4c3cc21 ; CC1(C)c2ccccc2-c2cc3ccc4cccc(N(c5ccccc5)c5cccc(N(c6ccccc6)c6ccccc6)c5)c4c3cc21]

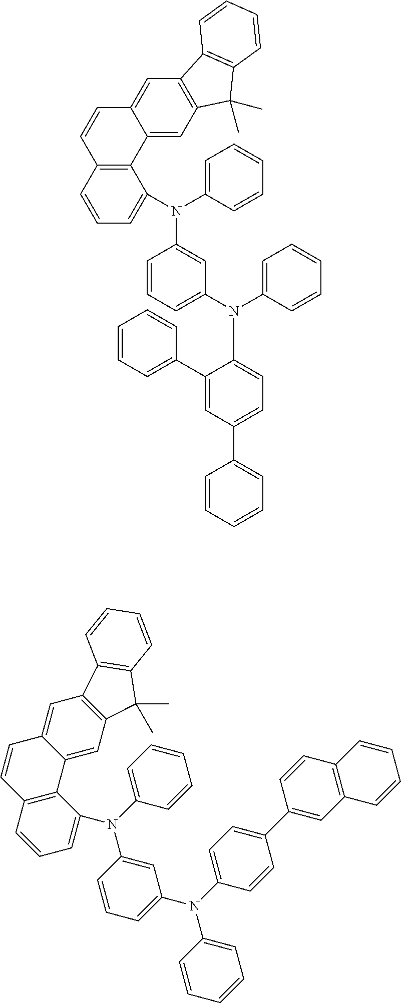 [SMILES: CC1(C)c2ccccc2-c2cc3ccc4cccc(N(c5ccccc5)c5cccc(N(c6ccccc6)c6ccc(-c7ccccc7)cc6-c6ccccc6)c5)c4c3cc21 ; CC1(C)c2ccccc2-c2cc3ccc4cccc(N(c5ccccc5)c5cccc(N(c6ccccc6)c6ccc(-c7ccc8ccccc8c7)cc6)c5)c4c3cc21]

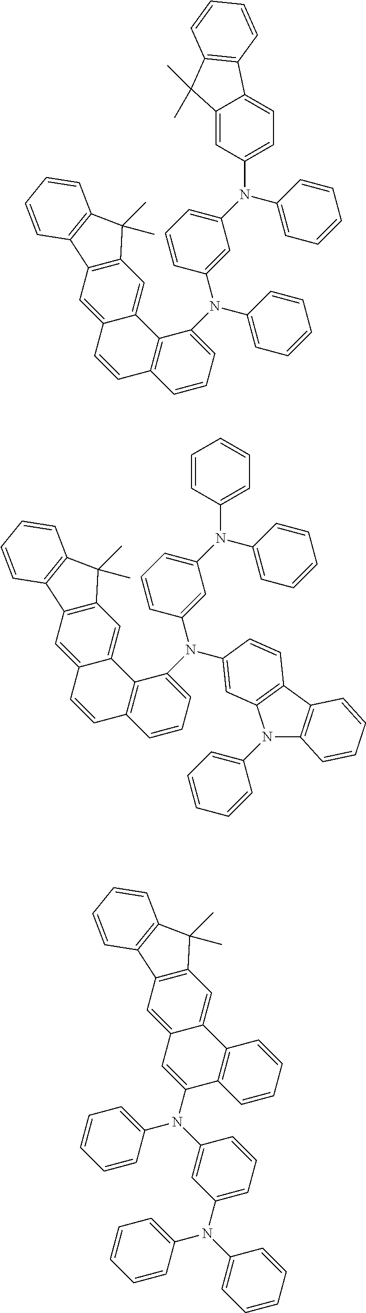 [SMILES: CC1(C)c2ccccc2-c2ccc(N(c3ccccc3)c3cccc(N(c4ccccc4)c4cccc5ccc6cc7c(cc6c45)C(C)(C)c4ccccc4-7)c3)cc21 ; CC1(C)c2ccccc2-c2cc3ccc4cccc(N(c5cccc(N(c6ccccc6)c6ccccc6)c5)c5ccc6c7ccccc7n(-c7ccccc7)c6c5)c4c3cc21 ; CC1(C)c2ccccc2-c2cc3cc(N(c4ccccc4)c4cccc(N(c5ccccc5)c5ccccc5)c4)c4ccccc4c3cc21]

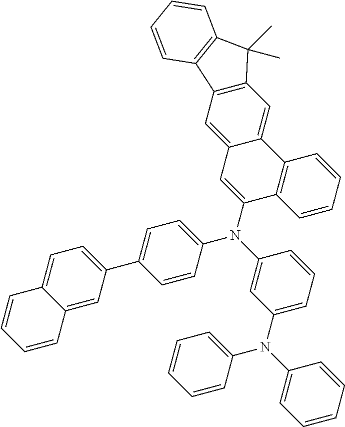 [SMILES: CC1(C)c2ccccc2-c2cc3cc(N(c4ccc(-c5ccc6ccccc6c5)cc4)c4cccc(N(c5ccccc5)c5ccccc5)c4)c4ccccc4c3cc21]

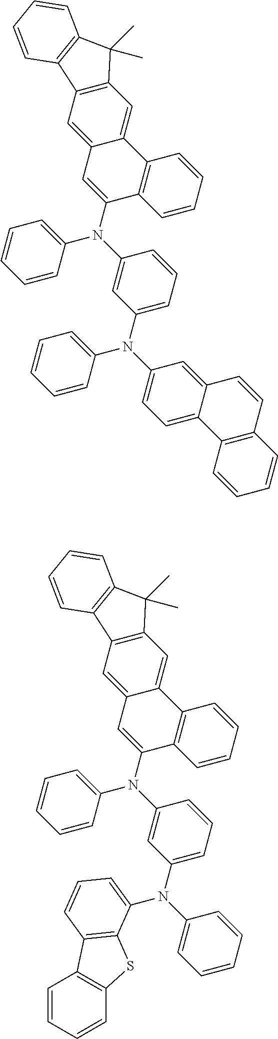 [SMILES: CC1(C)c2ccccc2-c2cc3cc(N(c4ccccc4)c4cccc(N(c5ccccc5)c5ccc6c(ccc7ccccc76)c5)c4)c4ccccc4c3cc21 ; CC1(C)c2ccccc2-c2cc3cc(N(c4ccccc4)c4cccc(N(c5ccccc5)c5cccc6c5sc5ccccc56)c4)c4ccccc4c3cc21]

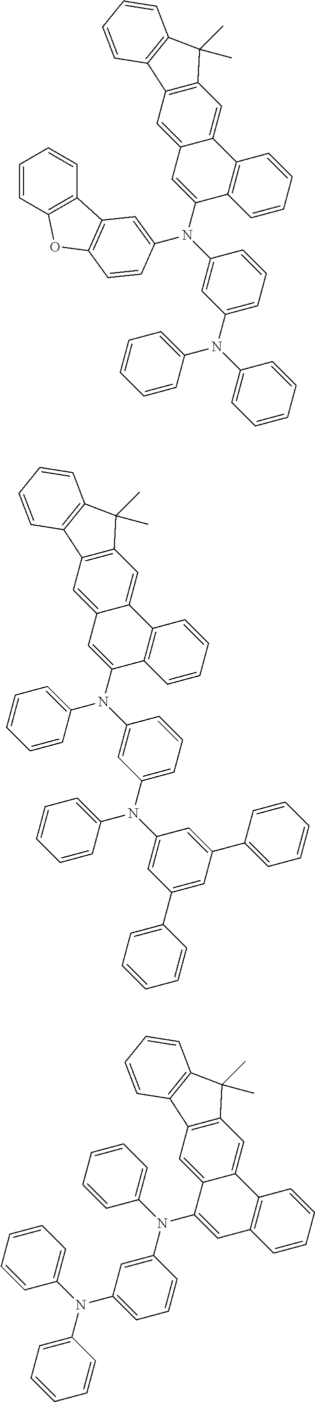 [SMILES: CC1(C)c2ccccc2-c2cc3cc(N(c4cccc(N(c5ccccc5)c5ccccc5)c4)c4ccc5oc6ccccc6c5c4)c4ccccc4c3cc21 ; CC1(C)c2ccccc2-c2cc3cc(N(c4ccccc4)c4cccc(N(c5ccccc5)c5cc(-c6ccccc6)cc(-c6ccccc6)c5)c4)c4ccccc4c3cc21 ; CC1(C)c2ccccc2-c2cc3c(N(c4ccccc4)c4cccc(N(c5ccccc5)c5ccccc5)c4)cc4ccccc4c3cc21]

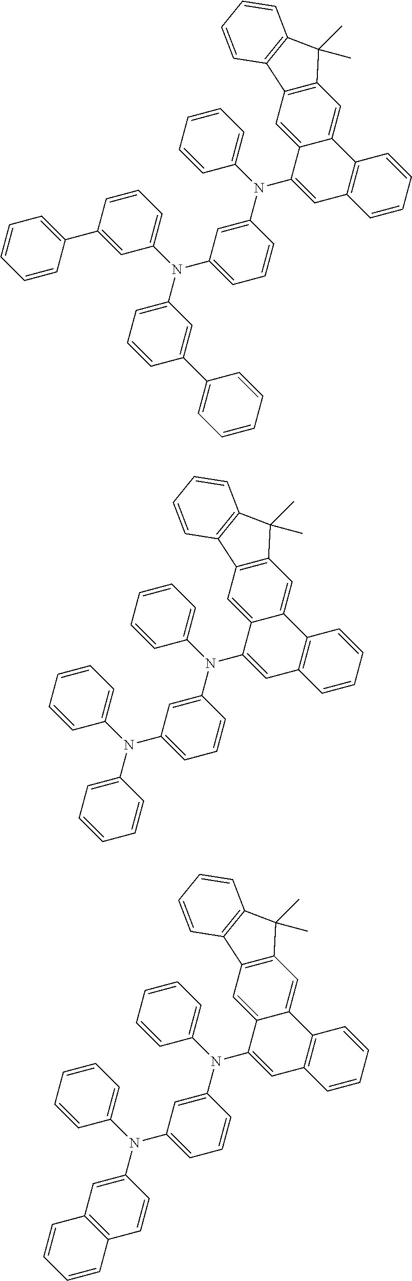

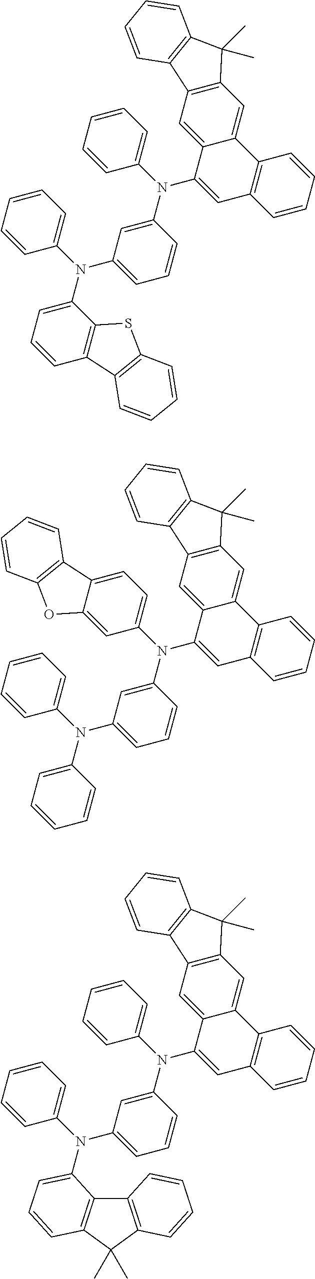

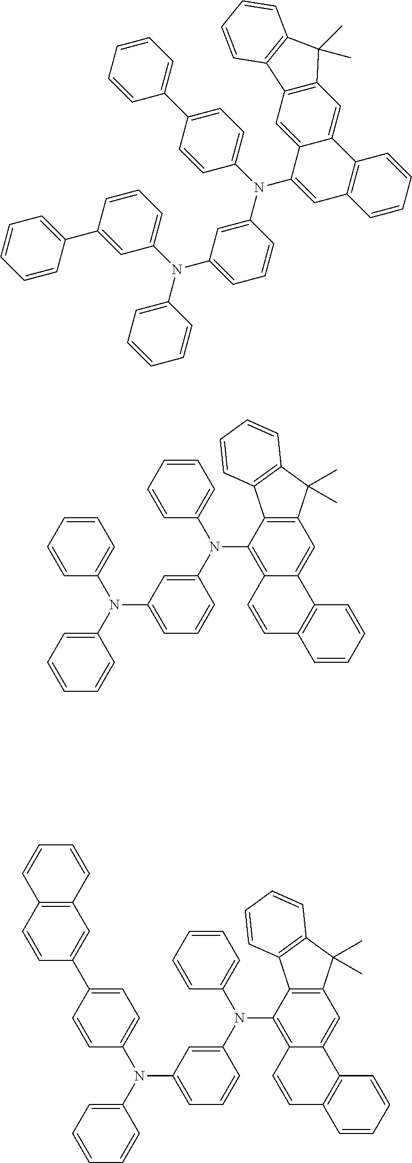 [SMILES: CC1(C)c2ccccc2-c2cc3c(N(c4ccc(-c5ccccc5)cc4)c4cccc(N(c5ccccc5)c5cccc(-c6ccccc6)c5)c4)cc4ccccc4c3cc21 ; CC1(C)c2ccccc2-c2c1cc1c(ccc3ccccc31)c2N(c1ccccc1)c1cccc(N(c2ccccc2)c2ccccc2)c1 ; CC1(C)c2ccccc2-c2c1cc1c(ccc3ccccc31)c2N(c1ccccc1)c1cccc(N(c2ccccc2)c2ccc(-c3ccc4ccccc4c3)cc2)c1]

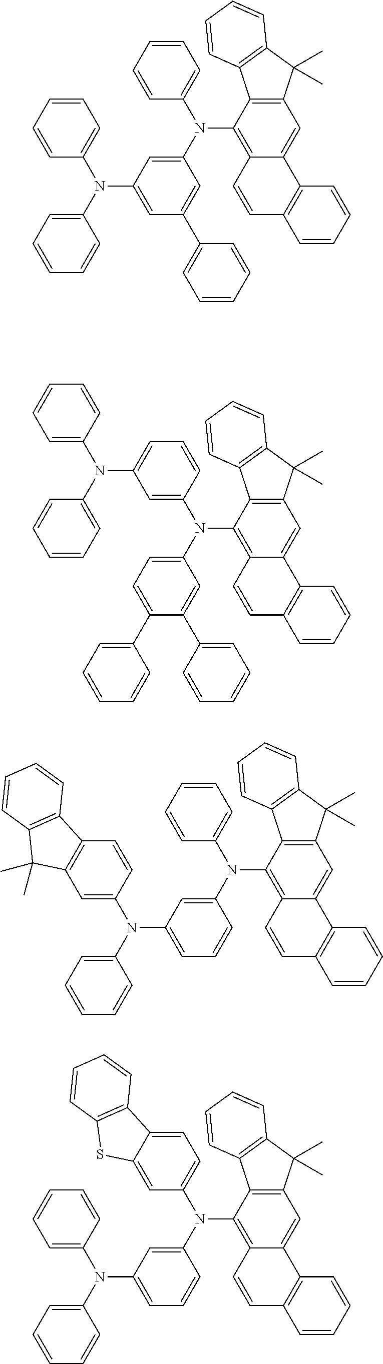

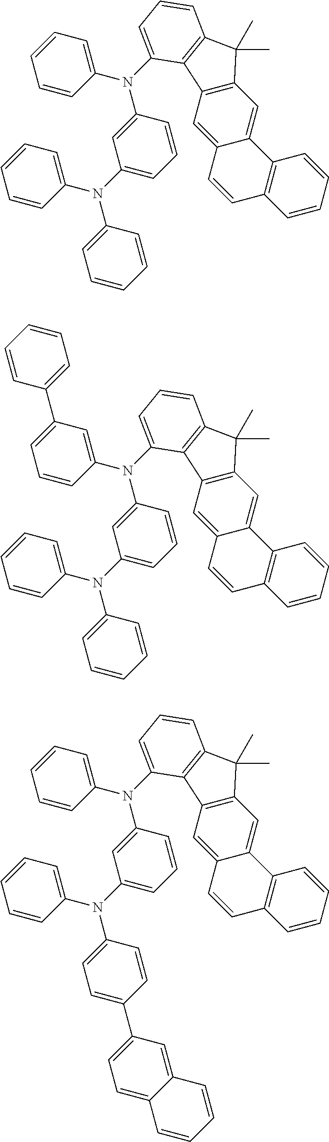 [SMILES: CC1(C)c2cc3c(ccc4ccccc43)cc2-c2c(N(c3ccccc3)c3cccc(N(c4ccccc4)c4ccccc4)c3)cccc21 ; CC1(C)c2cc3c(ccc4ccccc43)cc2-c2c(N(c3cccc(-c4ccccc4)c3)c3cccc(N(c4ccccc4)c4ccccc4)c3)cccc21 ; CC1(C)c2cc3c(ccc4ccccc43)cc2-c2c(N(c3ccccc3)c3cccc(N(c4ccccc4)c4ccc(-c5ccc6ccccc6c5)cc4)c3)cccc21]

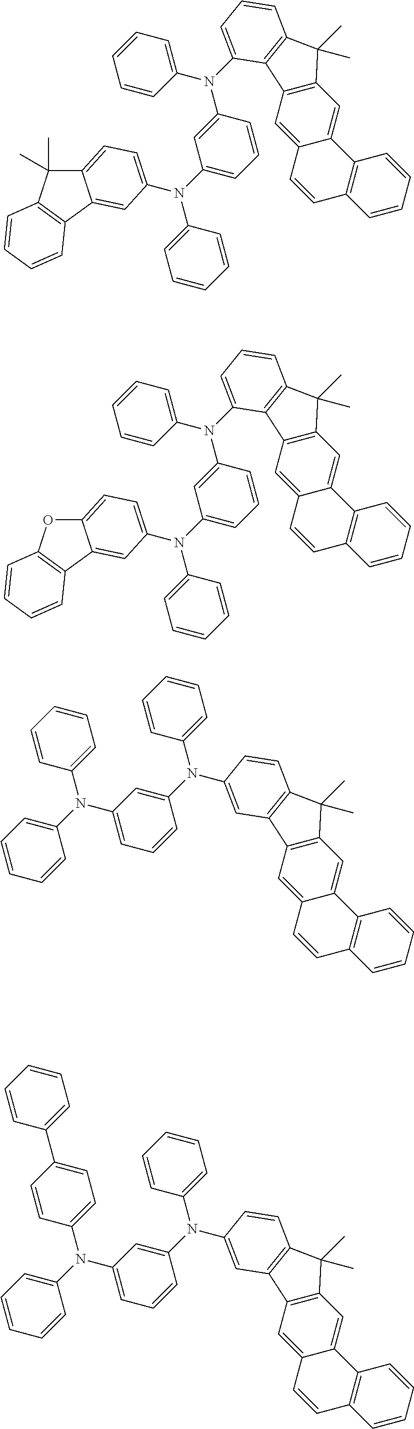 [SMILES: CC1(C)c2ccccc2-c2cc(N(c3ccccc3)c3cccc(N(c4ccccc4)c4cccc5c4-c4cc6ccc7ccccc7c6cc4C5(C)C)c3)ccc21 ; CC1(C)c2cc3c(ccc4ccccc43)cc2-c2c(N(c3ccccc3)c3cccc(N(c4ccccc4)c4ccc5oc6ccccc6c5c4)c3)cccc21 ; CC1(C)c2ccc(N(c3ccccc3)c3cccc(N(c4ccccc4)c4ccccc4)c3)cc2-c2cc3ccc4ccccc4c3cc21 ; CC1(C)c2ccc(N(c3ccccc3)c3cccc(N(c4ccccc4)c4ccc(-c5ccccc5)cc4)c3)cc2-c2cc3ccc4ccccc4c3cc21]

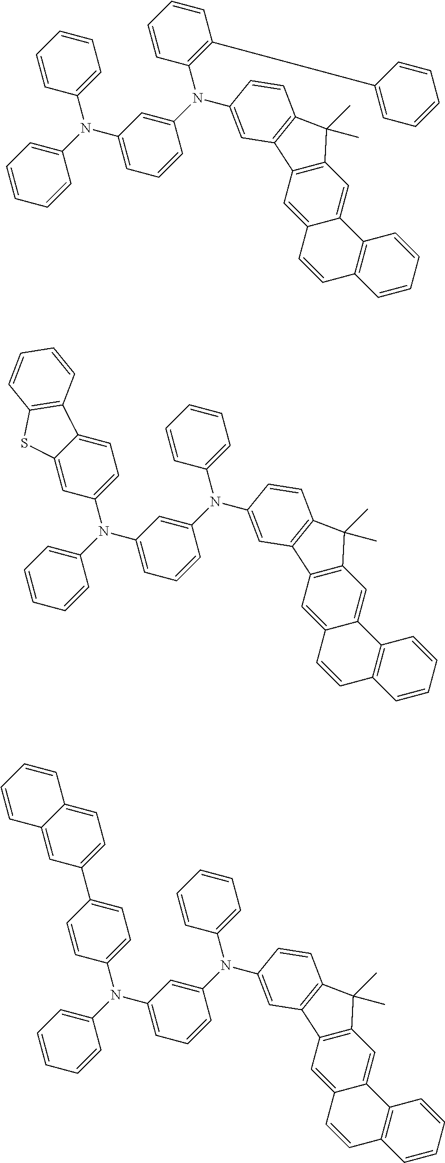 [SMILES: CC1(C)c2ccc(N(c3cccc(N(c4ccccc4)c4ccccc4)c3)c3ccccc3-c3ccccc3)cc2-c2cc3ccc4ccccc4c3cc21 ; CC1(C)c2ccc(N(c3ccccc3)c3cccc(N(c4ccccc4)c4ccc5c(c4)sc4ccccc45)c3)cc2-c2cc3ccc4ccccc4c3cc21 ; CC1(C)c2ccc(N(c3ccccc3)c3cccc(N(c4ccccc4)c4ccc(-c5ccc6ccccc6c5)cc4)c3)cc2-c2cc3ccc4ccccc4c3cc21]

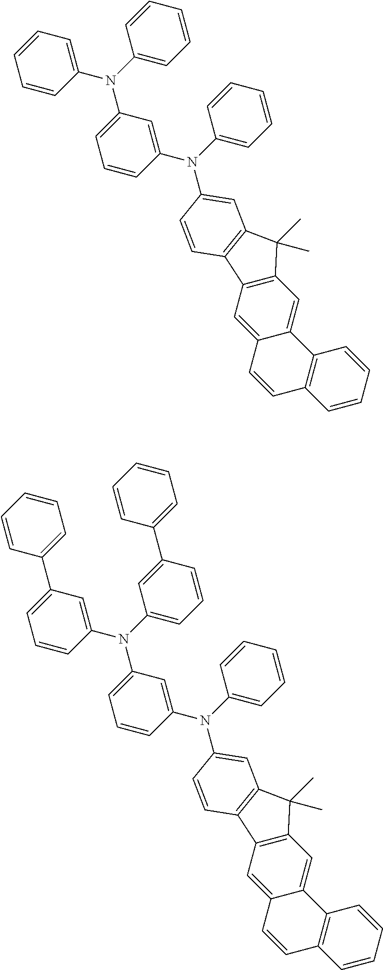 [SMILES: CC1(C)c2cc(N(c3ccccc3)c3cccc(N(c4ccccc4)c4ccccc4)c3)ccc2-c2cc3ccc4ccccc4c3cc21 ; CC1(C)c2cc(N(c3ccccc3)c3cccc(N(c4cccc(-c5ccccc5)c4)c4cccc(-c5ccccc5)c4)c3)ccc2-c2cc3ccc4ccccc4c3cc21]

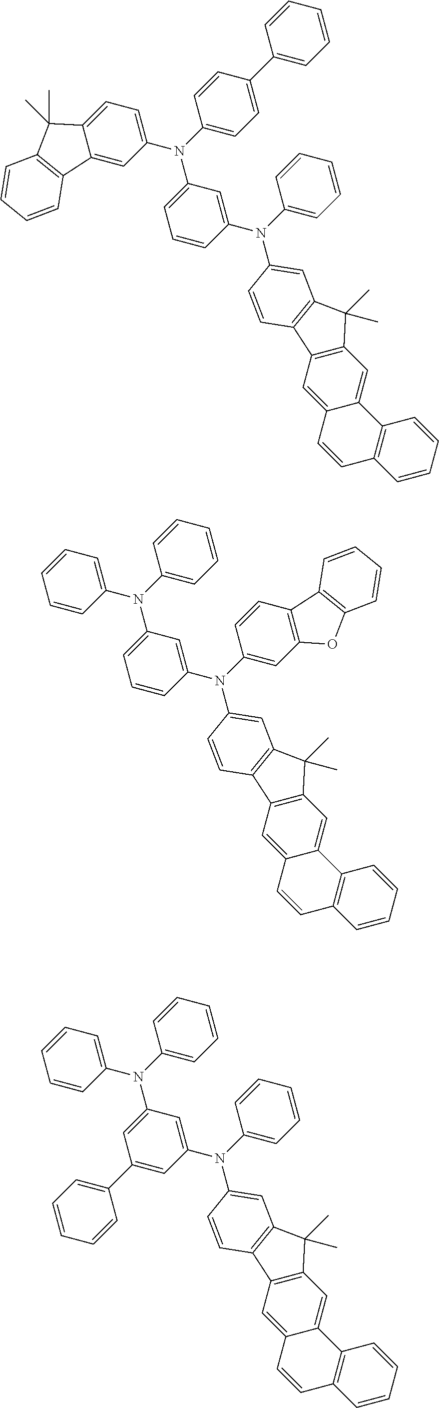 [SMILES: CC1(C)c2ccccc2-c2cc(N(c3ccc(-c4ccccc4)cc3)c3cccc(N(c4ccccc4)c4ccc5c(c4)C(C)(C)c4cc6c(ccc7ccccc76)cc4-5)c3)ccc21 ; CC1(C)c2cc(N(c3cccc(N(c4ccccc4)c4ccccc4)c3)c3ccc4c(c3)oc3ccccc34)ccc2-c2cc3ccc4ccccc4c3cc21 ; CC1(C)c2cc(N(c3ccccc3)c3cc(-c4ccccc4)cc(N(c4ccccc4)c4ccccc4)c3)ccc2-c2cc3ccc4ccccc4c3cc21]

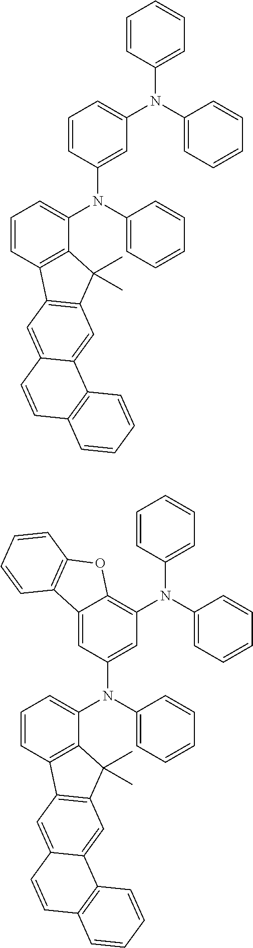 [SMILES: CC1(C)c2cc3c(ccc4ccccc43)cc2-c2cccc(N(c3ccccc3)c3cccc(N(c4ccccc4)c4ccccc4)c3)c21 ; CC1(C)c2cc3c(ccc4ccccc43)cc2-c2cccc(N(c3ccccc3)c3cc(N(c4ccccc4)c4ccccc4)c4oc5ccccc5c4c3)c21]

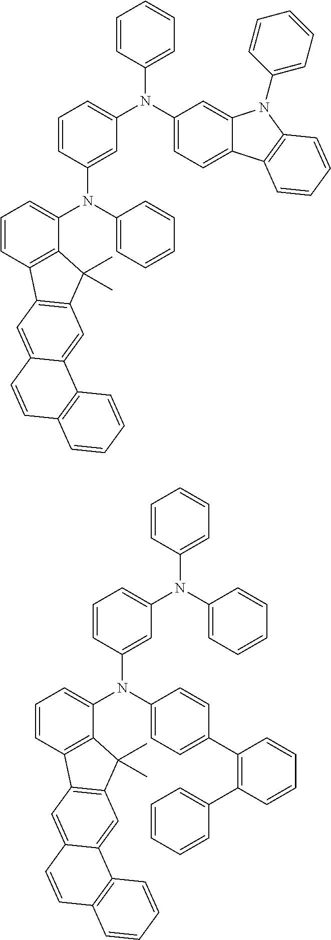 [SMILES: CC1(C)c2cc3c(ccc4ccccc43)cc2-c2cccc(N(c3ccccc3)c3cccc(N(c4ccccc4)c4ccc5c6ccccc6n(-c6ccccc6)c5c4)c3)c21 ; CC1(C)c2cc3c(ccc4ccccc43)cc2-c2cccc(N(c3ccc(-c4ccccc4-c4ccccc4)cc3)c3cccc(N(c4ccccc4)c4ccccc4)c3)c21]

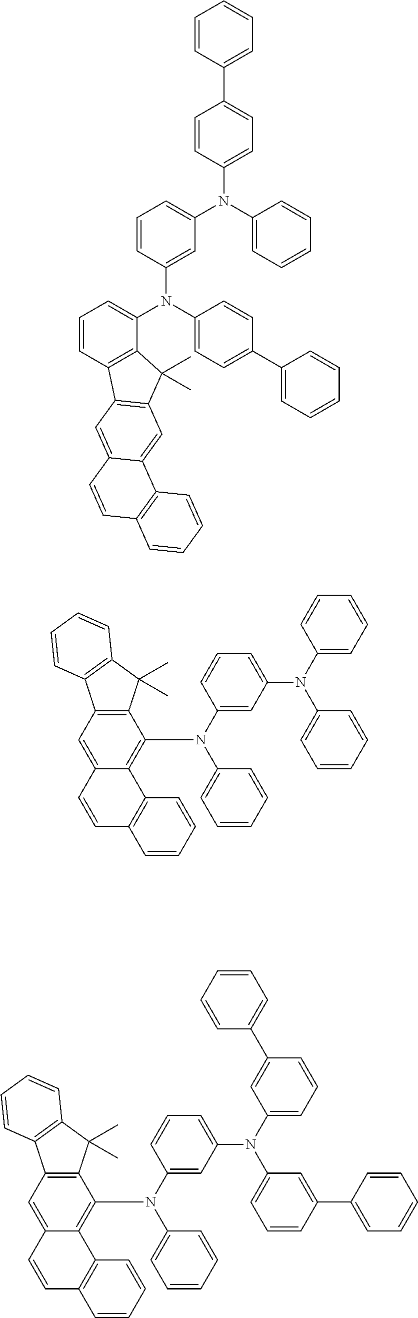 [SMILES: CC1(C)c2cc3c(ccc4ccccc43)cc2-c2cccc(N(c3ccc(-c4ccccc4)cc3)c3cccc(N(c4ccccc4)c4ccc(-c5ccccc5)cc4)c3)c21 ; CC1(C)c2ccccc2-c2cc3ccc4ccccc4c3c(N(c3ccccc3)c3cccc(N(c4ccccc4)c4ccccc4)c3)c21 ; CC1(C)c2ccccc2-c2cc3ccc4ccccc4c3c(N(c3ccccc3)c3cccc(N(c4cccc(-c5ccccc5)c4)c4cccc(-c5ccccc5)c4)c3)c21]

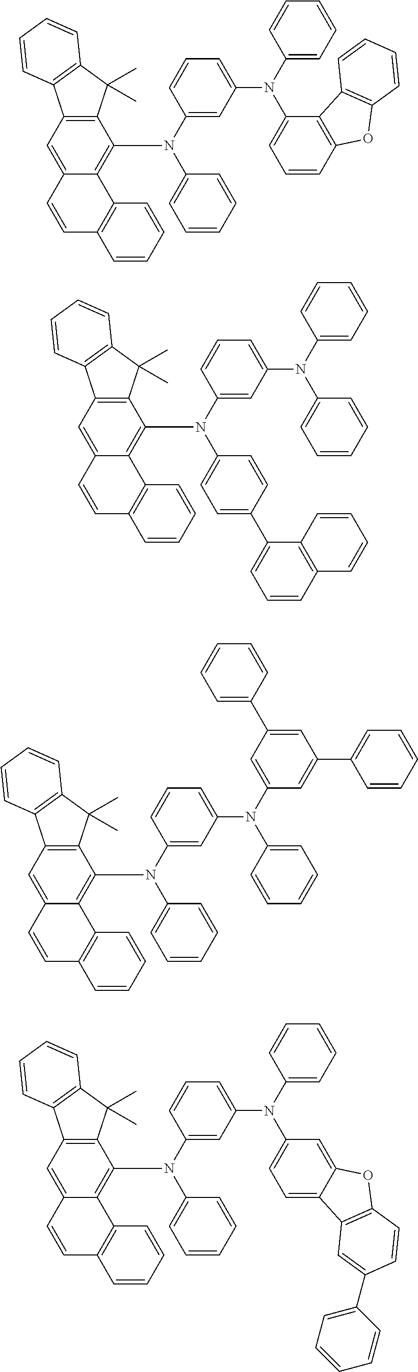

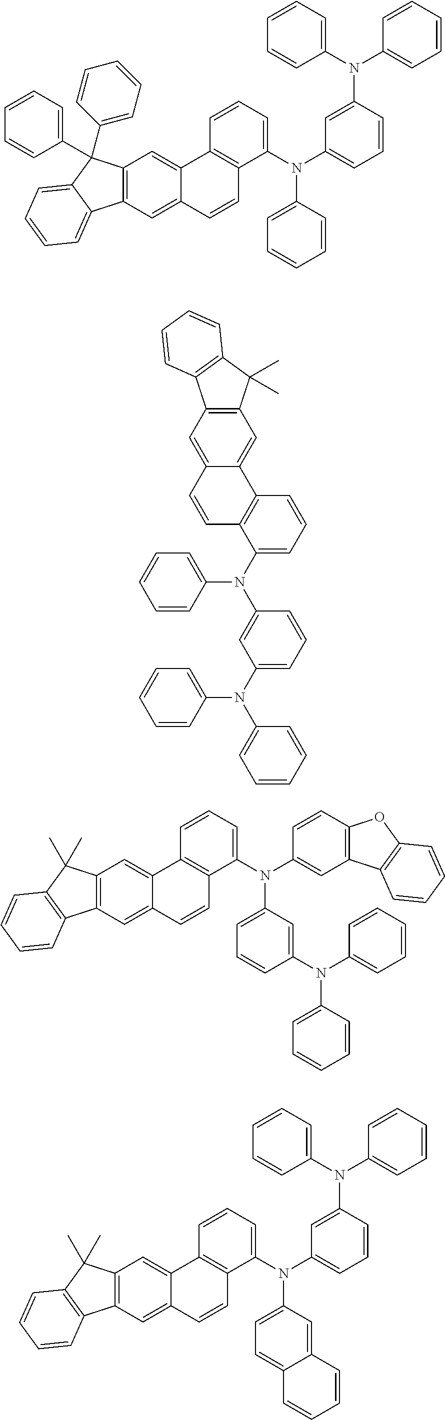

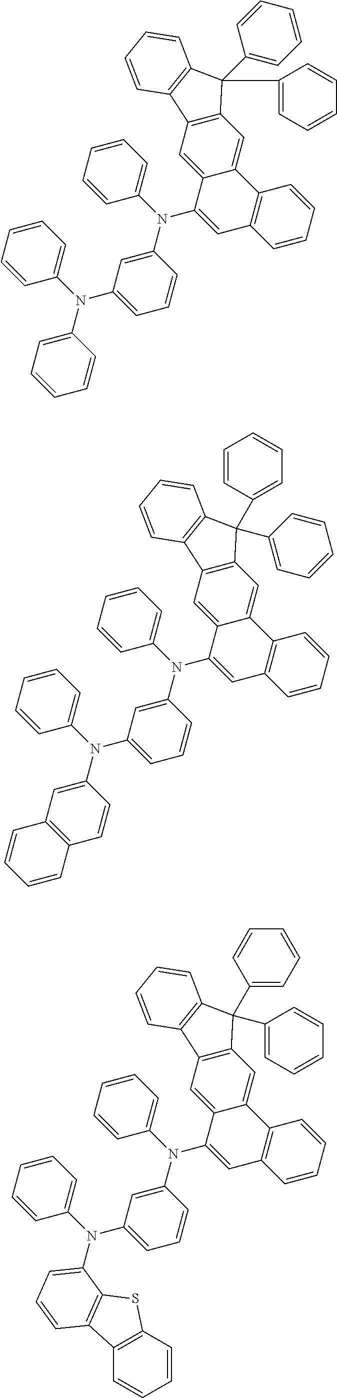 [SMILES: c1ccc(N(c2ccccc2)c2cccc(N(c3ccccc3)c3cc4ccccc4c4cc5c(cc34)-c3ccccc3C5(c3ccccc3)c3ccccc3)c2)cc1 ; c1ccc(N(c2cccc(N(c3ccccc3)c3cc4ccccc4c4cc5c(cc34)-c3ccccc3C5(c3ccccc3)c3ccccc3)c2)c2ccc3ccccc3c2)cc1 ; c1ccc(N(c2cccc(N(c3ccccc3)c3cccc4c3sc3ccccc34)c2)c2cc3ccccc3c3cc4c(cc23)-c2ccccc2C4(c2ccccc2)c2ccccc2)cc1]

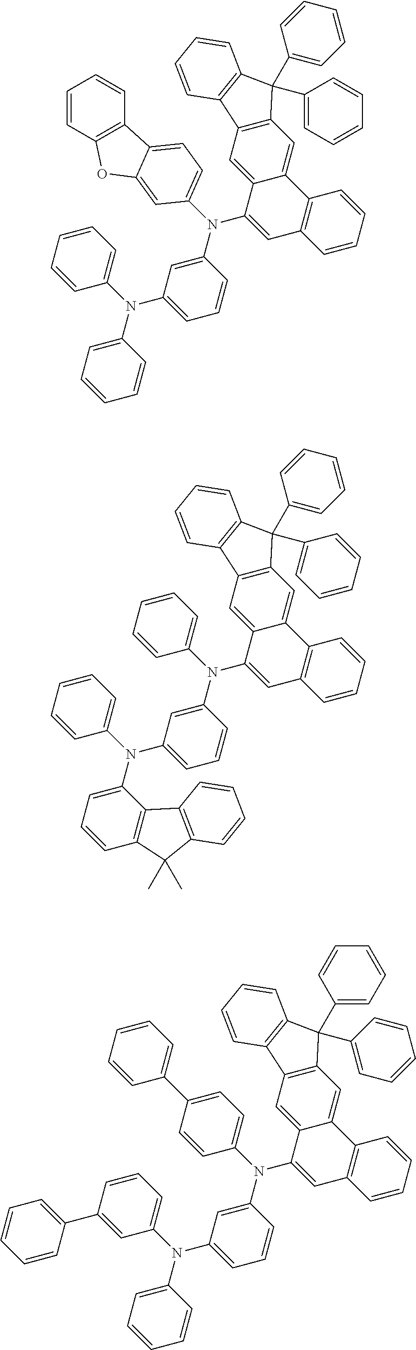 [SMILES: c1ccc(N(c2ccccc2)c2cccc(N(c3ccc4c(c3)oc3ccccc34)c3cc4ccccc4c4cc5c(cc34)-c3ccccc3C5(c3ccccc3)c3ccccc3)c2)cc1 ; CC1(C)c2ccccc2-c2c(N(c3ccccc3)c3cccc(N(c4ccccc4)c4cc5ccccc5c5cc6c(cc45)-c4ccccc4C6(c4ccccc4)c4ccccc4)c3)cccc21 ; c1ccc(-c2ccc(N(c3cccc(N(c4ccccc4)c4cccc(-c5ccccc5)c4)c3)c3cc4ccccc4c4cc5c(cc34)-c3ccccc3C5(c3ccccc3)c3ccccc3)cc2)cc1]

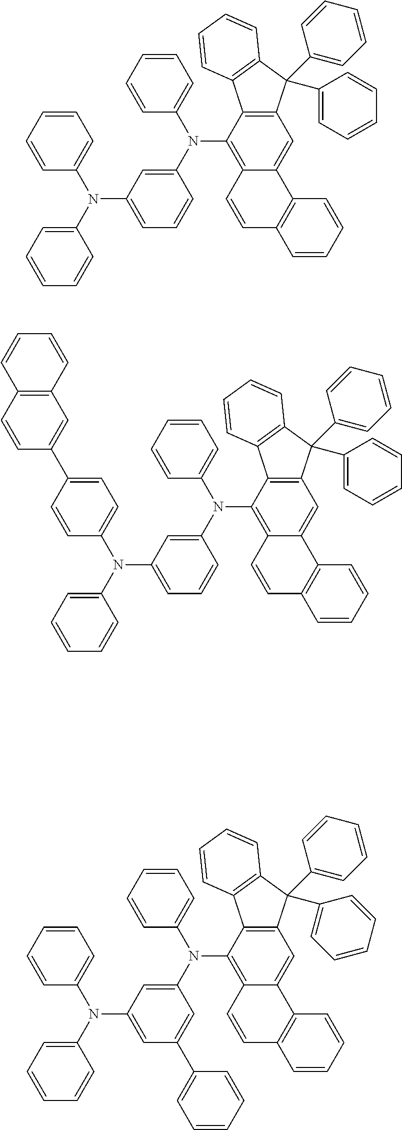 [SMILES: c1ccc(N(c2ccccc2)c2cccc(N(c3ccccc3)c3c4c(cc5c3ccc3ccccc35)C(c3ccccc3)(c3ccccc3)c3ccccc3-4)c2)cc1 ; c1ccc(N(c2ccc(-c3ccc4ccccc4c3)cc2)c2cccc(N(c3ccccc3)c3c4c(cc5c3ccc3ccccc35)C(c3ccccc3)(c3ccccc3)c3ccccc3-4)c2)cc1 ; c1ccc(-c2cc(N(c3ccccc3)c3ccccc3)cc(N(c3ccccc3)c3c4c(cc5c3ccc3ccccc35)C(c3ccccc3)(c3ccccc3)c3ccccc3-4)c2)cc1]

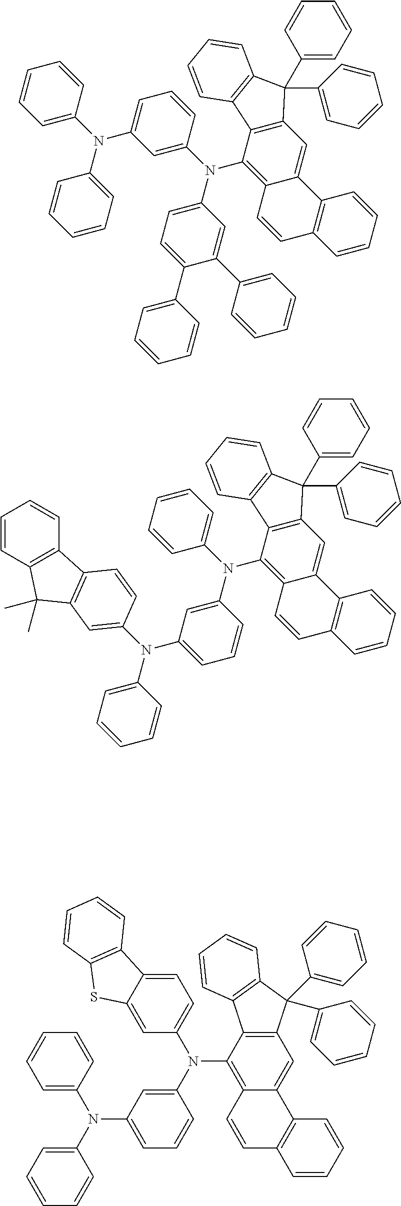 [SMILES: c1ccc(-c2ccc(N(c3cccc(N(c4ccccc4)c4ccccc4)c3)c3c4c(cc5c3ccc3ccccc35)C(c3ccccc3)(c3ccccc3)c3ccccc3-4)cc2-c2ccccc2)cc1 ; CC1(C)c2ccccc2-c2ccc(N(c3ccccc3)c3cccc(N(c4ccccc4)c4c5c(cc6c4ccc4ccccc46)C(c4ccccc4)(c4ccccc4)c4ccccc4-5)c3)cc21 ; c1ccc(N(c2ccccc2)c2cccc(N(c3ccc4c(c3)sc3ccccc34)c3c4c(cc5c3ccc3ccccc35)C(c3ccccc3)(c3ccccc3)c3ccccc3-4)c2)cc1]

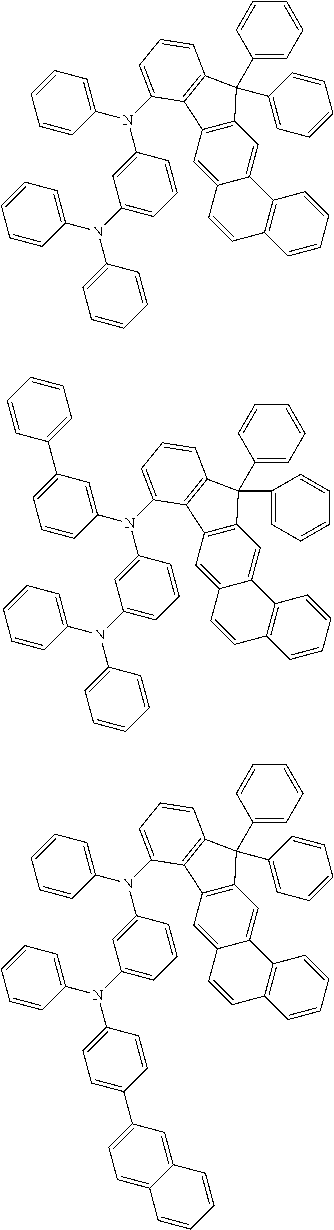 [SMILES: c1ccc(N(c2ccccc2)c2cccc(N(c3ccccc3)c3cccc4c3-c3cc5ccc6ccccc6c5cc3C4(c3ccccc3)c3ccccc3)c2)cc1 ; c1ccc(-c2cccc(N(c3cccc(N(c4ccccc4)c4ccccc4)c3)c3cccc4c3-c3cc5ccc6ccccc6c5cc3C4(c3ccccc3)c3ccccc3)c2)cc1 ; c1ccc(N(c2ccc(-c3ccc4ccccc4c3)cc2)c2cccc(N(c3ccccc3)c3cccc4c3-c3cc5ccc6ccccc6c5cc3C4(c3ccccc3)c3ccccc3)c2)cc1]

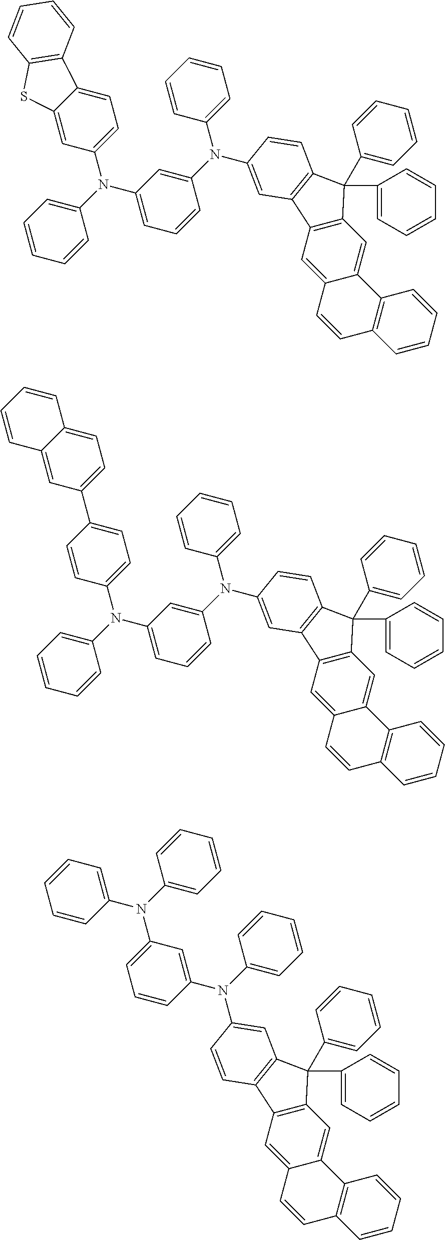 [SMILES: c1ccc(N(c2cccc(N(c3ccccc3)c3ccc4c(c3)sc3ccccc34)c2)c2ccc3c(c2)-c2cc4ccc5ccccc5c4cc2C3(c2ccccc2)c2ccccc2)cc1 ; c1ccc(N(c2ccc(-c3ccc4ccccc4c3)cc2)c2cccc(N(c3ccccc3)c3ccc4c(c3)-c3cc5ccc6ccccc6c5cc3C4(c3ccccc3)c3ccccc3)c2)cc1 ; c1ccc(N(c2ccccc2)c2cccc(N(c3ccccc3)c3ccc4c(c3)C(c3ccccc3)(c3ccccc3)c3cc5c(ccc6ccccc65)cc3-4)c2)cc1]

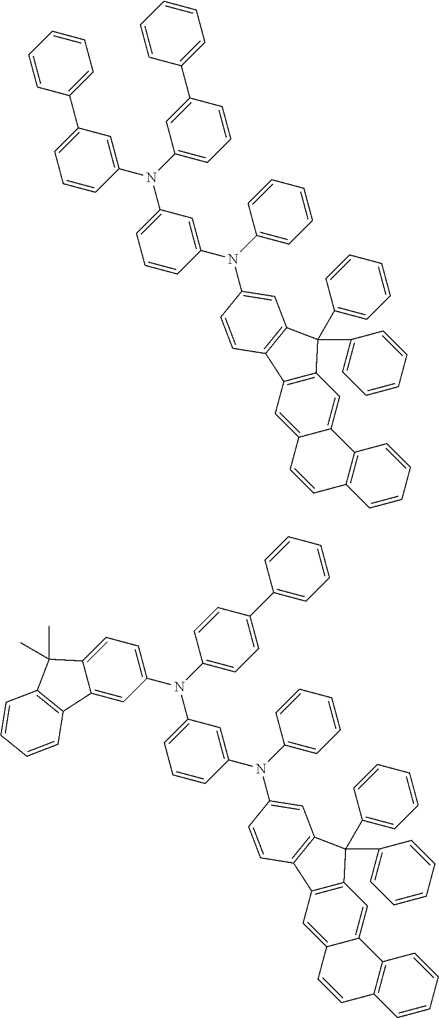 [SMILES: c1ccc(-c2cccc(N(c3cccc(-c4ccccc4)c3)c3cccc(N(c4ccccc4)c4ccc5c(c4)C(c4ccccc4)(c4ccccc4)c4cc6c(ccc7ccccc76)cc4-5)c3)c2)cc1 ; CC1(C)c2ccccc2-c2cc(N(c3ccc(-c4ccccc4)cc3)c3cccc(N(c4ccccc4)c4ccc5c(c4)C(c4ccccc4)(c4ccccc4)c4cc6c(ccc7ccccc76)cc4-5)c3)ccc21]

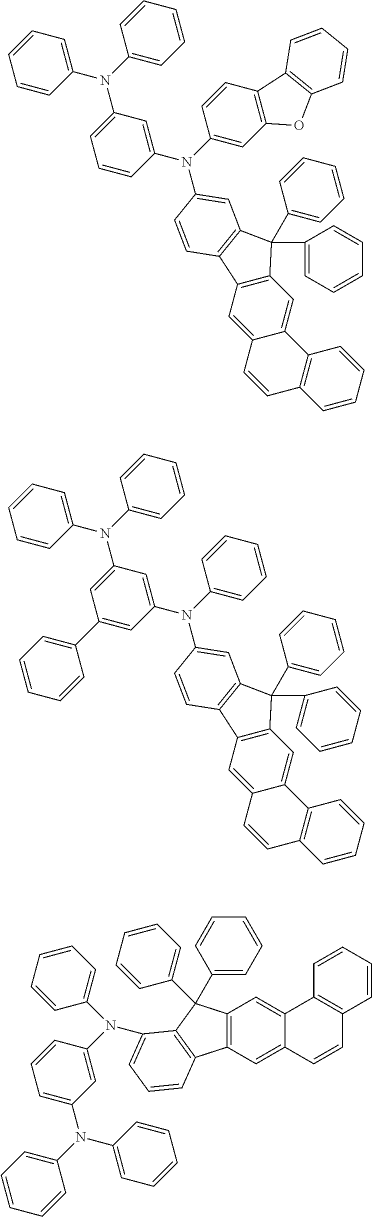 [SMILES: c1ccc(N(c2ccccc2)c2cccc(N(c3ccc4c(c3)C(c3ccccc3)(c3ccccc3)c3cc5c(ccc6ccccc65)cc3-4)c3ccc4c(c3)oc3ccccc34)c2)cc1 ; c1ccc(-c2cc(N(c3ccccc3)c3ccccc3)cc(N(c3ccccc3)c3ccc4c(c3)C(c3ccccc3)(c3ccccc3)c3cc5c(ccc6ccccc65)cc3-4)c2)cc1 ; c1ccc(N(c2ccccc2)c2cccc(N(c3ccccc3)c3cccc4c3C(c3ccccc3)(c3ccccc3)c3cc5c(ccc6ccccc65)cc3-4)c2)cc1]

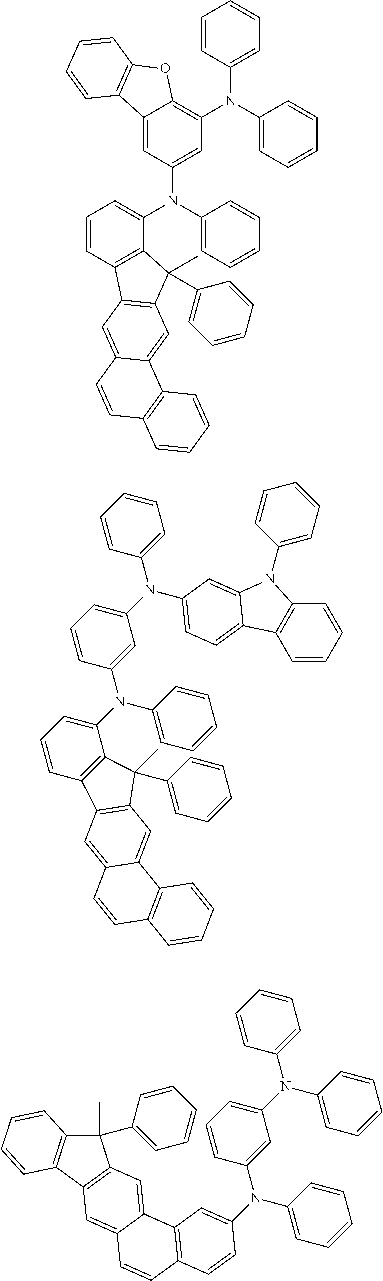 [SMILES: CC1(c2ccccc2)c2cc3c(ccc4ccccc43)cc2-c2cccc(N(c3ccccc3)c3cc(N(c4ccccc4)c4ccccc4)c4oc5ccccc5c4c3)c21 ; CC1(c2ccccc2)c2cc3c(ccc4ccccc43)cc2-c2cccc(N(c3ccccc3)c3cccc(N(c4ccccc4)c4ccc5c6ccccc6n(-c6ccccc6)c5c4)c3)c21 ; CC1(c2ccccc2)c2ccccc2-c2cc3ccc4ccc(N(c5ccccc5)c5cccc(N(c6ccccc6)c6ccccc6)c5)cc4c3cc21]

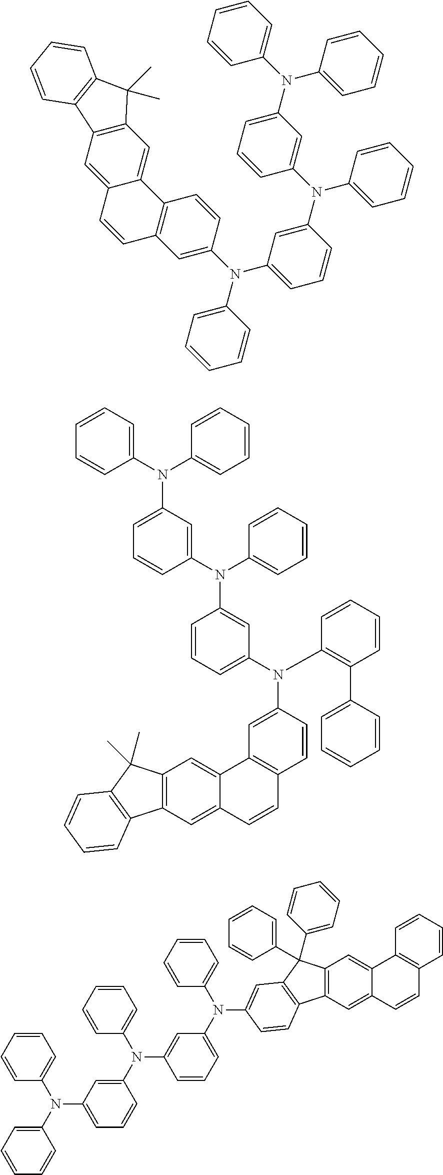

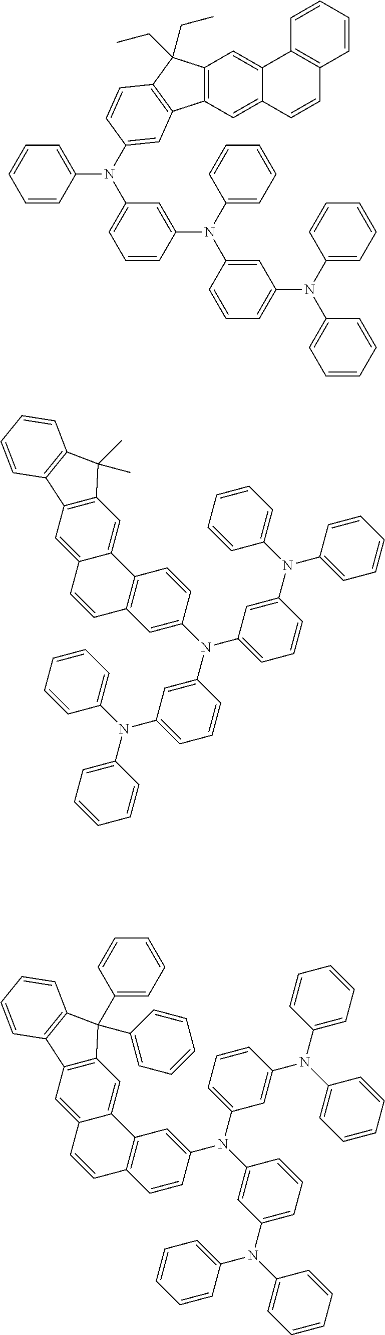 [SMILES: CCC1(CC)c2ccc(N(c3ccccc3)c3cccc(N(c4ccccc4)c4cccc(N(c5ccccc5)c5ccccc5)c4)c3)cc2-c2cc3ccc4ccccc4c3cc21 ; CC1(C)c2ccccc2-c2cc3ccc4cc(N(c5cccc(N(c6ccccc6)c6ccccc6)c5)c5cccc(N(c6ccccc6)c6ccccc6)c5)ccc4c3cc21 ; c1ccc(N(c2ccccc2)c2cccc(N(c3cccc(N(c4ccccc4)c4ccccc4)c3)c3ccc4ccc5cc6c(cc5c4c3)C(c3ccccc3)(c3ccccc3)c3ccccc3-6)c2)cc1]

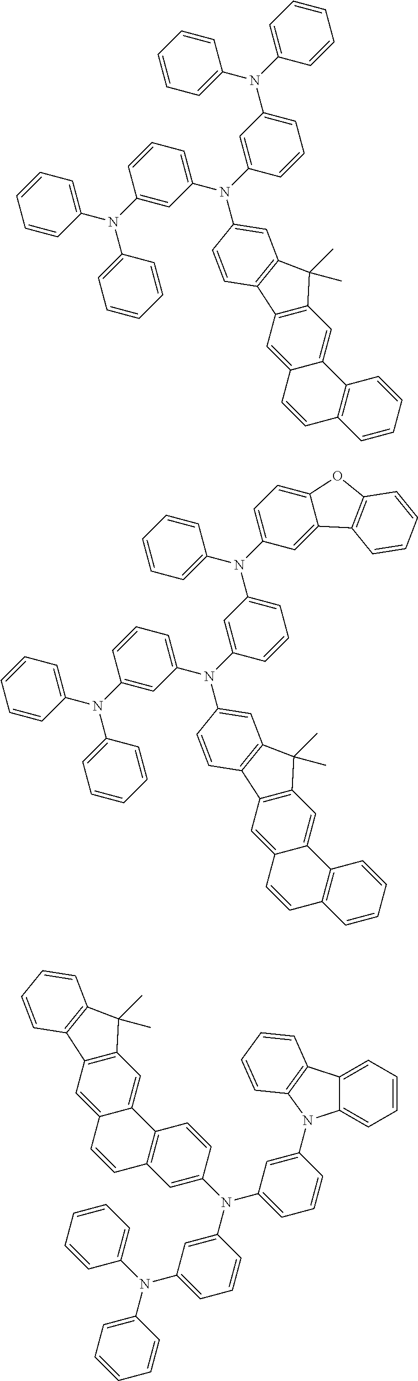

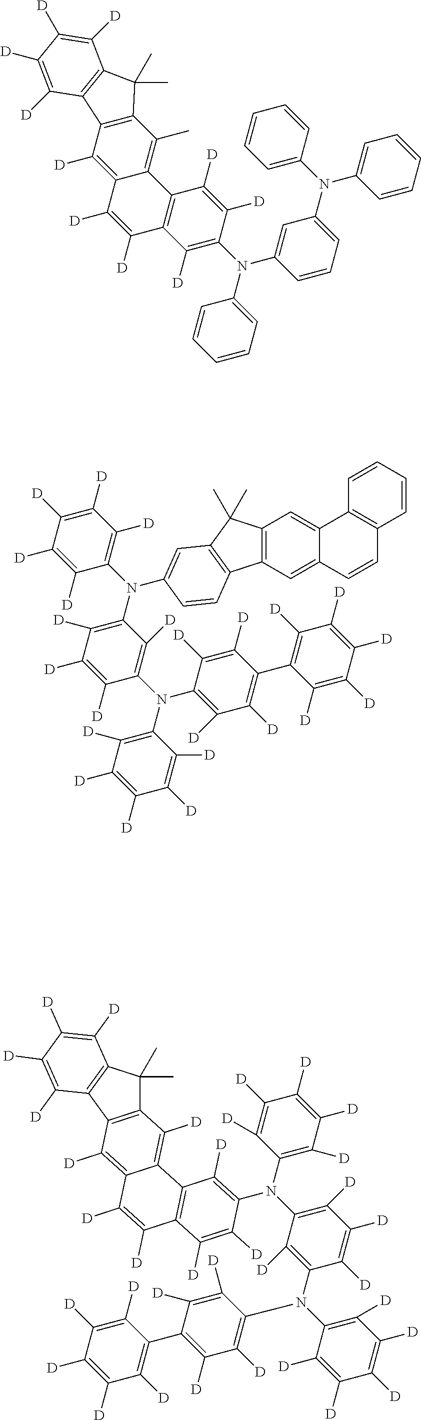

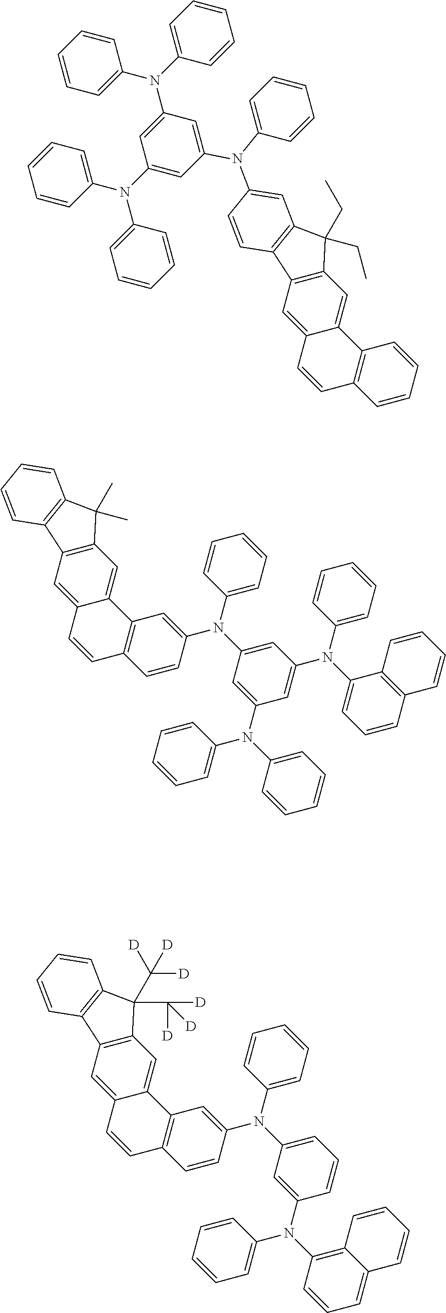 [SMILES: CCC1(CC)c2cc(N(c3ccccc3)c3cc(N(c4ccccc4)c4ccccc4)cc(N(c4ccccc4)c4ccccc4)c3)ccc2-c2cc3ccc4ccccc4c3cc21 ; CC1(C)c2ccccc2-c2cc3ccc4ccc(N(c5ccccc5)c5cc(N(c6ccccc6)c6ccccc6)cc(N(c6ccccc6)c6cccc7ccccc67)c5)cc4c3cc21 ; [2H]C([2H])([2H])C1(C([2H])([2H])[2H])c2ccccc2-c2cc3ccc4ccc(N(c5ccccc5)c5cccc(N(c6ccccc6)c6cccc7ccccc67)c5)cc4c3cc21]

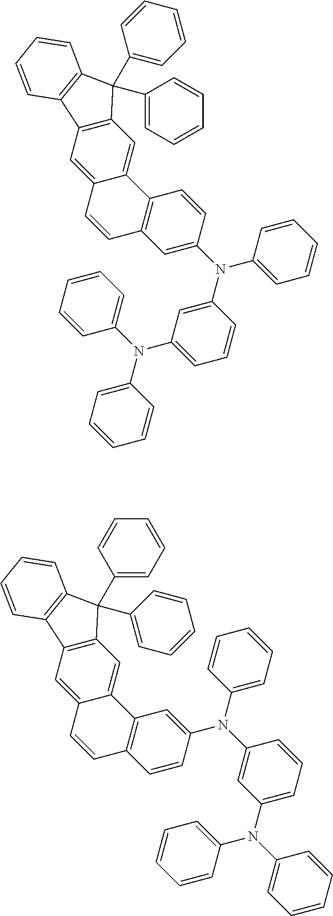 [SMILES: c1ccc(N(c2ccccc2)c2cccc(N(c3ccccc3)c3ccc4c(ccc5cc6c(cc54)C(c4ccccc4)(c4ccccc4)c4ccccc4-6)c3)c2)cc1 ; c1ccc(N(c2ccccc2)c2cccc(N(c3ccccc3)c3ccc4ccc5cc6c(cc5c4c3)C(c3ccccc3)(c3ccccc3)c3ccccc3-6)c2)cc1]

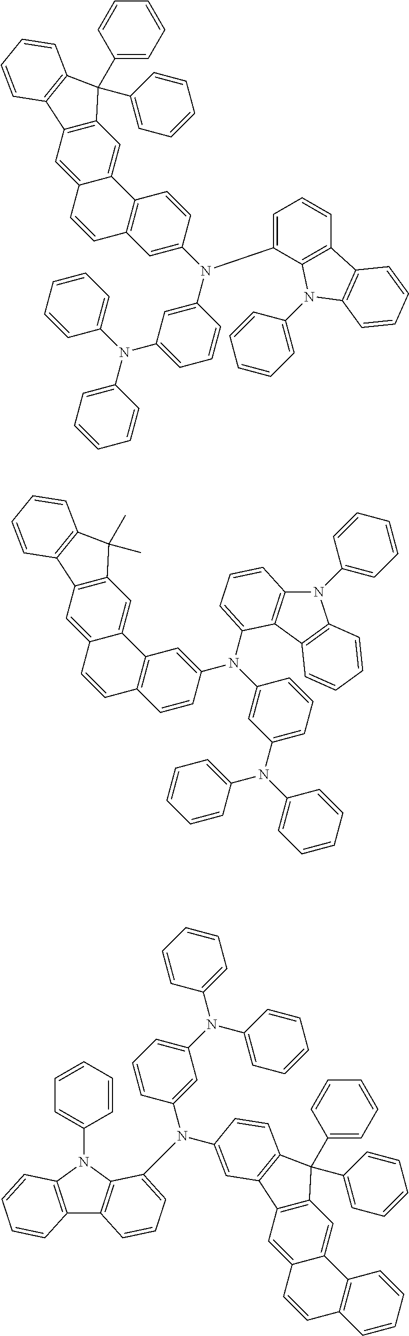 [SMILES: c1ccc(N(c2ccccc2)c2cccc(N(c3ccc4c(ccc5cc6c(cc54)C(c4ccccc4)(c4ccccc4)c4ccccc4-6)c3)c3cccc4c5ccccc5n(-c5ccccc5)c34)c2)cc1 ; CC1(C)c2ccccc2-c2cc3ccc4ccc(N(c5cccc(N(c6ccccc6)c6ccccc6)c5)c5cccc6c5c5ccccc5n6-c5ccccc5)cc4c3cc21 ; c1ccc(N(c2ccccc2)c2cccc(N(c3ccc4c(c3)-c3cc5ccc6ccccc6c5cc3C4(c3ccccc3)c3ccccc3)c3cccc4c5ccccc5n(-c5ccccc5)c34)c2)cc1]

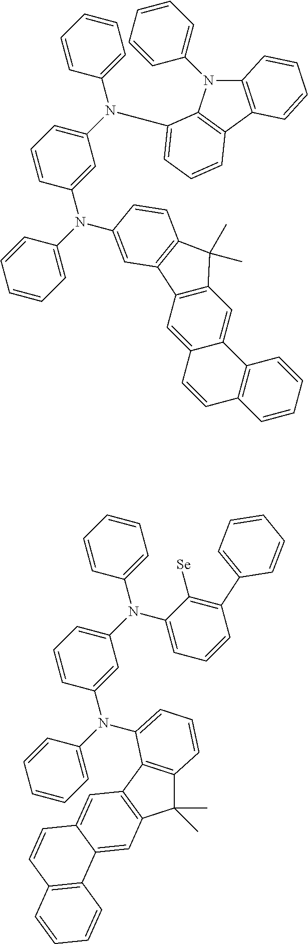

The organic electroluminescent compound according to the present disclosure may be prepared by synthetic methods known to those skilled in the art. For example, organic electroluminescent compound according to the present disclosure may be prepared by referring to the following Reaction Schemes 1 to 4, but is not limited thereto.

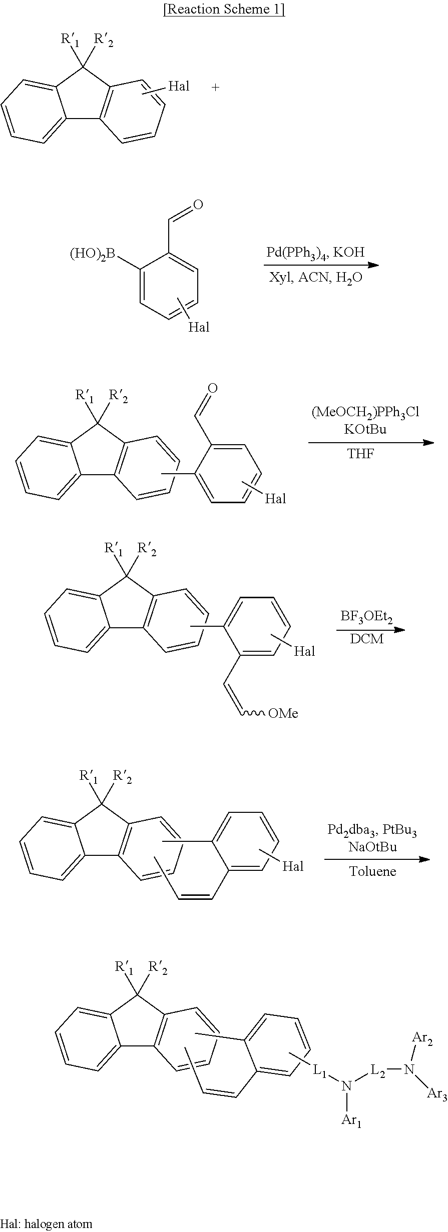

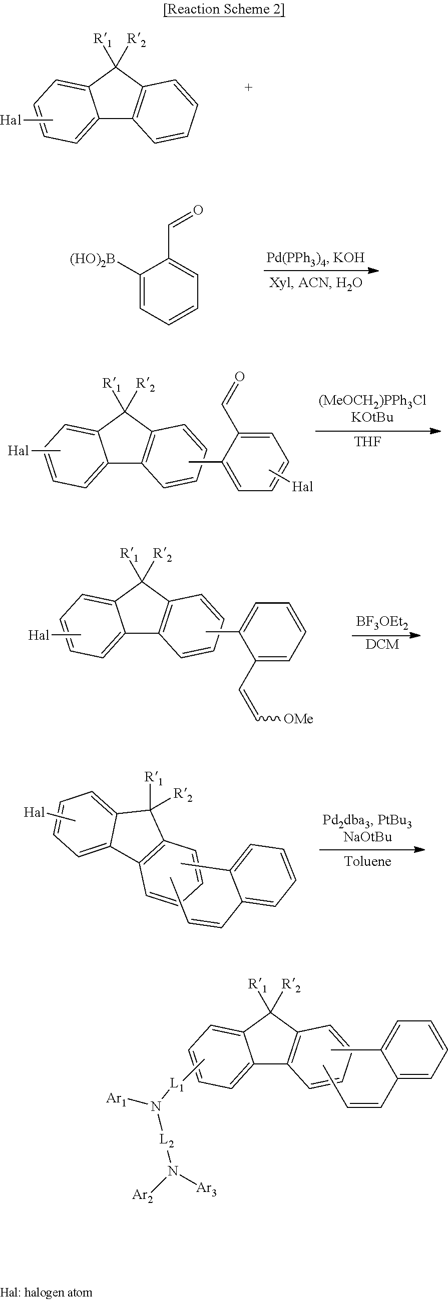

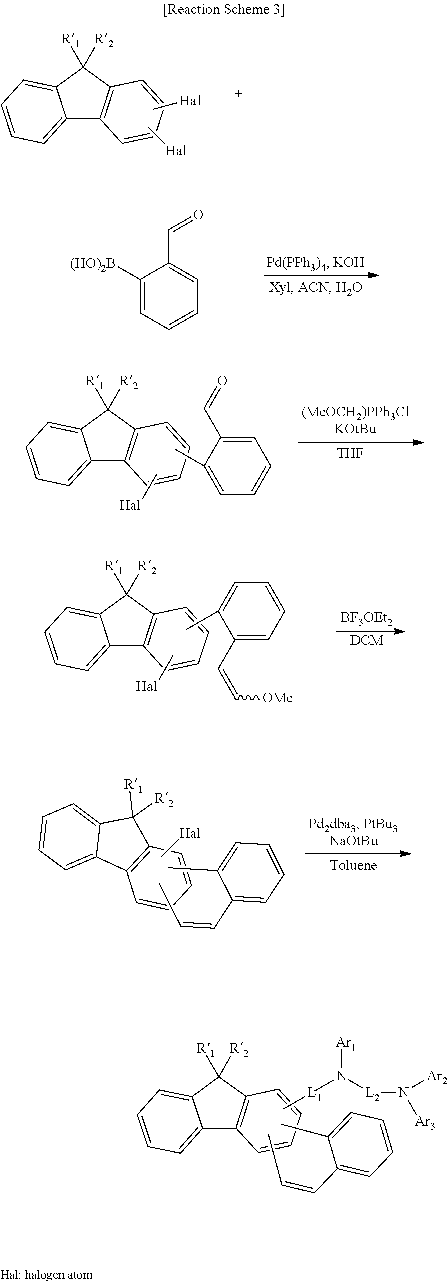

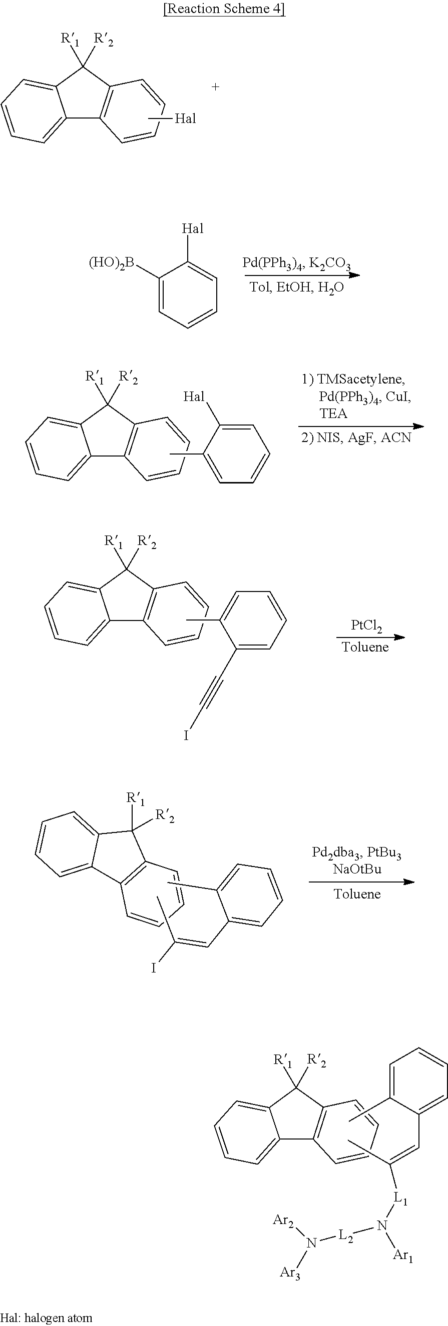

In Reaction Schemes 1 to 4, R′₁, R′₂, L₁, L₂, and Ar₁ to Ar₃ are as defined in formula 1.

Although illustrative synthesis examples of the compound represented by formula 1 are described above, one skilled in the art will be able to readily understand that all of them are based on a Buchwald-Hartwig cross coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN1 substitution reaction, an SN2 substitution reaction, a Phosphine-mediated reductive cyclization reaction, etc., and the above reactions proceed even when substituents defined in formula 1 other than the substituents specified in the specific synthesis examples are bonded.

Host compounds which may be used in combination with the compound of the present disclosure may be exemplified as a compound represented by any one of the following formulas 11 to 15, but are not limited thereto.

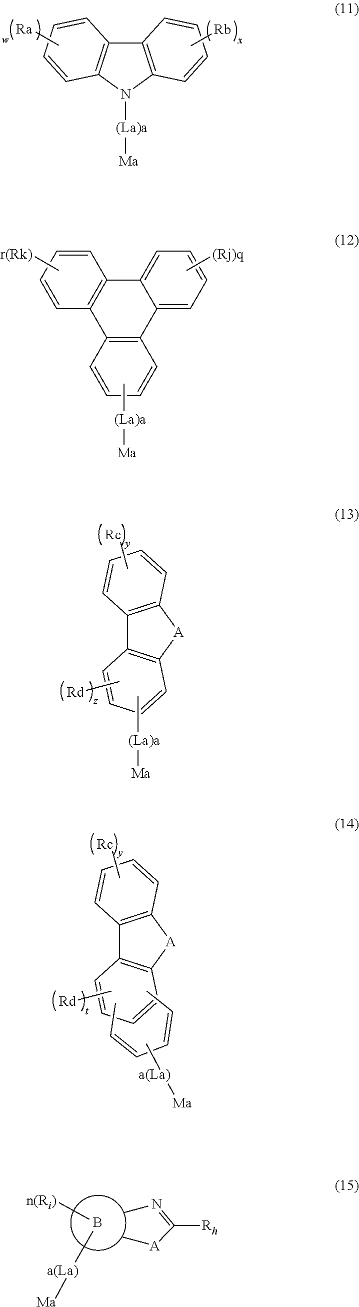

In formulas 11 to 15,
Ma represents a substituted or unsubstituted (C6-C30)aryl, a substituted or unsubstituted mono- or di- (C6-C30)arylamino, a substituted or unsubstituted mono- or di- (3- to 30-membered)heteroarylamino, a substituted or unsubstituted (C6-C30)aryl(3- to 30-membered)heteroarylamino, or a substituted or unsubstituted (3- to 30-membered)heteroaryl;
La represents a single bond, a substituted or unsubstituted (C6-C30)arylene, or a substituted or unsubstituted (3- to 30-membered)heteroarylene;
A represents S, O, N(Re), C(Rf)(Rg), Te, or Se;
ring B represents a naphthalene ring or a phenanthrene ring;
Ra to Rd and Rh to Rk, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di- (C1-C30)alkylamino, a substituted or unsubstituted (C1-C30)alkyl(C6-C30)arylamino, or a substituted or unsubstituted mono- or di-(C6-C30)arylamino; or may be linked to an adjacent substituent(s) to form a ring(s);
Re to Rg, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30)arylamino; and Rf and Rg may be linked to each other to form a ring(s);
w to y, q, and r, each independently, represent an integer of 1 to 4; z represents an integer of 1 to 3; a and t, each independently, represent an integer of 1 or 2; and n represents an integer of 1 to 9; and each of Ra to each of Rd, each of R₁ to each of Rk, and each of La may be the same as or different from each other; and
the heteroaryl(ene) contains at least one heteroatom selected from B, N, O, S, Si, P, Te, and Se.

According to one embodiment of the present disclosure, Ma represents a substituted or unsubstituted phenyl, a substituted or unsubstituted pyridyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted benzoquinazolinyl, a substituted or unsubstituted benzoquinoxalinyl, a substituted or unsubstituted dibenzofuranyl, a substituted or unsubstituted dibenzothiophenyl, a substituted or unsubstituted dibenzotelluriumyl, a substituted or unsubstituted benzofuranopyrimidinyl, a substituted or unsubstituted benzothienopyrimidinyl, a substituted or unsubstituted benzoxazolyl, etc., in which the substituents thereof, each independently, may be any one selected from the group consisting of a substituted or unsubstituted phenyl, a substituted or unsubstituted naphthyl, a biphenyl, a phenanthrenyl, a terphenyl, a chrysenyl, a benzo[c]phenanthryl, a triphenylenyl, a dimethylfluorenyl, a diphenylfluorenyl, a dimethylbenzofluorenyl, a spirobifluorenyl, a pyridyl, a pyrimidinyl, a triazinyl substituted with a phenyl(s), a dibenzofuranyl unsubstituted or substituted with at least one of deuterium and a phenyl(s), a dibenzothiophenyl, a carbazolyl unsubstituted or substituted with at least one of a phenyl(s) and a biphenyl(s), a dibenzotelluriumyl, a dibenzoselenophenyl, a benzonaphthofuranyl, a benzonaphthothiophenyl, a phenanthrooxazolyl substituted with a phenyl(s), a triphenylsilyl, and a triphenylgermanyl, or a combination thereof, in which the substituent(s) of the substituted phenyl may be at least one of deuterium, a cyano, a fluoro, a methyl, a naphthyl, a carbazolyl, a triphenylsilyl, a triphenylgermanyl, and a dibenzotelluriumyl, and the substituent(s) of the substituted naphthyl may be at least one of a phenyl, a biphenyl, and a chrysenyl.

According to one embodiment of the present disclosure, La represents a single bond; a (C6-C25)arylene unsubstituted or substituted with at least one of deuterium, a (C1-C30)alkyl(s) and a (C6-C30)aryl(s); or a (5- to 25-membered)heteroarylene unsubstituted or substituted with a (C6-C30)aryl(s). For example, La may be a single bond; a phenylene unsubstituted or substituted with at least one of deuterium and a phenyl(s); a naphthylene unsubstituted or substituted with deuterium; a biphenylene unsubstituted or substituted with a phenyl(s); a phenanthrenylene; a terphenylene; a dimethylfluorenylene; a pyridylene; a dibenzofuranylene; a dibenzothiophenylene; a quinoxalinylene substituted with a phenyl(s); or a quinazolinylene, etc.

According to one embodiment of the present disclosure, Ra to Rd and Rh to Rk, each independently, represent hydrogen, deuterium, a cyano, a substituted or unsubstituted (C1-C20)alkyl, a substituted or unsubstituted (C6-C30)aryl, or a substituted or unsubstituted (5- to 20-membered)heteroaryl; or may be linked to an adjacent substituent(s) to form a substituted or unsubstituted, mono- or polycyclic, (3- to 30-membered) alicyclic or aromatic ring, or the combination thereof. For example, Ra to Rd and R₁ to Rk, each independently, may be hydrogen, deuterium, a cyano, a methyl, a phenyl, a naphthyl, a biphenyl unsubstituted or substituted with deuterium, a phenylnaphthyl, a naphthylphenyl, a phenanthrenyl, a dimethylfluorenyl, a dibenzofuranyl unsubstituted or substituted with a phenyl(s), a dibenzothiophenyl unsubstituted or substituted with a phenyl(s), or a carbazolyl unsubstituted or substituted with a phenyl(s), etc.; or may be linked to an adjacent substituent(s) to form a benzene ring, a benzofuran ring unsubstituted or substituted with a phenyl(s), or a benzothiophene ring unsubstituted or substituted with a phenyl(s), etc. For example, Rh may be a substituted phenyl, a naphthyl unsubstituted or substituted with a diphenyltriazinyl(s), a biphenyl, a phenanthrenyl, a dimethylfluorenyl, a pyridyl unsubstituted or substituted with a phenyl(s), a dibenzothiophenyl, a dibenzofuranyl, a phenylcarbazolyl, etc., in which the substituent(s) of the substituted phenyl may be at least one of deuterium, a cyano, a triazinyl substituted a phenyl(s), a triphenylsilyl, and a triphenylgermanyl.

According to one embodiment of the present disclosure, Re to Rg, each independently, represent an unsubstituted (C1-C20)alkyl, an unsubstituted (C6-C25)aryl, or an unsubstituted (5- to 20-membered)heteroaryl; and Rf and Rg may be linked to each other to form a substituted or unsubstituted, mono- or polycyclic, (3- to 30-membered) alicyclic or aromatic ring, or the combination thereof, in which the formed alicyclic or aromatic ring, or the combination thereof may contain at least one heteroatom selected from nitrogen, oxygen, and sulfur. For example, Re may be a phenyl or a dibenzotellurium; and Rf and Rg, each independently, may be a methyl or a phenyl, etc., or Rf and Rg may be linked to each other to form a spirofluorene ring.

The compound represented by formula 11 may be at least one selected from the group consisting of the following compounds, but is not limited thereto.

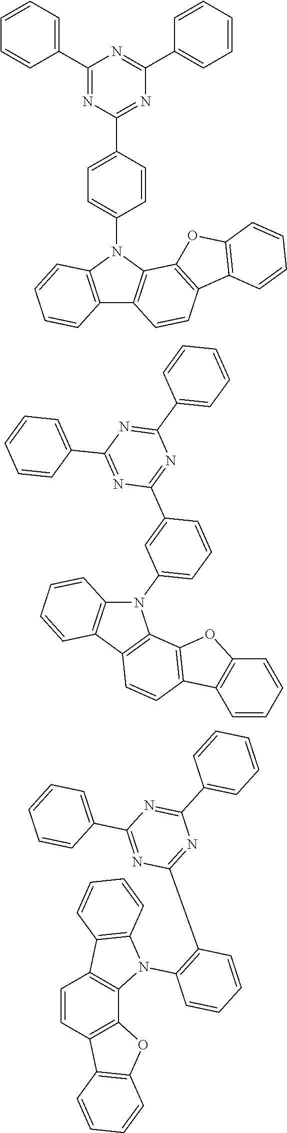

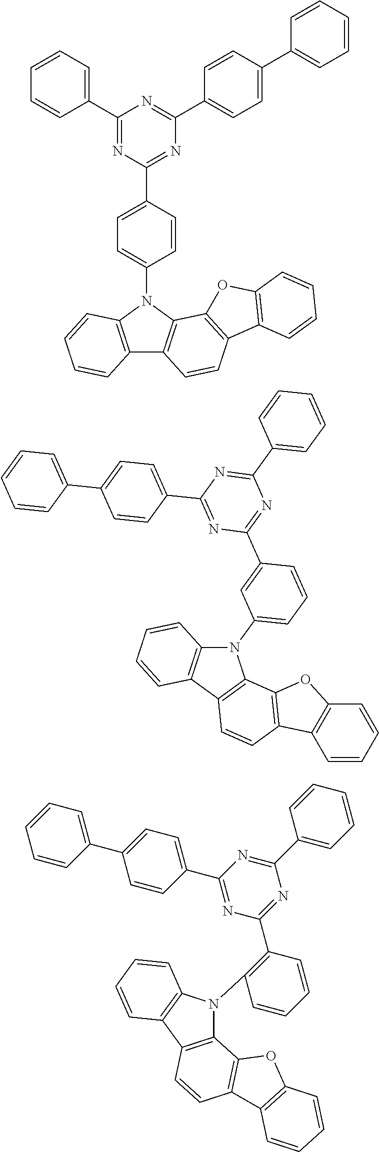

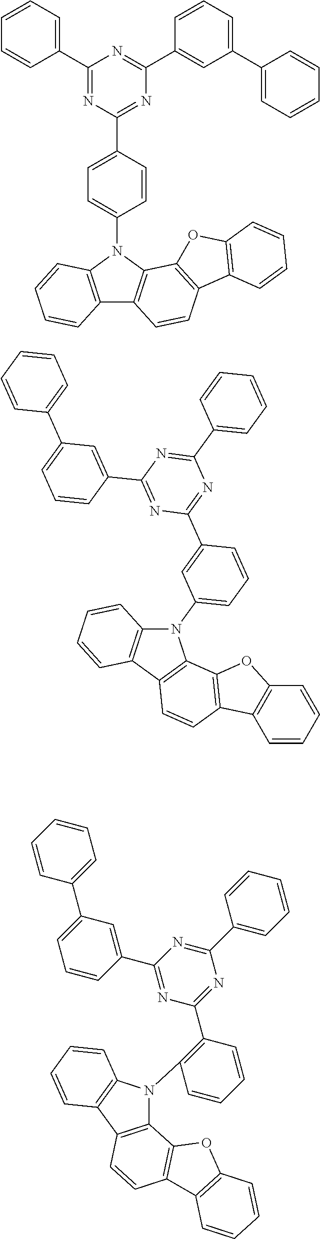

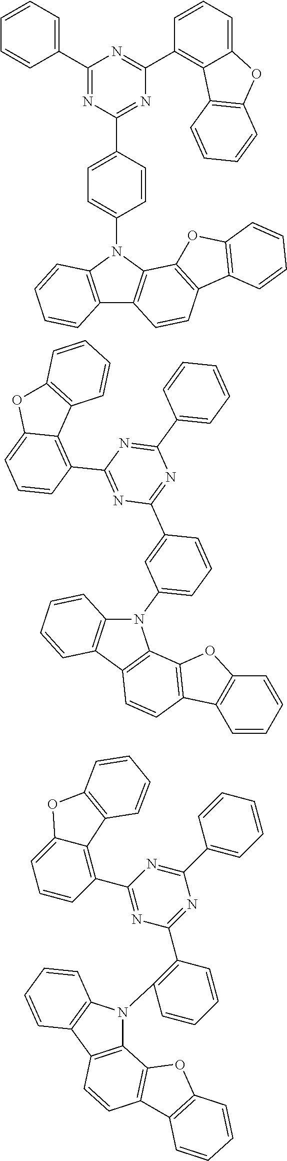

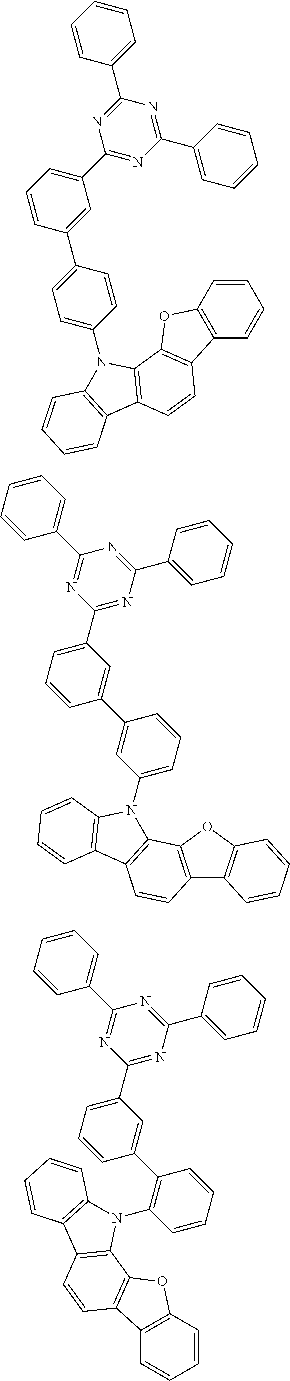

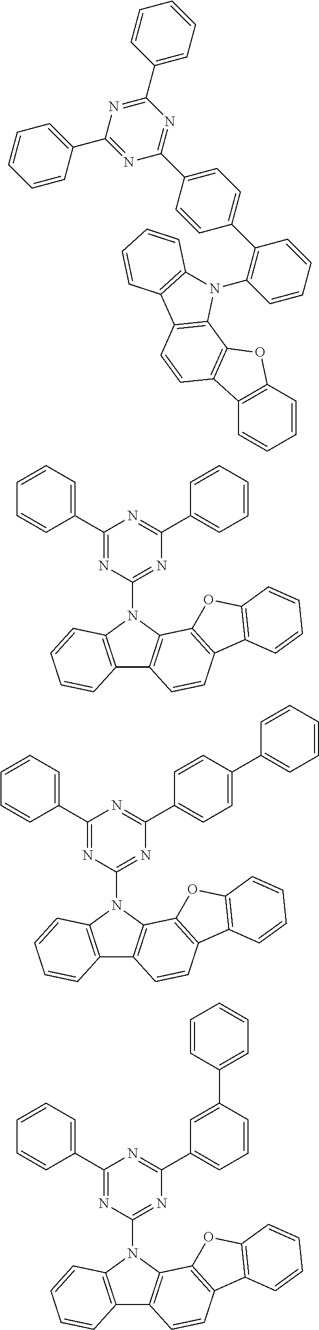

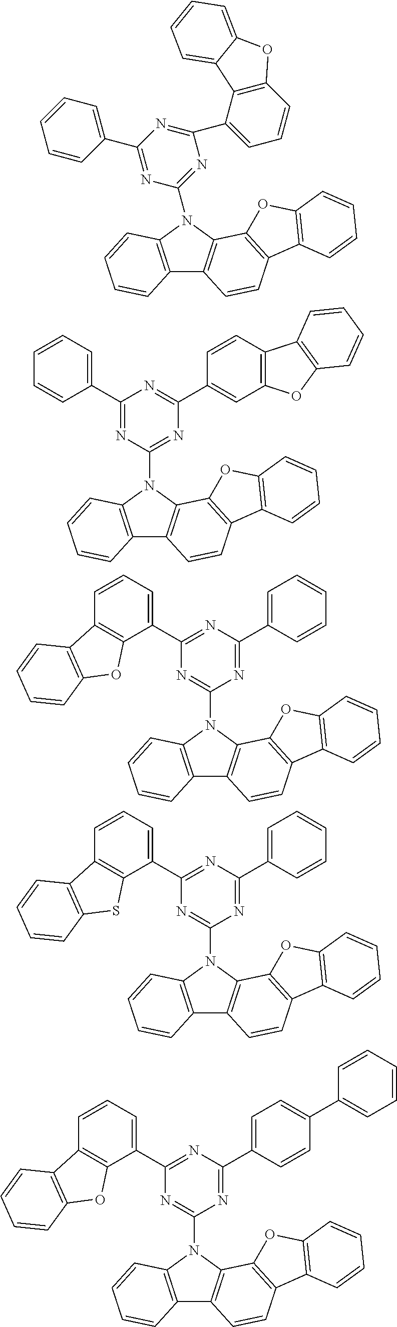

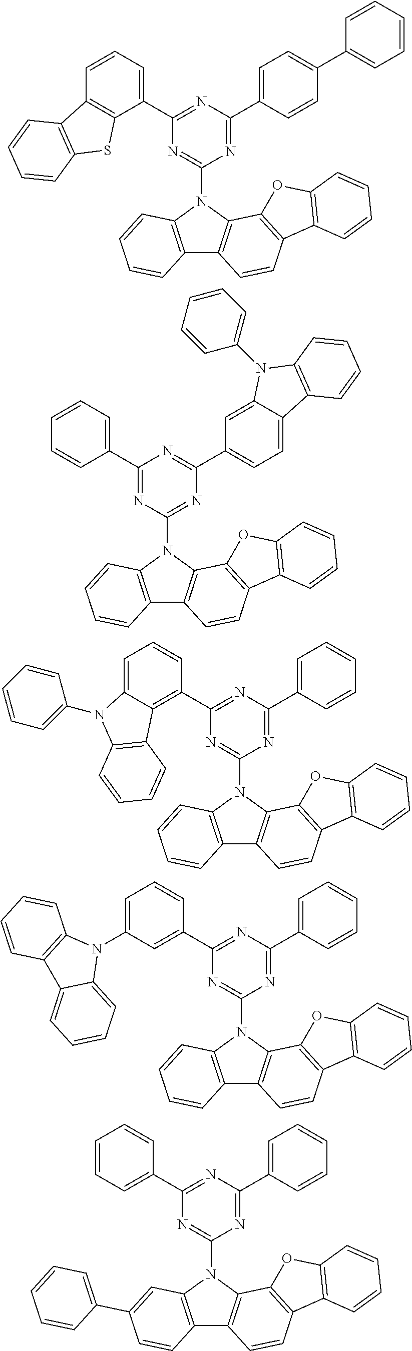

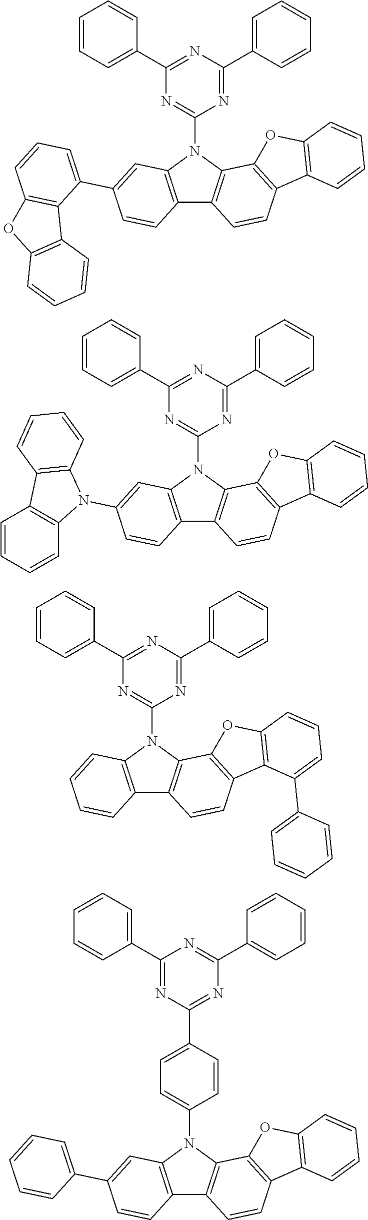

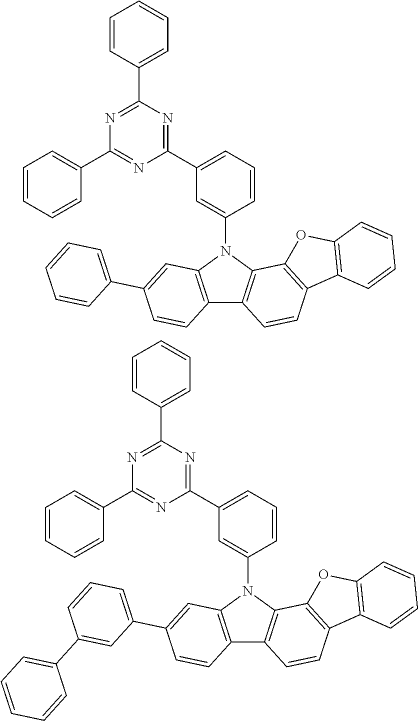

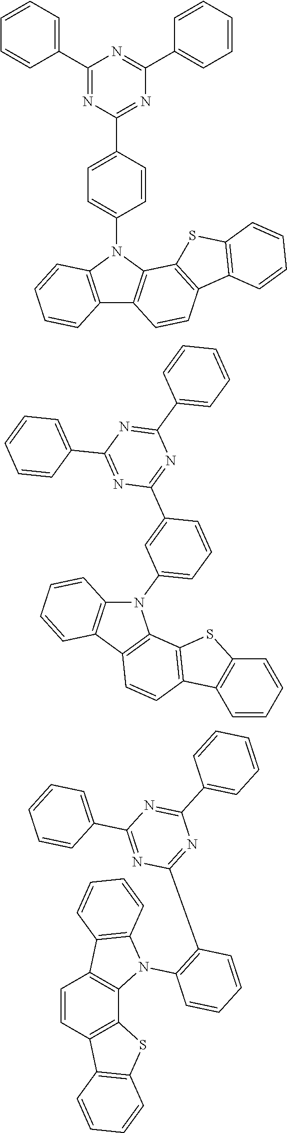

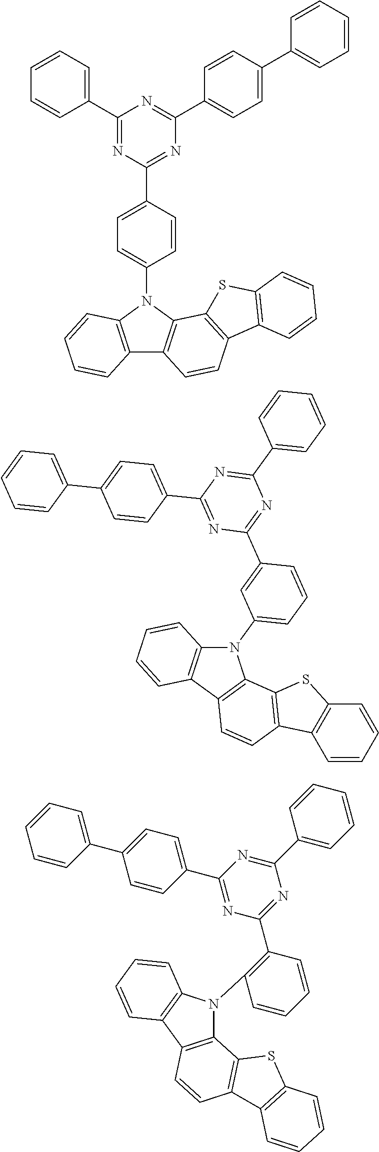

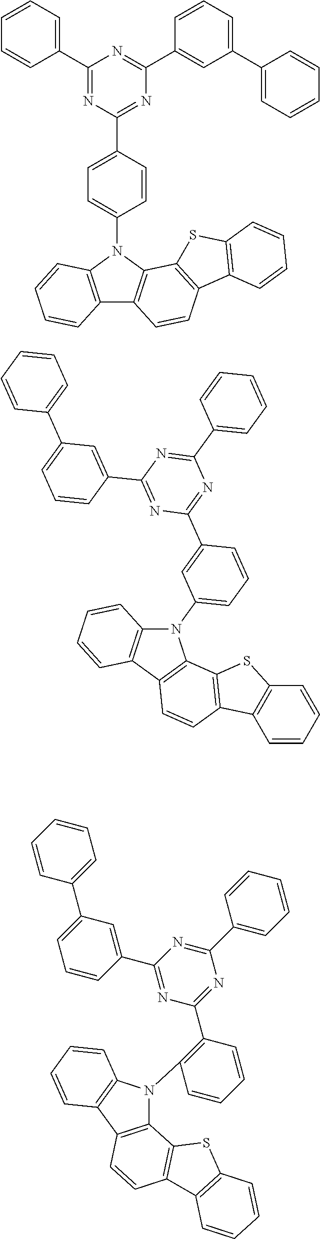

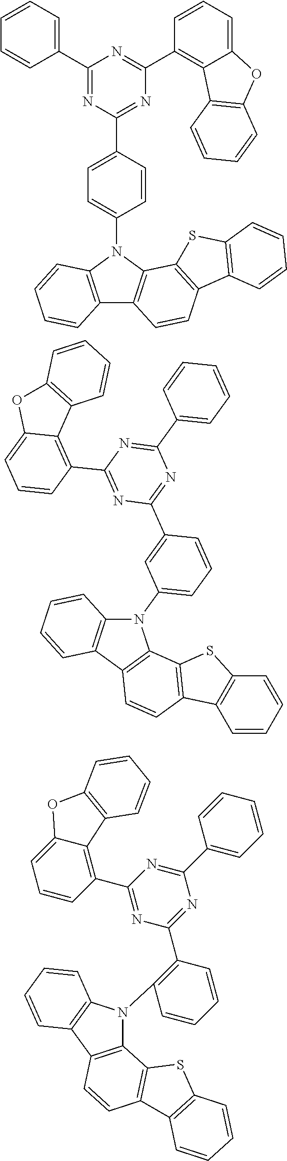

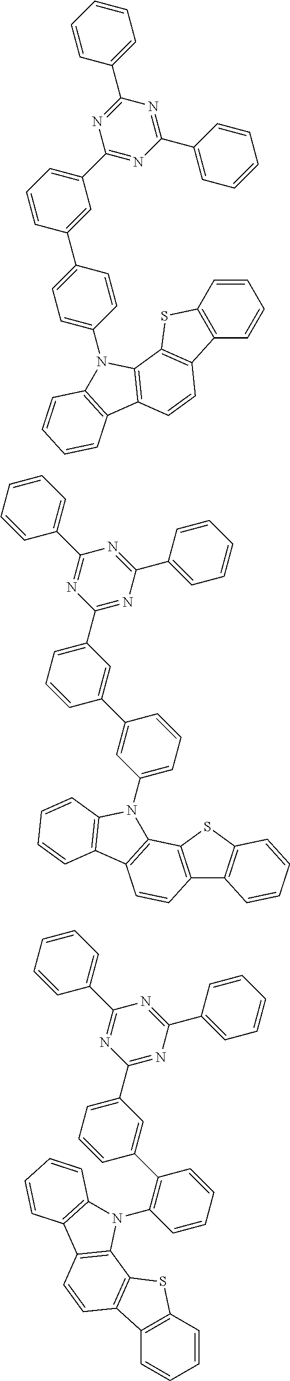

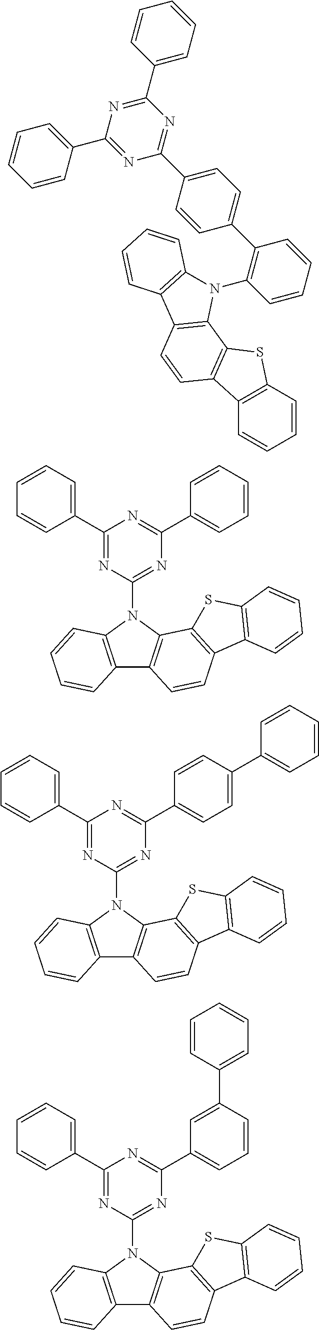

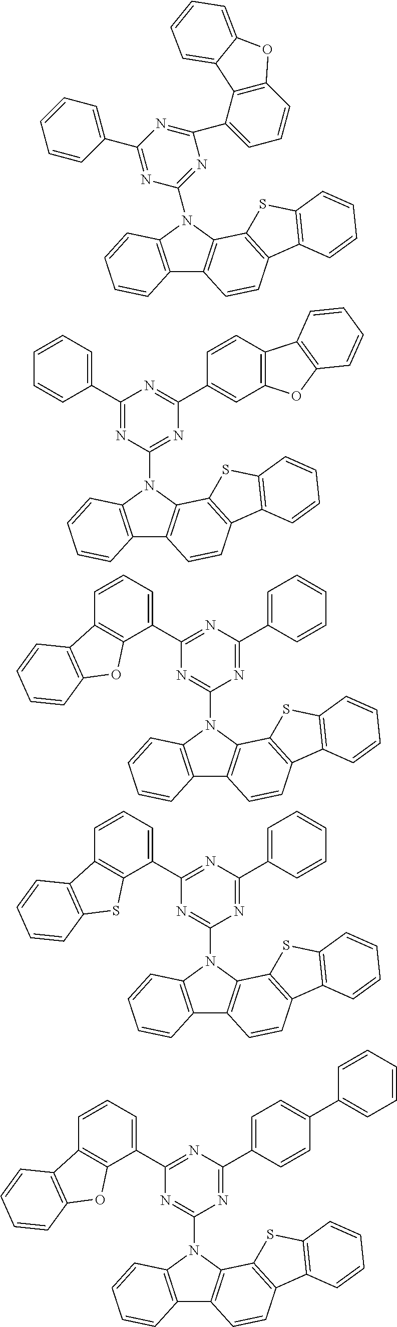

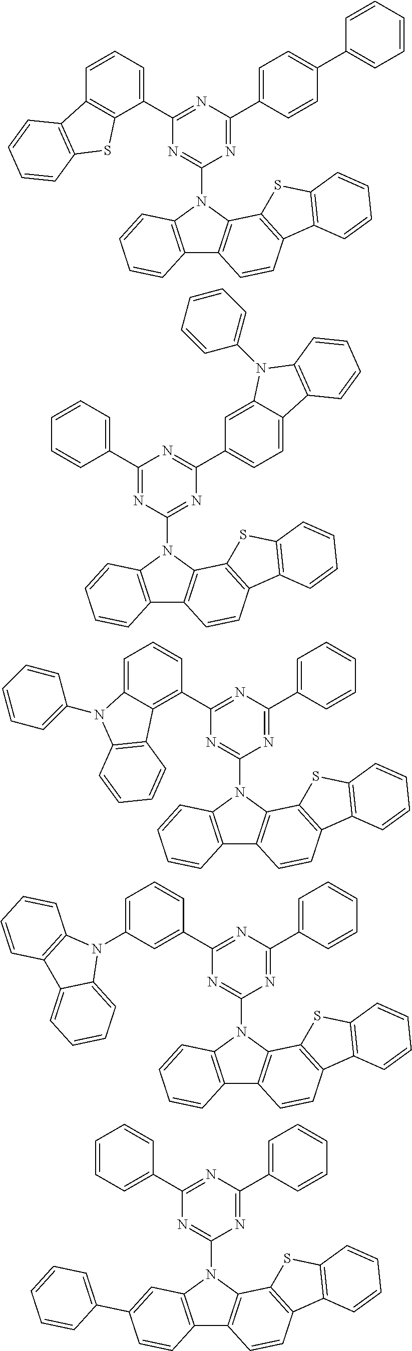

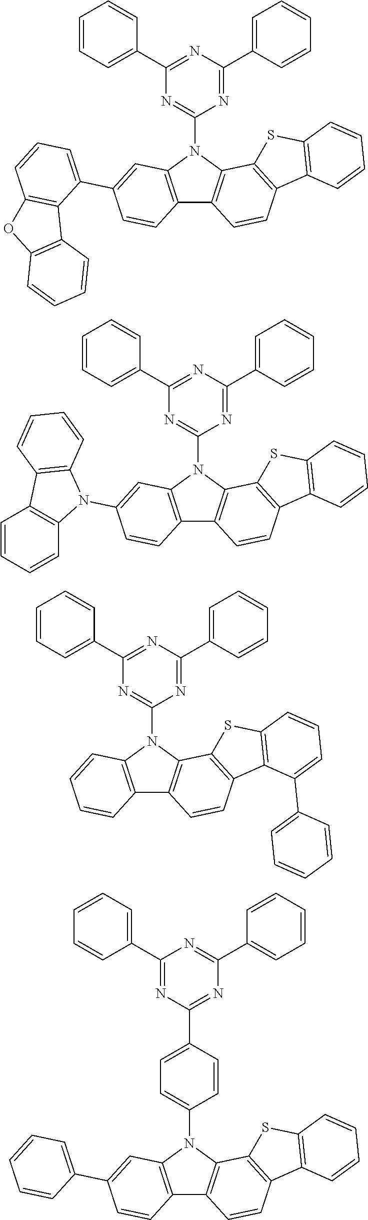

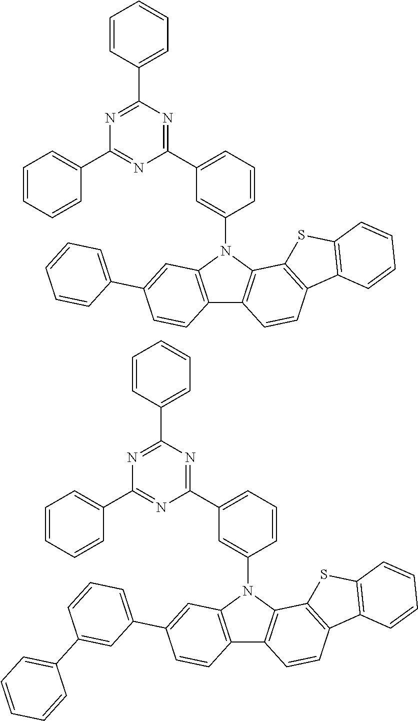

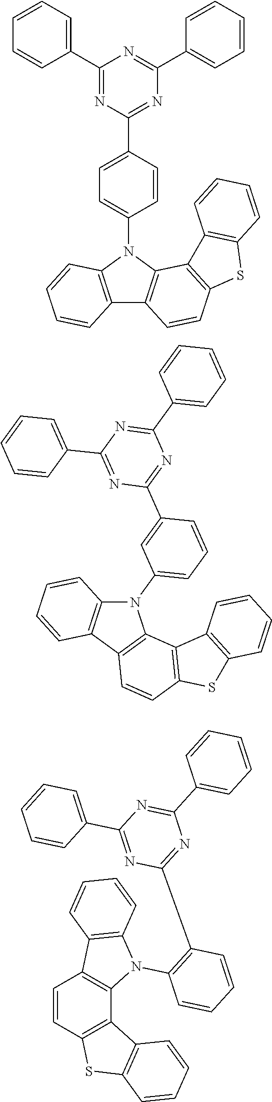

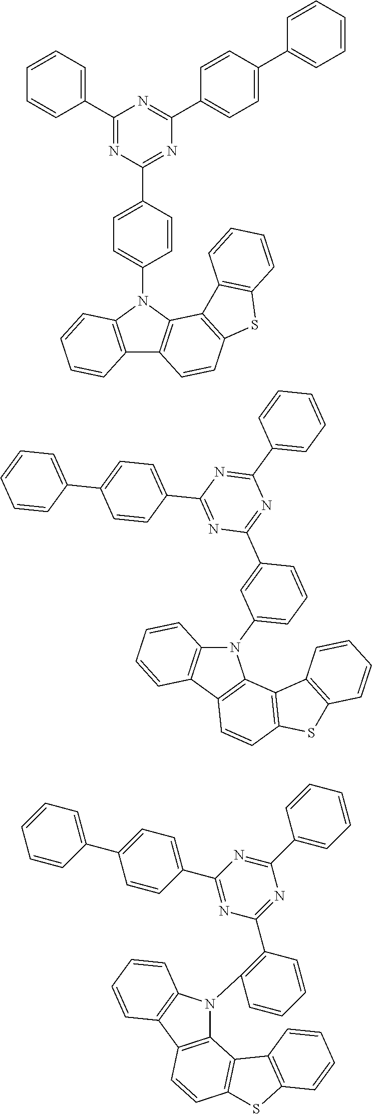 [SMILES: c1ccc(-c2ccc(-c3nc(-c4ccccc4)nc(-c4ccc(-n5c6ccccc6c6ccc7sc8ccccc8c7c65)cc4)n3)cc2)cc1 ; c1ccc(-c2ccc(-c3nc(-c4ccccc4)nc(-c4cccc(-n5c6ccccc6c6ccc7sc8ccccc8c7c65)c4)n3)cc2)cc1 ; c1ccc(-c2ccc(-c3nc(-c4ccccc4)nc(-c4ccccc4-n4c5ccccc5c5ccc6sc7ccccc7c6c54)n3)cc2)cc1]

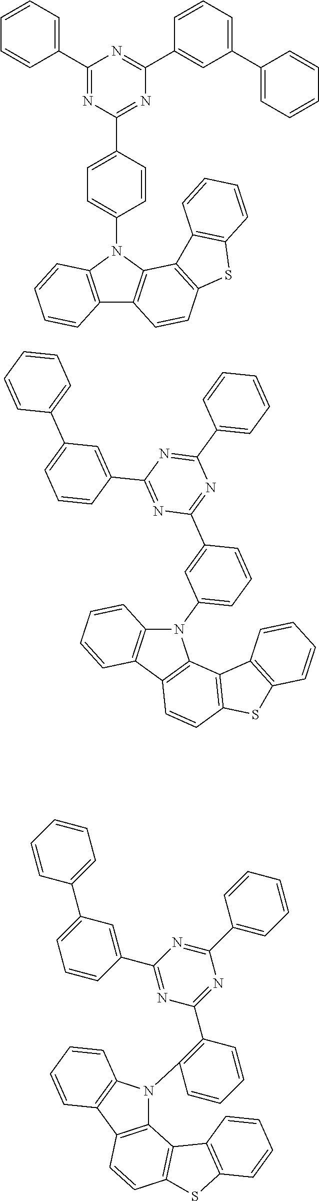

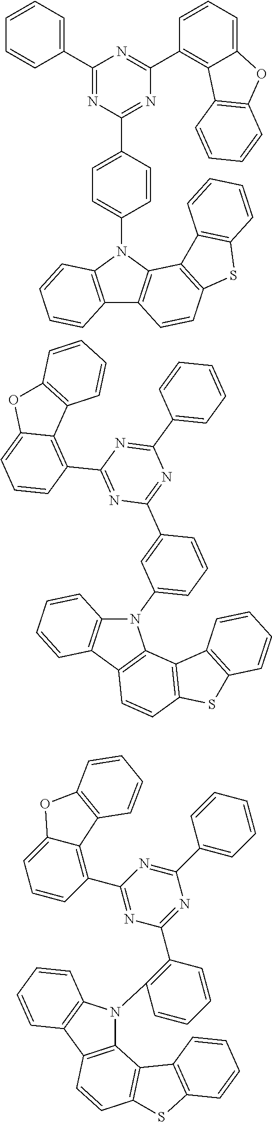

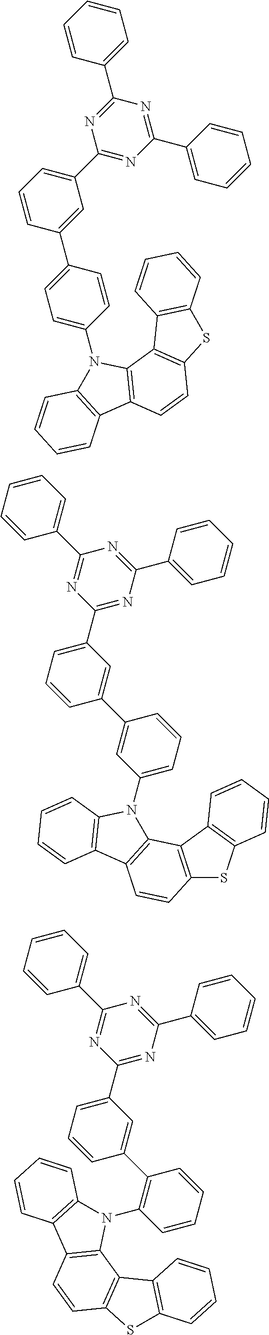

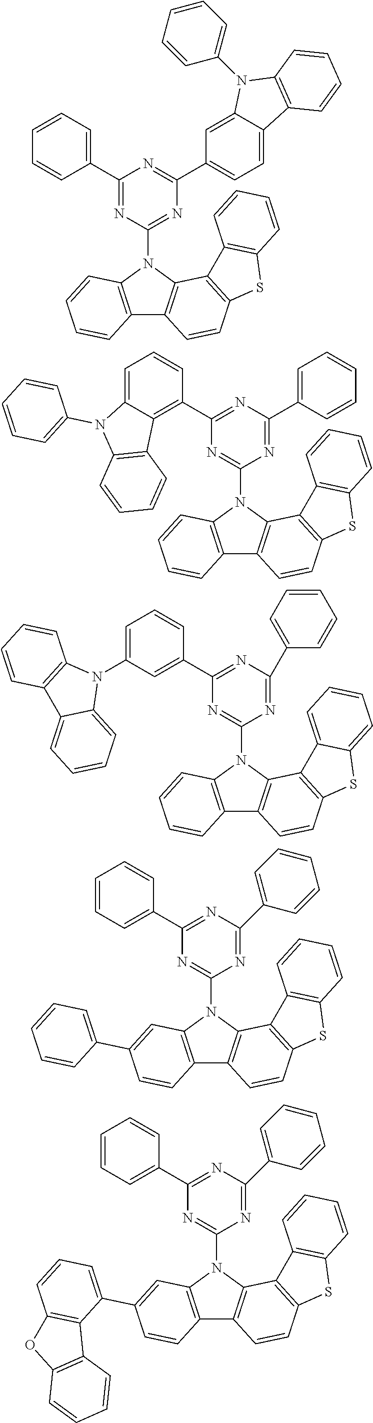

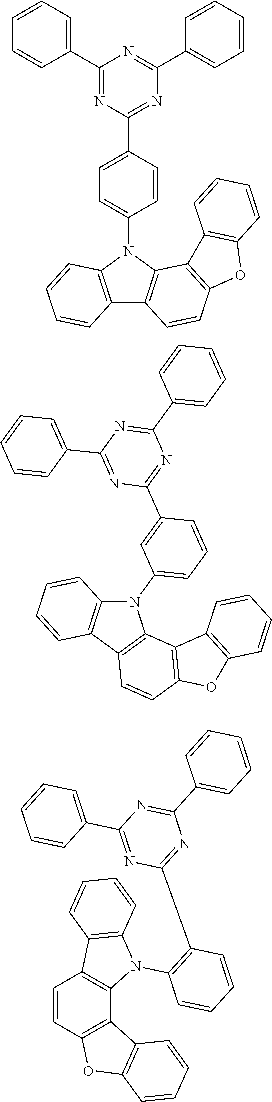

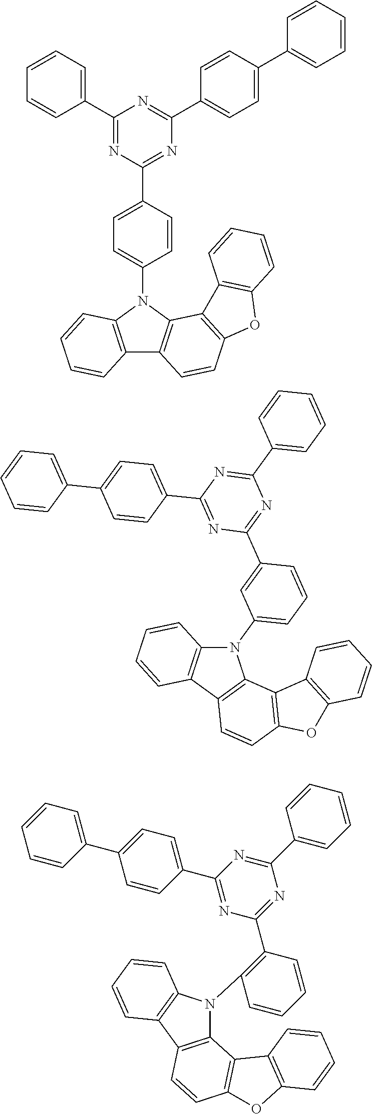 [SMILES: c1ccc(-c2ccc(-c3nc(-c4ccccc4)nc(-c4ccc(-n5c6ccccc6c6ccc7oc8ccccc8c7c65)cc4)n3)cc2)cc1 ; c1ccc(-c2ccc(-c3nc(-c4ccccc4)nc(-c4cccc(-n5c6ccccc6c6ccc7oc8ccccc8c7c65)c4)n3)cc2)cc1 ; c1ccc(-c2ccc(-c3nc(-c4ccccc4)nc(-c4ccccc4-n4c5ccccc5c5ccc6oc7ccccc7c6c54)n3)cc2)cc1]

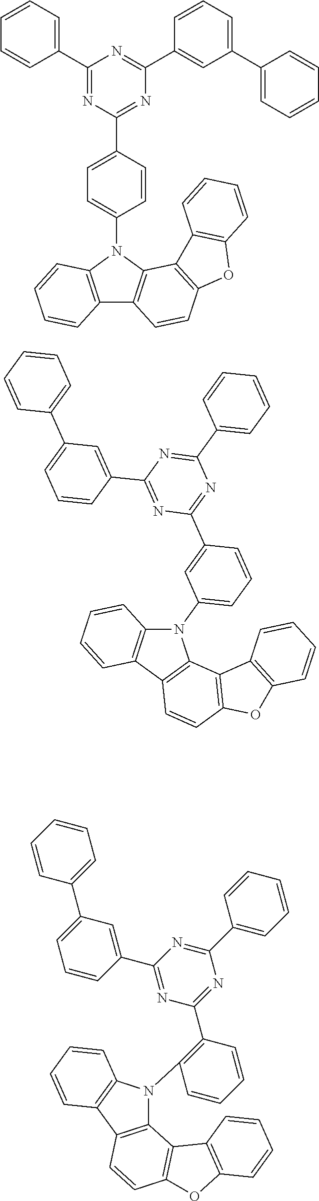

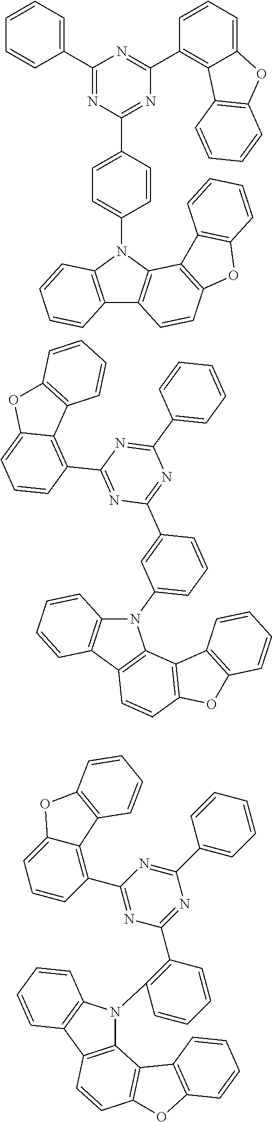

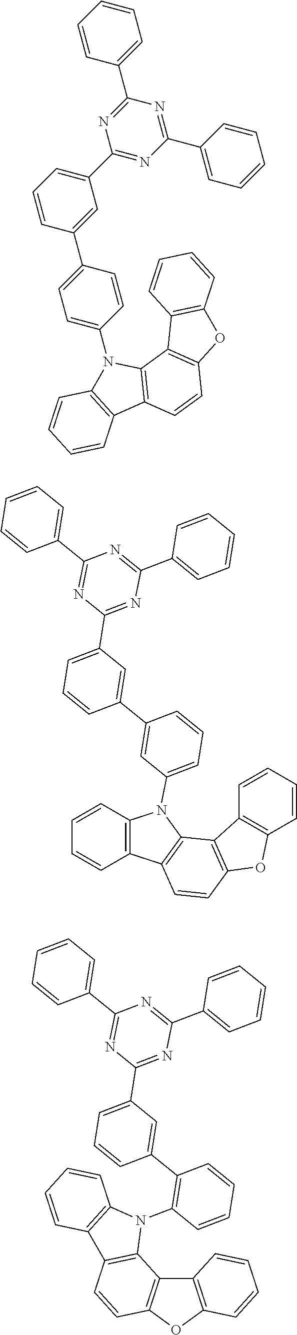 [SMILES: c1ccc(-c2nc(-c3ccccc3)nc(-c3cccc(-c4ccc(-n5c6ccccc6c6ccc7oc8ccccc8c7c65)cc4)c3)n2)cc1 ; c1ccc(-c2nc(-c3ccccc3)nc(-c3cccc(-c4cccc(-n5c6ccccc6c6ccc7oc8ccccc8c7c65)c4)c3)n2)cc1 ; c1ccc(-c2nc(-c3ccccc3)nc(-c3cccc(-c4ccccc4-n4c5ccccc5c5ccc6oc7ccccc7c6c54)c3)n2)cc1]

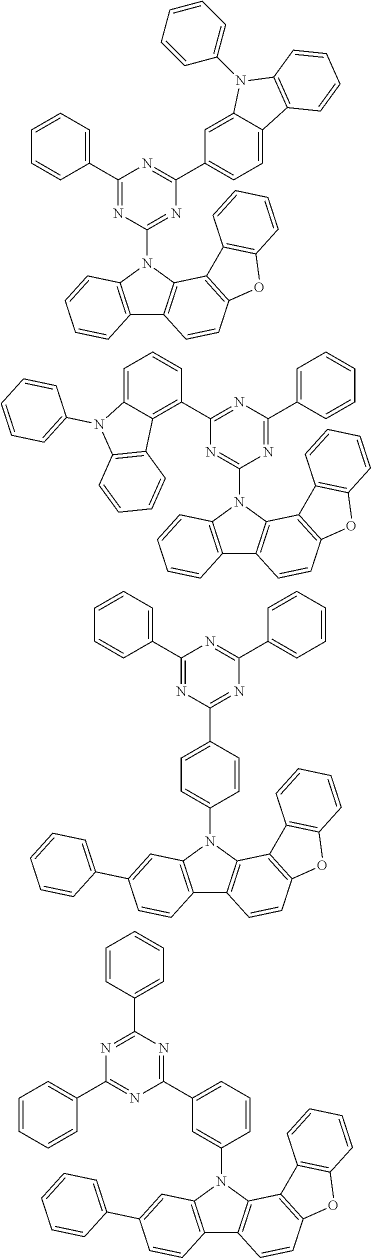

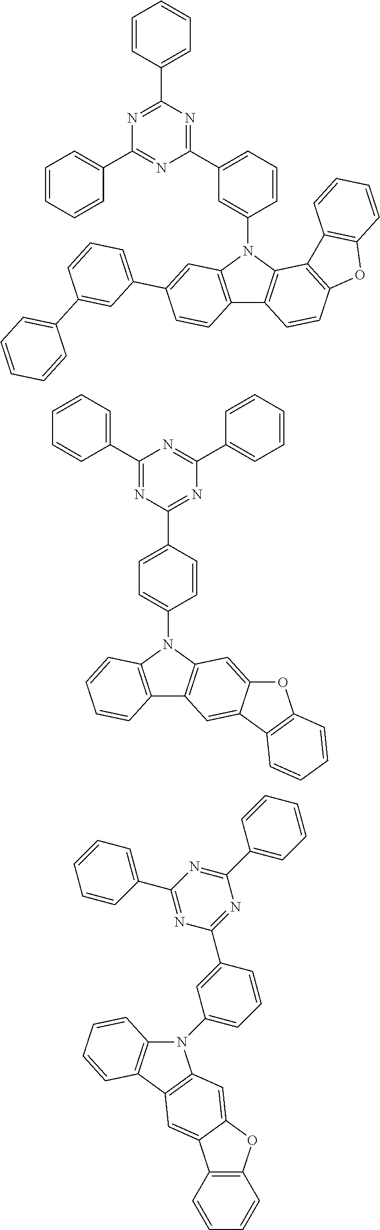

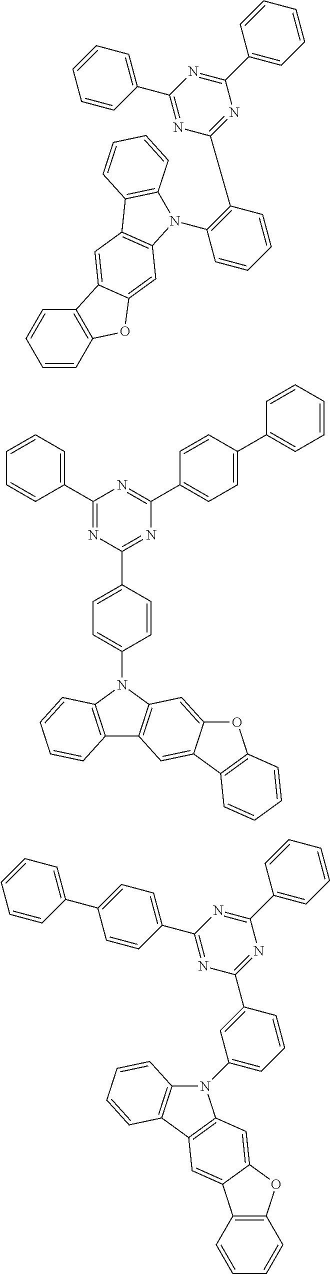

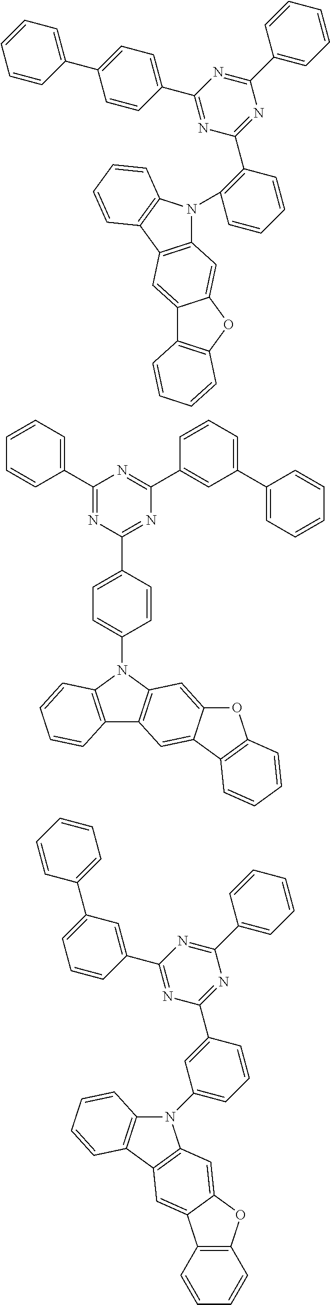

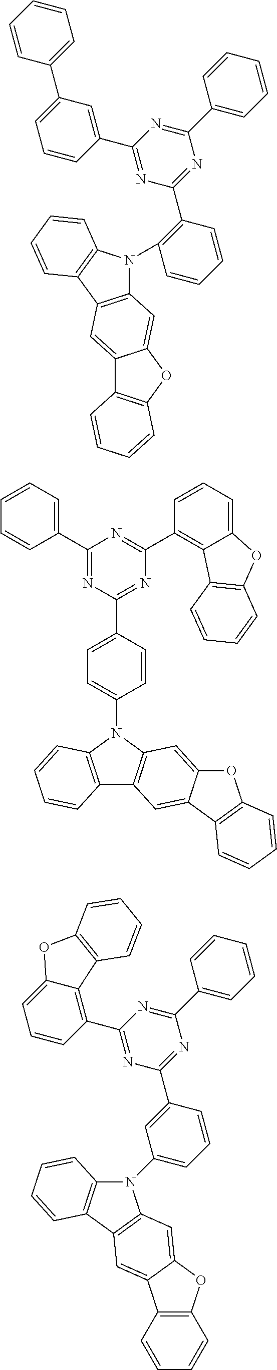 [SMILES: c1ccc(-c2cccc(-c3nc(-c4ccccc4)nc(-c4ccccc4-n4c5ccccc5c5cc6c(cc54)oc4ccccc46)n3)c2)cc1 ; c1ccc(-c2nc(-c3ccc(-n4c5ccccc5c5cc6c(cc54)oc4ccccc46)cc3)nc(-c3cccc4oc5ccccc5c34)n2)cc1 ; c1ccc(-c2nc(-c3cccc(-n4c5ccccc5c5cc6c(cc54)oc4ccccc46)c3)nc(-c3cccc4oc5ccccc5c34)n2)cc1]

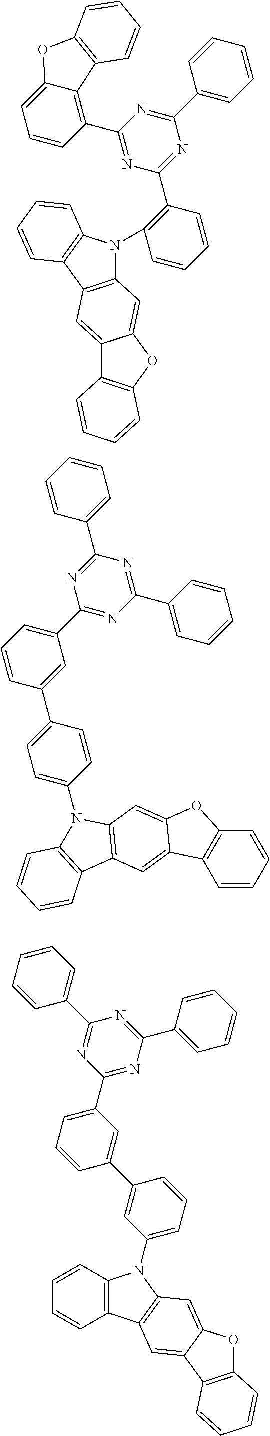

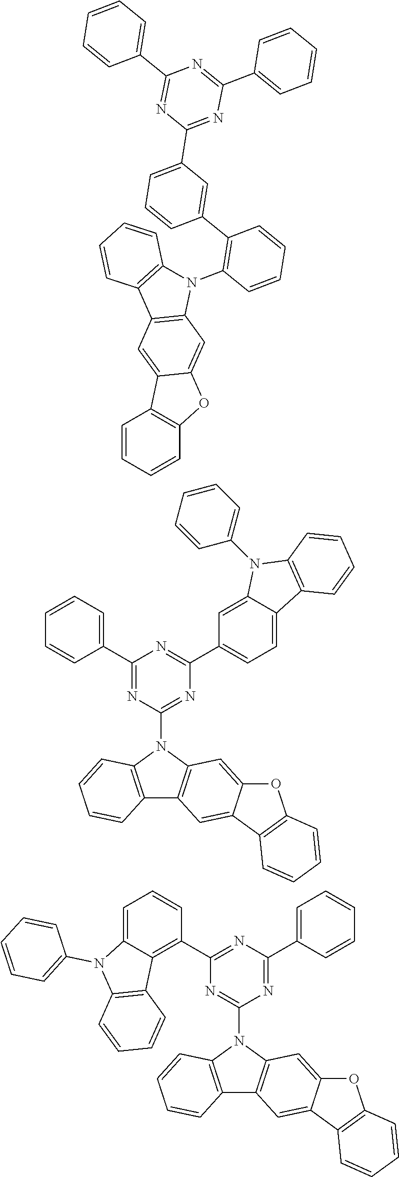

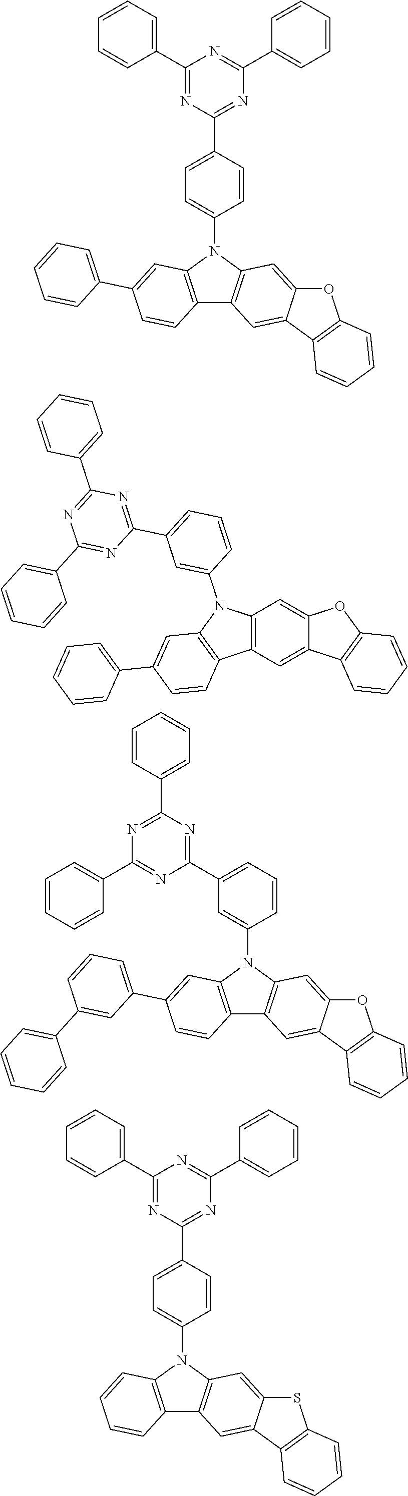

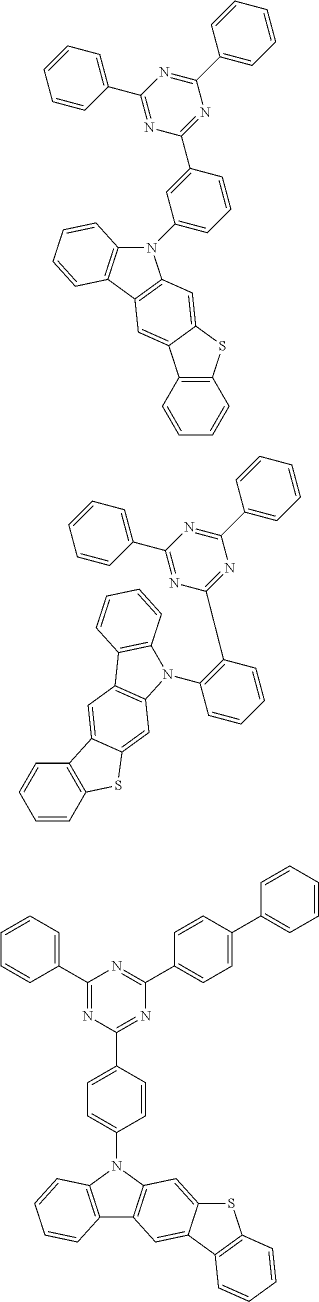 [SMILES: c1ccc(-c2nc(-c3ccccc3)nc(-c3cccc(-n4c5ccccc5c5cc6c(cc54)sc4ccccc46)c3)n2)cc1 ; c1ccc(-c2nc(-c3ccccc3)nc(-c3ccccc3-n3c4ccccc4c4cc5c(cc43)sc3ccccc35)n2)cc1 ; c1ccc(-c2ccc(-c3nc(-c4ccccc4)nc(-c4ccc(-n5c6ccccc6c6cc7c(cc65)sc5ccccc57)cc4)n3)cc2)cc1]

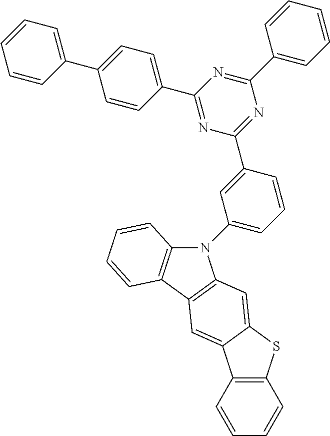 [SMILES: c1ccc(-c2ccc(-c3nc(-c4ccccc4)nc(-c4cccc(-n5c6ccccc6c6cc7c(cc65)sc5ccccc57)c4)n3)cc2)cc1]

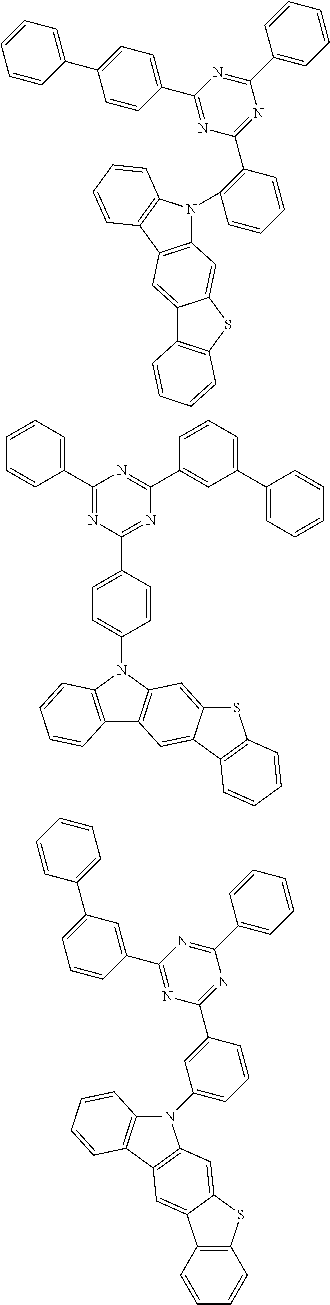

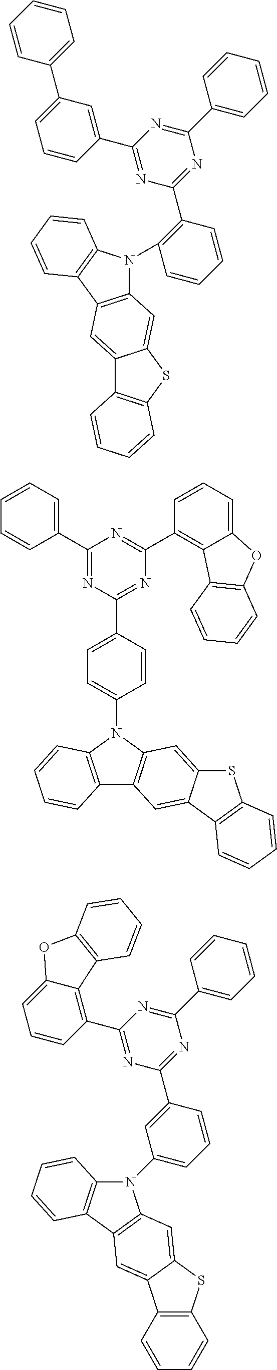 [SMILES: c1ccc(-c2cccc(-c3nc(-c4ccccc4)nc(-c4ccccc4-n4c5ccccc5c5cc6c(cc54)sc4ccccc46)n3)c2)cc1 ; c1ccc(-c2nc(-c3ccc(-n4c5ccccc5c5cc6c(cc54)sc4ccccc46)cc3)nc(-c3cccc4oc5ccccc5c34)n2)cc1 ; c1ccc(-c2nc(-c3cccc(-n4c5ccccc5c5cc6c(cc54)sc4ccccc46)c3)nc(-c3cccc4oc5ccccc5c34)n2)cc1]

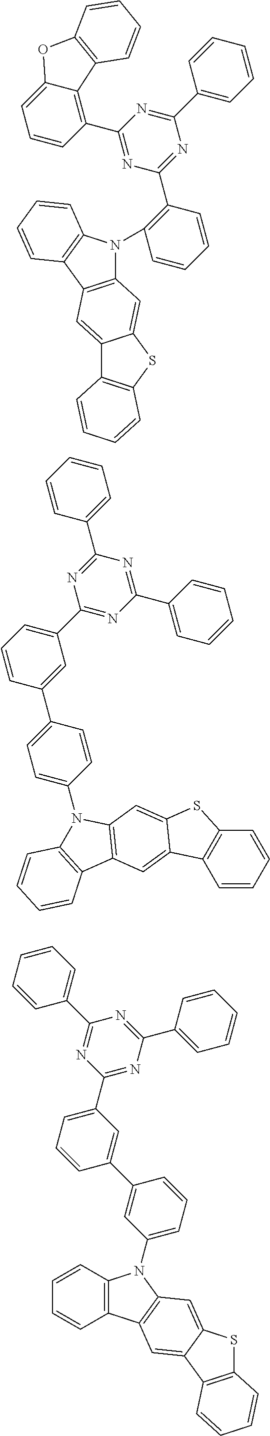

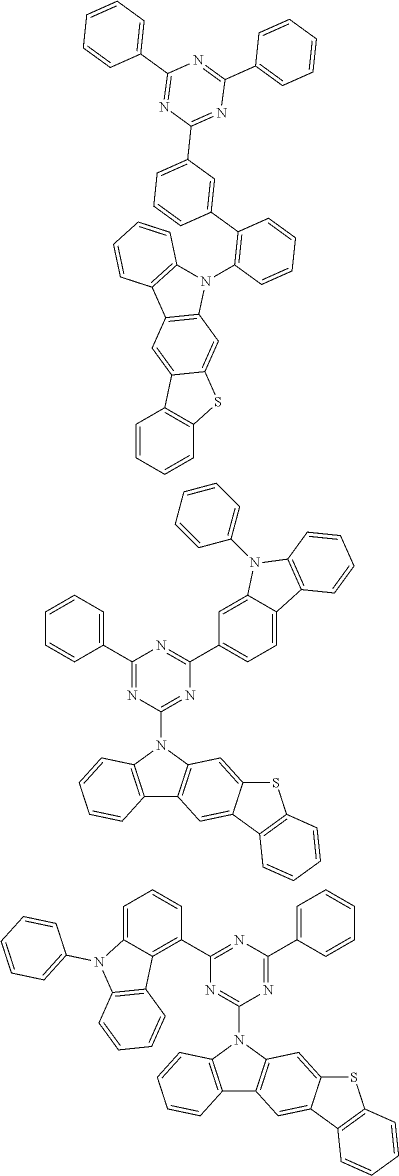

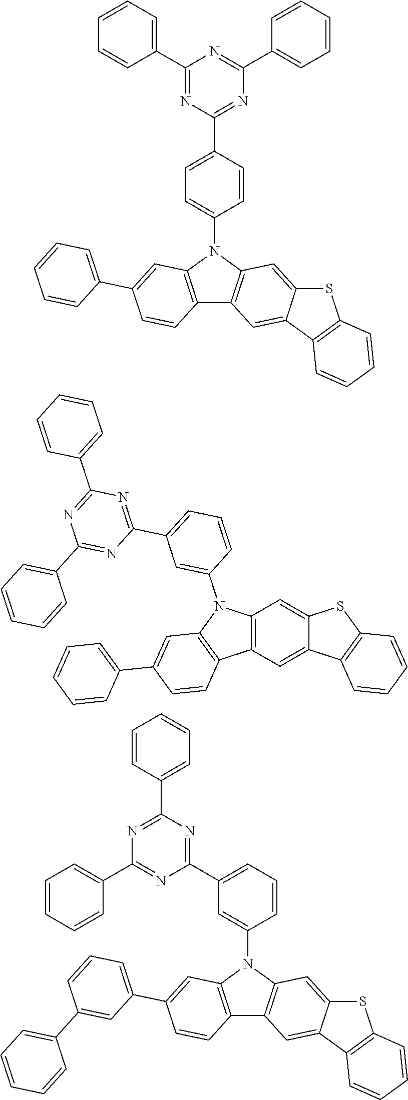

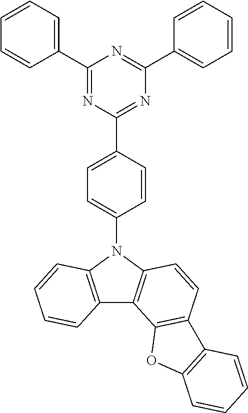 [SMILES: c1ccc(-c2nc(-c3ccccc3)nc(-c3ccc(-n4c5ccccc5c5c6oc7ccccc7c6ccc54)cc3)n2)cc1]

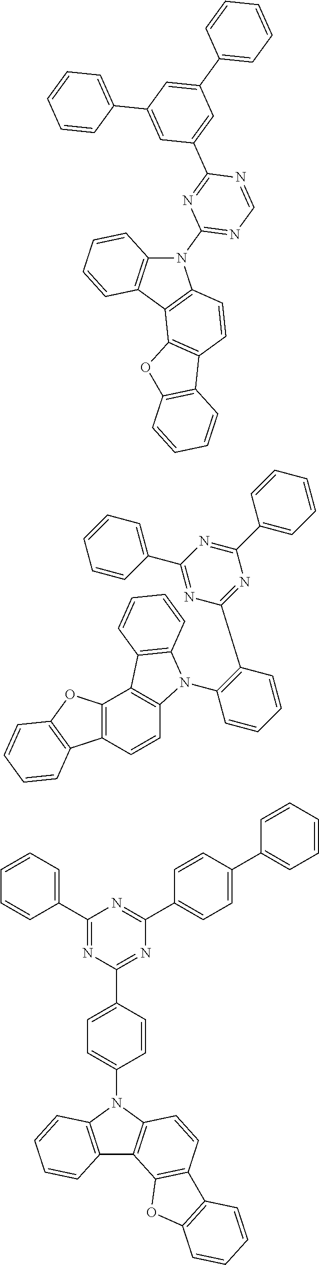 [SMILES: c1ccc(-c2cc(-c3ccccc3)cc(-c3ncnc(-n4c5ccccc5c5c6oc7ccccc7c6ccc54)n3)c2)cc1 ; c1ccc(-c2nc(-c3ccccc3)nc(-c3ccccc3-n3c4ccccc4c4c5oc6ccccc6c5ccc43)n2)cc1 ; c1ccc(-c2ccc(-c3nc(-c4ccccc4)nc(-c4ccc(-n5c6ccccc6c6c7oc8ccccc8c7ccc65)cc4)n3)cc2)cc1]

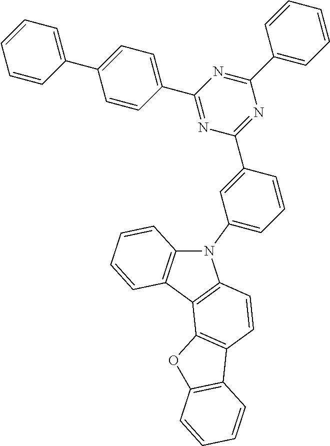 [SMILES: c1ccc(-c2ccc(-c3nc(-c4ccccc4)nc(-c4cccc(-n5c6ccccc6c6c7oc8ccccc8c7ccc65)c4)n3)cc2)cc1]

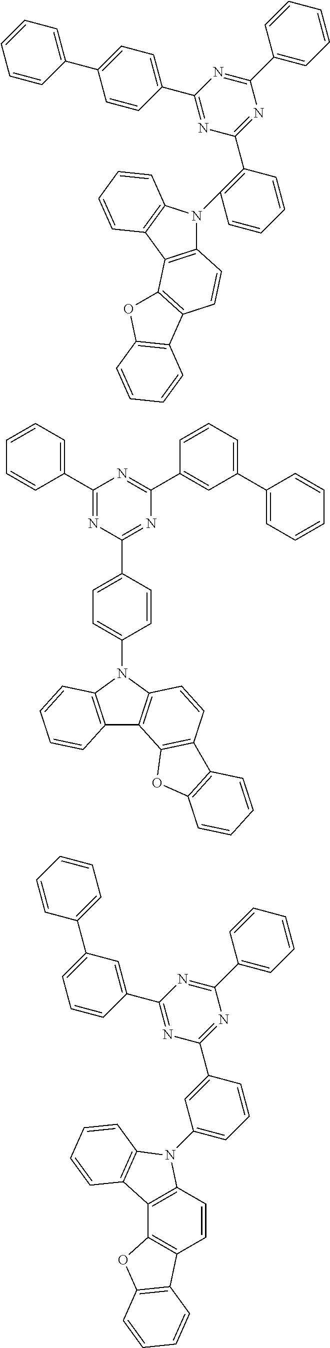

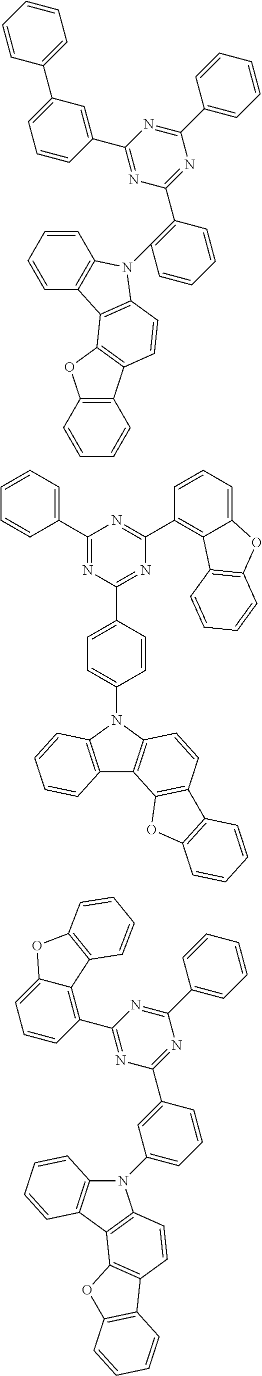

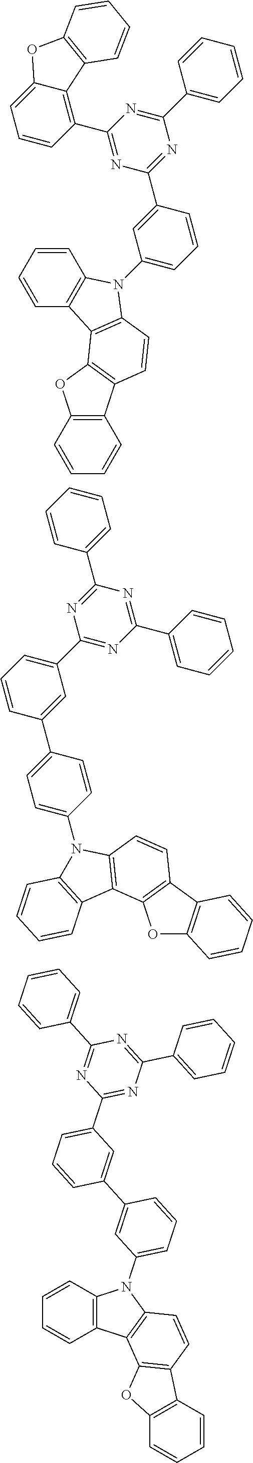

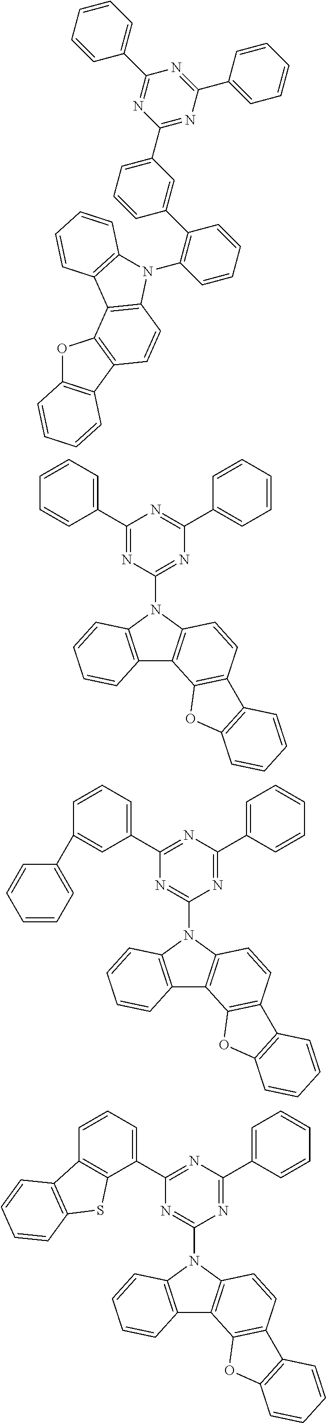

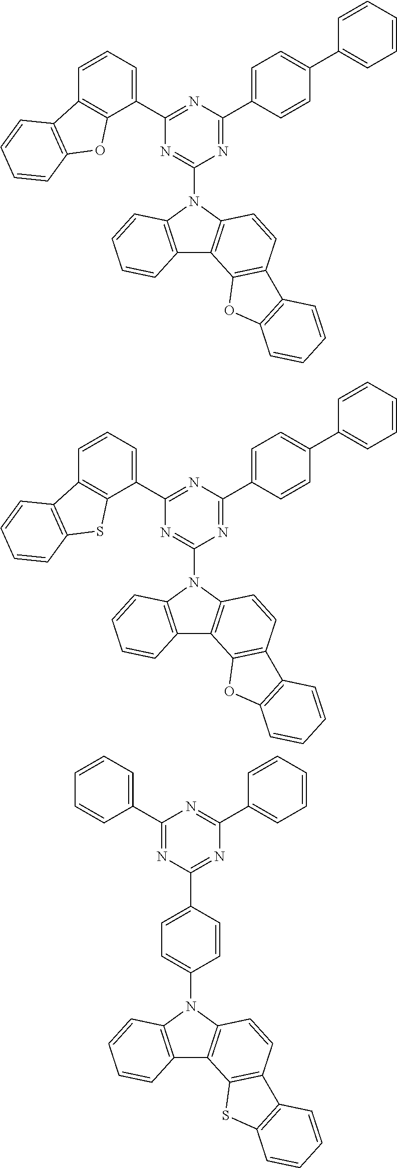

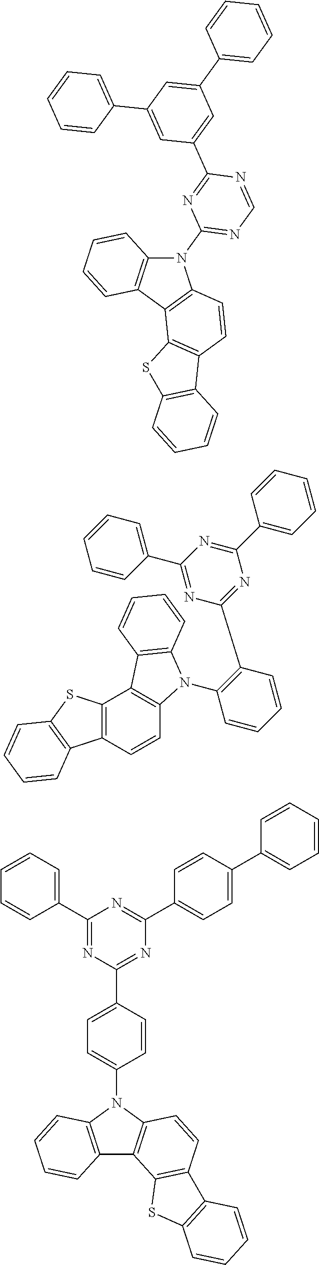 [SMILES: c1ccc(-c2cc(-c3ccccc3)cc(-c3ncnc(-n4c5ccccc5c5c6sc7ccccc7c6ccc54)n3)c2)cc1 ; c1ccc(-c2nc(-c3ccccc3)nc(-c3ccccc3-n3c4ccccc4c4c5sc6ccccc6c5ccc43)n2)cc1 ; c1ccc(-c2ccc(-c3nc(-c4ccccc4)nc(-c4ccc(-n5c6ccccc6c6c7sc8ccccc8c7ccc65)cc4)n3)cc2)cc1]

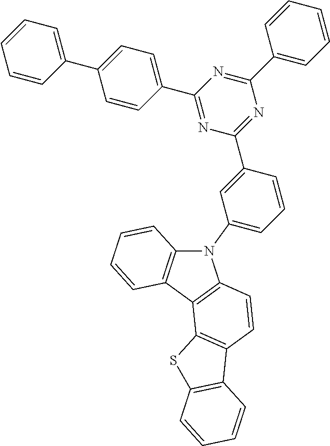 [SMILES: c1ccc(-c2ccc(-c3nc(-c4ccccc4)nc(-c4cccc(-n5c6ccccc6c6c7sc8ccccc8c7ccc65)c4)n3)cc2)cc1]

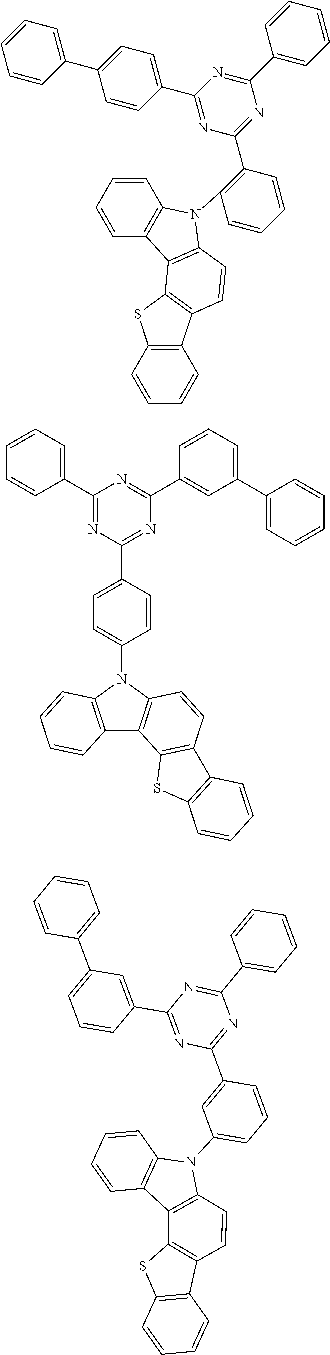

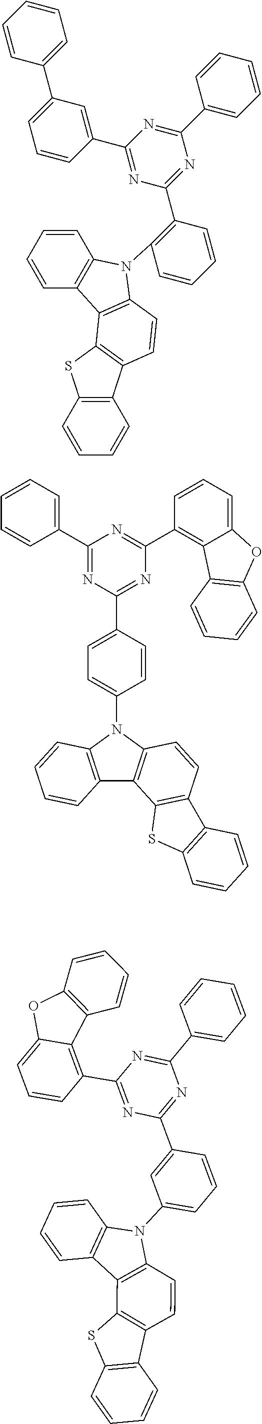

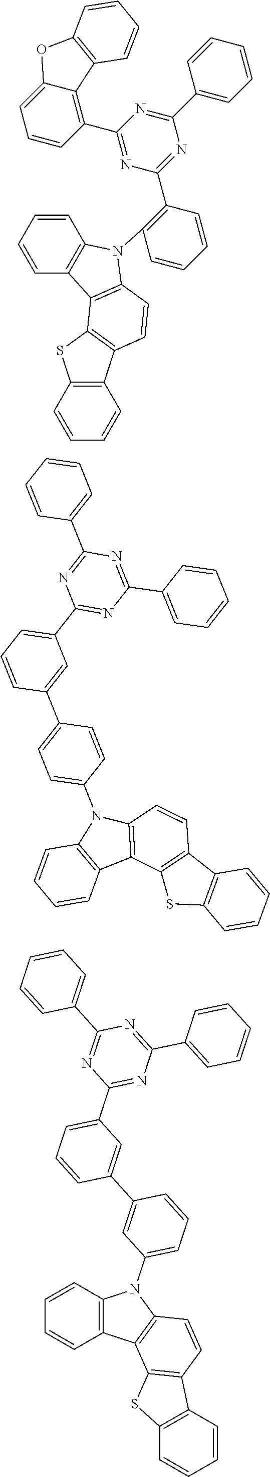

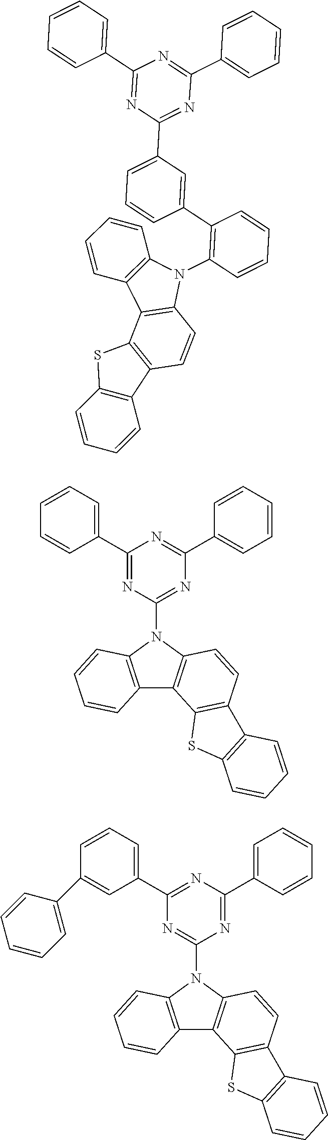

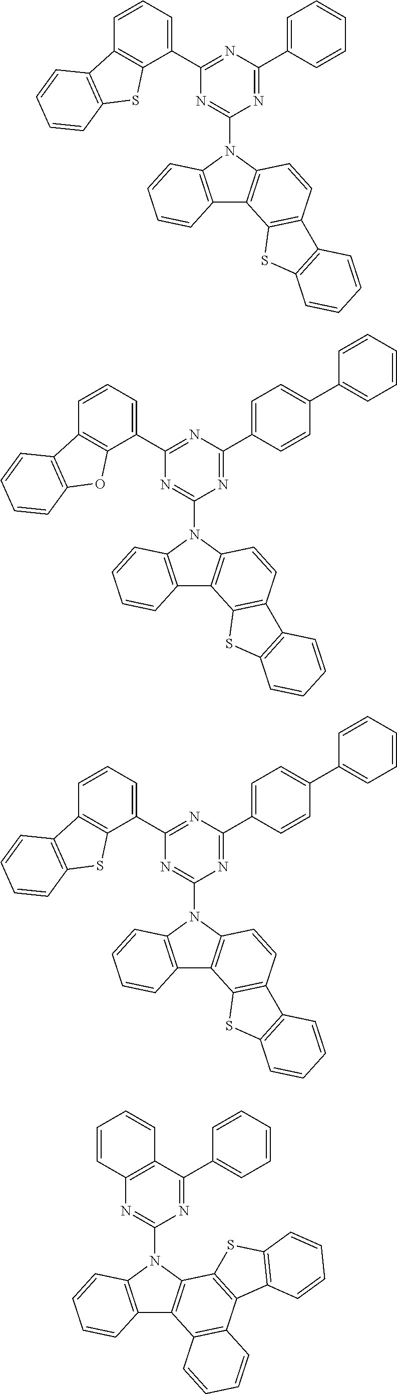

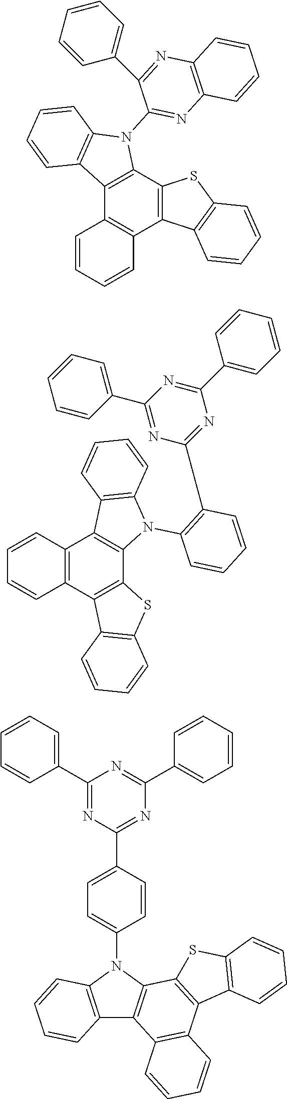

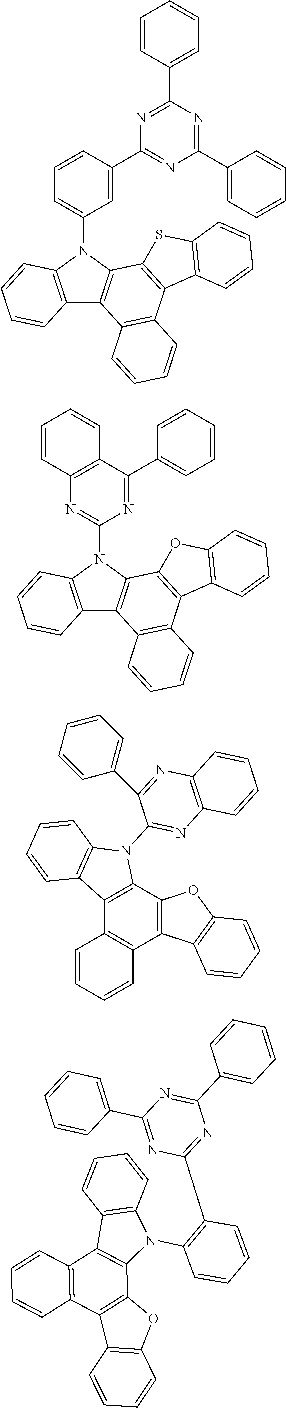

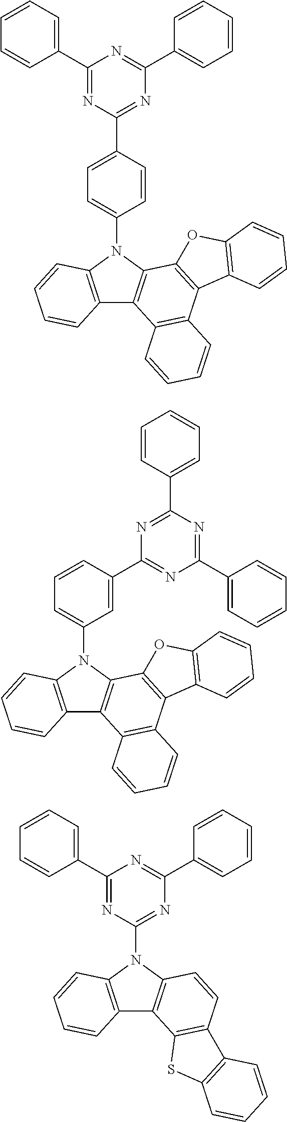

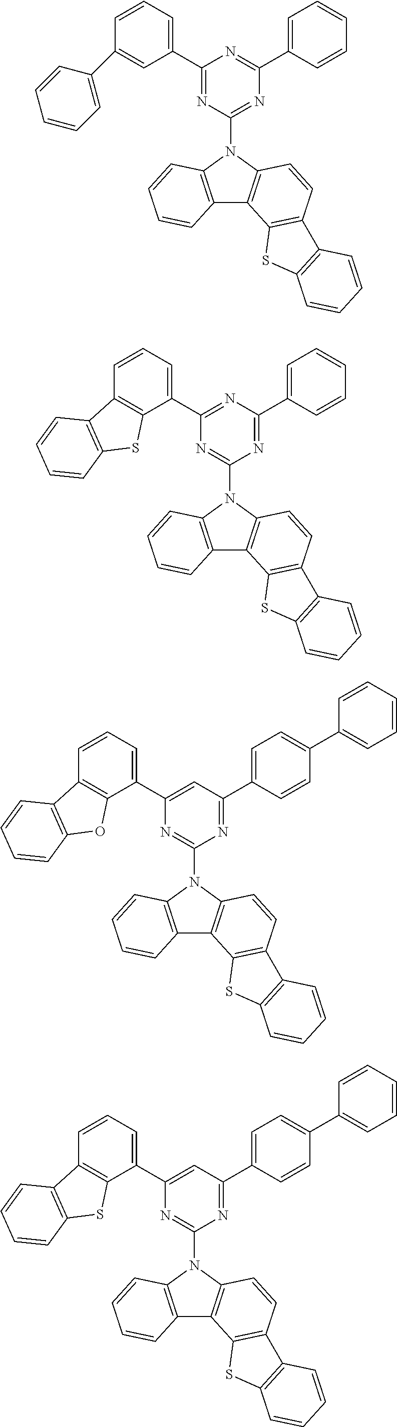

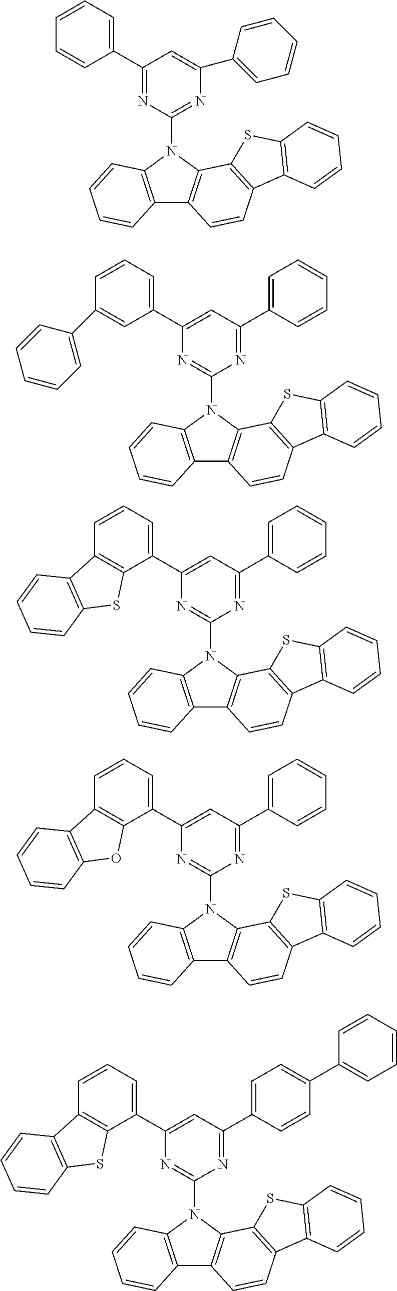

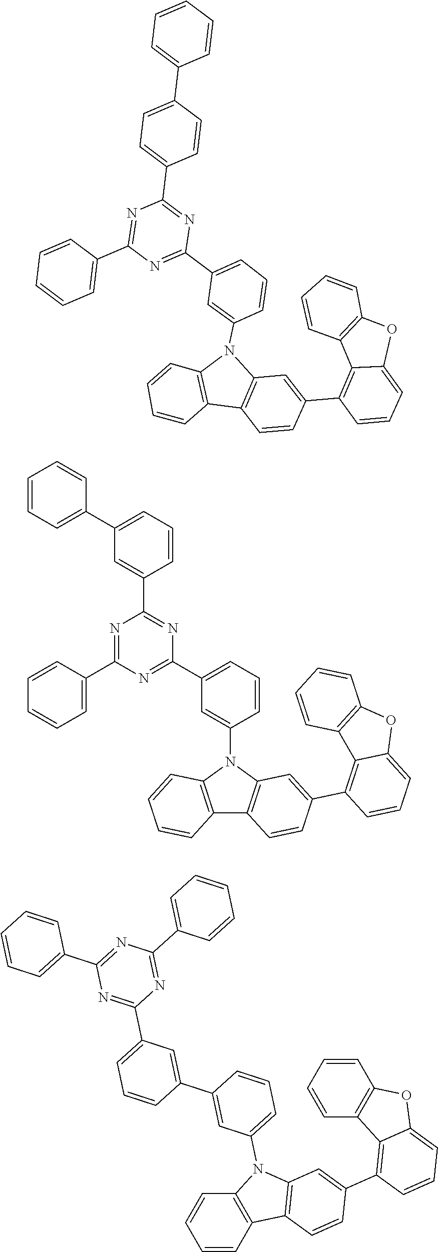

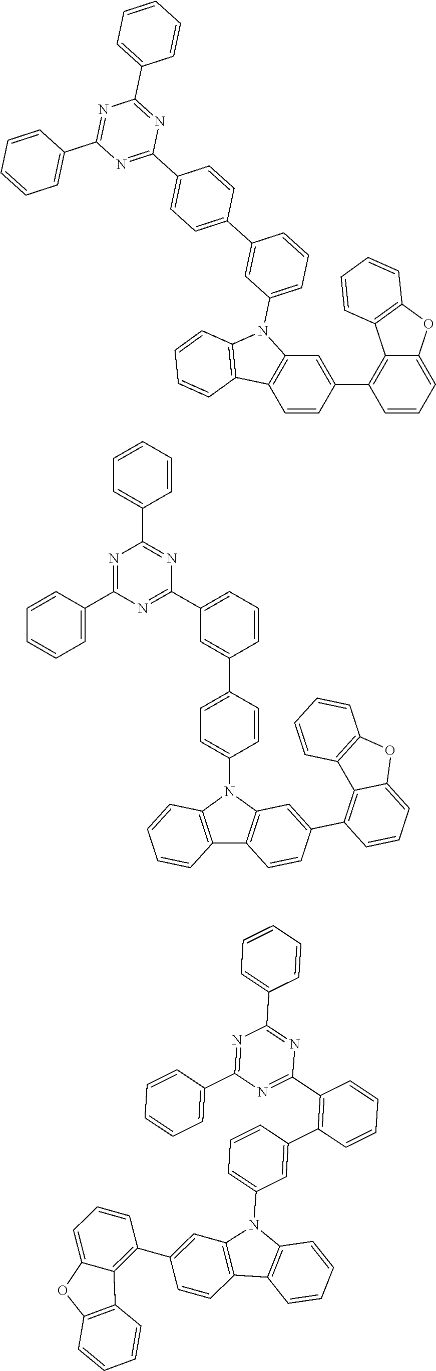 [SMILES: c1ccc(-c2nc(-c3ccccc3)nc(-c3ccc(-c4cccc(-n5c6ccccc6c6ccc(-c7cccc8oc9ccccc9c78)cc65)c4)cc3)n2)cc1 ; c1ccc(-c2nc(-c3ccccc3)nc(-c3cccc(-c4ccc(-n5c6ccccc6c6ccc(-c7cccc8oc9ccccc9c78)cc65)cc4)c3)n2)cc1 ; c1ccc(-c2nc(-c3ccccc3)nc(-c3ccccc3-c3cccc(-n4c5ccccc5c5ccc(-c6cccc7oc8ccccc8c67)cc54)c3)n2)cc1]

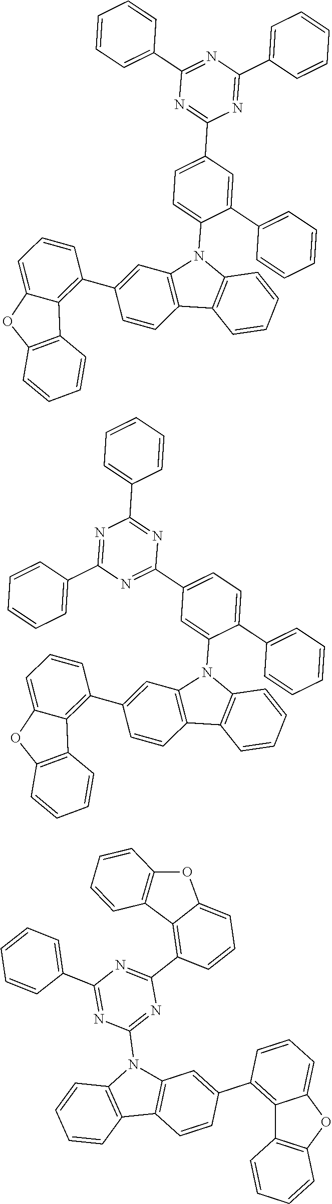 [SMILES: c1ccc(-c2nc(-c3ccccc3)nc(-c3ccc(-n4c5ccccc5c5ccc(-c6cccc7oc8ccccc8c67)cc54)c(-c4ccccc4)c3)n2)cc1 ; c1ccc(-c2nc(-c3ccccc3)nc(-c3ccc(-c4ccccc4)c(-n4c5ccccc5c5ccc(-c6cccc7oc8ccccc8c67)cc54)c3)n2)cc1 ; c1ccc(-c2nc(-c3cccc4oc5ccccc5c34)nc(-n3c4ccccc4c4ccc(-c5cccc6oc7ccccc7c56)cc43)n2)cc1]

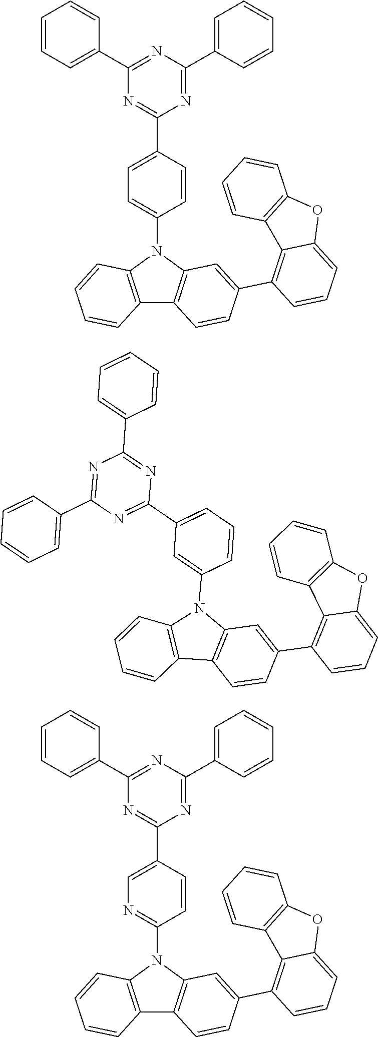

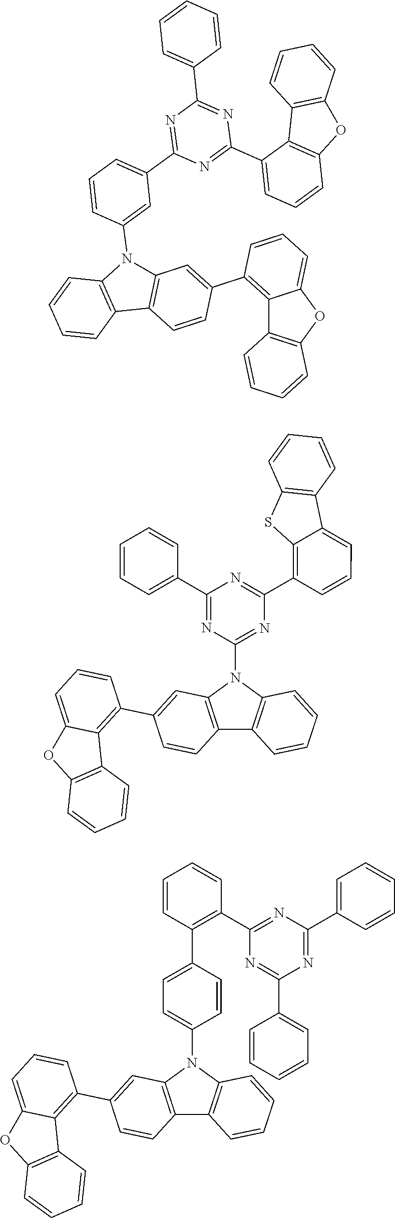

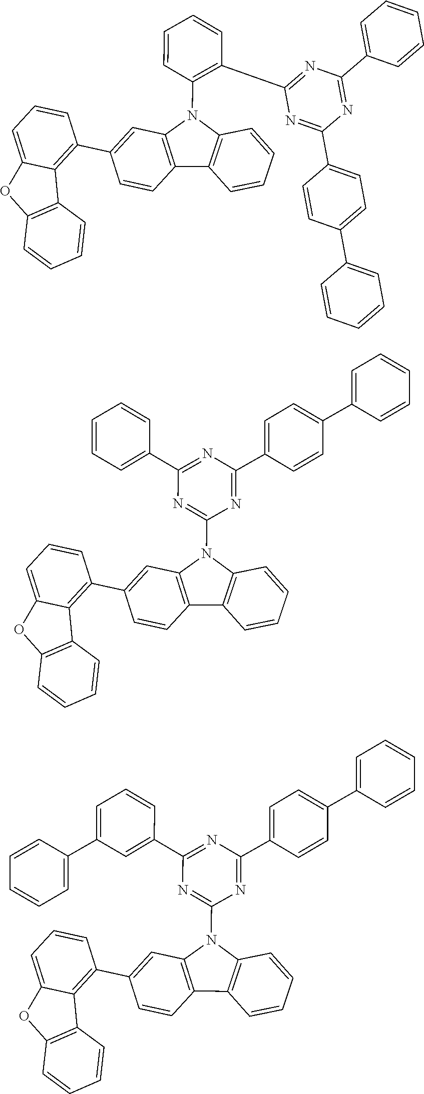

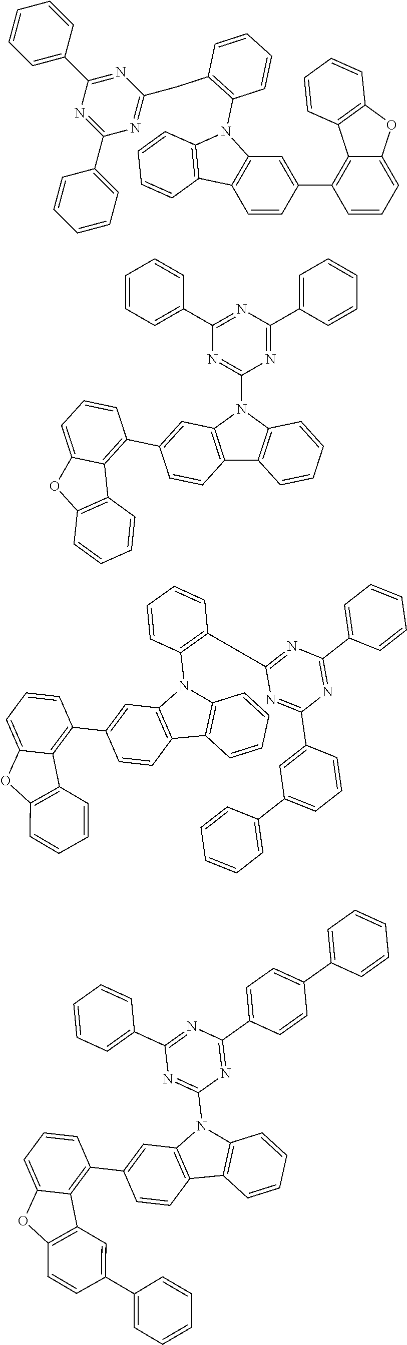 [SMILES: c1ccc(-c2nc(Cc3ccccc3-n3c4ccccc4c4ccc(-c5cccc6oc7ccccc7c56)cc43)nc(-c3ccccc3)n2)cc1 ; c1ccc(-c2nc(-c3ccccc3)nc(-n3c4ccccc4c4ccc(-c5cccc6oc7ccccc7c56)cc43)n2)cc1 ; c1ccc(-c2cccc(-c3nc(Cc4ccccc4-n4c5ccccc5c5ccc(-c6cccc7oc8ccccc8c67)cc54)nc(-c4ccccc4)n3)c2)cc1 ; c1ccc(-c2ccc(-c3nc(-c4ccccc4)nc(-n4c5ccccc5c5ccc(-c6cccc7oc8ccc(-c9ccccc9)cc8c67)cc54)n3)cc2)cc1]

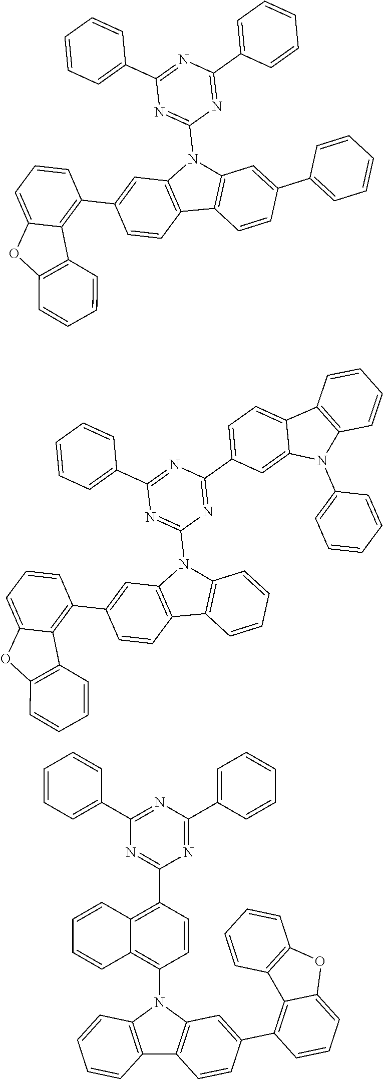

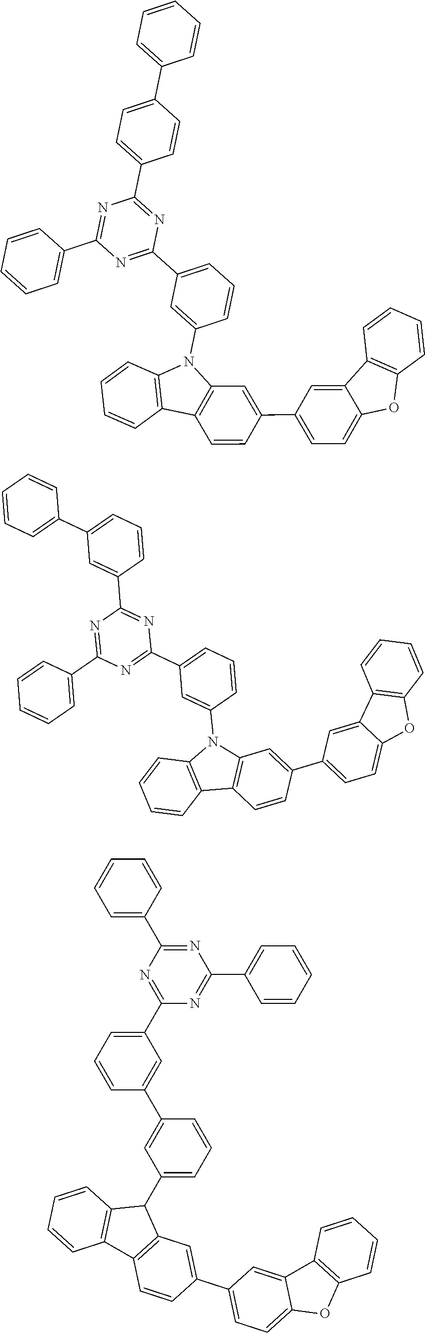 [SMILES: c1ccc(-c2ccc(-c3nc(-c4ccccc4)nc(-c4cccc(-n5c6ccccc6c6ccc(-c7ccc8oc9ccccc9c8c7)cc65)c4)n3)cc2)cc1 ; c1ccc(-c2cccc(-c3nc(-c4ccccc4)nc(-c4cccc(-n5c6ccccc6c6ccc(-c7ccc8oc9ccccc9c8c7)cc65)c4)n3)c2)cc1 ; c1ccc(-c2nc(-c3ccccc3)nc(-c3cccc(-c4cccc(C5c6ccccc6-c6ccc(-c7ccc8oc9ccccc9c8c7)cc65)c4)c3)n2)cc1]

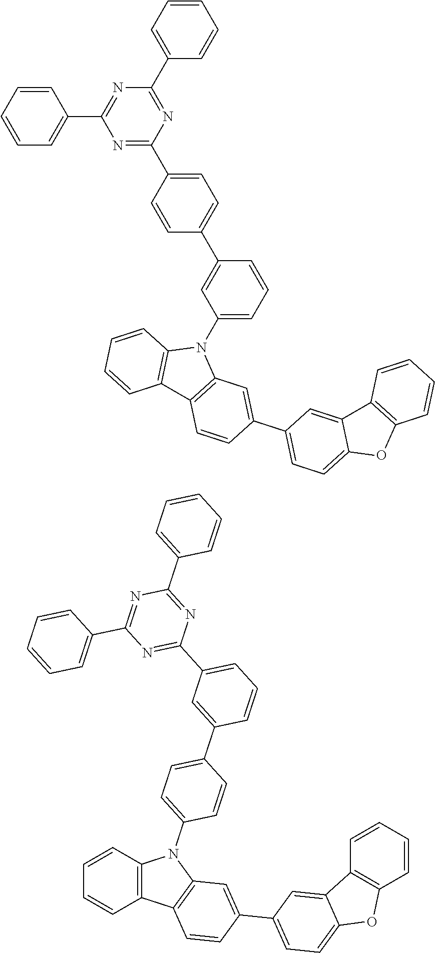 [SMILES: c1ccc(-c2nc(-c3ccccc3)nc(-c3ccc(-c4cccc(-n5c6ccccc6c6ccc(-c7ccc8oc9ccccc9c8c7)cc65)c4)cc3)n2)cc1 ; c1ccc(-c2nc(-c3ccccc3)nc(-c3cccc(-c4ccc(-n5c6ccccc6c6ccc(-c7ccc8oc9ccccc9c8c7)cc65)cc4)c3)n2)cc1]

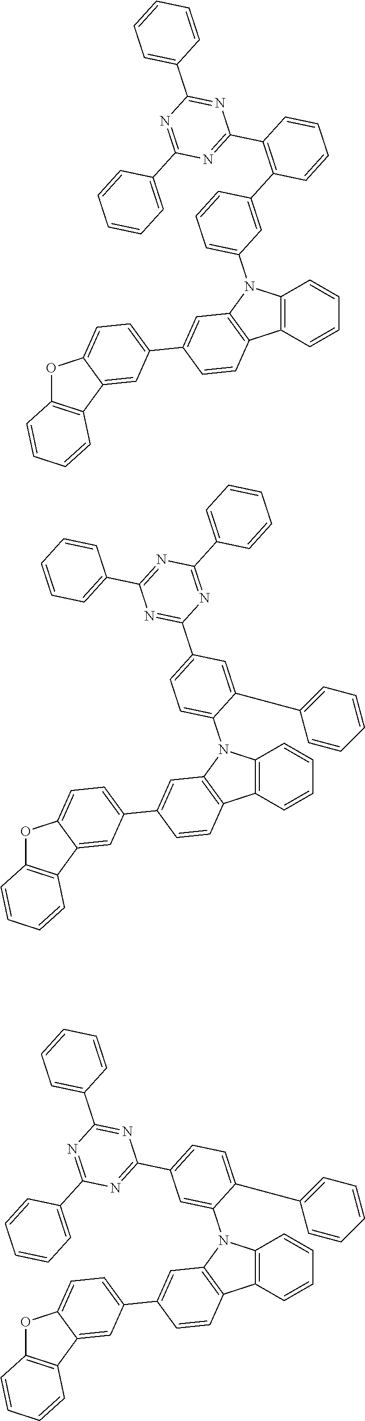 [SMILES: c1ccc(-c2nc(-c3ccccc3)nc(-c3ccccc3-c3cccc(-n4c5ccccc5c5ccc(-c6ccc7oc8ccccc8c7c6)cc54)c3)n2)cc1 ; c1ccc(Cc2cc(-c3nc(-c4ccccc4)nc(-c4ccccc4)n3)ccc2-n2c3ccccc3c3ccc(-c4ccc5oc6ccccc6c5c4)cc32)cc1 ; c1ccc(Cc2ccc(-c3nc(-c4ccccc4)nc(-c4ccccc4)n3)cc2-n2c3ccccc3c3ccc(-c4ccc5oc6ccccc6c5c4)cc32)cc1]

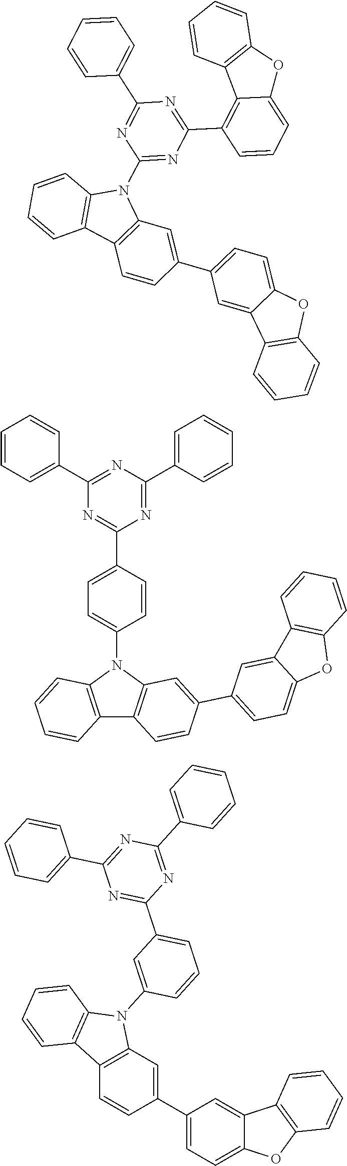

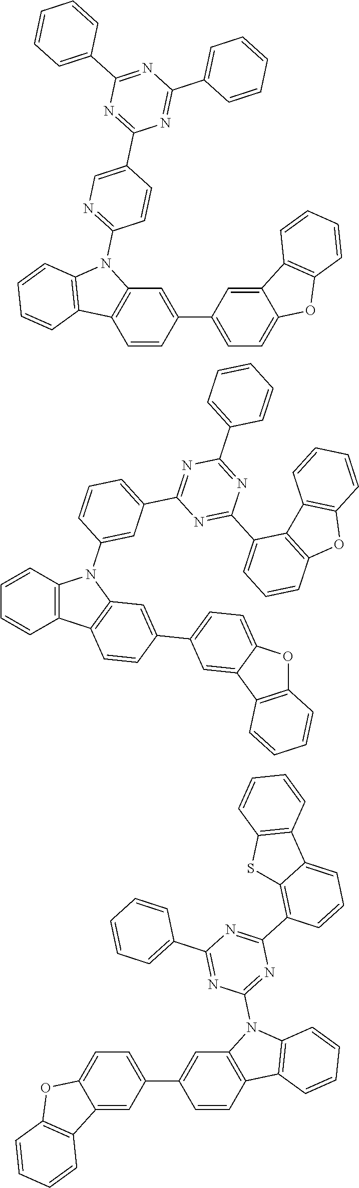

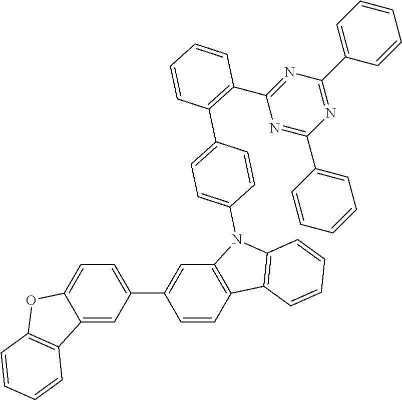 [SMILES: c1ccc(-c2nc(-c3ccccc3)nc(-c3ccccc3-c3ccc(-n4c5ccccc5c5ccc(-c6ccc7oc8ccccc8c7c6)cc54)cc3)n2)cc1]

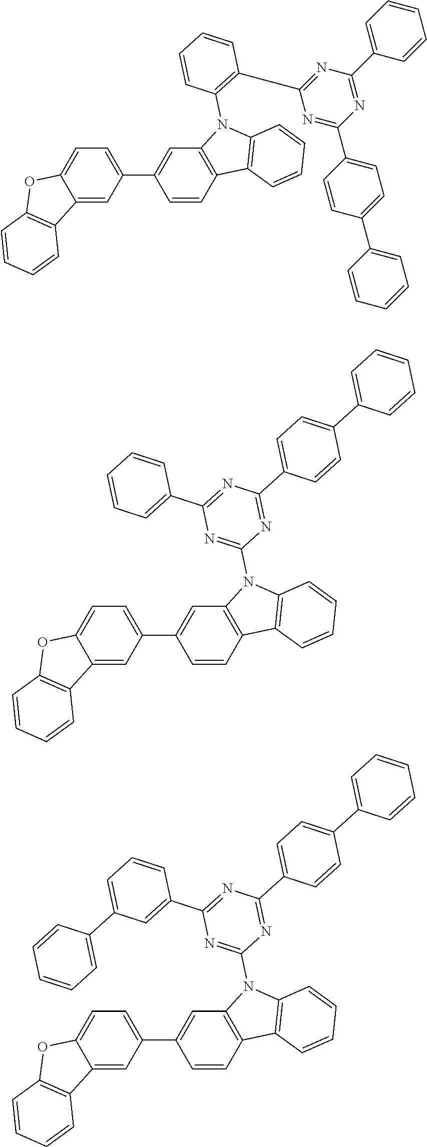

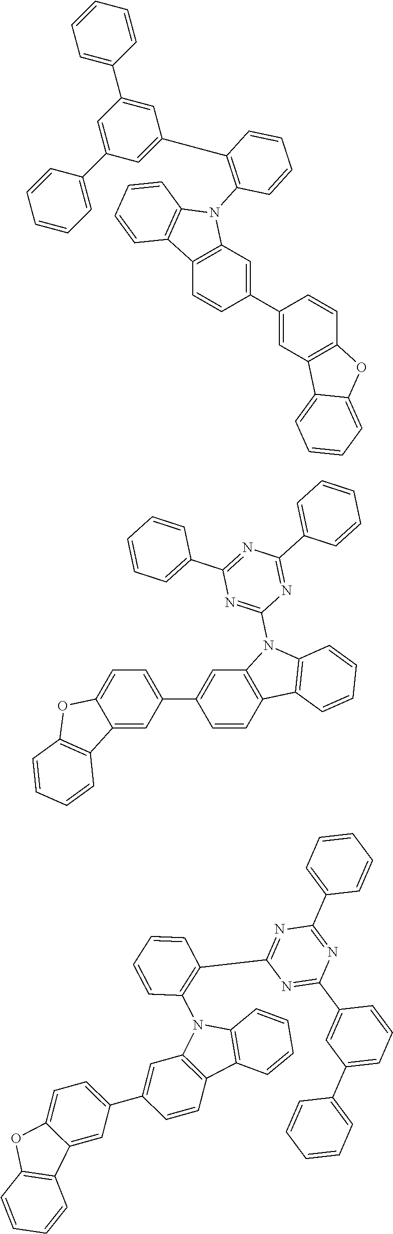

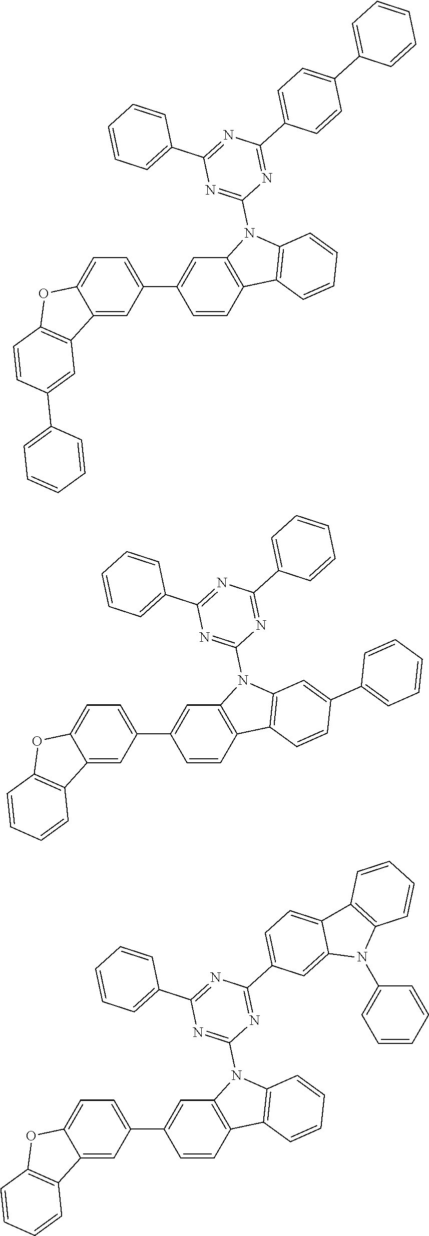 [SMILES: c1ccc(-c2ccc(-c3nc(-c4ccccc4)nc(-n4c5ccccc5c5ccc(-c6ccc7oc8ccc(-c9ccccc9)cc8c7c6)cc54)n3)cc2)cc1 ; c1ccc(-c2ccc3c4ccc(-c5ccc6oc7ccccc7c6c5)cc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4ccc(-c5ccc6oc7ccccc7c6c5)cc43)n2)cc1]

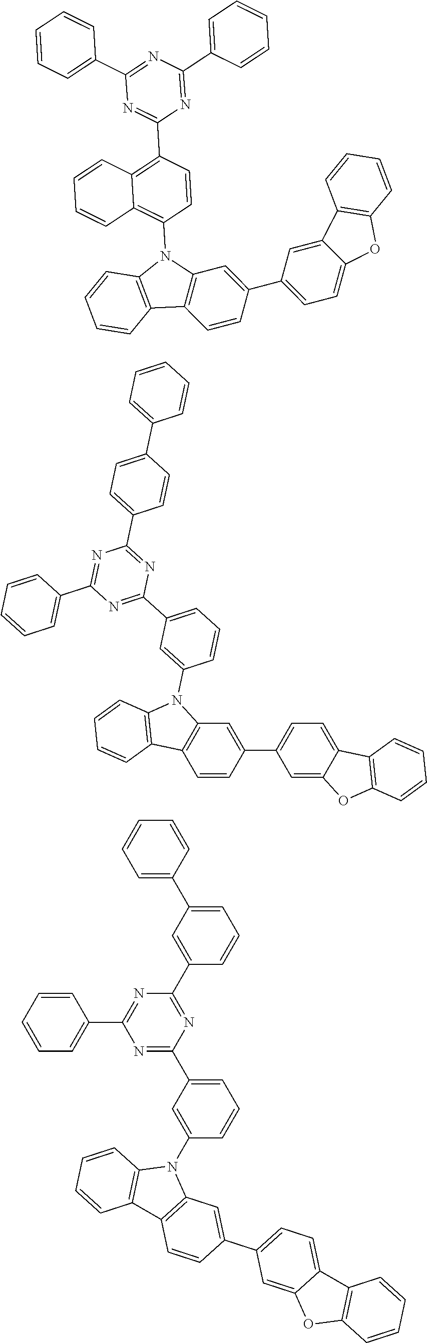

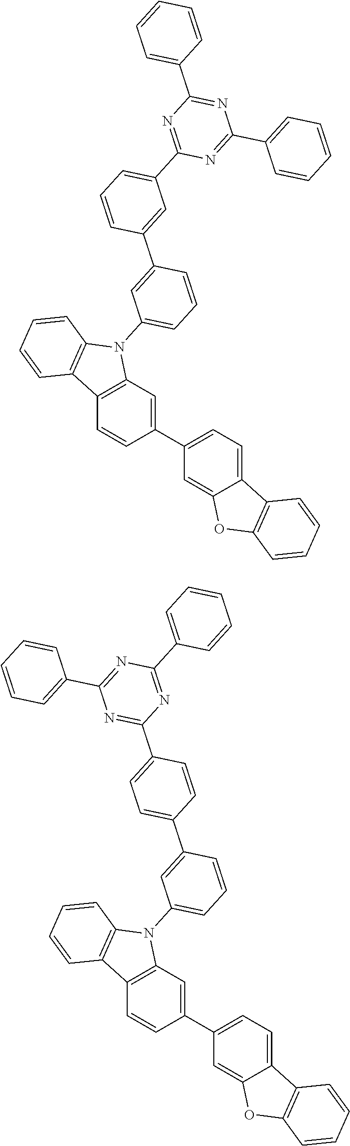 [SMILES: c1ccc(-c2nc(-c3ccccc3)nc(-c3cccc(-c4cccc(-n5c6ccccc6c6ccc(-c7ccc8c(c7)oc7ccccc78)cc65)c4)c3)n2)cc1 ; c1ccc(-c2nc(-c3ccccc3)nc(-c3ccc(-c4cccc(-n5c6ccccc6c6ccc(-c7ccc8c(c7)oc7ccccc78)cc65)c4)cc3)n2)cc1]

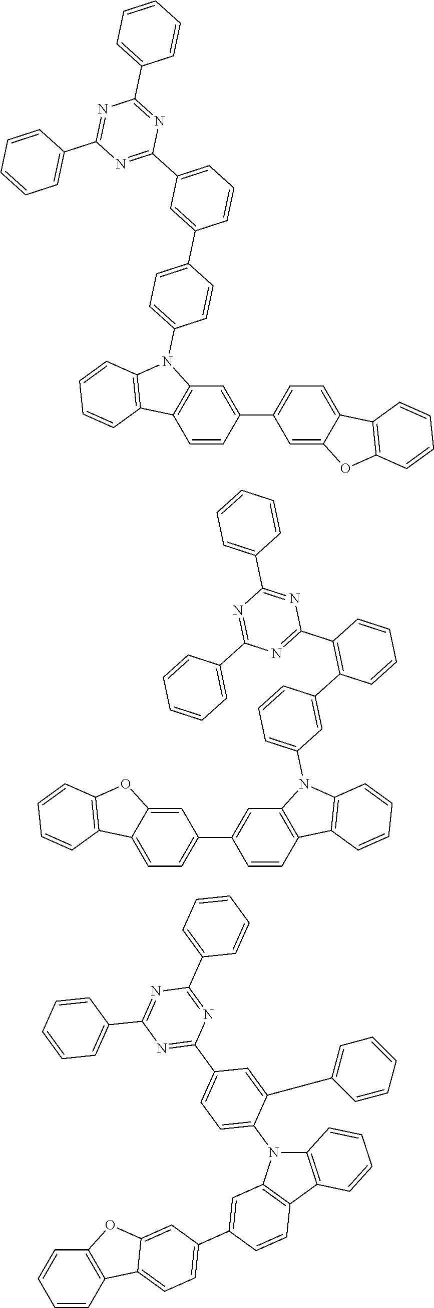

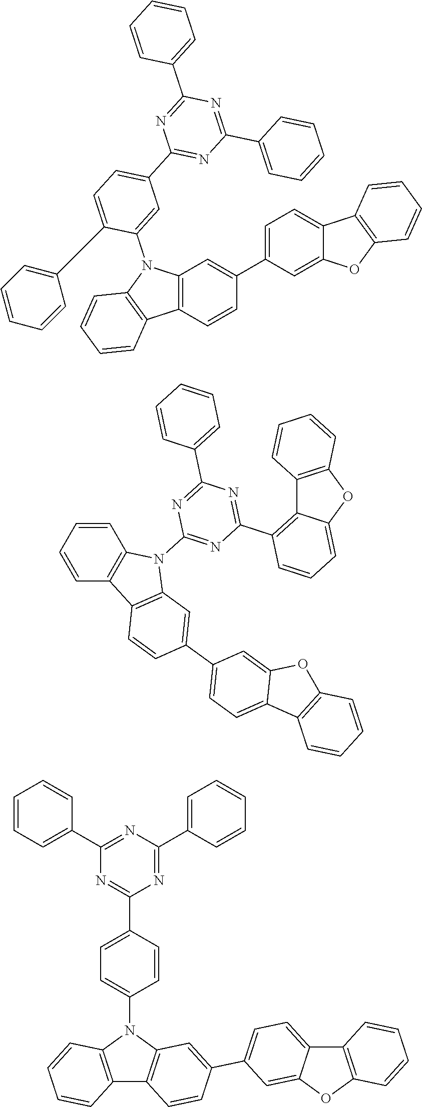 [SMILES: c1ccc(Cc2ccc(-c3nc(-c4ccccc4)nc(-c4ccccc4)n3)cc2-n2c3ccccc3c3ccc(-c4ccc5c(c4)oc4ccccc45)cc32)cc1 ; c1ccc(-c2nc(-c3cccc4oc5ccccc5c34)nc(-n3c4ccccc4c4ccc(-c5ccc6c(c5)oc5ccccc56)cc43)n2)cc1 ; c1ccc(-c2nc(-c3ccccc3)nc(-c3ccc(-n4c5ccccc5c5ccc(-c6ccc7c(c6)oc6ccccc67)cc54)cc3)n2)cc1]

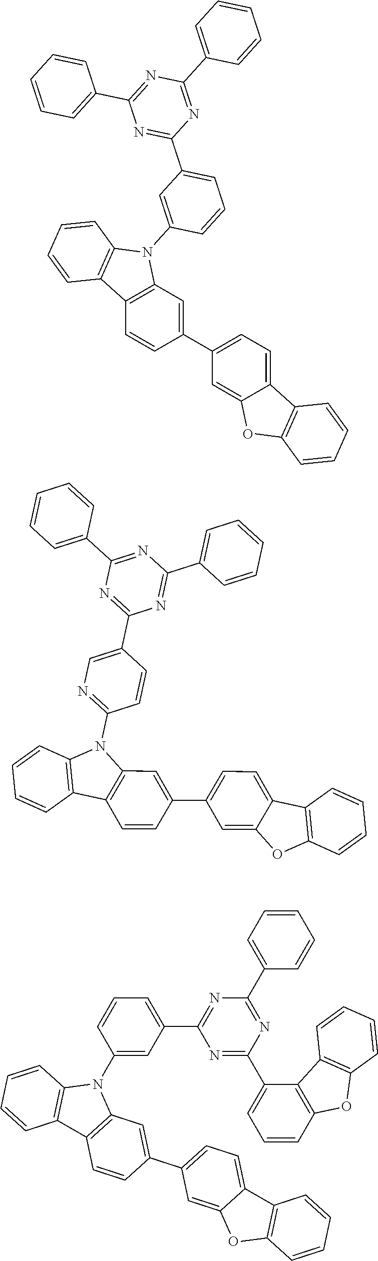 [SMILES: c1ccc(-c2nc(-c3ccccc3)nc(-c3cccc(-n4c5ccccc5c5ccc(-c6ccc7c(c6)oc6ccccc67)cc54)c3)n2)cc1 ; c1ccc(-c2nc(-c3ccccc3)nc(-c3ccc(-n4c5ccccc5c5ccc(-c6ccc7c(c6)oc6ccccc67)cc54)nc3)n2)cc1 ; c1ccc(-c2nc(-c3cccc(-n4c5ccccc5c5ccc(-c6ccc7c(c6)oc6ccccc67)cc54)c3)nc(-c3cccc4oc5ccccc5c34)n2)cc1]

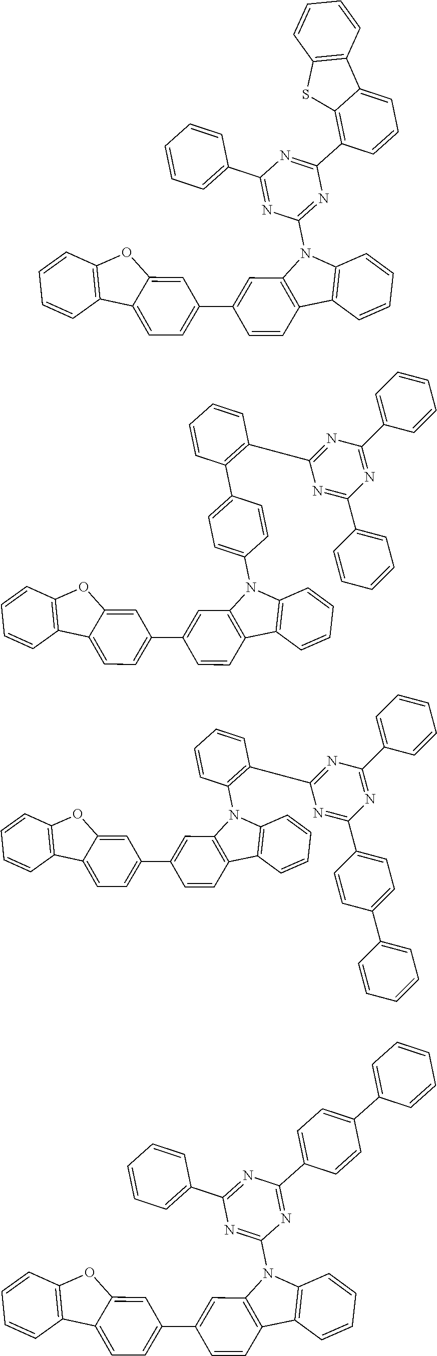

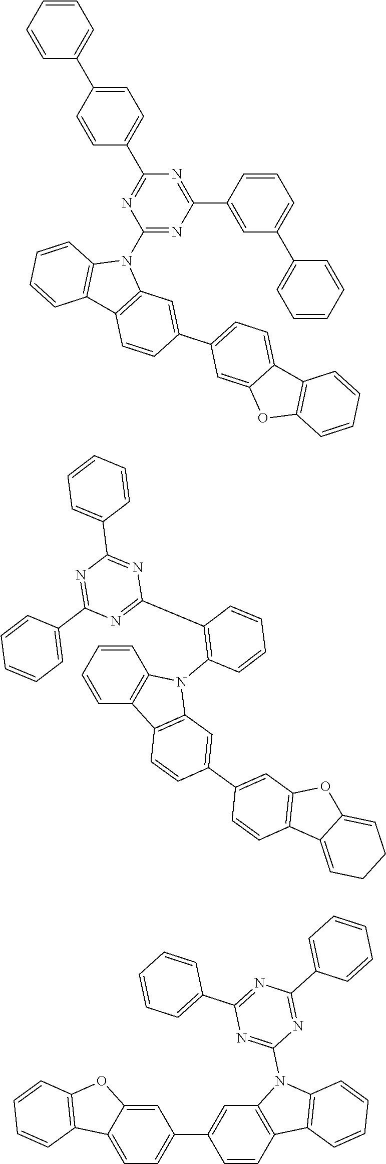

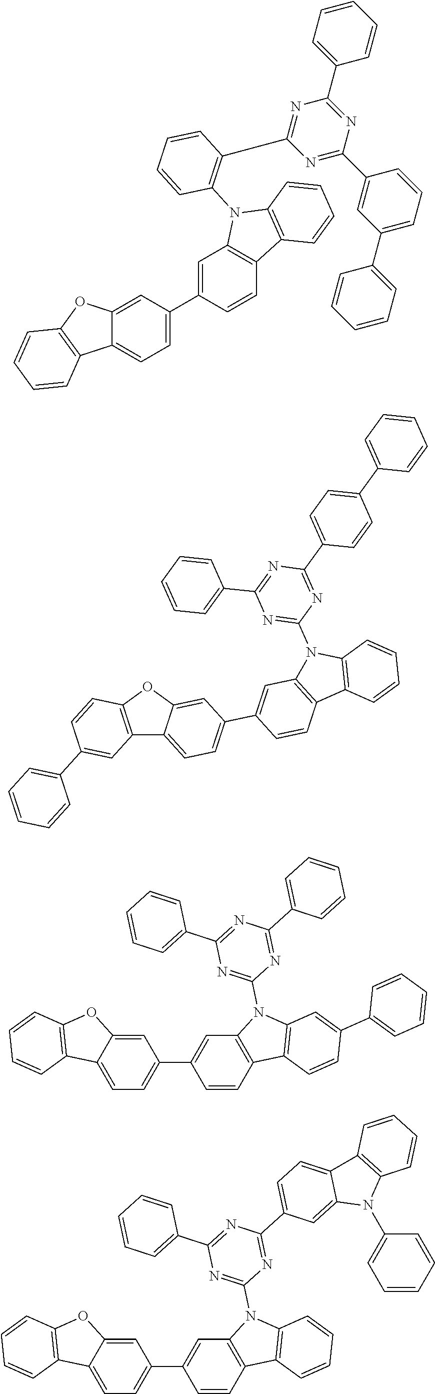 [SMILES: c1ccc(-c2cccc(-c3nc(Cc4ccccc4-n4c5ccccc5c5ccc(-c6ccc7c(c6)oc6ccccc67)cc54)nc(-c4ccccc4)n3)c2)cc1 ; c1ccc(-c2ccc(-c3nc(-c4ccccc4)nc(-n4c5ccccc5c5ccc(-c6ccc7c(c6)oc6ccc(-c8ccccc8)cc67)cc54)n3)cc2)cc1 ; c1ccc(-c2ccc3c4ccc(-c5ccc6c(c5)oc5ccccc56)cc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4ccc(-c5ccc6c(c5)oc5ccccc56)cc43)n2)cc1]

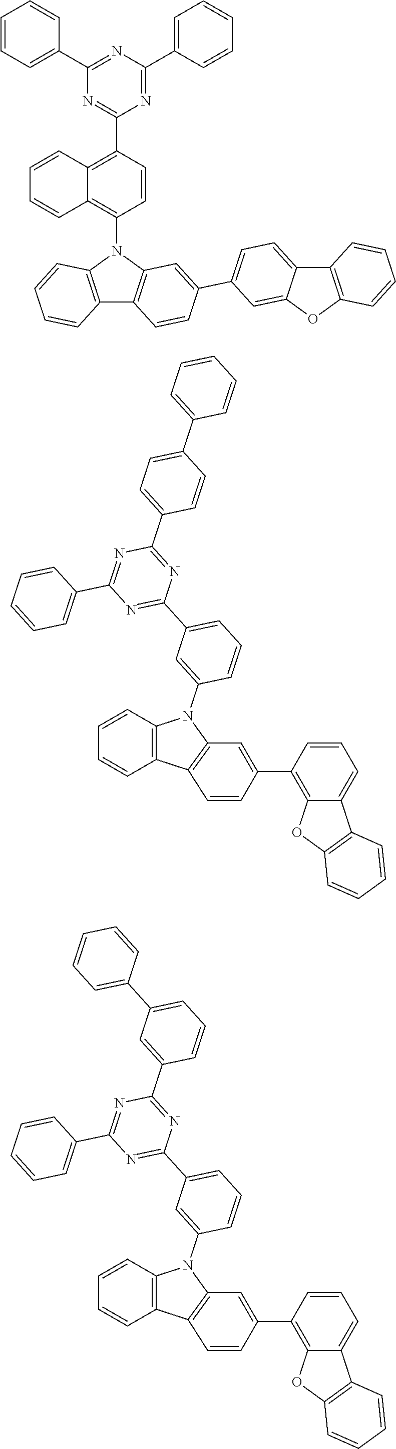

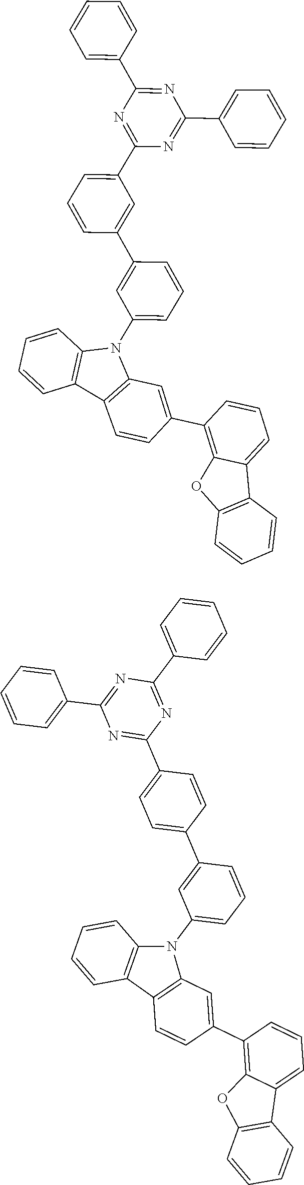 [SMILES: c1ccc(-c2nc(-c3ccccc3)nc(-c3cccc(-c4cccc(-n5c6ccccc6c6ccc(-c7cccc8c7oc7ccccc78)cc65)c4)c3)n2)cc1 ; c1ccc(-c2nc(-c3ccccc3)nc(-c3ccc(-c4cccc(-n5c6ccccc6c6ccc(-c7cccc8c7oc7ccccc78)cc65)c4)cc3)n2)cc1]

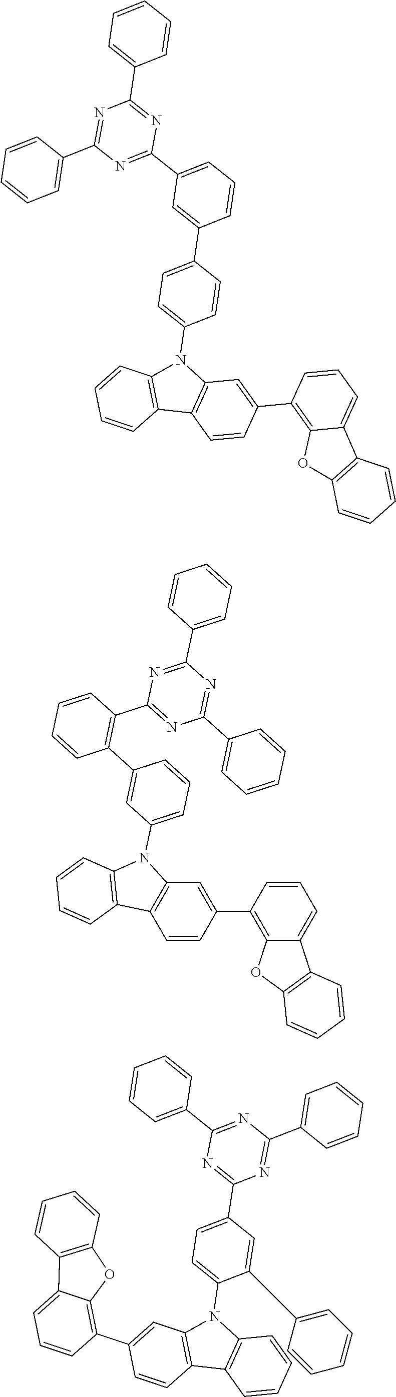 [SMILES: c1ccc(-c2nc(-c3ccccc3)nc(-c3cccc(-c4ccc(-n5c6ccccc6c6ccc(-c7cccc8c7oc7ccccc78)cc65)cc4)c3)n2)cc1 ; c1ccc(-c2nc(-c3ccccc3)nc(-c3ccccc3-c3cccc(-n4c5ccccc5c5ccc(-c6cccc7c6oc6ccccc67)cc54)c3)n2)cc1 ; c1ccc(Cc2cc(-c3nc(-c4ccccc4)nc(-c4ccccc4)n3)ccc2-n2c3ccccc3c3ccc(-c4cccc5c4oc4ccccc45)cc32)cc1]

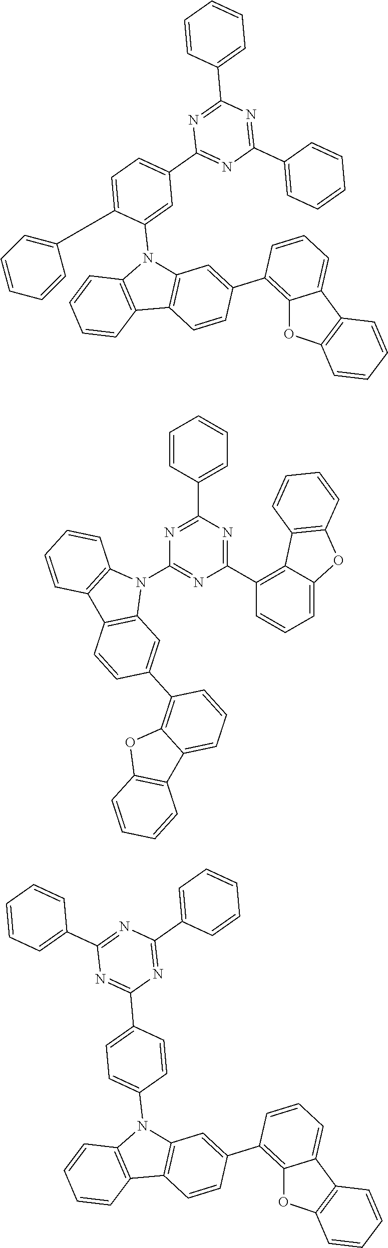 [SMILES: c1ccc(Cc2ccc(-c3nc(-c4ccccc4)nc(-c4ccccc4)n3)cc2-n2c3ccccc3c3ccc(-c4cccc5c4oc4ccccc45)cc32)cc1 ; c1ccc(-c2nc(-c3cccc4oc5ccccc5c34)nc(-n3c4ccccc4c4ccc(-c5cccc6c5oc5ccccc56)cc43)n2)cc1 ; c1ccc(-c2nc(-c3ccccc3)nc(-c3ccc(-n4c5ccccc5c5ccc(-c6cccc7c6oc6ccccc67)cc54)cc3)n2)cc1]

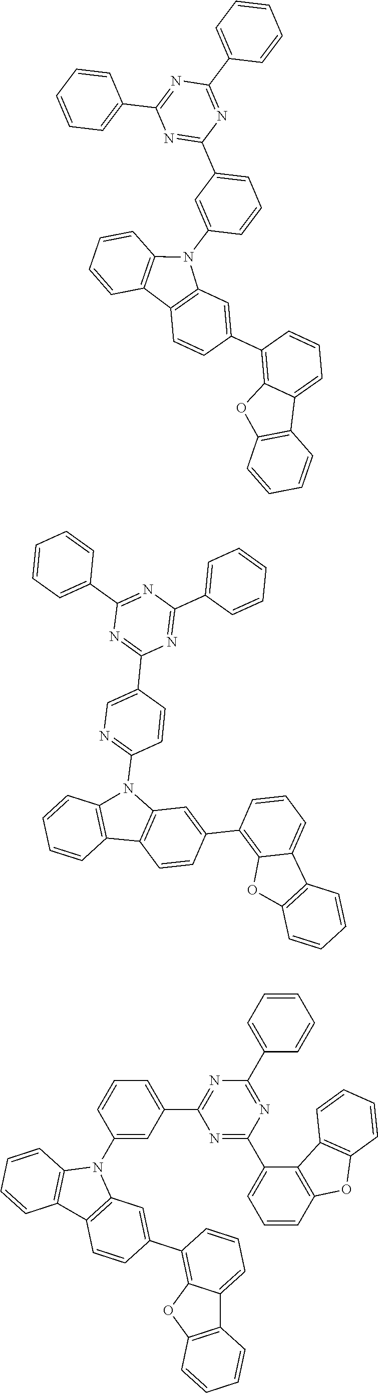 [SMILES: c1ccc(-c2nc(-c3ccccc3)nc(-c3cccc(-n4c5ccccc5c5ccc(-c6cccc7c6oc6ccccc67)cc54)c3)n2)cc1 ; c1ccc(-c2nc(-c3ccccc3)nc(-c3ccc(-n4c5ccccc5c5ccc(-c6cccc7c6oc6ccccc67)cc54)nc3)n2)cc1 ; c1ccc(-c2nc(-c3cccc(-n4c5ccccc5c5ccc(-c6cccc7c6oc6ccccc67)cc54)c3)nc(-c3cccc4oc5ccccc5c34)n2)cc1]

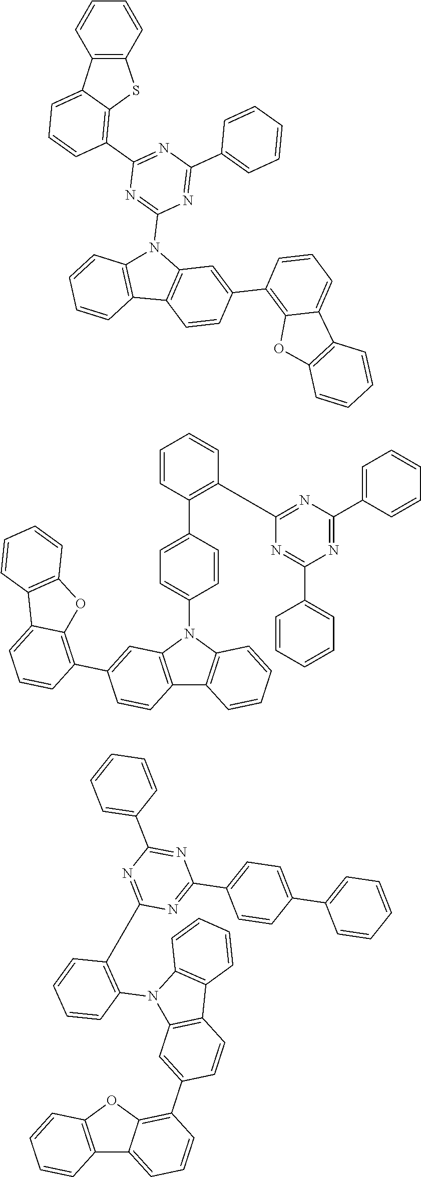 [SMILES: c1ccc(-c2nc(-c3cccc4c3sc3ccccc34)nc(-n3c4ccccc4c4ccc(-c5cccc6c5oc5ccccc56)cc43)n2)cc1 ; c1ccc(-c2nc(Cc3ccccc3-c3ccc(-n4c5ccccc5c5ccc(-c6cccc7c6oc6ccccc67)cc54)cc3)nc(-c3ccccc3)n2)cc1 ; c1ccc(-c2ccc(-c3nc(Cc4ccccc4-n4c5ccccc5c5ccc(-c6cccc7c6oc6ccccc67)cc54)nc(-c4ccccc4)n3)cc2)cc1]

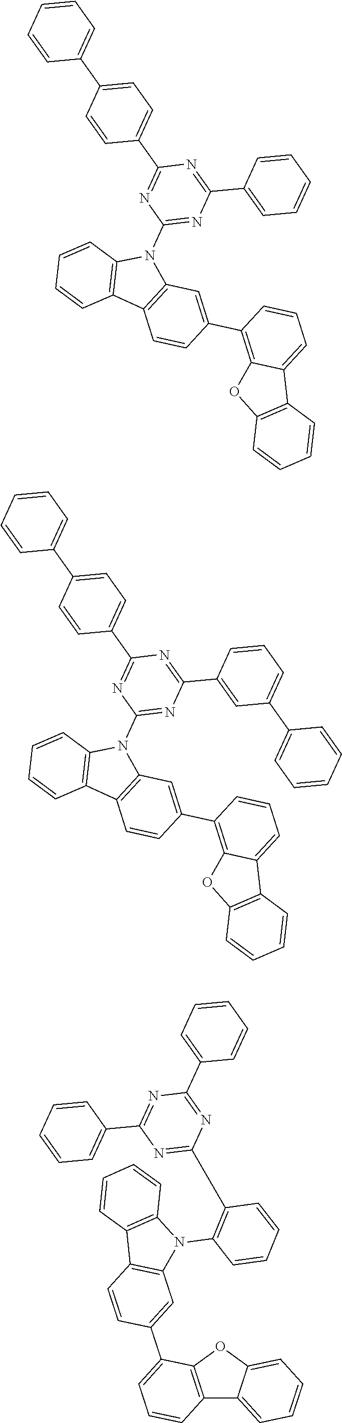 [SMILES: c1ccc(-c2ccc(-c3nc(-c4ccccc4)nc(-n4c5ccccc5c5ccc(-c6cccc7c6oc6ccccc67)cc54)n3)cc2)cc1 ; c1ccc(-c2ccc(-c3nc(-c4cccc(-c5ccccc5)c4)nc(-n4c5ccccc5c5ccc(-c6cccc7c6oc6ccccc67)cc54)n3)cc2)cc1 ; c1ccc(-c2nc(Cc3ccccc3-n3c4ccccc4c4ccc(-c5cccc6c5oc5ccccc56)cc43)nc(-c3ccccc3)n2)cc1]

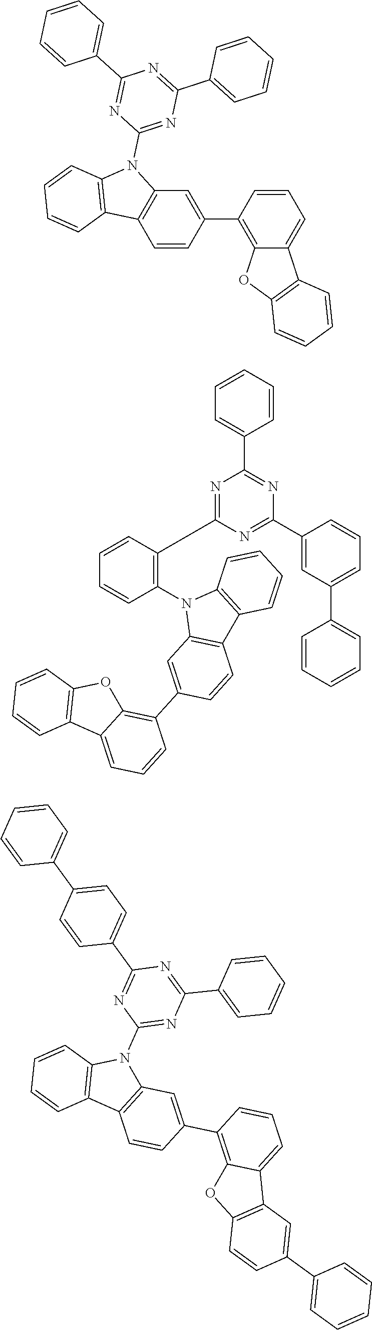 [SMILES: c1ccc(-c2nc(-c3ccccc3)nc(-n3c4ccccc4c4ccc(-c5cccc6c5oc5ccccc56)cc43)n2)cc1 ; c1ccc(-c2cccc(-c3nc(Cc4ccccc4-n4c5ccccc5c5ccc(-c6cccc7c6oc6ccccc67)cc54)nc(-c4ccccc4)n3)c2)cc1 ; c1ccc(-c2ccc(-c3nc(-c4ccccc4)nc(-n4c5ccccc5c5ccc(-c6cccc7c6oc6ccc(-c8ccccc8)cc67)cc54)n3)cc2)cc1]

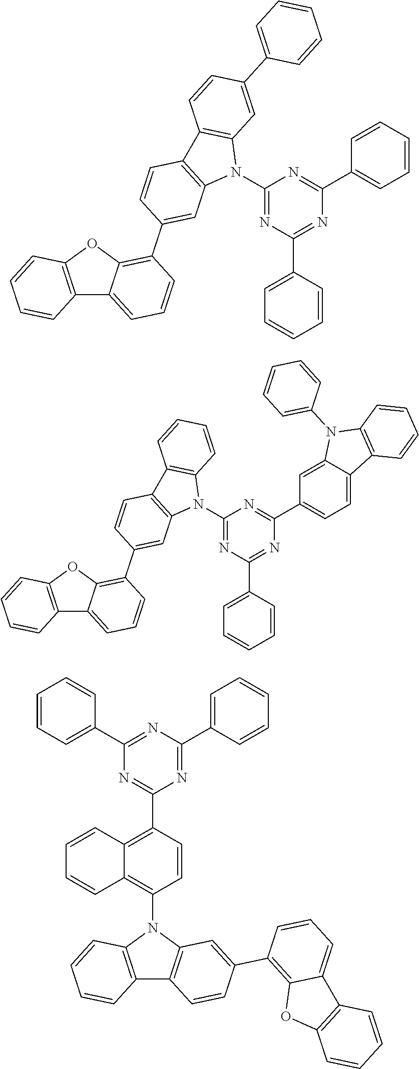

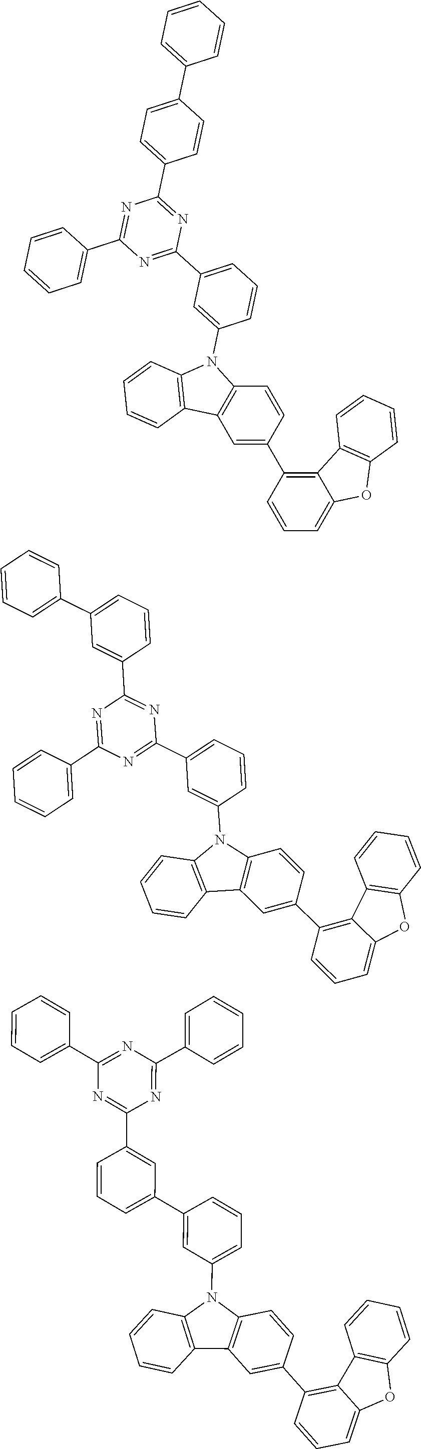 [SMILES: c1ccc(-c2ccc(-c3nc(-c4ccccc4)nc(-c4cccc(-n5c6ccccc6c6cc(-c7cccc8oc9ccccc9c78)ccc65)c4)n3)cc2)cc1 ; c1ccc(-c2cccc(-c3nc(-c4ccccc4)nc(-c4cccc(-n5c6ccccc6c6cc(-c7cccc8oc9ccccc9c78)ccc65)c4)n3)c2)cc1 ; c1ccc(-c2nc(-c3ccccc3)nc(-c3cccc(-c4cccc(-n5c6ccccc6c6cc(-c7cccc8oc9ccccc9c78)ccc65)c4)c3)n2)cc1]

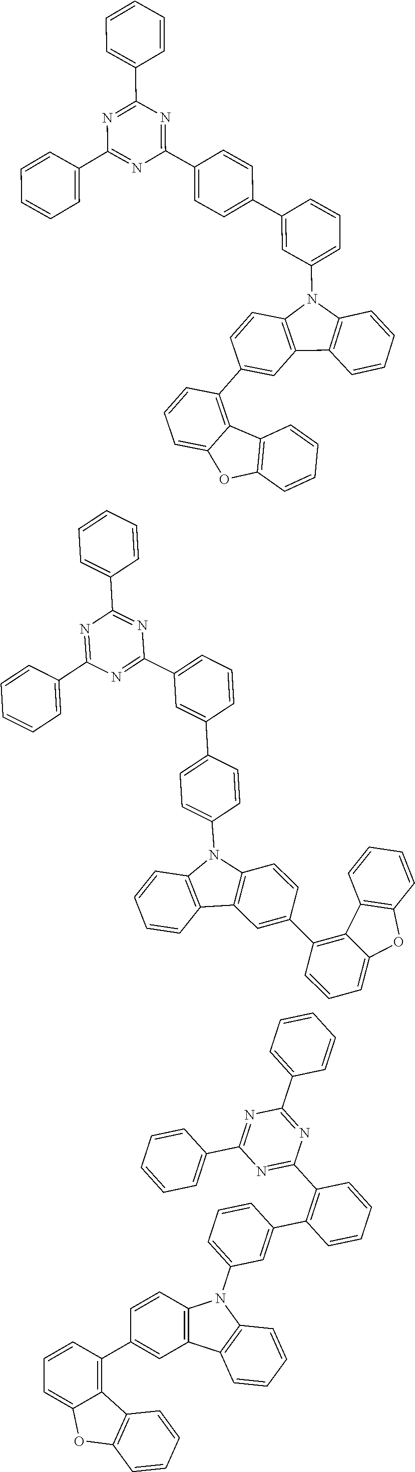

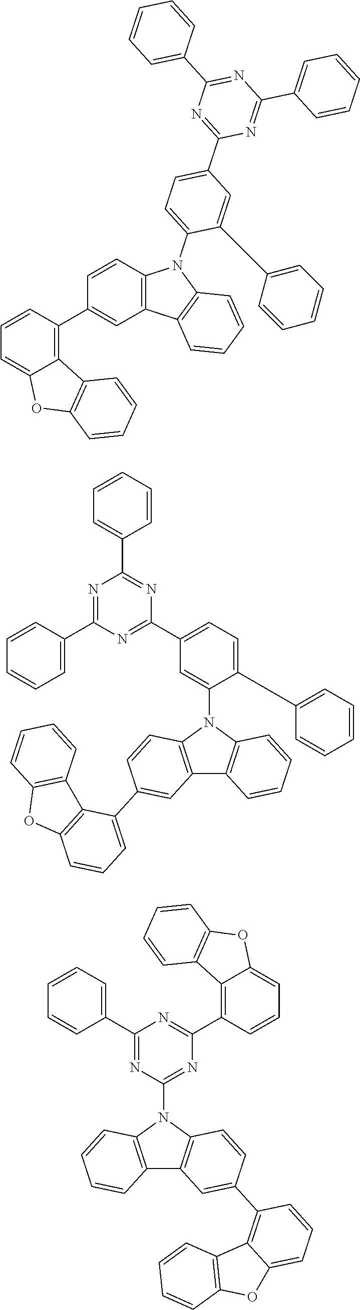

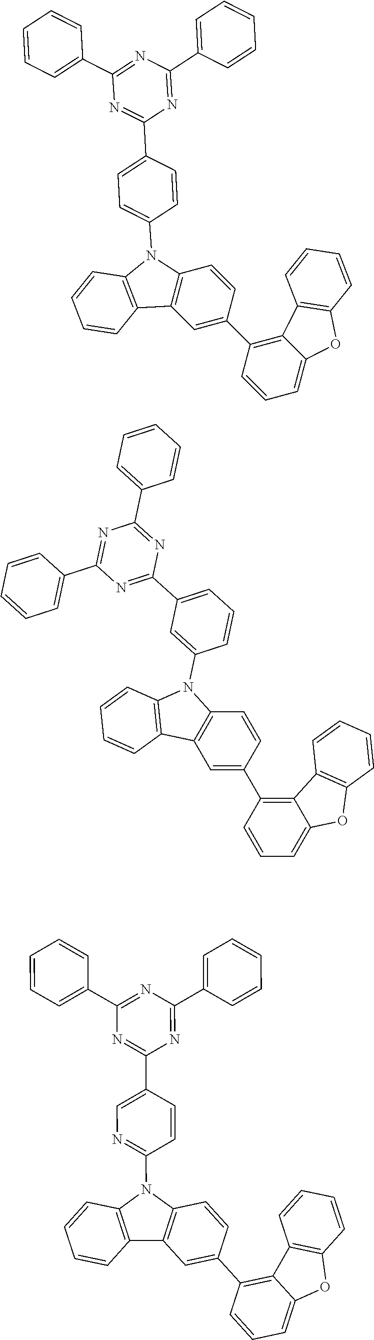 [SMILES: c1ccc(-c2nc(-c3ccccc3)nc(-c3ccc(-n4c5ccccc5c5cc(-c6cccc7oc8ccccc8c67)ccc54)cc3)n2)cc1 ; c1ccc(-c2nc(-c3ccccc3)nc(-c3cccc(-n4c5ccccc5c5cc(-c6cccc7oc8ccccc8c67)ccc54)c3)n2)cc1 ; c1ccc(-c2nc(-c3ccccc3)nc(-c3ccc(-n4c5ccccc5c5cc(-c6cccc7oc8ccccc8c67)ccc54)nc3)n2)cc1]

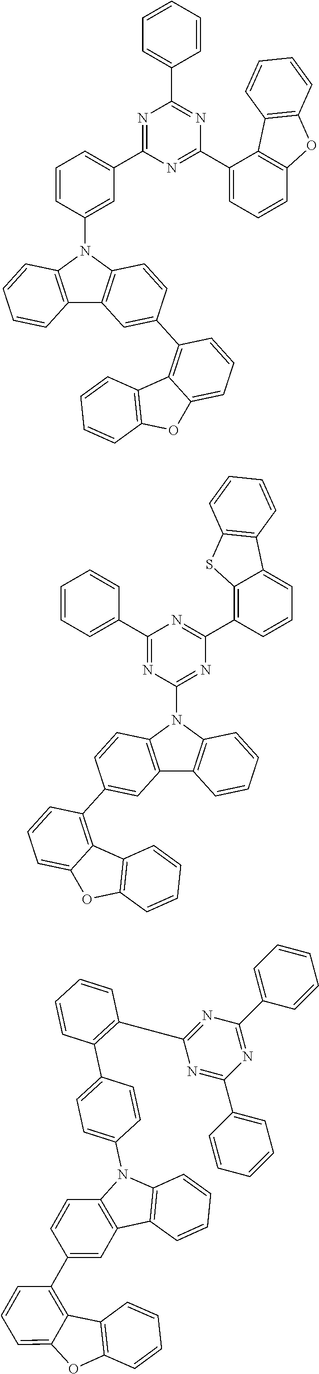 [SMILES: c1ccc(-c2nc(-c3cccc(-n4c5ccccc5c5cc(-c6cccc7oc8ccccc8c67)ccc54)c3)nc(-c3cccc4oc5ccccc5c34)n2)cc1 ; c1ccc(-c2nc(-c3cccc4c3sc3ccccc34)nc(-n3c4ccccc4c4cc(-c5cccc6oc7ccccc7c56)ccc43)n2)cc1 ; c1ccc(-c2nc(Cc3ccccc3-c3ccc(-n4c5ccccc5c5cc(-c6cccc7oc8ccccc8c67)ccc54)cc3)nc(-c3ccccc3)n2)cc1]

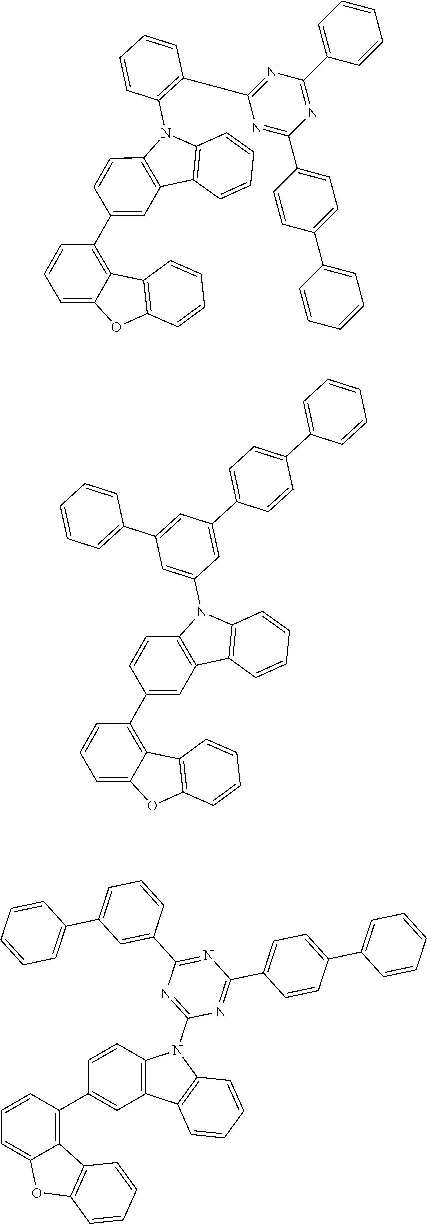 [SMILES: c1ccc(-c2ccc(-c3nc(Cc4ccccc4-n4c5ccccc5c5cc(-c6cccc7oc8ccccc8c67)ccc54)nc(-c4ccccc4)n3)cc2)cc1 ; c1ccc(-c2ccc(-c3cc(-c4ccccc4)cc(-n4c5ccccc5c5cc(-c6cccc7oc8ccccc8c67)ccc54)c3)cc2)cc1 ; c1ccc(-c2ccc(-c3nc(-c4cccc(-c5ccccc5)c4)nc(-n4c5ccccc5c5cc(-c6cccc7oc8ccccc8c67)ccc54)n3)cc2)cc1]

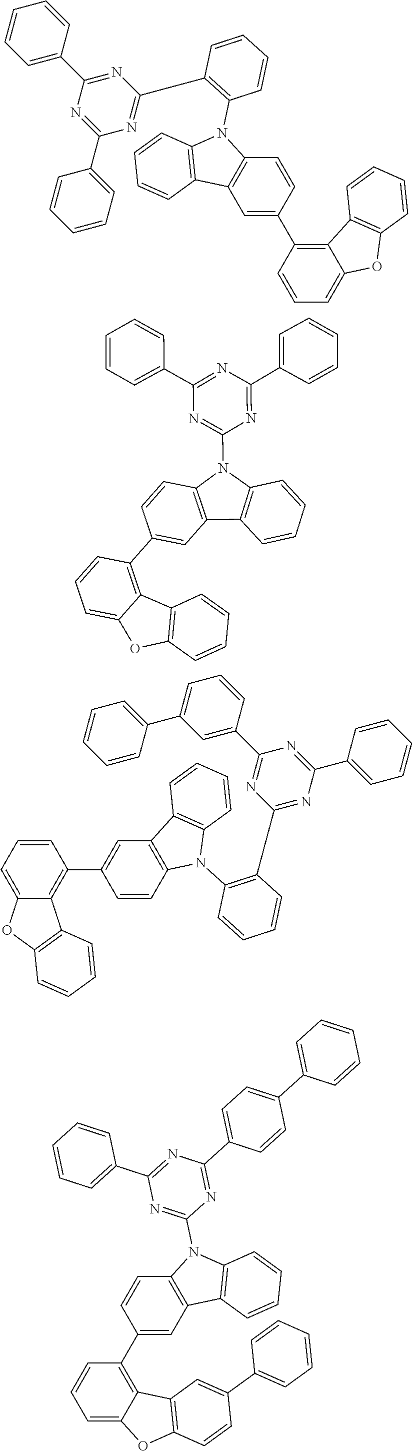

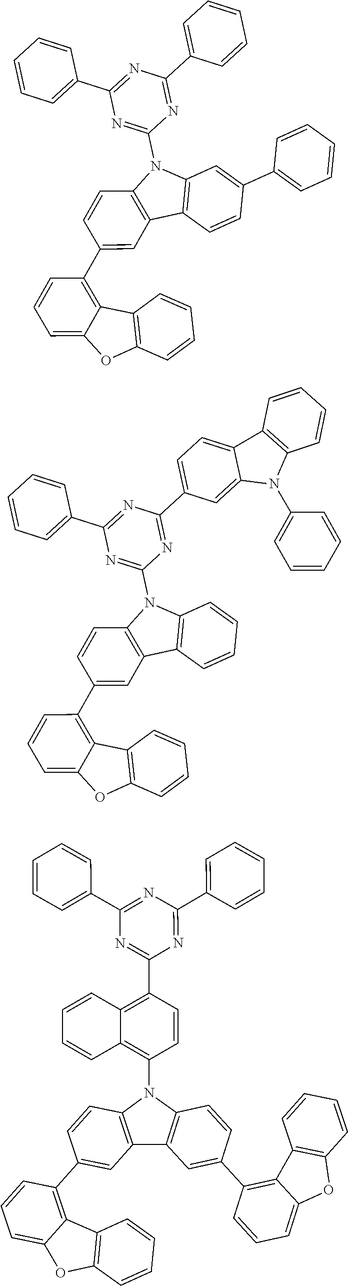 [SMILES: c1ccc(-c2ccc3c4cc(-c5cccc6oc7ccccc7c56)ccc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4cc(-c5cccc6oc7ccccc7c56)ccc43)n2)cc1 ; c1ccc(-c2nc(-c3ccccc3)nc(-c3ccc(-n4c5ccc(-c6cccc7oc8ccccc8c67)cc5c5cc(-c6cccc7oc8ccccc8c67)ccc54)c4ccccc34)n2)cc1]

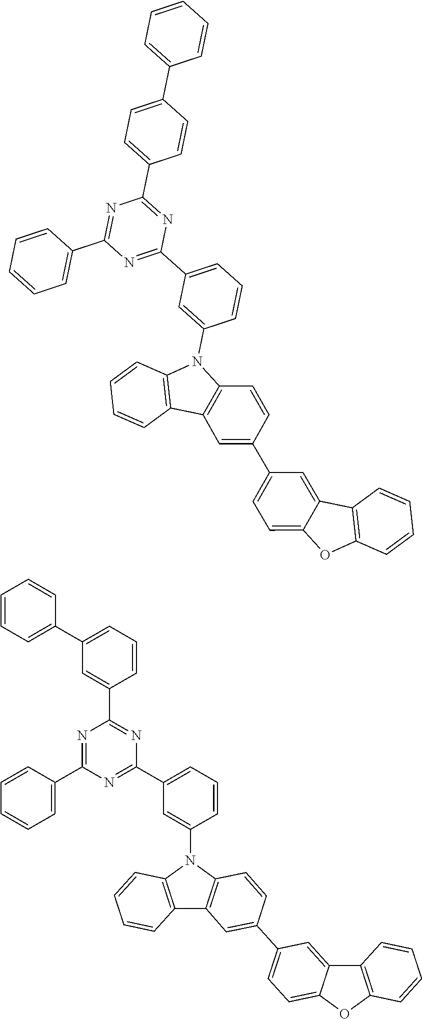 [SMILES: c1ccc(-c2ccc(-c3nc(-c4ccccc4)nc(-c4cccc(-n5c6ccccc6c6cc(-c7ccc8oc9ccccc9c8c7)ccc65)c4)n3)cc2)cc1 ; c1ccc(-c2cccc(-c3nc(-c4ccccc4)nc(-c4cccc(-n5c6ccccc6c6cc(-c7ccc8oc9ccccc9c8c7)ccc65)c4)n3)c2)cc1]

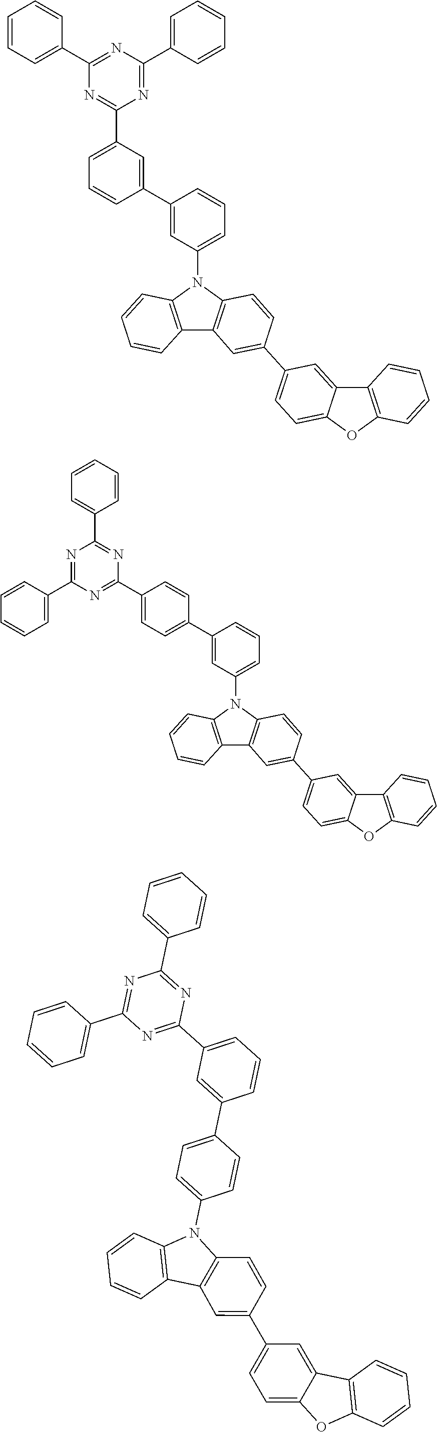 [SMILES: c1ccc(-c2nc(-c3ccccc3)nc(-c3cccc(-c4cccc(-n5c6ccccc6c6cc(-c7ccc8oc9ccccc9c8c7)ccc65)c4)c3)n2)cc1 ; c1ccc(-c2nc(-c3ccccc3)nc(-c3ccc(-c4cccc(-n5c6ccccc6c6cc(-c7ccc8oc9ccccc9c8c7)ccc65)c4)cc3)n2)cc1 ; c1ccc(-c2nc(-c3ccccc3)nc(-c3cccc(-c4ccc(-n5c6ccccc6c6cc(-c7ccc8oc9ccccc9c8c7)ccc65)cc4)c3)n2)cc1]

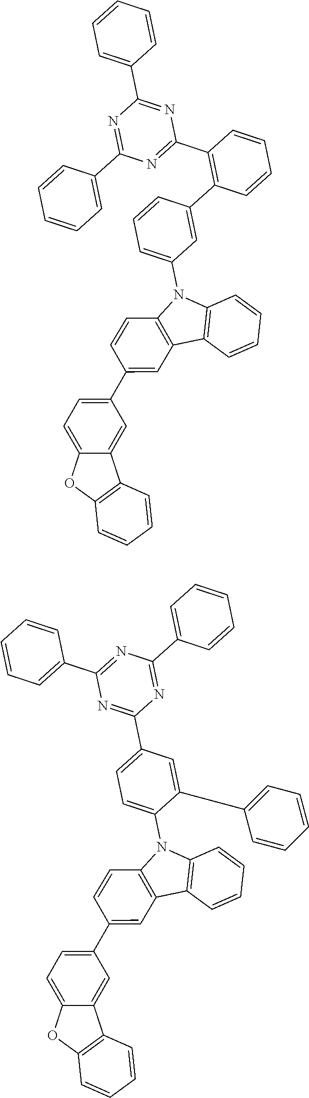 [SMILES: c1ccc(-c2nc(-c3ccccc3)nc(-c3ccccc3-c3cccc(-n4c5ccccc5c5cc(-c6ccc7oc8ccccc8c7c6)ccc54)c3)n2)cc1 ; c1ccc(Cc2cc(-c3nc(-c4ccccc4)nc(-c4ccccc4)n3)ccc2-n2c3ccccc3c3cc(-c4ccc5oc6ccccc6c5c4)ccc32)cc1]

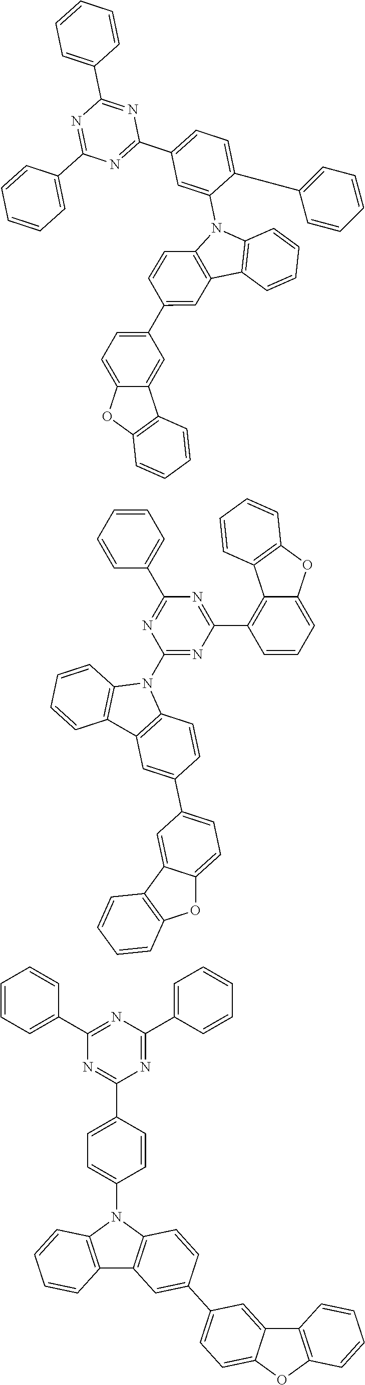

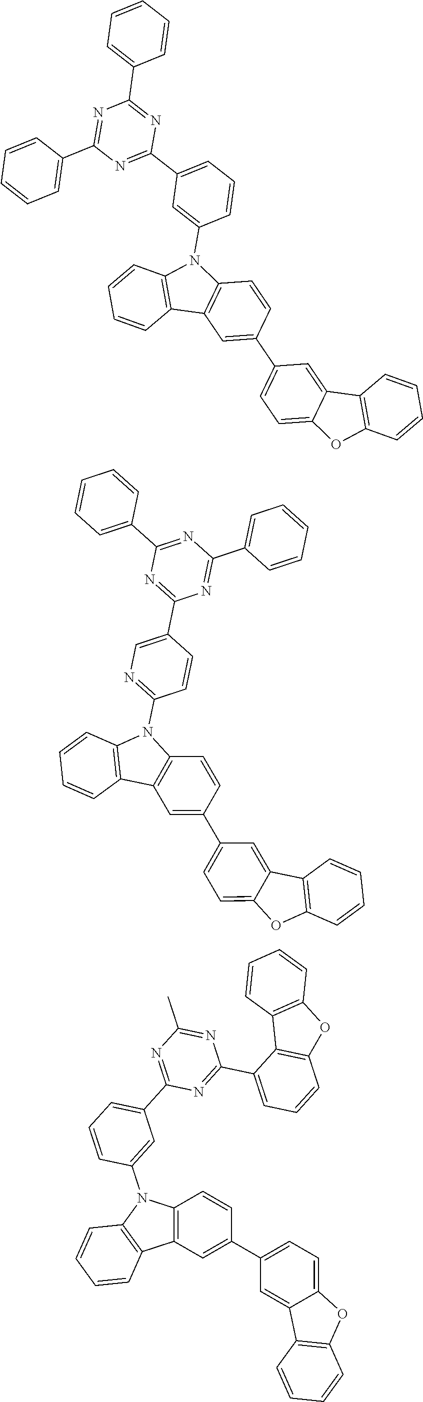

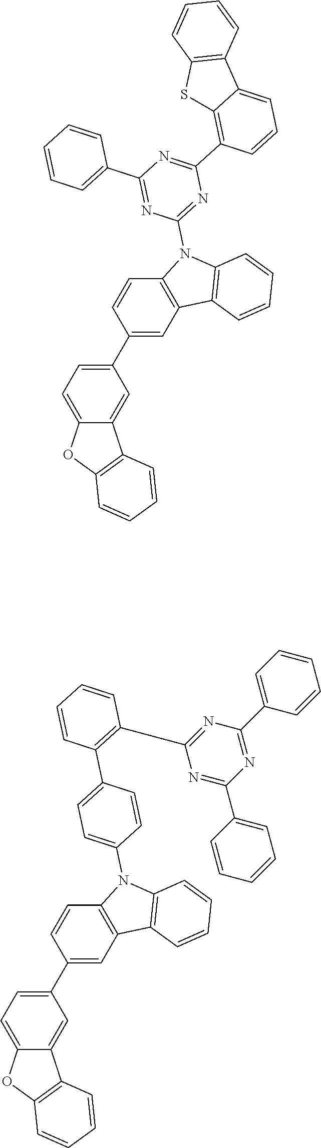 [SMILES: c1ccc(-c2nc(-c3cccc4c3sc3ccccc34)nc(-n3c4ccccc4c4cc(-c5ccc6oc7ccccc7c6c5)ccc43)n2)cc1 ; c1ccc(-c2nc(Cc3ccccc3-c3ccc(-n4c5ccccc5c5cc(-c6ccc7oc8ccccc8c7c6)ccc54)cc3)nc(-c3ccccc3)n2)cc1]

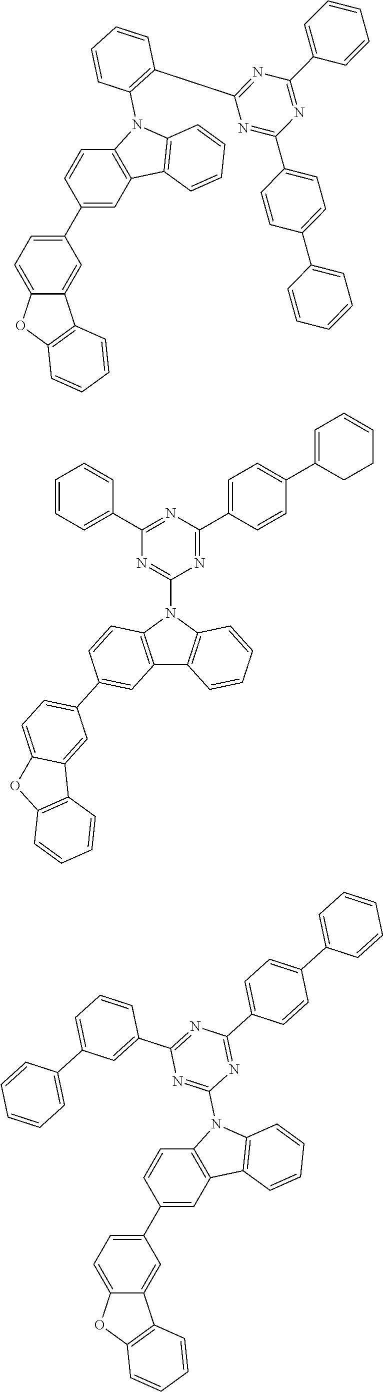 [SMILES: c1ccc(-c2ccc(-c3nc(-c4ccccc4)nc(-c4ccccc4-n4c5ccccc5c5cc(-c6ccc7oc8ccccc8c7c6)ccc54)n3)cc2)cc1 ; C1=CCCC(c2ccc(-c3nc(-c4ccccc4)nc(-n4c5ccccc5c5cc(-c6ccc7oc8ccccc8c7c6)ccc54)n3)cc2)=C1 ; c1ccc(-c2ccc(-c3nc(-c4cccc(-c5ccccc5)c4)nc(-n4c5ccccc5c5cc(-c6ccc7oc8ccccc8c7c6)ccc54)n3)cc2)cc1]

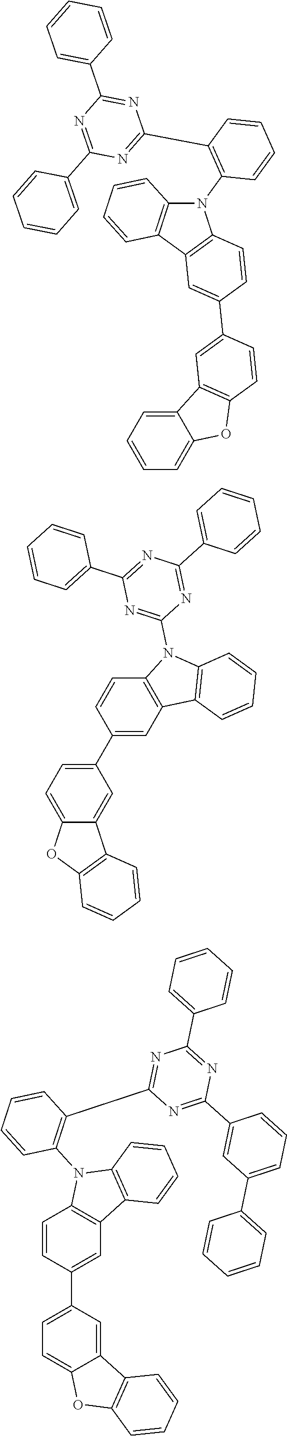

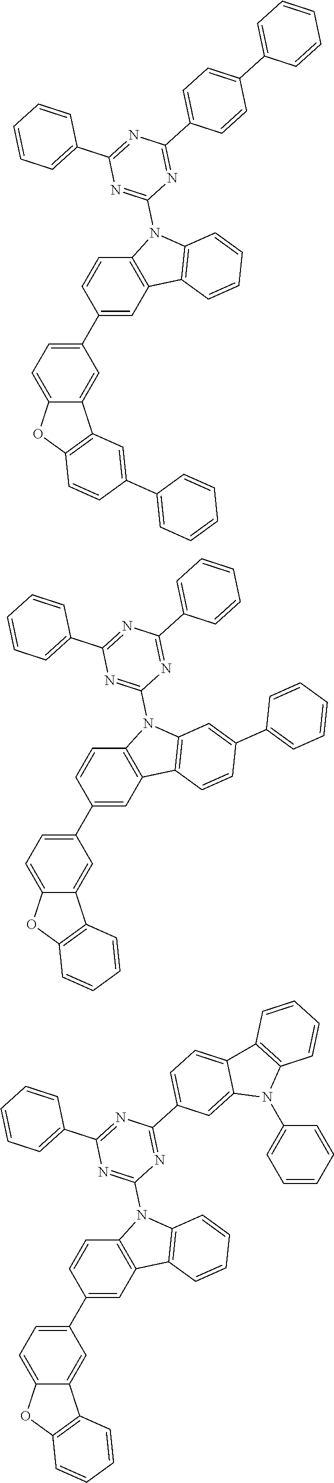

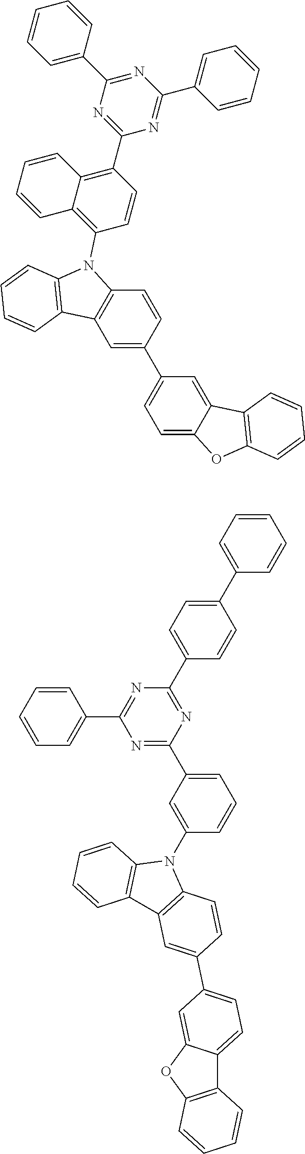 [SMILES: c1ccc(-c2nc(-c3ccccc3)nc(-c3ccc(-n4c5ccccc5c5cc(-c6ccc7oc8ccccc8c7c6)ccc54)c4ccccc34)n2)cc1 ; c1ccc(-c2ccc(-c3nc(-c4ccccc4)nc(-c4cccc(-n5c6ccccc6c6cc(-c7ccc8c(c7)oc7ccccc78)ccc65)c4)n3)cc2)cc1]

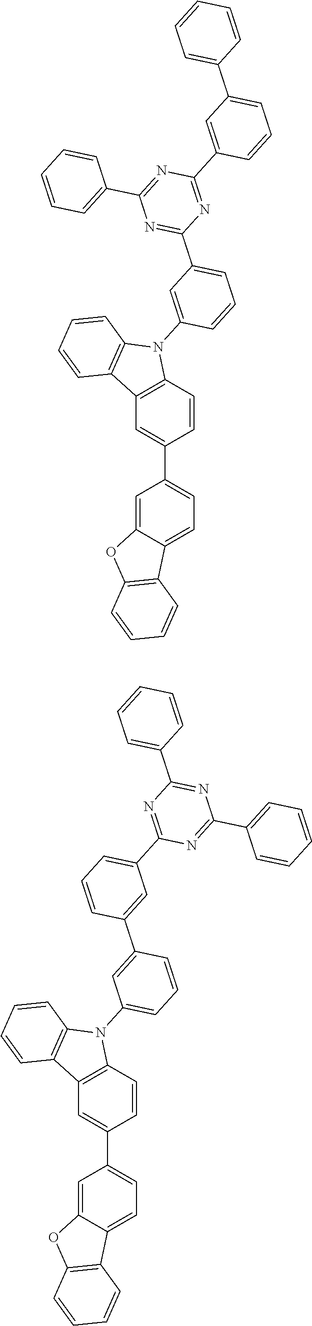 [SMILES: c1ccc(-c2cccc(-c3nc(-c4ccccc4)nc(-c4cccc(-n5c6ccccc6c6cc(-c7ccc8c(c7)oc7ccccc78)ccc65)c4)n3)c2)cc1 ; c1ccc(-c2nc(-c3ccccc3)nc(-c3cccc(-c4cccc(-n5c6ccccc6c6cc(-c7ccc8c(c7)oc7ccccc78)ccc65)c4)c3)n2)cc1]

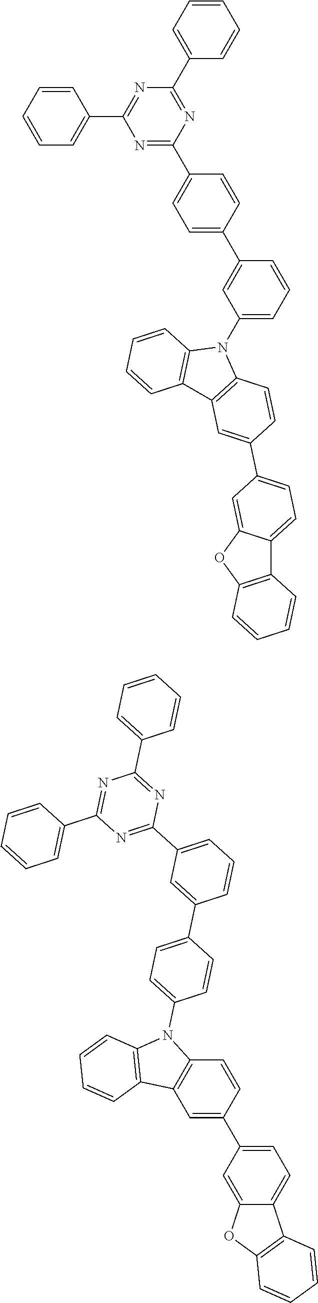 [SMILES: c1ccc(-c2nc(-c3ccccc3)nc(-c3ccc(-c4cccc(-n5c6ccccc6c6cc(-c7ccc8c(c7)oc7ccccc78)ccc65)c4)cc3)n2)cc1 ; c1ccc(-c2nc(-c3ccccc3)nc(-c3cccc(-c4ccc(-n5c6ccccc6c6cc(-c7ccc8c(c7)oc7ccccc78)ccc65)cc4)c3)n2)cc1]

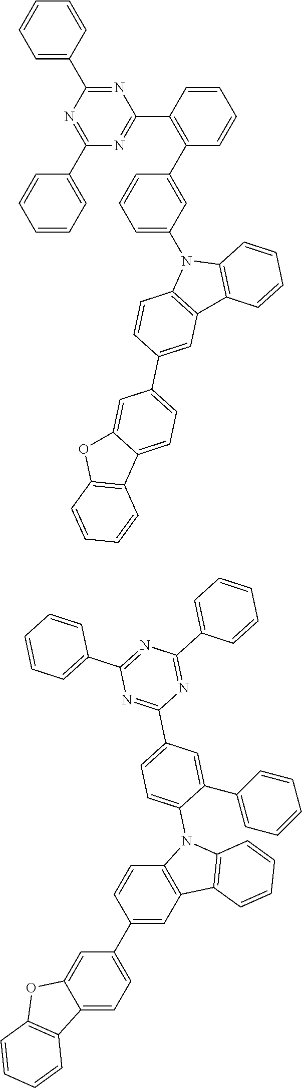 [SMILES: c1ccc(-c2nc(-c3ccccc3)nc(-c3ccccc3-c3cccc(-n4c5ccccc5c5cc(-c6ccc7c(c6)oc6ccccc67)ccc54)c3)n2)cc1 ; c1ccc(-c2nc(-c3ccccc3)nc(-c3ccc(-n4c5ccccc5c5cc(-c6ccc7c(c6)oc6ccccc67)ccc54)c(-c4ccccc4)c3)n2)cc1]

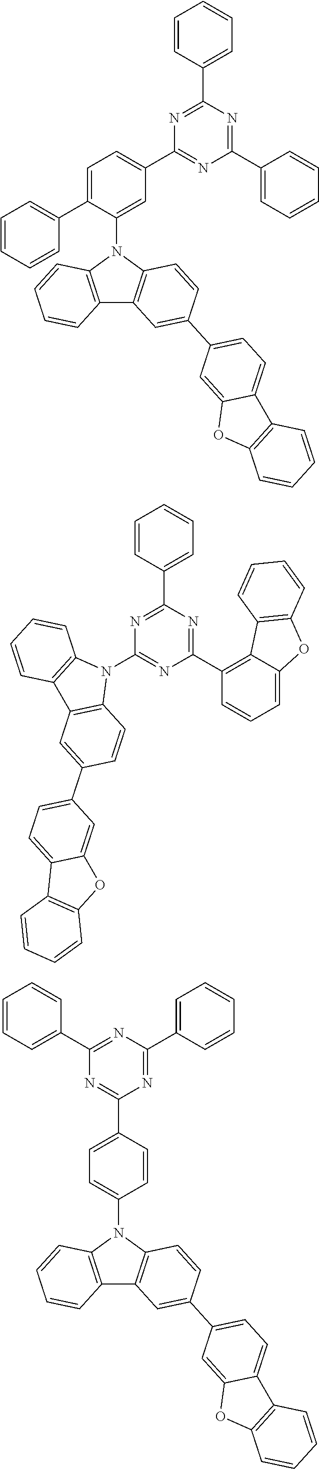

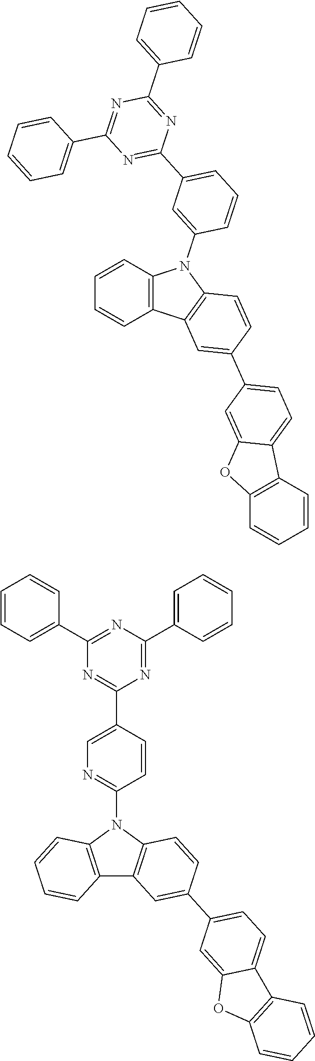 [SMILES: c1ccc(-c2nc(-c3ccccc3)nc(-c3cccc(-n4c5ccccc5c5cc(-c6ccc7c(c6)oc6ccccc67)ccc54)c3)n2)cc1 ; c1ccc(-c2nc(-c3ccccc3)nc(-c3ccc(-n4c5ccccc5c5cc(-c6ccc7c(c6)oc6ccccc67)ccc54)nc3)n2)cc1]

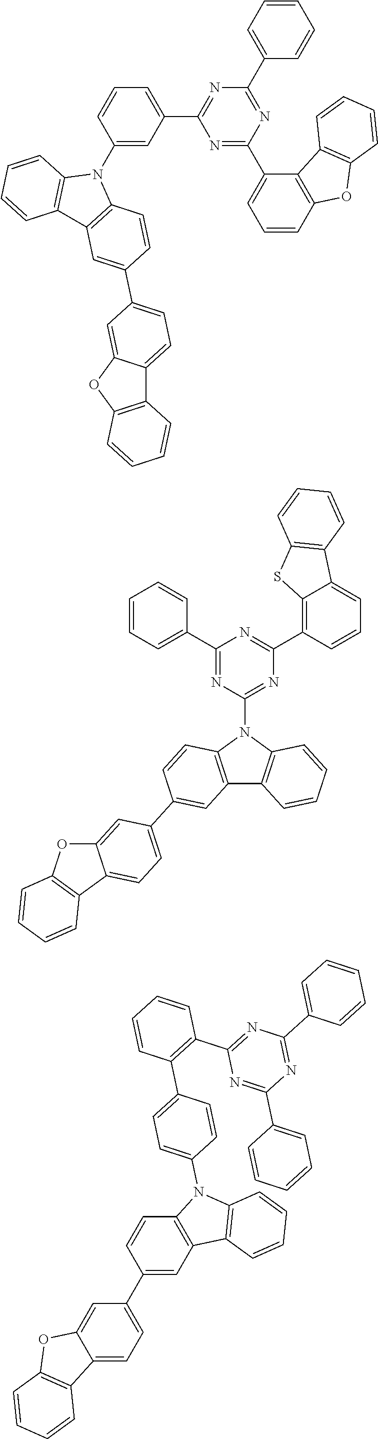 [SMILES: c1ccc(-c2nc(-c3cccc(-n4c5ccccc5c5cc(-c6ccc7c(c6)oc6ccccc67)ccc54)c3)nc(-c3cccc4oc5ccccc5c34)n2)cc1 ; c1ccc(-c2nc(-c3cccc4c3sc3ccccc34)nc(-n3c4ccccc4c4cc(-c5ccc6c(c5)oc5ccccc56)ccc43)n2)cc1 ; c1ccc(-c2nc(-c3ccccc3)nc(-c3ccccc3-c3ccc(-n4c5ccccc5c5cc(-c6ccc7c(c6)oc6ccccc67)ccc54)cc3)n2)cc1]

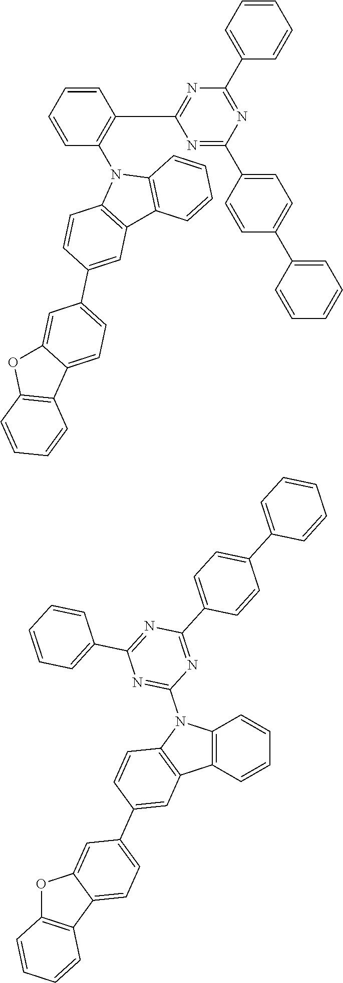 [SMILES: c1ccc(-c2ccc(-c3nc(-c4ccccc4)nc(-c4ccccc4-n4c5ccccc5c5cc(-c6ccc7c(c6)oc6ccccc67)ccc54)n3)cc2)cc1 ; c1ccc(-c2ccc(-c3nc(-c4ccccc4)nc(-n4c5ccccc5c5cc(-c6ccc7c(c6)oc6ccccc67)ccc54)n3)cc2)cc1]

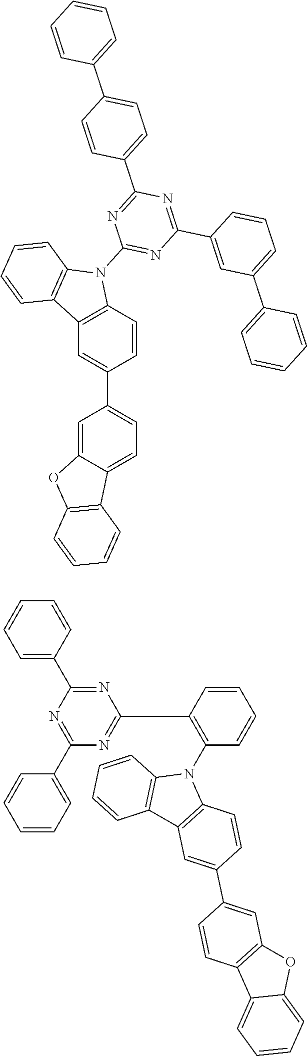

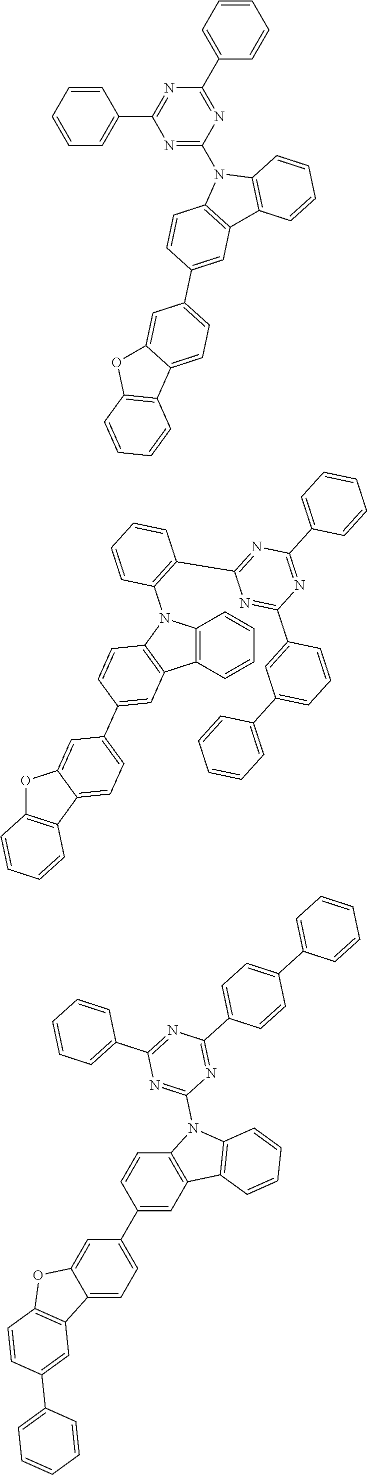 [SMILES: c1ccc(-c2nc(-c3ccccc3)nc(-n3c4ccccc4c4cc(-c5ccc6c(c5)oc5ccccc56)ccc43)n2)cc1 ; c1ccc(-c2cccc(-c3nc(-c4ccccc4)nc(-c4ccccc4-n4c5ccccc5c5cc(-c6ccc7c(c6)oc6ccccc67)ccc54)n3)c2)cc1 ; c1ccc(-c2ccc(-c3nc(-c4ccccc4)nc(-n4c5ccccc5c5cc(-c6ccc7c(c6)oc6ccc(-c8ccccc8)cc67)ccc54)n3)cc2)cc1]

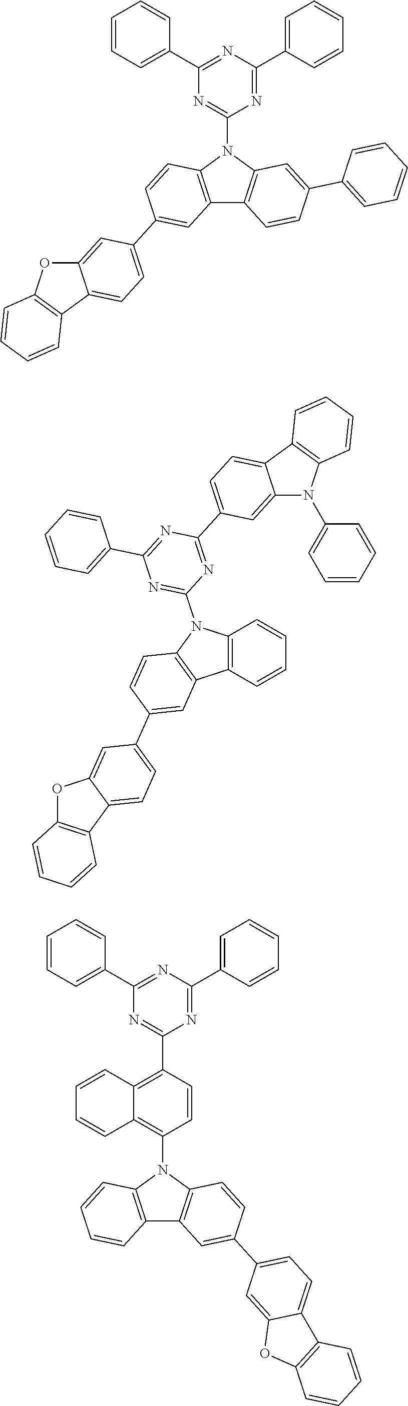

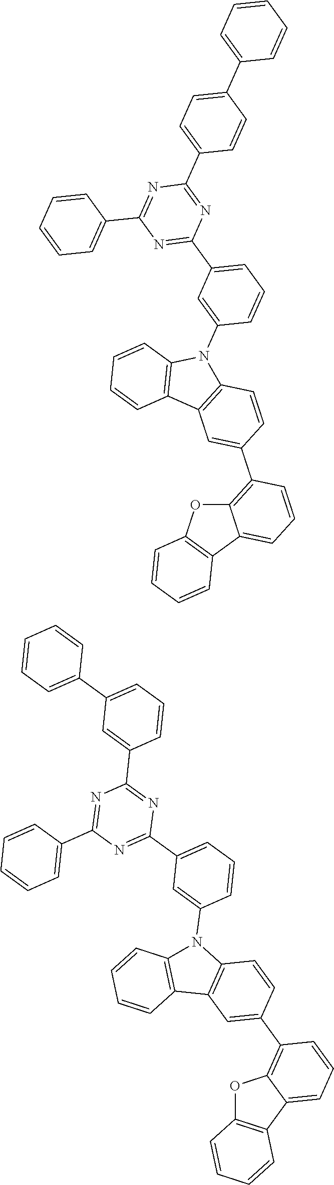 [SMILES: c1ccc(-c2ccc(-c3nc(-c4ccccc4)nc(-c4cccc(-n5c6ccccc6c6cc(-c7cccc8c7oc7ccccc78)ccc65)c4)n3)cc2)cc1 ; c1ccc(-c2cccc(-c3nc(-c4ccccc4)nc(-c4cccc(-n5c6ccccc6c6cc(-c7cccc8c7oc7ccccc78)ccc65)c4)n3)c2)cc1]

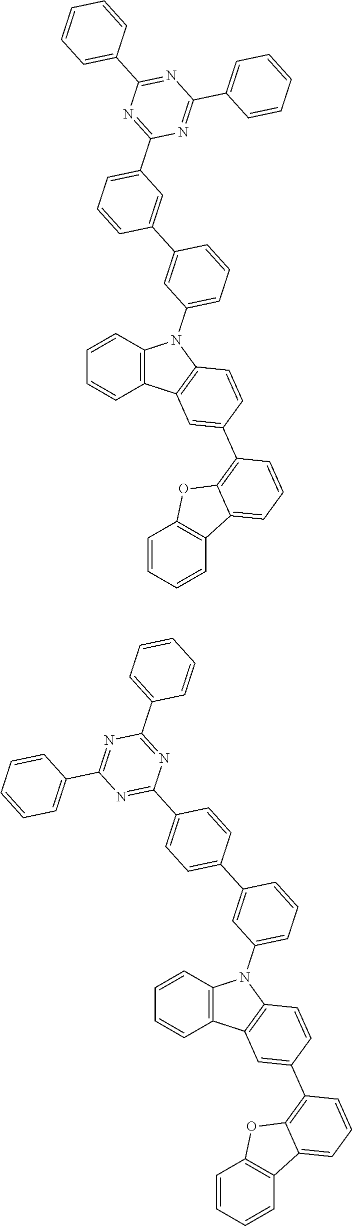 [SMILES: c1ccc(-c2nc(-c3ccccc3)nc(-c3cccc(-c4cccc(-n5c6ccccc6c6cc(-c7cccc8c7oc7ccccc78)ccc65)c4)c3)n2)cc1 ; c1ccc(-c2nc(-c3ccccc3)nc(-c3ccc(-c4cccc(-n5c6ccccc6c6cc(-c7cccc8c7oc7ccccc78)ccc65)c4)cc3)n2)cc1]

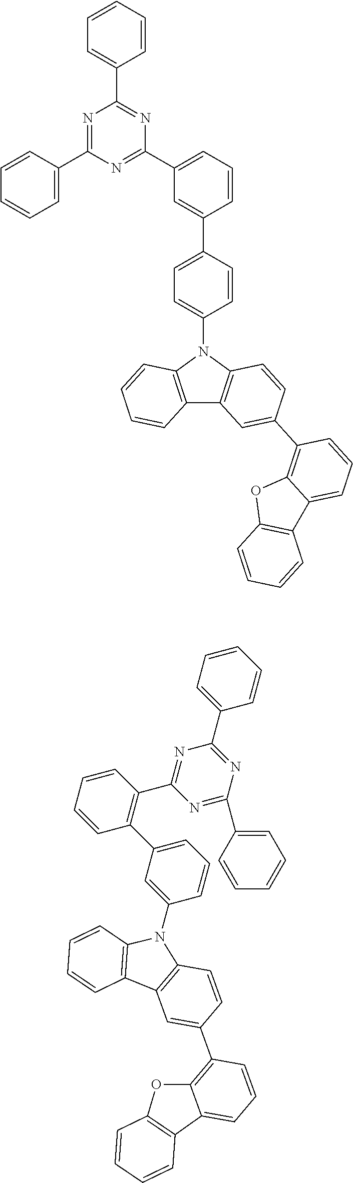 [SMILES: c1ccc(-c2nc(-c3ccccc3)nc(-c3cccc(-c4ccc(-n5c6ccccc6c6cc(-c7cccc8c7oc7ccccc78)ccc65)cc4)c3)n2)cc1 ; c1ccc(-c2nc(-c3ccccc3)nc(-c3ccccc3-c3cccc(-n4c5ccccc5c5cc(-c6cccc7c6oc6ccccc67)ccc54)c3)n2)cc1]

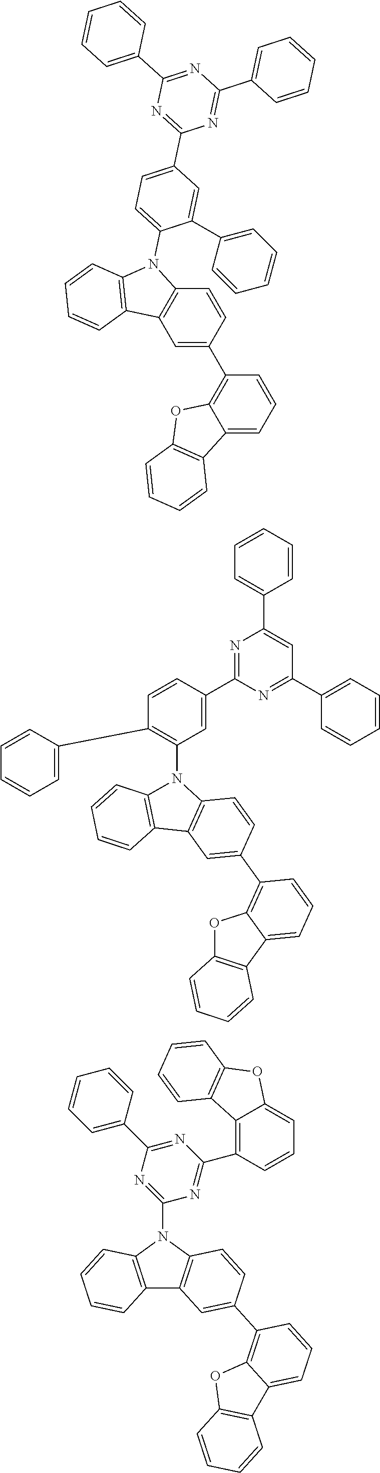

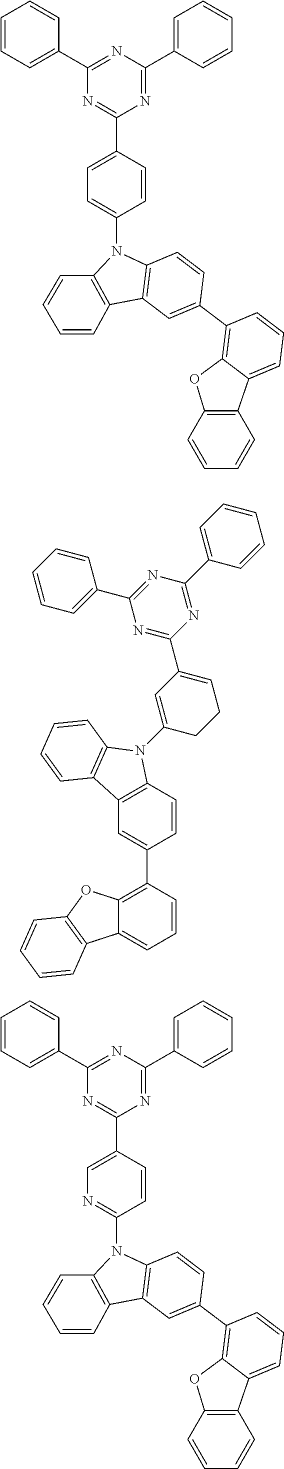

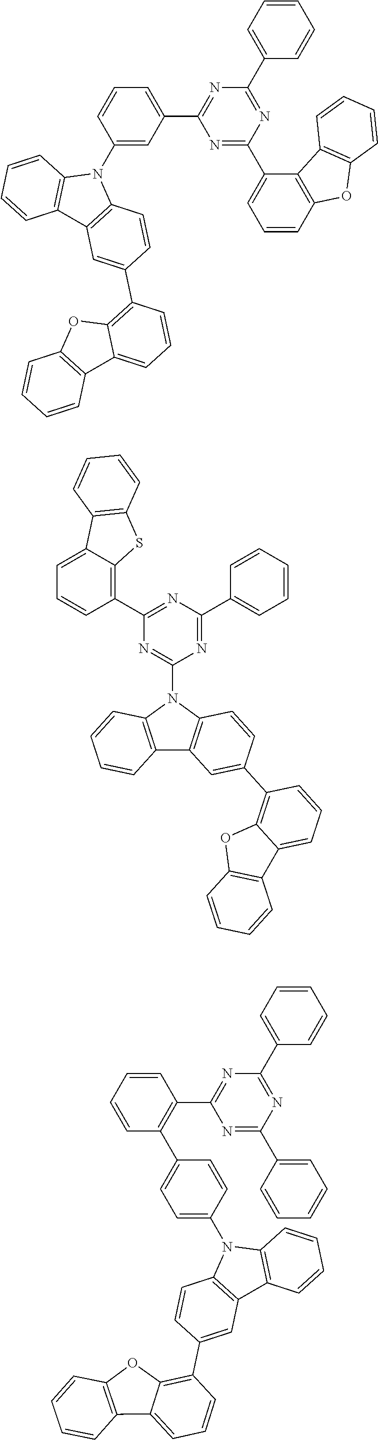 [SMILES: c1ccc(-c2nc(-c3cccc(-n4c5ccccc5c5cc(-c6cccc7c6oc6ccccc67)ccc54)c3)nc(-c3cccc4oc5ccccc5c34)n2)cc1 ; c1ccc(-c2nc(-c3cccc4c3sc3ccccc34)nc(-n3c4ccccc4c4cc(-c5cccc6c5oc5ccccc56)ccc43)n2)cc1 ; c1ccc(-c2nc(-c3ccccc3)nc(-c3ccccc3-c3ccc(-n4c5ccccc5c5cc(-c6cccc7c6oc6ccccc67)ccc54)cc3)n2)cc1]

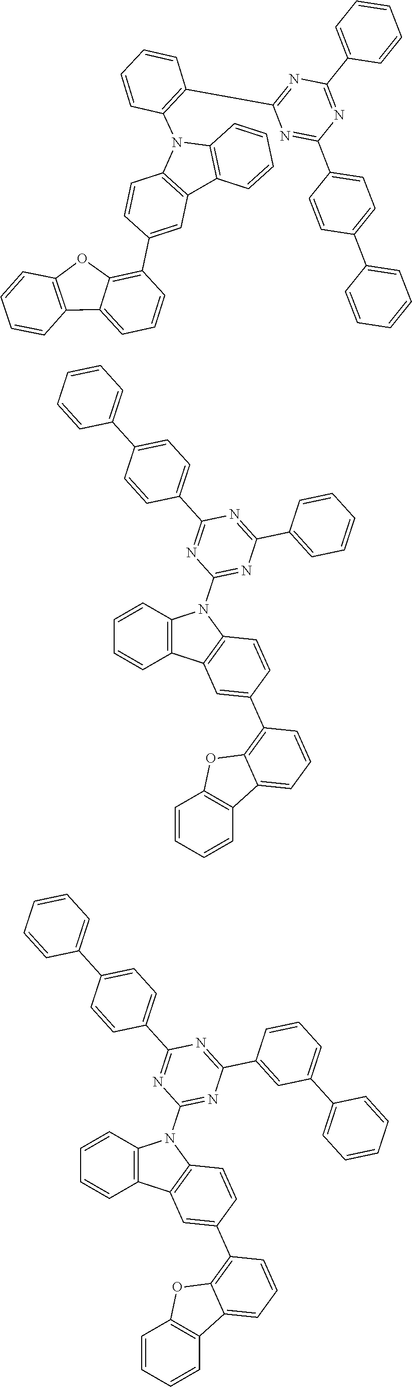

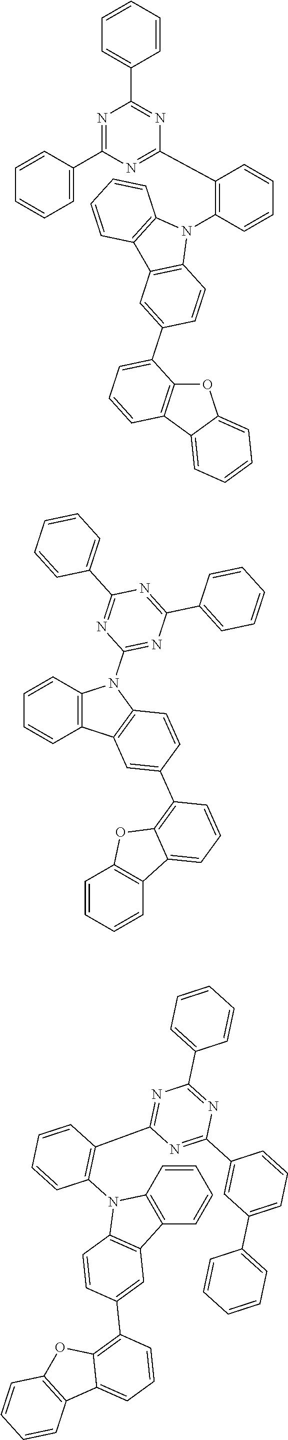 [SMILES: c1ccc(-c2nc(-c3ccccc3)nc(-c3ccccc3-n3c4ccccc4c4cc(-c5cccc6c5oc5ccccc56)ccc43)n2)cc1 ; c1ccc(-c2nc(-c3ccccc3)nc(-n3c4ccccc4c4cc(-c5cccc6c5oc5ccccc56)ccc43)n2)cc1 ; c1ccc(-c2cccc(-c3nc(-c4ccccc4)nc(-c4ccccc4-n4c5ccccc5c5cc(-c6cccc7c6oc6ccccc67)ccc54)n3)c2)cc1]

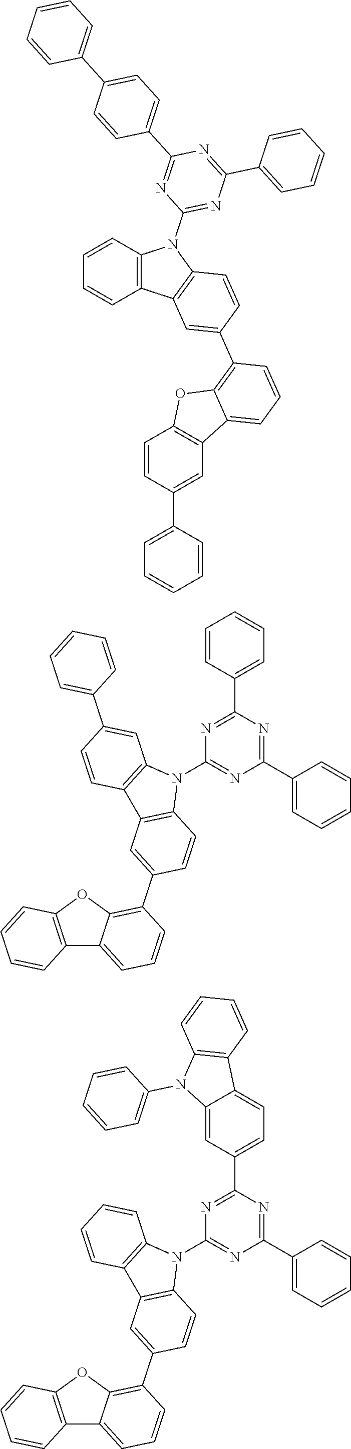 [SMILES: c1ccc(-c2ccc(-c3nc(-c4ccccc4)nc(-n4c5ccccc5c5cc(-c6cccc7c6oc6ccc(-c8ccccc8)cc67)ccc54)n3)cc2)cc1 ; c1ccc(-c2ccc3c4cc(-c5cccc6c5oc5ccccc56)ccc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4cc(-c5cccc6c5oc5ccccc56)ccc43)n2)cc1]

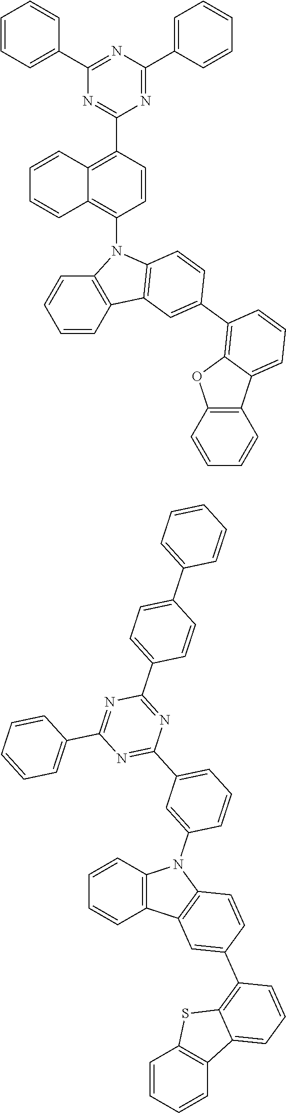

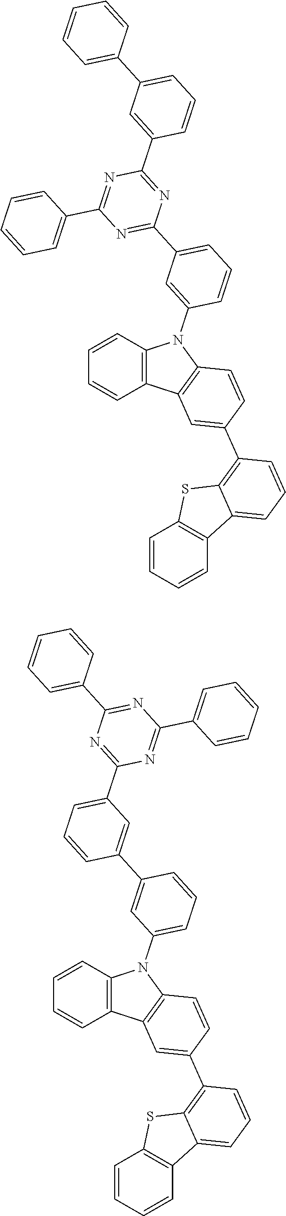 [SMILES: c1ccc(-c2cccc(-c3nc(-c4ccccc4)nc(-c4cccc(-n5c6ccccc6c6cc(-c7cccc8c7sc7ccccc78)ccc65)c4)n3)c2)cc1 ; c1ccc(-c2nc(-c3ccccc3)nc(-c3cccc(-c4cccc(-n5c6ccccc6c6cc(-c7cccc8c7sc7ccccc78)ccc65)c4)c3)n2)cc1]

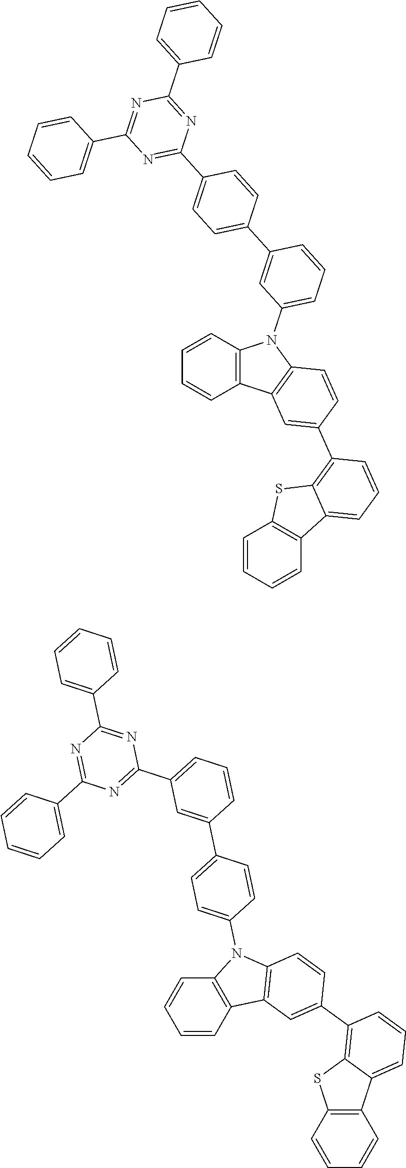 [SMILES: c1ccc(-c2nc(-c3ccccc3)nc(-c3ccc(-c4cccc(-n5c6ccccc6c6cc(-c7cccc8c7sc7ccccc78)ccc65)c4)cc3)n2)cc1 ; c1ccc(-c2nc(-c3ccccc3)nc(-c3cccc(-c4ccc(-n5c6ccccc6c6cc(-c7cccc8c7sc7ccccc78)ccc65)cc4)c3)n2)cc1]

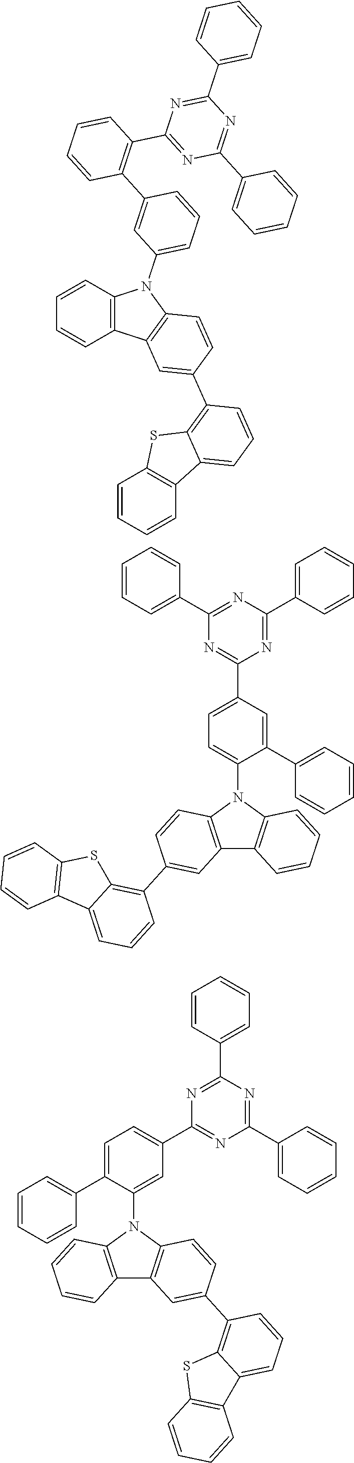

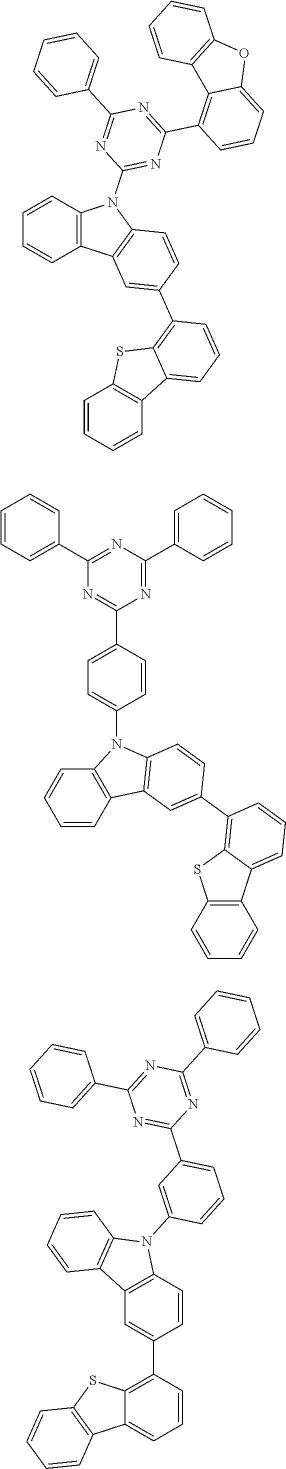

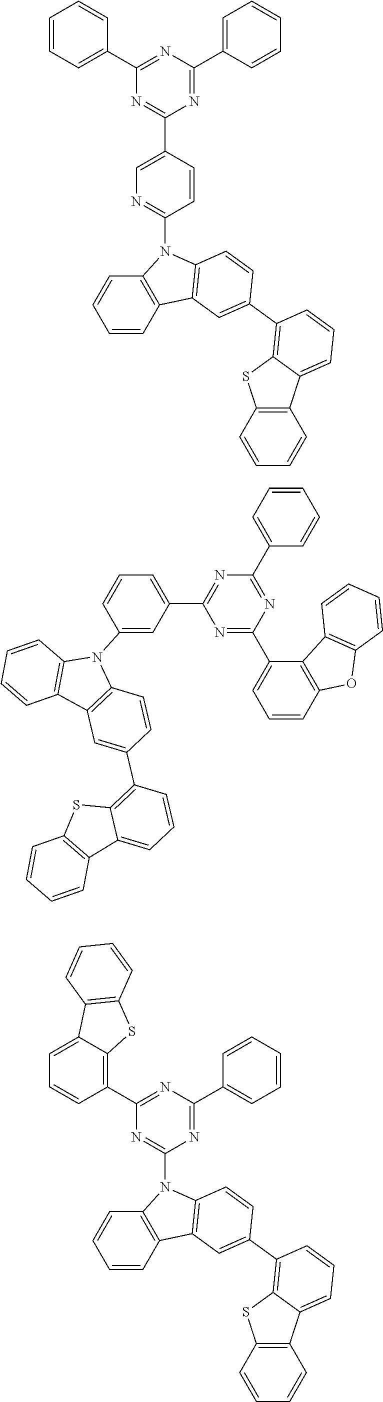

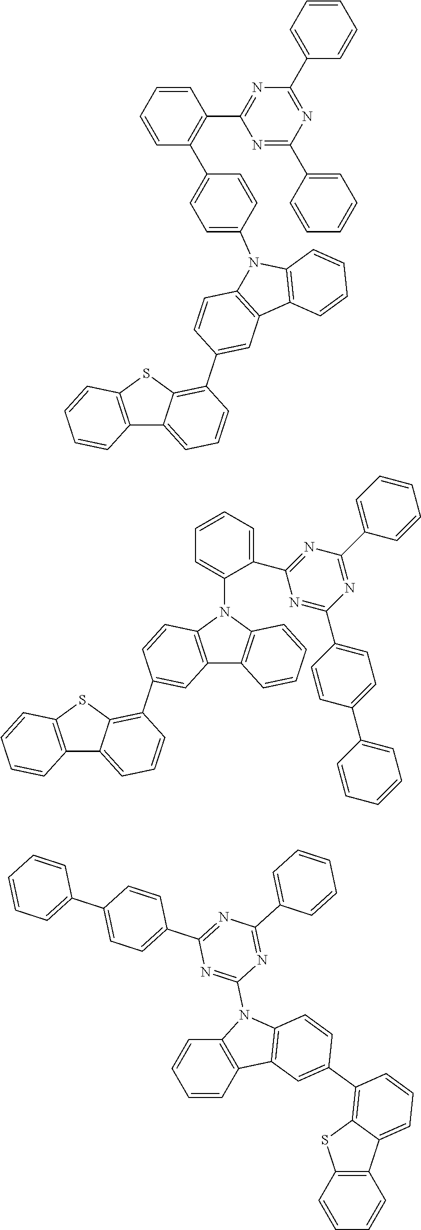 [SMILES: c1ccc(-c2nc(-c3ccccc3)nc(-c3ccccc3-c3ccc(-n4c5ccccc5c5cc(-c6cccc7c6sc6ccccc67)ccc54)cc3)n2)cc1 ; c1ccc(-c2ccc(-c3nc(-c4ccccc4)nc(-c4ccccc4-n4c5ccccc5c5cc(-c6cccc7c6sc6ccccc67)ccc54)n3)cc2)cc1 ; c1ccc(-c2ccc(-c3nc(-c4ccccc4)nc(-n4c5ccccc5c5cc(-c6cccc7c6sc6ccccc67)ccc54)n3)cc2)cc1]

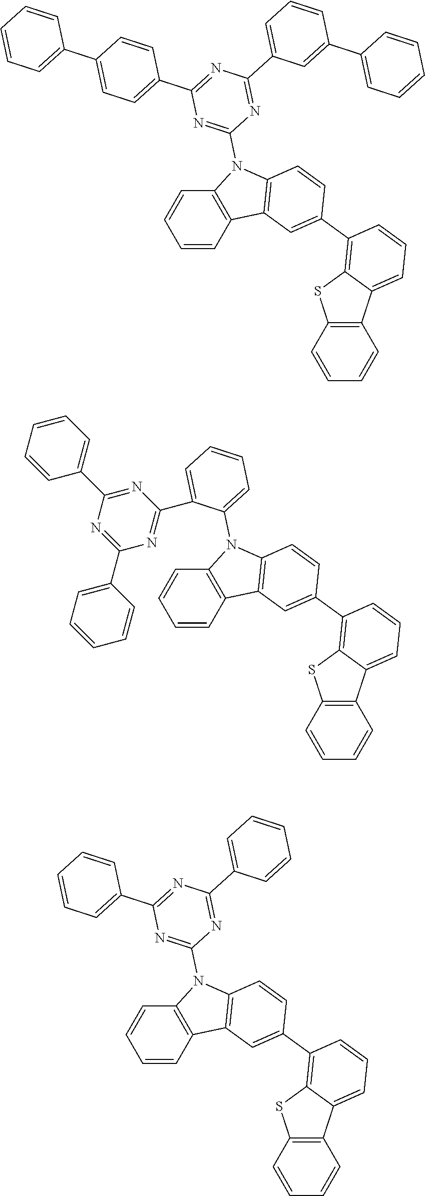 [SMILES: c1ccc(-c2ccc(-c3nc(-c4cccc(-c5ccccc5)c4)nc(-n4c5ccccc5c5cc(-c6cccc7c6sc6ccccc67)ccc54)n3)cc2)cc1 ; c1ccc(-c2nc(-c3ccccc3)nc(-c3ccccc3-n3c4ccccc4c4cc(-c5cccc6c5sc5ccccc56)ccc43)n2)cc1 ; c1ccc(-c2nc(-c3ccccc3)nc(-n3c4ccccc4c4cc(-c5cccc6c5sc5ccccc56)ccc43)n2)cc1]

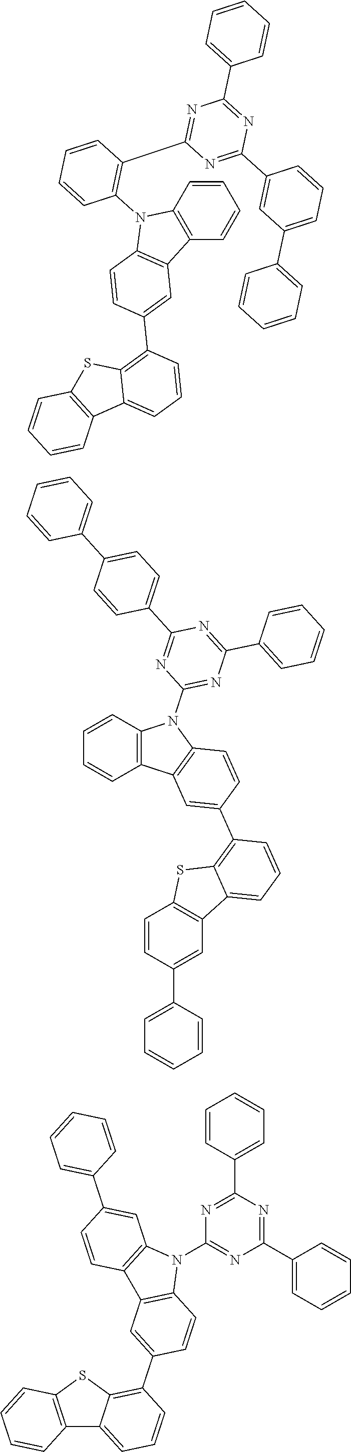

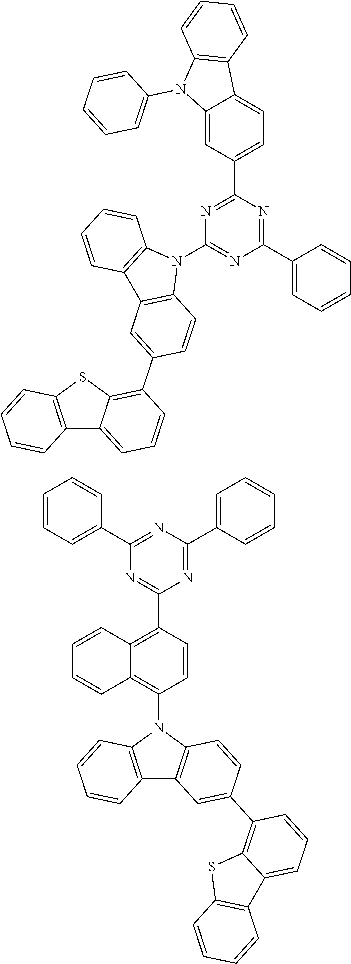

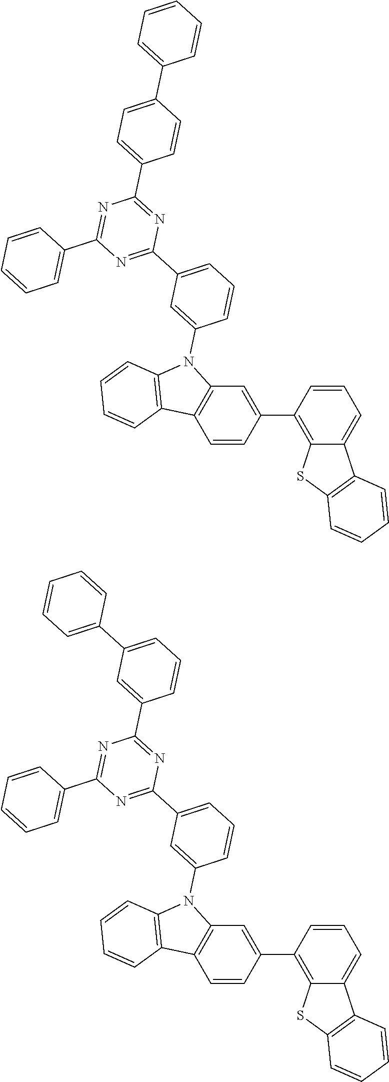 [SMILES: c1ccc(-c2ccc(-c3nc(-c4ccccc4)nc(-c4cccc(-n5c6ccccc6c6ccc(-c7cccc8c7sc7ccccc78)cc65)c4)n3)cc2)cc1 ; c1ccc(-c2cccc(-c3nc(-c4ccccc4)nc(-c4cccc(-n5c6ccccc6c6ccc(-c7cccc8c7sc7ccccc78)cc65)c4)n3)c2)cc1]

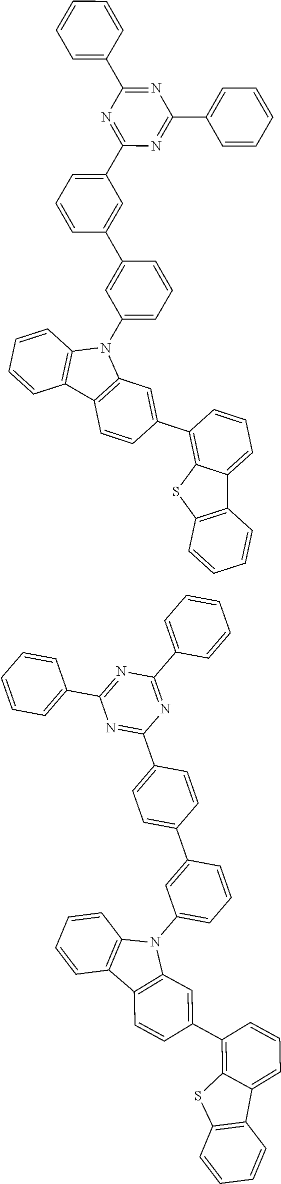

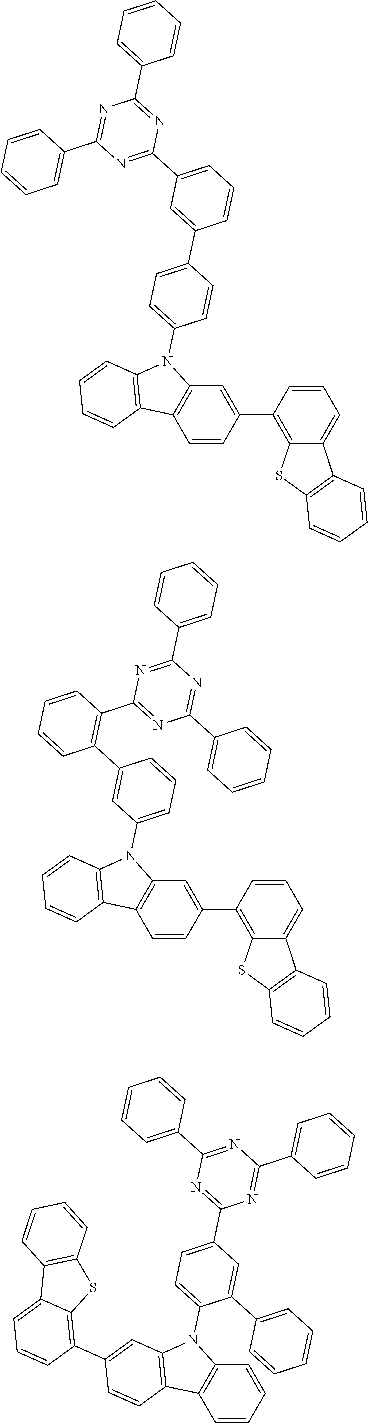 [SMILES: c1ccc(-c2nc(-c3ccccc3)nc(-c3cccc(-c4ccc(-n5c6ccccc6c6ccc(-c7cccc8c7sc7ccccc78)cc65)cc4)c3)n2)cc1 ; c1ccc(-c2nc(-c3ccccc3)nc(-c3ccccc3-c3cccc(-n4c5ccccc5c5ccc(-c6cccc7c6sc6ccccc67)cc54)c3)n2)cc1 ; c1ccc(-c2nc(-c3ccccc3)nc(-c3ccc(-n4c5ccccc5c5ccc(-c6cccc7c6sc6ccccc67)cc54)c(-c4ccccc4)c3)n2)cc1]

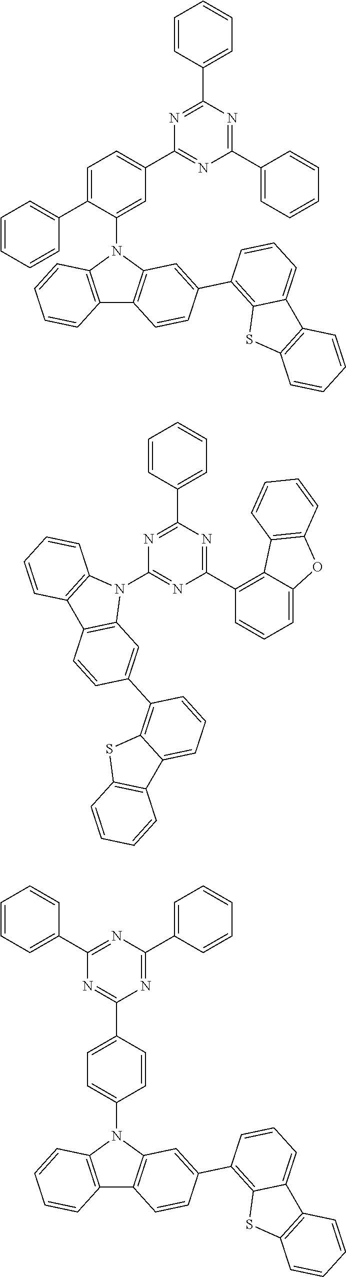 [SMILES: c1ccc(-c2nc(-c3ccccc3)nc(-c3ccc(-c4ccccc4)c(-n4c5ccccc5c5ccc(-c6cccc7c6sc6ccccc67)cc54)c3)n2)cc1 ; c1ccc(-c2nc(-c3cccc4oc5ccccc5c34)nc(-n3c4ccccc4c4ccc(-c5cccc6c5sc5ccccc56)cc43)n2)cc1 ; c1ccc(-c2nc(-c3ccccc3)nc(-c3ccc(-n4c5ccccc5c5ccc(-c6cccc7c6sc6ccccc67)cc54)cc3)n2)cc1]

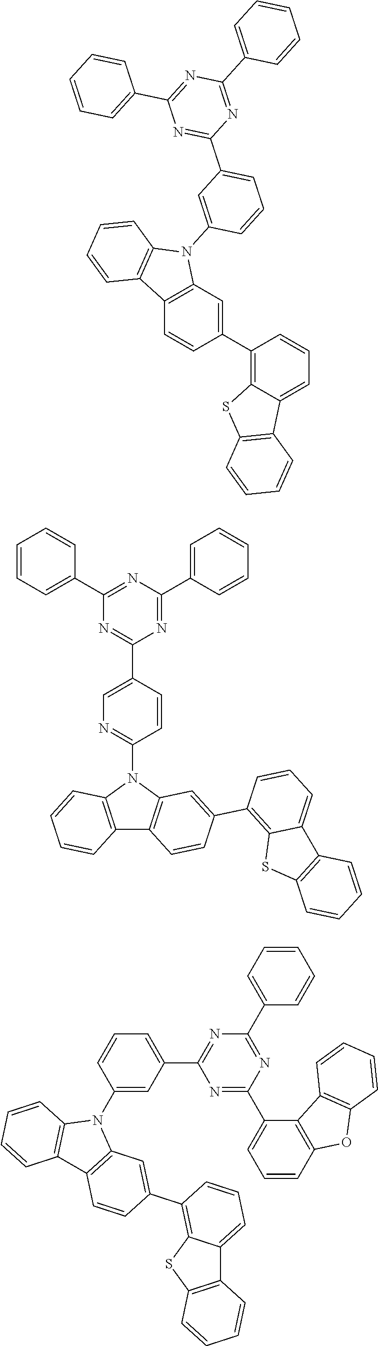

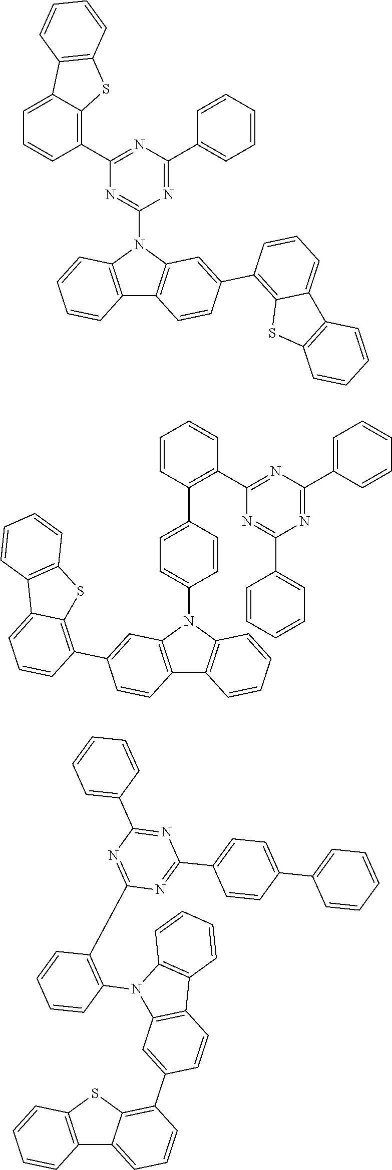

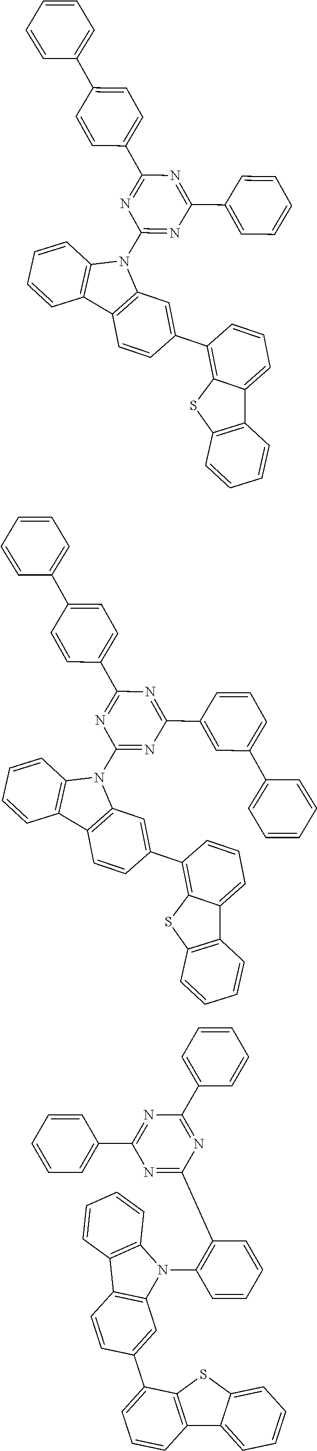

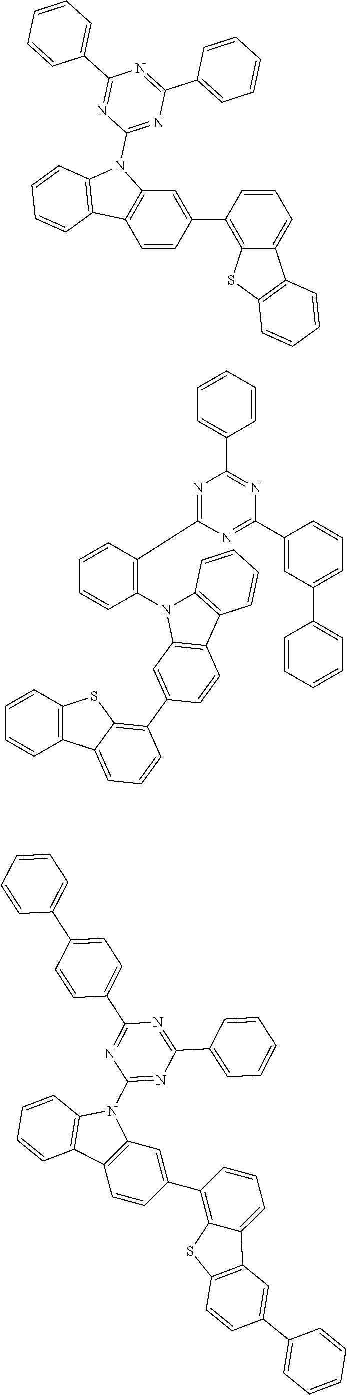 [SMILES: c1ccc(-c2nc(-c3ccccc3)nc(-n3c4ccccc4c4ccc(-c5cccc6c5sc5ccccc56)cc43)n2)cc1 ; c1ccc(-c2cccc(-c3nc(-c4ccccc4)nc(-c4ccccc4-n4c5ccccc5c5ccc(-c6cccc7c6sc6ccccc67)cc54)n3)c2)cc1 ; c1ccc(-c2ccc(-c3nc(-c4ccccc4)nc(-n4c5ccccc5c5ccc(-c6cccc7c6sc6ccc(-c8ccccc8)cc67)cc54)n3)cc2)cc1]

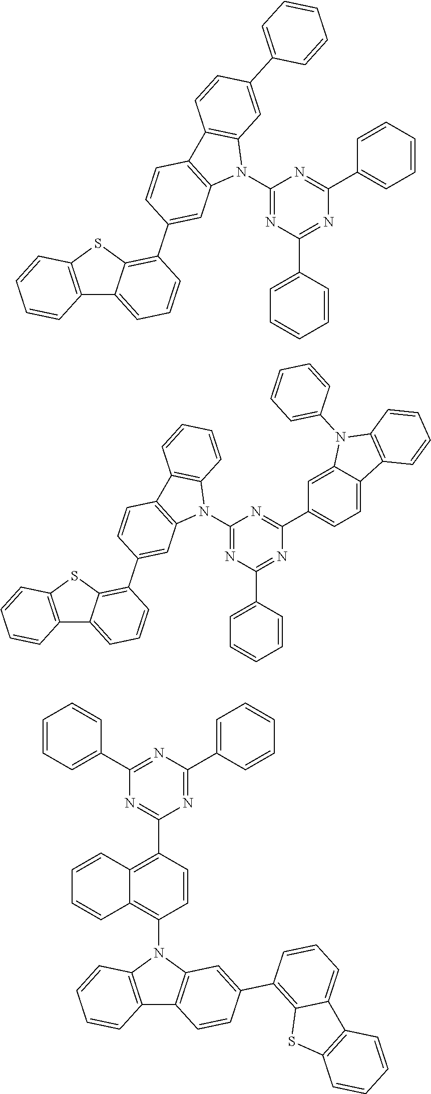

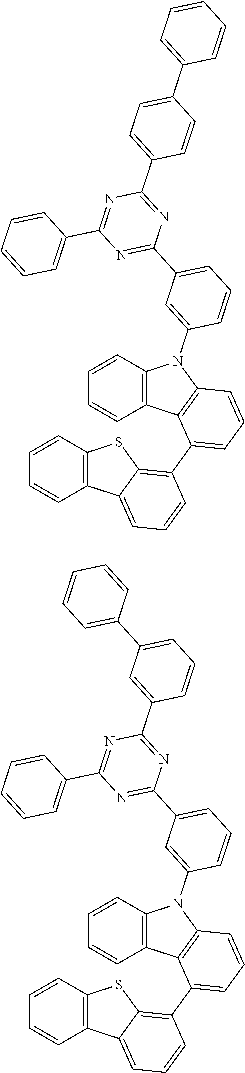 [SMILES: c1ccc(-c2ccc(-c3nc(-c4ccccc4)nc(-c4cccc(-n5c6ccccc6c6c(-c7cccc8c7sc7ccccc78)cccc65)c4)n3)cc2)cc1 ; c1ccc(-c2cccc(-c3nc(-c4ccccc4)nc(-c4cccc(-n5c6ccccc6c6c(-c7cccc8c7sc7ccccc78)cccc65)c4)n3)c2)cc1]

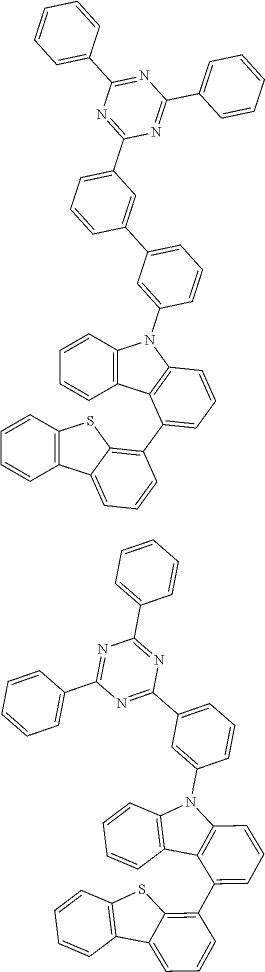

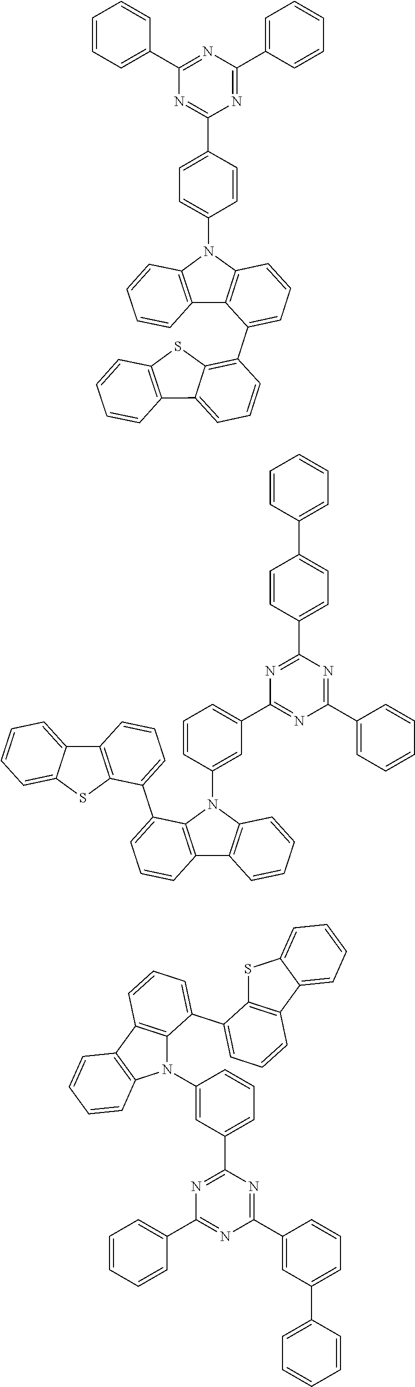 [SMILES: c1ccc(-c2nc(-c3ccccc3)nc(-c3ccc(-n4c5ccccc5c5c(-c6cccc7c6sc6ccccc67)cccc54)cc3)n2)cc1 ; c1ccc(-c2ccc(-c3nc(-c4ccccc4)nc(-c4cccc(-n5c6ccccc6c6cccc(-c7cccc8c7sc7ccccc78)c65)c4)n3)cc2)cc1 ; c1ccc(-c2cccc(-c3nc(-c4ccccc4)nc(-c4cccc(-n5c6ccccc6c6cccc(-c7cccc8c7sc7ccccc78)c65)c4)n3)c2)cc1]

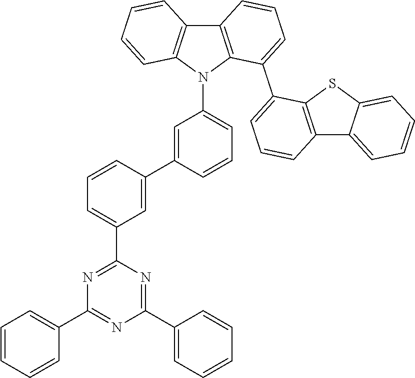 [SMILES: c1ccc(-c2nc(-c3ccccc3)nc(-c3cccc(-c4cccc(-n5c6ccccc6c6cccc(-c7cccc8c7sc7ccccc78)c65)c4)c3)n2)cc1]

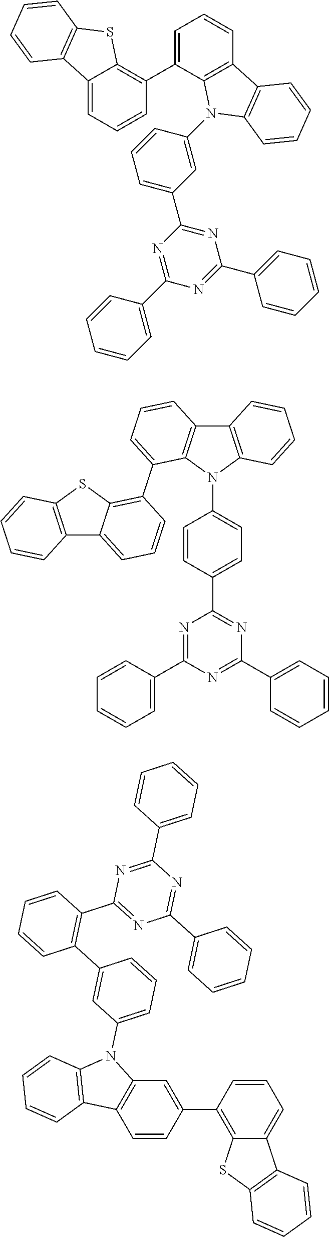 [SMILES: c1ccc(-c2nc(-c3ccccc3)nc(-c3cccc(-n4c5ccccc5c5cccc(-c6cccc7c6sc6ccccc67)c54)c3)n2)cc1 ; c1ccc(-c2nc(-c3ccccc3)nc(-c3ccc(-n4c5ccccc5c5cccc(-c6cccc7c6sc6ccccc67)c54)cc3)n2)cc1 ; c1ccc(-c2nc(-c3ccccc3)nc(-c3ccccc3-c3cccc(-n4c5ccccc5c5ccc(-c6cccc7c6sc6ccccc67)cc54)c3)n2)cc1]

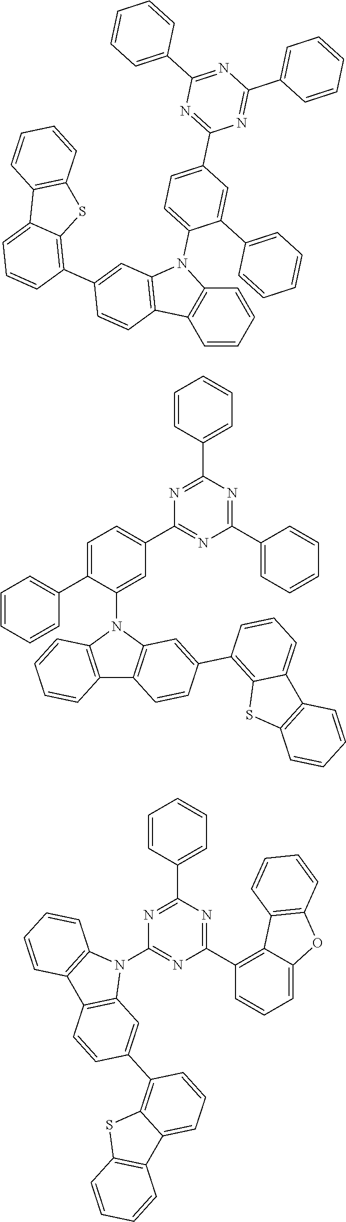

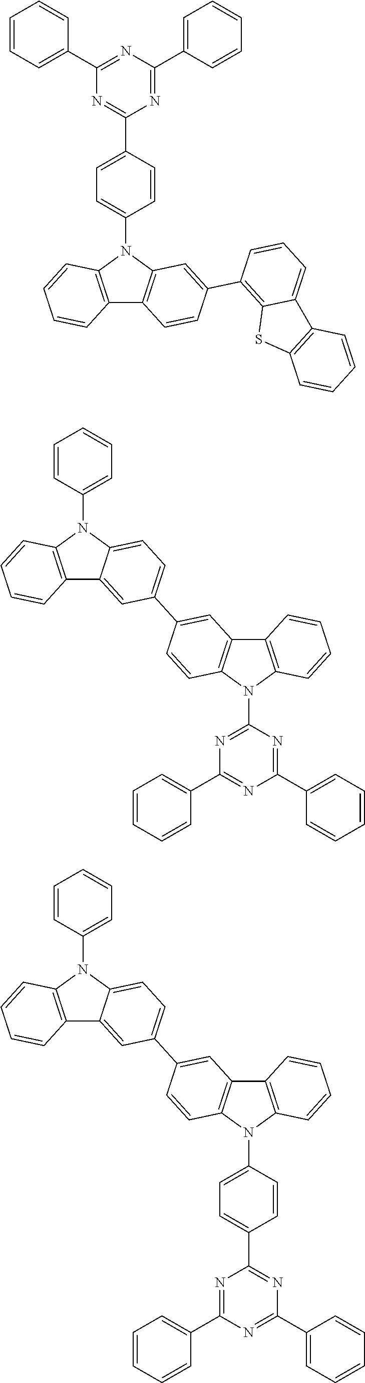 [SMILES: c1ccc(-c2nc(-c3ccccc3)nc(-c3ccc(-n4c5ccccc5c5ccc(-c6cccc7c6sc6ccccc67)cc54)cc3)n2)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

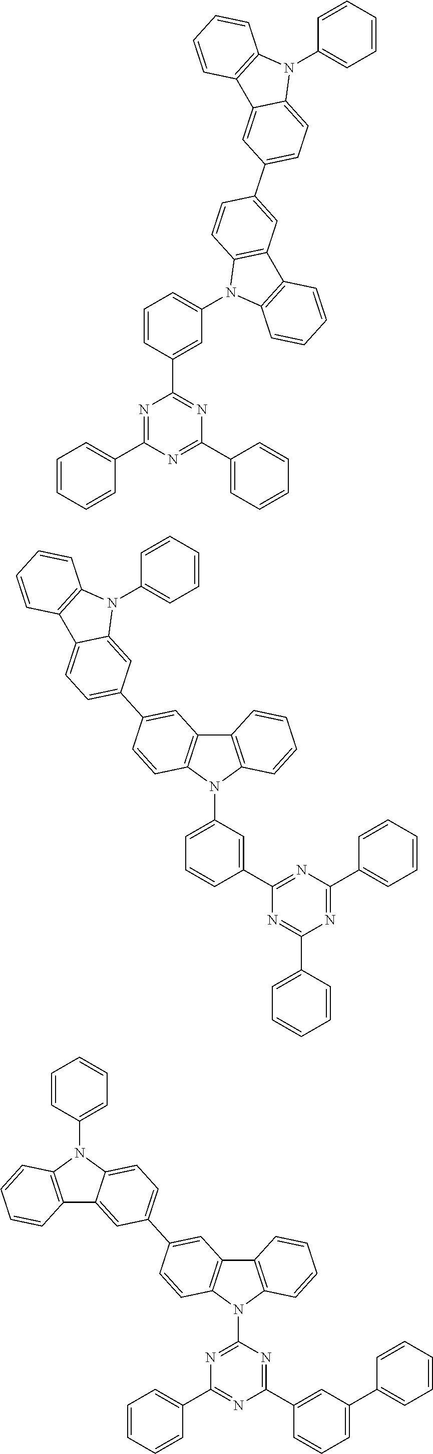 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2nc(-c3ccccc3)nc(-c3cccc(-n4c5ccccc5c5cc(-c6ccc7c8ccccc8n(-c8ccccc8)c7c6)ccc54)c3)n2)cc1 ; c1ccc(-c2cccc(-c3nc(-c4ccccc4)nc(-n4c5ccccc5c5cc(-c6ccc7c(c6)c6ccccc6n7-c6ccccc6)ccc54)n3)c2)cc1]

The compound represented by formula 12 may be at least one selected from the group consisting of the following compounds, but is not limited thereto.

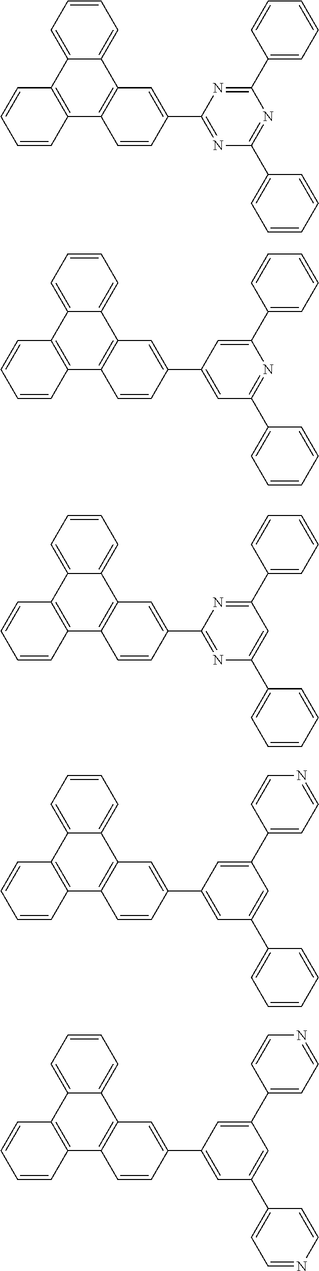

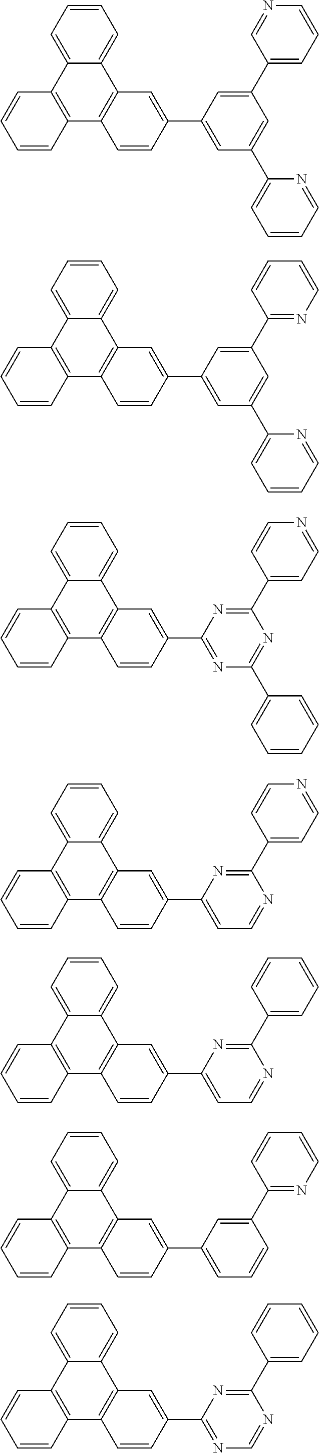

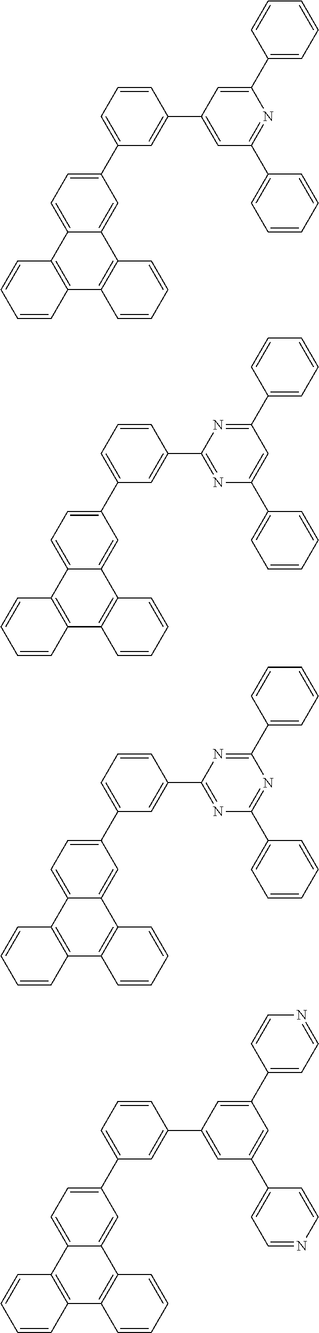 [SMILES: c1ccc(-c2cc(-c3cccc(-c4ccc5c6ccccc6c6ccccc6c5c4)c3)cc(-c3ccccc3)n2)cc1 ; c1ccc(-c2cc(-c3ccccc3)nc(-c3cccc(-c4ccc5c6ccccc6c6ccccc6c5c4)c3)n2)cc1 ; c1ccc(-c2nc(-c3ccccc3)nc(-c3cccc(-c4ccc5c6ccccc6c6ccccc6c5c4)c3)n2)cc1 ; c1cc(-c2cc(-c3ccncc3)cc(-c3ccncc3)c2)cc(-c2ccc3c4ccccc4c4ccccc4c3c2)c1]

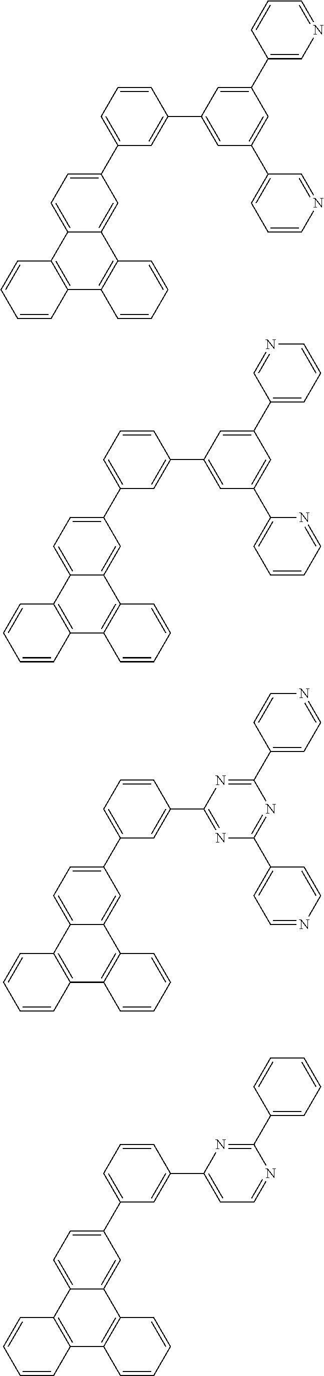 [SMILES: c1cncc(-c2cc(-c3cccnc3)cc(-c3cccc(-c4ccc5c6ccccc6c6ccccc6c5c4)c3)c2)c1 ; c1ccc(-c2cc(-c3cccnc3)cc(-c3cccc(-c4ccc5c6ccccc6c6ccccc6c5c4)c3)c2)nc1 ; c1cc(-c2ccc3c4ccccc4c4ccccc4c3c2)cc(-c2nc(-c3ccncc3)nc(-c3ccncc3)n2)c1 ; c1ccc(-c2nccc(-c3cccc(-c4ccc5c6ccccc6c6ccccc6c5c4)c3)n2)cc1]

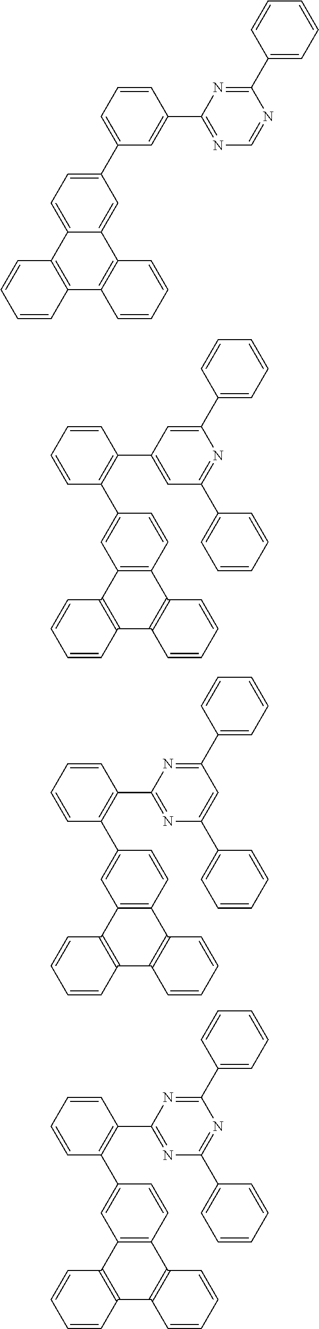

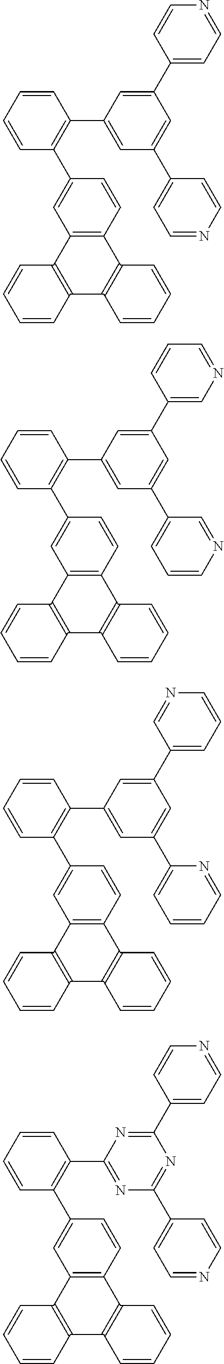

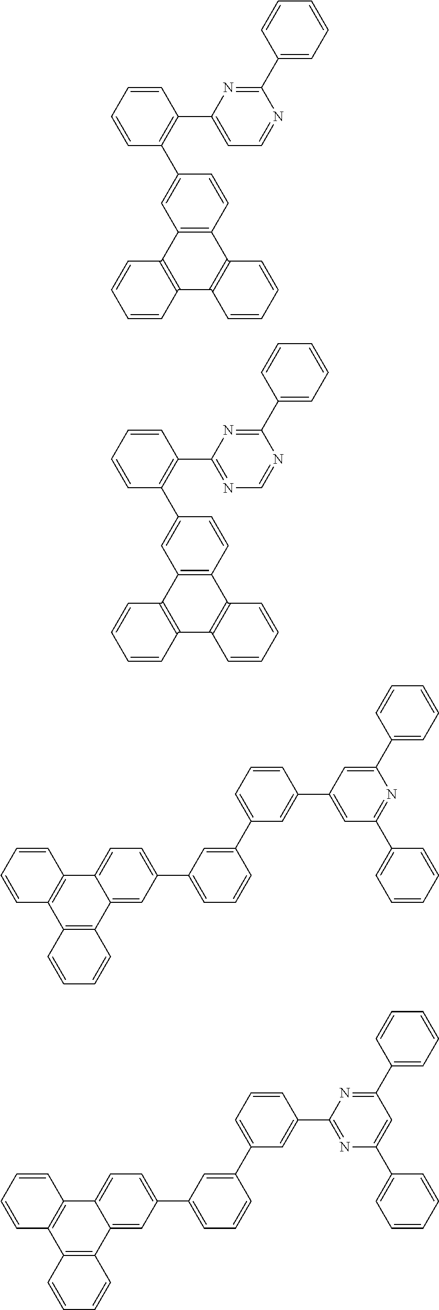

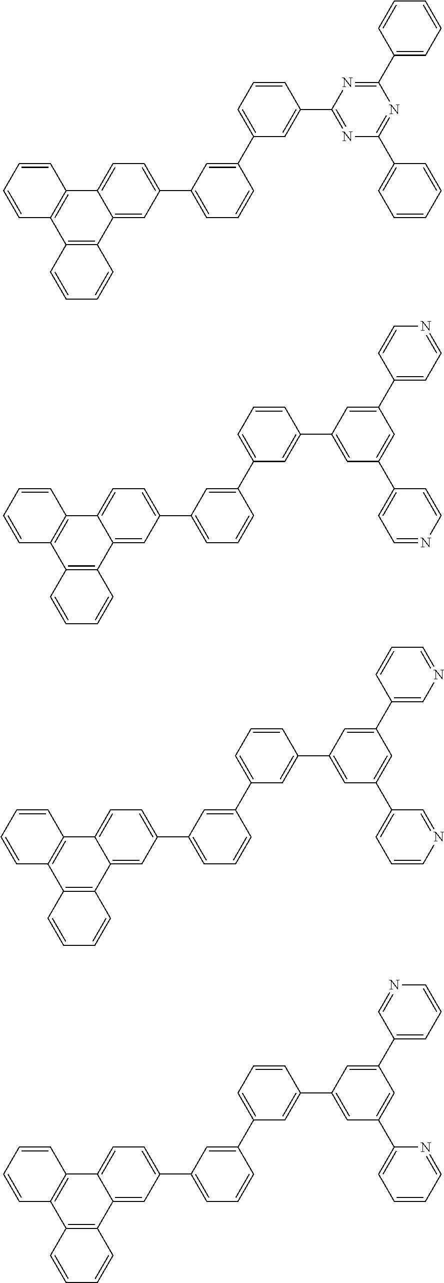 [SMILES: c1ccc(-c2nc(-c3ccccc3)nc(-c3cccc(-c4cccc(-c5ccc6c7ccccc7c7ccccc7c6c5)c4)c3)n2)cc1 ; c1cc(-c2cccc(-c3ccc4c5ccccc5c5ccccc5c4c3)c2)cc(-c2cc(-c3ccncc3)cc(-c3ccncc3)c2)c1 ; c1cncc(-c2cc(-c3cccnc3)cc(-c3cccc(-c4cccc(-c5ccc6c7ccccc7c7ccccc7c6c5)c4)c3)c2)c1 ; c1ccc(-c2cc(-c3cccnc3)cc(-c3cccc(-c4cccc(-c5ccc6c7ccccc7c7ccccc7c6c5)c4)c3)c2)nc1]

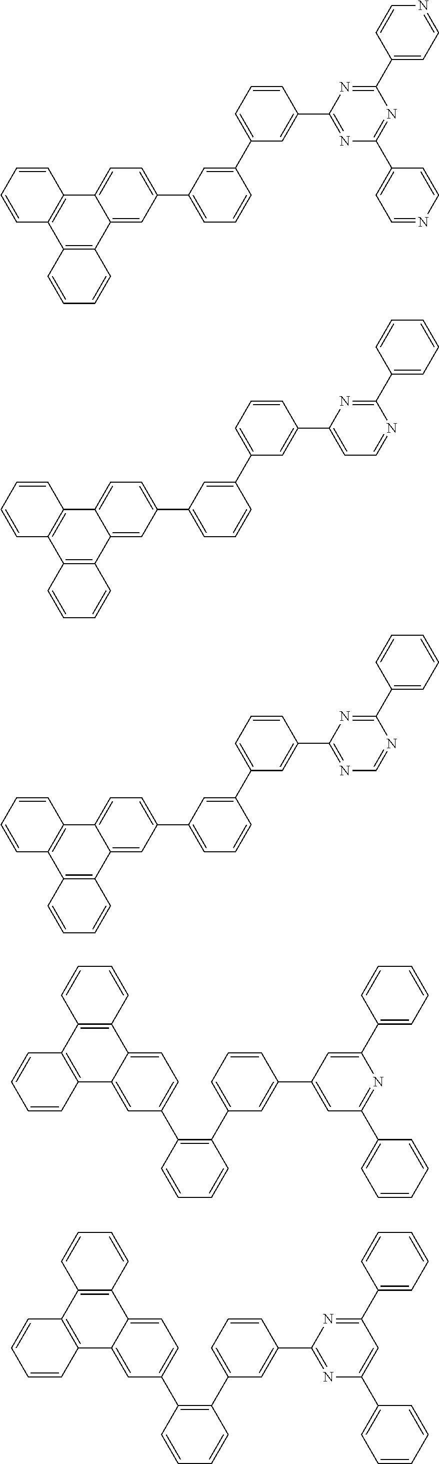

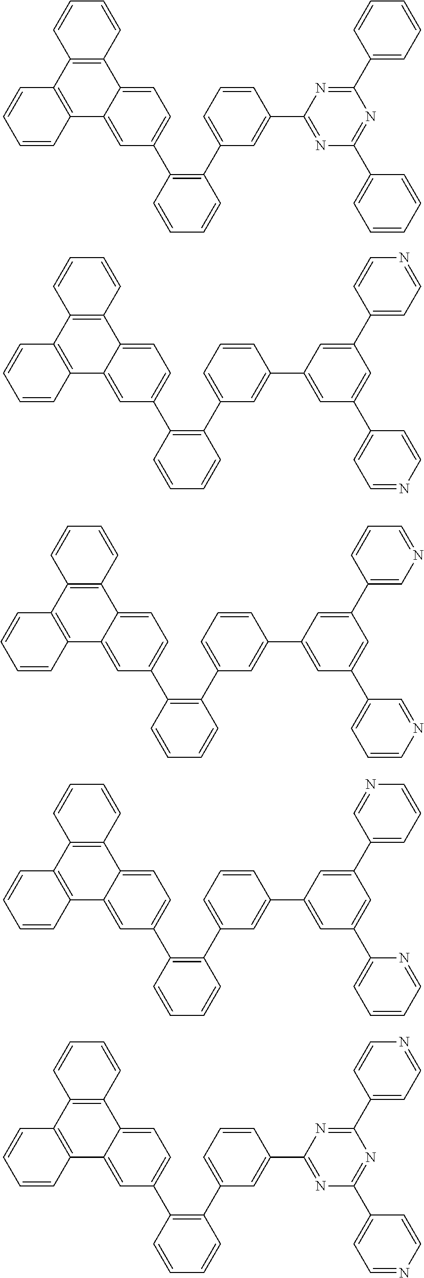

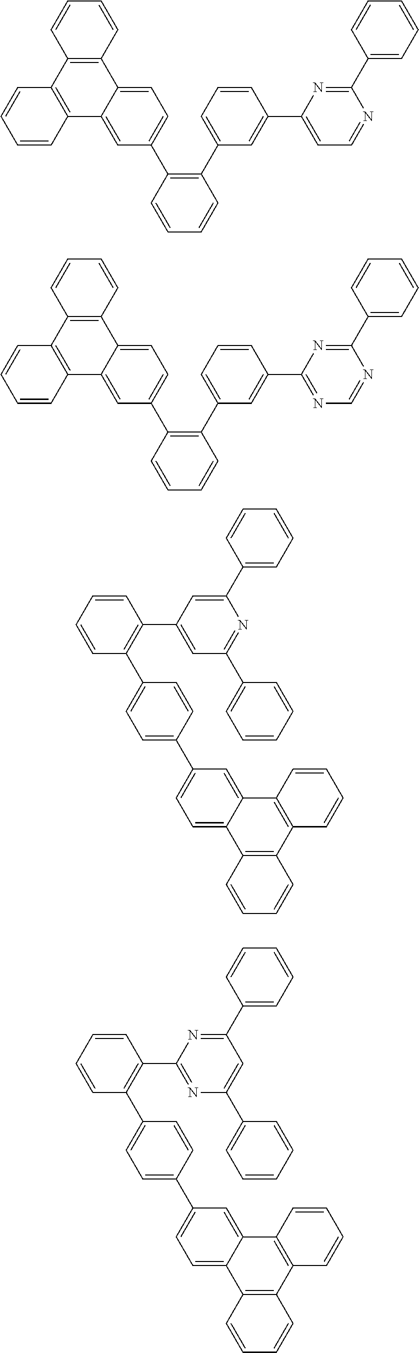

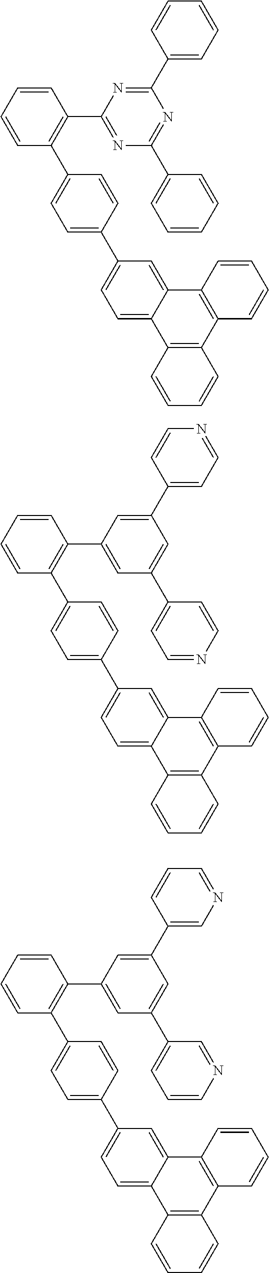

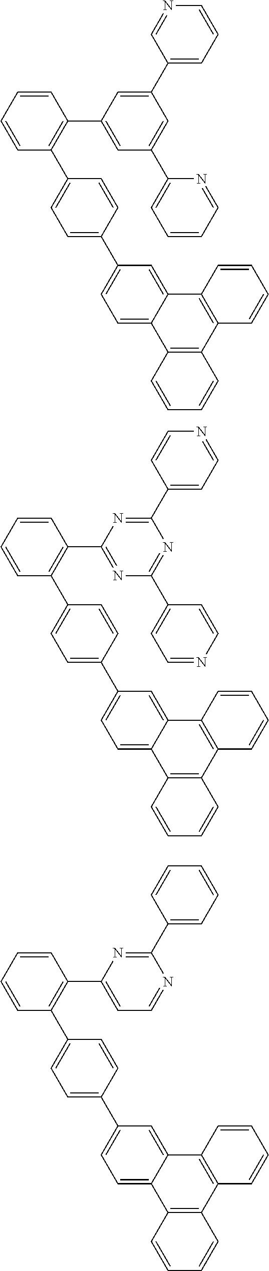

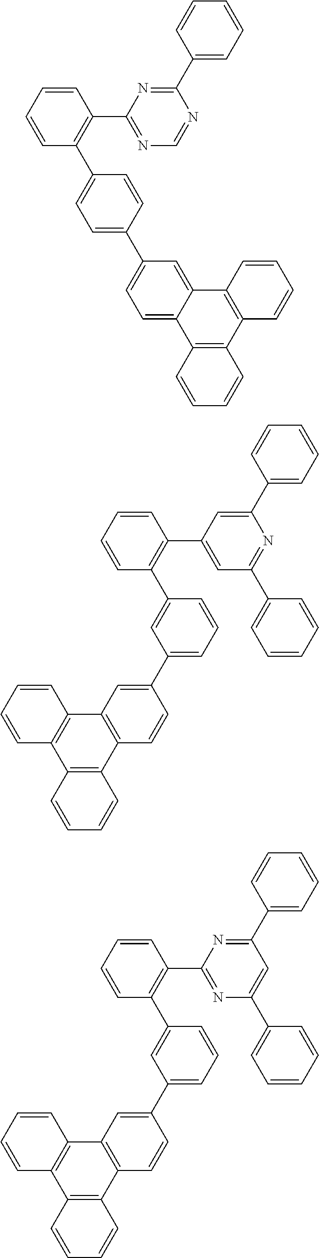

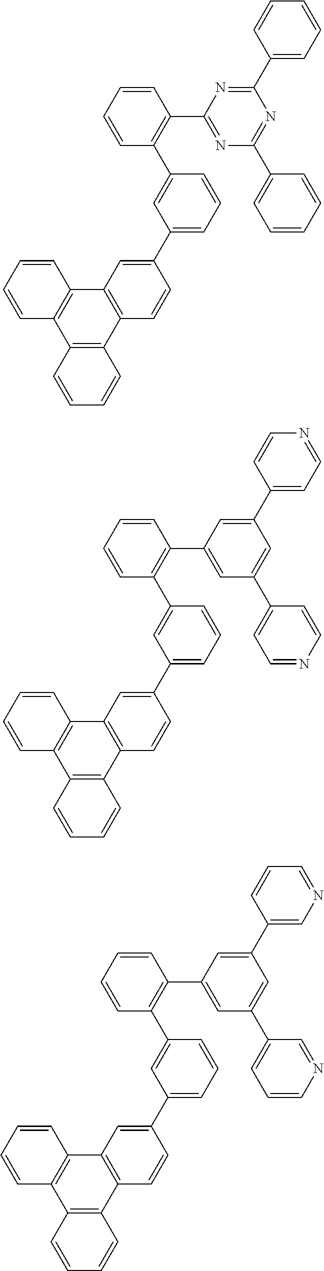 [SMILES: c1ccc(-c2nc(-c3ccccc3)nc(-c3ccccc3-c3cccc(-c4ccc5c6ccccc6c6ccccc6c5c4)c3)n2)cc1 ; c1cc(-c2ccc3c4ccccc4c4ccccc4c3c2)cc(-c2ccccc2-c2cc(-c3ccncc3)cc(-c3ccncc3)c2)c1 ; c1cncc(-c2cc(-c3cccnc3)cc(-c3ccccc3-c3cccc(-c4ccc5c6ccccc6c6ccccc6c5c4)c3)c2)c1]

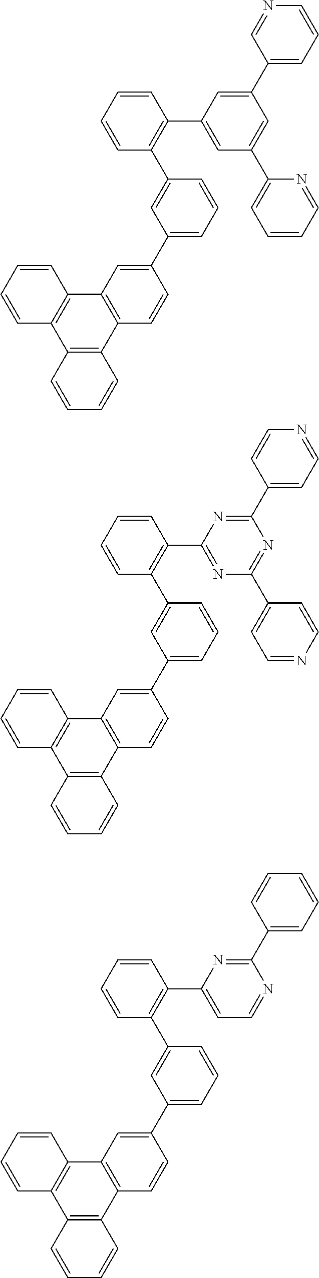 [SMILES: c1ccc(-c2cc(-c3cccnc3)cc(-c3ccccc3-c3cccc(-c4ccc5c6ccccc6c6ccccc6c5c4)c3)c2)nc1 ; c1cc(-c2ccc3c4ccccc4c4ccccc4c3c2)cc(-c2ccccc2-c2nc(-c3ccncc3)nc(-c3ccncc3)n2)c1 ; c1ccc(-c2nccc(-c3ccccc3-c3cccc(-c4ccc5c6ccccc6c6ccccc6c5c4)c3)n2)cc1]

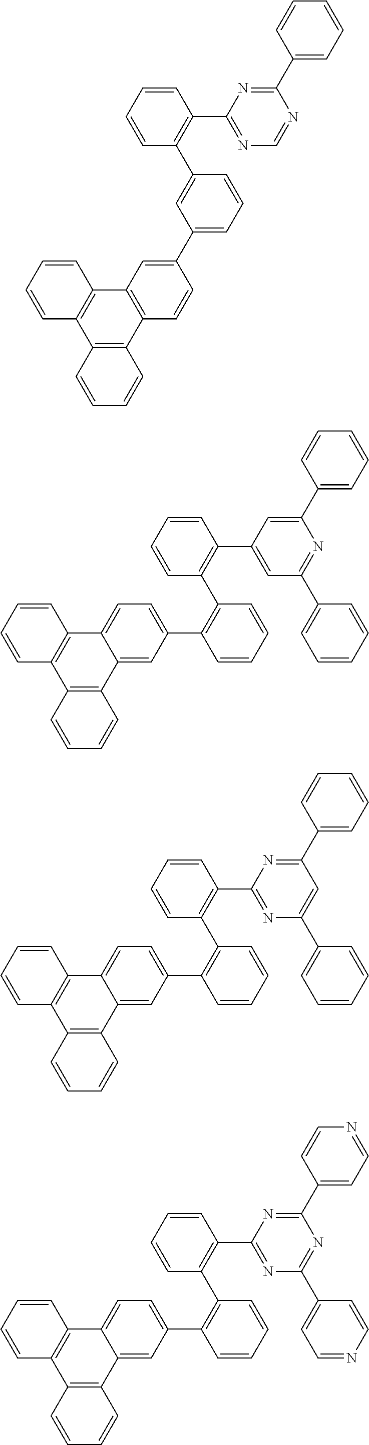 [SMILES: c1ccc(-c2ncnc(-c3ccccc3-c3cccc(-c4ccc5c6ccccc6c6ccccc6c5c4)c3)n2)cc1 ; c1ccc(-c2cc(-c3ccccc3-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccccc3-c3ccc4c5ccccc5c5ccccc5c4c3)n2)cc1 ; c1ccc(-c2ccccc2-c2nc(-c3ccncc3)nc(-c3ccncc3)n2)c(-c2ccc3c4ccccc4c4ccccc4c3c2)c1]

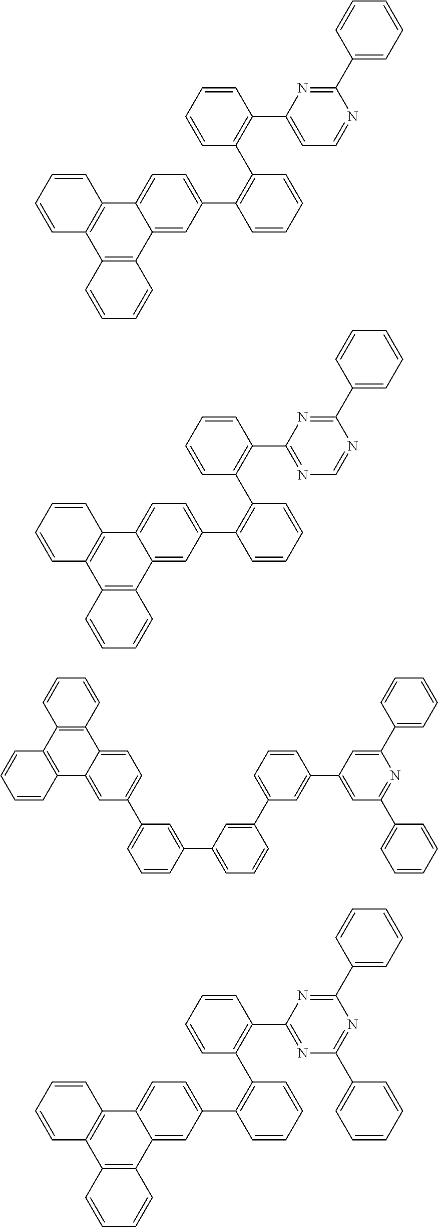

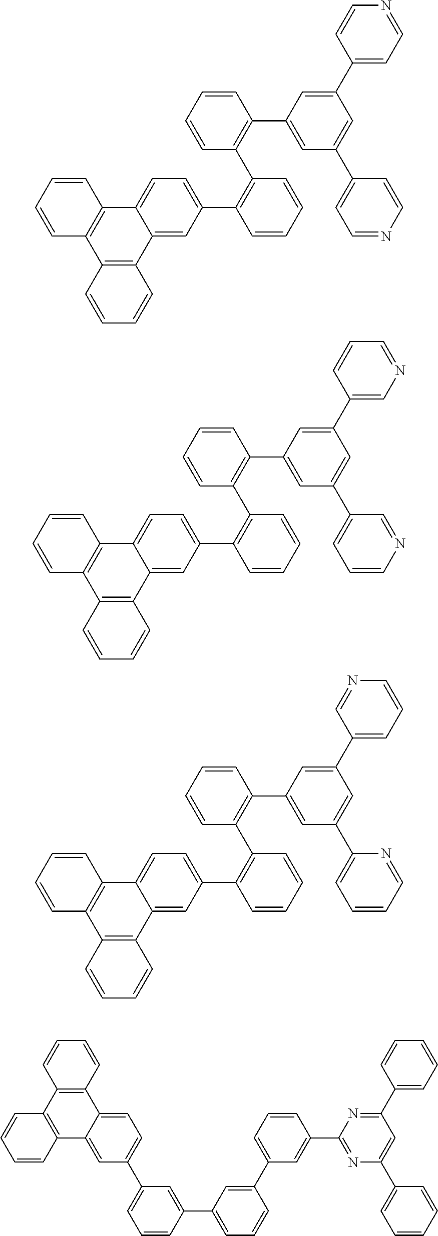

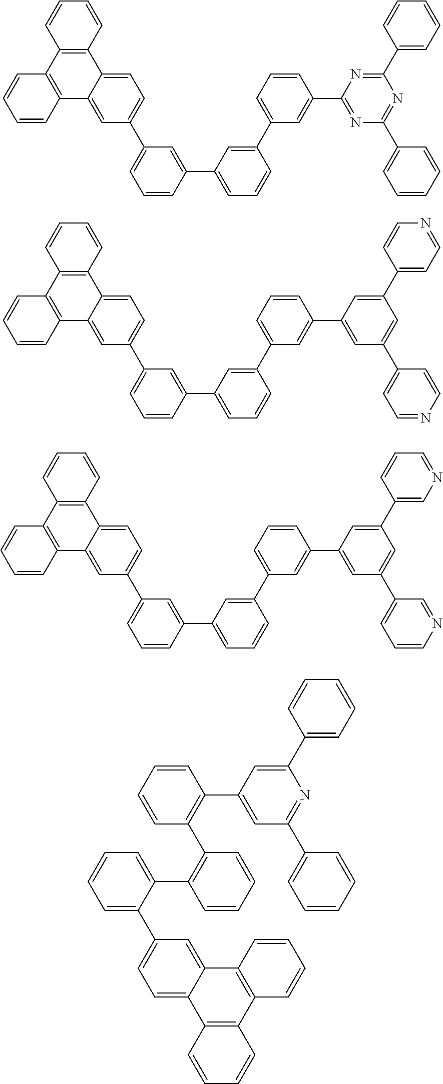

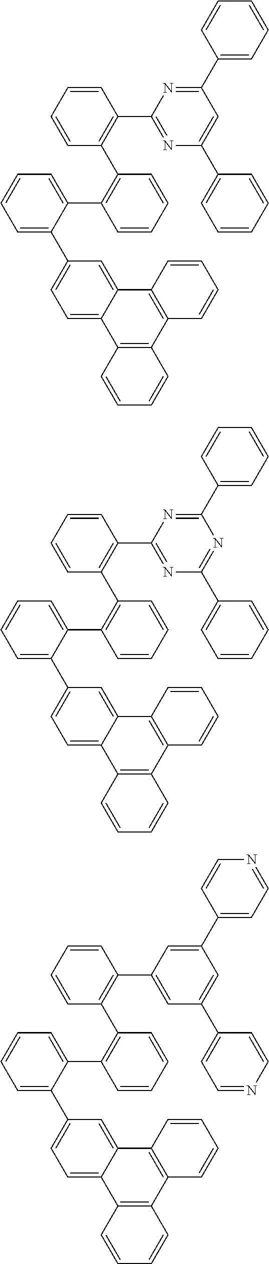 [SMILES: c1ccc(-c2cc(-c3ccccc3)nc(-c3ccccc3-c3ccccc3-c3ccccc3-c3ccc4c5ccccc5c5ccccc5c4c3)n2)cc1 ; c1ccc(-c2nc(-c3ccccc3)nc(-c3ccccc3-c3ccccc3-c3ccccc3-c3ccc4c5ccccc5c5ccccc5c4c3)n2)cc1 ; c1ccc(-c2ccccc2-c2ccccc2-c2ccc3c4ccccc4c4ccccc4c3c2)c(-c2cc(-c3ccncc3)cc(-c3ccncc3)c2)c1]

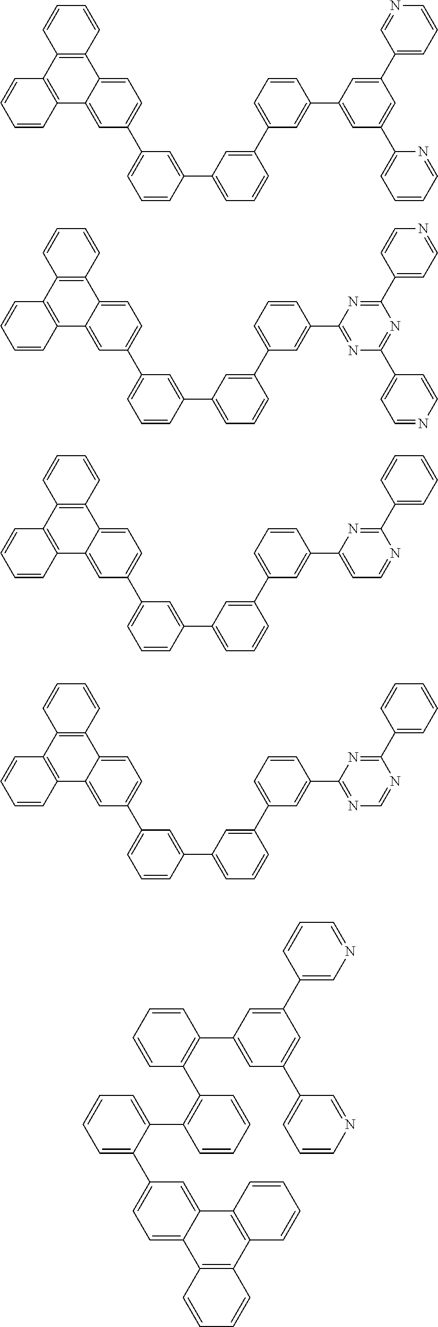

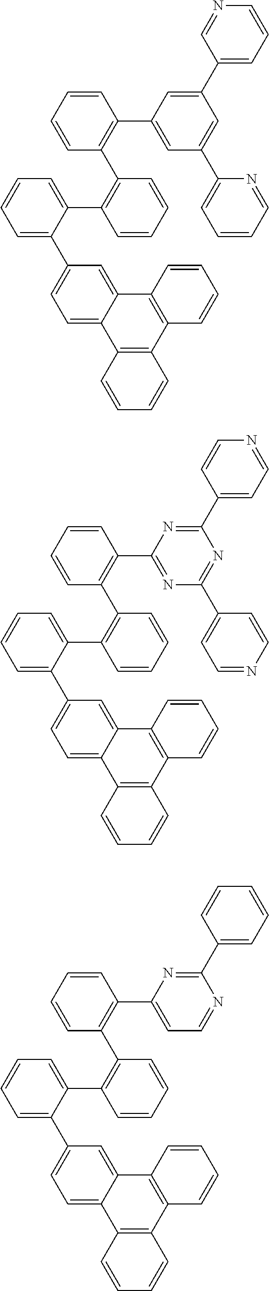 [SMILES: c1ccc(-c2cc(-c3cccnc3)cc(-c3ccccc3-c3ccccc3-c3ccccc3-c3ccc4c5ccccc5c5ccccc5c4c3)c2)nc1 ; c1ccc(-c2ccccc2-c2ccccc2-c2nc(-c3ccncc3)nc(-c3ccncc3)n2)c(-c2ccc3c4ccccc4c4ccccc4c3c2)c1 ; c1ccc(-c2nccc(-c3ccccc3-c3ccccc3-c3ccccc3-c3ccc4c5ccccc5c5ccccc5c4c3)n2)cc1]

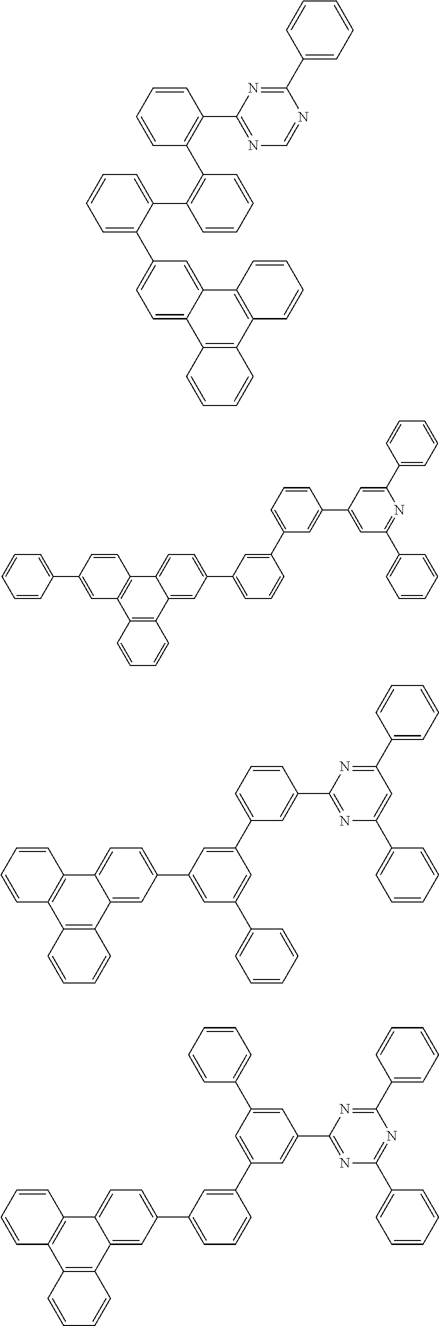 [SMILES: c1ccc(-c2ncnc(-c3ccccc3-c3ccccc3-c3ccccc3-c3ccc4c5ccccc5c5ccccc5c4c3)n2)cc1 ; c1ccc(-c2ccc3c4ccc(-c5cccc(-c6cccc(-c7cc(-c8ccccc8)nc(-c8ccccc8)c7)c6)c5)cc4c4ccccc4c3c2)cc1 ; c1ccc(-c2cc(-c3cccc(-c4nc(-c5ccccc5)cc(-c5ccccc5)n4)c3)cc(-c3ccc4c5ccccc5c5ccccc5c4c3)c2)cc1 ; c1ccc(-c2cc(-c3cccc(-c4ccc5c6ccccc6c6ccccc6c5c4)c3)cc(-c3nc(-c4ccccc4)nc(-c4ccccc4)n3)c2)cc1]

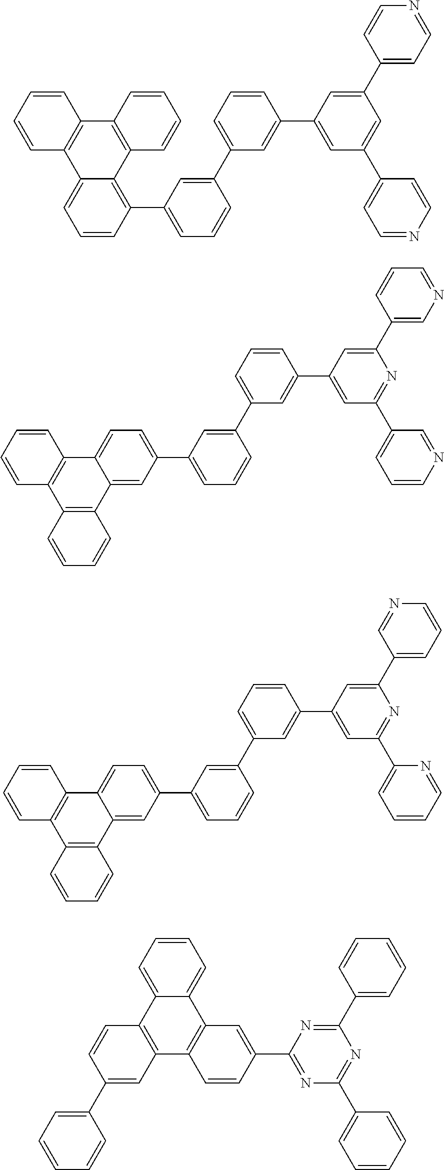

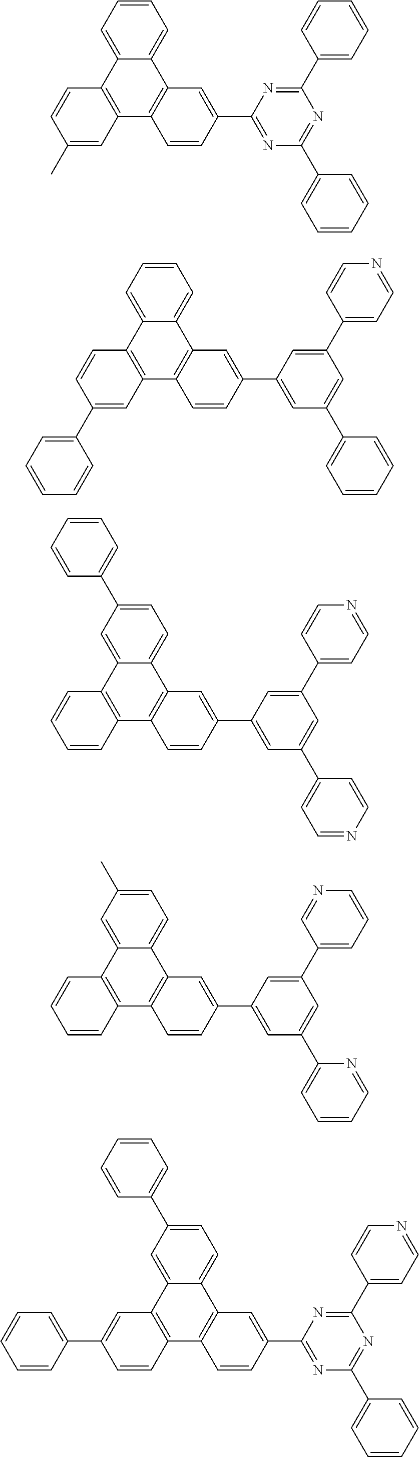

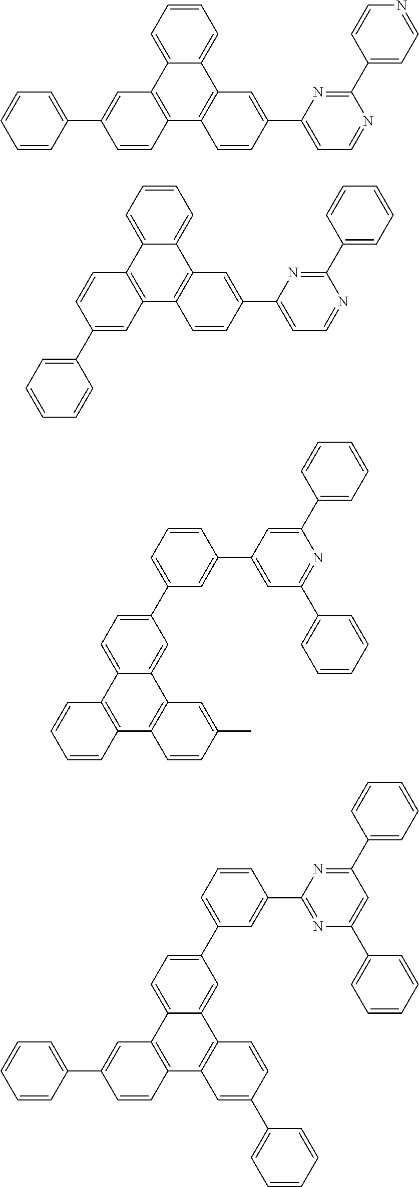

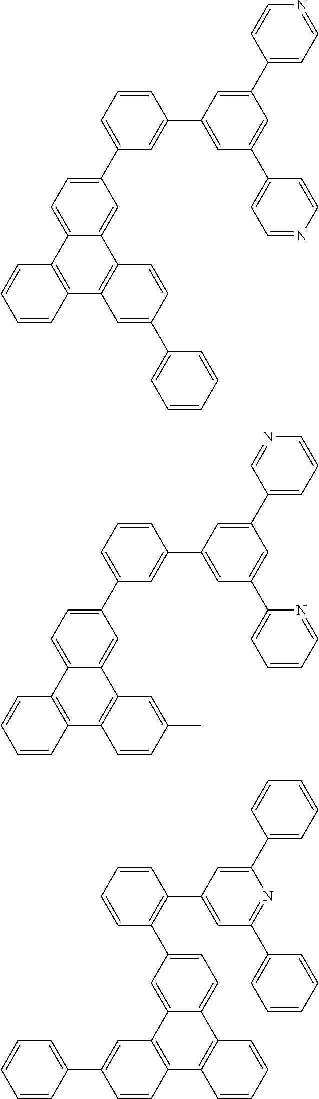 [SMILES: c1ccc(-c2ccc3c(c2)c2ccccc2c2ccc(-c4cccc(-c5cc(-c6ccncc6)cc(-c6ccncc6)c5)c4)cc23)cc1 ; Cc1ccc2c3ccccc3c3ccc(-c4cccc(-c5cc(-c6cccnc6)cc(-c6ccccn6)c5)c4)cc3c2c1 ; c1ccc(-c2ccc3c4ccccc4c4ccc(-c5ccccc5-c5cc(-c6ccccc6)nc(-c6ccccc6)c5)cc4c3c2)cc1]

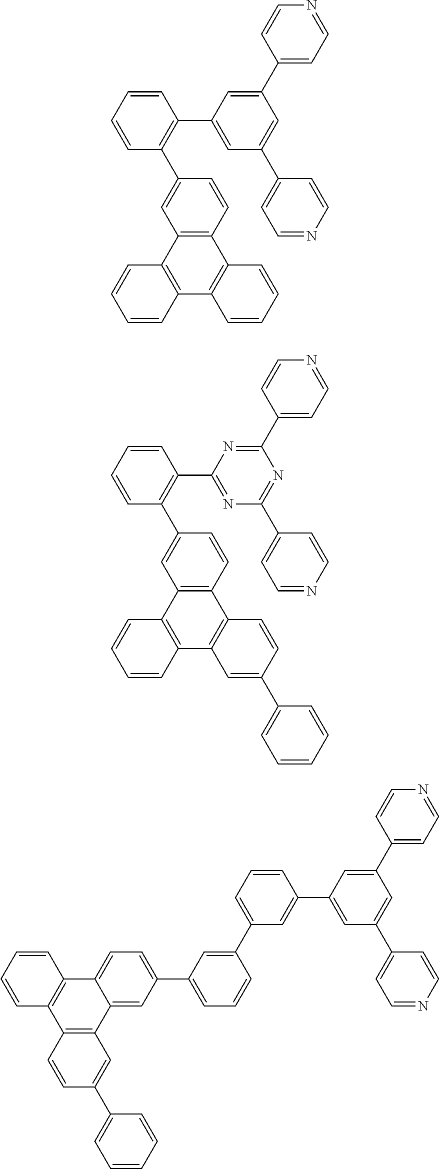

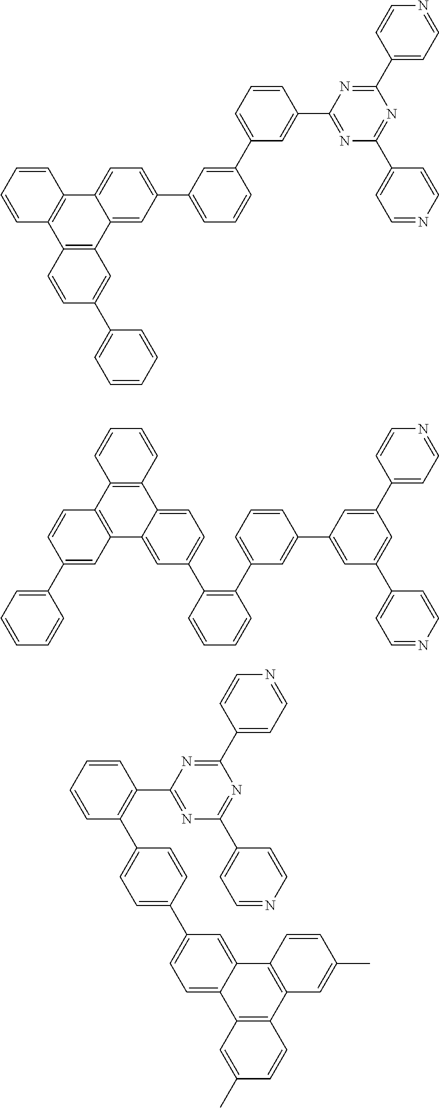

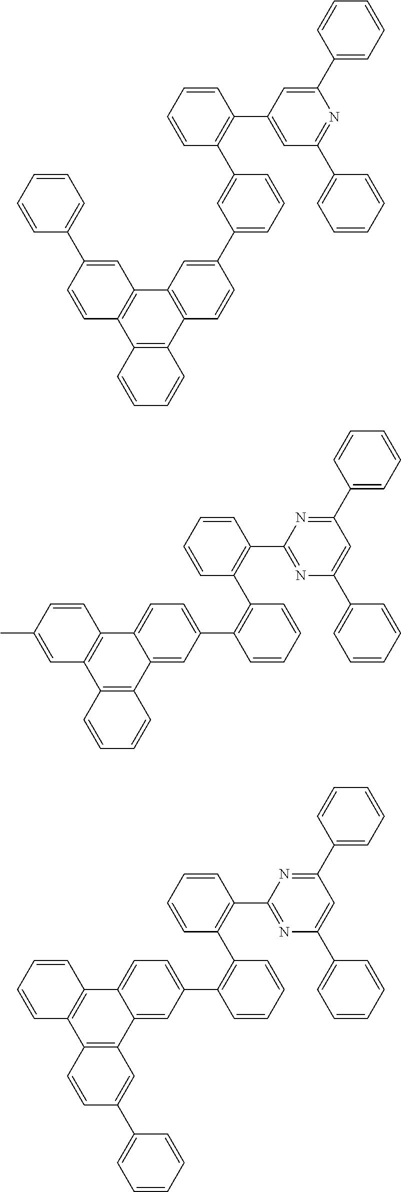 [SMILES: c1ccc(-c2ccc3c4ccccc4c4ccc(-c5cccc(-c6ccccc6-c6cc(-c7ccccc7)nc(-c7ccccc7)c6)c5)cc4c3c2)cc1 ; Cc1ccc2c3ccc(-c4ccccc4-c4ccccc4-c4nc(-c5ccccc5)cc(-c5ccccc5)n4)cc3c3ccccc3c2c1 ; c1ccc(-c2ccc3c4ccccc4c4ccc(-c5ccccc5-c5ccccc5-c5nc(-c6ccccc6)cc(-c6ccccc6)n5)cc4c3c2)cc1]

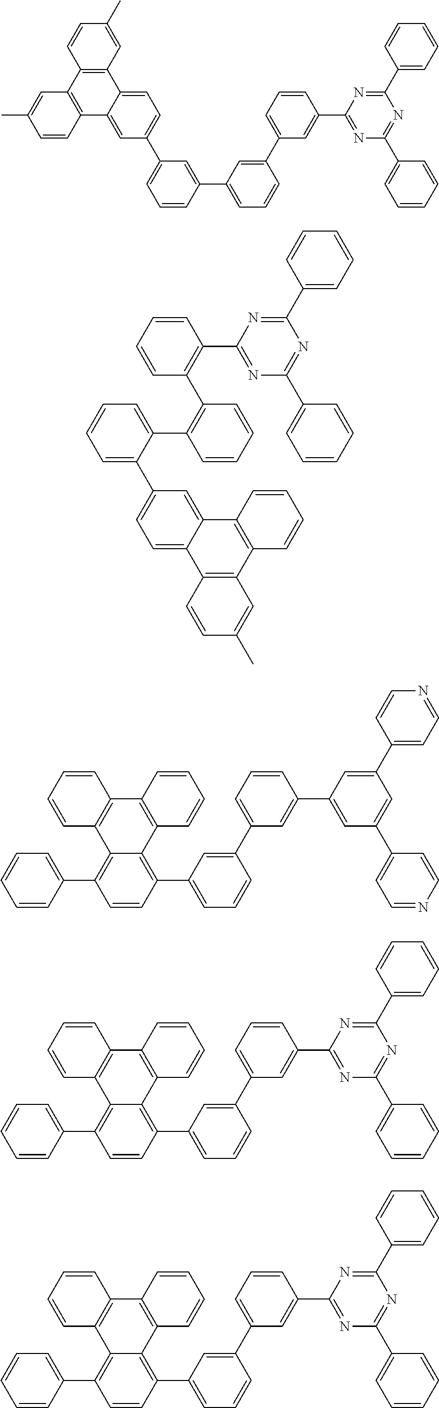

The compound represented by formula 13 or 14 may be at least one selected from the group consisting of the following compounds, but is not limited thereto.

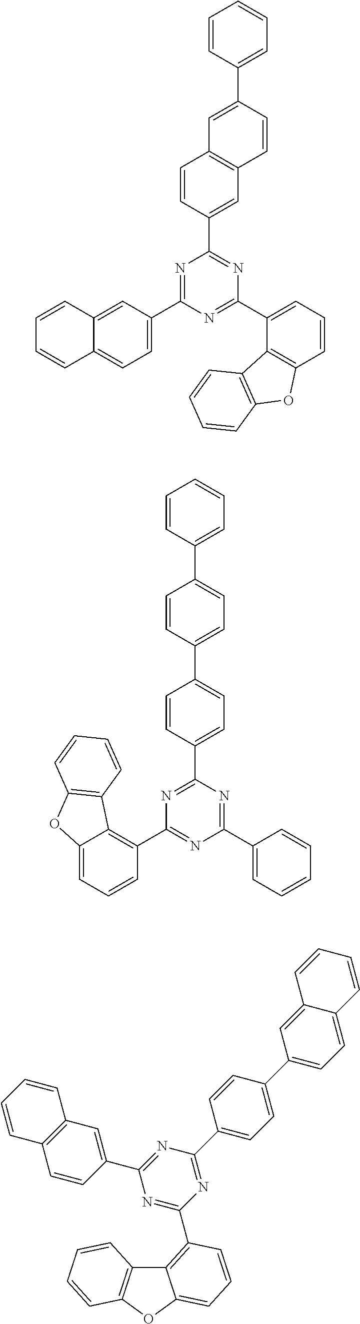 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccccc5)nc(-c5cccc6oc7ccccc7c56)n4)cc3)cc2)cc1 ; c1ccc2cc(-c3ccc(-c4nc(-c5ccc6ccccc6c5)nc(-c5cccc6oc7ccccc7c56)n4)cc3)ccc2c1]

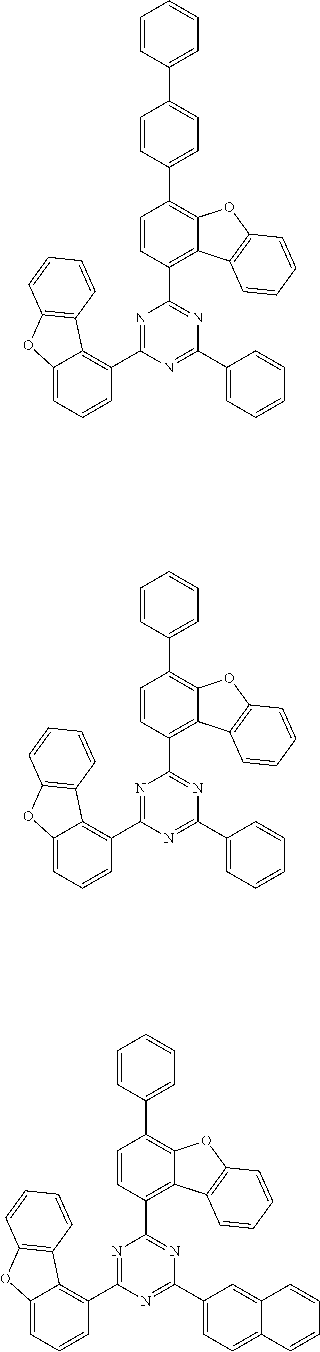 [SMILES: c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2nc(-c3cccc4oc5ccccc5c34)nc(-c3ccc(-c4ccccc4)c4oc5ccccc5c34)n2)cc1 ; c1ccc(-c2ccc(-c3nc(-c4ccc5ccccc5c4)nc(-c4cccc5oc6ccccc6c45)n3)c3c2oc2ccccc23)cc1]

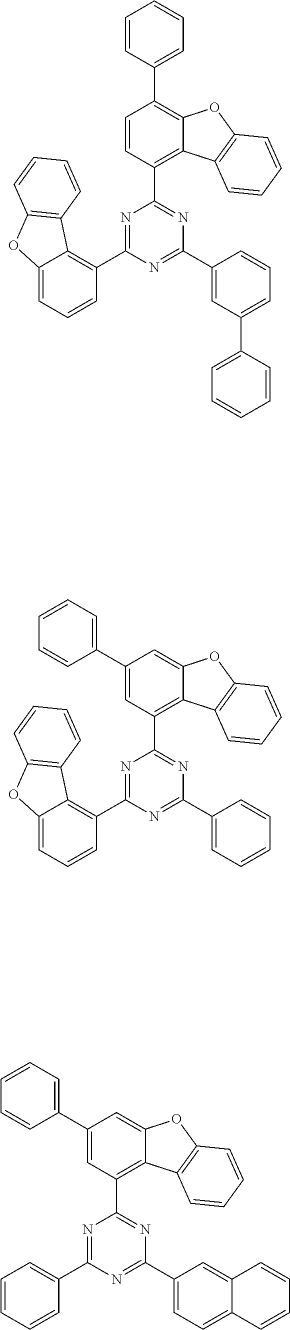 [SMILES: c1ccc(-c2cccc(-c3nc(-c4cccc5oc6ccccc6c45)nc(-c4ccc(-c5ccccc5)c5oc6ccccc6c45)n3)c2)cc1 ; c1ccc(-c2cc(-c3nc(-c4ccccc4)nc(-c4cccc5oc6ccccc6c45)n3)c3c(c2)oc2ccccc23)cc1 ; c1ccc(-c2cc(-c3nc(-c4ccccc4)nc(-c4ccc5ccccc5c4)n3)c3c(c2)oc2ccccc23)cc1]

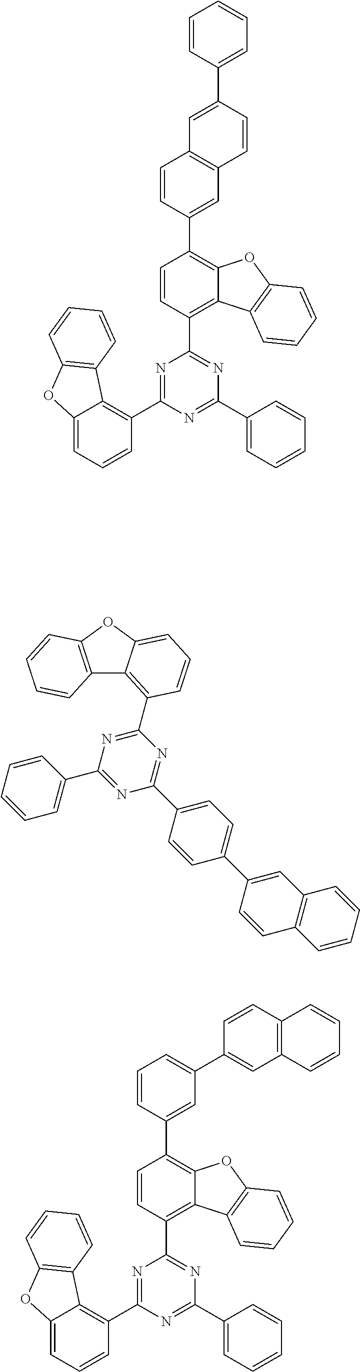 [SMILES: c1ccc(-c2ccc3cc(-c4ccc(-c5nc(-c6ccccc6)nc(-c6cccc7oc8ccccc8c67)n5)c5c4oc4ccccc45)ccc3c2)cc1 ; c1ccc(-c2nc(-c3ccc(-c4ccc5ccccc5c4)cc3)nc(-c3cccc4oc5ccccc5c34)n2)cc1 ; c1ccc(-c2nc(-c3cccc4oc5ccccc5c34)nc(-c3ccc(-c4cccc(-c5ccc6ccccc6c5)c4)c4oc5ccccc5c34)n2)cc1]

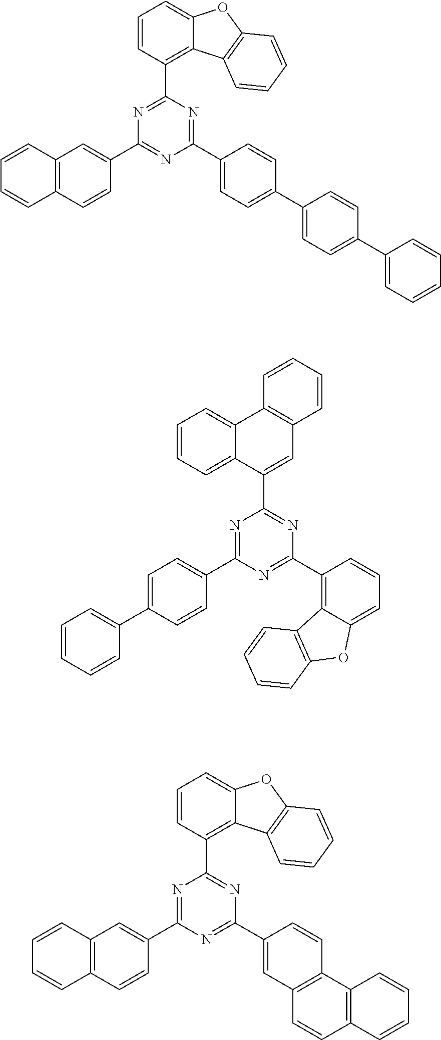 [SMILES: c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3nc(-c4cc5ccccc5c5ccccc45)nc(-c4cccc5oc6ccccc6c45)n3)cc2)cc1 ; c1ccc2cc(-c3nc(-c4ccc5c(ccc6ccccc65)c4)nc(-c4cccc5oc6ccccc6c45)n3)ccc2c1]

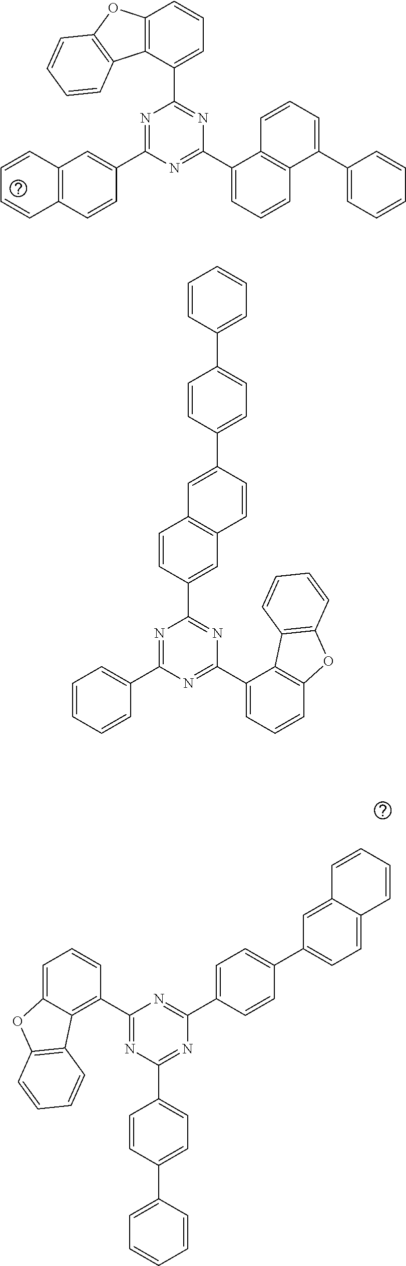 [SMILES: c1ccc(-c2cccc3c(-c4nc(-c5ccc6ccccc6c5)nc(-c5cccc6oc7ccccc7c56)n4)cccc23)cc1 ; c1ccc(-c2ccc(-c3ccc4cc(-c5nc(-c6ccccc6)nc(-c6cccc7oc8ccccc8c67)n5)ccc4c3)cc2)cc1 ; c1ccc(-c2ccc(-c3nc(-c4ccc(-c5ccc6ccccc6c5)cc4)nc(-c4cccc5oc6ccccc6c45)n3)cc2)cc1]

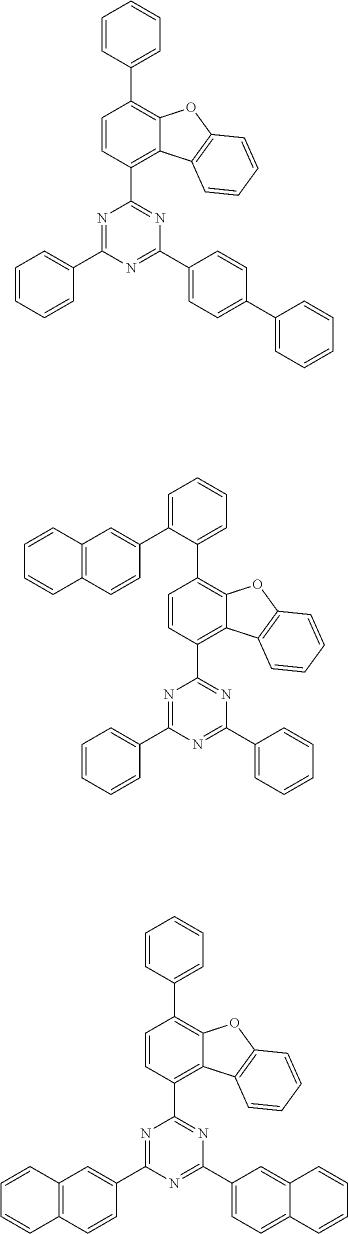 [SMILES: c1ccc(-c2ccc(-c3nc(-c4ccccc4)nc(-c4ccc(-c5ccccc5)c5oc6ccccc6c45)n3)cc2)cc1 ; c1ccc(-c2nc(-c3ccccc3)nc(-c3ccc(-c4ccccc4-c4ccc5ccccc5c4)c4oc5ccccc5c34)n2)cc1 ; c1ccc(-c2ccc(-c3nc(-c4ccc5ccccc5c4)nc(-c4ccc5ccccc5c4)n3)c3c2oc2ccccc23)cc1]

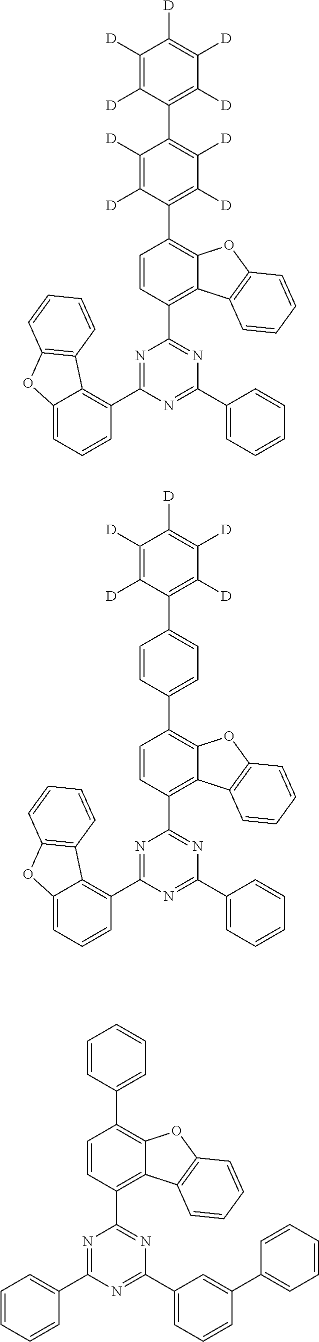 [SMILES: [2H]c1c([2H])c([2H])c(-c2c([2H])c([2H])c(-c3ccc(-c4nc(-c5ccccc5)nc(-c5cccc6oc7ccccc7c56)n4)c4c3oc3ccccc34)c([2H])c2[2H])c([2H])c1[2H] ; [2H]c1c([2H])c([2H])c(-c2ccc(-c3ccc(-c4nc(-c5ccccc5)nc(-c5cccc6oc7ccccc7c56)n4)c4c3oc3ccccc34)cc2)c([2H])c1[2H] ; c1ccc(-c2cccc(-c3nc(-c4ccccc4)nc(-c4ccc(-c5ccccc5)c5oc6ccccc6c45)n3)c2)cc1]

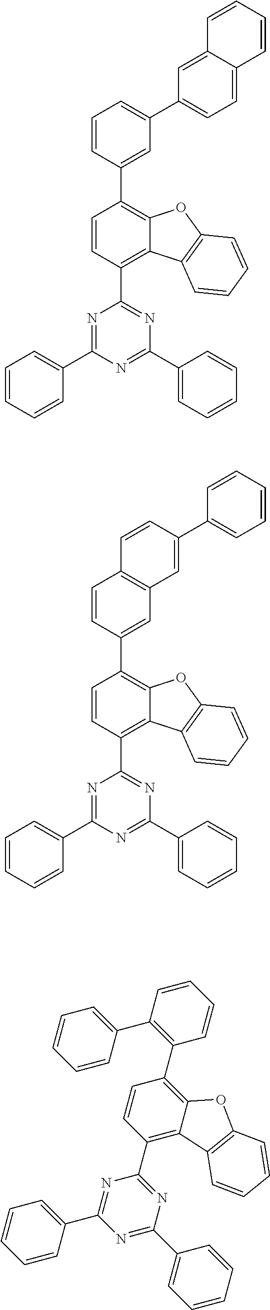 [SMILES: c1ccc(-c2nc(-c3ccccc3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccccc6)n5)c5c4oc4ccccc45)cc3c2)cc1 ; c1ccc(-c2nc(-c3ccccc3)nc(-c3ccc(-c4ccccc4-c4ccccc4)c4oc5ccccc5c34)n2)cc1]

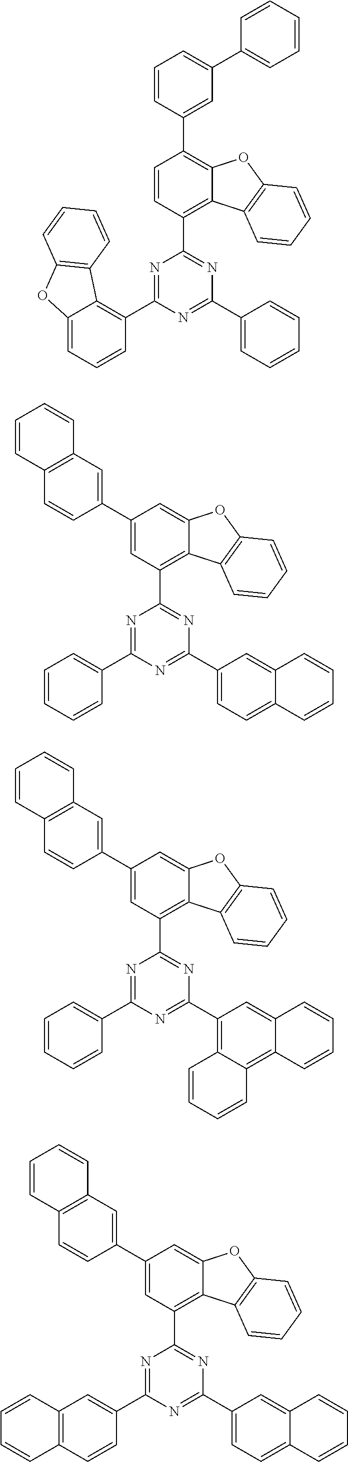

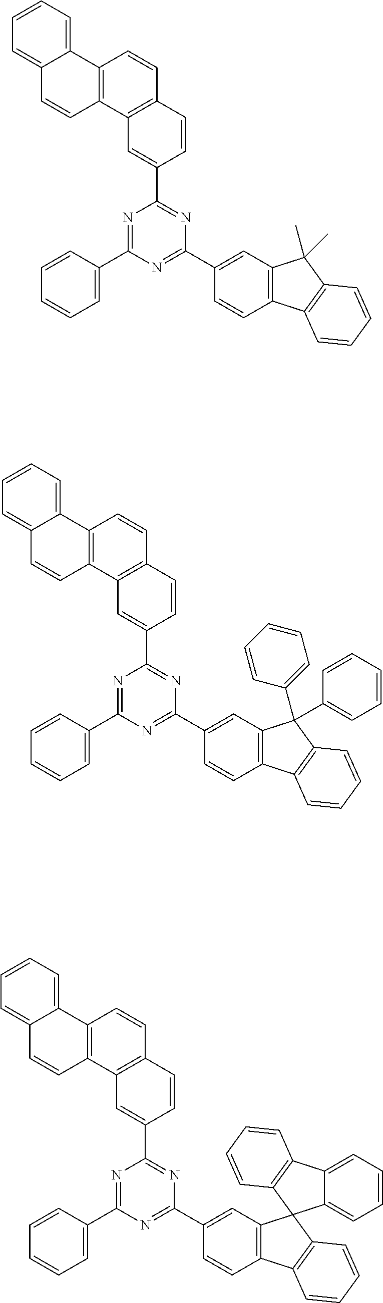 [SMILES: CC1(C)c2ccccc2-c2ccc(-c3nc(-c4ccccc4)nc(-c4ccc5ccc6c7ccccc7ccc6c5c4)n3)cc21 ; c1ccc(-c2nc(-c3ccc4c(c3)C(c3ccccc3)(c3ccccc3)c3ccccc3-4)nc(-c3ccc4ccc5c6ccccc6ccc5c4c3)n2)cc1 ; c1ccc(-c2nc(-c3ccc4c(c3)C3(c5ccccc5-c5ccccc53)c3ccccc3-4)nc(-c3ccc4ccc5c6ccccc6ccc5c4c3)n2)cc1]

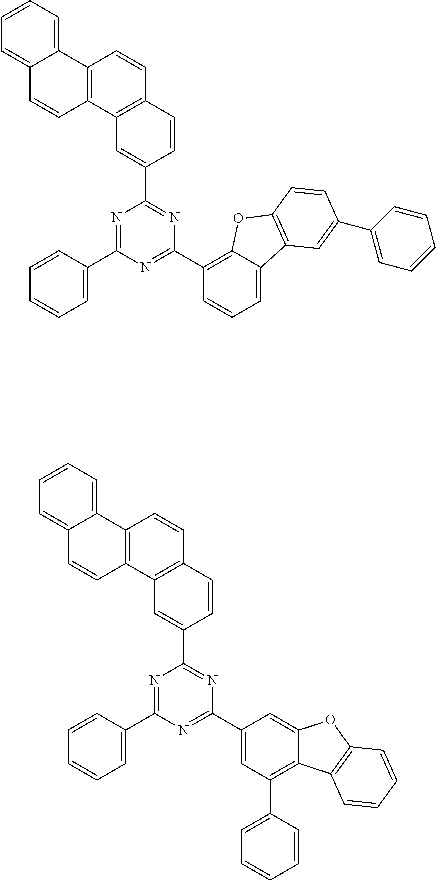 [SMILES: c1ccc(-c2ccc3oc4c(-c5nc(-c6ccccc6)nc(-c6ccc7ccc8c9ccccc9ccc8c7c6)n5)cccc4c3c2)cc1 ; c1ccc(-c2nc(-c3cc(-c4ccccc4)c4c(c3)oc3ccccc34)nc(-c3ccc4ccc5c6ccccc6ccc5c4c3)n2)cc1]

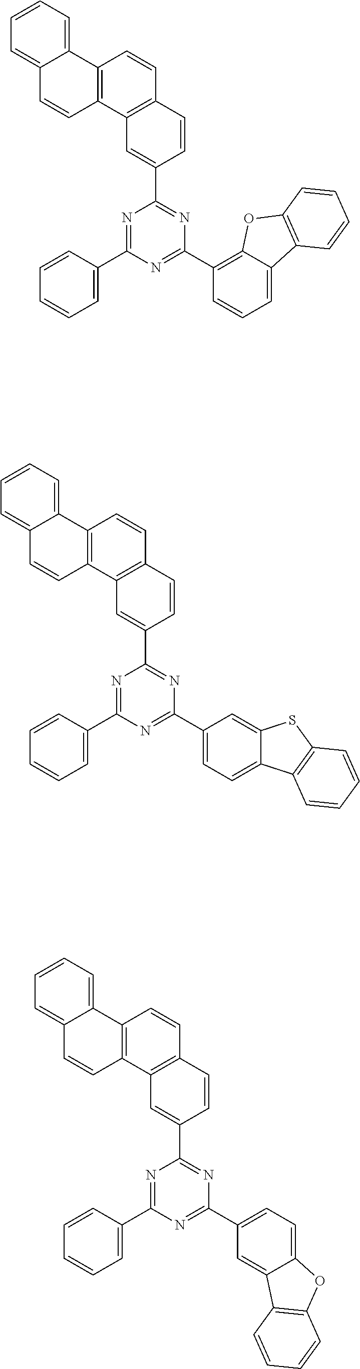 [SMILES: c1ccc(-c2nc(-c3ccc4ccc5c6ccccc6ccc5c4c3)nc(-c3cccc4c3oc3ccccc34)n2)cc1 ; c1ccc(-c2nc(-c3ccc4c(c3)sc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3ccc4oc5ccccc5c4c3)n2)cc1]

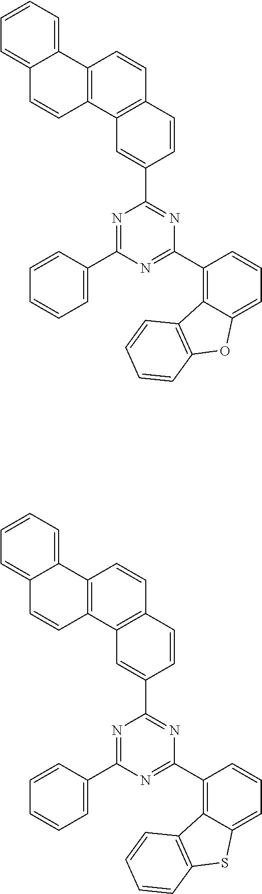 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1]

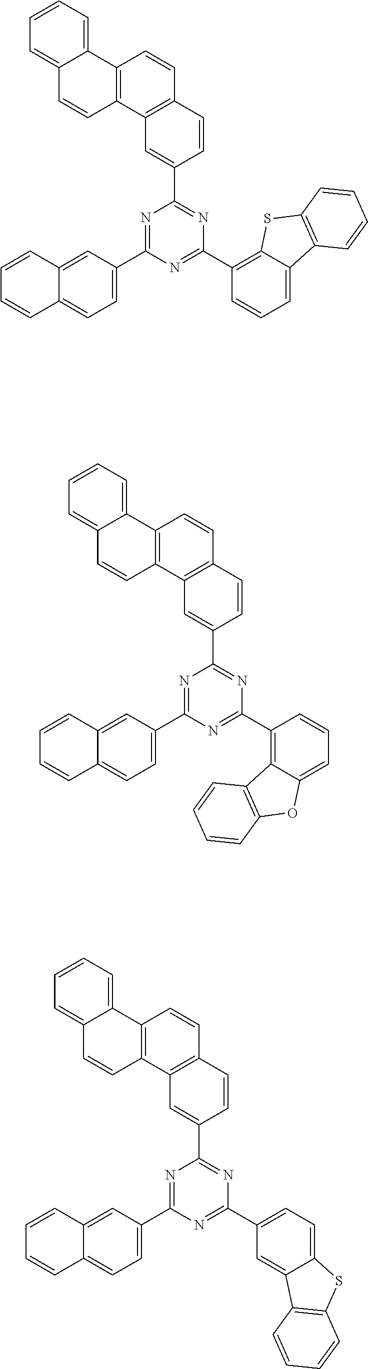 [SMILES: c1ccc2cc(-c3nc(-c4ccc5ccc6c7ccccc7ccc6c5c4)nc(-c4cccc5c4sc4ccccc45)n3)ccc2c1 ; c1ccc2cc(-c3nc(-c4ccc5ccc6c7ccccc7ccc6c5c4)nc(-c4cccc5oc6ccccc6c45)n3)ccc2c1 ; c1ccc2cc(-c3nc(-c4ccc5ccc6c7ccccc7ccc6c5c4)nc(-c4ccc5sc6ccccc6c5c4)n3)ccc2c1]

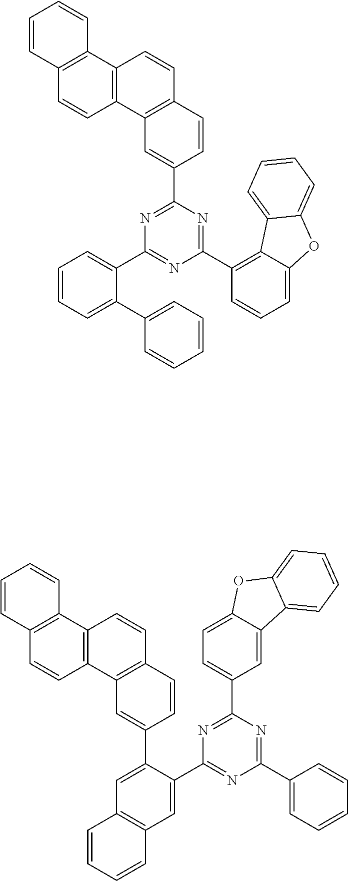 [SMILES: c1ccc(-c2ccccc2-c2nc(-c3ccc4ccc5c6ccccc6ccc5c4c3)nc(-c3cccc4oc5ccccc5c34)n2)cc1 ; c1ccc(-c2nc(-c3ccc4oc5ccccc5c4c3)nc(-c3cc4ccccc4cc3-c3ccc4ccc5c6ccccc6ccc5c4c3)n2)cc1]

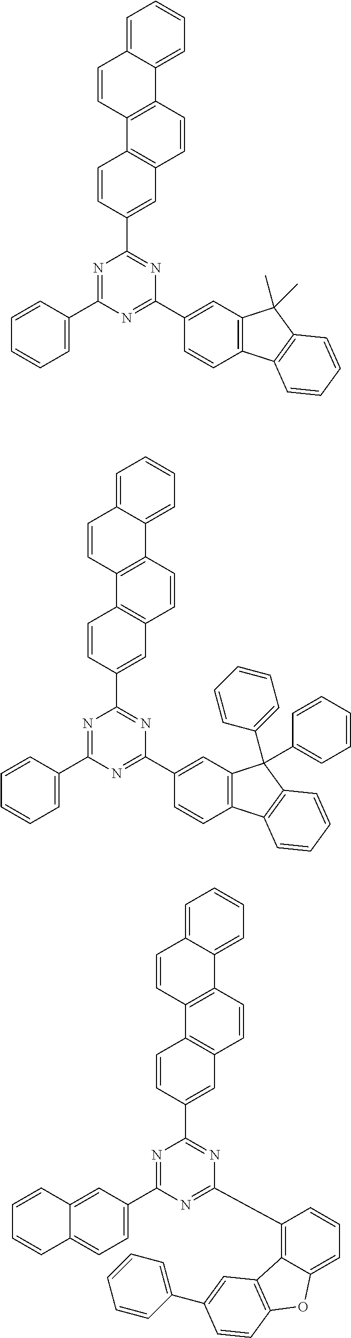 [SMILES: CC1(C)c2ccccc2-c2ccc(-c3nc(-c4ccccc4)nc(-c4ccc5c(ccc6c7ccccc7ccc56)c4)n3)cc21 ; c1ccc(-c2nc(-c3ccc4c(c3)C(c3ccccc3)(c3ccccc3)c3ccccc3-4)nc(-c3ccc4c(ccc5c6ccccc6ccc45)c3)n2)cc1 ; c1ccc(-c2ccc3oc4cccc(-c5nc(-c6ccc7ccccc7c6)nc(-c6ccc7c(ccc8c9ccccc9ccc78)c6)n5)c4c3c2)cc1]

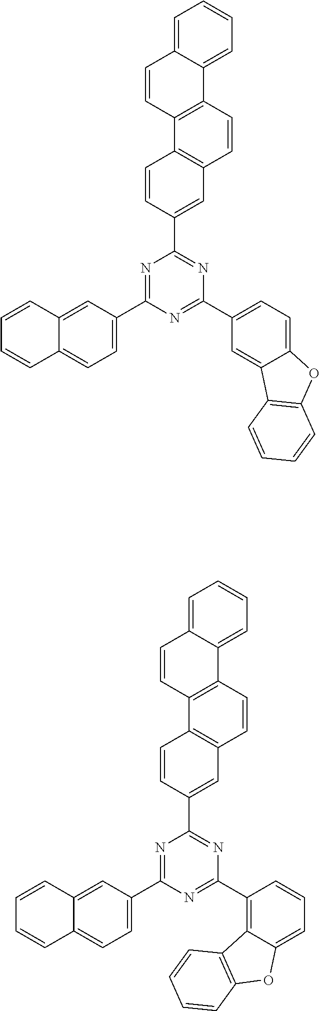 [SMILES: c1ccc2cc(-c3nc(-c4ccc5c(ccc6c7ccccc7ccc56)c4)nc(-c4ccc5oc6ccccc6c5c4)n3)ccc2c1 ; c1ccc2cc(-c3nc(-c4ccc5c(ccc6c7ccccc7ccc56)c4)nc(-c4cccc5oc6ccccc6c45)n3)ccc2c1]

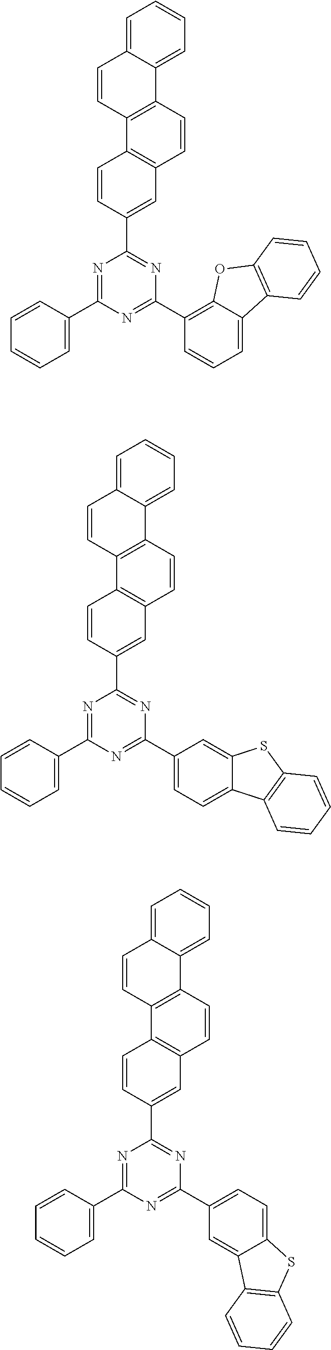 [SMILES: c1ccc(-c2nc(-c3ccc4c(ccc5c6ccccc6ccc45)c3)nc(-c3cccc4c3oc3ccccc34)n2)cc1 ; c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3ccc4sc5ccccc5c4c3)n2)cc1]

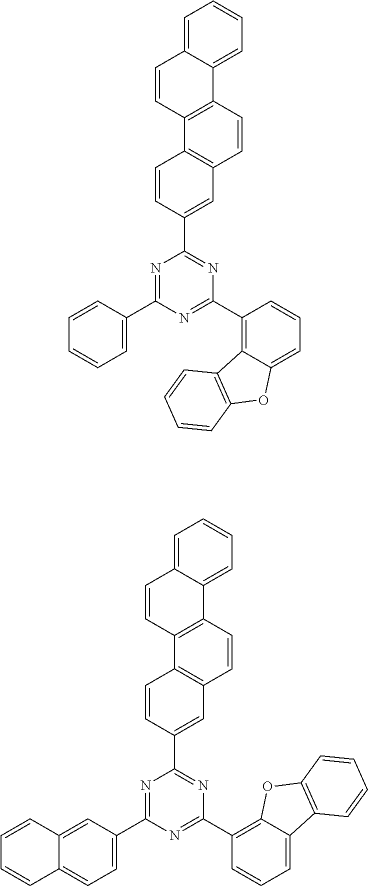 [SMILES: c1ccc(-c2nc(-c3ccc4c(ccc5c6ccccc6ccc45)c3)nc(-c3cccc4oc5ccccc5c34)n2)cc1 ; c1ccc2cc(-c3nc(-c4ccc5c(ccc6c7ccccc7ccc56)c4)nc(-c4cccc5c4oc4ccccc45)n3)ccc2c1]

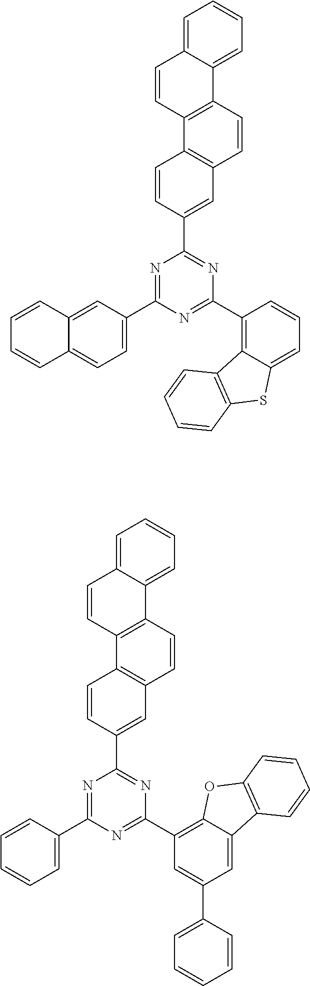 [SMILES: c1ccc2cc(-c3nc(-c4ccc5c(ccc6c7ccccc7ccc56)c4)nc(-c4cccc5sc6ccccc6c45)n3)ccc2c1 ; c1ccc(-c2cc(-c3nc(-c4ccccc4)nc(-c4ccc5c(ccc6c7ccccc7ccc56)c4)n3)c3oc4ccccc4c3c2)cc1]

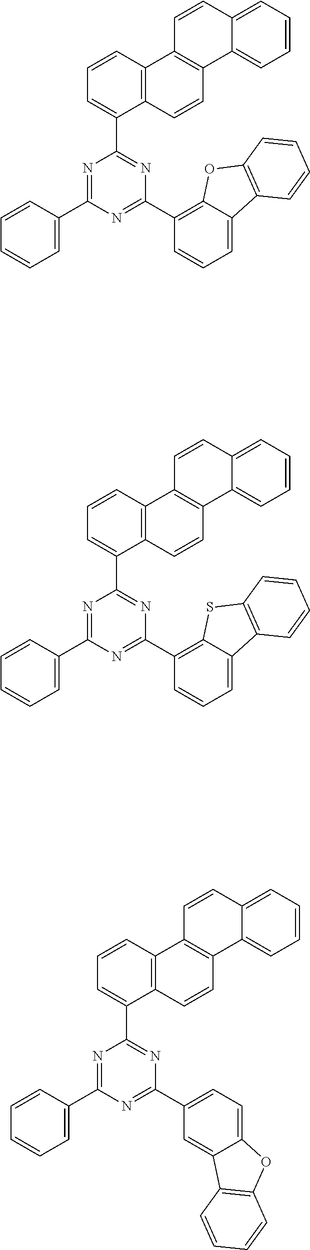 [SMILES: c1ccc(-c2nc(-c3cccc4c3ccc3c5ccccc5ccc43)nc(-c3cccc4c3oc3ccccc34)n2)cc1 ; c1ccc(-c2nc(-c3cccc4c3ccc3c5ccccc5ccc43)nc(-c3cccc4c3sc3ccccc34)n2)cc1 ; c1ccc(-c2nc(-c3ccc4oc5ccccc5c4c3)nc(-c3cccc4c3ccc3c5ccccc5ccc43)n2)cc1]

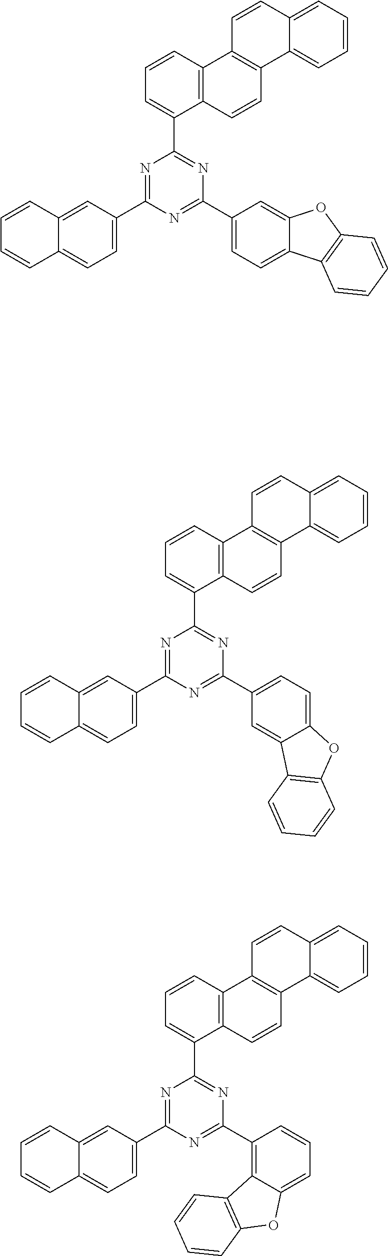 [SMILES: c1ccc2cc(-c3nc(-c4ccc5c(c4)oc4ccccc45)nc(-c4cccc5c4ccc4c6ccccc6ccc54)n3)ccc2c1 ; c1ccc2cc(-c3nc(-c4ccc5oc6ccccc6c5c4)nc(-c4cccc5c4ccc4c6ccccc6ccc54)n3)ccc2c1 ; c1ccc2cc(-c3nc(-c4cccc5c4ccc4c6ccccc6ccc54)nc(-c4cccc5oc6ccccc6c45)n3)ccc2c1]

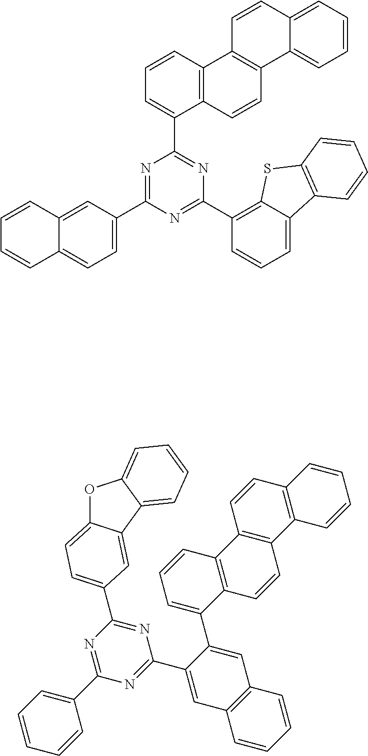 [SMILES: c1ccc2cc(-c3nc(-c4cccc5c4ccc4c6ccccc6ccc54)nc(-c4cccc5c4sc4ccccc45)n3)ccc2c1 ; c1ccc(-c2nc(-c3ccc4oc5ccccc5c4c3)nc(-c3cc4ccccc4cc3-c3cccc4c3ccc3c5ccccc5ccc43)n2)cc1]

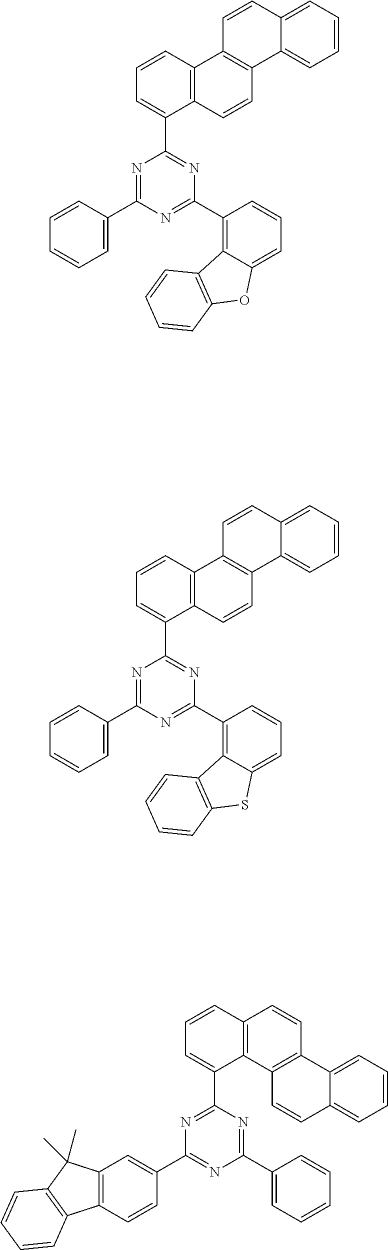 [SMILES: c1ccc(-c2nc(-c3cccc4c3ccc3c5ccccc5ccc43)nc(-c3cccc4oc5ccccc5c34)n2)cc1 ; c1ccc(-c2nc(-c3cccc4c3ccc3c5ccccc5ccc43)nc(-c3cccc4sc5ccccc5c34)n2)cc1 ; CC1(C)c2ccccc2-c2ccc(-c3nc(-c4ccccc4)nc(-c4cccc5ccc6c7ccccc7ccc6c45)n3)cc21]

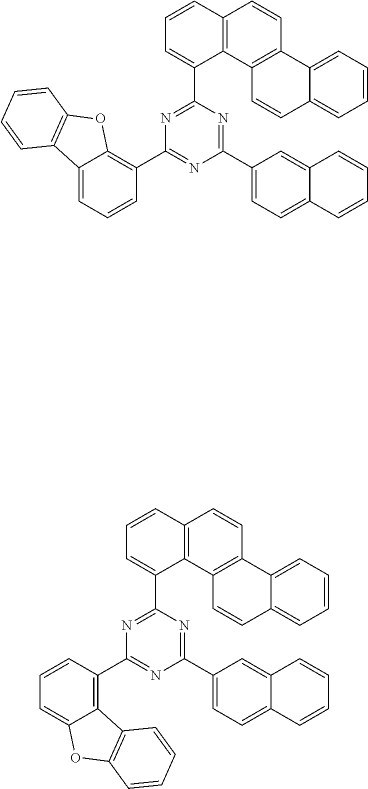 [SMILES: c1ccc2cc(-c3nc(-c4cccc5c4oc4ccccc45)nc(-c4cccc5ccc6c7ccccc7ccc6c45)n3)ccc2c1 ; c1ccc2cc(-c3nc(-c4cccc5ccc6c7ccccc7ccc6c45)nc(-c4cccc5oc6ccccc6c45)n3)ccc2c1]

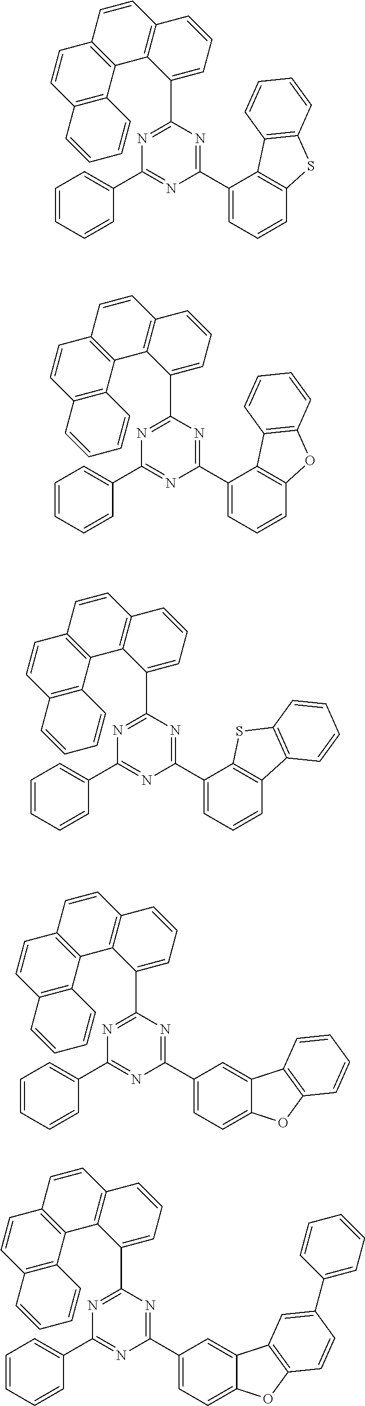 [SMILES: c1ccc(-c2nc(-c3cccc4sc5ccccc5c34)nc(-c3cccc4ccc5ccc6ccccc6c5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3cccc4c3sc3ccccc34)nc(-c3cccc4ccc5ccc6ccccc6c5c34)n2)cc1 ; c1ccc(-c2nc(-c3ccc4oc5ccccc5c4c3)nc(-c3cccc4ccc5ccc6ccccc6c5c34)n2)cc1 ; c1ccc(-c2ccc3oc4ccc(-c5nc(-c6ccccc6)nc(-c6cccc7ccc8ccc9ccccc9c8c67)n5)cc4c3c2)cc1]

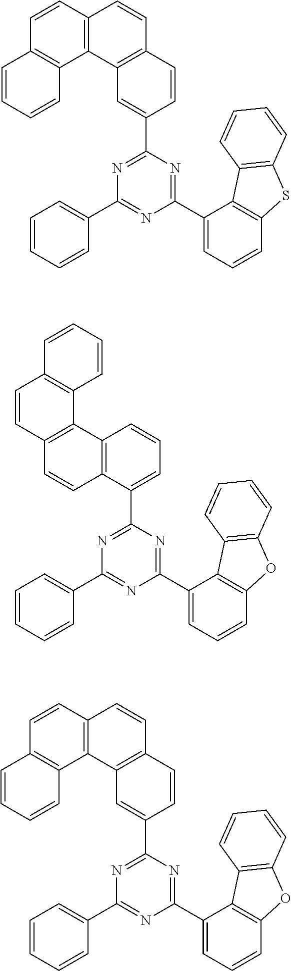 [SMILES: c1ccc(-c2nc(-c3ccc4ccc5ccc6ccccc6c5c4c3)nc(-c3cccc4sc5ccccc5c34)n2)cc1 ; c1ccc(-c2nc(-c3cccc4c3ccc3ccc5ccccc5c34)nc(-c3cccc4oc5ccccc5c34)n2)cc1 ; c1ccc(-c2nc(-c3ccc4ccc5ccc6ccccc6c5c4c3)nc(-c3cccc4oc5ccccc5c34)n2)cc1]

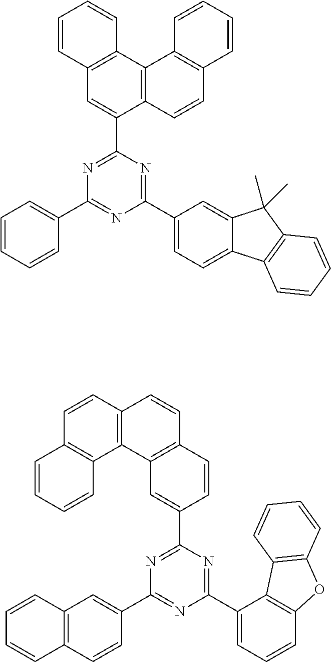 [SMILES: CC1(C)c2ccccc2-c2ccc(-c3nc(-c4ccccc4)nc(-c4cc5ccccc5c5c4ccc4ccccc45)n3)cc21 ; c1ccc2cc(-c3nc(-c4ccc5ccc6ccc7ccccc7c6c5c4)nc(-c4cccc5oc6ccccc6c45)n3)ccc2c1]

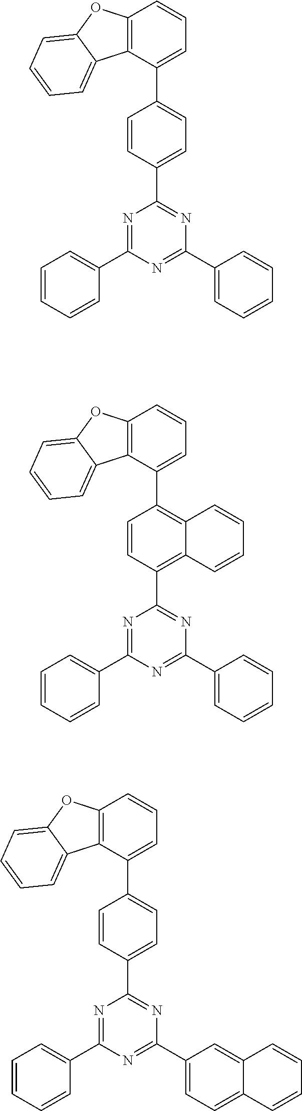 [SMILES: c1ccc(-c2nc(-c3ccccc3)nc(-c3ccc(-c4cccc5oc6ccccc6c45)cc3)n2)cc1 ; c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1]

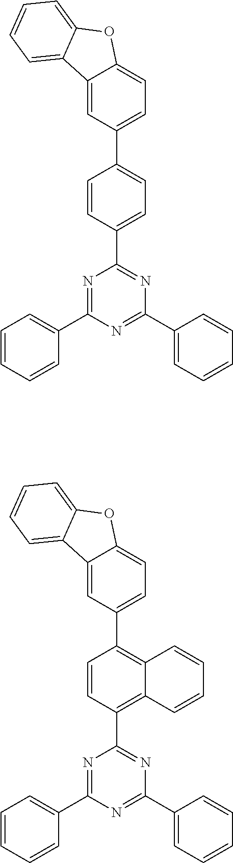 [SMILES: c1ccc(-c2nc(-c3ccccc3)nc(-c3ccc(-c4ccc5oc6ccccc6c5c4)cc3)n2)cc1 ; c1ccc(-c2nc(-c3ccccc3)nc(-c3ccc(-c4ccc5oc6ccccc6c5c4)c4ccccc34)n2)cc1]

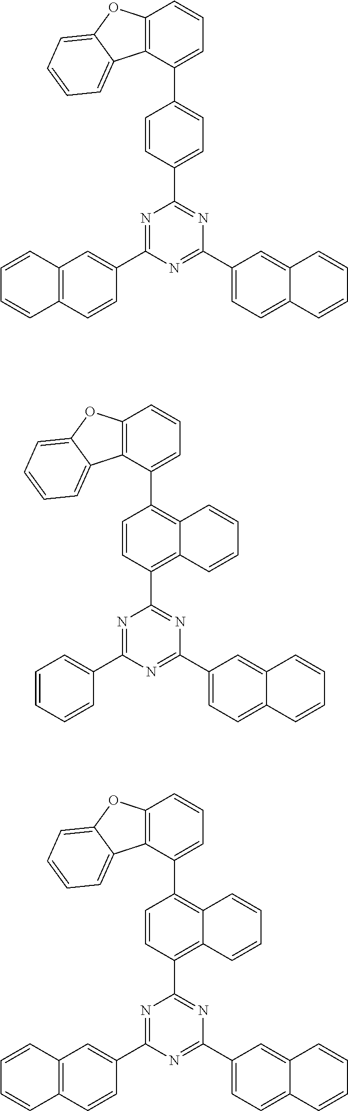 [SMILES: c1ccc2cc(-c3nc(-c4ccc(-c5cccc6oc7ccccc7c56)cc4)nc(-c4ccc5ccccc5c4)n3)ccc2c1 ; c1ccc(-c2nc(-c3ccc4ccccc4c3)nc(-c3ccc(-c4cccc5oc6ccccc6c45)c4ccccc34)n2)cc1 ; c1ccc2cc(-c3nc(-c4ccc5ccccc5c4)nc(-c4ccc(-c5cccc6oc7ccccc7c56)c5ccccc45)n3)ccc2c1]

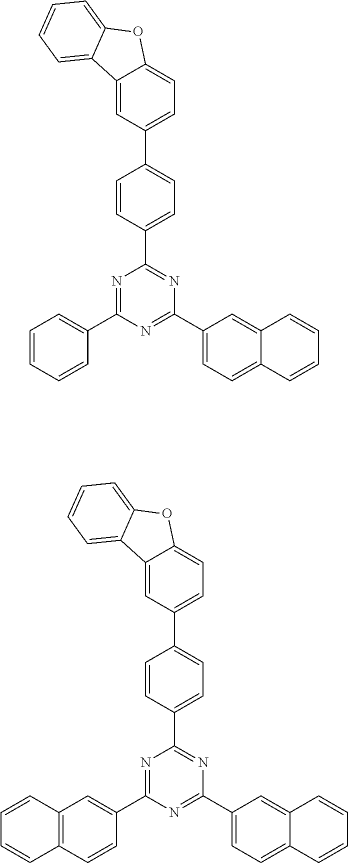 [SMILES: c1ccc(-c2nc(-c3ccc(-c4ccc5oc6ccccc6c5c4)cc3)nc(-c3ccc4ccccc4c3)n2)cc1 ; c1ccc2cc(-c3nc(-c4ccc(-c5ccc6oc7ccccc7c6c5)cc4)nc(-c4ccc5ccccc5c4)n3)ccc2c1]

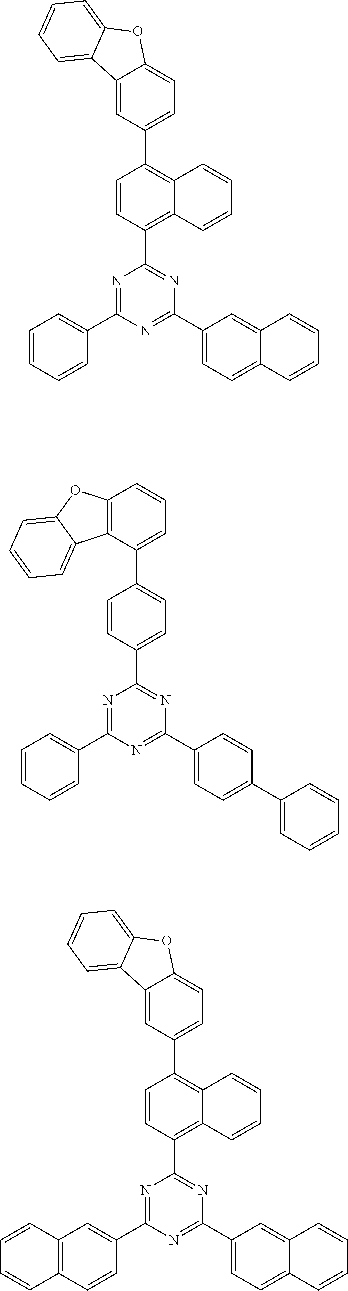 [SMILES: c1ccc(-c2nc(-c3ccc4ccccc4c3)nc(-c3ccc(-c4ccc5oc6ccccc6c5c4)c4ccccc34)n2)cc1 ; c1ccc(-c2ccc(-c3nc(-c4ccccc4)nc(-c4ccc(-c5cccc6oc7ccccc7c56)cc4)n3)cc2)cc1 ; c1ccc2cc(-c3nc(-c4ccc5ccccc5c4)nc(-c4ccc(-c5ccc6oc7ccccc7c6c5)c5ccccc45)n3)ccc2c1]

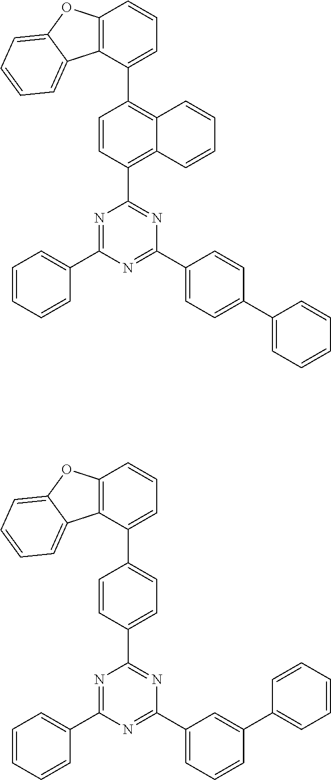 [SMILES: c1ccc(-c2ccc(-c3nc(-c4ccccc4)nc(-c4ccc(-c5cccc6oc7ccccc7c56)c5ccccc45)n3)cc2)cc1 ; c1ccc(-c2cccc(-c3nc(-c4ccccc4)nc(-c4ccc(-c5cccc6oc7ccccc7c56)cc4)n3)c2)cc1]

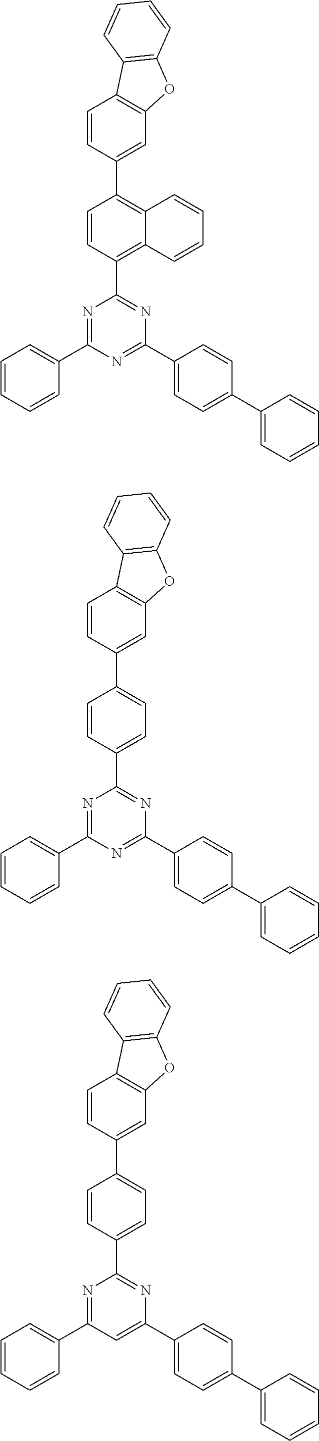 [SMILES: c1ccc(-c2ccc(-c3nc(-c4ccccc4)nc(-c4ccc(-c5ccc6c(c5)oc5ccccc56)c5ccccc45)n3)cc2)cc1 ; c1ccc(-c2ccc(-c3nc(-c4ccccc4)nc(-c4ccc(-c5ccc6c(c5)oc5ccccc56)cc4)n3)cc2)cc1 ; c1ccc(-c2ccc(-c3cc(-c4ccccc4)nc(-c4ccc(-c5ccc6c(c5)oc5ccccc56)cc4)n3)cc2)cc1]

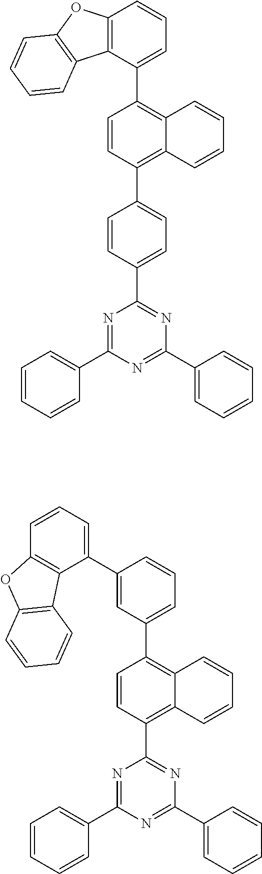 [SMILES: c1ccc(-c2nc(-c3ccccc3)nc(-c3ccc(-c4ccc(-c5cccc6oc7ccccc7c56)c5ccccc45)cc3)n2)cc1 ; c1ccc(-c2nc(-c3ccccc3)nc(-c3ccc(-c4cccc(-c5cccc6oc7ccccc7c56)c4)c4ccccc34)n2)cc1]

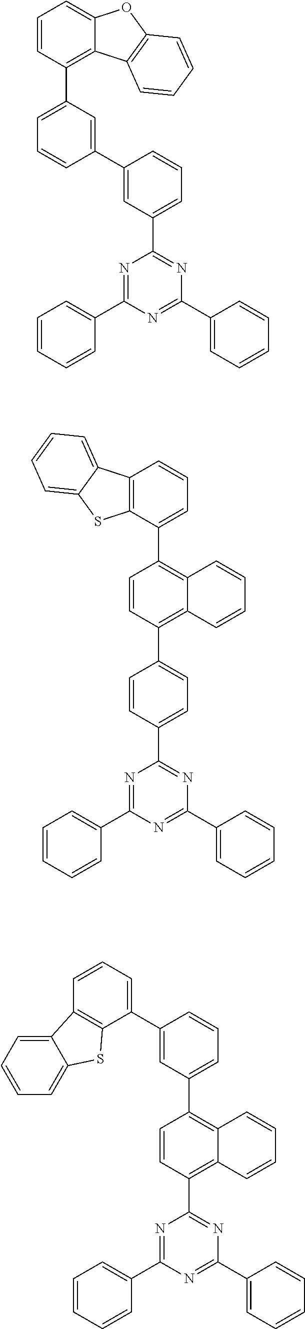 [SMILES: c1ccc(-c2nc(-c3ccccc3)nc(-c3cccc(-c4cccc(-c5cccc6oc7ccccc7c56)c4)c3)n2)cc1 ; c1ccc(-c2nc(-c3ccccc3)nc(-c3ccc(-c4ccc(-c5cccc6c5sc5ccccc56)c5ccccc45)cc3)n2)cc1 ; c1ccc(-c2nc(-c3ccccc3)nc(-c3ccc(-c4cccc(-c5cccc6c5sc5ccccc56)c4)c4ccccc34)n2)cc1]

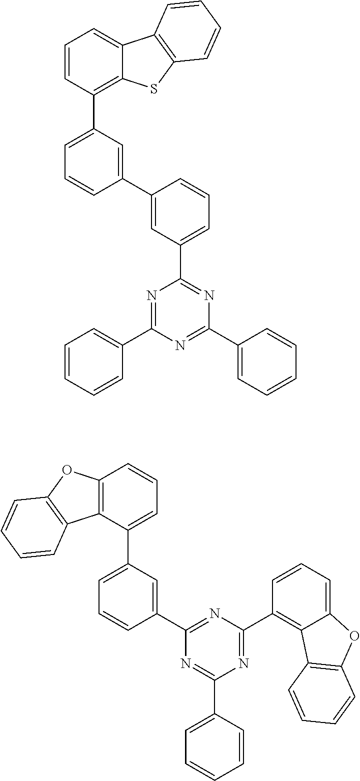 [SMILES: c1ccc(-c2nc(-c3ccccc3)nc(-c3cccc(-c4cccc(-c5cccc6c5sc5ccccc56)c4)c3)n2)cc1 ; c1ccc(-c2nc(-c3cccc(-c4cccc5oc6ccccc6c45)c3)nc(-c3cccc4oc5ccccc5c34)n2)cc1]

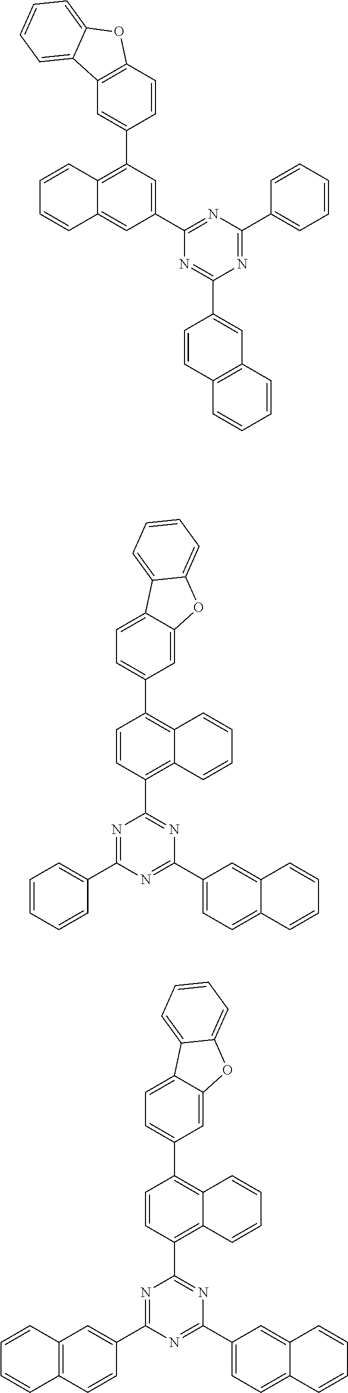 [SMILES: c1ccc(-c2nc(-c3ccc4ccccc4c3)nc(-c3cc(-c4ccc5oc6ccccc6c5c4)c4ccccc4c3)n2)cc1 ; c1ccc(-c2nc(-c3ccc4ccccc4c3)nc(-c3ccc(-c4ccc5c(c4)oc4ccccc45)c4ccccc34)n2)cc1 ; c1ccc2cc(-c3nc(-c4ccc5ccccc5c4)nc(-c4ccc(-c5ccc6c(c5)oc5ccccc56)c5ccccc45)n3)ccc2c1]

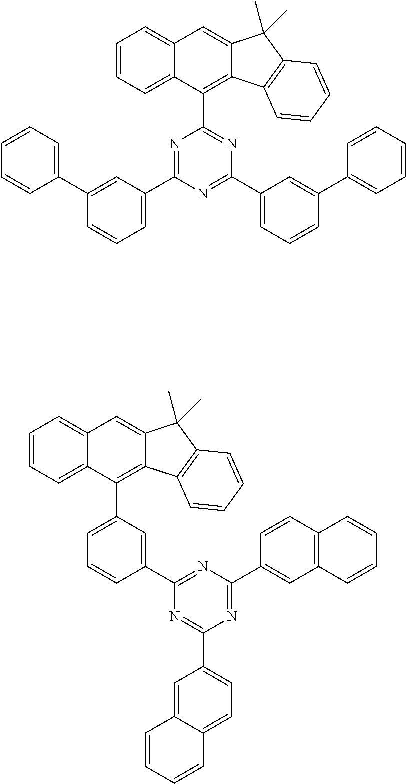 [SMILES: CC1(C)c2ccccc2-c2c1cc1ccccc1c2-c1nc(-c2cccc(-c3ccccc3)c2)nc(-c2cccc(-c3ccccc3)c2)n1 ; CC1(C)c2ccccc2-c2c1cc1ccccc1c2-c1cccc(-c2nc(-c3ccc4ccccc4c3)nc(-c3ccc4ccccc4c3)n2)c1]

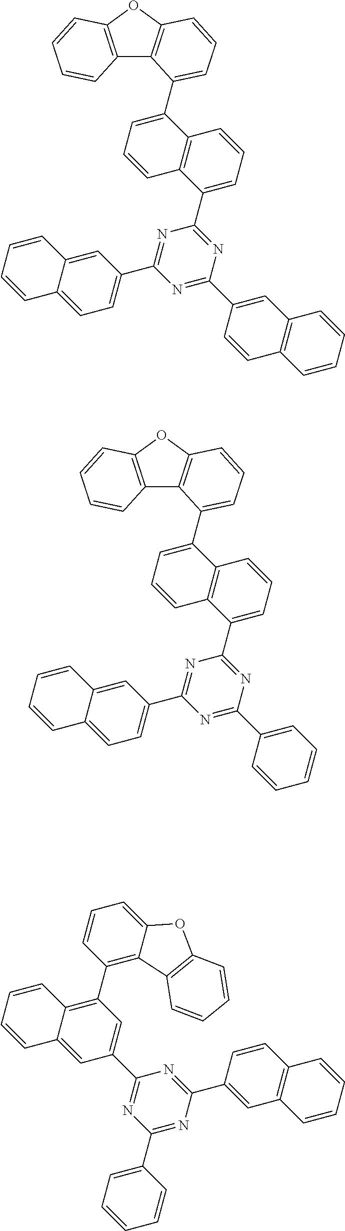 [SMILES: c1ccc2cc(-c3nc(-c4ccc5ccccc5c4)nc(-c4cccc5c(-c6cccc7oc8ccccc8c67)cccc45)n3)ccc2c1 ; c1ccc(-c2nc(-c3ccc4ccccc4c3)nc(-c3cccc4c(-c5cccc6oc7ccccc7c56)cccc34)n2)cc1 ; c1ccc(-c2nc(-c3ccc4ccccc4c3)nc(-c3cc(-c4cccc5oc6ccccc6c45)c4ccccc4c3)n2)cc1]

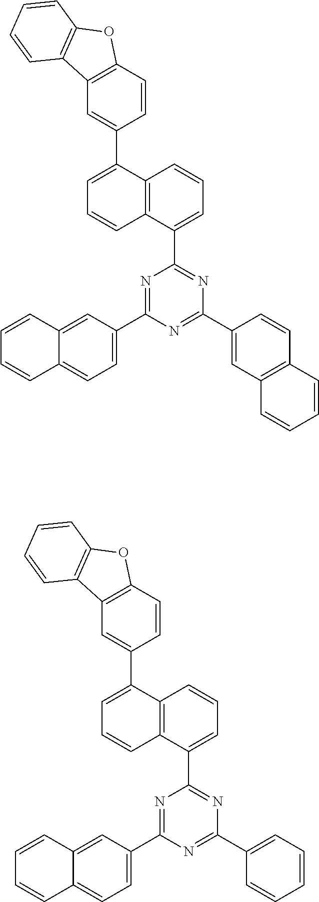 [SMILES: c1ccc2cc(-c3nc(-c4ccc5ccccc5c4)nc(-c4cccc5c(-c6ccc7oc8ccccc8c7c6)cccc45)n3)ccc2c1 ; c1ccc(-c2nc(-c3ccc4ccccc4c3)nc(-c3cccc4c(-c5ccc6oc7ccccc7c6c5)cccc34)n2)cc1]

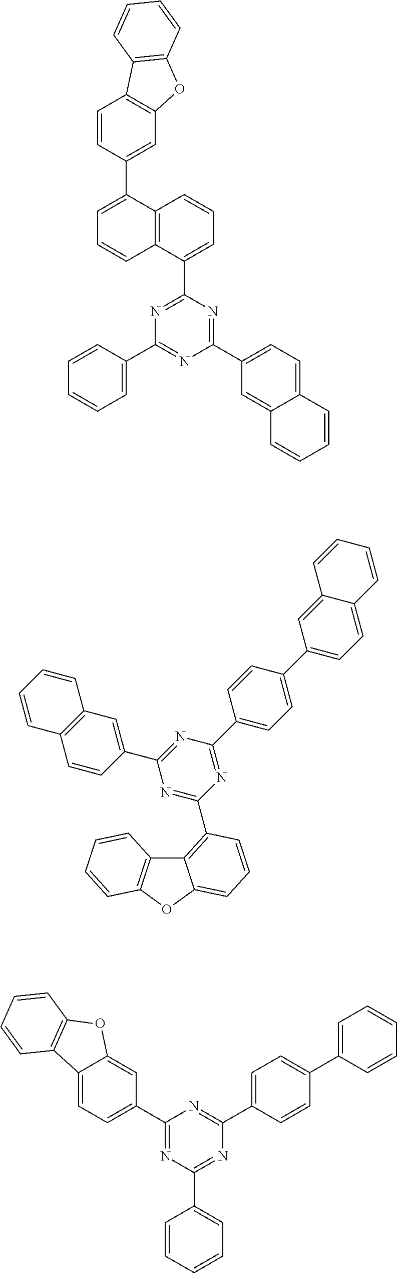 [SMILES: c1ccc(-c2nc(-c3ccc4ccccc4c3)nc(-c3cccc4c(-c5ccc6c(c5)oc5ccccc56)cccc34)n2)cc1 ; c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3nc(-c4ccccc4)nc(-c4ccc5c(c4)oc4ccccc45)n3)cc2)cc1]

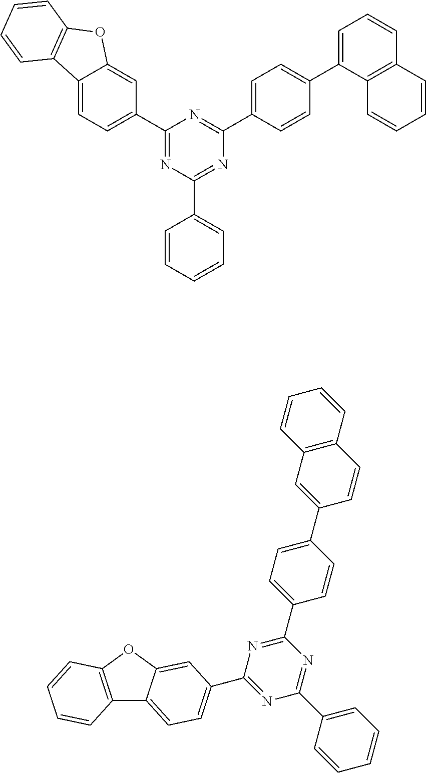 [SMILES: c1ccc(-c2nc(-c3ccc(-c4cccc5ccccc45)cc3)nc(-c3ccc4c(c3)oc3ccccc34)n2)cc1 ; c1ccc(-c2nc(-c3ccc(-c4ccc5ccccc5c4)cc3)nc(-c3ccc4c(c3)oc3ccccc34)n2)cc1]

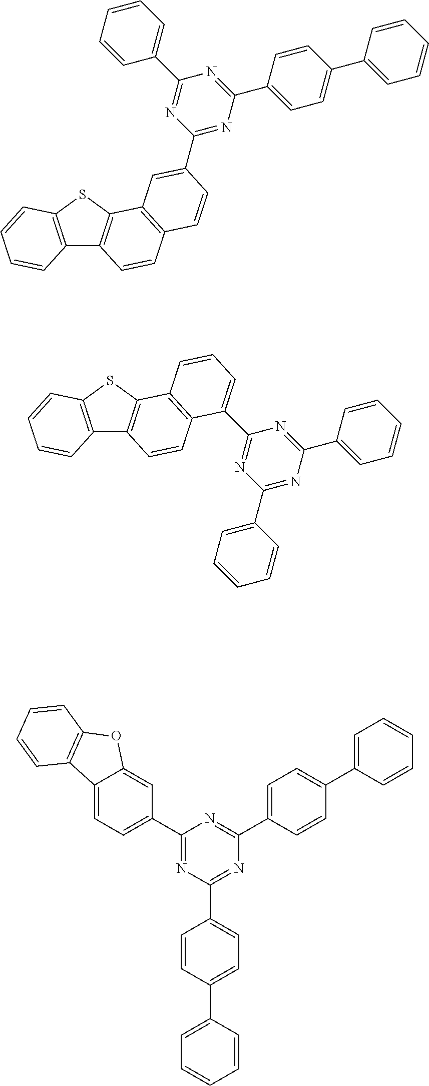 [SMILES: c1ccc(-c2ccc(-c3nc(-c4ccccc4)nc(-c4ccc5ccc6c7ccccc7sc6c5c4)n3)cc2)cc1 ; c1ccc(-c2nc(-c3ccccc3)nc(-c3cccc4c3ccc3c5ccccc5sc43)n2)cc1 ; c1ccc(-c2ccc(-c3nc(-c4ccc(-c5ccccc5)cc4)nc(-c4ccc5c(c4)oc4ccccc45)n3)cc2)cc1]

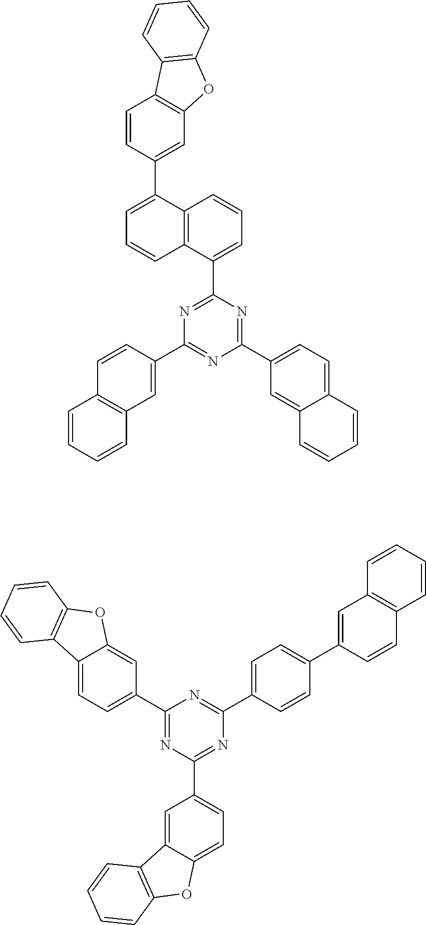 [SMILES: c1ccc2cc(-c3nc(-c4ccc5ccccc5c4)nc(-c4cccc5c(-c6ccc7c(c6)oc6ccccc67)cccc45)n3)ccc2c1 ; c1ccc2cc(-c3ccc(-c4nc(-c5ccc6c(c5)oc5ccccc56)nc(-c5ccc6oc7ccccc7c6c5)n4)cc3)ccc2c1]

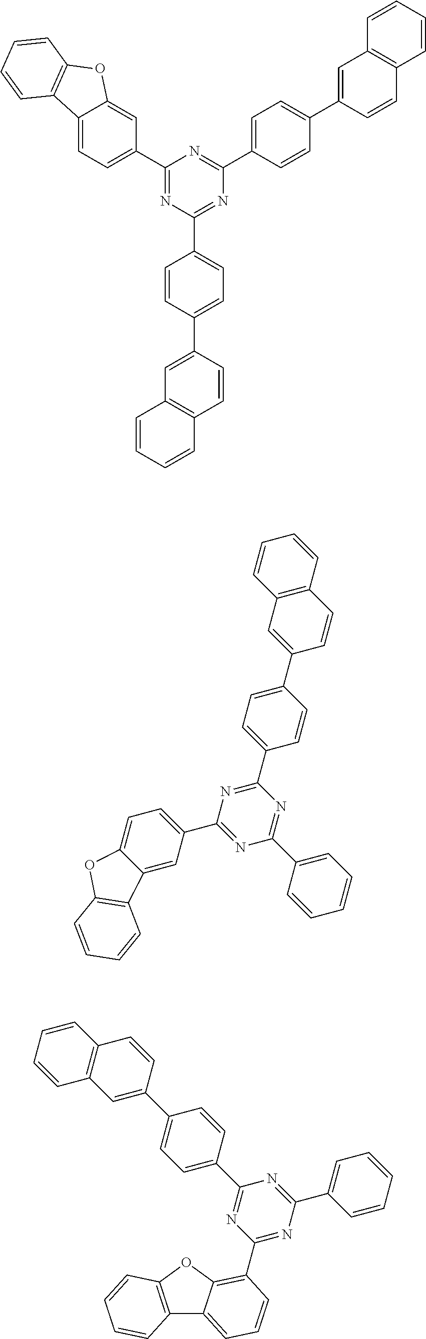 [SMILES: c1ccc2cc(-c3ccc(-c4nc(-c5ccc(-c6ccc7ccccc7c6)cc5)nc(-c5ccc6c(c5)oc5ccccc56)n4)cc3)ccc2c1 ; c1ccc(-c2nc(-c3ccc(-c4ccc5ccccc5c4)cc3)nc(-c3ccc4oc5ccccc5c4c3)n2)cc1 ; c1ccc(-c2nc(-c3ccc(-c4ccc5ccccc5c4)cc3)nc(-c3cccc4c3oc3ccccc34)n2)cc1]

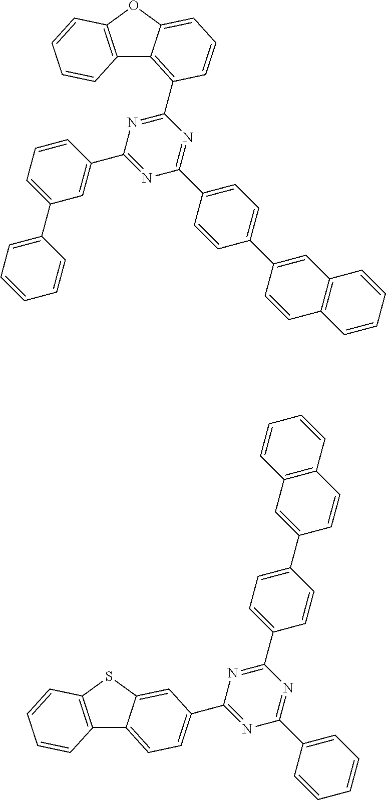 [SMILES: c1ccc(-c2cccc(-c3nc(-c4ccc(-c5ccc6ccccc6c5)cc4)nc(-c4cccc5oc6ccccc6c45)n3)c2)cc1 ; c1ccc(-c2nc(-c3ccc(-c4ccc5ccccc5c4)cc3)nc(-c3ccc4c(c3)sc3ccccc34)n2)cc1]

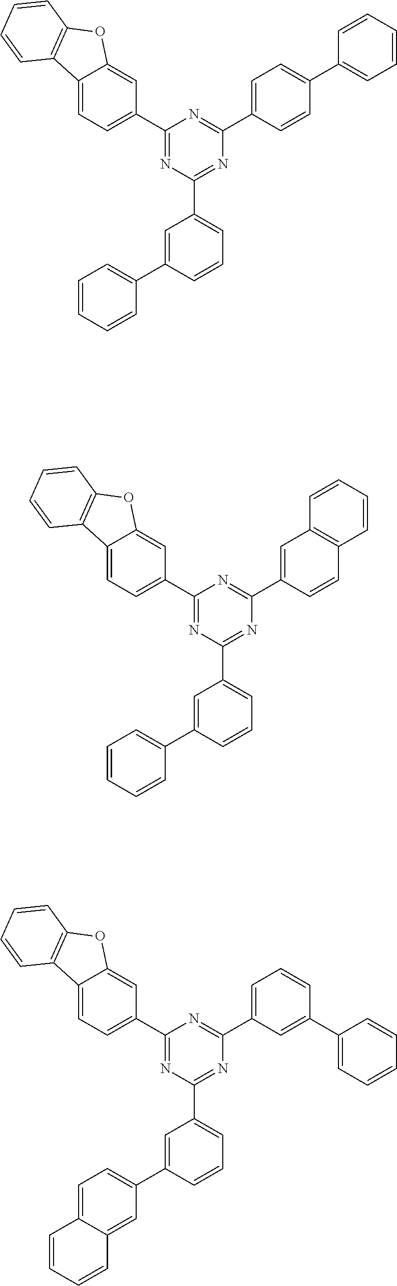 [SMILES: c1ccc(-c2ccc(-c3nc(-c4cccc(-c5ccccc5)c4)nc(-c4ccc5c(c4)oc4ccccc45)n3)cc2)cc1 ; c1ccc(-c2cccc(-c3nc(-c4ccc5ccccc5c4)nc(-c4ccc5c(c4)oc4ccccc45)n3)c2)cc1 ; c1ccc(-c2cccc(-c3nc(-c4cccc(-c5ccc6ccccc6c5)c4)nc(-c4ccc5c(c4)oc4ccccc45)n3)c2)cc1]

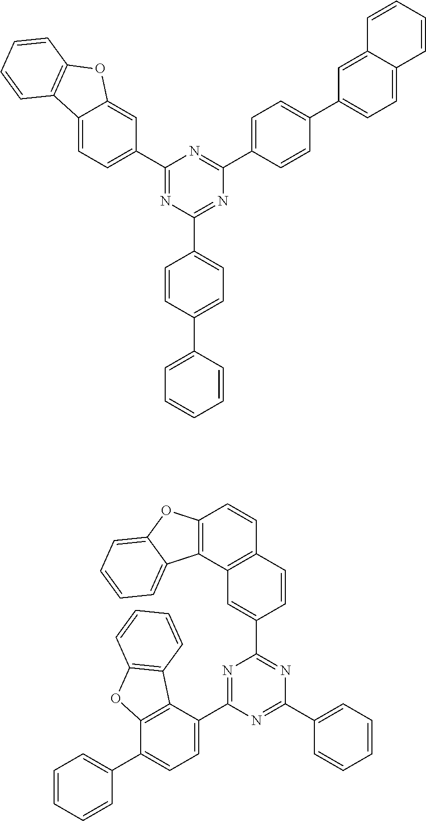 [SMILES: c1ccc(-c2ccc(-c3nc(-c4ccc(-c5ccc6ccccc6c5)cc4)nc(-c4ccc5c(c4)oc4ccccc45)n3)cc2)cc1 ; c1ccc(-c2nc(-c3ccc4ccc5oc6ccccc6c5c4c3)nc(-c3ccc(-c4ccccc4)c4oc5ccccc5c34)n2)cc1]

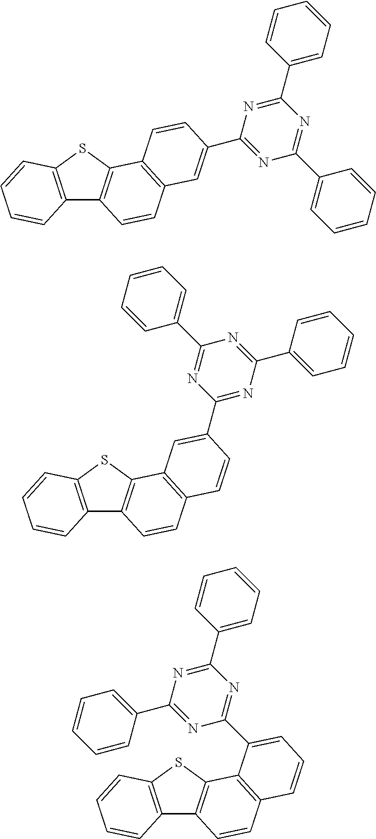 [SMILES: c1ccc(-c2nc(-c3ccccc3)nc(-c3ccc4c(ccc5c6ccccc6sc45)c3)n2)cc1 ; c1ccc(-c2nc(-c3ccccc3)nc(-c3ccc4ccc5c6ccccc6sc5c4c3)n2)cc1 ; c1ccc(-c2nc(-c3ccccc3)nc(-c3cccc4ccc5c6ccccc6sc5c34)n2)cc1]

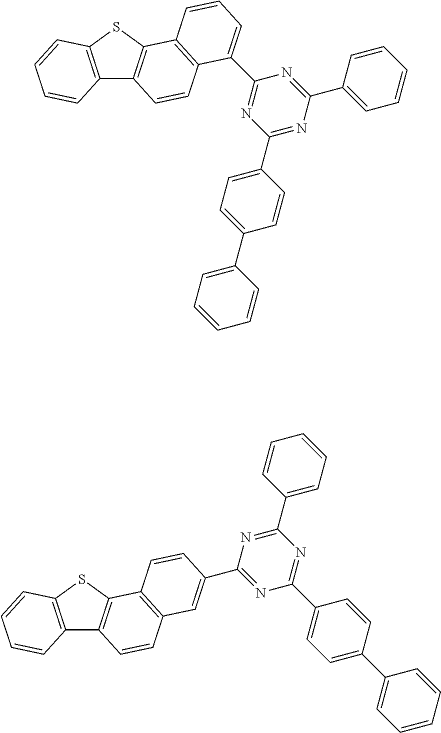 [SMILES: c1ccc(-c2ccc(-c3nc(-c4ccccc4)nc(-c4cccc5c4ccc4c6ccccc6sc54)n3)cc2)cc1 ; c1ccc(-c2ccc(-c3nc(-c4ccccc4)nc(-c4ccc5c(ccc6c7ccccc7sc56)c4)n3)cc2)cc1]

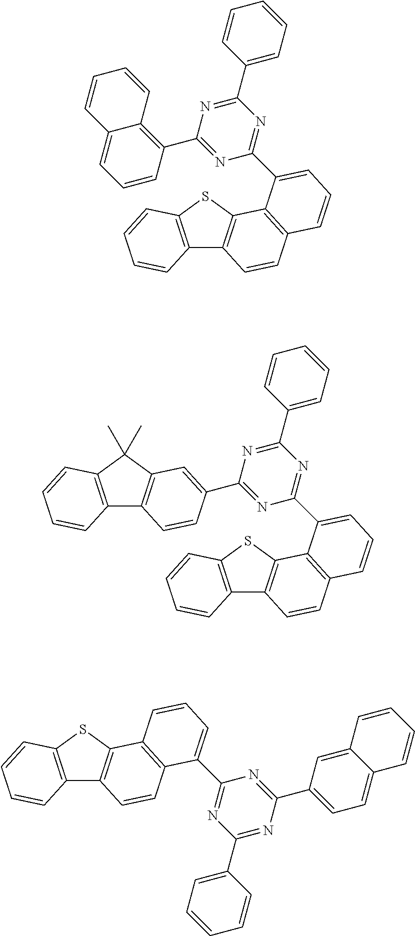 [SMILES: c1ccc(-c2nc(-c3cccc4ccccc34)nc(-c3cccc4ccc5c6ccccc6sc5c34)n2)cc1 ; CC1(C)c2ccccc2-c2ccc(-c3nc(-c4ccccc4)nc(-c4cccc5ccc6c7ccccc7sc6c45)n3)cc21 ; c1ccc(-c2nc(-c3ccc4ccccc4c3)nc(-c3cccc4c3ccc3c5ccccc5sc43)n2)cc1]

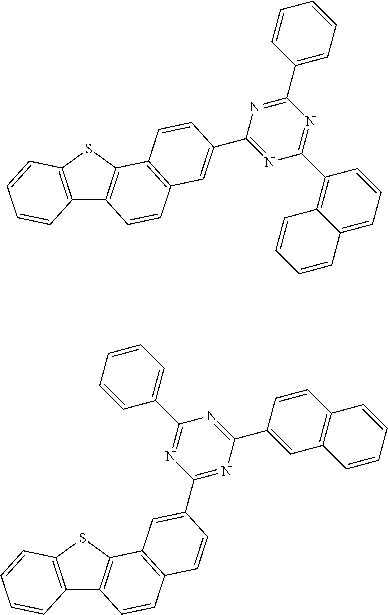 [SMILES: c1ccc(-c2nc(-c3ccc4c(ccc5c6ccccc6sc45)c3)nc(-c3cccc4ccccc34)n2)cc1 ; c1ccc(-c2nc(-c3ccc4ccccc4c3)nc(-c3ccc4ccc5c6ccccc6sc5c4c3)n2)cc1]

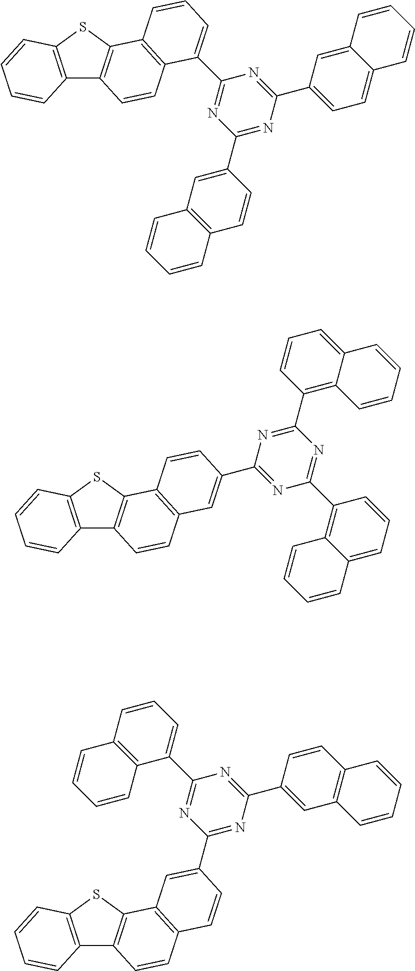 [SMILES: c1ccc2cc(-c3nc(-c4ccc5ccccc5c4)nc(-c4cccc5c4ccc4c6ccccc6sc54)n3)ccc2c1 ; c1ccc2c(-c3nc(-c4ccc5c(ccc6c7ccccc7sc56)c4)nc(-c4cccc5ccccc45)n3)cccc2c1 ; c1ccc2cc(-c3nc(-c4ccc5ccc6c7ccccc7sc6c5c4)nc(-c4cccc5ccccc45)n3)ccc2c1]

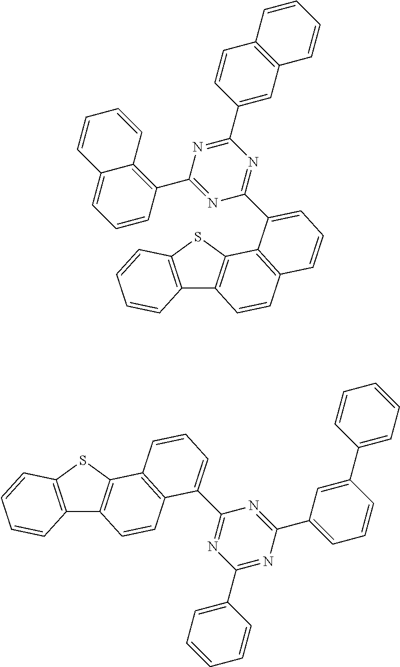 [SMILES: c1ccc2cc(-c3nc(-c4cccc5ccccc45)nc(-c4cccc5ccc6c7ccccc7sc6c45)n3)ccc2c1 ; c1ccc(-c2cccc(-c3nc(-c4ccccc4)nc(-c4cccc5c4ccc4c6ccccc6sc54)n3)c2)cc1]

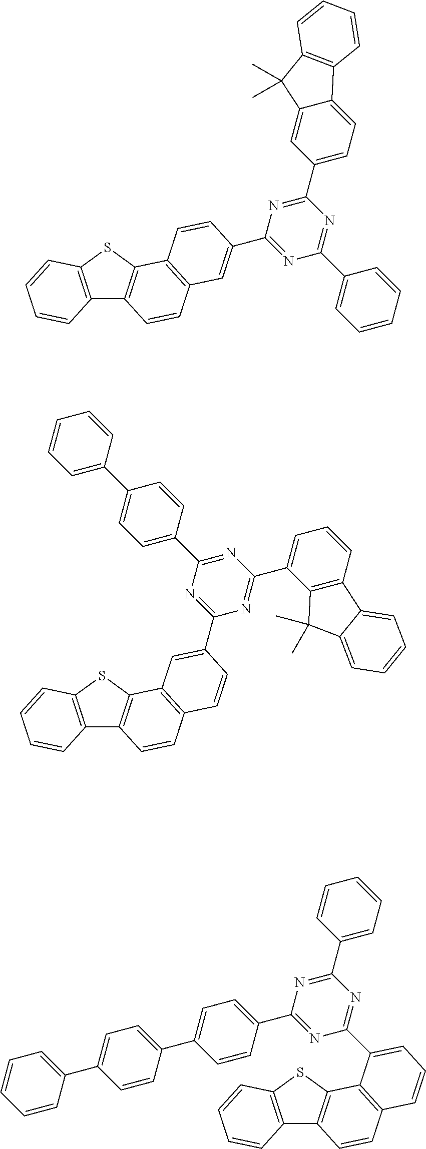 [SMILES: CC1(C)c2ccccc2-c2ccc(-c3nc(-c4ccccc4)nc(-c4ccc5c(ccc6c7ccccc7sc56)c4)n3)cc21 ; CC1(C)c2ccccc2-c2cccc(-c3nc(-c4ccc(-c5ccccc5)cc4)nc(-c4ccc5ccc6c7ccccc7sc6c5c4)n3)c21 ; c1ccc(-c2ccc(-c3ccc(-c4nc(-c5ccccc5)nc(-c5cccc6ccc7c8ccccc8sc7c56)n4)cc3)cc2)cc1]

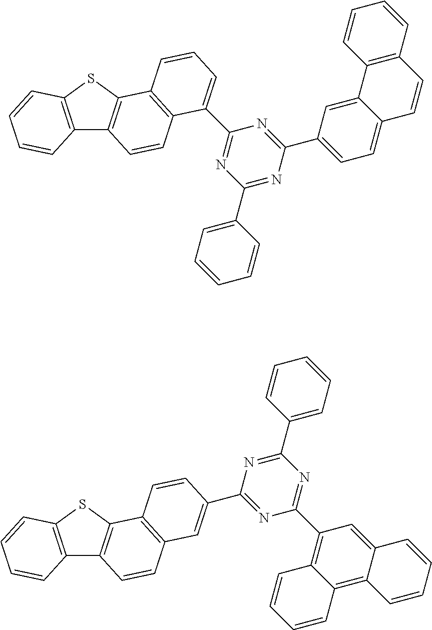 [SMILES: c1ccc(-c2nc(-c3ccc4ccc5ccccc5c4c3)nc(-c3cccc4c3ccc3c5ccccc5sc43)n2)cc1 ; c1ccc(-c2nc(-c3ccc4c(ccc5c6ccccc6sc45)c3)nc(-c3cc4ccccc4c4ccccc34)n2)cc1]

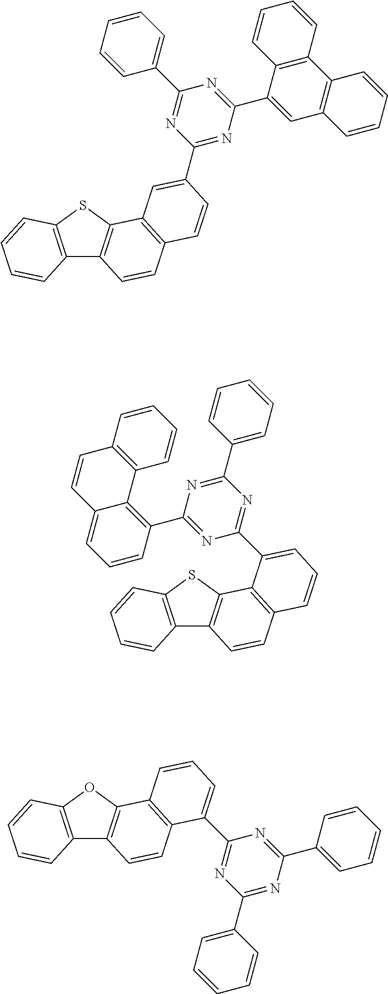 [SMILES: c1ccc(-c2nc(-c3ccc4ccc5c6ccccc6sc5c4c3)nc(-c3cc4ccccc4c4ccccc34)n2)cc1 ; c1ccc(-c2nc(-c3cccc4ccc5ccccc5c34)nc(-c3cccc4ccc5c6ccccc6sc5c34)n2)cc1 ; c1ccc(-c2nc(-c3ccccc3)nc(-c3cccc4c3ccc3c5ccccc5oc43)n2)cc1]

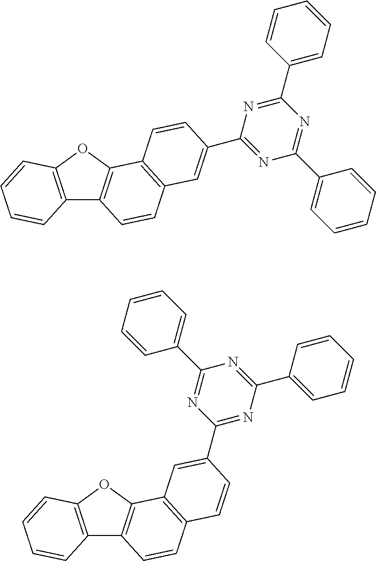 [SMILES: c1ccc(-c2nc(-c3ccccc3)nc(-c3ccc4c(ccc5c6ccccc6oc45)c3)n2)cc1 ; c1ccc(-c2nc(-c3ccccc3)nc(-c3ccc4ccc5c6ccccc6oc5c4c3)n2)cc1]

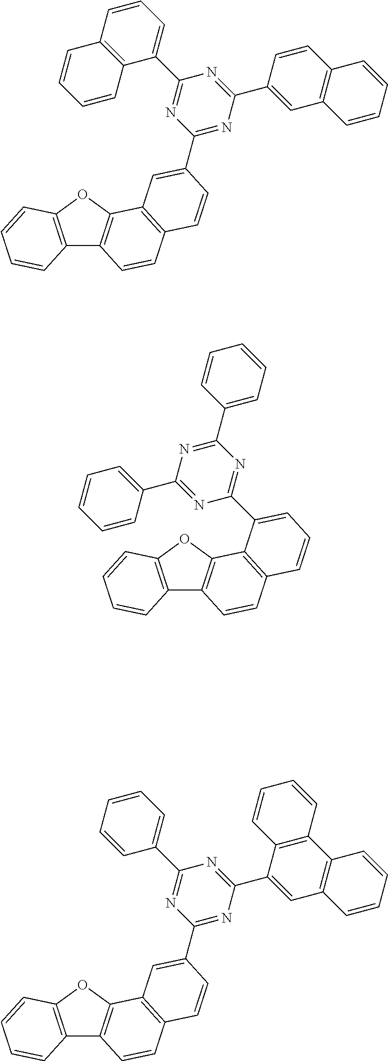 [SMILES: c1ccc2cc(-c3nc(-c4ccc5ccc6c7ccccc7oc6c5c4)nc(-c4cccc5ccccc45)n3)ccc2c1 ; c1ccc(-c2nc(-c3ccccc3)nc(-c3cccc4ccc5c6ccccc6oc5c34)n2)cc1 ; c1ccc(-c2nc(-c3ccc4ccc5c6ccccc6oc5c4c3)nc(-c3cc4ccccc4c4ccccc34)n2)cc1]

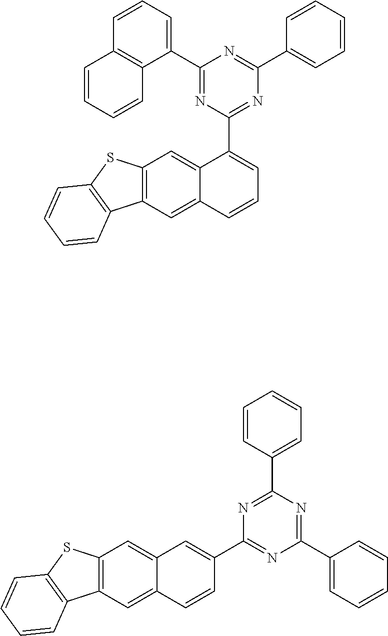 [SMILES: c1ccc(-c2nc(-c3cccc4ccccc34)nc(-c3cccc4cc5c(cc34)sc3ccccc35)n2)cc1 ; c1ccc(-c2nc(-c3ccccc3)nc(-c3ccc4cc5c(cc4c3)sc3ccccc35)n2)cc1]

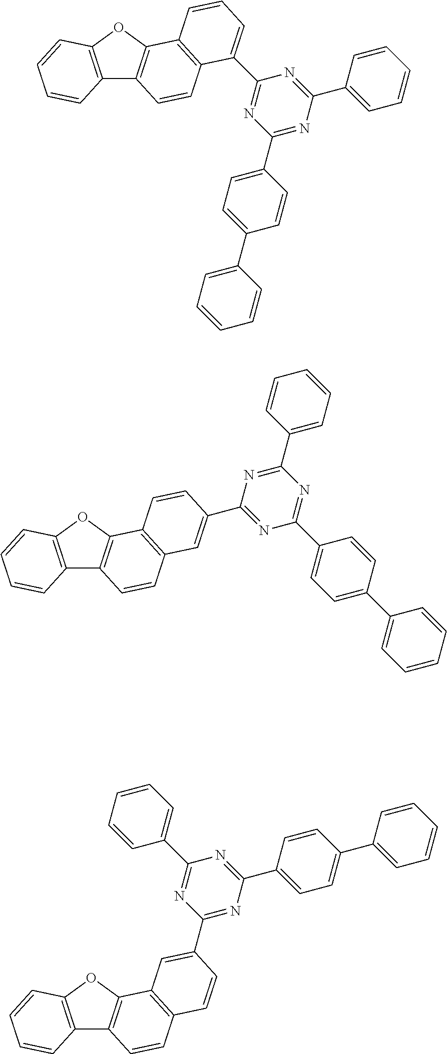 [SMILES: c1ccc(-c2ccc(-c3nc(-c4ccccc4)nc(-c4cccc5c4ccc4c6ccccc6oc54)n3)cc2)cc1 ; c1ccc(-c2ccc(-c3nc(-c4ccccc4)nc(-c4ccc5c(ccc6c7ccccc7oc56)c4)n3)cc2)cc1 ; c1ccc(-c2ccc(-c3nc(-c4ccccc4)nc(-c4ccc5ccc6c7ccccc7oc6c5c4)n3)cc2)cc1]

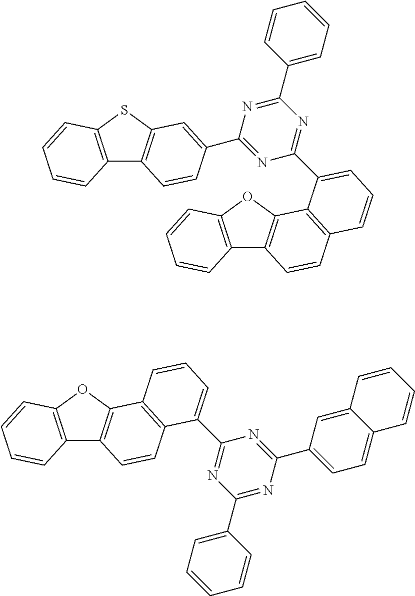 [SMILES: c1ccc(-c2nc(-c3ccc4c(c3)sc3ccccc34)nc(-c3cccc4ccc5c6ccccc6oc5c34)n2)cc1 ; c1ccc(-c2nc(-c3ccc4ccccc4c3)nc(-c3cccc4c3ccc3c5ccccc5oc43)n2)cc1]

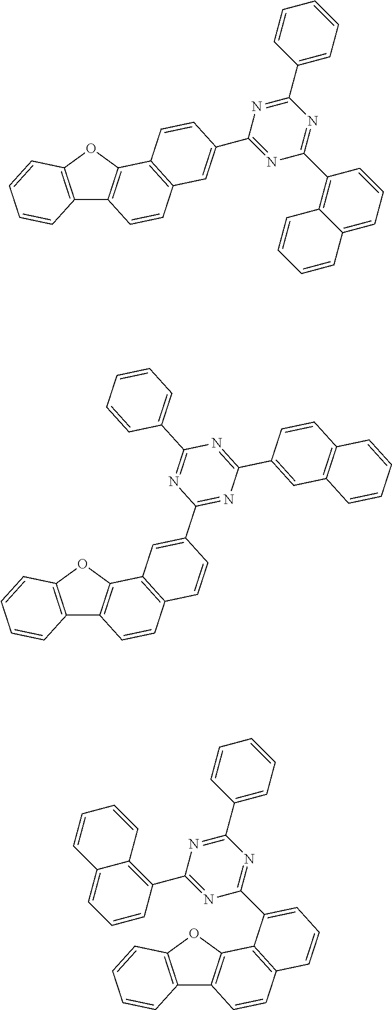 [SMILES: c1ccc(-c2nc(-c3ccc4c(ccc5c6ccccc6oc45)c3)nc(-c3cccc4ccccc34)n2)cc1 ; c1ccc(-c2nc(-c3ccc4ccccc4c3)nc(-c3ccc4ccc5c6ccccc6oc5c4c3)n2)cc1 ; c1ccc(-c2nc(-c3cccc4ccccc34)nc(-c3cccc4ccc5c6ccccc6oc5c34)n2)cc1]

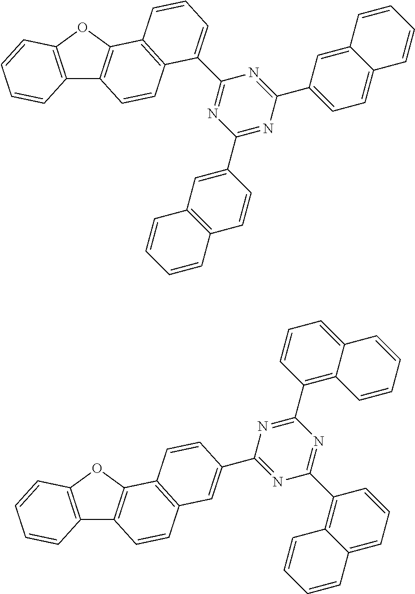 [SMILES: c1ccc2cc(-c3nc(-c4ccc5ccccc5c4)nc(-c4cccc5c4ccc4c6ccccc6oc54)n3)ccc2c1 ; c1ccc2c(-c3nc(-c4ccc5c(ccc6c7ccccc7oc56)c4)nc(-c4cccc5ccccc45)n3)cccc2c1]

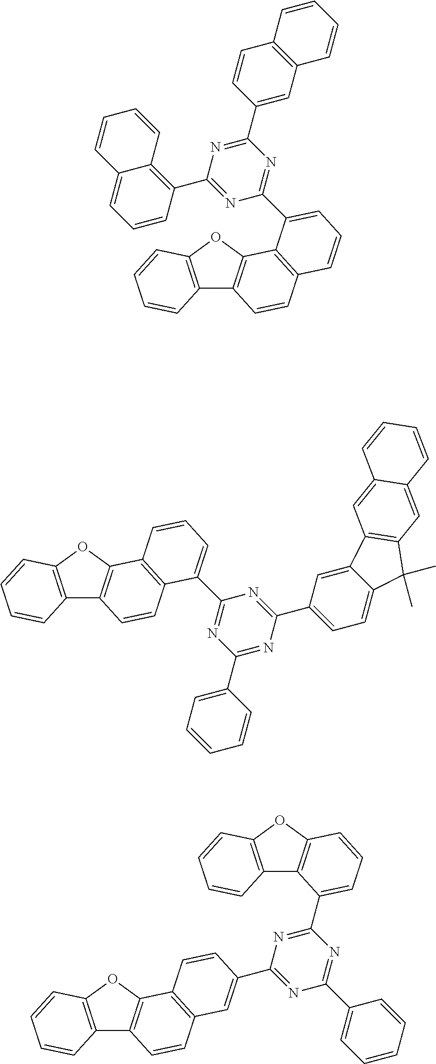 [SMILES: c1ccc2cc(-c3nc(-c4cccc5ccccc45)nc(-c4cccc5ccc6c7ccccc7oc6c45)n3)ccc2c1 ; CC1(C)c2ccc(-c3nc(-c4ccccc4)nc(-c4cccc5c4ccc4c6ccccc6oc54)n3)cc2-c2cc3ccccc3cc21 ; c1ccc(-c2nc(-c3ccc4c(ccc5c6ccccc6oc45)c3)nc(-c3cccc4oc5ccccc5c34)n2)cc1]

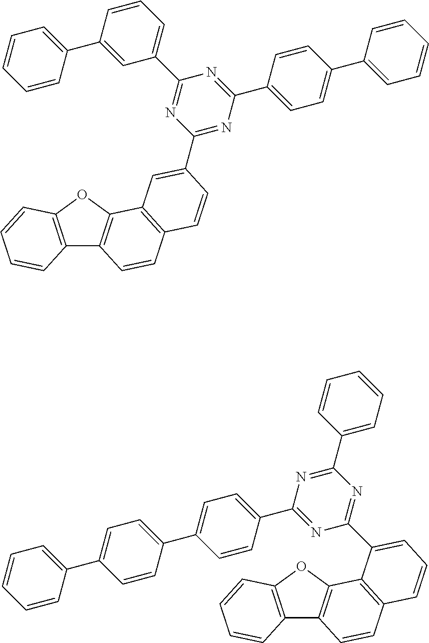 [SMILES: c1ccc(-c2ccc(-c3nc(-c4cccc(-c5ccccc5)c4)nc(-c4ccc5ccc6c7ccccc7oc6c5c4)n3)cc2)cc1 ; c1ccc(-c2ccc(-c3ccc(-c4nc(-c5ccccc5)nc(-c5cccc6ccc7c8ccccc8oc7c56)n4)cc3)cc2)cc1]

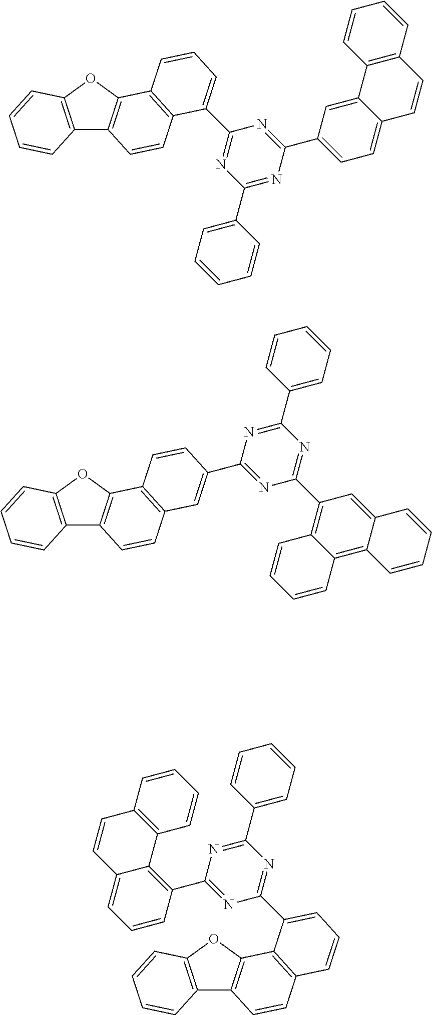 [SMILES: c1ccc(-c2nc(-c3ccc4ccc5ccccc5c4c3)nc(-c3cccc4c3ccc3c5ccccc5oc43)n2)cc1 ; c1ccc(-c2nc(-c3ccc4c(ccc5c6ccccc6oc45)c3)nc(-c3cc4ccccc4c4ccccc34)n2)cc1 ; c1ccc(-c2nc(-c3cccc4ccc5ccccc5c34)nc(-c3cccc4ccc5c6ccccc6oc5c34)n2)cc1]

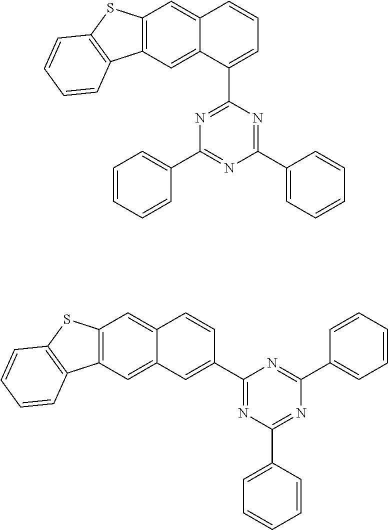 [SMILES: c1ccc(-c2nc(-c3ccccc3)nc(-c3cccc4cc5sc6ccccc6c5cc34)n2)cc1 ; c1ccc(-c2nc(-c3ccccc3)nc(-c3ccc4cc5sc6ccccc6c5cc4c3)n2)cc1]

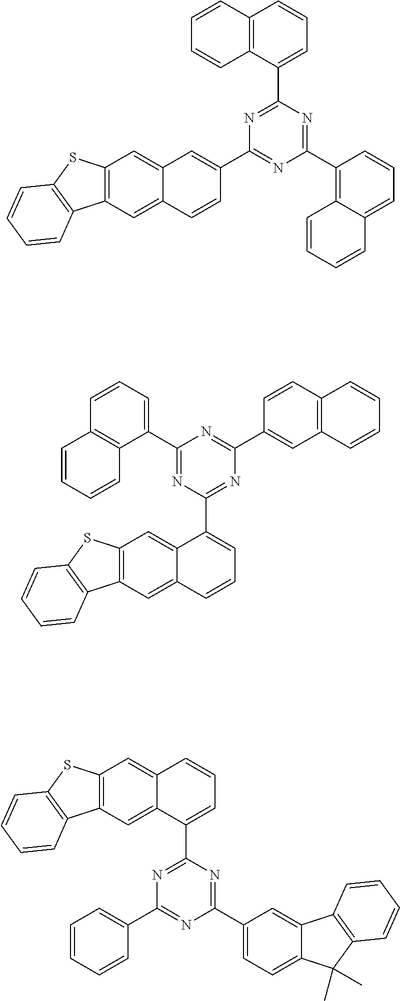 [SMILES: c1ccc2c(-c3nc(-c4ccc5cc6c(cc5c4)sc4ccccc46)nc(-c4cccc5ccccc45)n3)cccc2c1 ; c1ccc2cc(-c3nc(-c4cccc5ccccc45)nc(-c4cccc5cc6c(cc45)sc4ccccc46)n3)ccc2c1 ; CC1(C)c2ccccc2-c2cc(-c3nc(-c4ccccc4)nc(-c4cccc5cc6sc7ccccc7c6cc45)n3)ccc21]

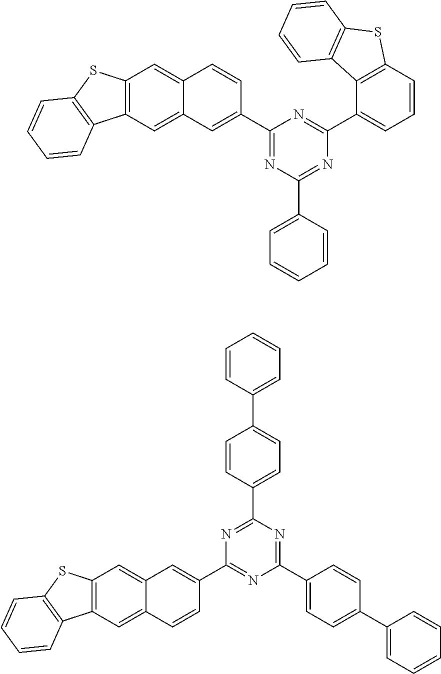 [SMILES: c1ccc(-c2nc(-c3ccc4cc5sc6ccccc6c5cc4c3)nc(-c3cccc4sc5ccccc5c34)n2)cc1 ; c1ccc(-c2ccc(-c3nc(-c4ccc(-c5ccccc5)cc4)nc(-c4ccc5cc6c(cc5c4)sc4ccccc46)n3)cc2)cc1]

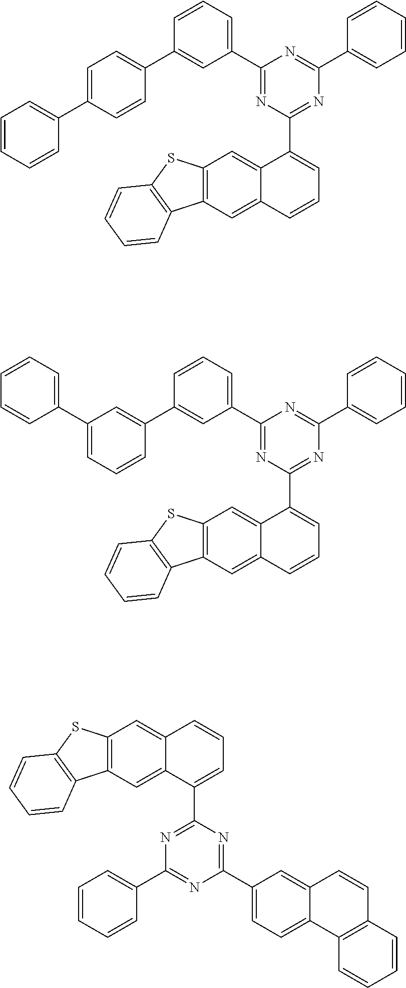 [SMILES: c1ccc(-c2ccc(-c3cccc(-c4nc(-c5ccccc5)nc(-c5cccc6cc7c(cc56)sc5ccccc57)n4)c3)cc2)cc1 ; c1ccc(-c2cccc(-c3cccc(-c4nc(-c5ccccc5)nc(-c5cccc6cc7c(cc56)sc5ccccc57)n4)c3)c2)cc1 ; c1ccc(-c2nc(-c3ccc4c(ccc5ccccc54)c3)nc(-c3cccc4cc5sc6ccccc6c5cc34)n2)cc1]

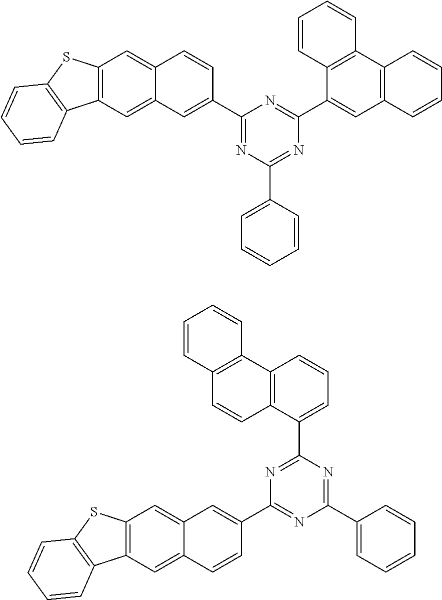 [SMILES: c1ccc(-c2nc(-c3ccc4cc5sc6ccccc6c5cc4c3)nc(-c3cc4ccccc4c4ccccc34)n2)cc1 ; c1ccc(-c2nc(-c3ccc4cc5c(cc4c3)sc3ccccc35)nc(-c3cccc4c3ccc3ccccc34)n2)cc1]

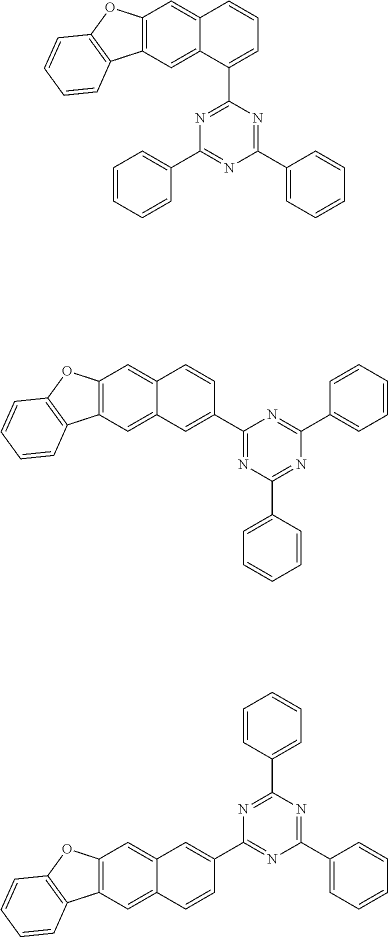 [SMILES: c1ccc(-c2nc(-c3ccccc3)nc(-c3cccc4cc5oc6ccccc6c5cc34)n2)cc1 ; c1ccc(-c2nc(-c3ccccc3)nc(-c3ccc4cc5oc6ccccc6c5cc4c3)n2)cc1 ; c1ccc(-c2nc(-c3ccccc3)nc(-c3ccc4cc5c(cc4c3)oc3ccccc35)n2)cc1]

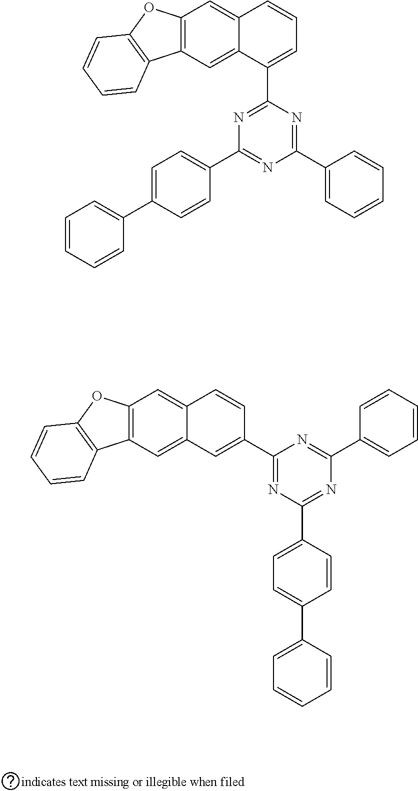 [SMILES: c1ccc(-c2ccc(-c3nc(-c4ccccc4)nc(-c4cccc5cc6oc7ccccc7c6cc45)n3)cc2)cc1 ; c1ccc(-c2ccc(-c3nc(-c4ccccc4)nc(-c4ccc5cc6oc7ccccc7c6cc5c4)n3)cc2)cc1]

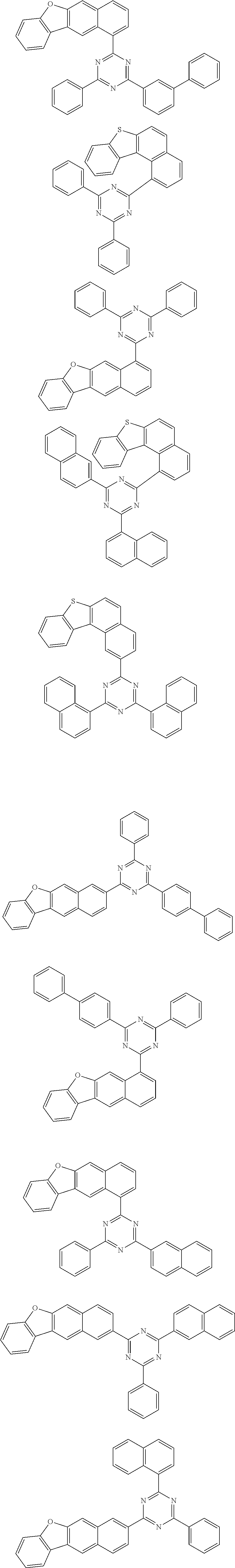

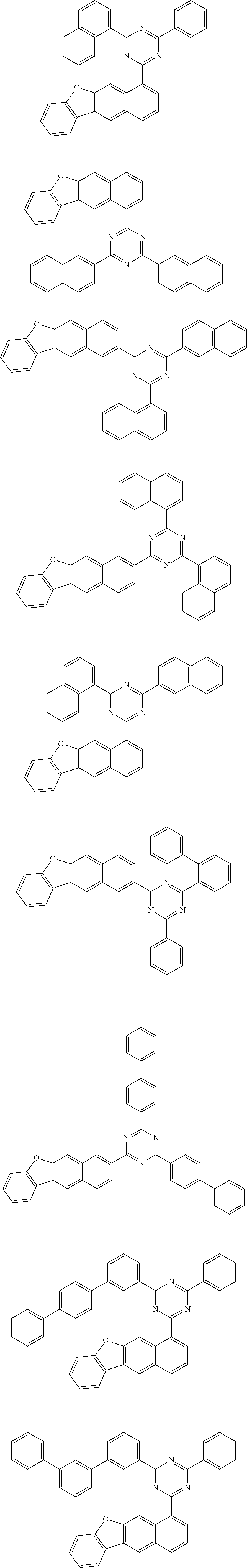

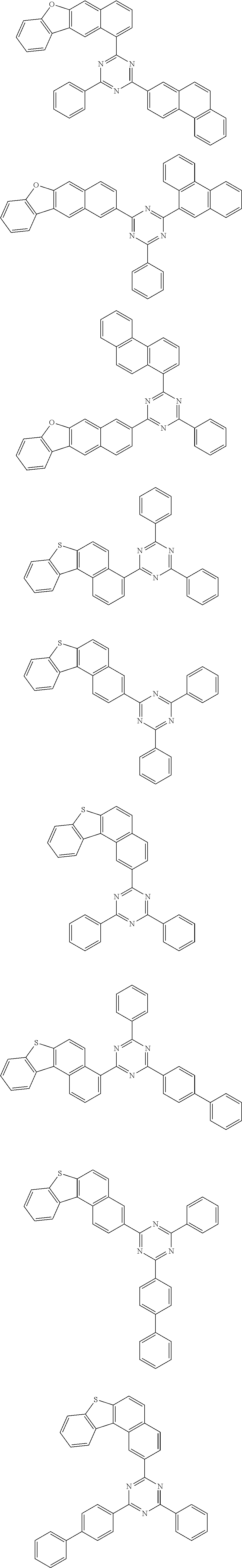

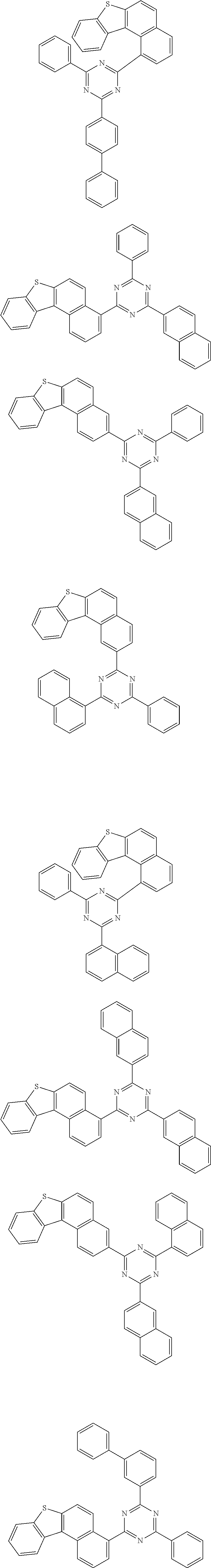

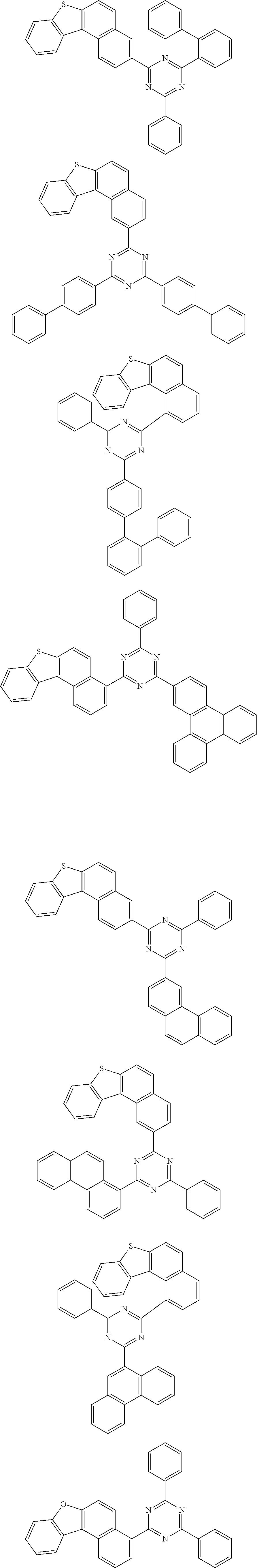

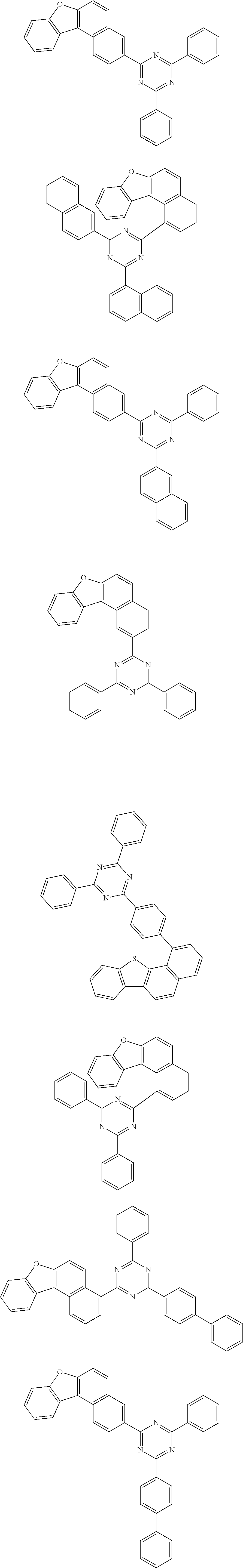

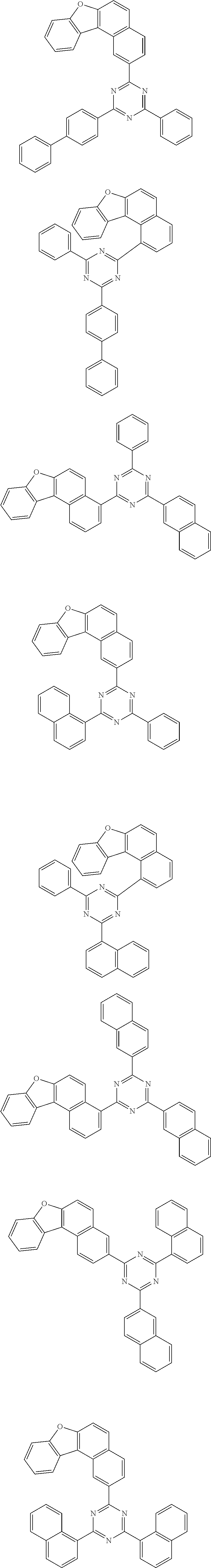

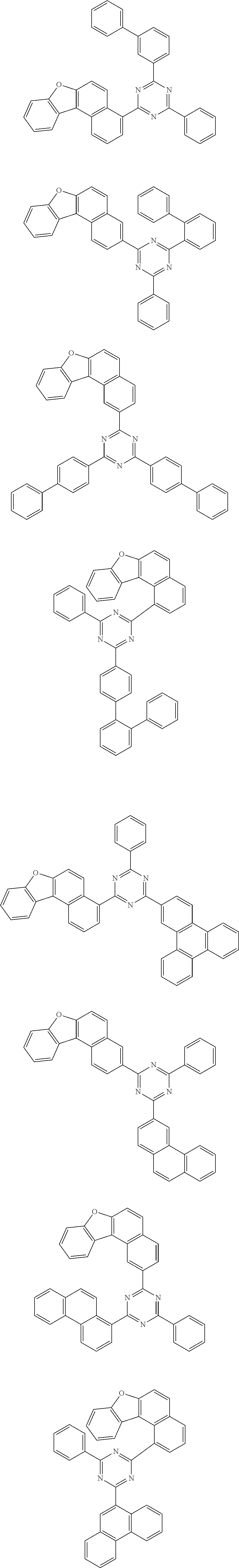

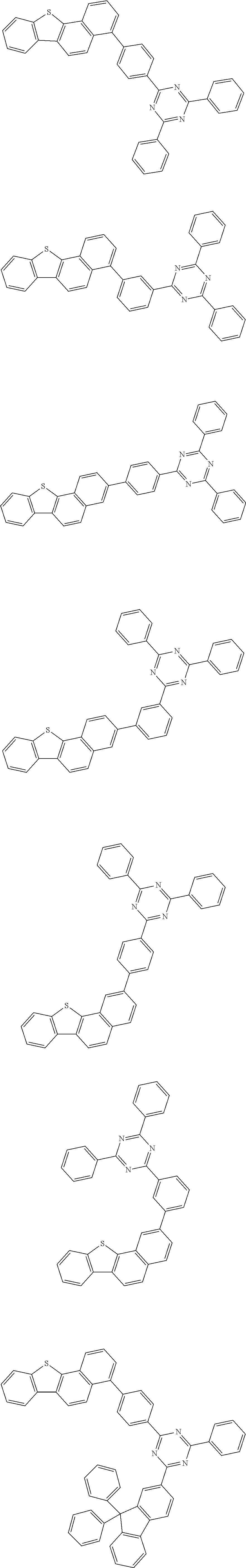

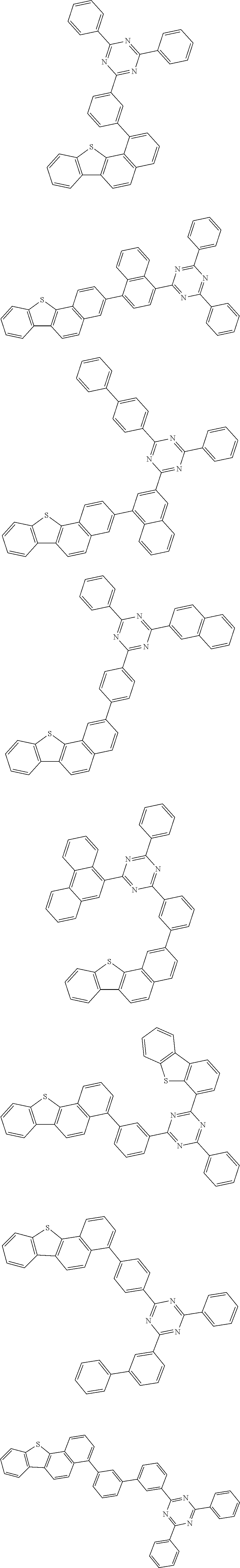

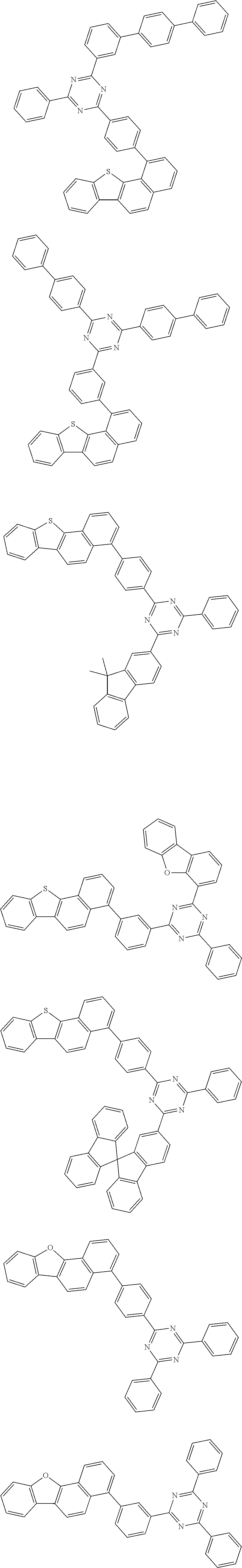

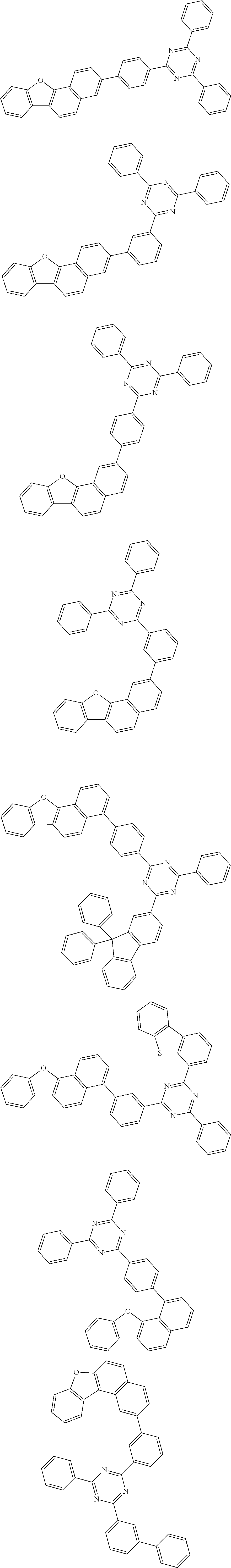

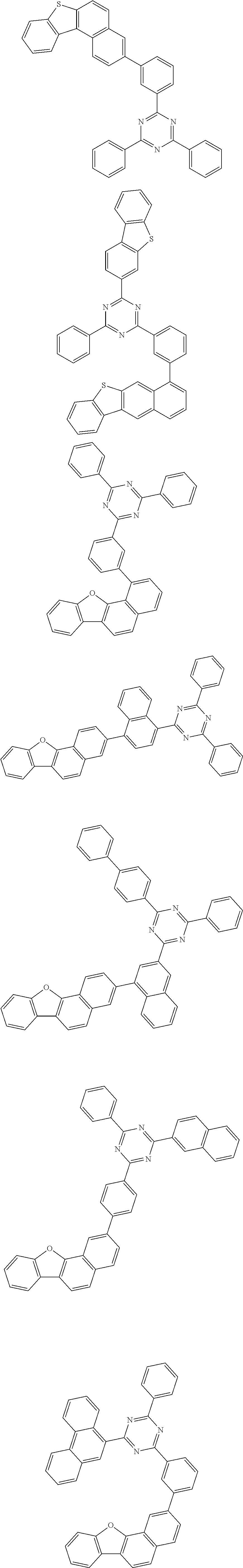

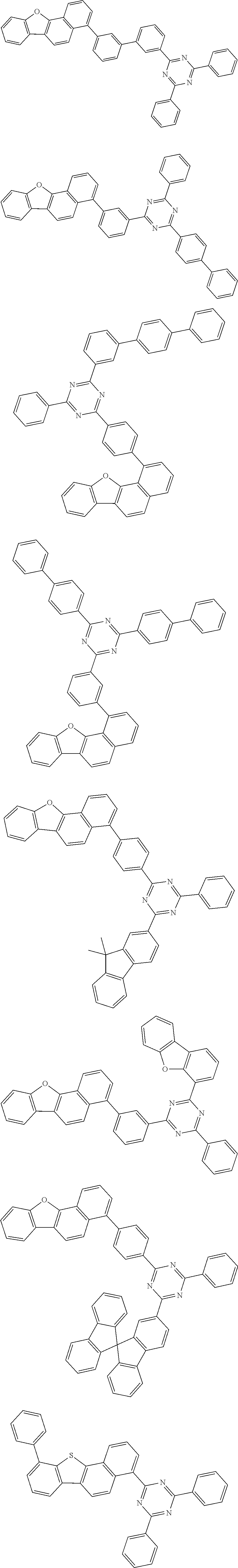

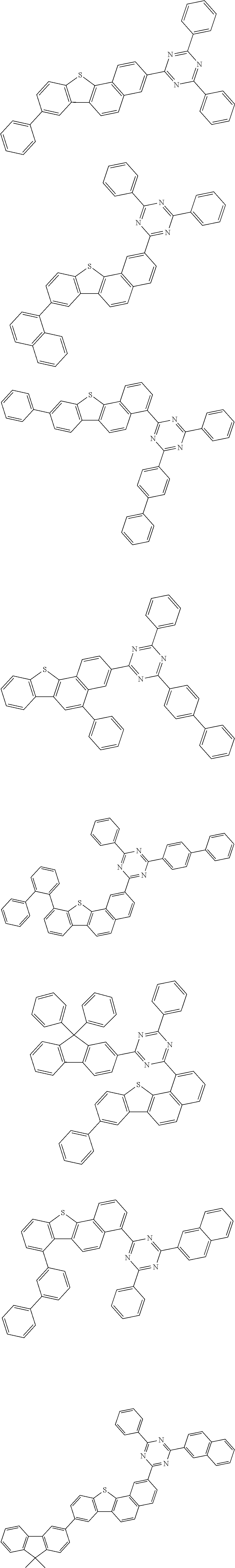

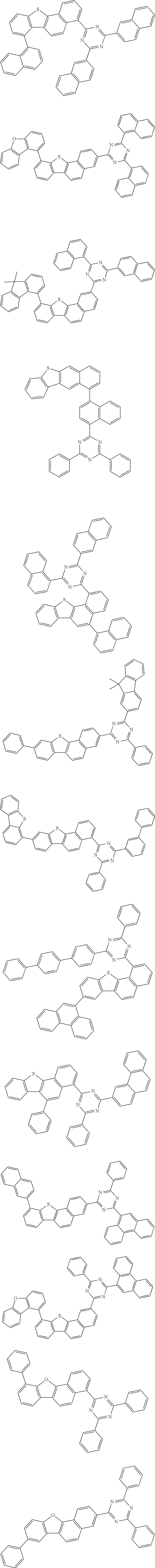

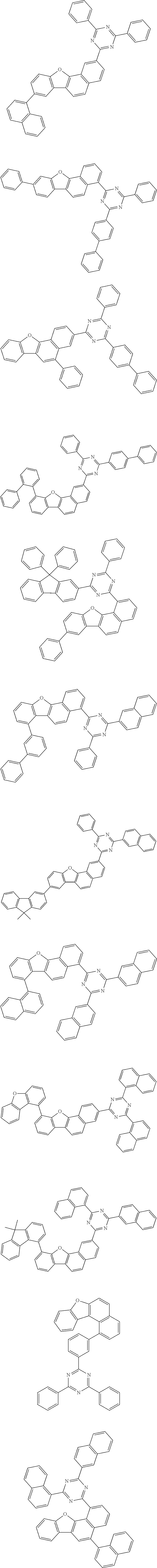

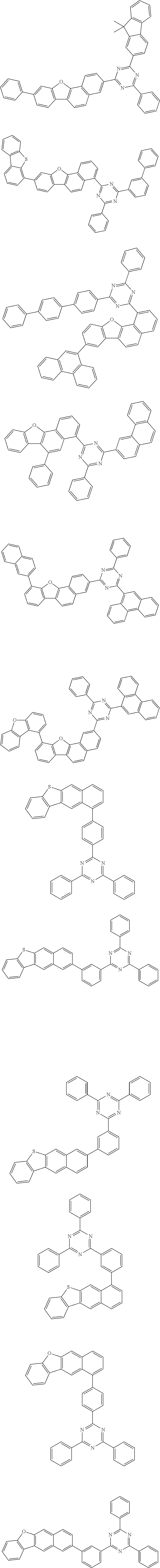

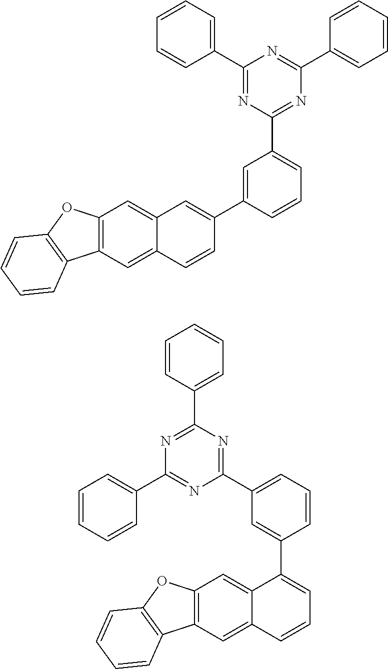 [SMILES: c1ccc(-c2nc(-c3ccccc3)nc(-c3cccc(-c4ccc5cc6c(cc5c4)oc4ccccc46)c3)n2)cc1 ; c1ccc(-c2nc(-c3ccccc3)nc(-c3cccc(-c4cccc5cc6c(cc45)oc4ccccc46)c3)n2)cc1]

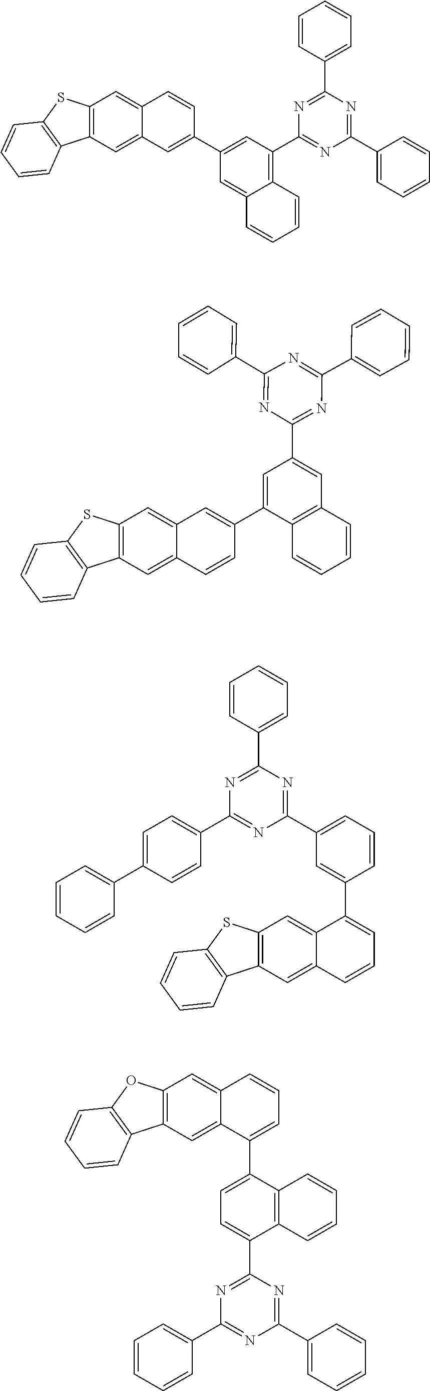 [SMILES: c1ccc(-c2nc(-c3ccccc3)nc(-c3cc(-c4ccc5cc6sc7ccccc7c6cc5c4)cc4ccccc34)n2)cc1 ; c1ccc(-c2nc(-c3ccccc3)nc(-c3cc(-c4ccc5cc6c(cc5c4)sc4ccccc46)c4ccccc4c3)n2)cc1 ; c1ccc(-c2ccc(-c3nc(-c4ccccc4)nc(-c4cccc(-c5cccc6cc7c(cc56)sc5ccccc57)c4)n3)cc2)cc1 ; c1ccc(-c2nc(-c3ccccc3)nc(-c3ccc(-c4cccc5cc6oc7ccccc7c6cc45)c4ccccc34)n2)cc1]

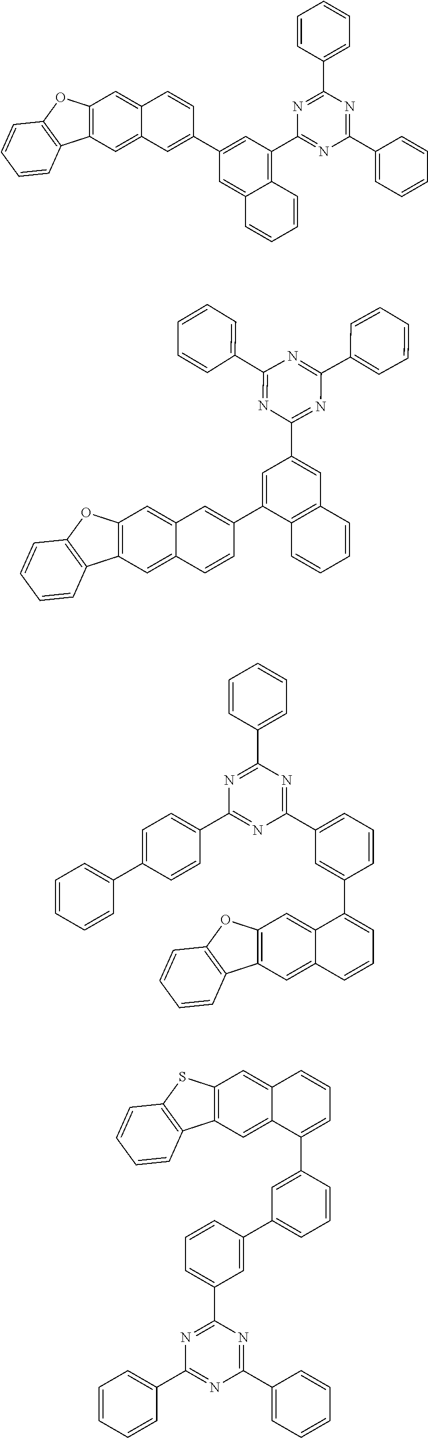 [SMILES: c1ccc(-c2nc(-c3ccccc3)nc(-c3cc(-c4ccc5cc6oc7ccccc7c6cc5c4)cc4ccccc34)n2)cc1 ; c1ccc(-c2nc(-c3ccccc3)nc(-c3cc(-c4ccc5cc6c(cc5c4)oc4ccccc46)c4ccccc4c3)n2)cc1 ; c1ccc(-c2ccc(-c3nc(-c4ccccc4)nc(-c4cccc(-c5cccc6cc7c(cc56)oc5ccccc57)c4)n3)cc2)cc1 ; c1ccc(-c2nc(-c3ccccc3)nc(-c3cccc(-c4cccc(-c5cccc6cc7sc8ccccc8c7cc56)c4)c3)n2)cc1]

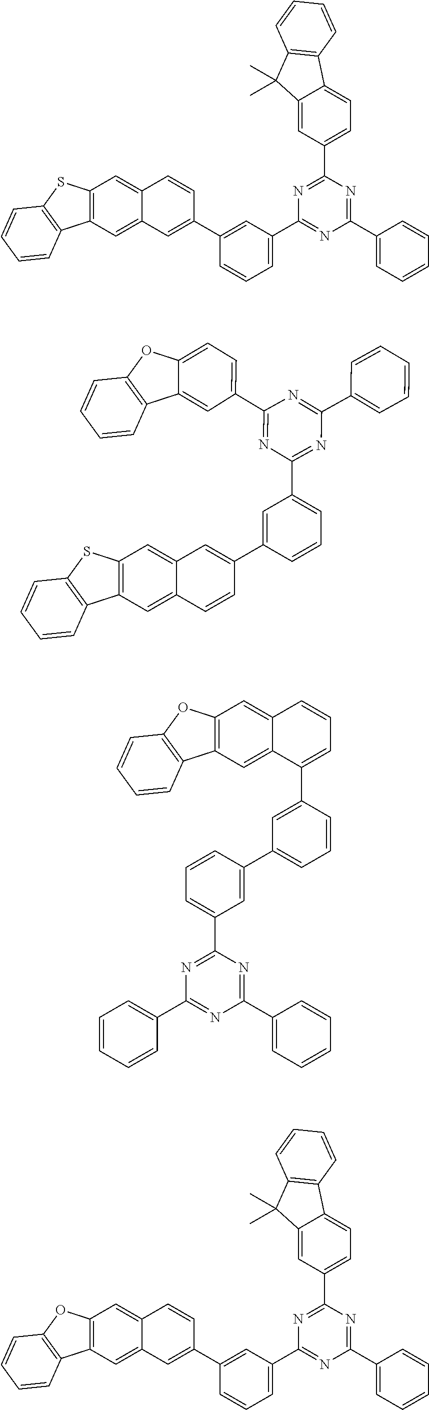 [SMILES: CC1(C)c2ccccc2-c2ccc(-c3nc(-c4ccccc4)nc(-c4cccc(-c5ccc6cc7sc8ccccc8c7cc6c5)c4)n3)cc21 ; c1ccc(-c2nc(-c3cccc(-c4ccc5cc6c(cc5c4)sc4ccccc46)c3)nc(-c3ccc4oc5ccccc5c4c3)n2)cc1 ; c1ccc(-c2nc(-c3ccccc3)nc(-c3cccc(-c4cccc(-c5cccc6cc7oc8ccccc8c7cc56)c4)c3)n2)cc1 ; CC1(C)c2ccccc2-c2ccc(-c3nc(-c4ccccc4)nc(-c4cccc(-c5ccc6cc7oc8ccccc8c7cc6c5)c4)n3)cc21]

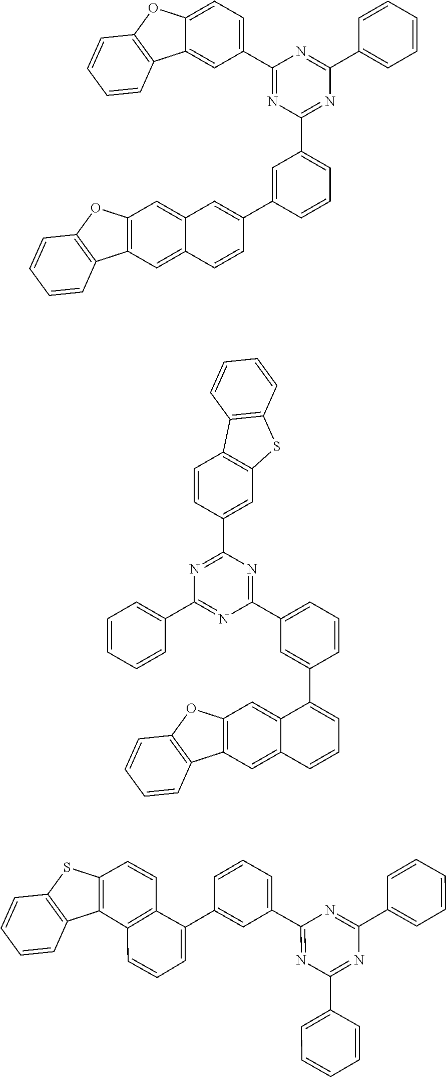 [SMILES: c1ccc(-c2nc(-c3cccc(-c4ccc5cc6c(cc5c4)oc4ccccc46)c3)nc(-c3ccc4oc5ccccc5c4c3)n2)cc1 ; c1ccc(-c2nc(-c3cccc(-c4cccc5cc6c(cc45)oc4ccccc46)c3)nc(-c3ccc4c(c3)sc3ccccc34)n2)cc1 ; c1ccc(-c2nc(-c3ccccc3)nc(-c3cccc(-c4cccc5c4ccc4sc6ccccc6c45)c3)n2)cc1]

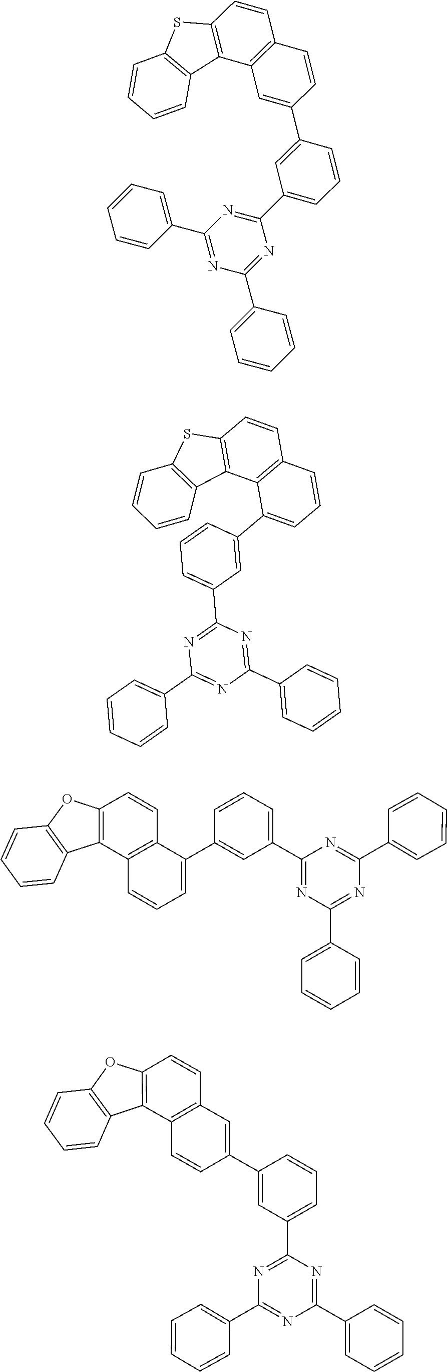 [SMILES: c1ccc(-c2nc(-c3ccccc3)nc(-c3cccc(-c4ccc5ccc6sc7ccccc7c6c5c4)c3)n2)cc1 ; c1ccc(-c2nc(-c3ccccc3)nc(-c3cccc(-c4cccc5ccc6sc7ccccc7c6c45)c3)n2)cc1 ; c1ccc(-c2nc(-c3ccccc3)nc(-c3cccc(-c4cccc5c4ccc4oc6ccccc6c45)c3)n2)cc1 ; c1ccc(-c2nc(-c3ccccc3)nc(-c3cccc(-c4ccc5c(ccc6oc7ccccc7c65)c4)c3)n2)cc1]

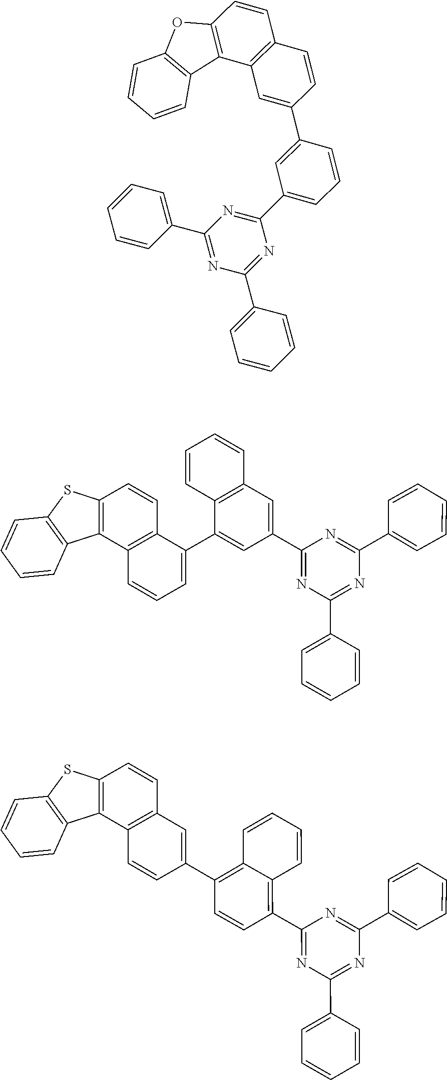 [SMILES: c1ccc(-c2nc(-c3ccccc3)nc(-c3cccc(-c4ccc5ccc6oc7ccccc7c6c5c4)c3)n2)cc1 ; c1ccc(-c2nc(-c3ccccc3)nc(-c3cc(-c4cccc5c4ccc4sc6ccccc6c45)c4ccccc4c3)n2)cc1 ; c1ccc(-c2nc(-c3ccccc3)nc(-c3ccc(-c4ccc5c(ccc6sc7ccccc7c65)c4)c4ccccc34)n2)cc1]

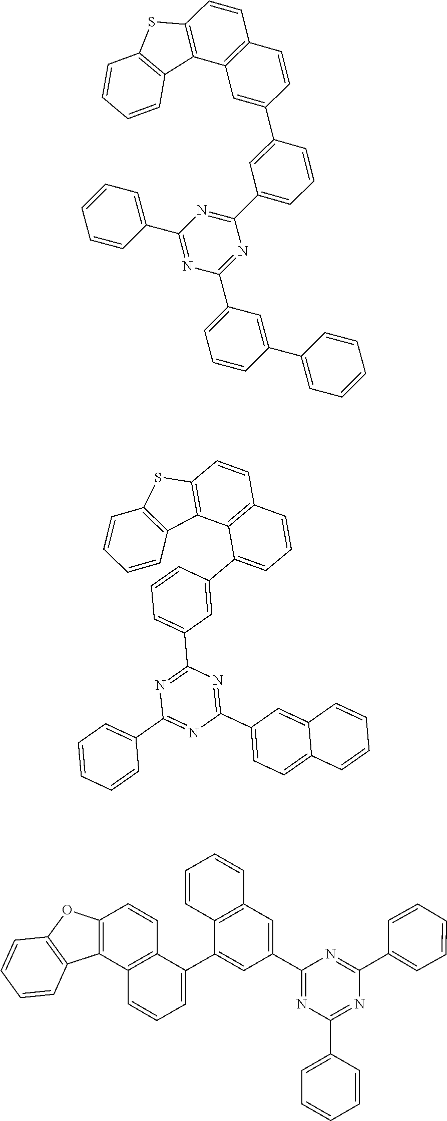 [SMILES: c1ccc(-c2cccc(-c3nc(-c4ccccc4)nc(-c4cccc(-c5ccc6ccc7sc8ccccc8c7c6c5)c4)n3)c2)cc1 ; c1ccc(-c2nc(-c3cccc(-c4cccc5ccc6sc7ccccc7c6c45)c3)nc(-c3ccc4ccccc4c3)n2)cc1 ; c1ccc(-c2nc(-c3ccccc3)nc(-c3cc(-c4cccc5c4ccc4oc6ccccc6c45)c4ccccc4c3)n2)cc1]

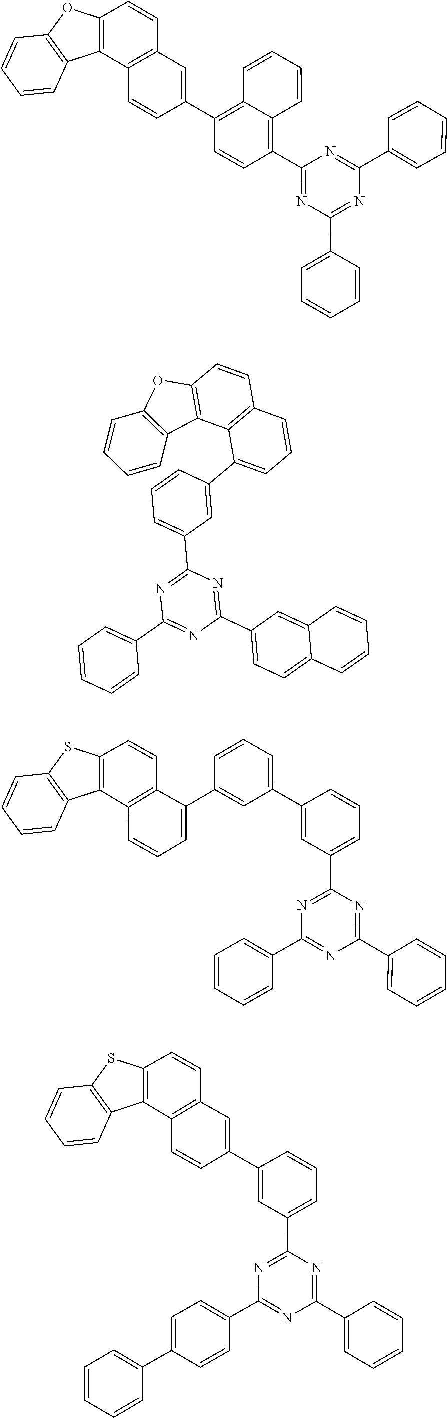 [SMILES: c1ccc(-c2nc(-c3ccccc3)nc(-c3ccc(-c4ccc5c(ccc6oc7ccccc7c65)c4)c4ccccc34)n2)cc1 ; c1ccc(-c2nc(-c3cccc(-c4cccc5ccc6oc7ccccc7c6c45)c3)nc(-c3ccc4ccccc4c3)n2)cc1 ; c1ccc(-c2nc(-c3ccccc3)nc(-c3cccc(-c4cccc(-c5cccc6c5ccc5sc7ccccc7c56)c4)c3)n2)cc1 ; c1ccc(-c2ccc(-c3nc(-c4ccccc4)nc(-c4cccc(-c5ccc6c(ccc7sc8ccccc8c76)c5)c4)n3)cc2)cc1]

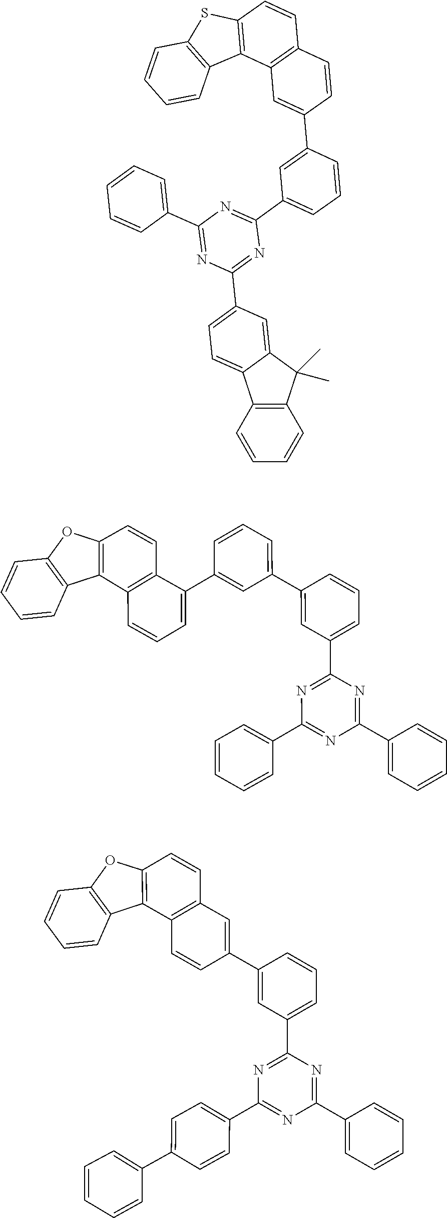 [SMILES: CC1(C)c2ccccc2-c2ccc(-c3nc(-c4ccccc4)nc(-c4cccc(-c5ccc6ccc7sc8ccccc8c7c6c5)c4)n3)cc21 ; c1ccc(-c2nc(-c3ccccc3)nc(-c3cccc(-c4cccc(-c5cccc6c5ccc5oc7ccccc7c56)c4)c3)n2)cc1 ; c1ccc(-c2ccc(-c3nc(-c4ccccc4)nc(-c4cccc(-c5ccc6c(ccc7oc8ccccc8c76)c5)c4)n3)cc2)cc1]

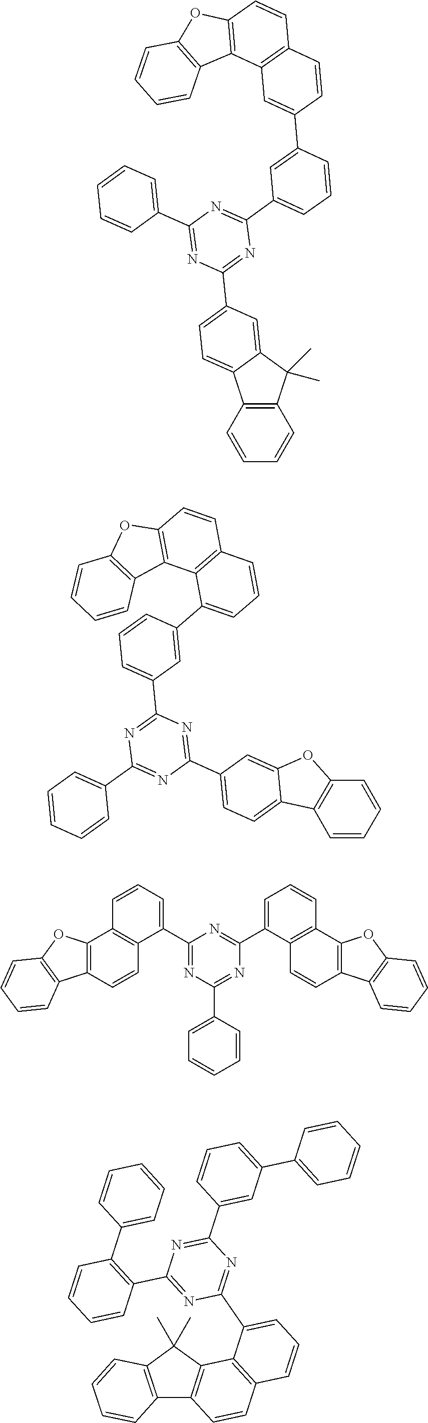 [SMILES: CC1(C)c2ccccc2-c2ccc(-c3nc(-c4ccccc4)nc(-c4cccc(-c5ccc6ccc7oc8ccccc8c7c6c5)c4)n3)cc21 ; c1ccc(-c2nc(-c3cccc(-c4cccc5ccc6oc7ccccc7c6c45)c3)nc(-c3ccc4c(c3)oc3ccccc34)n2)cc1 ; c1ccc(-c2nc(-c3cccc4c3ccc3c5ccccc5oc43)nc(-c3cccc4c3ccc3c5ccccc5oc43)n2)cc1 ; CC1(C)c2ccccc2-c2ccc3cccc(-c4nc(-c5cccc(-c6ccccc6)c5)nc(-c5ccccc5-c5ccccc5)n4)c3c21]

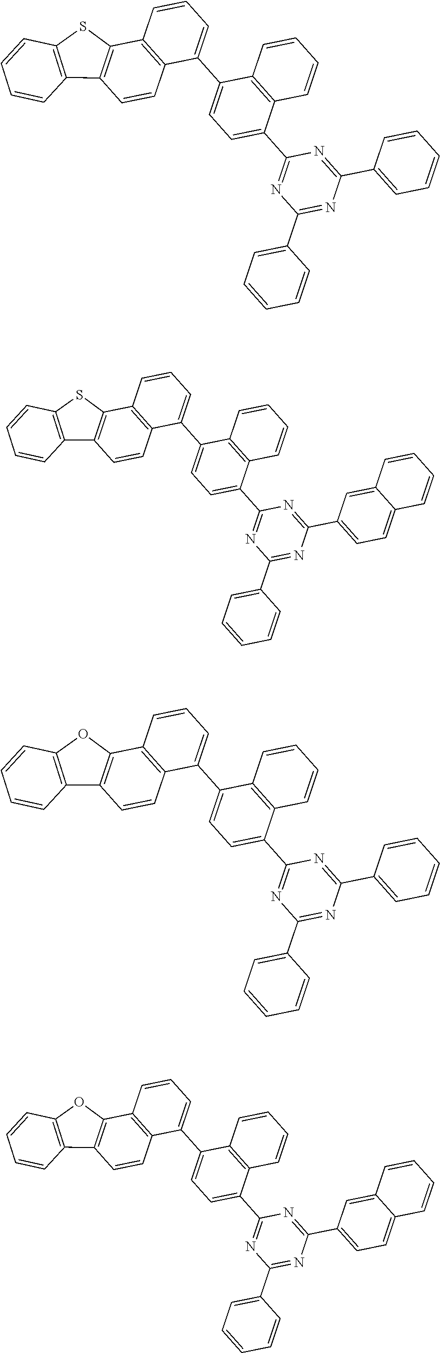 [SMILES: c1ccc(-c2nc(-c3ccccc3)nc(-c3ccc(-c4cccc5c4ccc4c6ccccc6sc54)c4ccccc34)n2)cc1 ; c1ccc(-c2nc(-c3ccc4ccccc4c3)nc(-c3ccc(-c4cccc5c4ccc4c6ccccc6sc54)c4ccccc34)n2)cc1 ; c1ccc(-c2nc(-c3ccccc3)nc(-c3ccc(-c4cccc5c4ccc4c6ccccc6oc54)c4ccccc34)n2)cc1 ; c1ccc(-c2nc(-c3ccc4ccccc4c3)nc(-c3ccc(-c4cccc5c4ccc4c6ccccc6oc54)c4ccccc34)n2)cc1]

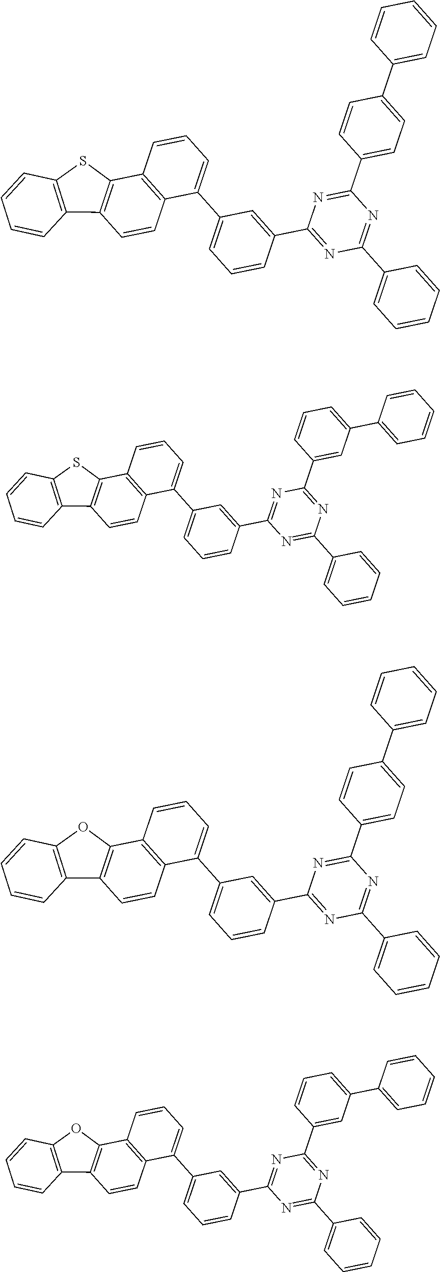 [SMILES: c1ccc(-c2ccc(-c3nc(-c4ccccc4)nc(-c4cccc(-c5cccc6c5ccc5c7ccccc7sc65)c4)n3)cc2)cc1 ; c1ccc(-c2cccc(-c3nc(-c4ccccc4)nc(-c4cccc(-c5cccc6c5ccc5c7ccccc7sc65)c4)n3)c2)cc1 ; c1ccc(-c2ccc(-c3nc(-c4ccccc4)nc(-c4cccc(-c5cccc6c5ccc5c7ccccc7oc65)c4)n3)cc2)cc1 ; c1ccc(-c2cccc(-c3nc(-c4ccccc4)nc(-c4cccc(-c5cccc6c5ccc5c7ccccc7oc65)c4)n3)c2)cc1]

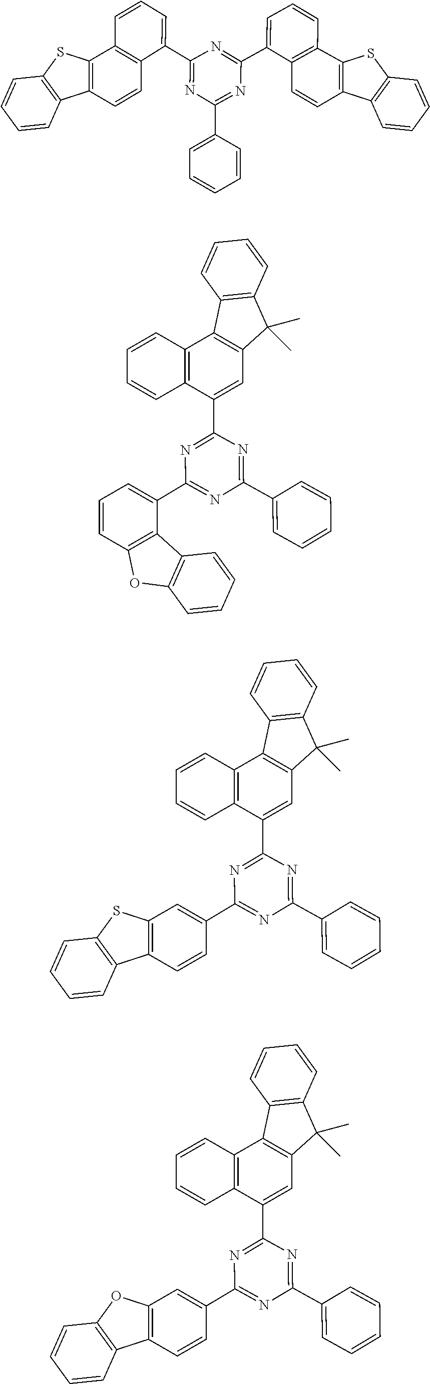 [SMILES: c1ccc(-c2nc(-c3cccc4c3ccc3c5ccccc5sc43)nc(-c3cccc4c3ccc3c5ccccc5sc43)n2)cc1 ; CC1(C)c2ccccc2-c2c1cc(-c1nc(-c3ccccc3)nc(-c3cccc4oc5ccccc5c34)n1)c1ccccc21 ; CC1(C)c2ccccc2-c2c1cc(-c1nc(-c3ccccc3)nc(-c3ccc4c(c3)sc3ccccc34)n1)c1ccccc21 ; CC1(C)c2ccccc2-c2c1cc(-c1nc(-c3ccccc3)nc(-c3ccc4c(c3)oc3ccccc34)n1)c1ccccc21]

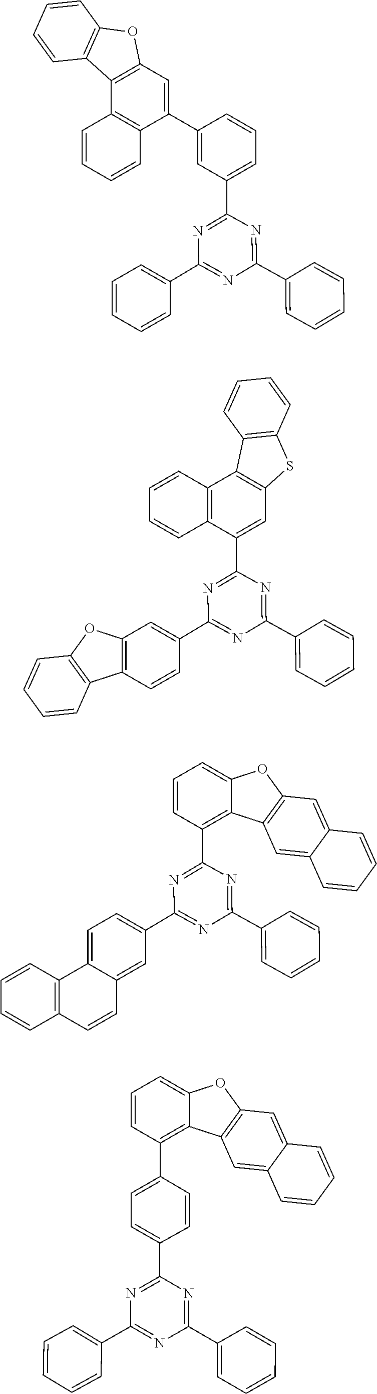 [SMILES: c1ccc(-c2nc(-c3ccccc3)nc(-c3cccc(-c4cc5oc6ccccc6c5c5ccccc45)c3)n2)cc1 ; c1ccc(-c2nc(-c3ccc4c(c3)oc3ccccc34)nc(-c3cc4sc5ccccc5c4c4ccccc34)n2)cc1 ; c1ccc(-c2nc(-c3ccc4c(ccc5ccccc54)c3)nc(-c3cccc4oc5cc6ccccc6cc5c34)n2)cc1 ; c1ccc(-c2nc(-c3ccccc3)nc(-c3ccc(-c4cccc5oc6cc7ccccc7cc6c45)cc3)n2)cc1]

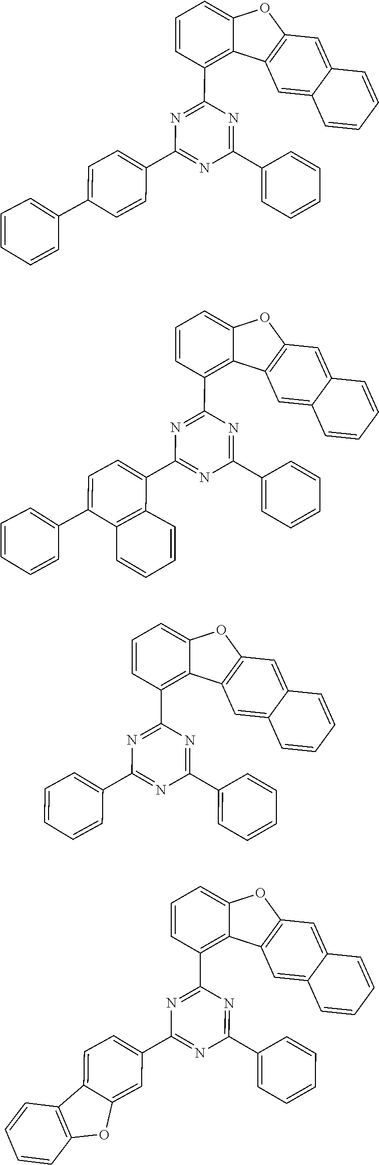 [SMILES: c1ccc(-c2ccc(-c3nc(-c4ccccc4)nc(-c4cccc5oc6cc7ccccc7cc6c45)n3)cc2)cc1 ; c1ccc(-c2nc(-c3ccc(-c4ccccc4)c4ccccc34)nc(-c3cccc4oc5cc6ccccc6cc5c34)n2)cc1 ; c1ccc(-c2nc(-c3ccccc3)nc(-c3cccc4oc5cc6ccccc6cc5c34)n2)cc1 ; c1ccc(-c2nc(-c3ccc4c(c3)oc3ccccc34)nc(-c3cccc4oc5cc6ccccc6cc5c34)n2)cc1]

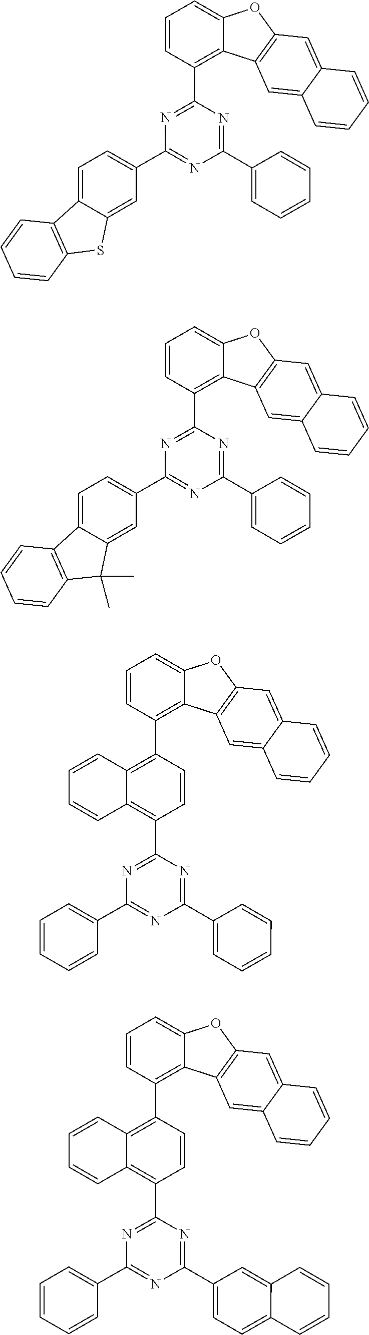 [SMILES: c1ccc(-c2nc(-c3ccc4c(c3)sc3ccccc34)nc(-c3cccc4oc5cc6ccccc6cc5c34)n2)cc1 ; CC1(C)c2ccccc2-c2ccc(-c3nc(-c4ccccc4)nc(-c4cccc5oc6cc7ccccc7cc6c45)n3)cc21 ; c1ccc(-c2nc(-c3ccccc3)nc(-c3ccc(-c4cccc5oc6cc7ccccc7cc6c45)c4ccccc34)n2)cc1 ; c1ccc(-c2nc(-c3ccc4ccccc4c3)nc(-c3ccc(-c4cccc5oc6cc7ccccc7cc6c45)c4ccccc34)n2)cc1]

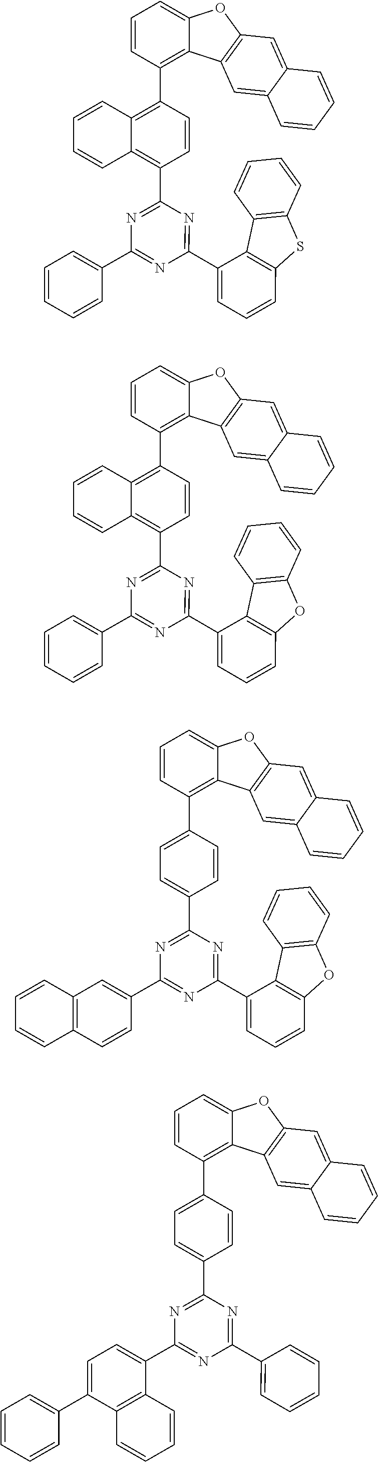 [SMILES: c1ccc(-c2nc(-c3ccc(-c4cccc5oc6cc7ccccc7cc6c45)c4ccccc34)nc(-c3cccc4sc5ccccc5c34)n2)cc1 ; c1ccc(-c2nc(-c3ccc(-c4cccc5oc6cc7ccccc7cc6c45)c4ccccc34)nc(-c3cccc4oc5ccccc5c34)n2)cc1 ; c1ccc2cc(-c3nc(-c4ccc(-c5cccc6oc7cc8ccccc8cc7c56)cc4)nc(-c4cccc5oc6ccccc6c45)n3)ccc2c1 ; c1ccc(-c2nc(-c3ccc(-c4cccc5oc6cc7ccccc7cc6c45)cc3)nc(-c3ccc(-c4ccccc4)c4ccccc34)n2)cc1]

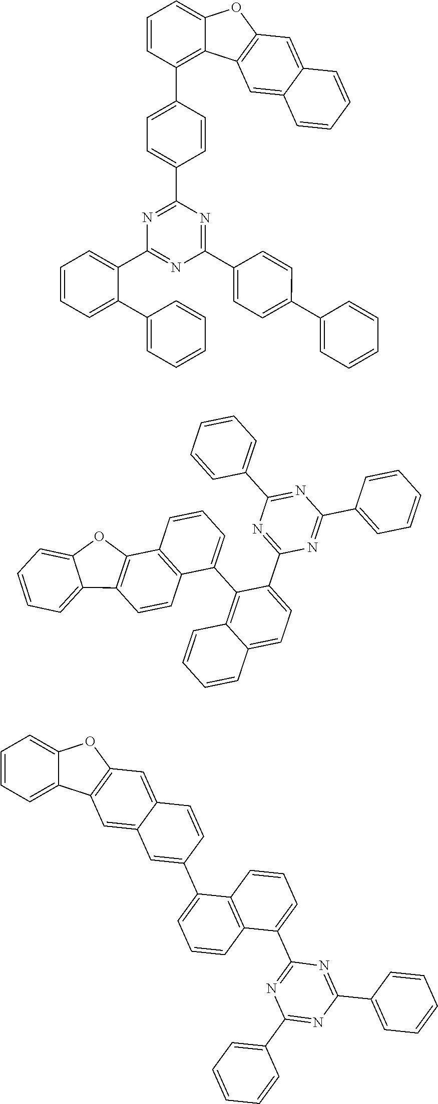 [SMILES: c1ccc(-c2ccc(-c3nc(-c4ccc(-c5cccc6oc7cc8ccccc8cc7c56)cc4)nc(-c4ccccc4-c4ccccc4)n3)cc2)cc1 ; c1ccc(-c2nc(-c3ccccc3)nc(-c3ccc4ccccc4c3-c3cccc4c3ccc3c5ccccc5oc43)n2)cc1 ; c1ccc(-c2nc(-c3ccccc3)nc(-c3cccc4c(-c5ccc6cc7oc8ccccc8c7cc6c5)cccc34)n2)cc1]

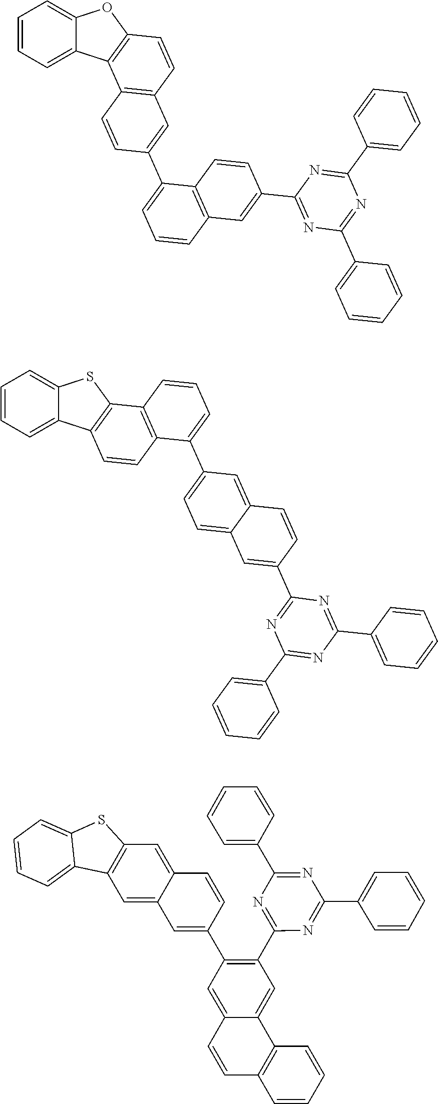 [SMILES: c1ccc(-c2nc(-c3ccccc3)nc(-c3ccc4c(-c5ccc6c(ccc7oc8ccccc8c76)c5)cccc4c3)n2)cc1 ; c1ccc(-c2nc(-c3ccccc3)nc(-c3ccc4cc(-c5cccc6c5ccc5c7ccccc7sc65)ccc4c3)n2)cc1 ; c1ccc(-c2nc(-c3ccccc3)nc(-c3cc4c(ccc5ccccc54)cc3-c3ccc4cc5sc6ccccc6c5cc4c3)n2)cc1]

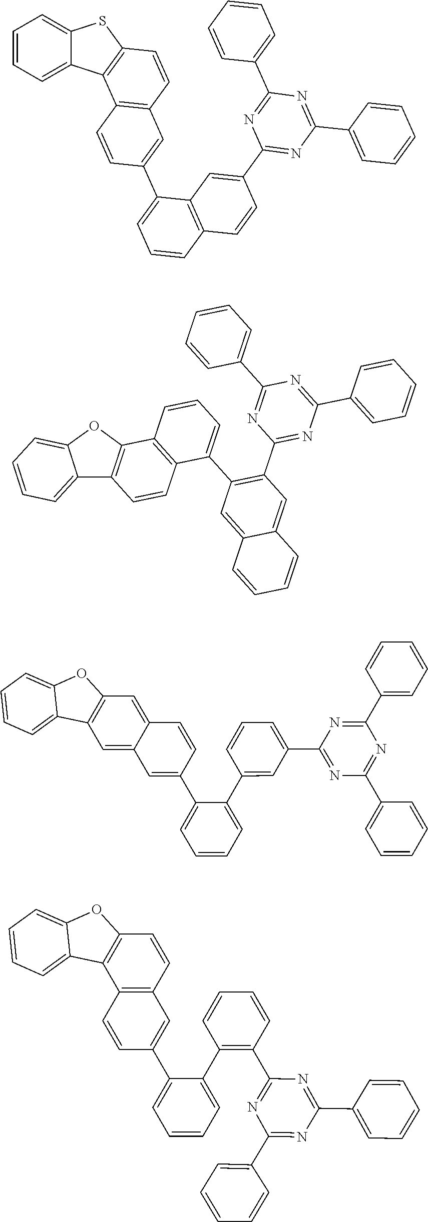 [SMILES: c1ccc(-c2nc(-c3ccccc3)nc(-c3ccc4cccc(-c5ccc6c(ccc7sc8ccccc8c76)c5)c4c3)n2)cc1 ; c1ccc(-c2nc(-c3ccccc3)nc(-c3cc4ccccc4cc3-c3cccc4c3ccc3c5ccccc5oc43)n2)cc1 ; c1ccc(-c2nc(-c3ccccc3)nc(-c3cccc(-c4ccccc4-c4ccc5cc6oc7ccccc7c6cc5c4)c3)n2)cc1 ; c1ccc(-c2nc(-c3ccccc3)nc(-c3ccccc3-c3ccccc3-c3ccc4c(ccc5oc6ccccc6c54)c3)n2)cc1]

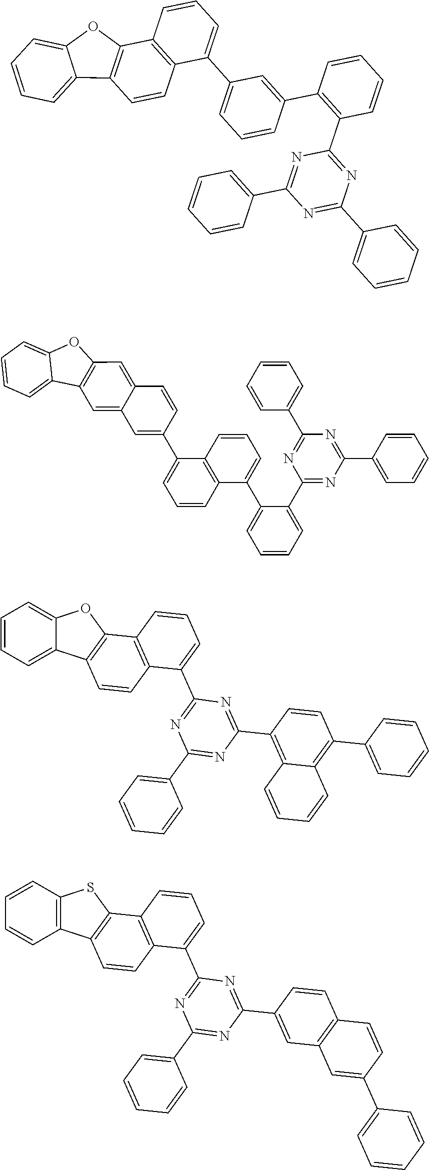 [SMILES: c1ccc(-c2nc(-c3ccccc3)nc(-c3ccccc3-c3cccc(-c4cccc5c4ccc4c6ccccc6oc54)c3)n2)cc1 ; c1ccc(-c2nc(-c3ccccc3)nc(-c3ccccc3-c3cccc4c(-c5ccc6cc7oc8ccccc8c7cc6c5)cccc34)n2)cc1 ; c1ccc(-c2nc(-c3ccc(-c4ccccc4)c4ccccc34)nc(-c3cccc4c3ccc3c5ccccc5oc43)n2)cc1 ; c1ccc(-c2ccc3ccc(-c4nc(-c5ccccc5)nc(-c5cccc6c5ccc5c7ccccc7sc65)n4)cc3c2)cc1]

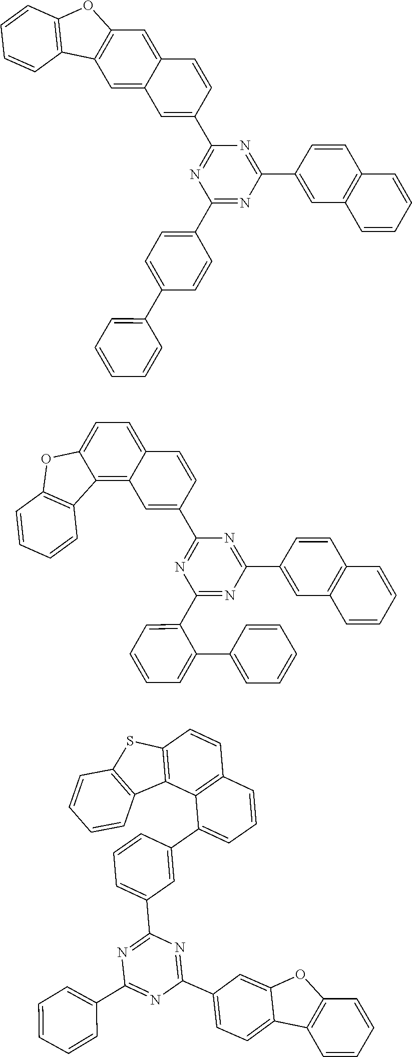 [SMILES: c1ccc(-c2ccc(-c3nc(-c4ccc5ccccc5c4)nc(-c4ccc5cc6oc7ccccc7c6cc5c4)n3)cc2)cc1 ; c1ccc(-c2ccccc2-c2nc(-c3ccc4ccccc4c3)nc(-c3ccc4ccc5oc6ccccc6c5c4c3)n2)cc1 ; c1ccc(-c2nc(-c3cccc(-c4cccc5ccc6sc7ccccc7c6c45)c3)nc(-c3ccc4c(c3)oc3ccccc34)n2)cc1]

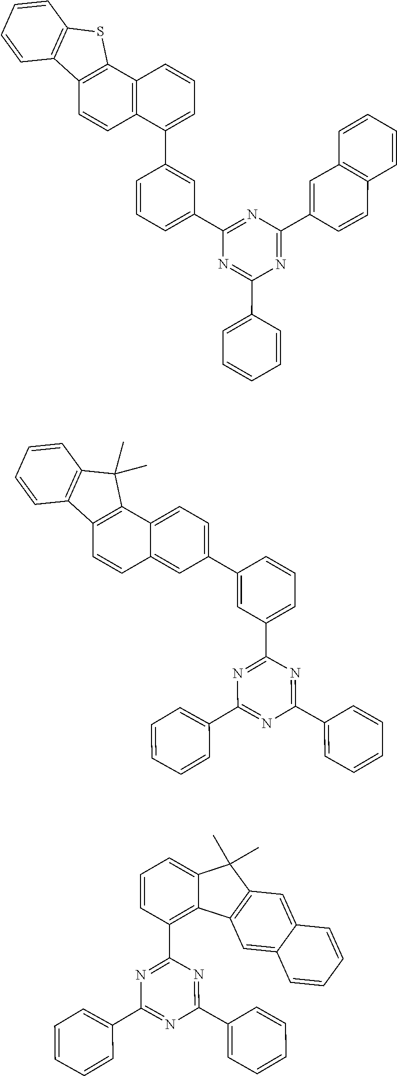 [SMILES: c1ccc(-c2nc(-c3cccc(-c4cccc5c4ccc4c6ccccc6sc54)c3)nc(-c3ccc4ccccc4c3)n2)cc1 ; CC1(C)c2ccccc2-c2ccc3cc(-c4cccc(-c5nc(-c6ccccc6)nc(-c6ccccc6)n5)c4)ccc3c21 ; CC1(C)c2cc3ccccc3cc2-c2c(-c3nc(-c4ccccc4)nc(-c4ccccc4)n3)cccc21]

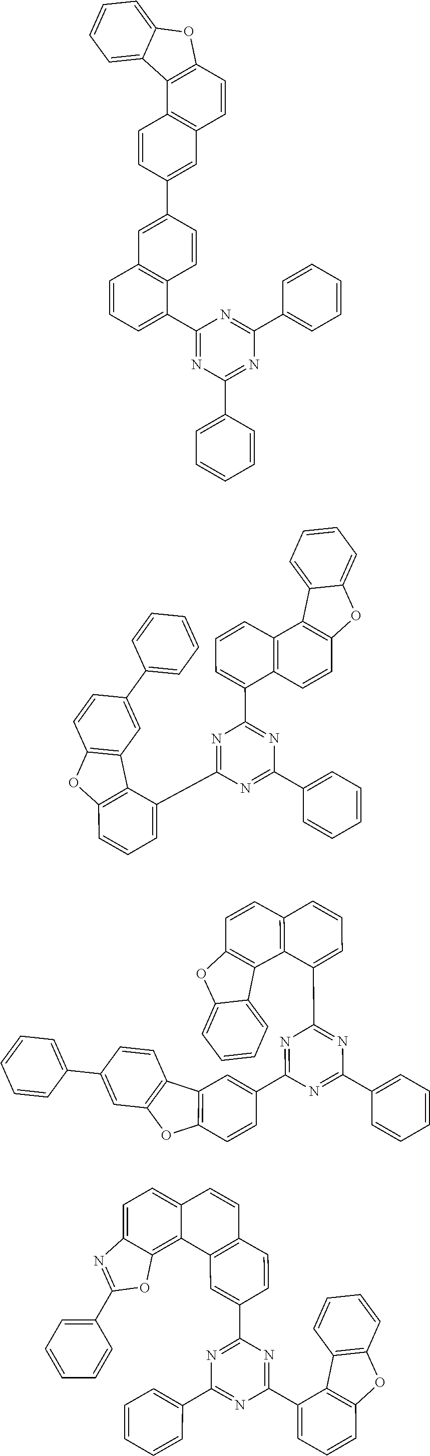 [SMILES: c1ccc(-c2nc(-c3ccccc3)nc(-c3cccc4cc(-c5ccc6c(ccc7oc8ccccc8c76)c5)ccc34)n2)cc1 ; c1ccc(-c2ccc3oc4cccc(-c5nc(-c6ccccc6)nc(-c6cccc7c6ccc6oc8ccccc8c67)n5)c4c3c2)cc1 ; c1ccc(-c2ccc3c(c2)oc2ccc(-c4nc(-c5ccccc5)nc(-c5cccc6ccc7oc8ccccc8c7c56)n4)cc23)cc1 ; c1ccc(-c2nc(-c3ccc4ccc5ccc6nc(-c7ccccc7)oc6c5c4c3)nc(-c3cccc4oc5ccccc5c34)n2)cc1]

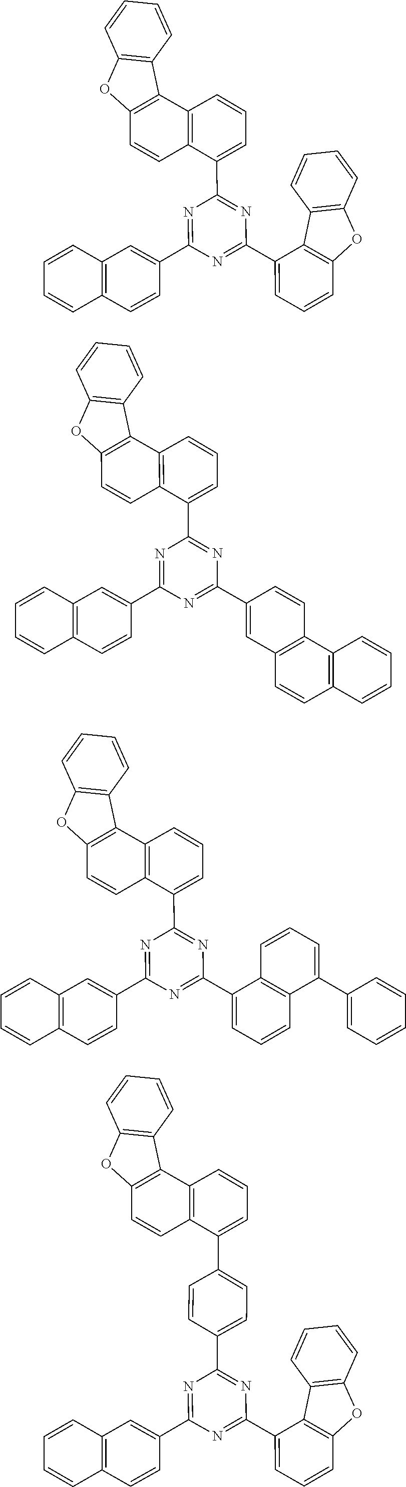

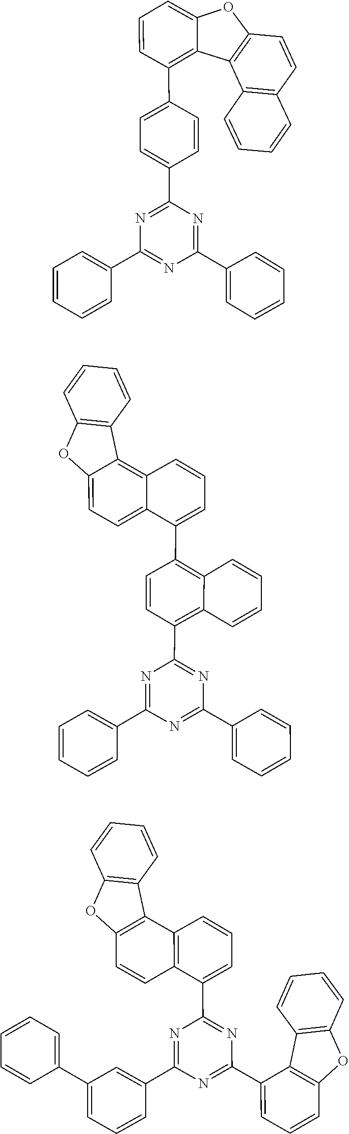 [SMILES: c1ccc(-c2nc(-c3ccccc3)nc(-c3ccc(-c4cccc5oc6ccc7ccccc7c6c45)cc3)n2)cc1 ; c1ccc(-c2nc(-c3ccccc3)nc(-c3ccc(-c4cccc5c4ccc4oc6ccccc6c45)c4ccccc34)n2)cc1 ; c1ccc(-c2cccc(-c3nc(-c4cccc5c4ccc4oc6ccccc6c45)nc(-c4cccc5oc6ccccc6c45)n3)c2)cc1]

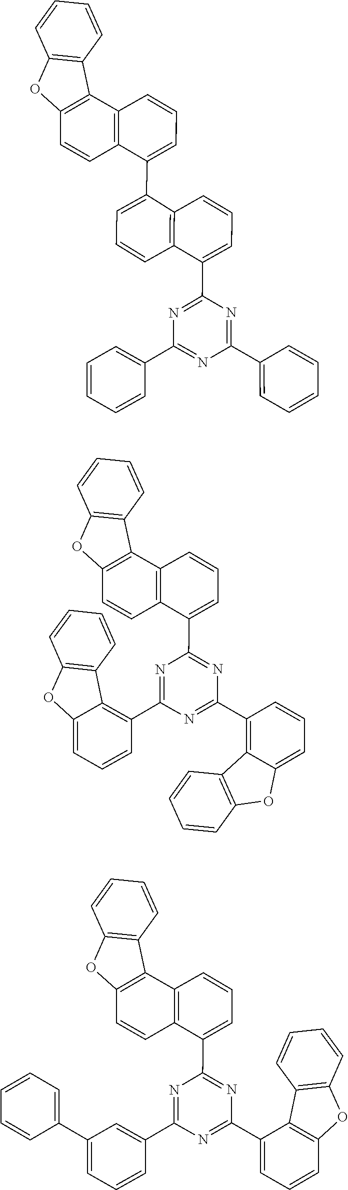 [SMILES: c1ccc(-c2nc(-c3ccccc3)nc(-c3cccc4c(-c5cccc6c5ccc5oc7ccccc7c56)cccc34)n2)cc1 ; c1ccc2c(c1)oc1cccc(-c3nc(-c4cccc5c4ccc4oc6ccccc6c45)nc(-c4cccc5oc6ccccc6c45)n3)c12 ; c1ccc(-c2cccc(-c3nc(-c4cccc5c4ccc4oc6ccccc6c45)nc(-c4cccc5oc6ccccc6c45)n3)c2)cc1]

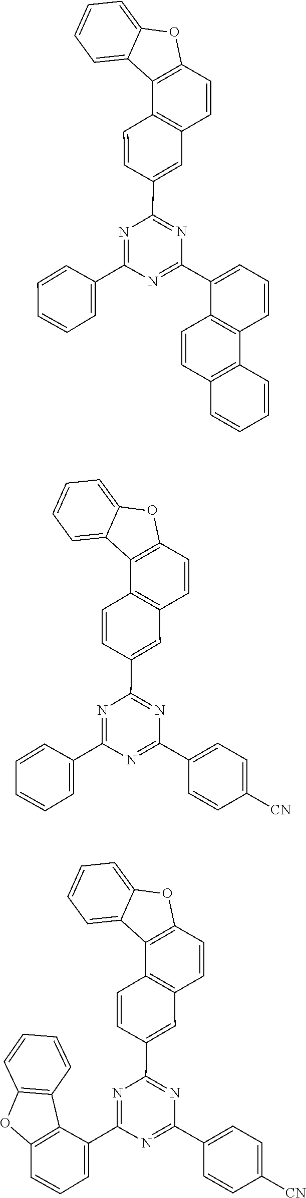 [SMILES: c1ccc(-c2nc(-c3ccc4c(ccc5oc6ccccc6c54)c3)nc(-c3cccc4c3ccc3ccccc34)n2)cc1 ; N#Cc1ccc(-c2nc(-c3ccccc3)nc(-c3ccc4c(ccc5oc6ccccc6c54)c3)n2)cc1 ; N#Cc1ccc(-c2nc(-c3ccc4c(ccc5oc6ccccc6c54)c3)nc(-c3cccc4oc5ccccc5c34)n2)cc1]

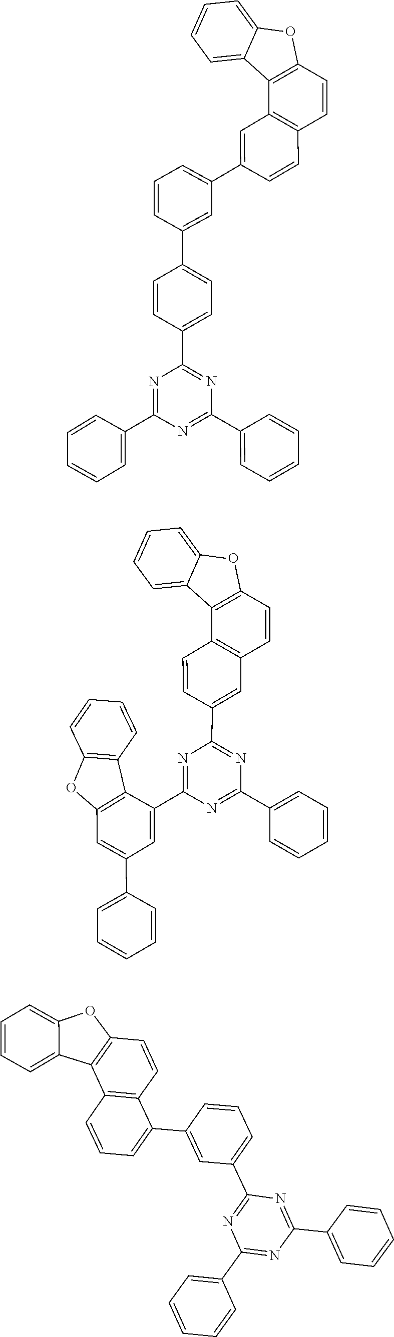 [SMILES: c1ccc(-c2nc(-c3ccccc3)nc(-c3ccc(-c4cccc(-c5ccc6ccc7oc8ccccc8c7c6c5)c4)cc3)n2)cc1 ; c1ccc(-c2cc(-c3nc(-c4ccccc4)nc(-c4ccc5c(ccc6oc7ccccc7c65)c4)n3)c3c(c2)oc2ccccc23)cc1 ; c1ccc(-c2nc(-c3ccccc3)nc(-c3cccc(-c4cccc5c4ccc4oc6ccccc6c45)c3)n2)cc1]

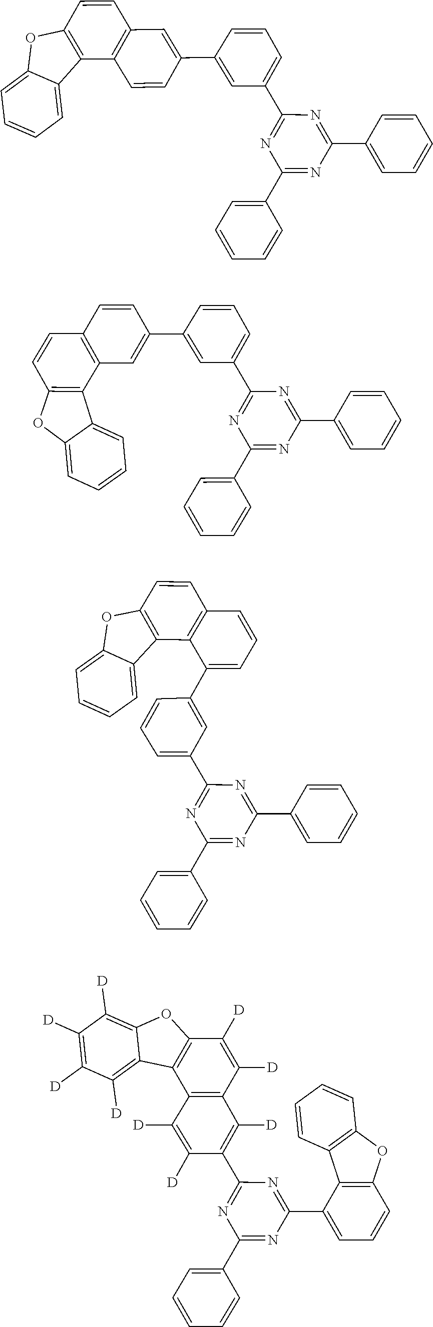

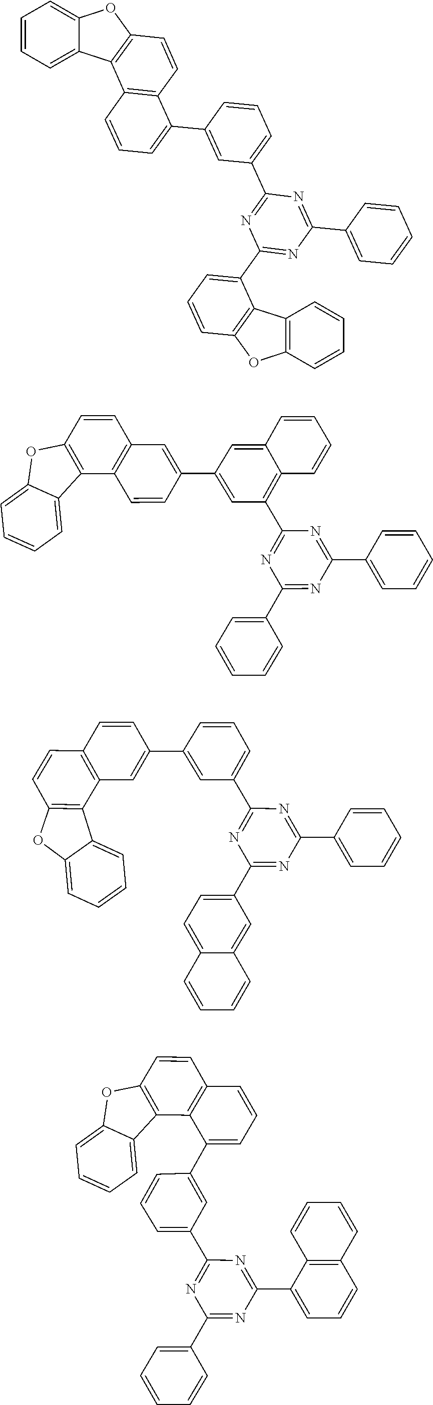 [SMILES: c1ccc(-c2nc(-c3cccc(-c4cccc5c4ccc4oc6ccccc6c45)c3)nc(-c3cccc4oc5ccccc5c34)n2)cc1 ; c1ccc(-c2nc(-c3ccccc3)nc(-c3cc(-c4ccc5c(ccc6oc7ccccc7c65)c4)cc4ccccc34)n2)cc1 ; c1ccc(-c2nc(-c3cccc(-c4ccc5ccc6oc7ccccc7c6c5c4)c3)nc(-c3ccc4ccccc4c3)n2)cc1 ; c1ccc(-c2nc(-c3cccc(-c4cccc5ccc6oc7ccccc7c6c45)c3)nc(-c3cccc4ccccc34)n2)cc1]

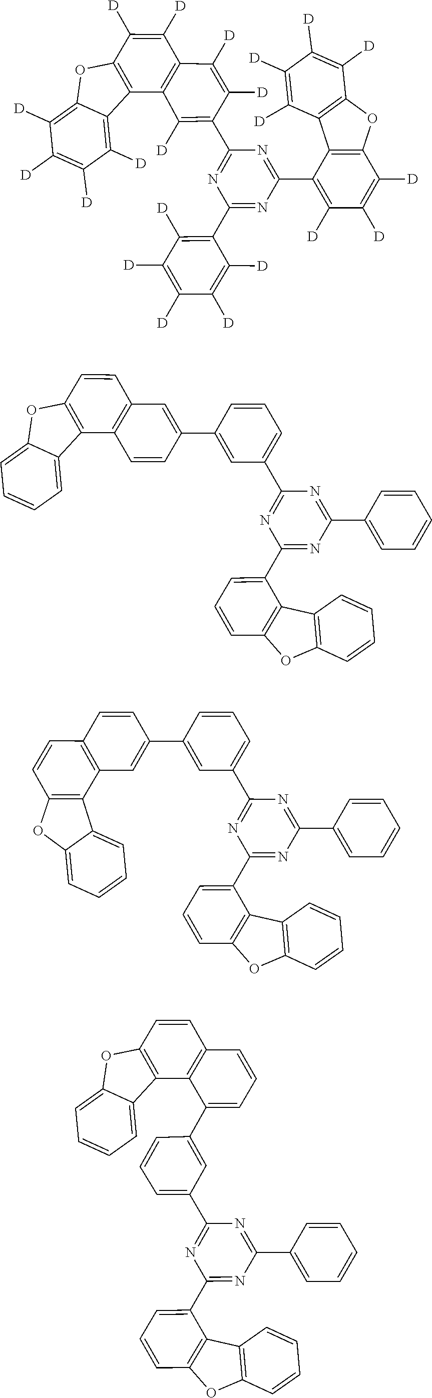 [SMILES: [2H]c1c([2H])c([2H])c(-c2nc(-c3c([2H])c([2H])c4c([2H])c([2H])c5oc6c([2H])c([2H])c([2H])c([2H])c6c5c4c3[2H])nc(-c3c([2H])c([2H])c([2H])c4oc5c([2H])c([2H])c([2H])c([2H])c5c34)n2)c([2H])c1[2H] ; c1ccc(-c2nc(-c3cccc(-c4ccc5c(ccc6oc7ccccc7c65)c4)c3)nc(-c3cccc4oc5ccccc5c34)n2)cc1 ; c1ccc(-c2nc(-c3cccc(-c4ccc5ccc6oc7ccccc7c6c5c4)c3)nc(-c3cccc4oc5ccccc5c34)n2)cc1 ; c1ccc(-c2nc(-c3cccc(-c4cccc5ccc6oc7ccccc7c6c45)c3)nc(-c3cccc4oc5ccccc5c34)n2)cc1]

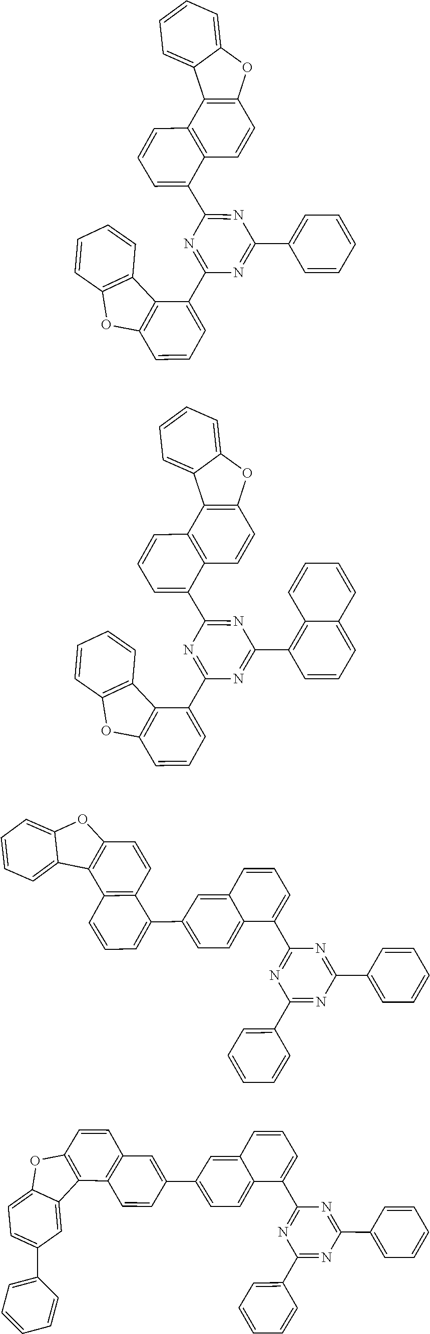 [SMILES: c1ccc(-c2nc(-c3cccc4c3ccc3oc5ccccc5c34)nc(-c3cccc4oc5ccccc5c34)n2)cc1 ; c1ccc2c(-c3nc(-c4cccc5c4ccc4oc6ccccc6c45)nc(-c4cccc5oc6ccccc6c45)n3)cccc2c1 ; c1ccc(-c2nc(-c3ccccc3)nc(-c3cccc4cc(-c5cccc6c5ccc5oc7ccccc7c56)ccc34)n2)cc1 ; c1ccc(-c2ccc3oc4ccc5cc(-c6ccc7c(-c8nc(-c9ccccc9)nc(-c9ccccc9)n8)cccc7c6)ccc5c4c3c2)cc1]

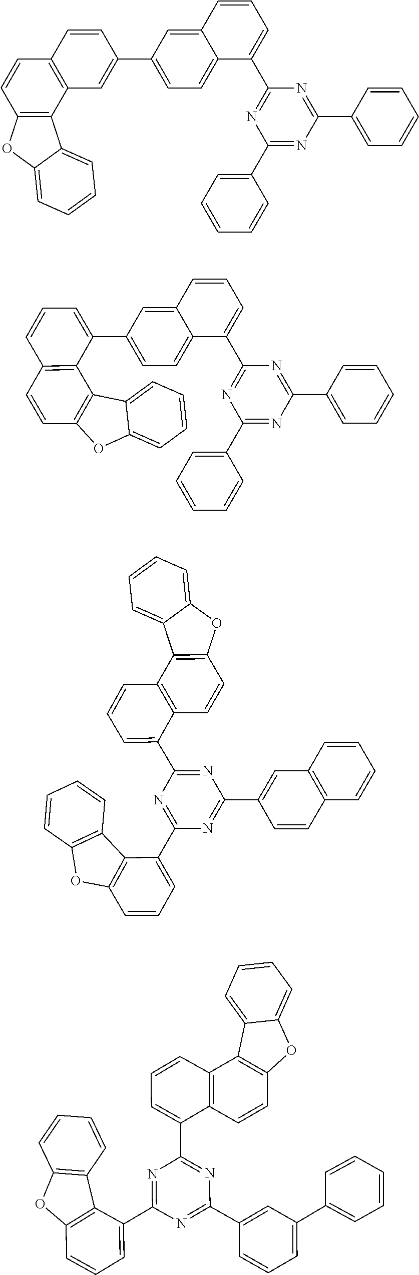 [SMILES: c1ccc(-c2nc(-c3ccccc3)nc(-c3cccc4cc(-c5ccc6ccc7oc8ccccc8c7c6c5)ccc34)n2)cc1 ; c1ccc(-c2nc(-c3ccccc3)nc(-c3cccc4cc(-c5cccc6ccc7oc8ccccc8c7c56)ccc34)n2)cc1 ; c1ccc2cc(-c3nc(-c4cccc5c4ccc4oc6ccccc6c45)nc(-c4cccc5oc6ccccc6c45)n3)ccc2c1 ; c1ccc(-c2cccc(-c3nc(-c4cccc5c4ccc4oc6ccccc6c45)nc(-c4cccc5oc6ccccc6c45)n3)c2)cc1]

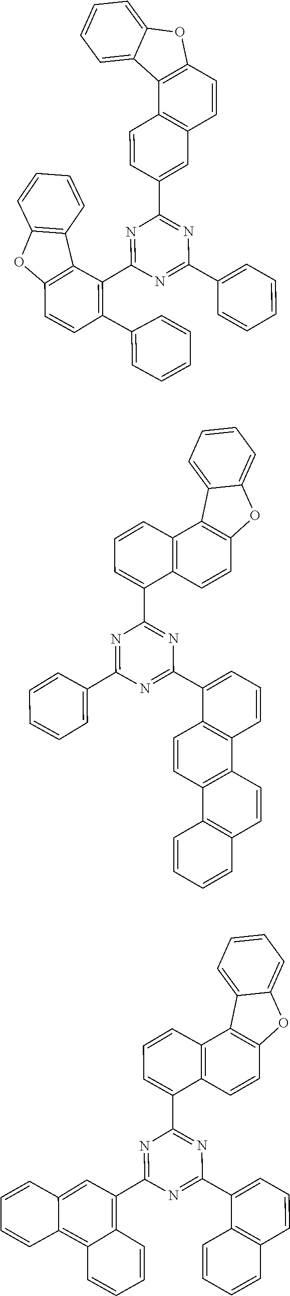 [SMILES: c1ccc(-c2nc(-c3ccc4c(ccc5oc6ccccc6c54)c3)nc(-c3c(-c4ccccc4)ccc4oc5ccccc5c34)n2)cc1 ; c1ccc(-c2nc(-c3cccc4c3ccc3c5ccccc5ccc43)nc(-c3cccc4c3ccc3oc5ccccc5c34)n2)cc1 ; c1ccc2c(-c3nc(-c4cc5ccccc5c5ccccc45)nc(-c4cccc5c4ccc4oc6ccccc6c45)n3)cccc2c1]

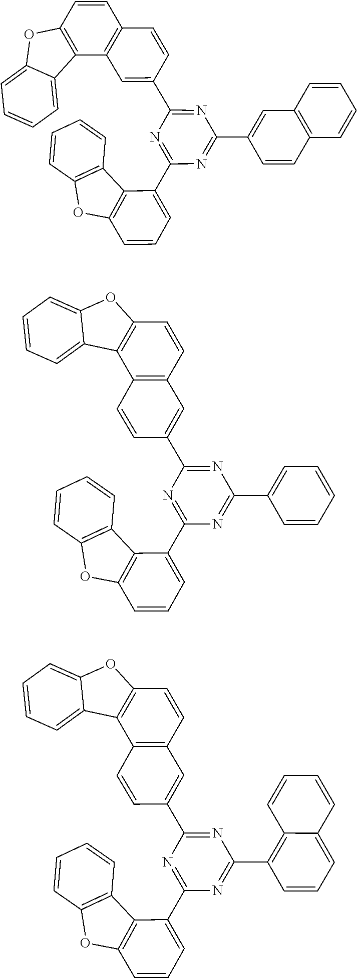 [SMILES: c1ccc2cc(-c3nc(-c4ccc5ccc6oc7ccccc7c6c5c4)nc(-c4cccc5oc6ccccc6c45)n3)ccc2c1 ; c1ccc(-c2nc(-c3ccc4c(ccc5oc6ccccc6c54)c3)nc(-c3cccc4oc5ccccc5c34)n2)cc1 ; c1ccc2c(-c3nc(-c4ccc5c(ccc6oc7ccccc7c65)c4)nc(-c4cccc5oc6ccccc6c45)n3)cccc2c1]

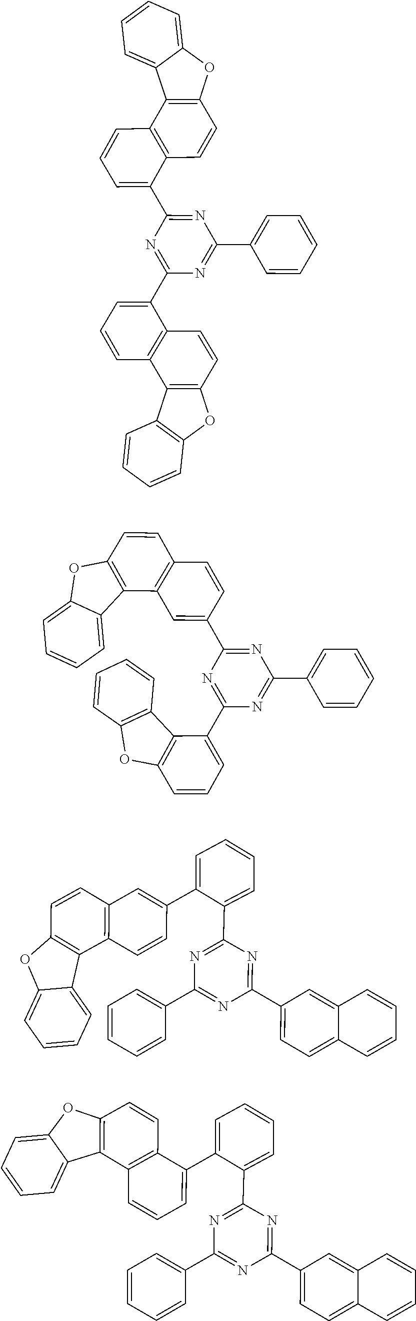 [SMILES: c1ccc(-c2nc(-c3cccc4c3ccc3oc5ccccc5c34)nc(-c3cccc4c3ccc3oc5ccccc5c34)n2)cc1 ; c1ccc(-c2nc(-c3ccc4ccc5oc6ccccc6c5c4c3)nc(-c3cccc4oc5ccccc5c34)n2)cc1 ; c1ccc(-c2nc(-c3ccc4ccccc4c3)nc(-c3ccccc3-c3ccc4c(ccc5oc6ccccc6c54)c3)n2)cc1 ; c1ccc(-c2nc(-c3ccc4ccccc4c3)nc(-c3ccccc3-c3cccc4c3ccc3oc5ccccc5c34)n2)cc1]

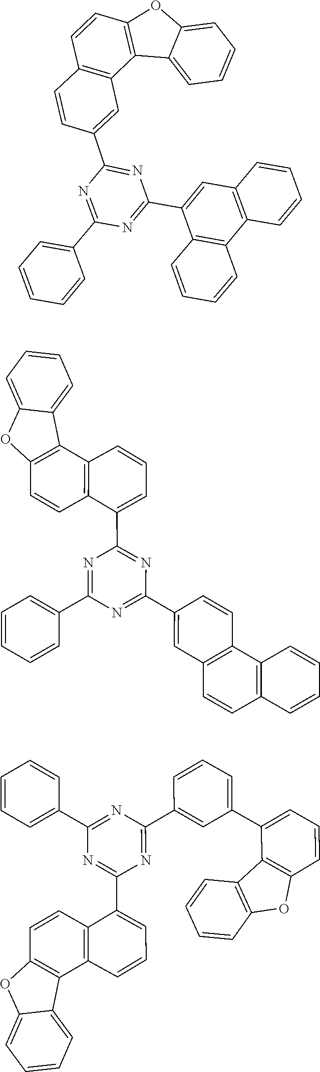 [SMILES: c1ccc(-c2nc(-c3ccc4ccc5oc6ccccc6c5c4c3)nc(-c3cc4ccccc4c4ccccc34)n2)cc1 ; c1ccc(-c2nc(-c3ccc4c(ccc5ccccc54)c3)nc(-c3cccc4c3ccc3oc5ccccc5c34)n2)cc1 ; c1ccc(-c2nc(-c3cccc(-c4cccc5oc6ccccc6c45)c3)nc(-c3cccc4c3ccc3oc5ccccc5c34)n2)cc1]

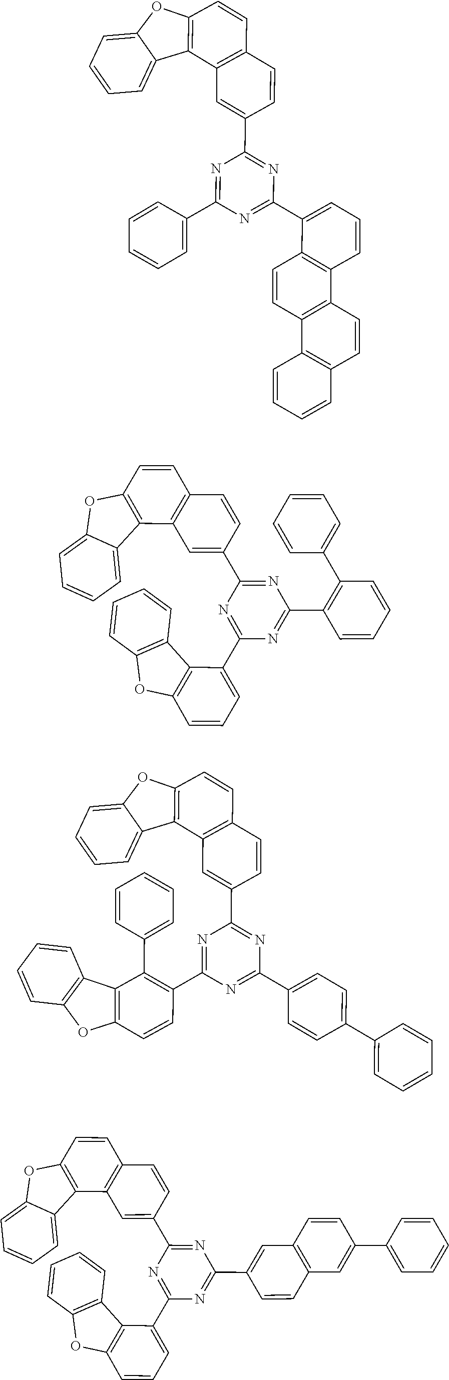 [SMILES: c1ccc(-c2nc(-c3ccc4ccc5oc6ccccc6c5c4c3)nc(-c3cccc4c3ccc3c5ccccc5ccc43)n2)cc1 ; c1ccc(-c2ccccc2-c2nc(-c3ccc4ccc5oc6ccccc6c5c4c3)nc(-c3cccc4oc5ccccc5c34)n2)cc1 ; c1ccc(-c2ccc(-c3nc(-c4ccc5ccc6oc7ccccc7c6c5c4)nc(-c4ccc5oc6ccccc6c5c4-c4ccccc4)n3)cc2)cc1 ; c1ccc(-c2ccc3cc(-c4nc(-c5ccc6ccc7oc8ccccc8c7c6c5)nc(-c5cccc6oc7ccccc7c56)n4)ccc3c2)cc1]

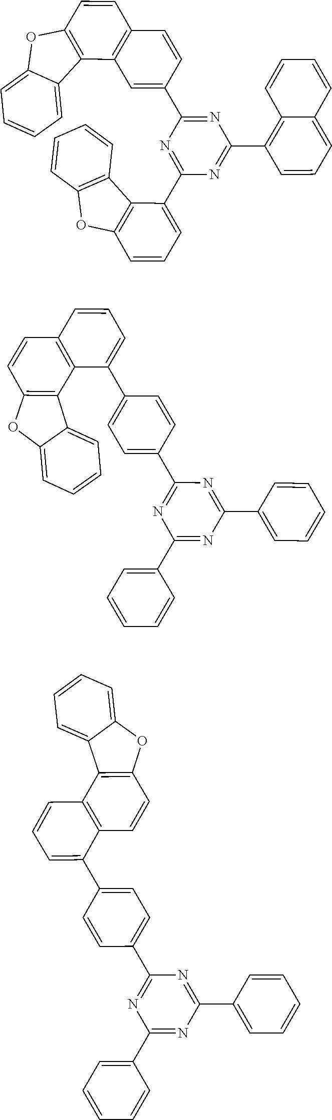 [SMILES: c1ccc2c(-c3nc(-c4ccc5ccc6oc7ccccc7c6c5c4)nc(-c4cccc5oc6ccccc6c45)n3)cccc2c1 ; c1ccc(-c2nc(-c3ccccc3)nc(-c3ccc(-c4cccc5ccc6oc7ccccc7c6c45)cc3)n2)cc1 ; c1ccc(-c2nc(-c3ccccc3)nc(-c3ccc(-c4cccc5c4ccc4oc6ccccc6c45)cc3)n2)cc1]

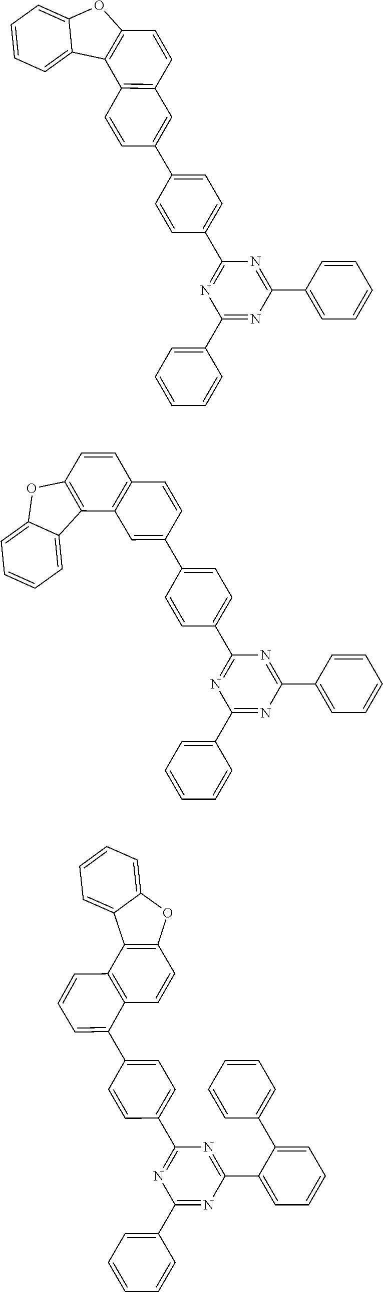 [SMILES: c1ccc(-c2nc(-c3ccccc3)nc(-c3ccc(-c4ccc5c(ccc6oc7ccccc7c65)c4)cc3)n2)cc1 ; c1ccc(-c2nc(-c3ccccc3)nc(-c3ccc(-c4ccc5ccc6oc7ccccc7c6c5c4)cc3)n2)cc1 ; c1ccc(-c2nc(-c3ccc(-c4cccc5c4ccc4oc6ccccc6c45)cc3)nc(-c3ccccc3-c3ccccc3)n2)cc1]

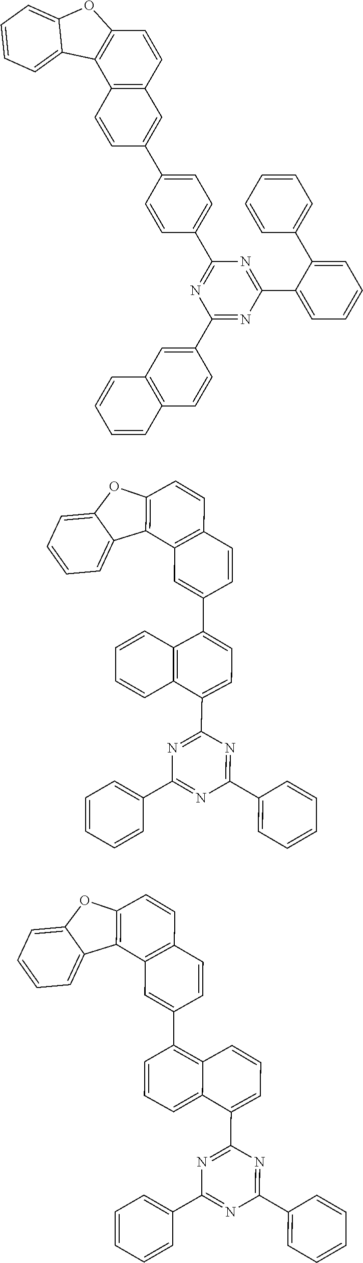 [SMILES: c1ccc(-c2ccccc2-c2nc(-c3ccc(-c4ccc5c(ccc6oc7ccccc7c65)c4)cc3)nc(-c3ccc4ccccc4c3)n2)cc1 ; c1ccc(-c2nc(-c3ccccc3)nc(-c3ccc(-c4ccc5ccc6oc7ccccc7c6c5c4)c4ccccc34)n2)cc1 ; c1ccc(-c2nc(-c3ccccc3)nc(-c3cccc4c(-c5ccc6ccc7oc8ccccc8c7c6c5)cccc34)n2)cc1]

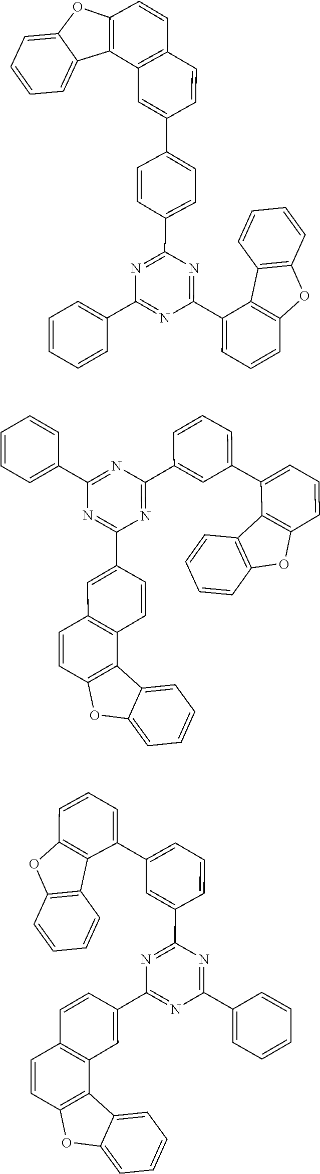 [SMILES: c1ccc(-c2nc(-c3ccc(-c4ccc5ccc6oc7ccccc7c6c5c4)cc3)nc(-c3cccc4oc5ccccc5c34)n2)cc1 ; c1ccc(-c2nc(-c3cccc(-c4cccc5oc6ccccc6c45)c3)nc(-c3ccc4c(ccc5oc6ccccc6c54)c3)n2)cc1 ; c1ccc(-c2nc(-c3cccc(-c4cccc5oc6ccccc6c45)c3)nc(-c3ccc4ccc5oc6ccccc6c5c4c3)n2)cc1]

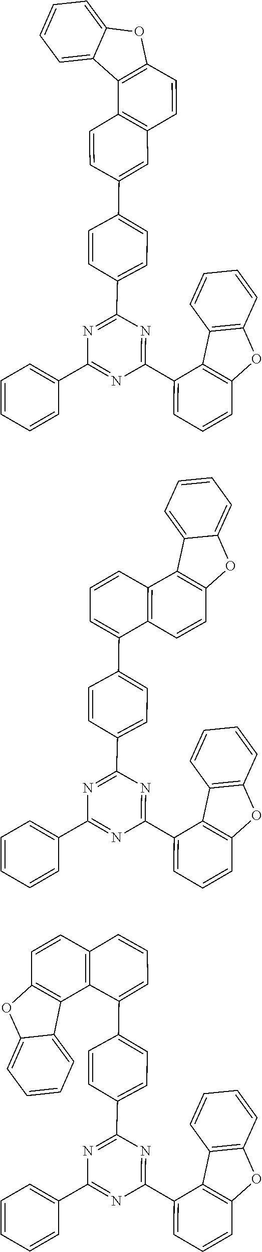 [SMILES: c1ccc(-c2nc(-c3ccc(-c4ccc5c(ccc6oc7ccccc7c65)c4)cc3)nc(-c3cccc4oc5ccccc5c34)n2)cc1 ; c1ccc(-c2nc(-c3ccc(-c4cccc5c4ccc4oc6ccccc6c45)cc3)nc(-c3cccc4oc5ccccc5c34)n2)cc1 ; c1ccc(-c2nc(-c3ccc(-c4cccc5ccc6oc7ccccc7c6c45)cc3)nc(-c3cccc4oc5ccccc5c34)n2)cc1]

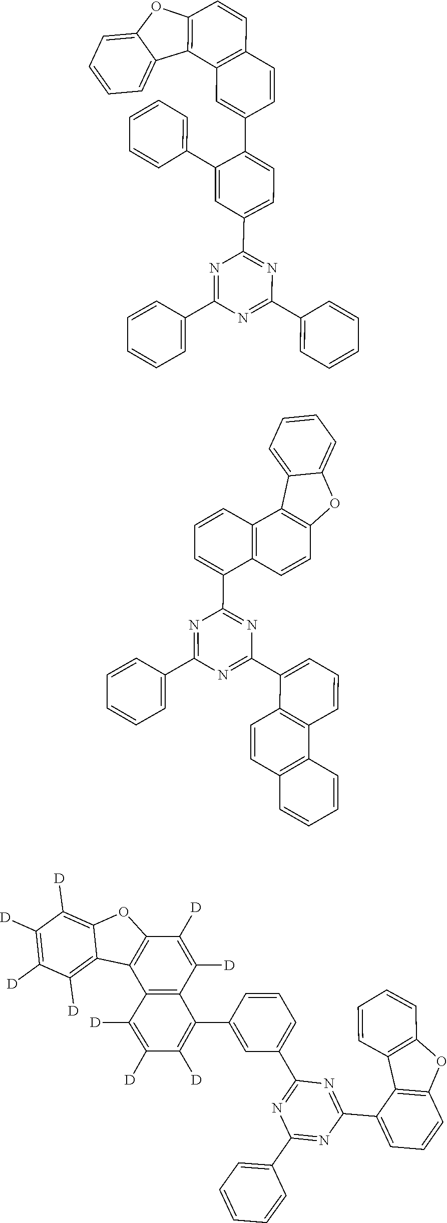 [SMILES: c1ccc(-c2nc(-c3ccccc3)nc(-c3ccc(-c4ccc5ccc6oc7ccccc7c6c5c4)c(-c4ccccc4)c3)n2)cc1 ; c1ccc(-c2nc(-c3cccc4c3ccc3ccccc34)nc(-c3cccc4c3ccc3oc5ccccc5c34)n2)cc1 ; [2H]c1c([2H])c([2H])c2c(oc3c([2H])c([2H])c4c(-c5cccc(-c6nc(-c7ccccc7)nc(-c7cccc8oc9ccccc9c78)n6)c5)c([2H])c([2H])c([2H])c4c32)c1[2H]]

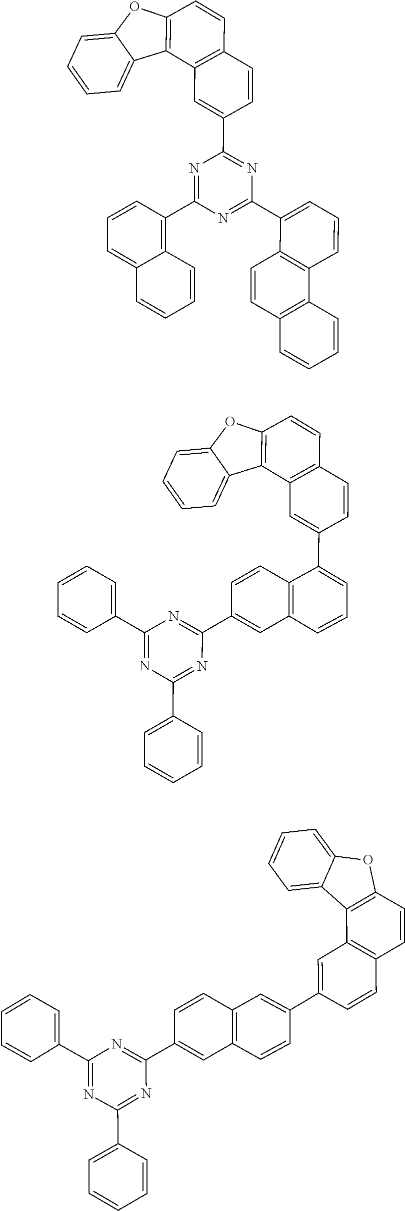 [SMILES: c1ccc2c(-c3nc(-c4ccc5ccc6oc7ccccc7c6c5c4)nc(-c4cccc5c4ccc4ccccc45)n3)cccc2c1 ; c1ccc(-c2nc(-c3ccccc3)nc(-c3ccc4c(-c5ccc6ccc7oc8ccccc8c7c6c5)cccc4c3)n2)cc1 ; c1ccc(-c2nc(-c3ccccc3)nc(-c3ccc4cc(-c5ccc6ccc7oc8ccccc8c7c6c5)ccc4c3)n2)cc1]

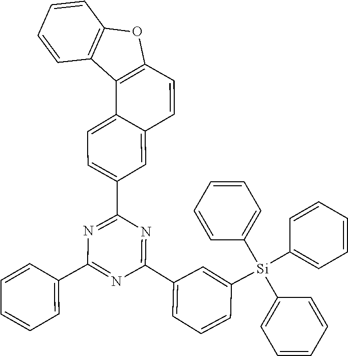 [SMILES: c1ccc(-c2nc(-c3cccc([Si](c4ccccc4)(c4ccccc4)c4ccccc4)c3)nc(-c3ccc4c(ccc5oc6ccccc6c54)c3)n2)cc1]

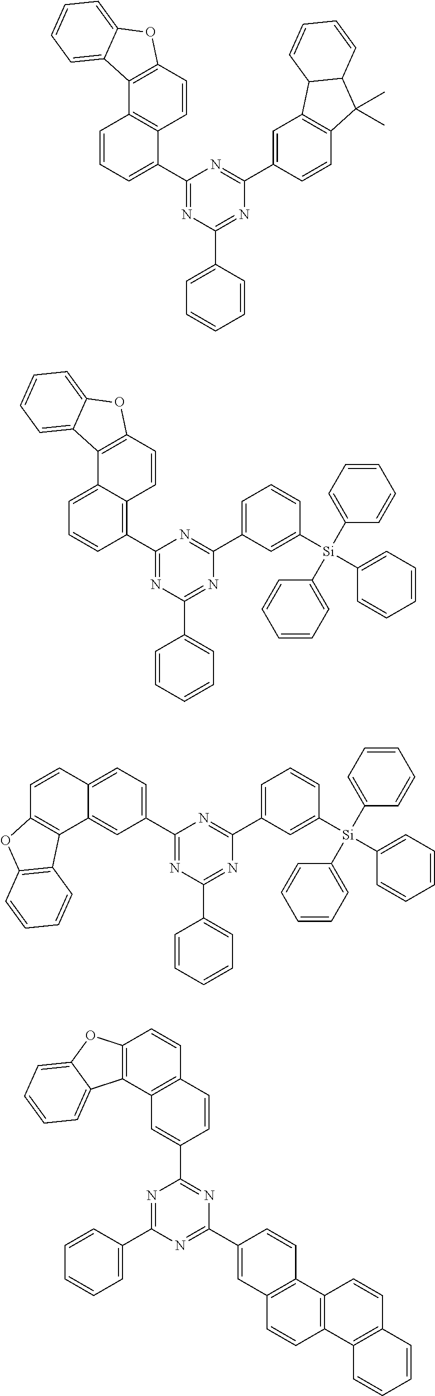 [SMILES: CC1(C)c2ccc(-c3nc(-c4ccccc4)nc(-c4cccc5c4ccc4oc6ccccc6c45)n3)cc2C2C=CC=CC21 ; c1ccc(-c2nc(-c3cccc([Si](c4ccccc4)(c4ccccc4)c4ccccc4)c3)nc(-c3cccc4c3ccc3oc5ccccc5c34)n2)cc1 ; c1ccc(-c2nc(-c3cccc([Si](c4ccccc4)(c4ccccc4)c4ccccc4)c3)nc(-c3ccc4ccc5oc6ccccc6c5c4c3)n2)cc1 ; c1ccc(-c2nc(-c3ccc4c(ccc5c6ccccc6ccc45)c3)nc(-c3ccc4ccc5oc6ccccc6c5c4c3)n2)cc1]

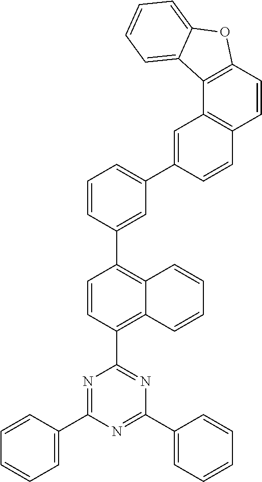 [SMILES: c1ccc(-c2nc(-c3ccccc3)nc(-c3ccc(-c4cccc(-c5ccc6ccc7oc8ccccc8c7c6c5)c4)c4ccccc34)n2)cc1]

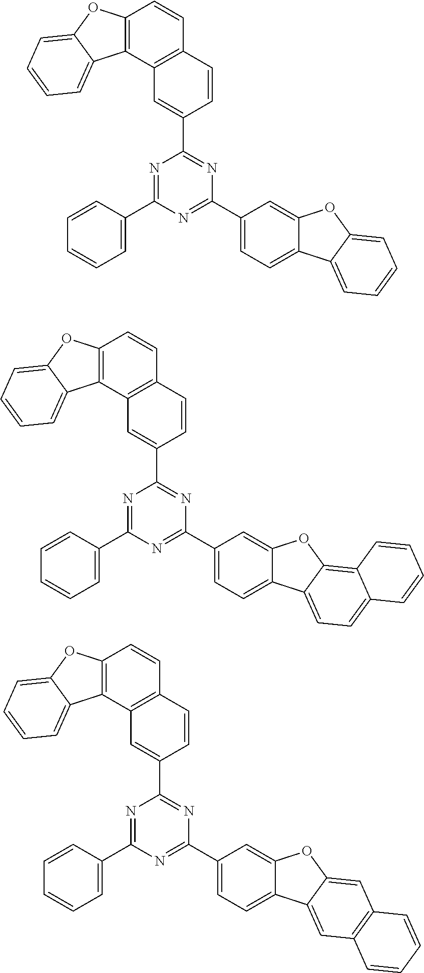 [SMILES: c1ccc(-c2nc(-c3ccc4c(c3)oc3ccccc34)nc(-c3ccc4ccc5oc6ccccc6c5c4c3)n2)cc1 ; c1ccc(-c2nc(-c3ccc4c(c3)oc3c5ccccc5ccc43)nc(-c3ccc4ccc5oc6ccccc6c5c4c3)n2)cc1 ; c1ccc(-c2nc(-c3ccc4c(c3)oc3cc5ccccc5cc34)nc(-c3ccc4ccc5oc6ccccc6c5c4c3)n2)cc1]

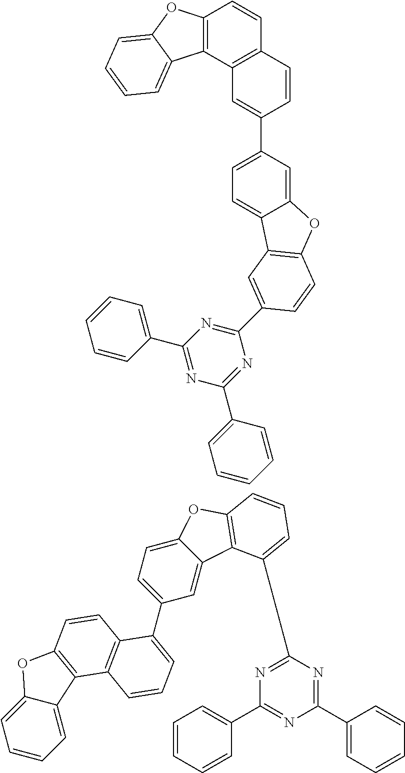

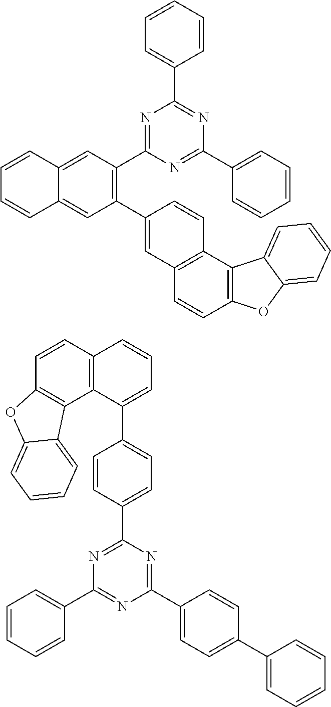 [SMILES: c1ccc(-c2nc(-c3ccccc3)nc(-c3cc4ccccc4cc3-c3ccc4c(ccc5oc6ccccc6c54)c3)n2)cc1 ; c1ccc(-c2ccc(-c3nc(-c4ccccc4)nc(-c4ccc(-c5cccc6ccc7oc8ccccc8c7c56)cc4)n3)cc2)cc1]

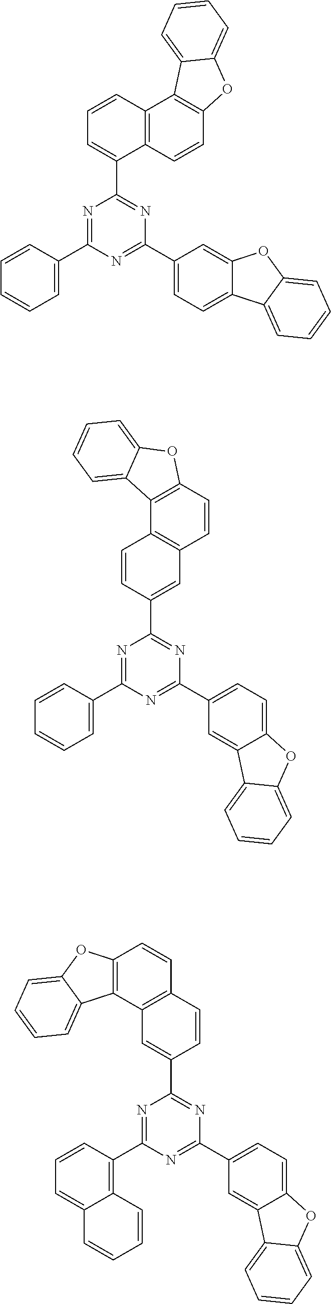 [SMILES: c1ccc(-c2nc(-c3ccc4c(c3)oc3ccccc34)nc(-c3cccc4c3ccc3oc5ccccc5c34)n2)cc1 ; c1ccc(-c2nc(-c3ccc4c(ccc5oc6ccccc6c54)c3)nc(-c3ccc4oc5ccccc5c4c3)n2)cc1 ; c1ccc2c(-c3nc(-c4ccc5oc6ccccc6c5c4)nc(-c4ccc5ccc6oc7ccccc7c6c5c4)n3)cccc2c1]

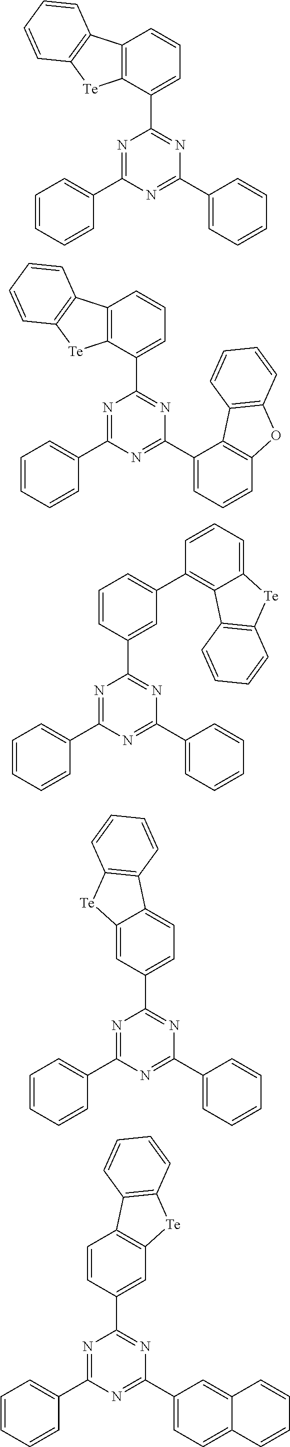

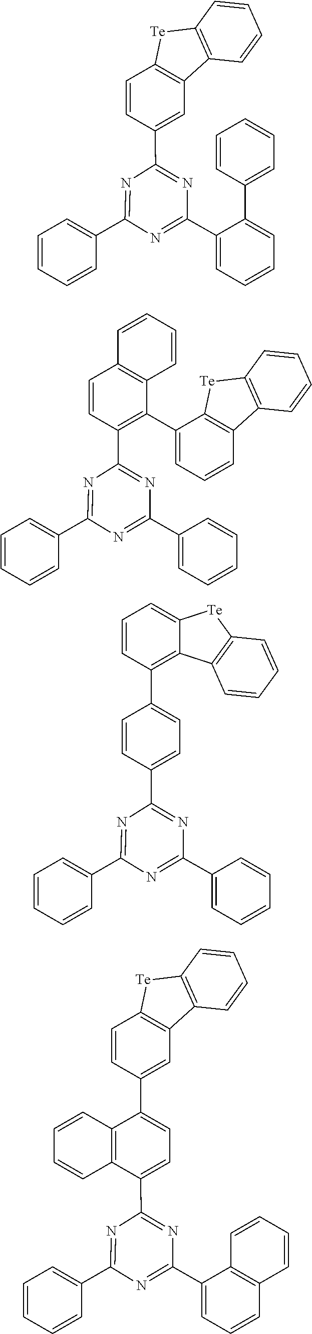

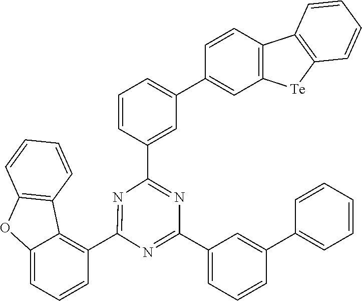

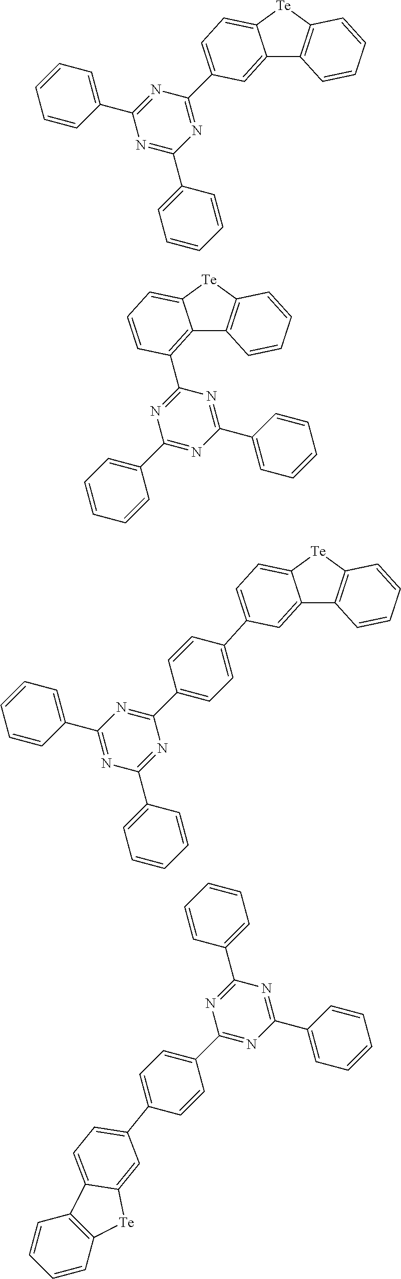

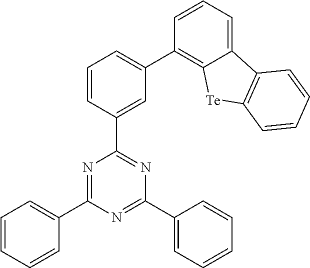

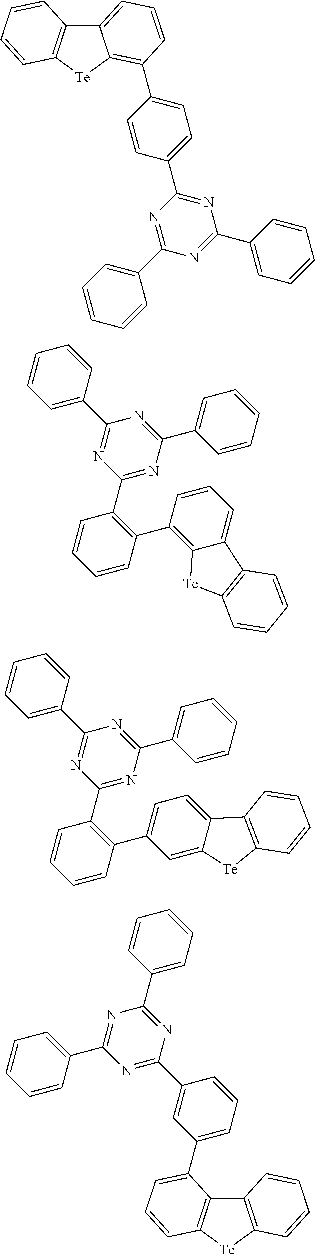

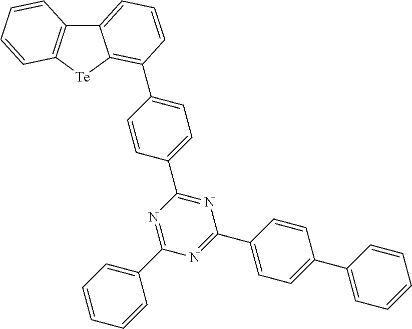

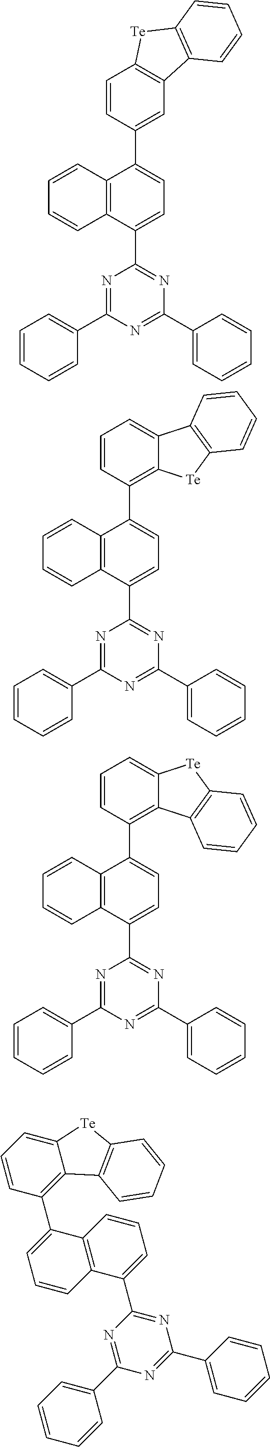

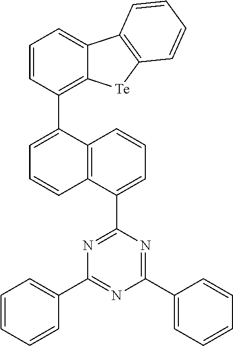

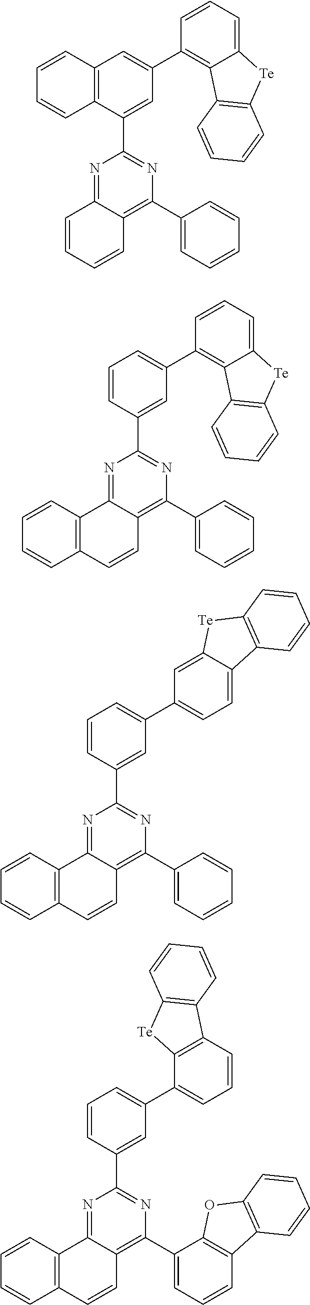

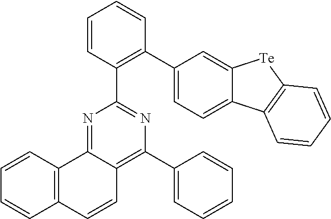

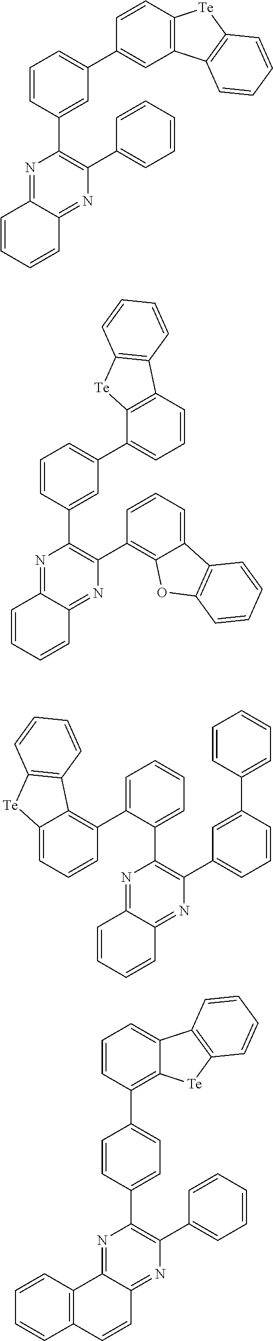

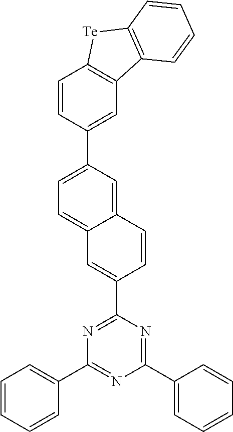

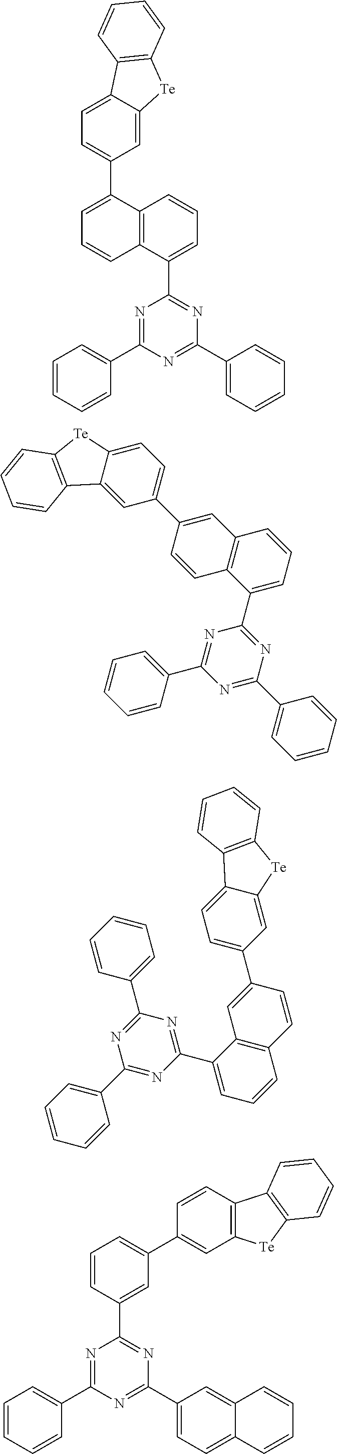

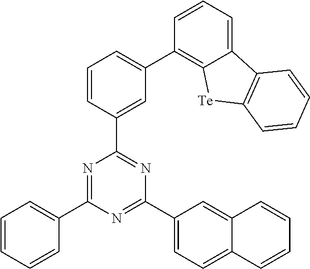

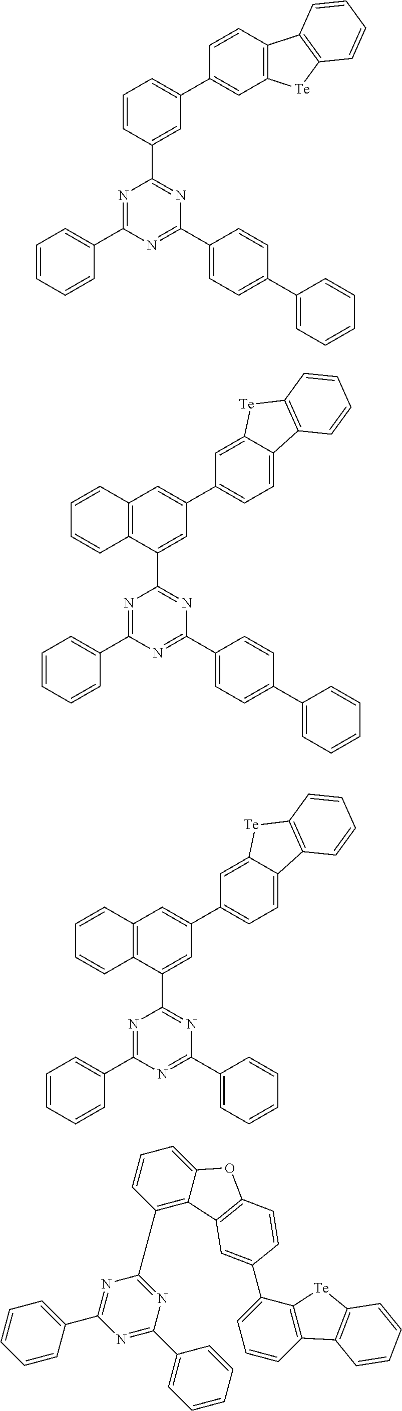

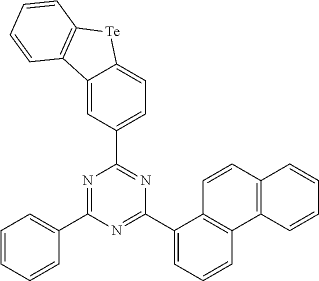

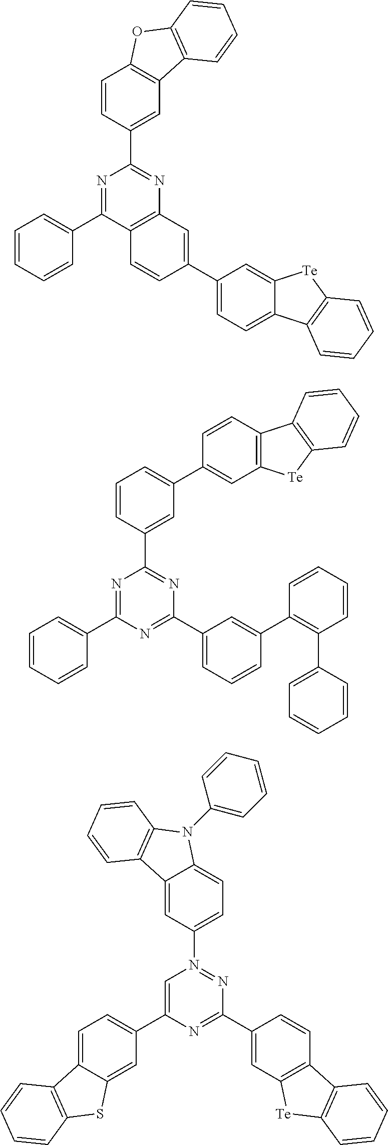

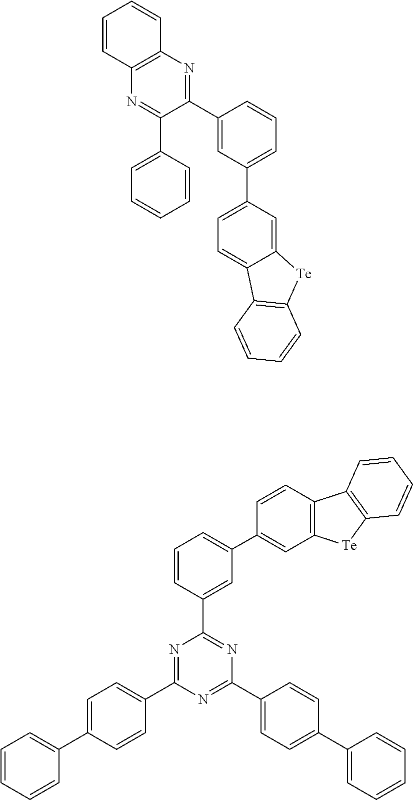

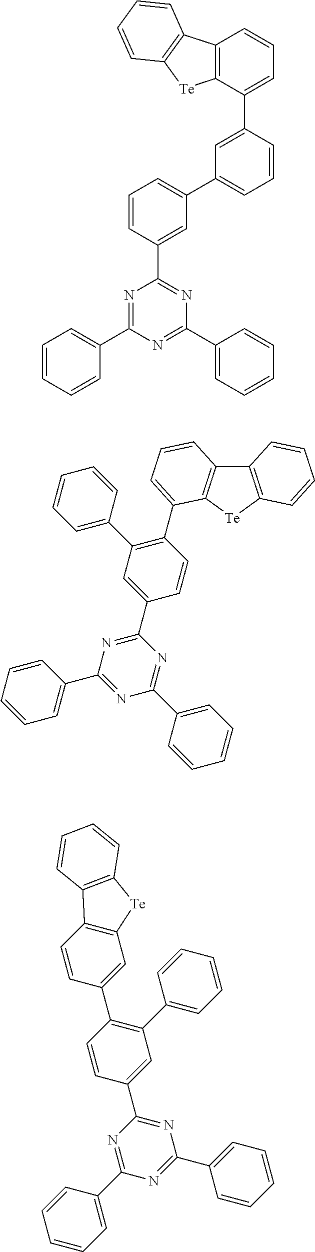

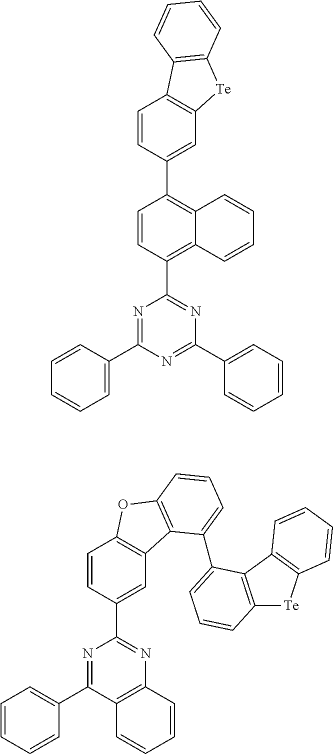

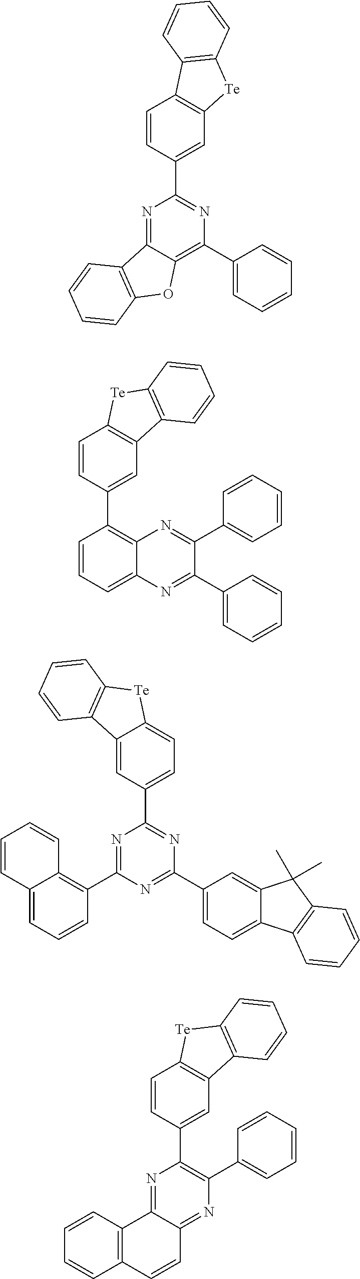

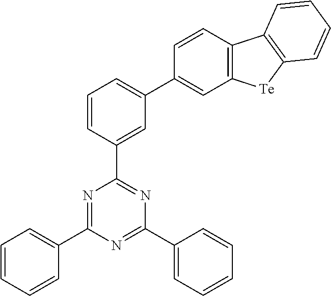

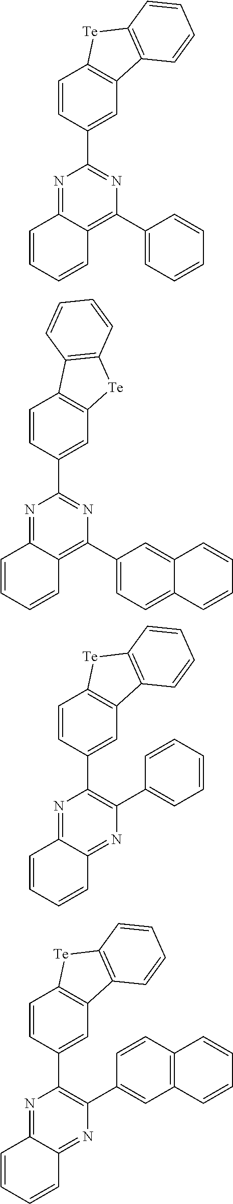

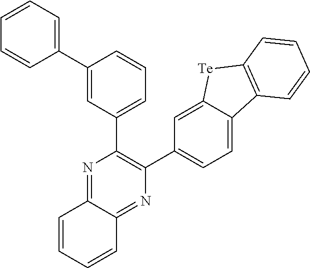

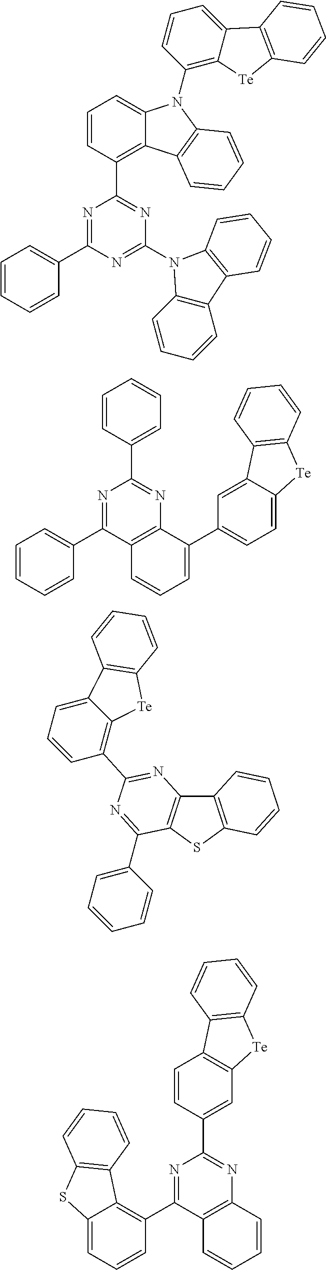

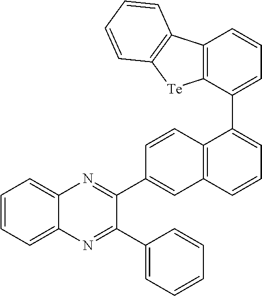

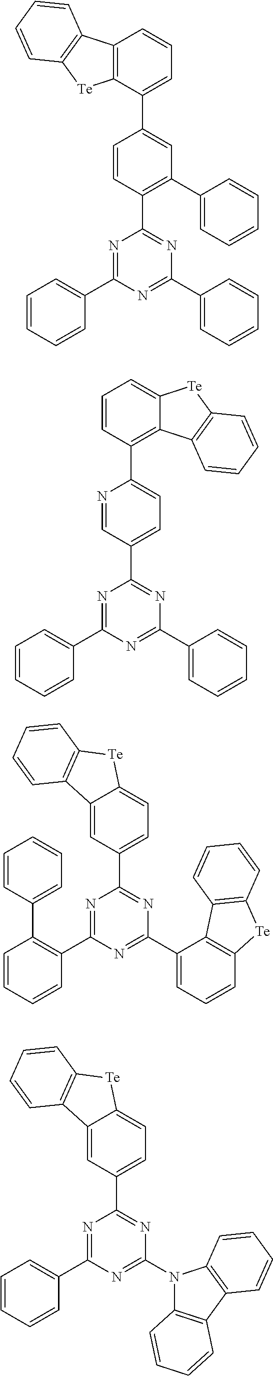

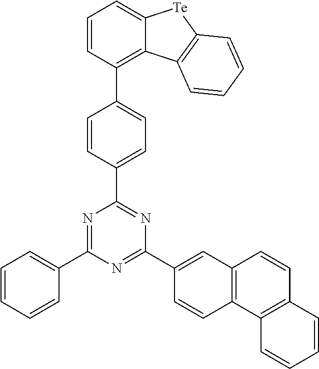

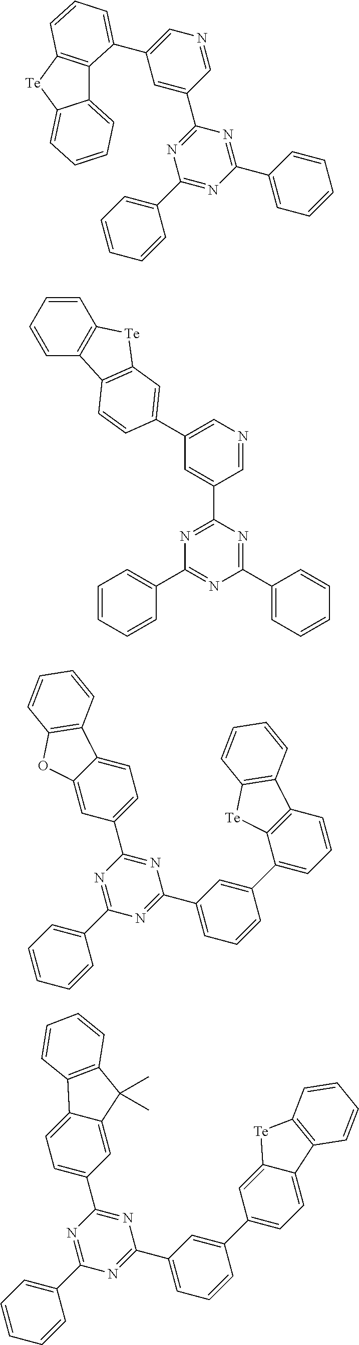

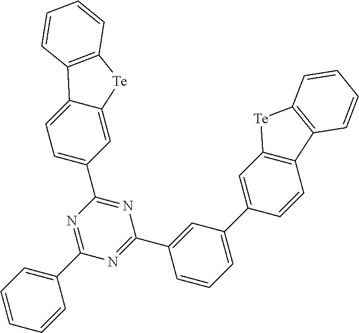

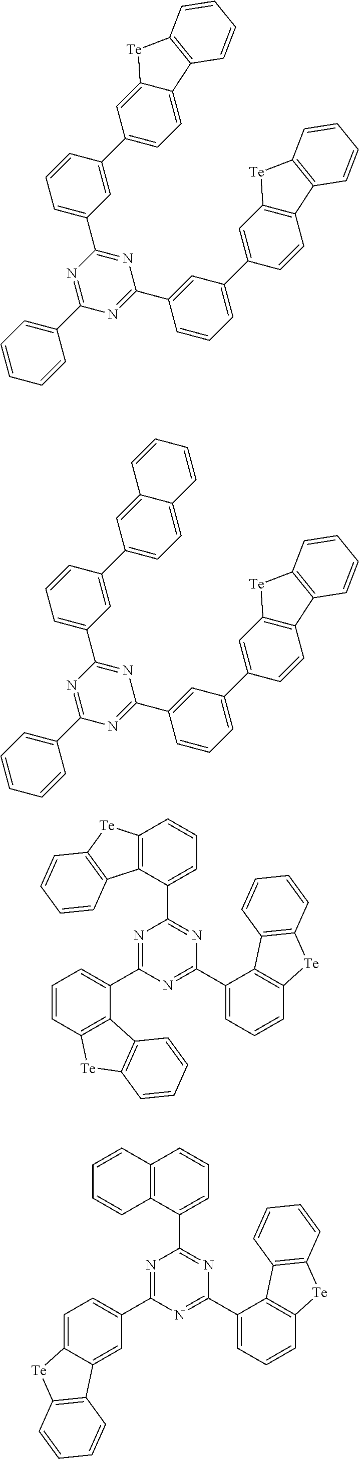

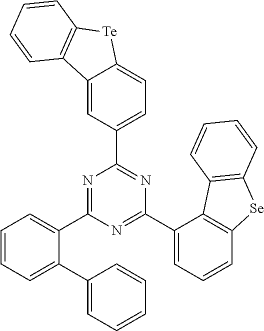

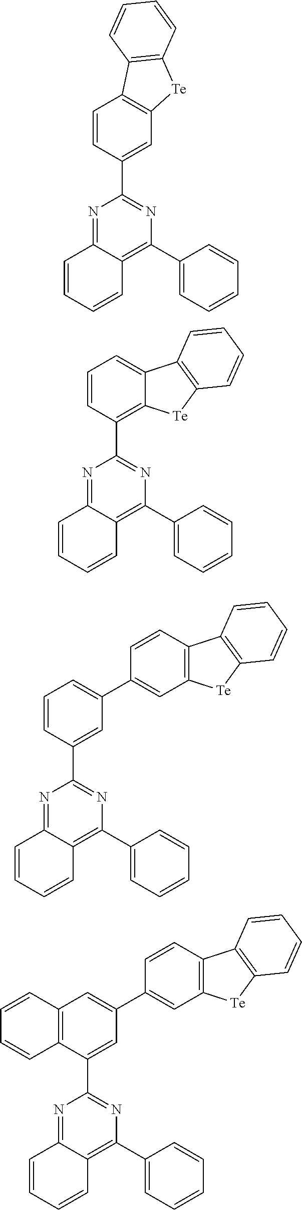

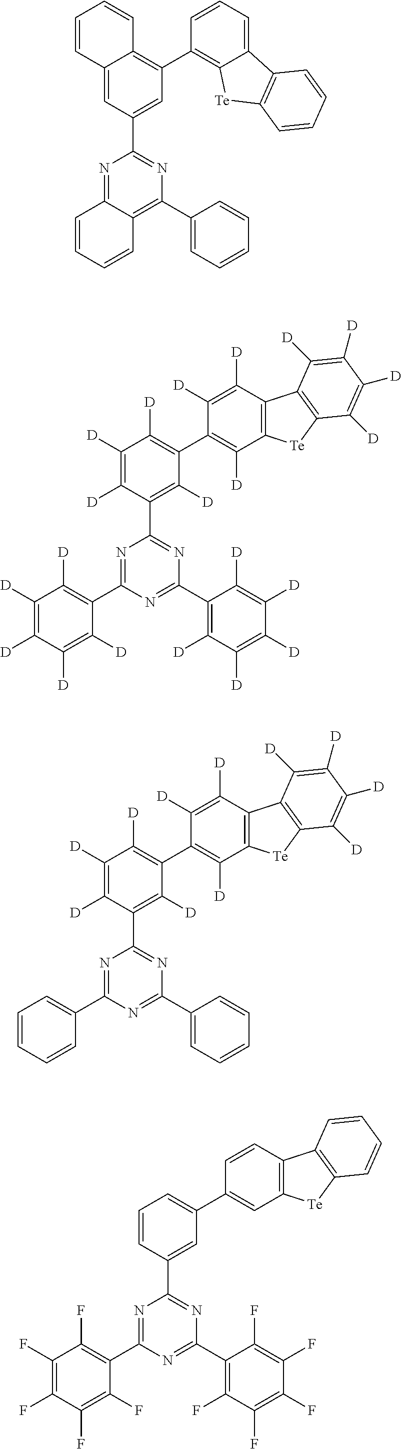

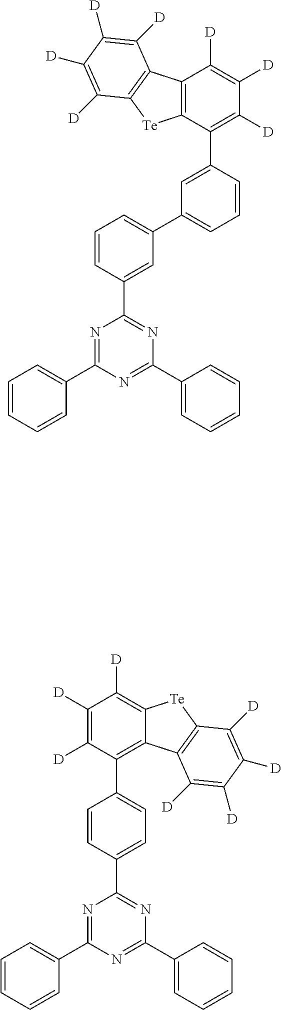

The compound represented by formula 15 may be at least one selected from the group consisting of the following compounds, but is not limited thereto.

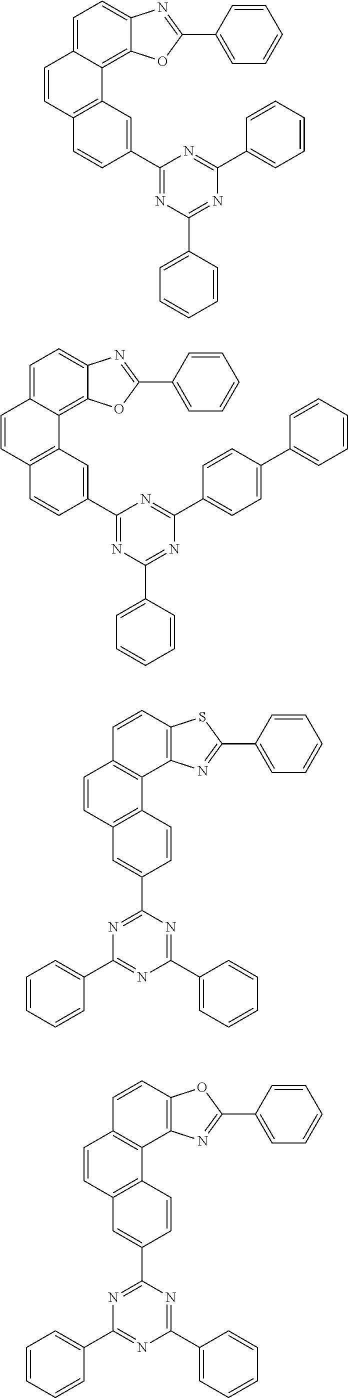

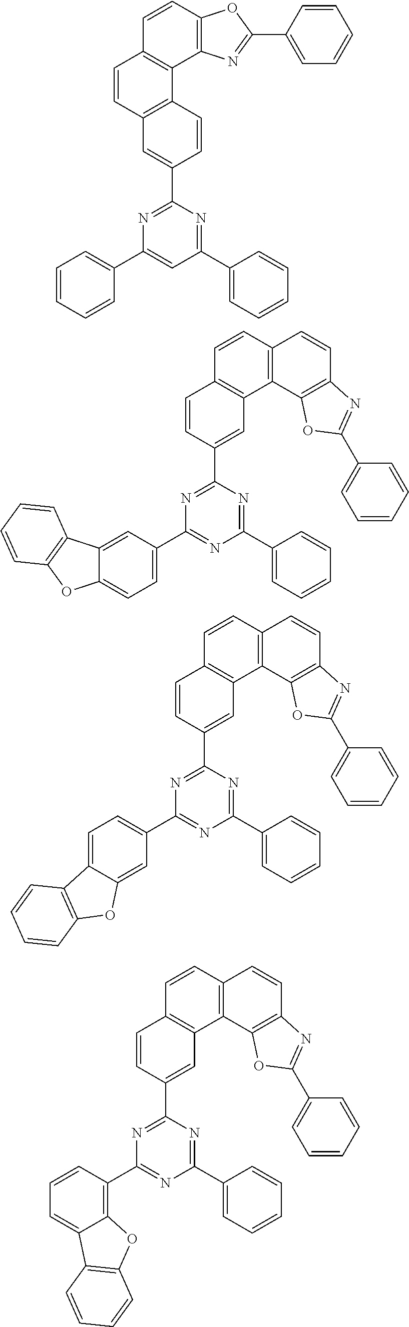

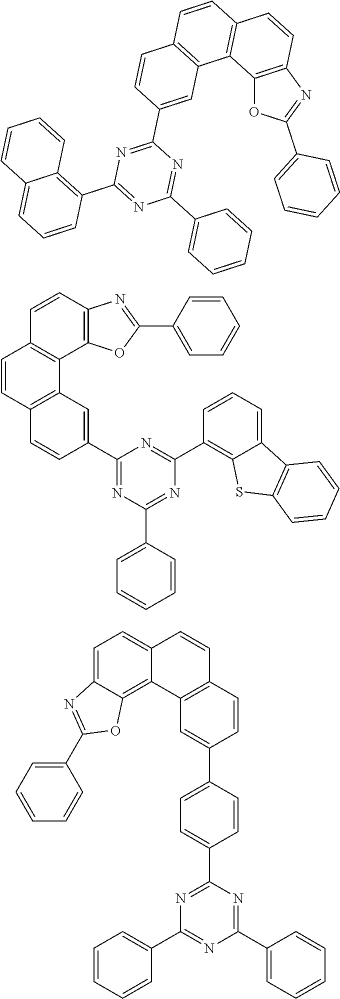

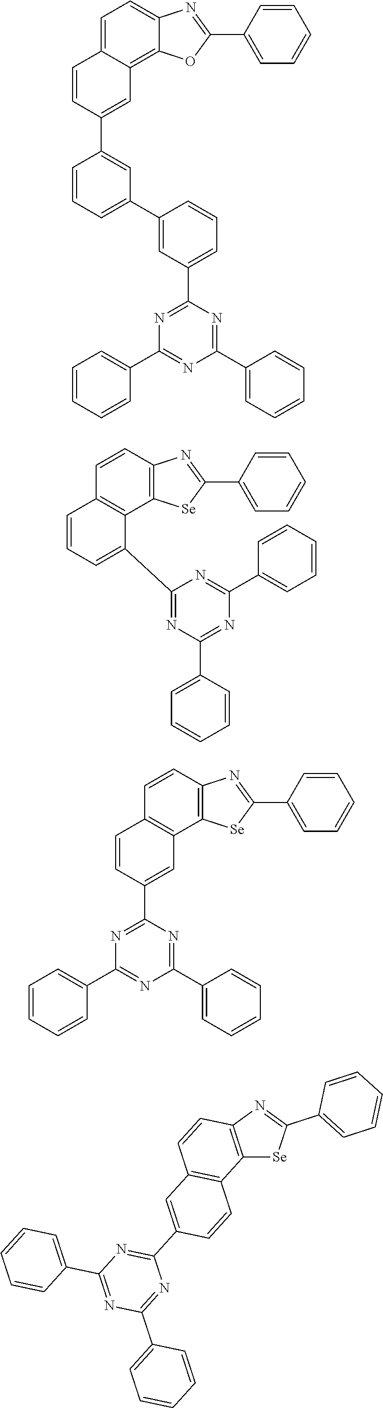

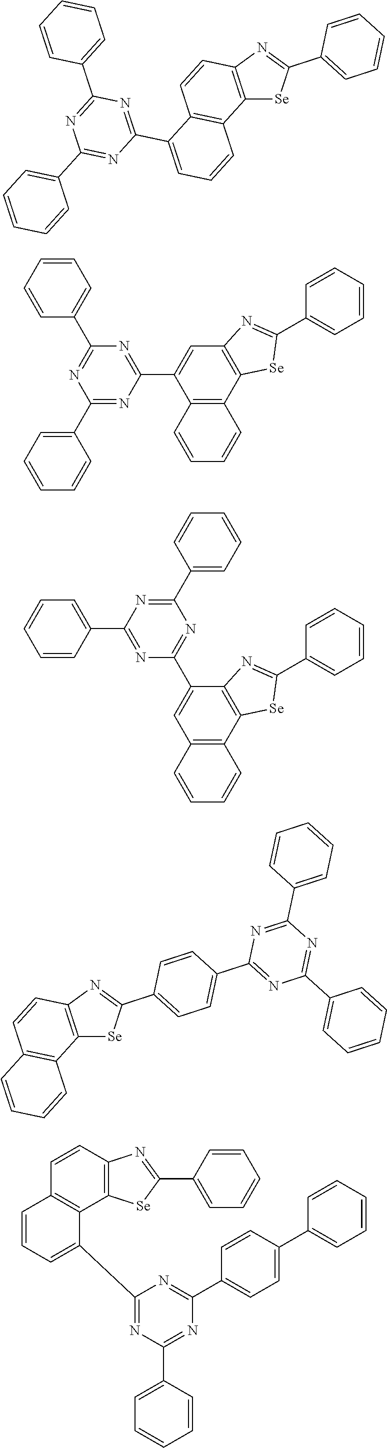

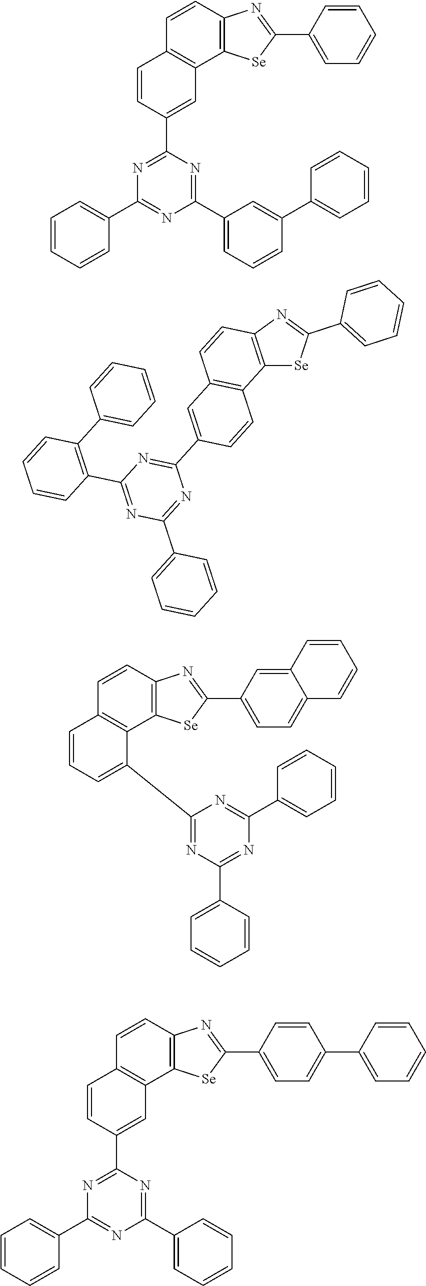 [SMILES: c1ccc(-c2cccc(-c3nc(-c4ccccc4)nc(-c4ccc5ccc6nc(-c7ccccc7)[se]c6c5c4)n3)c2)cc1 ; c1ccc(-c2nc(-c3ccc4c(ccc5nc(-c6ccccc6)[se]c54)c3)nc(-c3ccccc3-c3ccccc3)n2)cc1 ; c1ccc(-c2nc(-c3ccccc3)nc(-c3cccc4ccc5nc(-c6ccc7ccccc7c6)[se]c5c34)n2)cc1 ; c1ccc(-c2ccc(-c3nc4ccc5ccc(-c6nc(-c7ccccc7)nc(-c7ccccc7)n6)cc5c4[se]3)cc2)cc1]

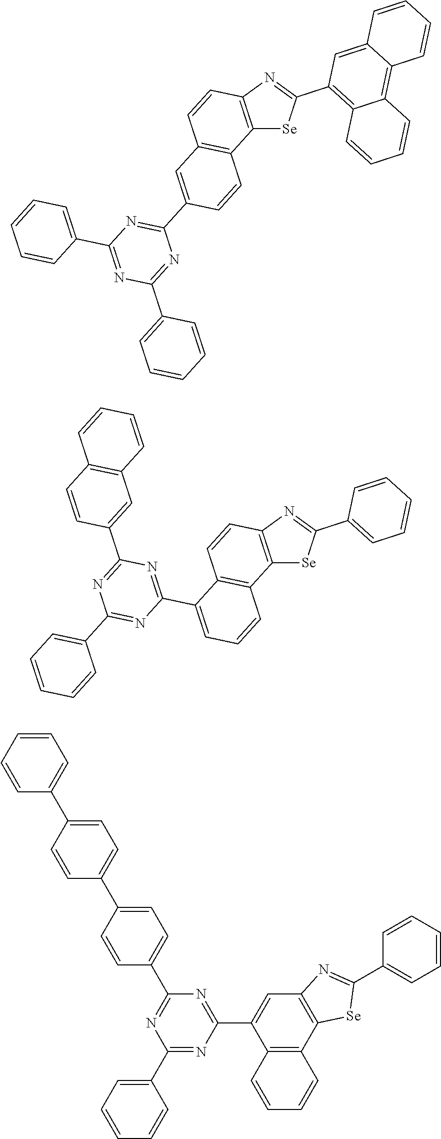

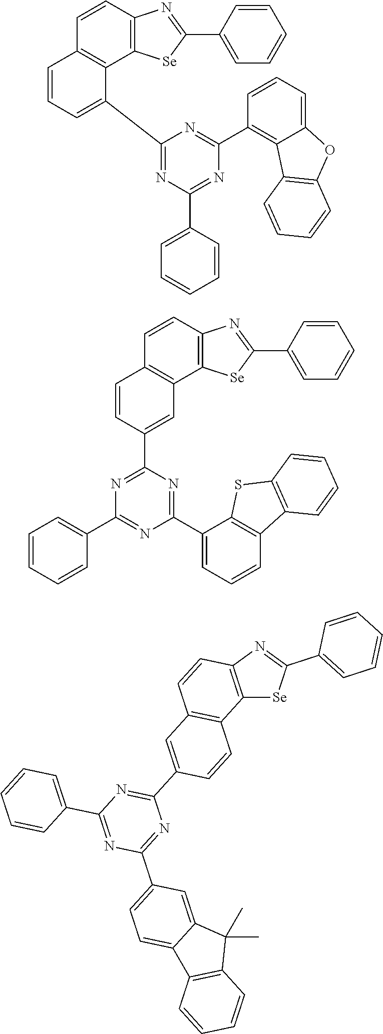

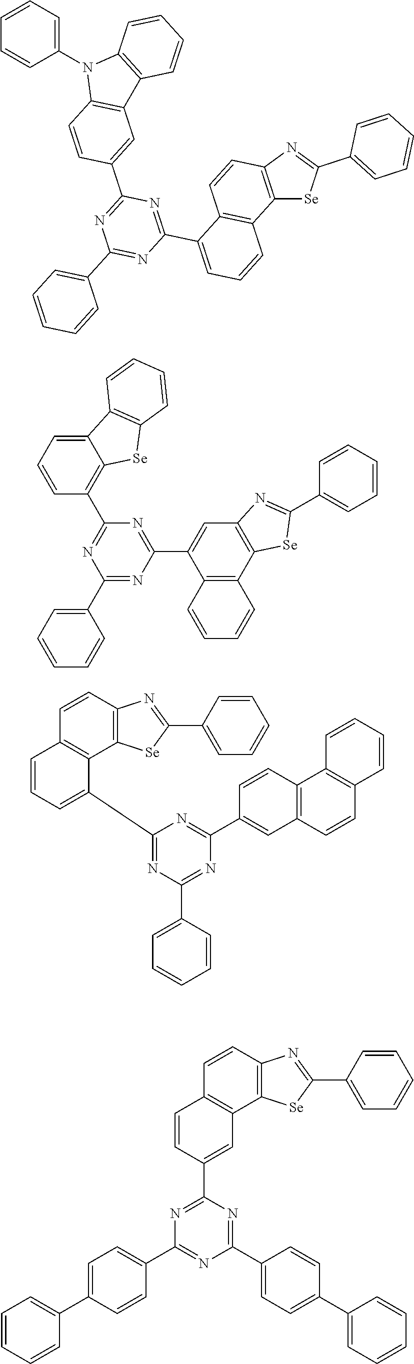

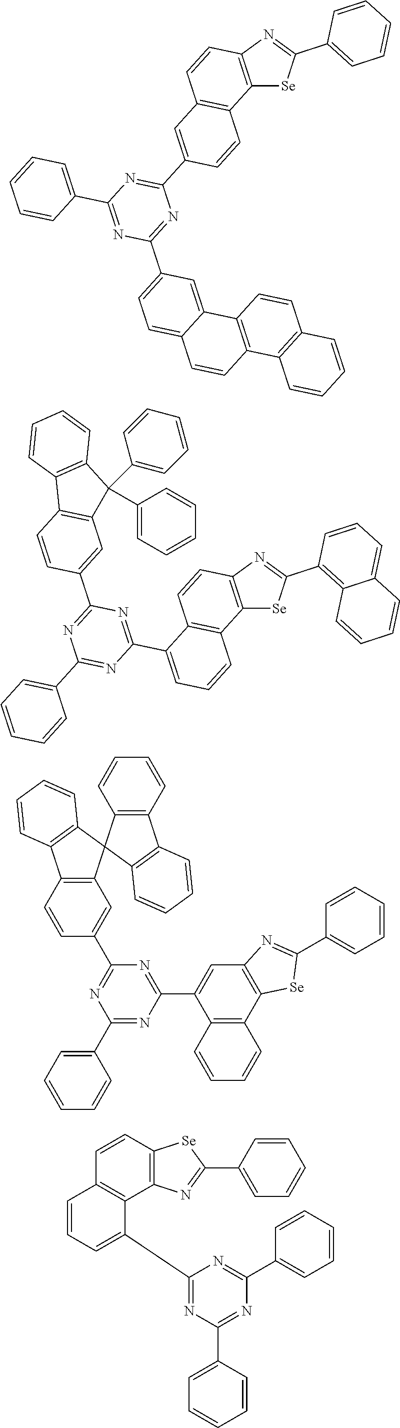

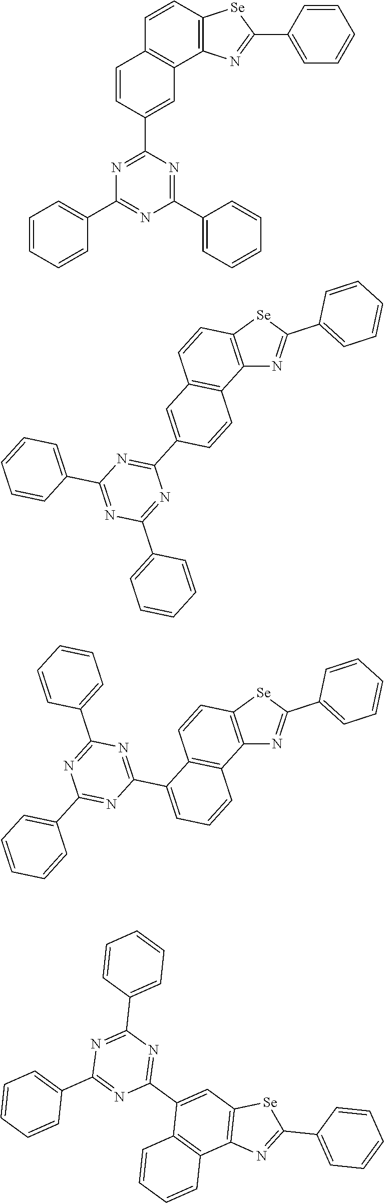 [SMILES: c1ccc(-c2nc(-c3ccccc3)nc(-c3ccc4ccc5[se]c(-c6ccccc6)nc5c4c3)n2)cc1 ; c1ccc(-c2nc(-c3ccccc3)nc(-c3ccc4c(ccc5[se]c(-c6ccccc6)nc54)c3)n2)cc1 ; c1ccc(-c2nc(-c3ccccc3)nc(-c3cccc4c3ccc3[se]c(-c5ccccc5)nc34)n2)cc1 ; c1ccc(-c2nc(-c3ccccc3)nc(-c3cc4[se]c(-c5ccccc5)nc4c4ccccc34)n2)cc1]

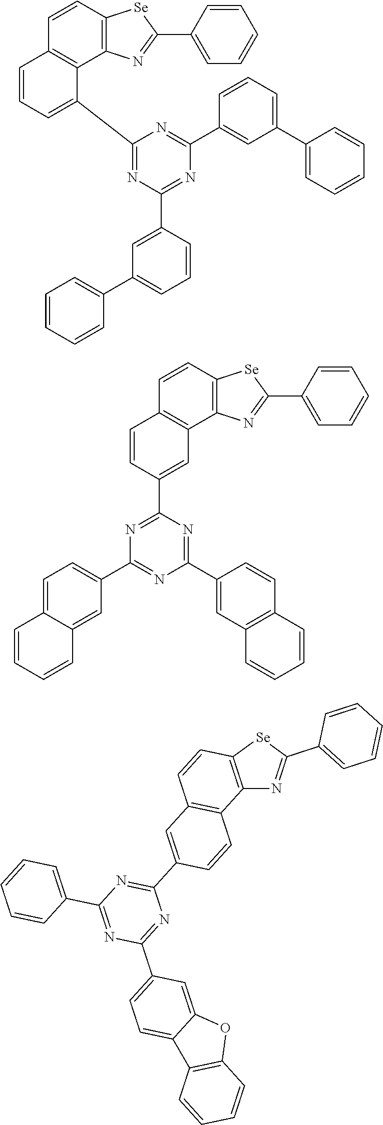 [SMILES: c1ccc(-c2cccc(-c3nc(-c4cccc(-c5ccccc5)c4)nc(-c4cccc5ccc6[se]c(-c7ccccc7)nc6c45)n3)c2)cc1 ; c1ccc(-c2nc3c(ccc4ccc(-c5nc(-c6ccc7ccccc7c6)nc(-c6ccc7ccccc7c6)n5)cc43)[se]2)cc1 ; c1ccc(-c2nc(-c3ccc4c(ccc5[se]c(-c6ccccc6)nc54)c3)nc(-c3ccc4c(c3)oc3ccccc34)n2)cc1]

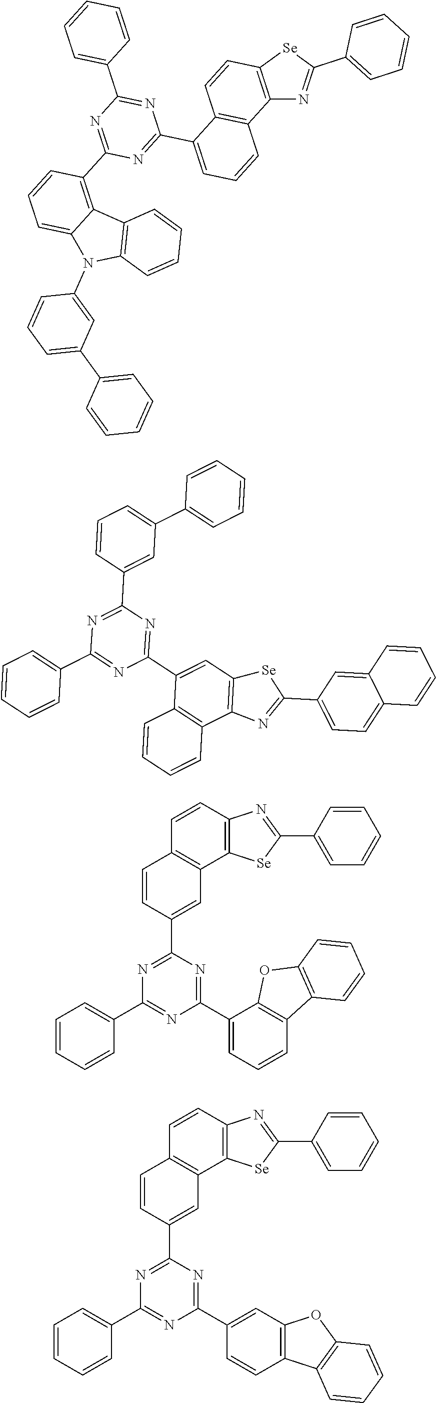

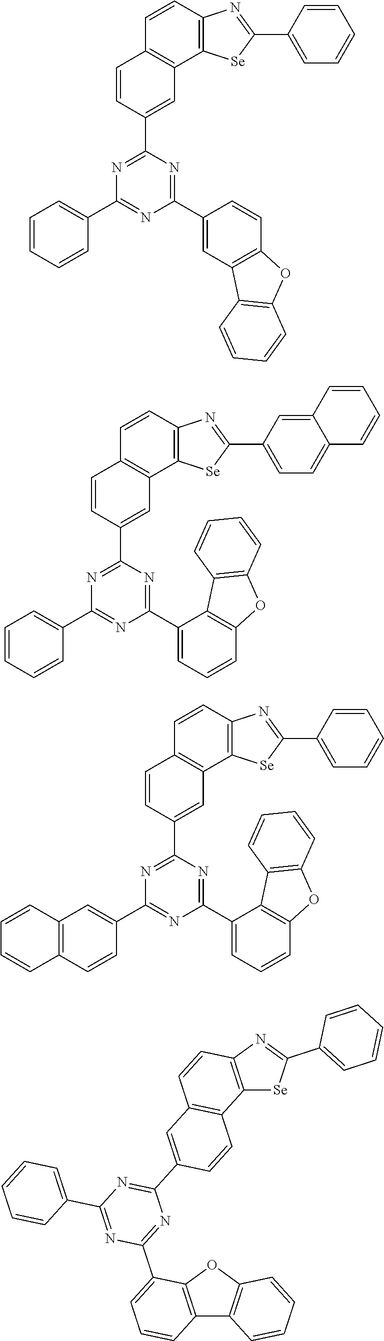

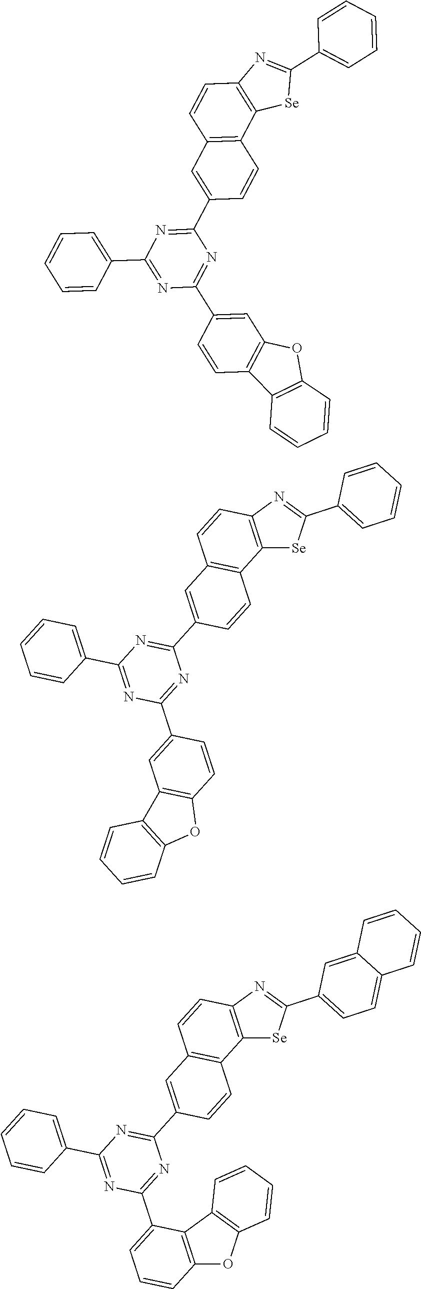

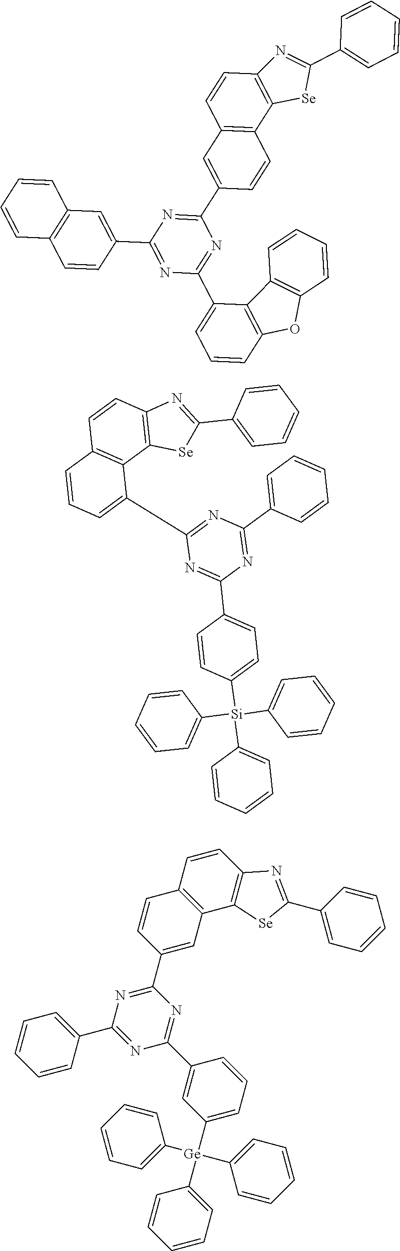

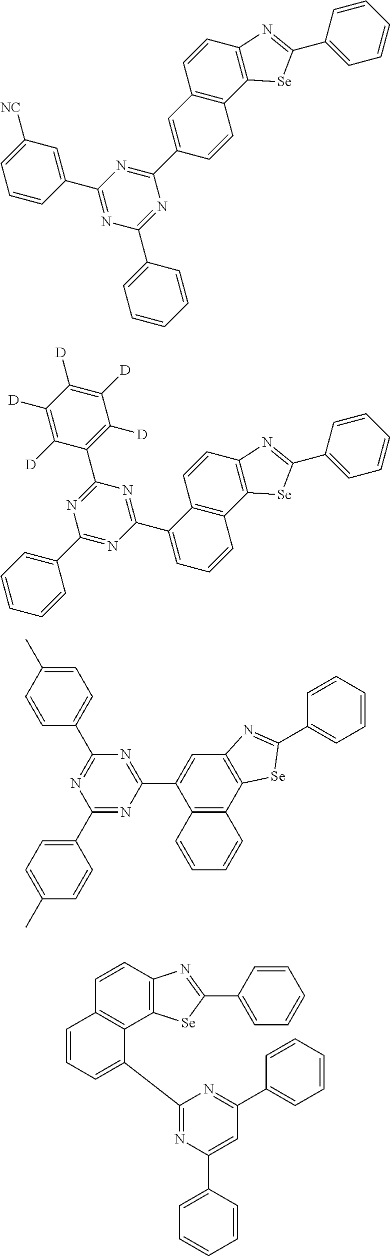

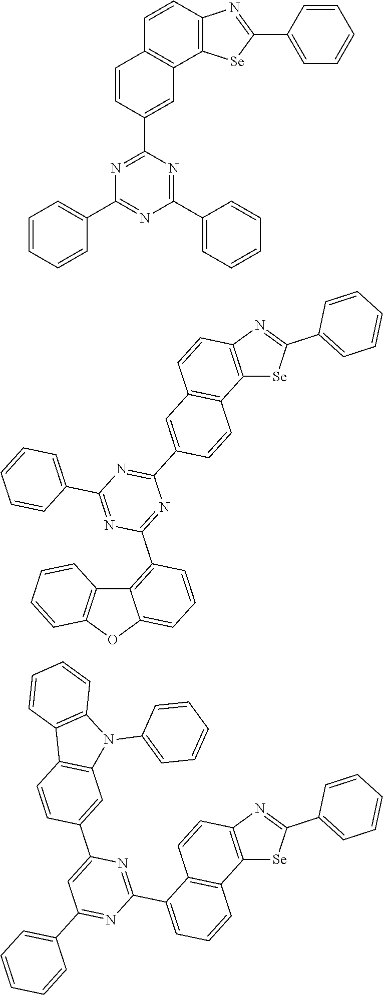

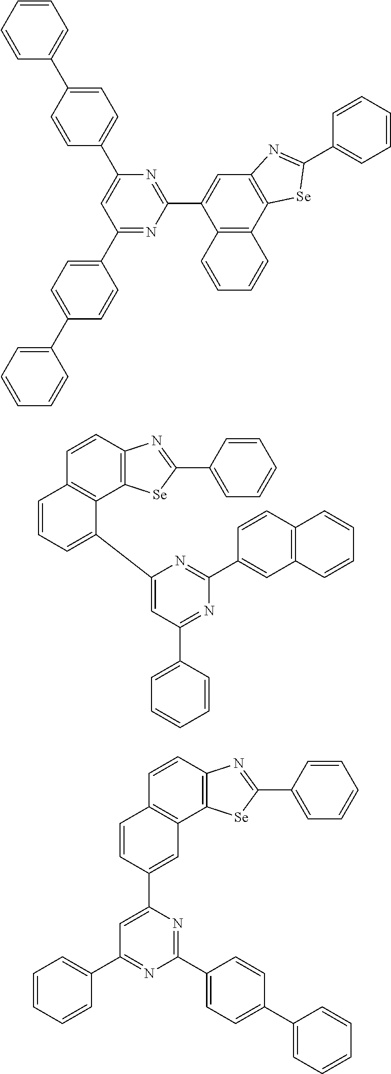

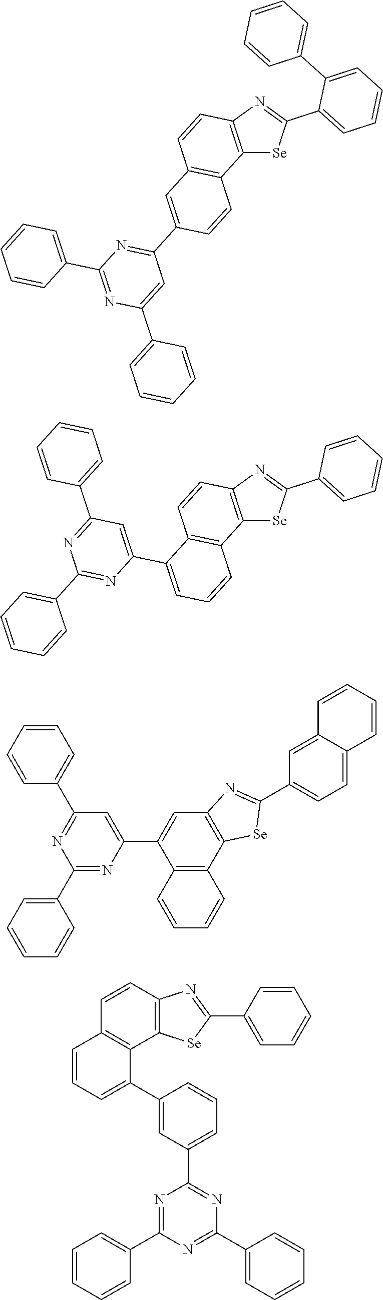 [SMILES: c1ccc(-c2cc(-c3ccc4c(ccc5nc(-c6ccccc6-c6ccccc6)[se]c54)c3)nc(-c3ccccc3)n2)cc1 ; c1ccc(-c2cc(-c3cccc4c3ccc3nc(-c5ccccc5)[se]c34)nc(-c3ccccc3)n2)cc1 ; c1ccc(-c2cc(-c3cc4nc(-c5ccc6ccccc6c5)[se]c4c4ccccc34)nc(-c3ccccc3)n2)cc1 ; c1ccc(-c2nc(-c3ccccc3)nc(-c3cccc(-c4cccc5ccc6nc(-c7ccccc7)[se]c6c45)c3)n2)cc1]

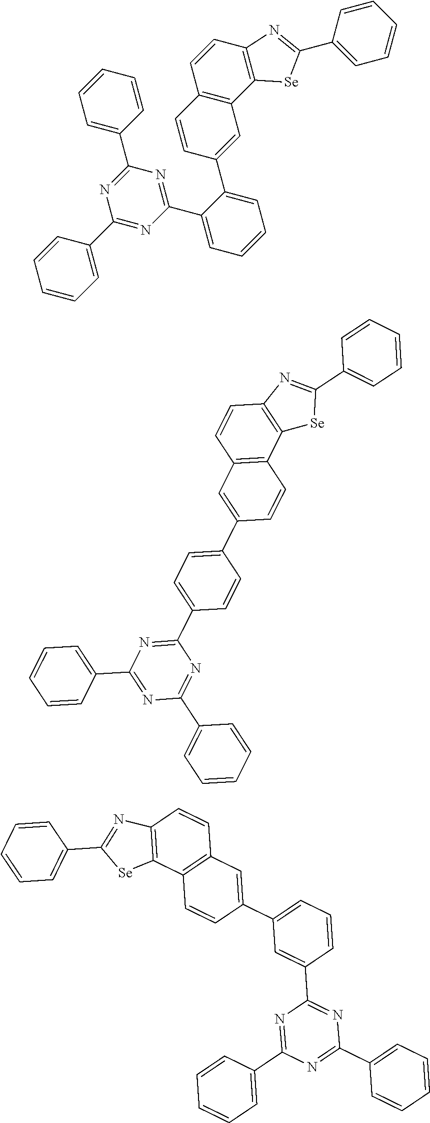

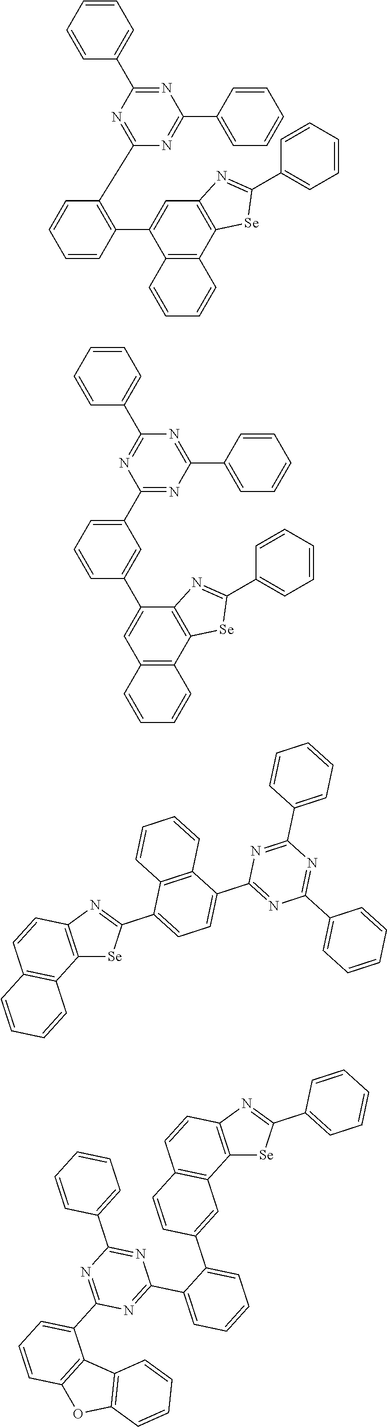 [SMILES: c1ccc(-c2nc(-c3ccccc3)nc(-c3ccccc3-c3cc4nc(-c5ccccc5)[se]c4c4ccccc34)n2)cc1 ; c1ccc(-c2nc(-c3ccccc3)nc(-c3cccc(-c4cc5ccccc5c5[se]c(-c6ccccc6)nc45)c3)n2)cc1 ; c1ccc(-c2nc(-c3ccccc3)nc(-c3ccc(-c4nc5ccc6ccccc6c5[se]4)c4ccccc34)n2)cc1 ; c1ccc(-c2nc(-c3ccccc3-c3ccc4ccc5nc(-c6ccccc6)[se]c5c4c3)nc(-c3cccc4oc5ccccc5c34)n2)cc1]

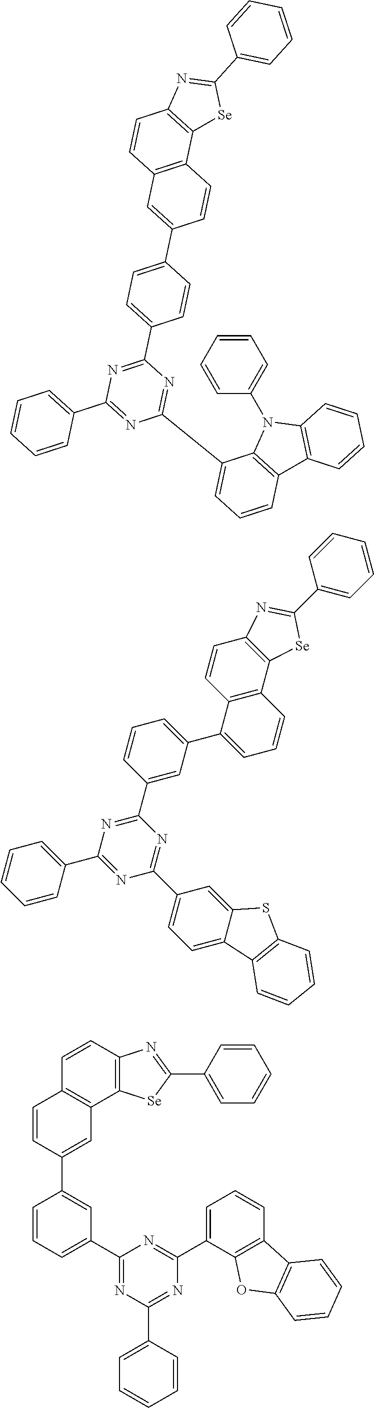 [SMILES: c1ccc(-c2nc(-c3ccc(-c4ccc5c(ccc6nc(-c7ccccc7)[se]c65)c4)cc3)nc(-c3cccc4c5ccccc5n(-c5ccccc5)c34)n2)cc1 ; c1ccc(-c2nc(-c3cccc(-c4cccc5c4ccc4nc(-c6ccccc6)[se]c45)c3)nc(-c3ccc4c(c3)sc3ccccc34)n2)cc1 ; c1ccc(-c2nc(-c3cccc(-c4ccc5ccc6nc(-c7ccccc7)[se]c6c5c4)c3)nc(-c3cccc4c3oc3ccccc34)n2)cc1]

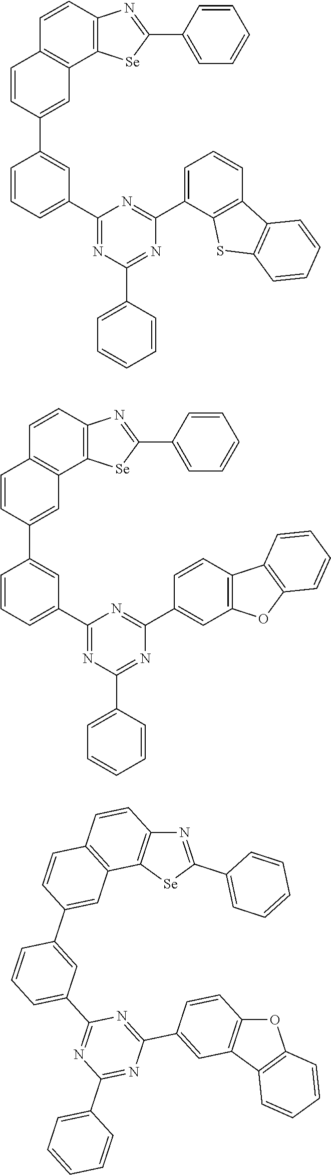

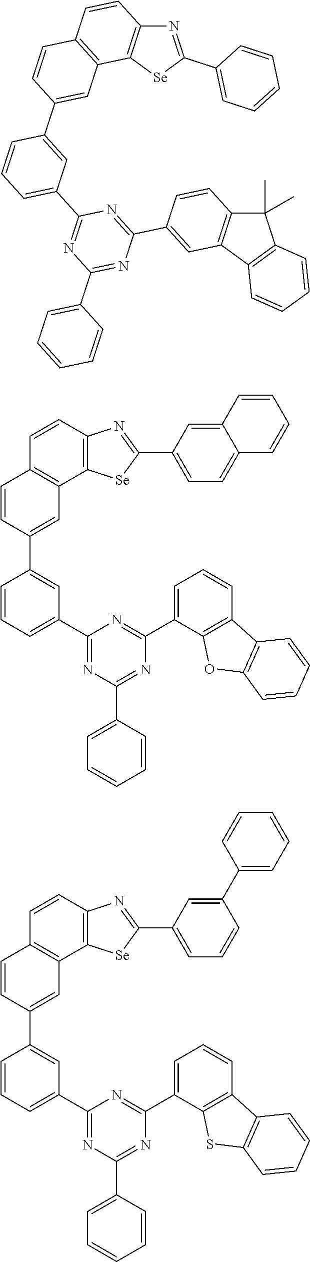 [SMILES: CC1(C)c2ccccc2-c2cc(-c3nc(-c4ccccc4)nc(-c4cccc(-c5ccc6ccc7nc(-c8ccccc8)[se]c7c6c5)c4)n3)ccc21 ; c1ccc(-c2nc(-c3cccc(-c4ccc5ccc6nc(-c7ccc8ccccc8c7)[se]c6c5c4)c3)nc(-c3cccc4c3oc3ccccc34)n2)cc1 ; c1ccc(-c2cccc(-c3nc4ccc5ccc(-c6cccc(-c7nc(-c8ccccc8)nc(-c8cccc9c8sc8ccccc89)n7)c6)cc5c4[se]3)c2)cc1]

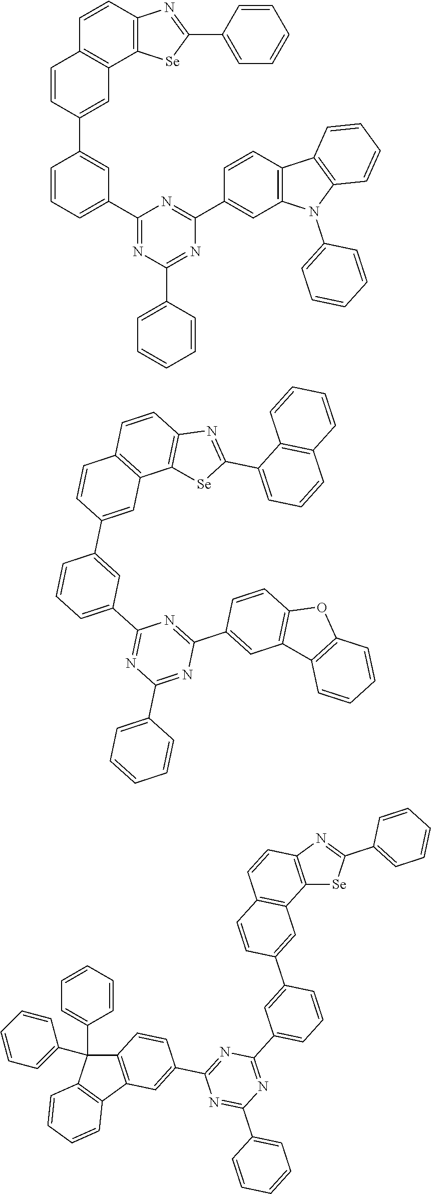

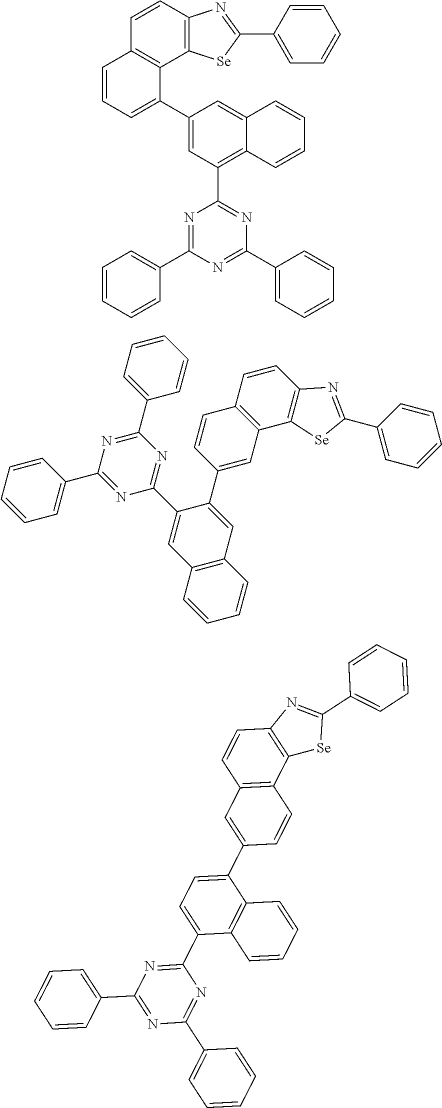

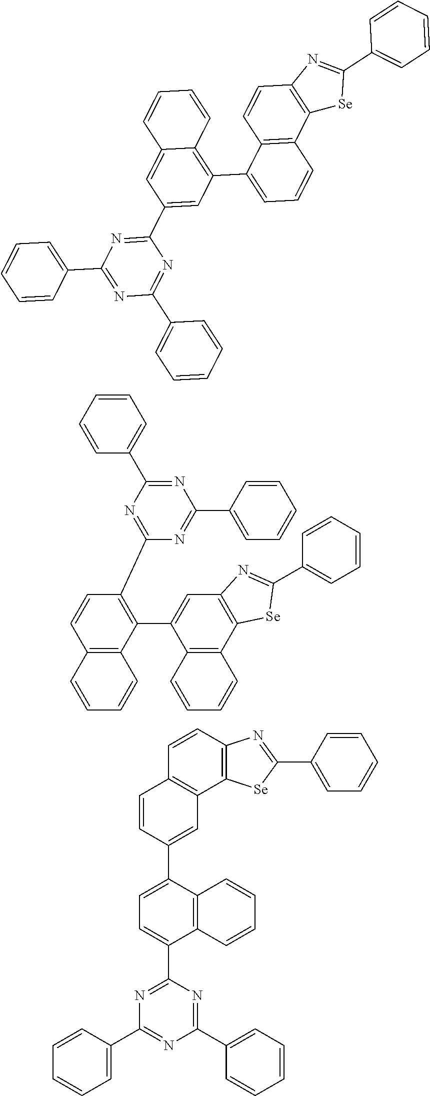

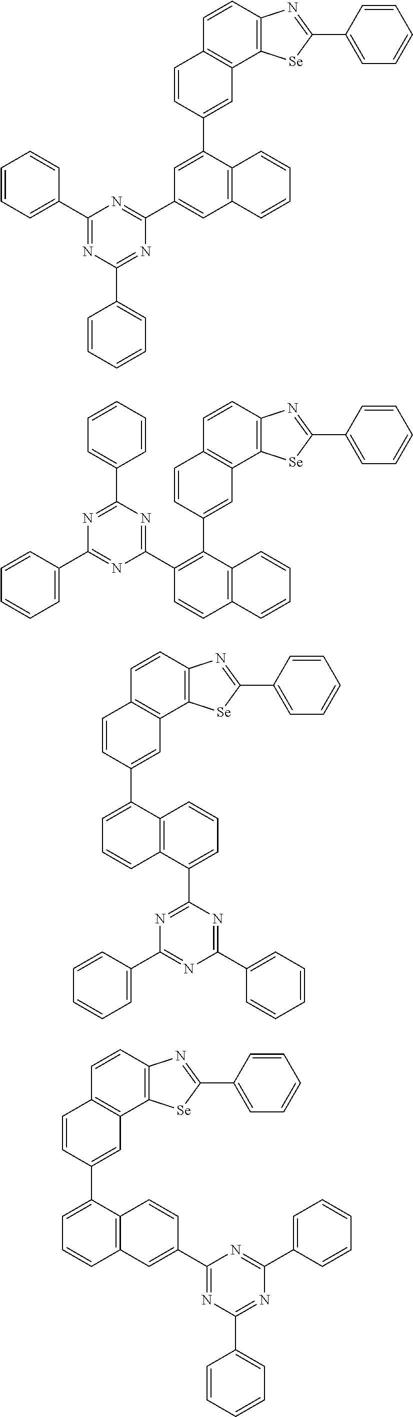

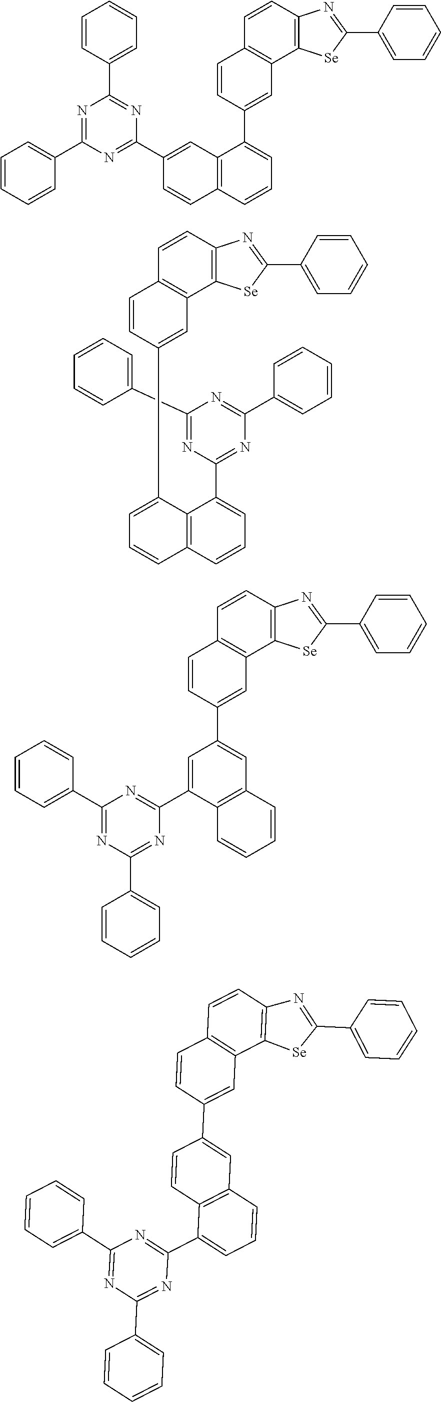

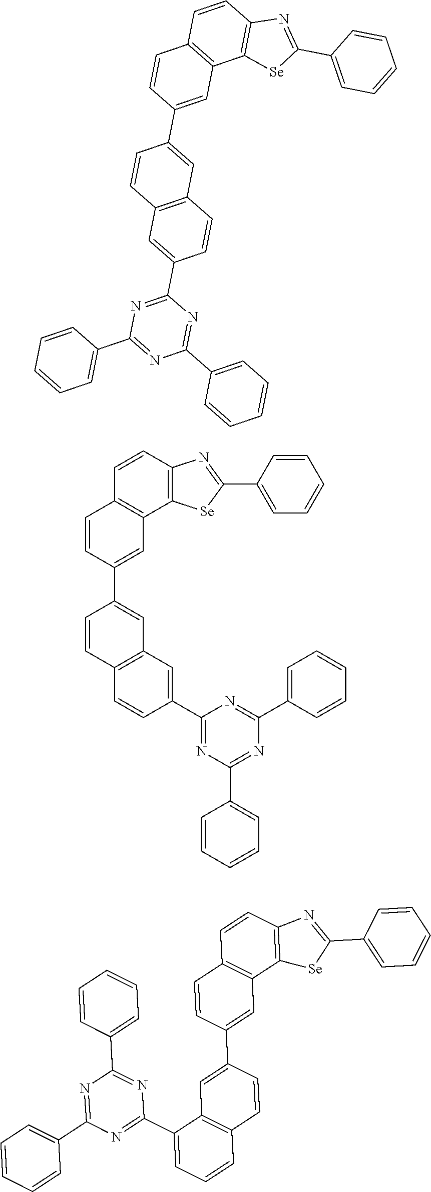

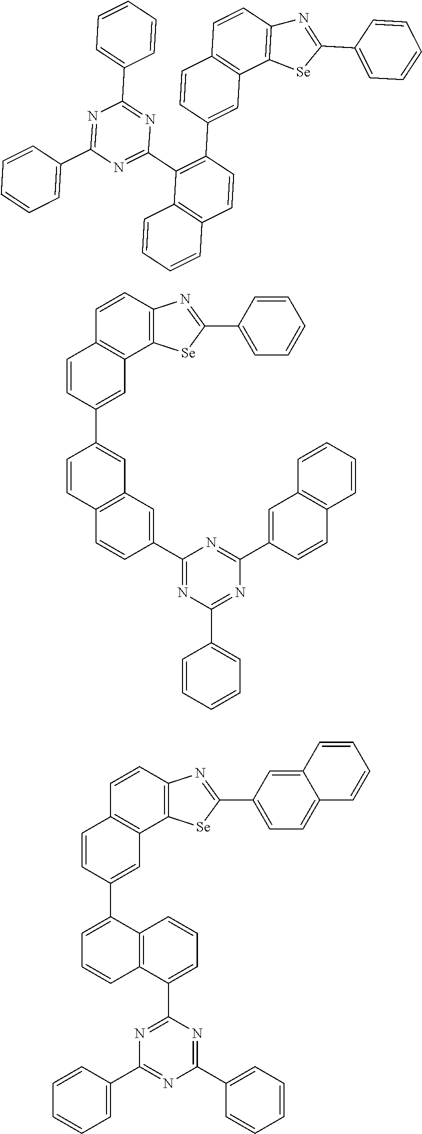

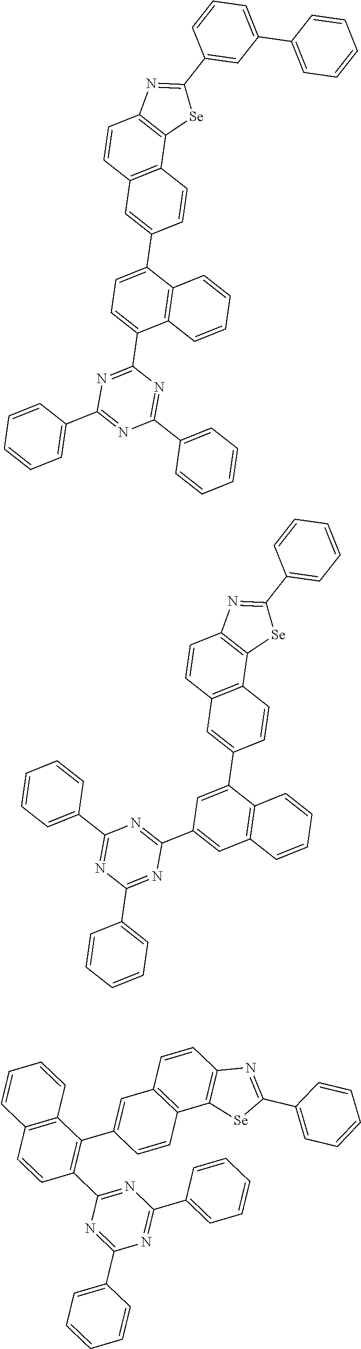 [SMILES: c1ccc(-c2cccc(-c3nc4ccc5cc(-c6ccc(-c7nc(-c8ccccc8)nc(-c8ccccc8)n7)c7ccccc67)ccc5c4[se]3)c2)cc1 ; c1ccc(-c2nc(-c3ccccc3)nc(-c3cc(-c4ccc5c(ccc6nc(-c7ccccc7)[se]c65)c4)c4ccccc4c3)n2)cc1 ; c1ccc(-c2nc(-c3ccccc3)nc(-c3ccc4ccccc4c3-c3ccc4c(ccc5nc(-c6ccccc6)[se]c54)c3)n2)cc1]

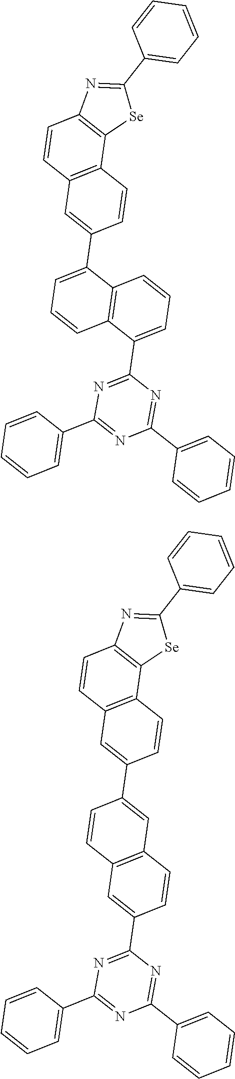

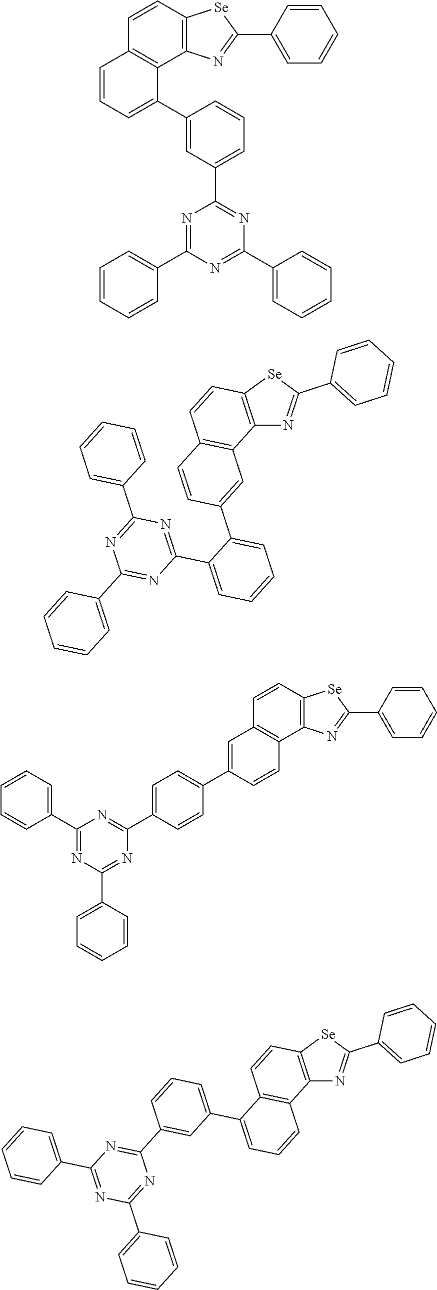 [SMILES: c1ccc(-c2nc(-c3ccccc3)nc(-c3cccc(-c4cccc5ccc6[se]c(-c7ccccc7)nc6c45)c3)n2)cc1 ; c1ccc(-c2nc(-c3ccccc3)nc(-c3ccccc3-c3ccc4ccc5[se]c(-c6ccccc6)nc5c4c3)n2)cc1 ; c1ccc(-c2nc(-c3ccccc3)nc(-c3ccc(-c4ccc5c(ccc6[se]c(-c7ccccc7)nc65)c4)cc3)n2)cc1 ; c1ccc(-c2nc(-c3ccccc3)nc(-c3cccc(-c4cccc5c4ccc4[se]c(-c6ccccc6)nc45)c3)n2)cc1]

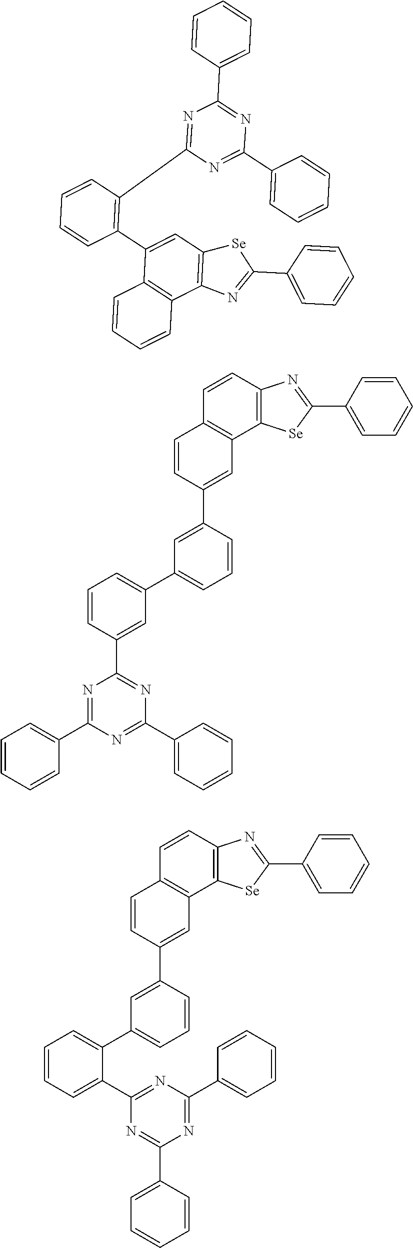

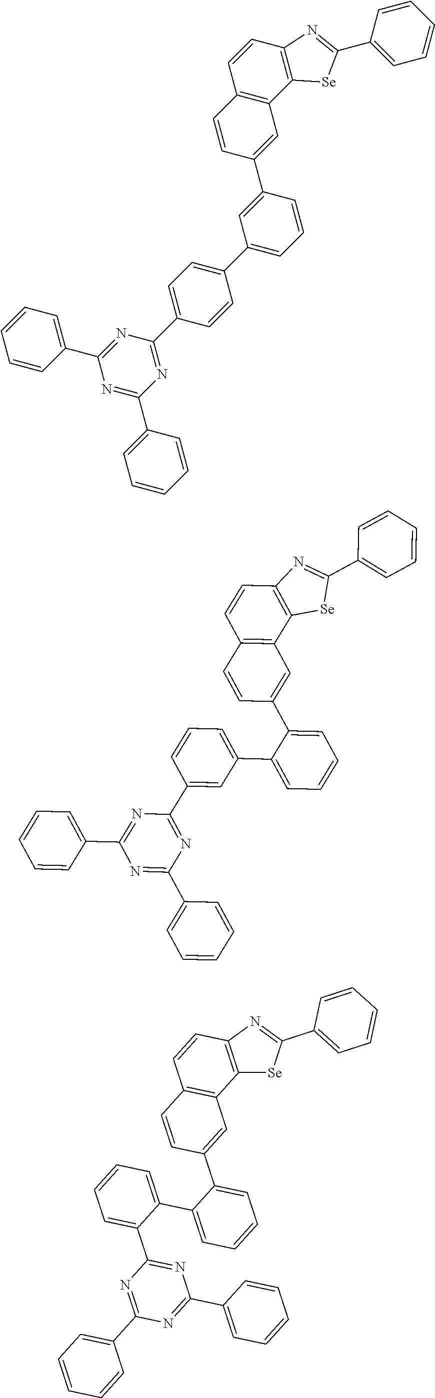 [SMILES: c1ccc(-c2nc(-c3ccccc3)nc(-c3ccc(-c4cccc(-c5ccc6ccc7nc(-c8ccccc8)[se]c7c6c5)c4)cc3)n2)cc1 ; c1ccc(-c2nc(-c3ccccc3)nc(-c3cccc(-c4ccccc4-c4ccc5ccc6nc(-c7ccccc7)[se]c6c5c4)c3)n2)cc1 ; c1ccc(-c2nc(-c3ccccc3)nc(-c3ccccc3-c3ccccc3-c3ccc4ccc5nc(-c6ccccc6)[se]c5c4c3)n2)cc1]

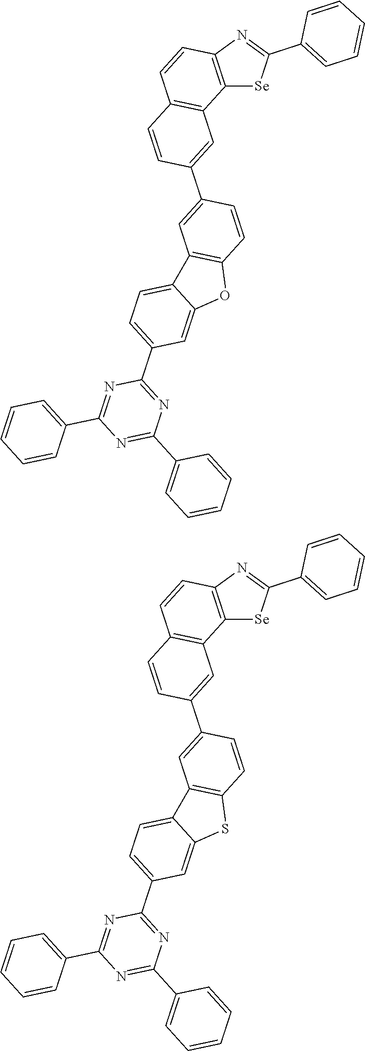 [SMILES: c1ccc(-c2nc(-c3ccccc3)nc(-c3ccc4c(c3)oc3ccc(-c5ccc6ccc7nc(-c8ccccc8)[se]c7c6c5)cc34)n2)cc1 ; c1ccc(-c2nc(-c3ccccc3)nc(-c3ccc4c(c3)sc3ccc(-c5ccc6ccc7nc(-c8ccccc8)[se]c7c6c5)cc34)n2)cc1]

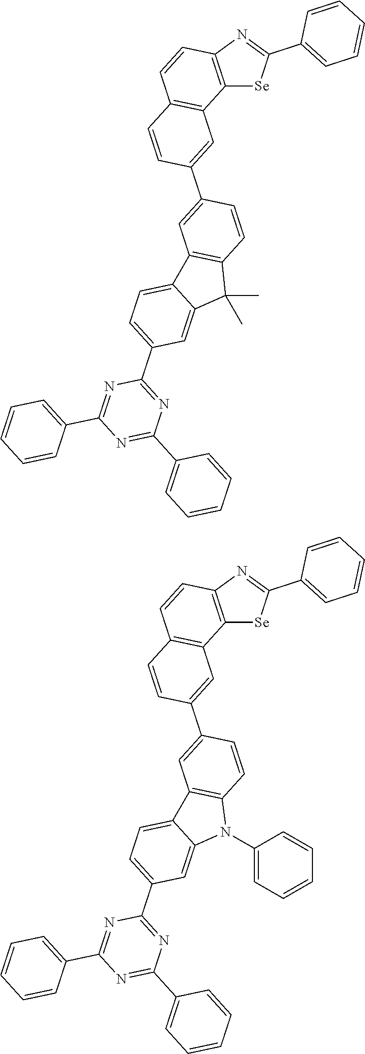 [SMILES: CC1(C)c2ccc(-c3ccc4ccc5nc(-c6ccccc6)[se]c5c4c3)cc2-c2ccc(-c3nc(-c4ccccc4)nc(-c4ccccc4)n3)cc21 ; c1ccc(-c2nc(-c3ccccc3)nc(-c3ccc4c5cc(-c6ccc7ccc8nc(-c9ccccc9)[se]c8c7c6)ccc5n(-c5ccccc5)c4c3)n2)cc1]

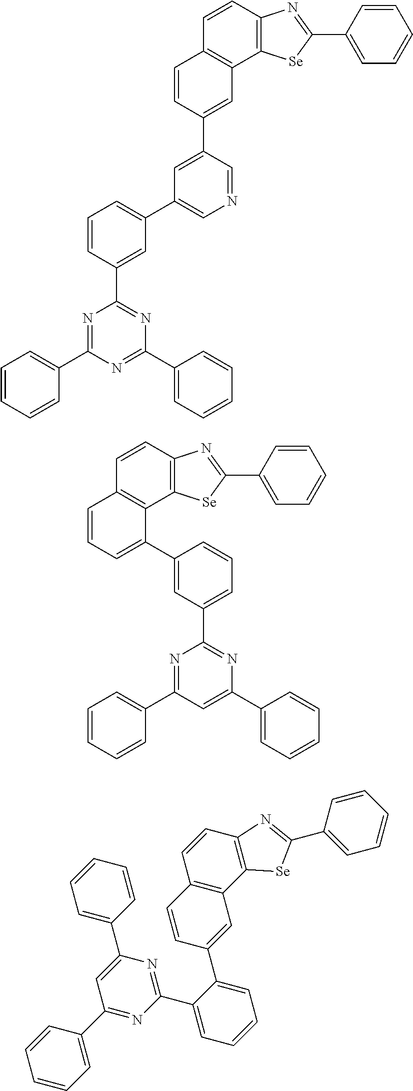

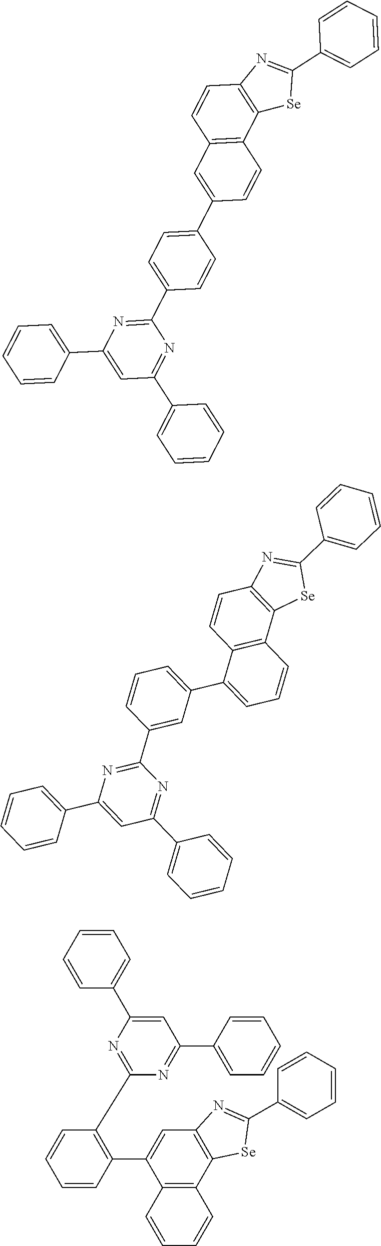 [SMILES: c1ccc(-c2cc(-c3ccccc3)nc(-c3ccc(-c4ccc5c(ccc6nc(-c7ccccc7)[se]c65)c4)cc3)n2)cc1 ; c1ccc(-c2cc(-c3ccccc3)nc(-c3cccc(-c4cccc5c4ccc4nc(-c6ccccc6)[se]c45)c3)n2)cc1 ; c1ccc(-c2cc(-c3ccccc3)nc(-c3ccccc3-c3cc4nc(-c5ccccc5)[se]c4c4ccccc34)n2)cc1]

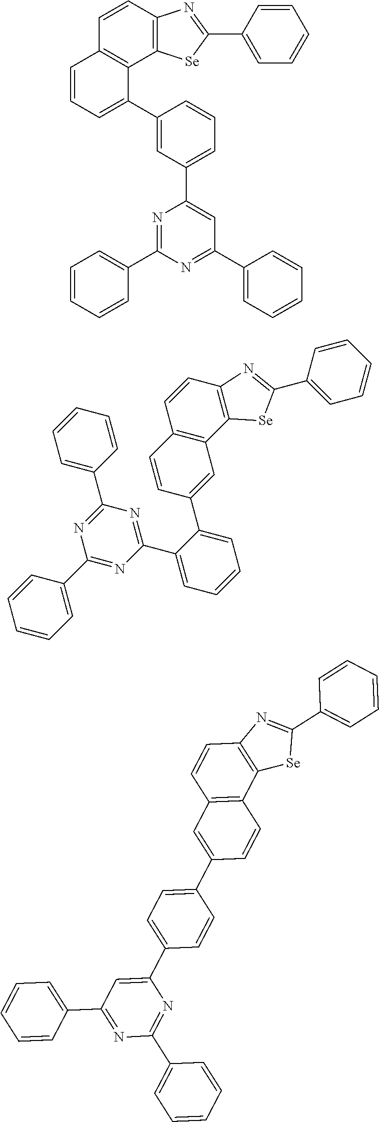 [SMILES: c1ccc(-c2cc(-c3cccc(-c4cccc5ccc6nc(-c7ccccc7)[se]c6c45)c3)nc(-c3ccccc3)n2)cc1 ; c1ccc(-c2nc(-c3ccccc3)nc(-c3ccccc3-c3ccc4ccc5nc(-c6ccccc6)[se]c5c4c3)n2)cc1 ; c1ccc(-c2cc(-c3ccc(-c4ccc5c(ccc6nc(-c7ccccc7)[se]c65)c4)cc3)nc(-c3ccccc3)n2)cc1]

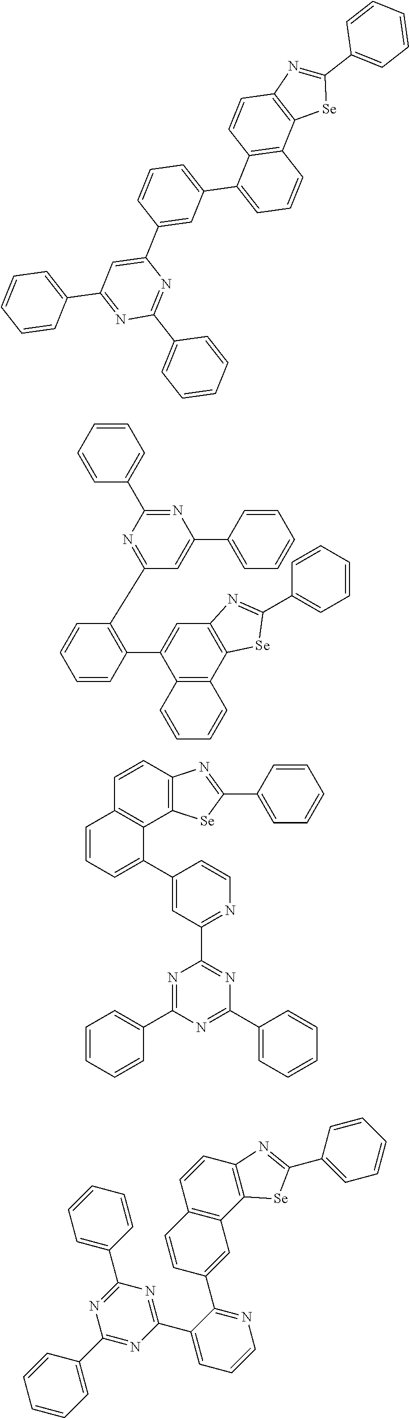

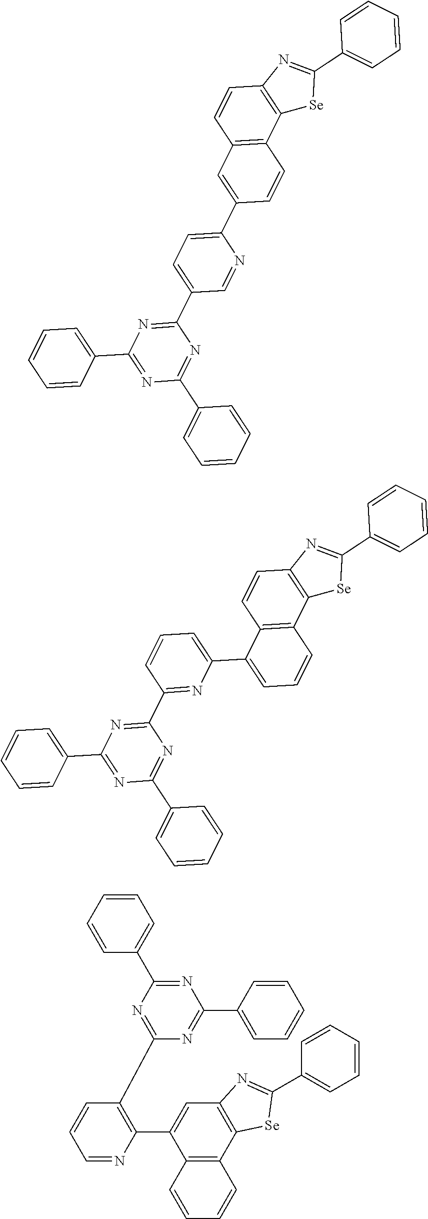 [SMILES: c1ccc(-c2nc(-c3ccccc3)nc(-c3ccc(-c4ccc5c(ccc6nc(-c7ccccc7)[se]c65)c4)nc3)n2)cc1 ; c1ccc(-c2nc(-c3ccccc3)nc(-c3cccc(-c4cccc5c4ccc4nc(-c6ccccc6)[se]c45)n3)n2)cc1 ; c1ccc(-c2nc(-c3ccccc3)nc(-c3cccnc3-c3cc4nc(-c5ccccc5)[se]c4c4ccccc34)n2)cc1]

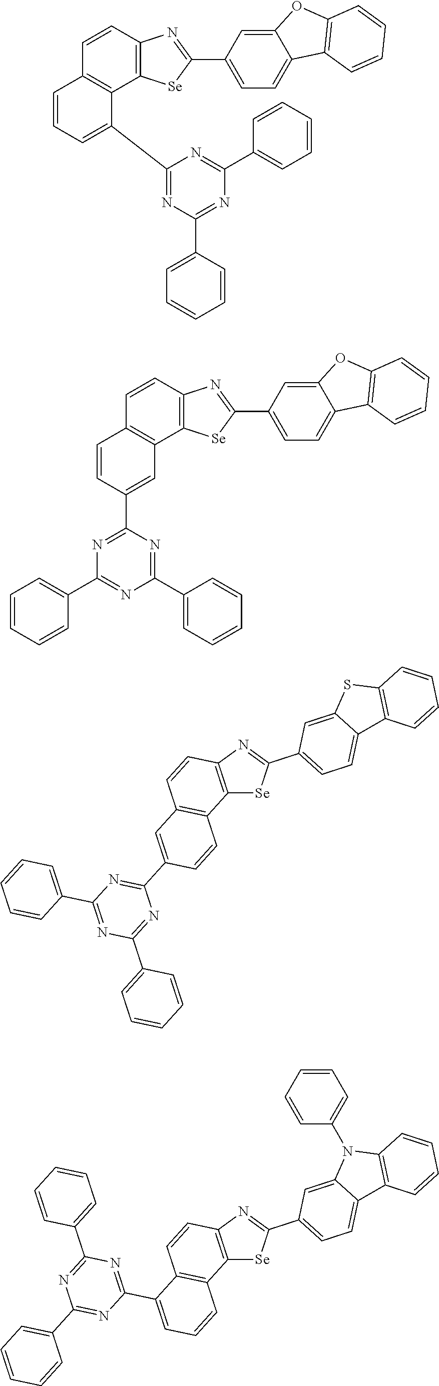 [SMILES: c1ccc(-c2nc(-c3ccccc3)nc(-c3cccc4ccc5nc(-c6ccc7c(c6)oc6ccccc67)[se]c5c34)n2)cc1 ; c1ccc(-c2nc(-c3ccccc3)nc(-c3ccc4ccc5nc(-c6ccc7c(c6)oc6ccccc67)[se]c5c4c3)n2)cc1 ; c1ccc(-c2nc(-c3ccccc3)nc(-c3ccc4c(ccc5nc(-c6ccc7c(c6)sc6ccccc67)[se]c54)c3)n2)cc1 ; c1ccc(-c2nc(-c3ccccc3)nc(-c3cccc4c3ccc3nc(-c5ccc6c7ccccc7n(-c7ccccc7)c6c5)[se]c34)n2)cc1]

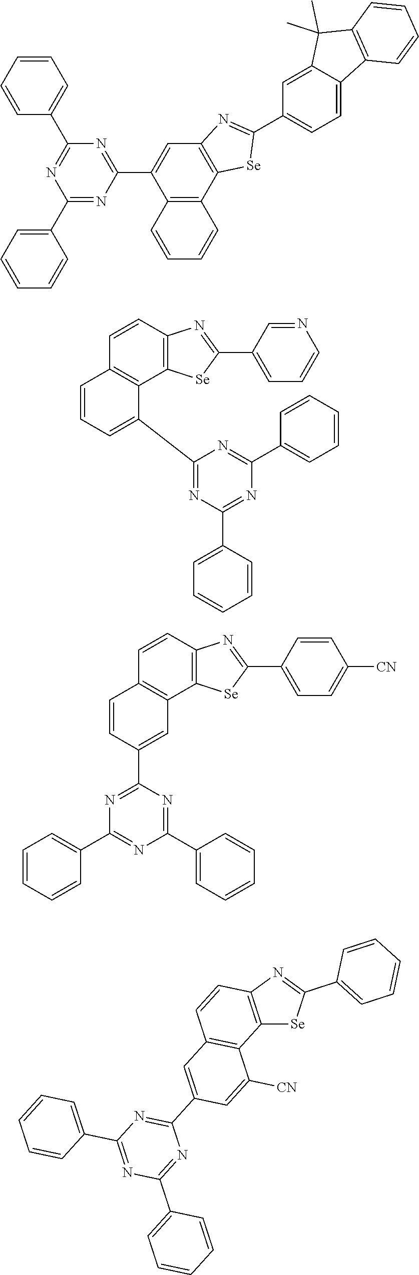

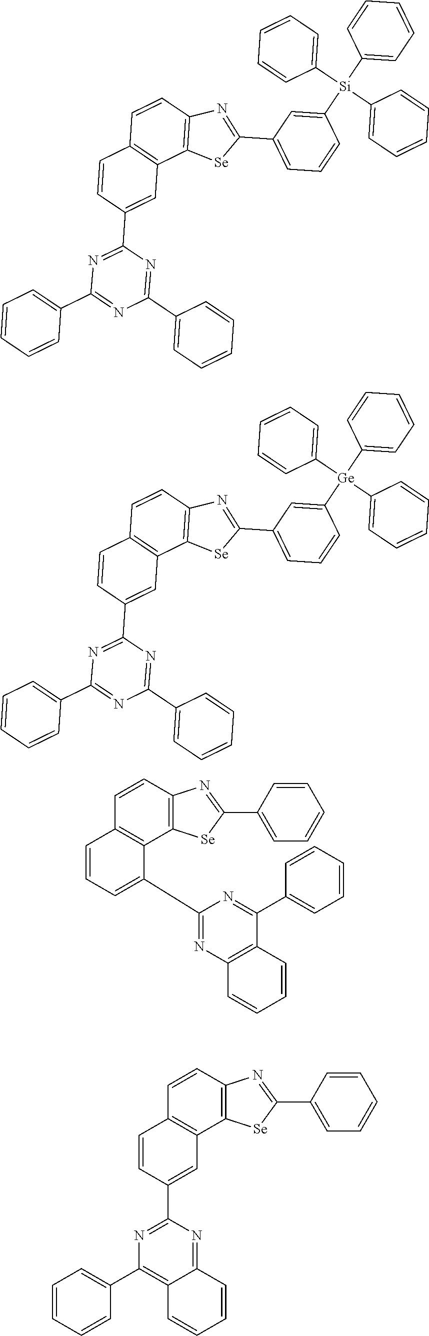

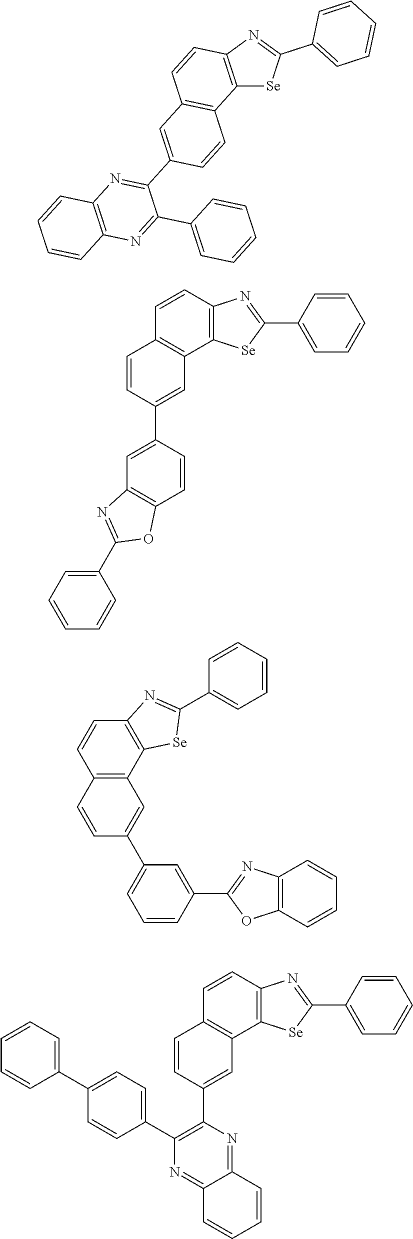

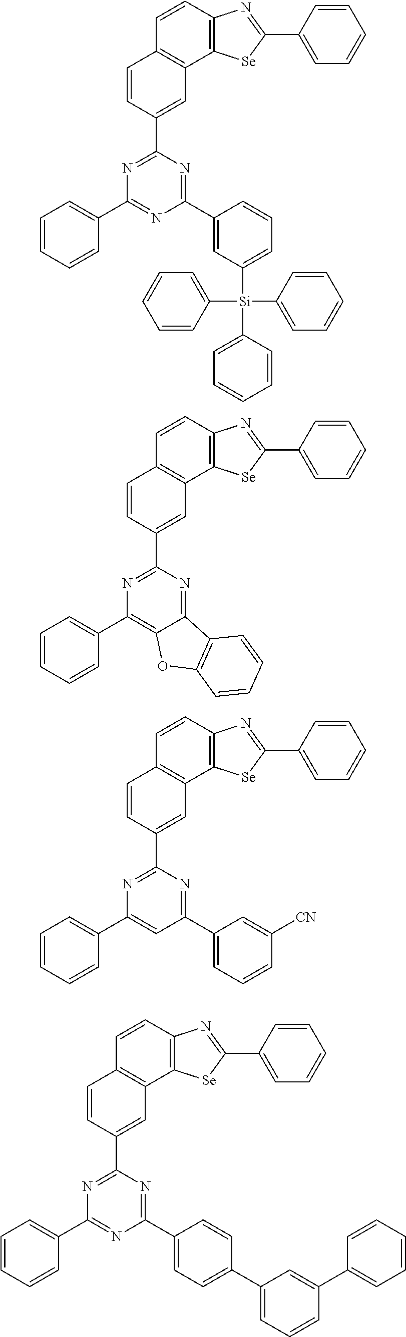

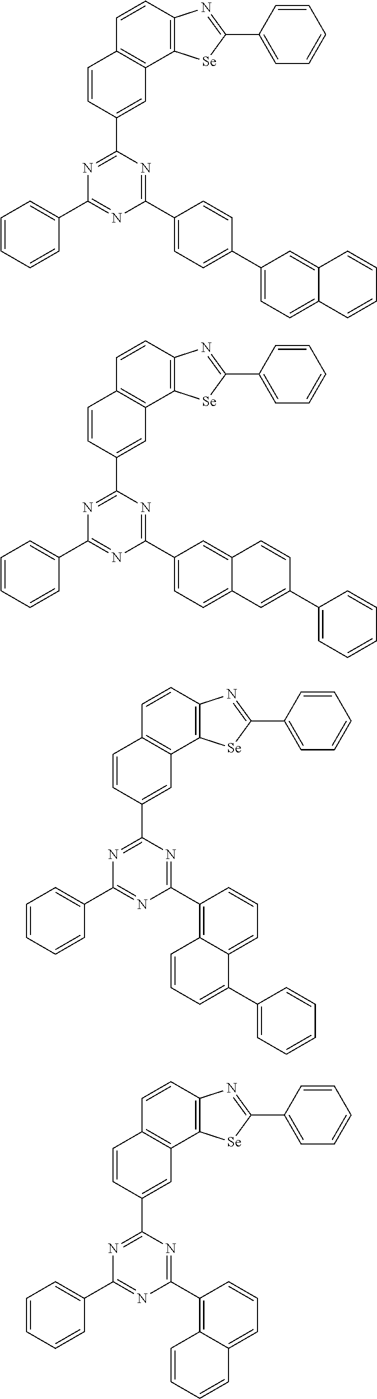

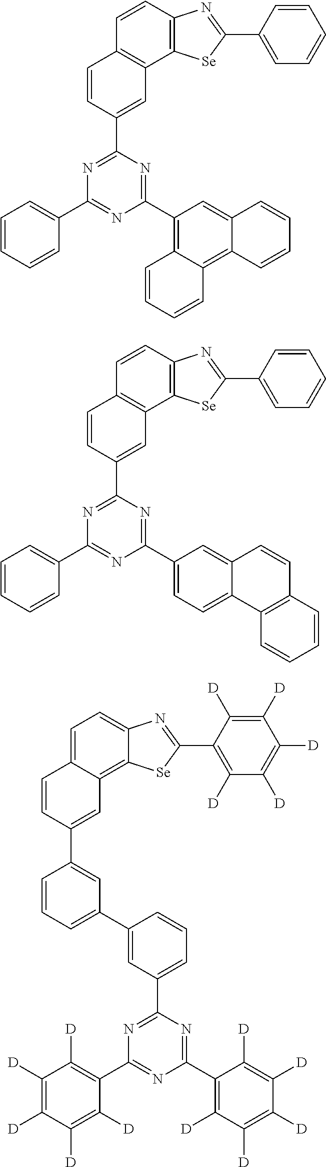

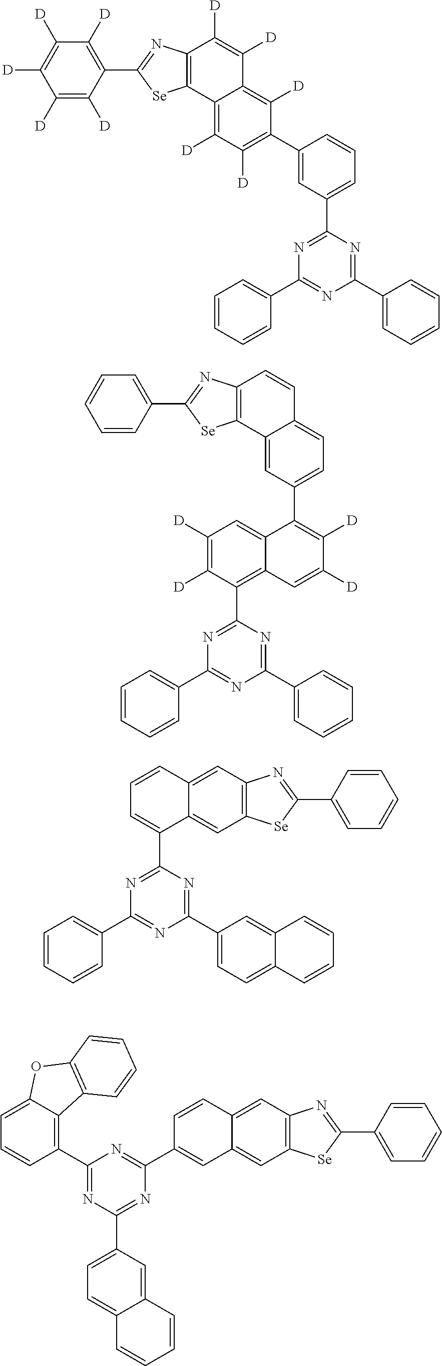

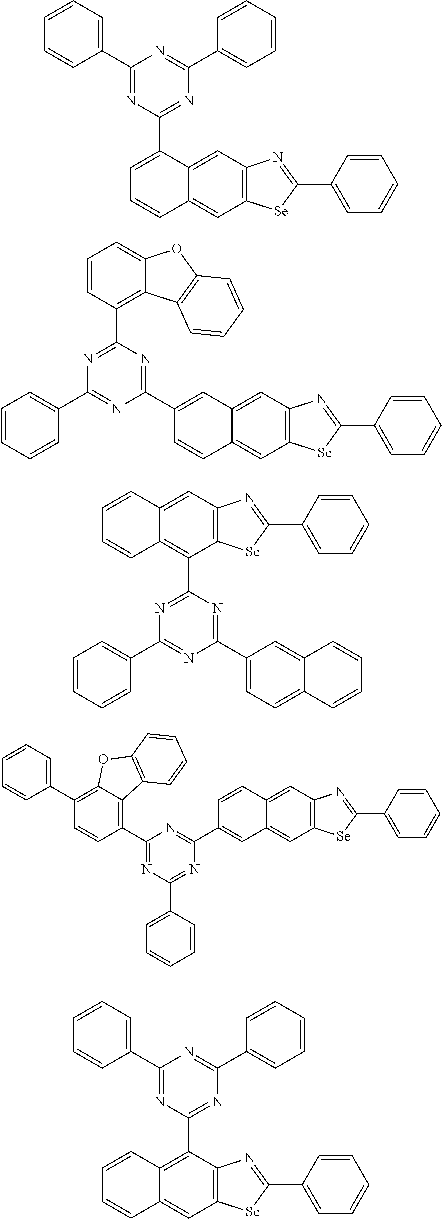

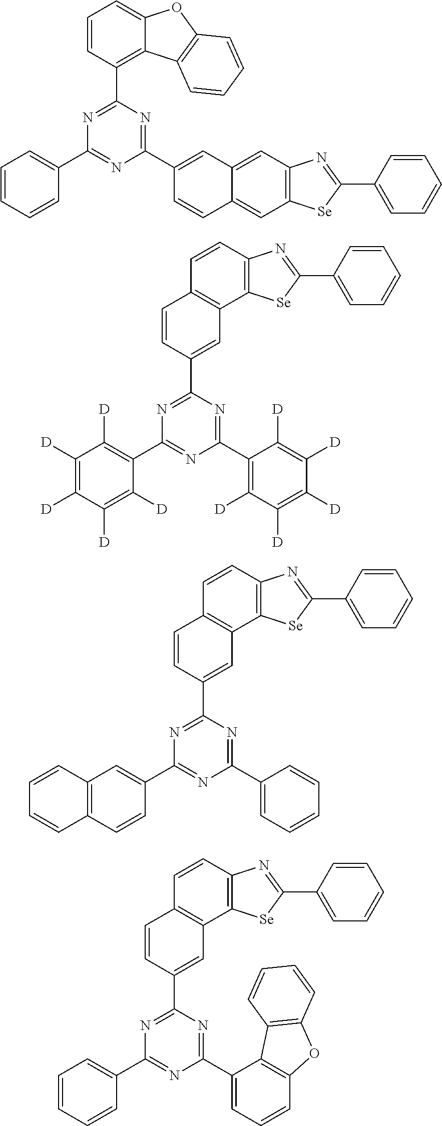

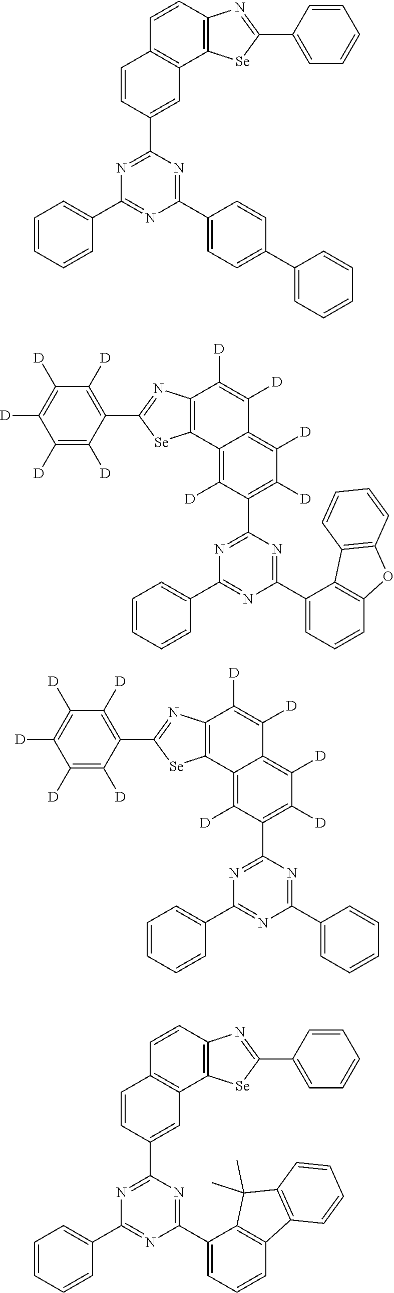 [SMILES: c1ccc(-c2ccc(-c3nc(-c4ccccc4)nc(-c4ccc5ccc6nc(-c7ccccc7)[se]c6c5c4)n3)cc2)cc1 ; [2H]c1c([2H])c([2H])c(-c2nc3c([2H])c([2H])c4c([2H])c([2H])c(-c5nc(-c6ccccc6)nc(-c6cccc7oc8ccccc8c67)n5)c([2H])c4c3[se]2)c([2H])c1[2H] ; [2H]c1c([2H])c([2H])c(-c2nc3c([2H])c([2H])c4c([2H])c([2H])c(-c5nc(-c6ccccc6)nc(-c6ccccc6)n5)c([2H])c4c3[se]2)c([2H])c1[2H] ; CC1(C)c2ccccc2-c2cccc(-c3nc(-c4ccccc4)nc(-c4ccc5ccc6nc(-c7ccccc7)[se]c6c5c4)n3)c21]

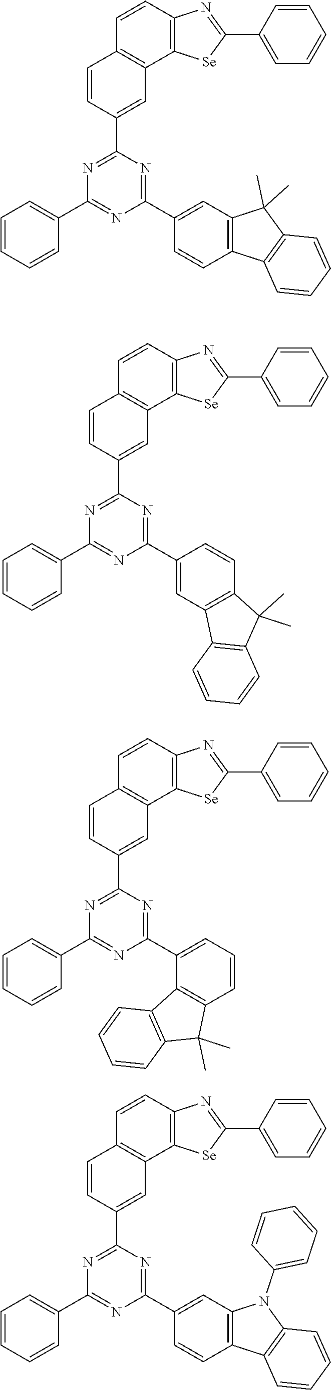

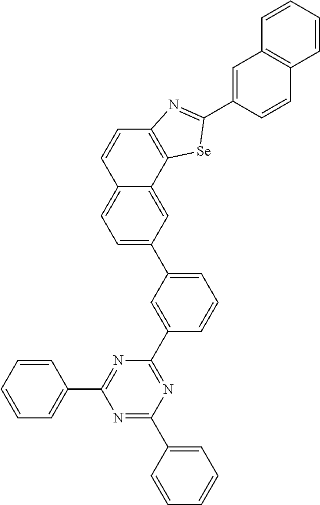 [SMILES: c1ccc(-c2nc(-c3ccccc3)nc(-c3cccc(-c4ccc5ccc6nc(-c7ccc8ccccc8c7)[se]c6c5c4)c3)n2)cc1]

The dopants comprised in the organic electroluminescent device of the present disclosure may be at least one phosphorescent or fluorescent dopant, and is preferably a phosphorescent dopant. The phosphorescent dopant materials applied to the organic electroluminescent device according to the present disclosure are not particularly limited, but may be a complex compound of a metal atom selected from iridium (Ir), osmium (Os), copper (Cu), and platinum (Pt), preferably ortho-metallated complex compounds of a metal atom selected from iridium (Ir), osmium (Os), copper (Cu), and platinum (Pt), and more preferably ortho-metallated iridium complex compounds.

The dopant comprised in the organic electroluminescent device of the present disclosure may be a compound represented by the following formula 101, but is not limited thereto.

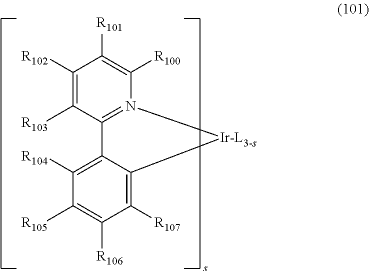

In formula 101,
L is selected from the following structures 1 to 3:

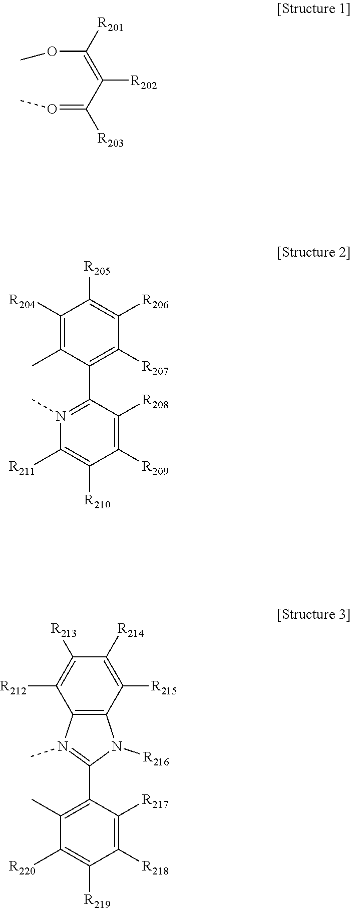

R₁₀₀ to R₁₀₃ each independently represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent(s) to form a ring(s), for example, a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline, together with pyridine;
R₁₀₄ to R₁₀₇ each independently represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent(s) to form a ring(s), for example, a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine, together with benzene;
R₂₀₁ to R₂₂₀ each independently represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent(s) to form a substituted or unsubstituted ring(s); and
s is an integer from 1 to 3.

The specific examples of the dopant compound are as follows, but are not limited thereto.

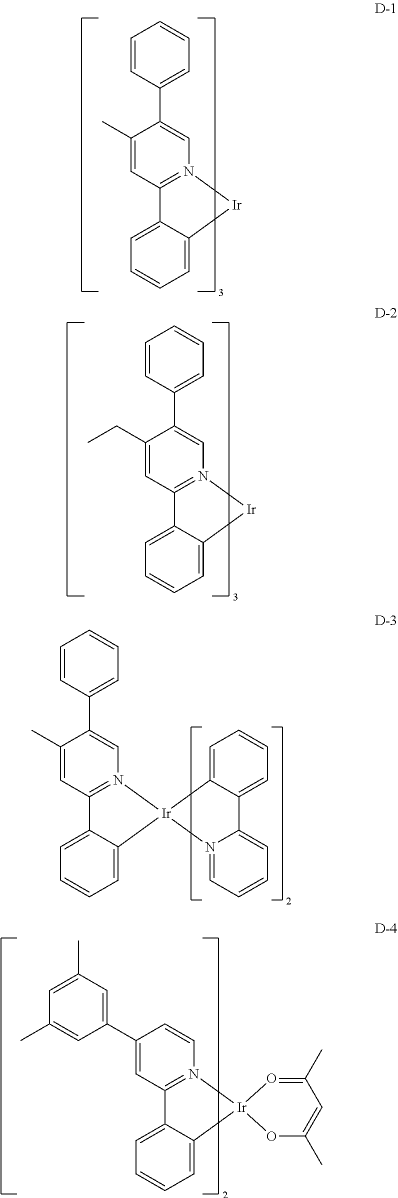

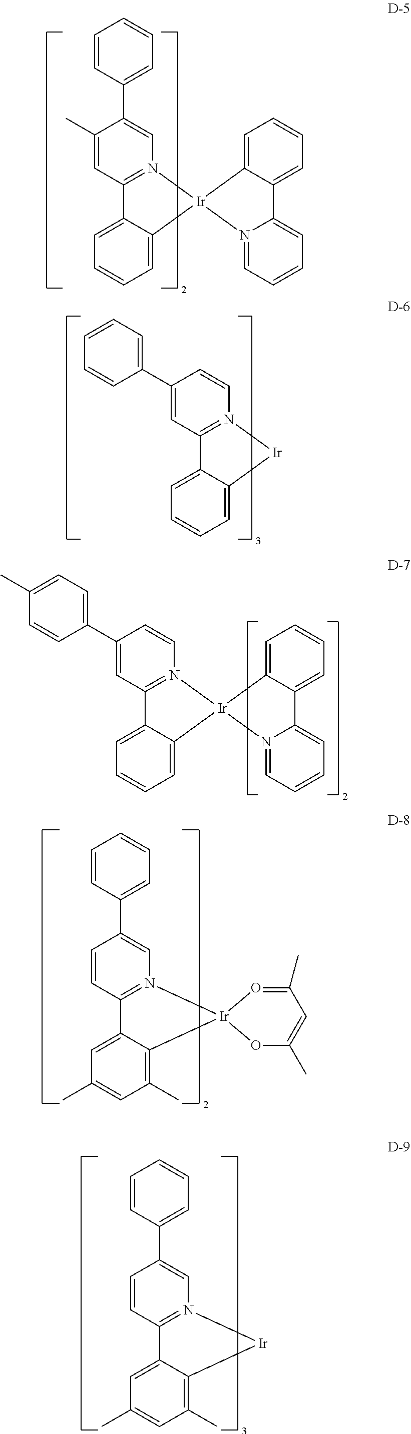

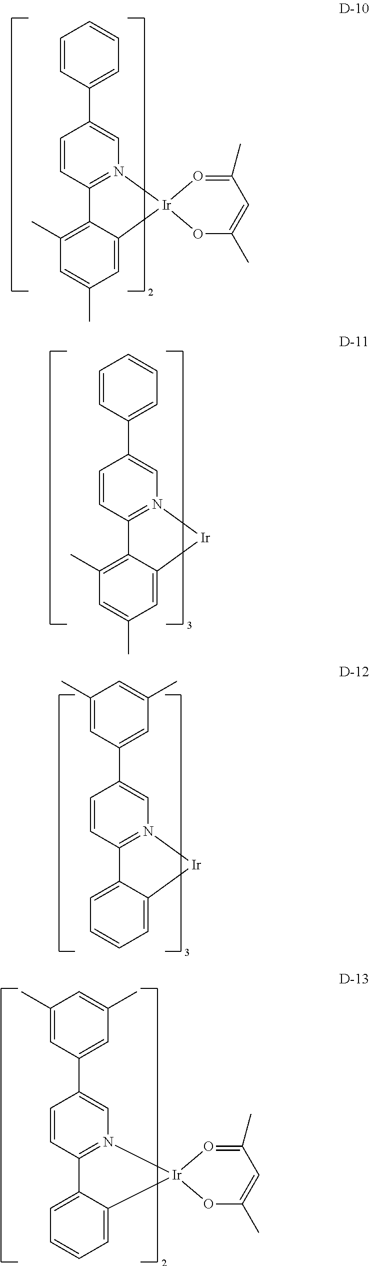

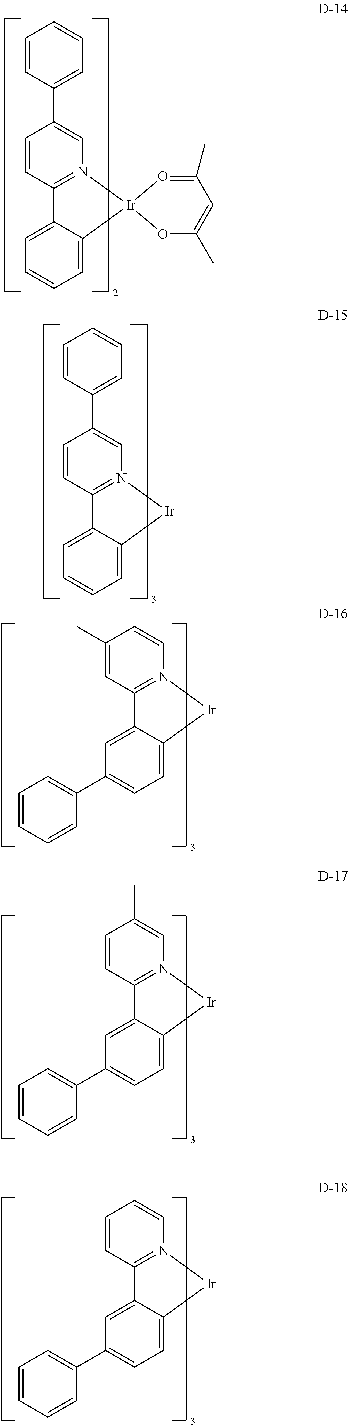

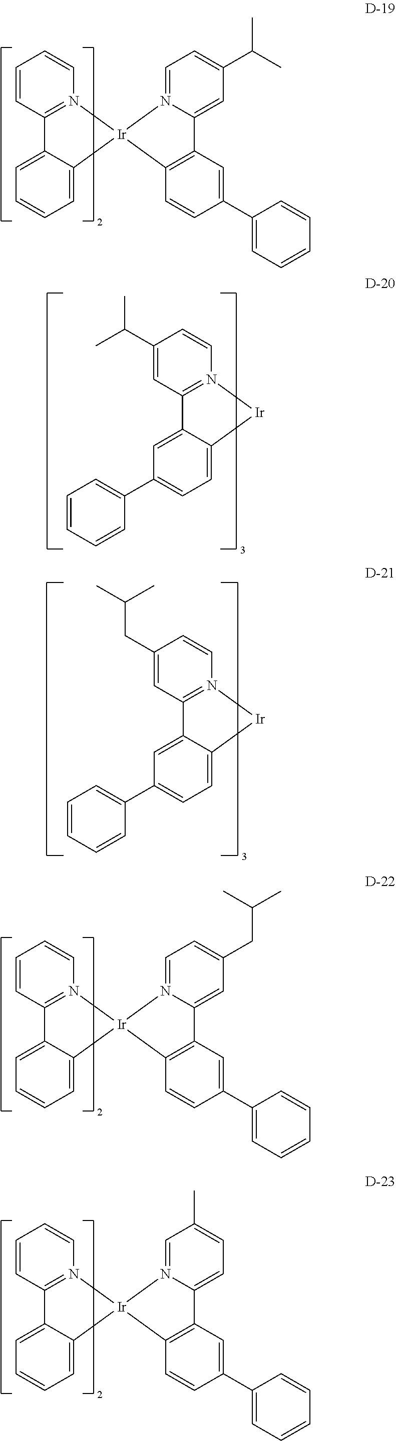

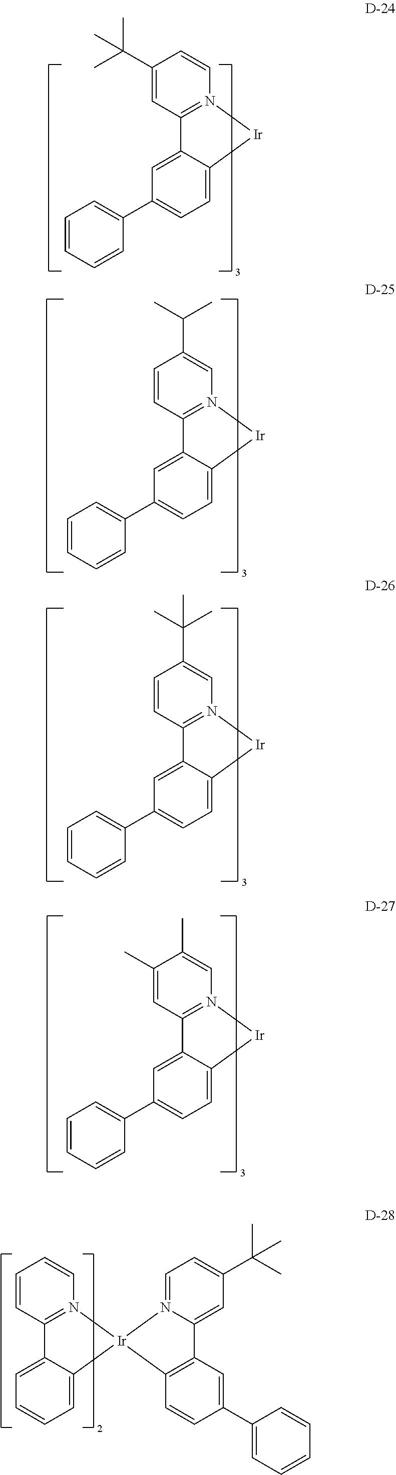

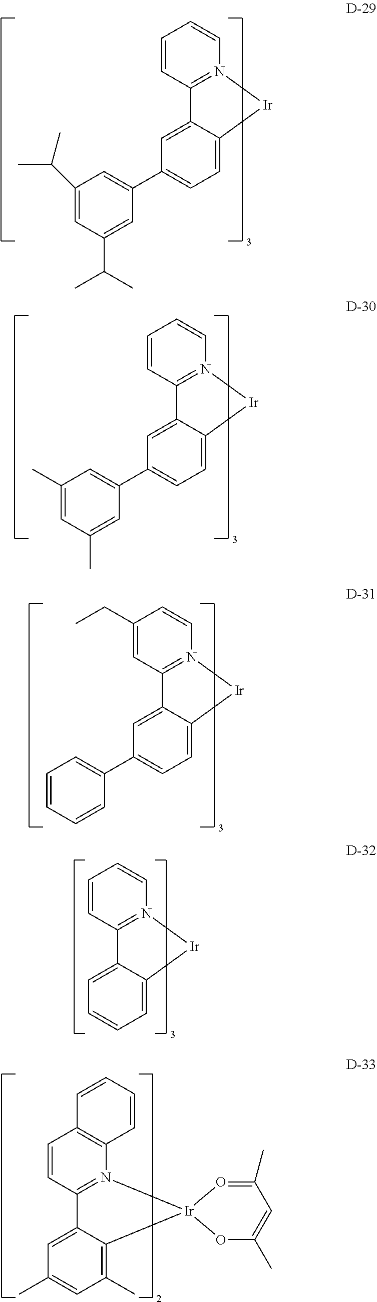

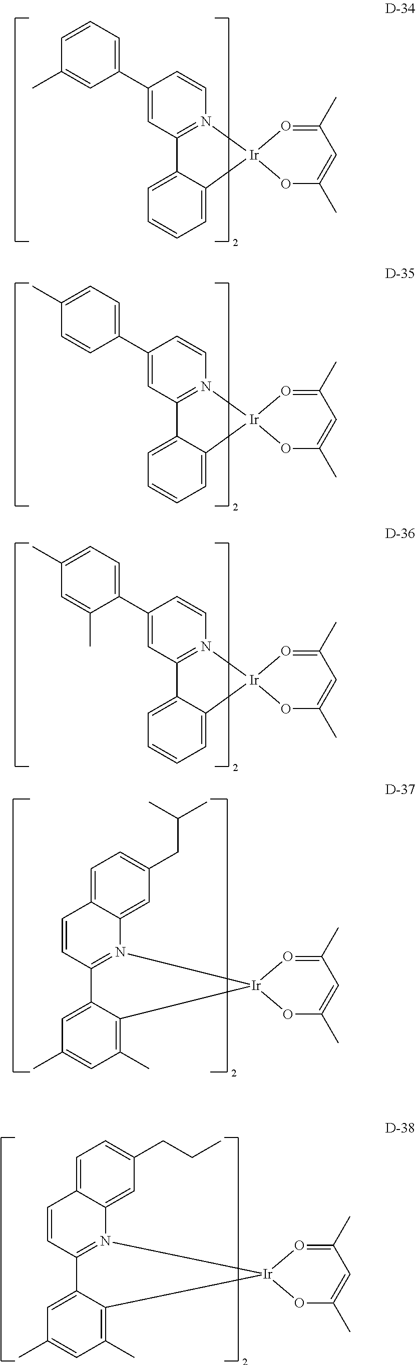

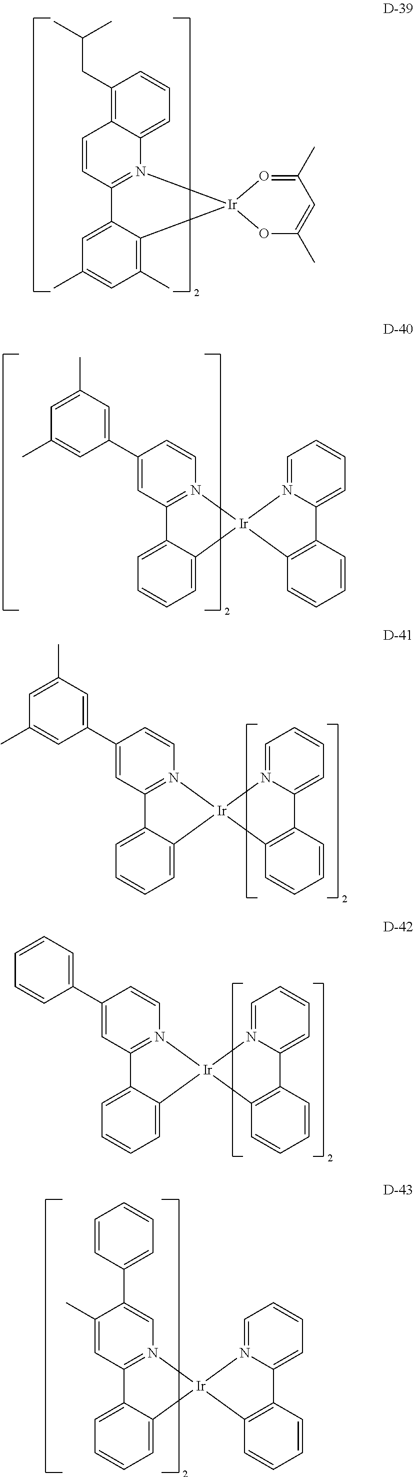

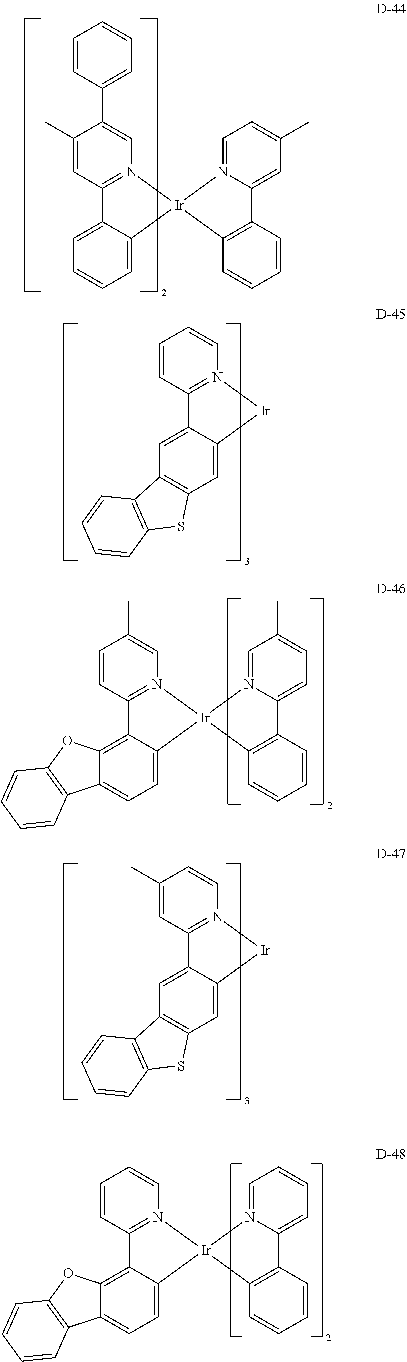

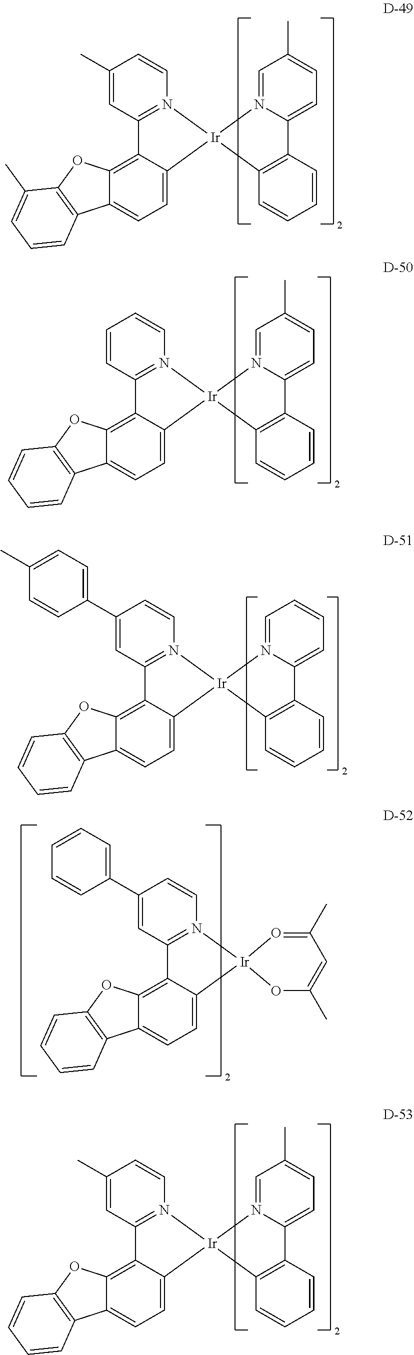

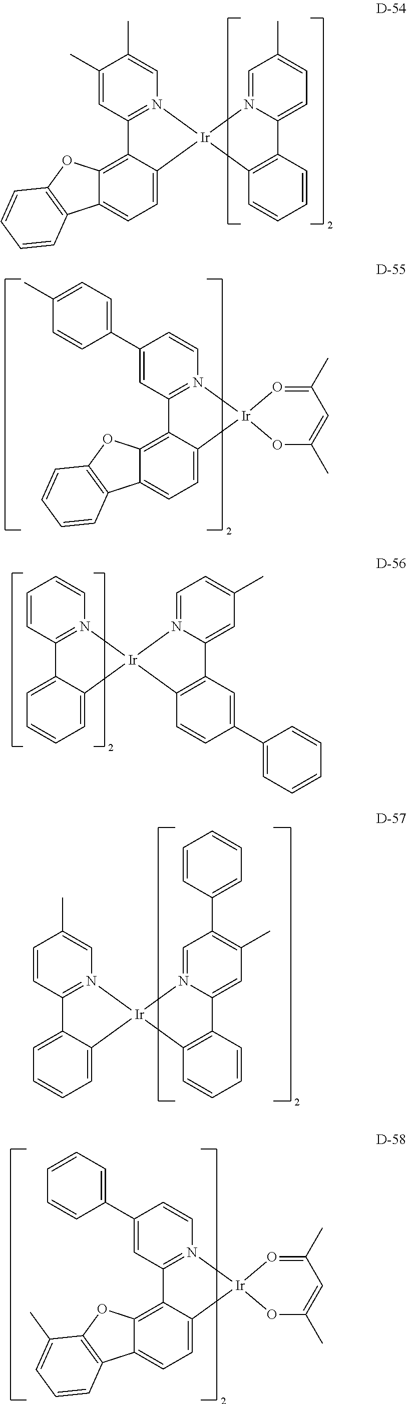

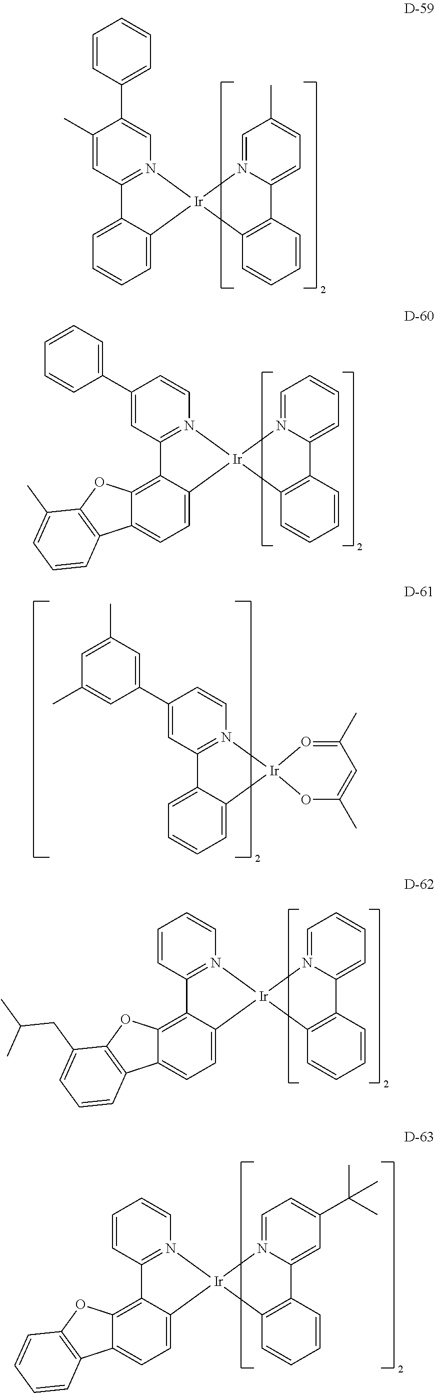

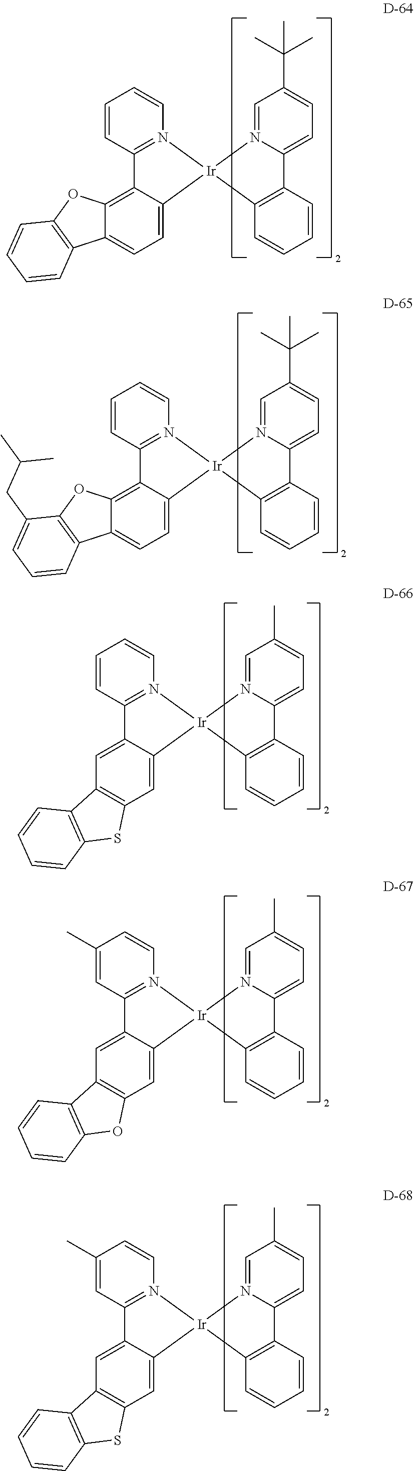

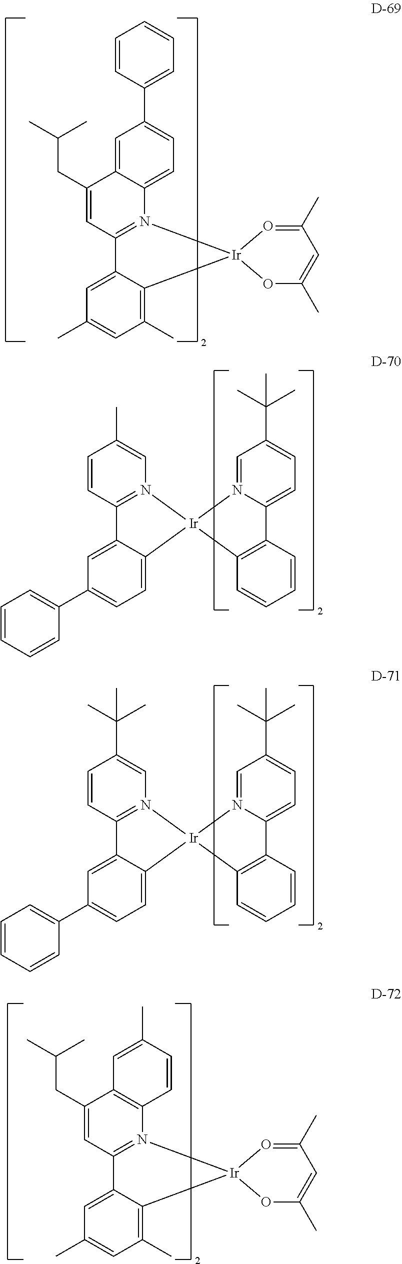

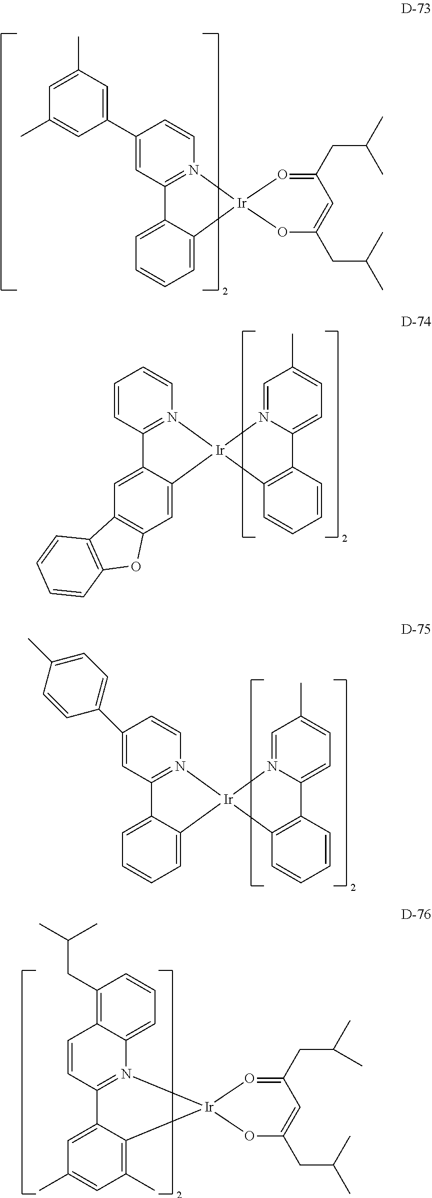

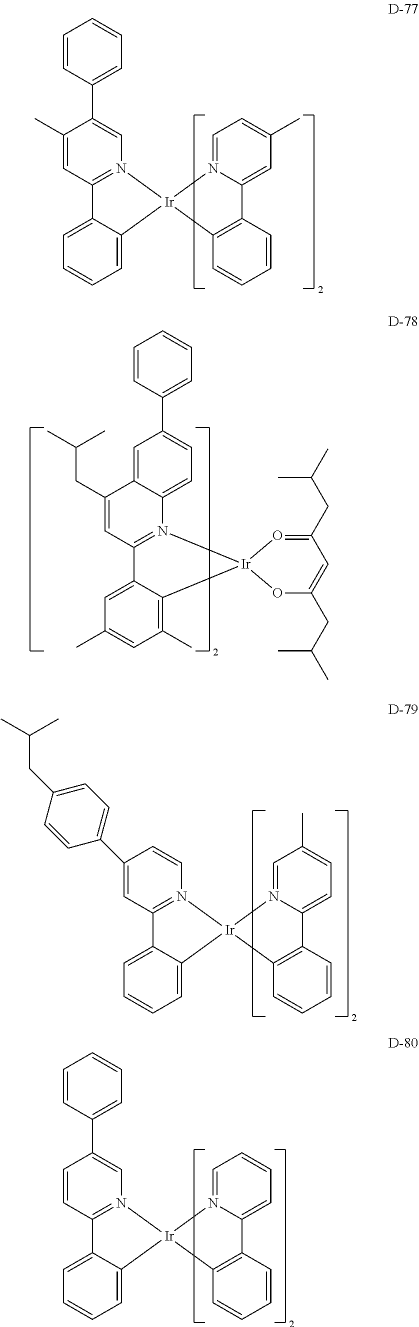

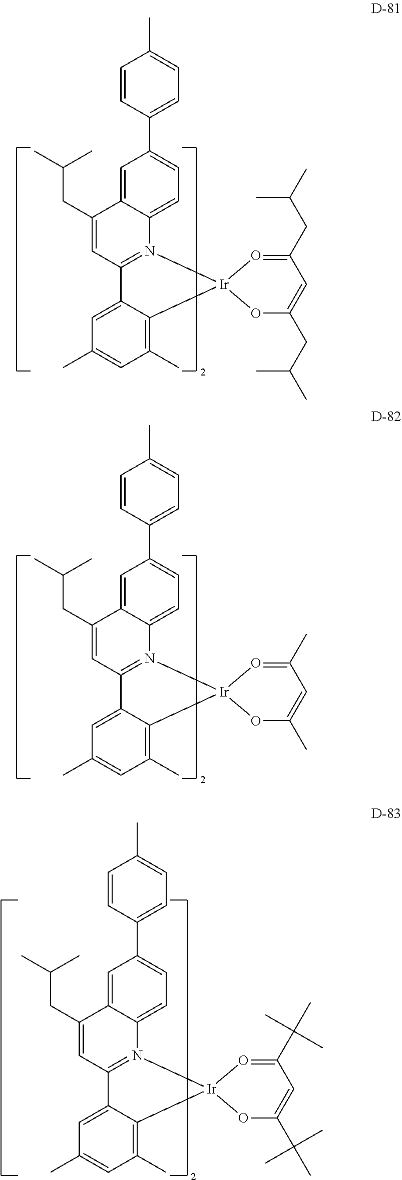

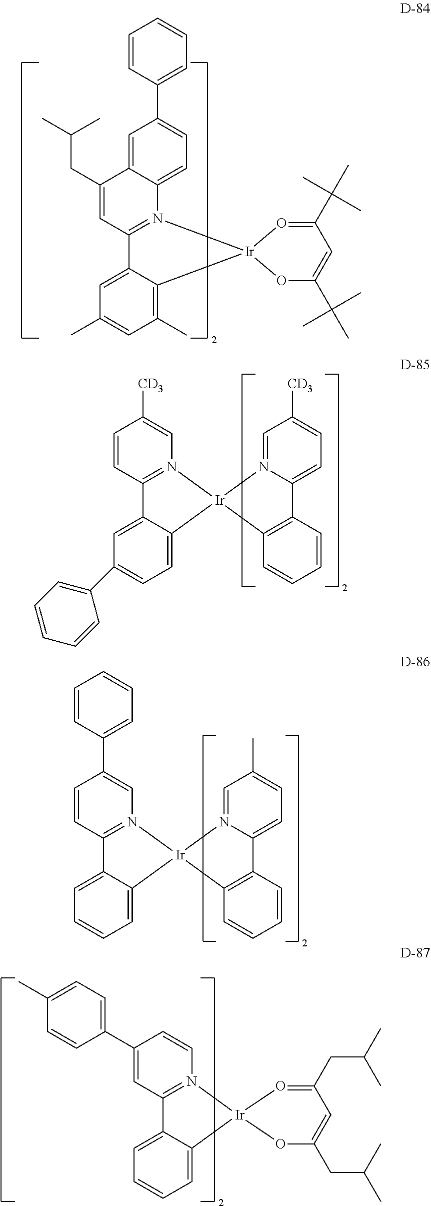

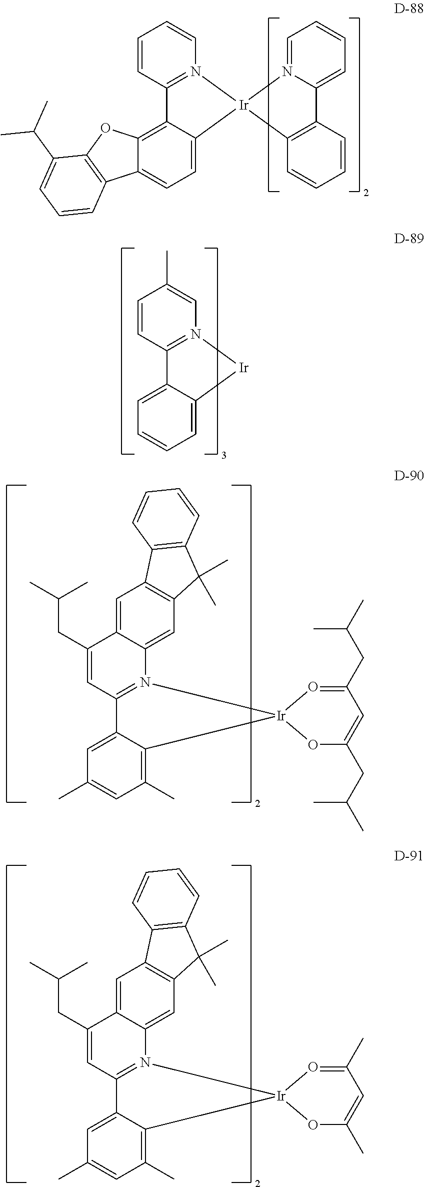

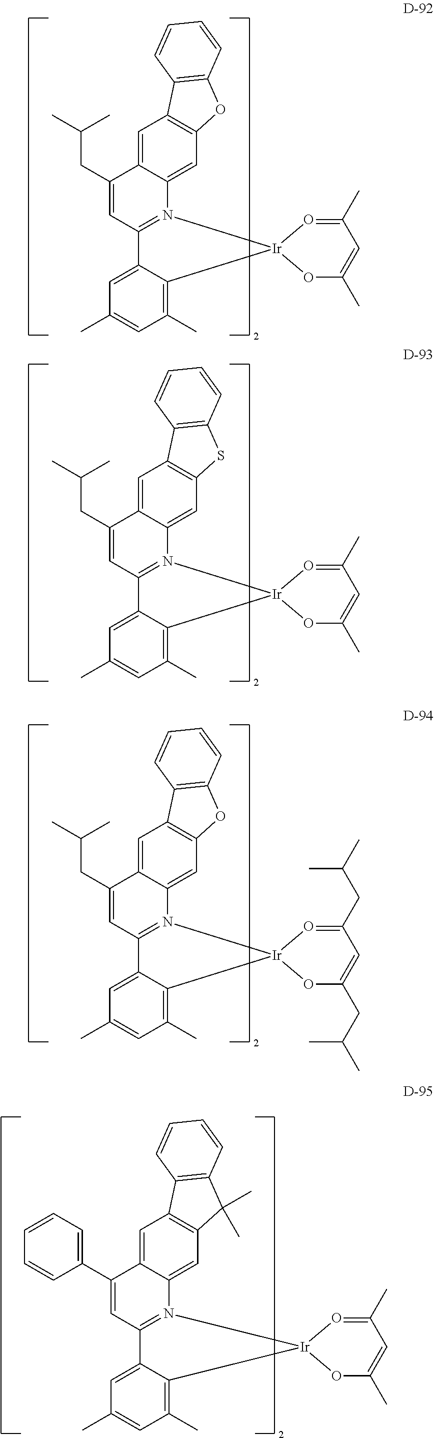

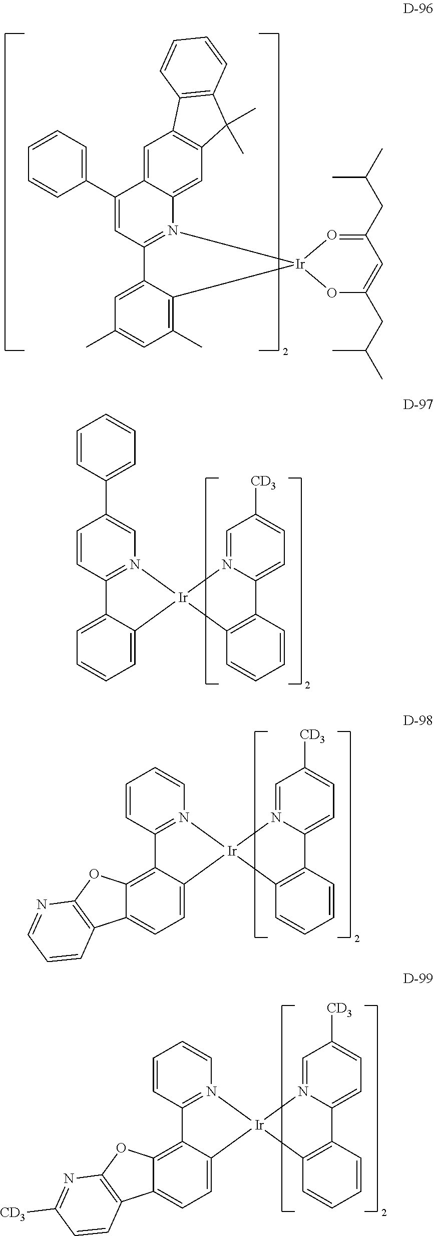

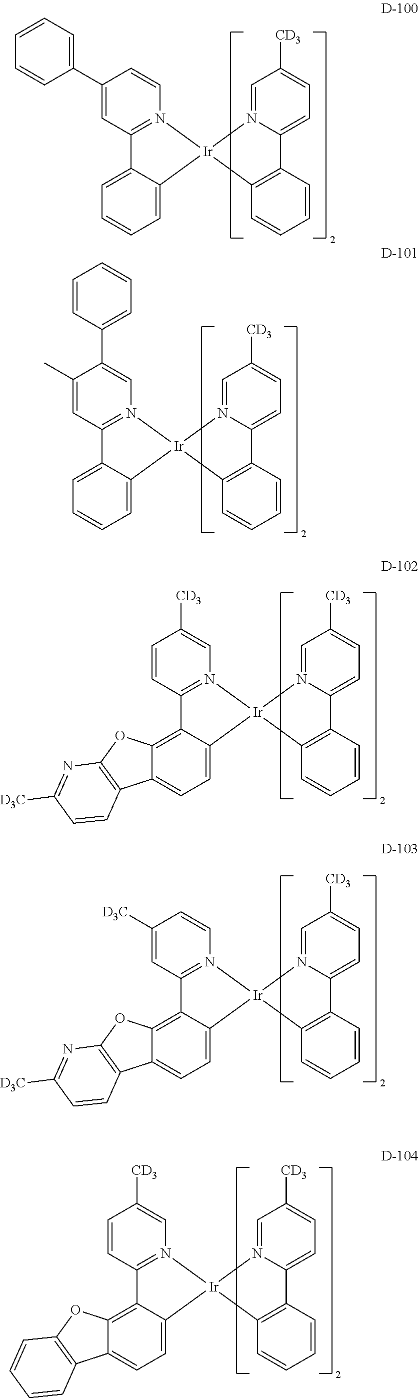

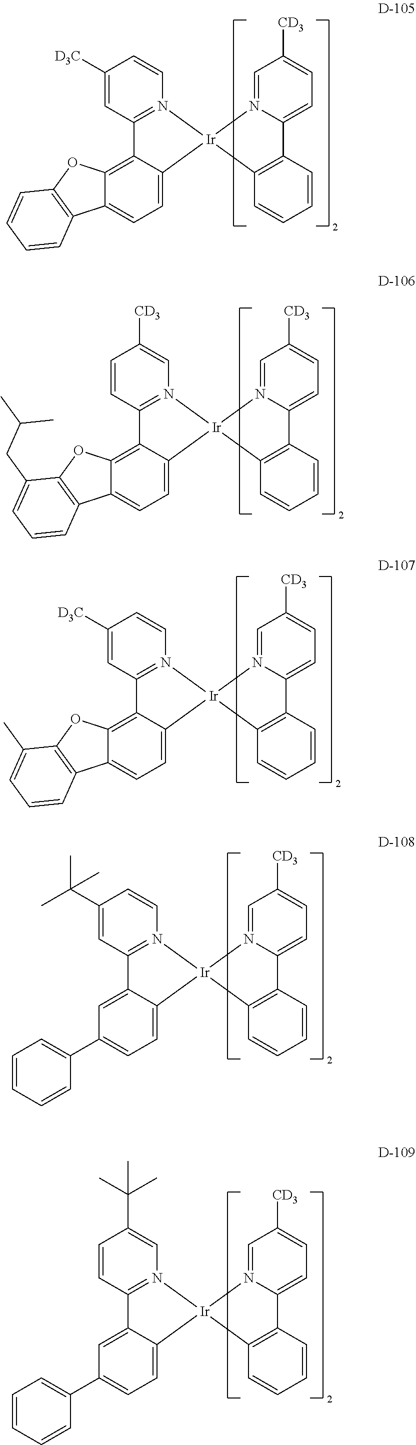

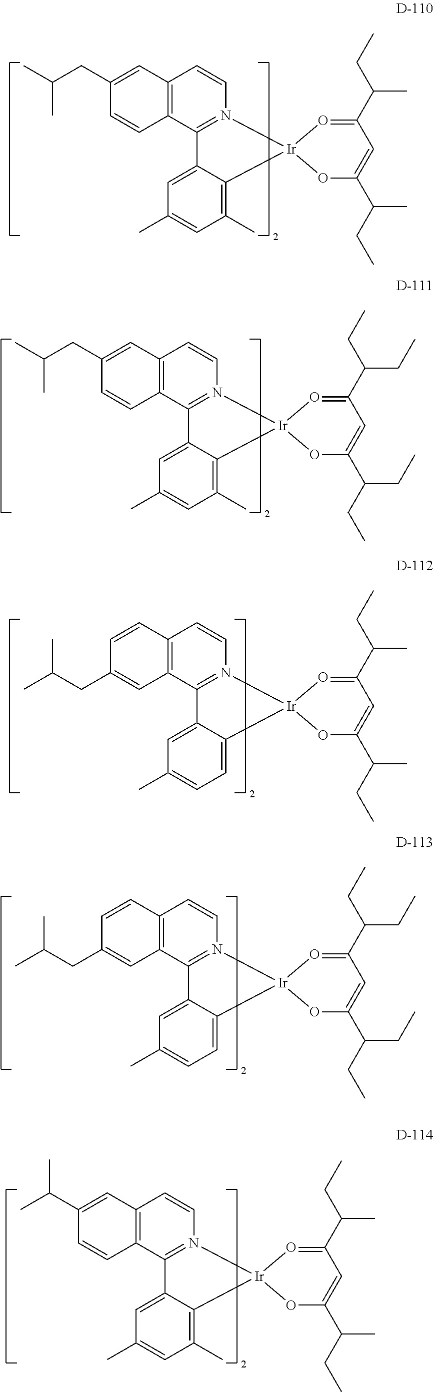

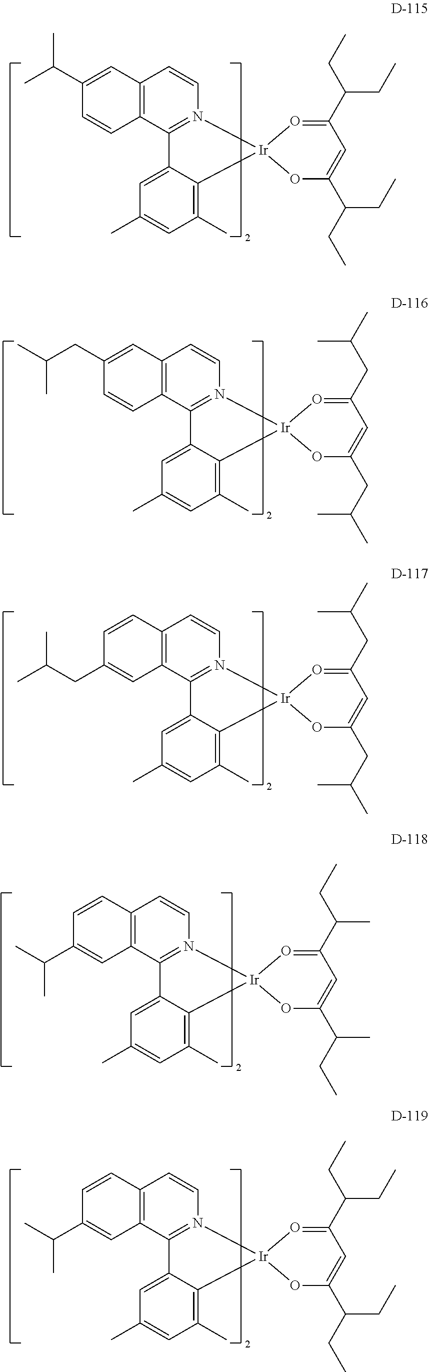

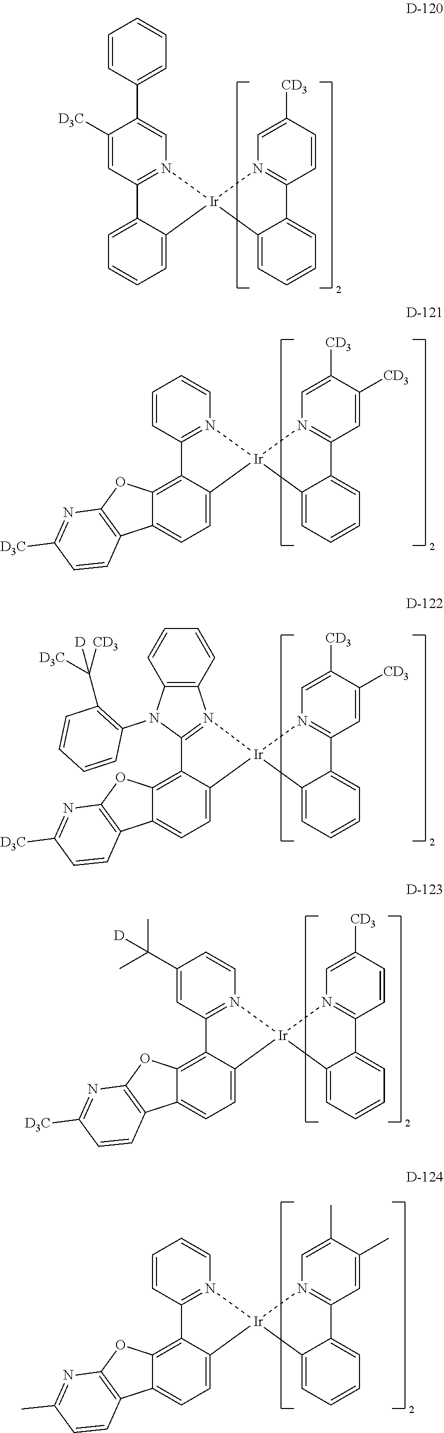

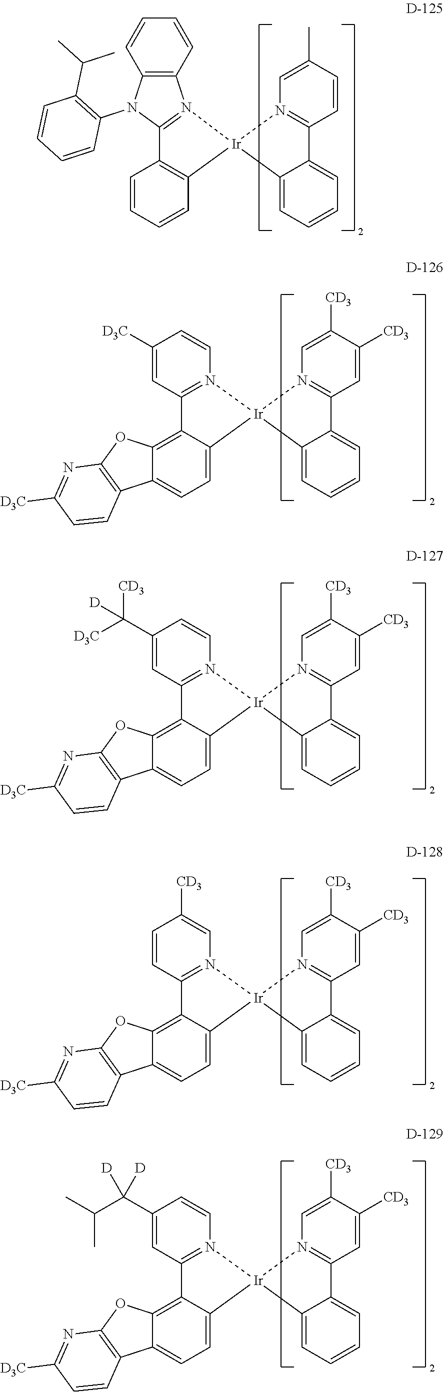

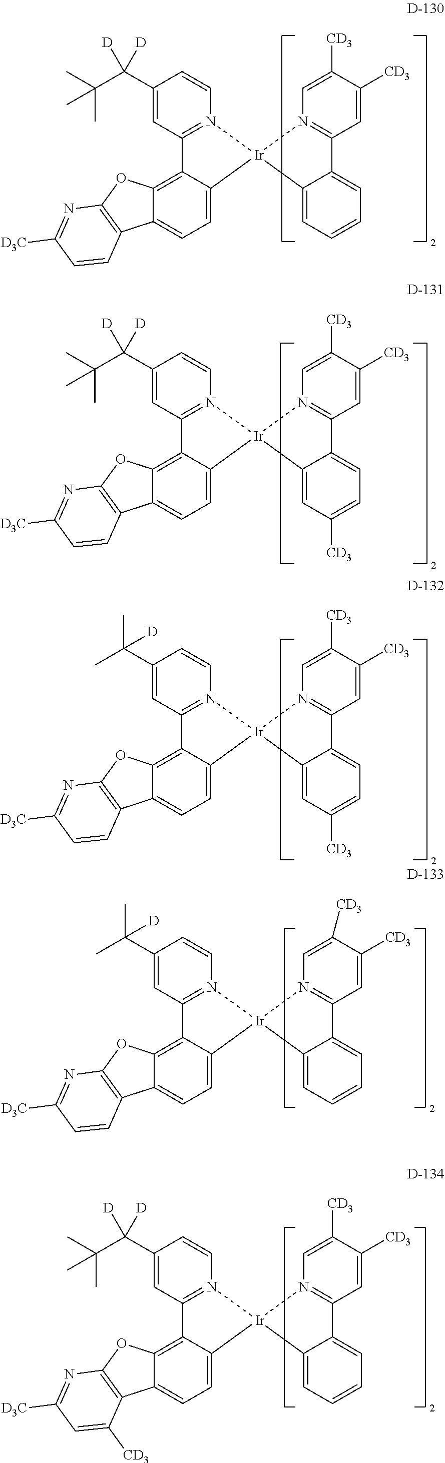

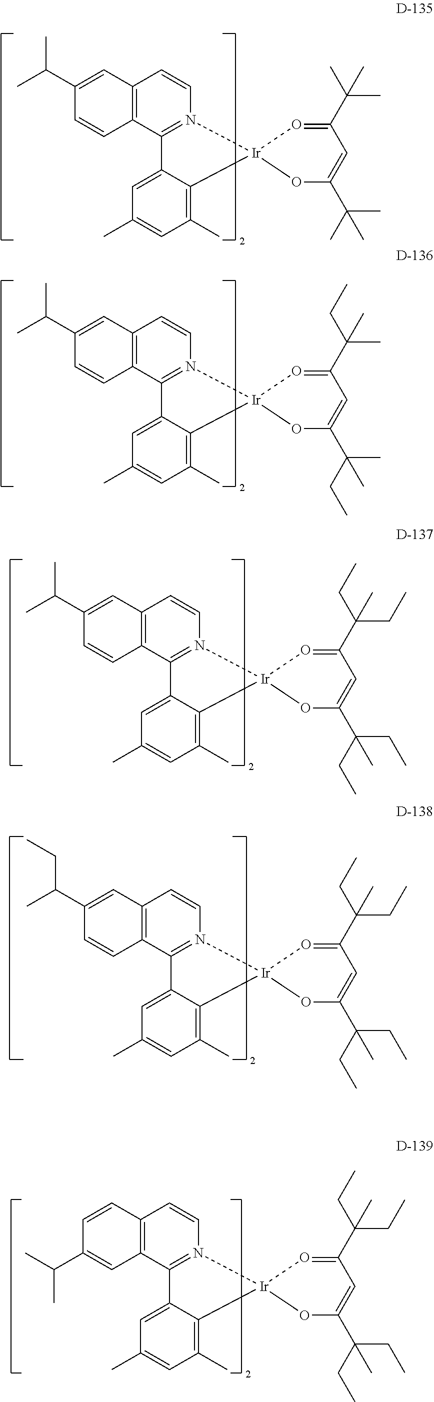

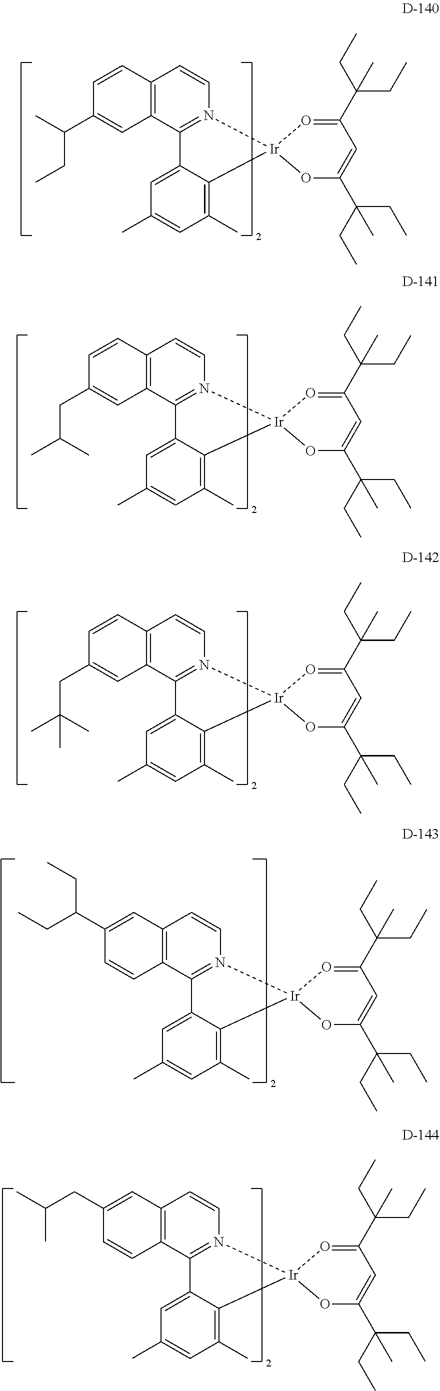

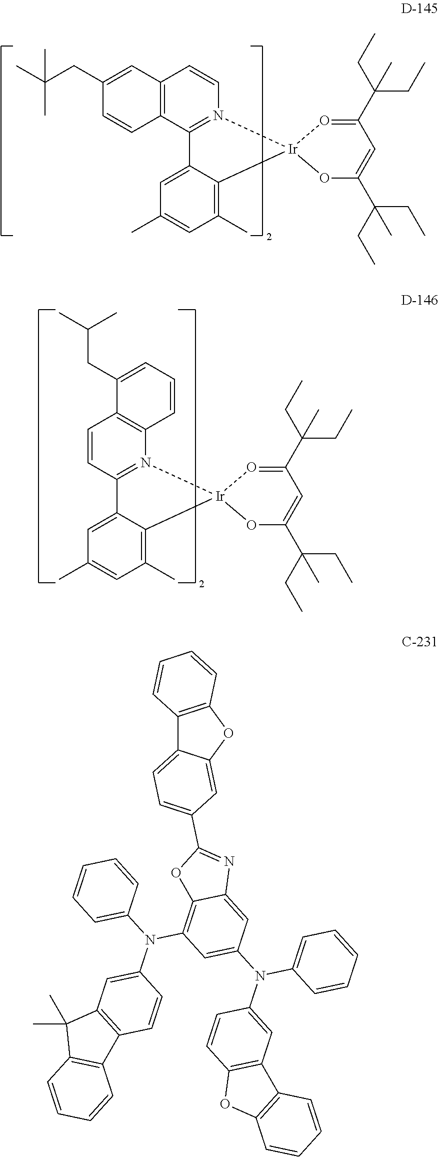

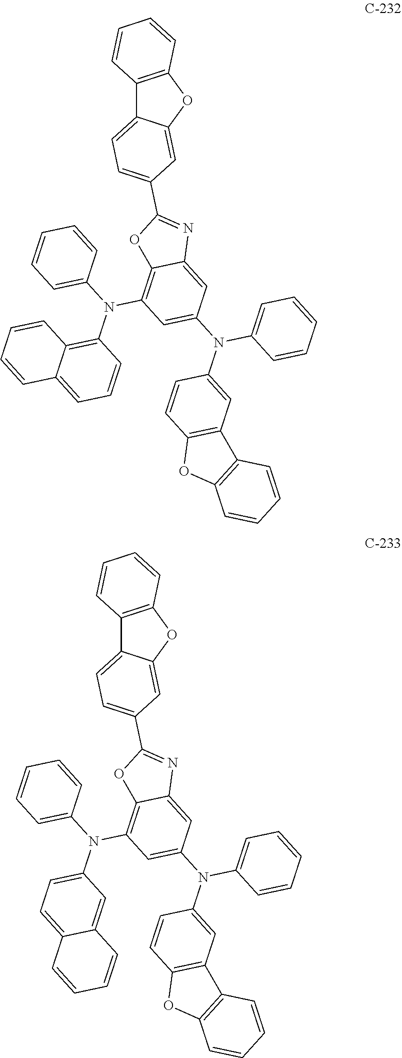 [SMILES: c1ccc(N(c2cc(N(c3ccccc3)c3cccc4ccccc34)c3oc(-c4ccc5c(c4)oc4ccccc45)nc3c2)c2ccc3oc4ccccc4c3c2)cc1 ; c1ccc(N(c2cc(N(c3ccccc3)c3ccc4ccccc4c3)c3oc(-c4ccc5c(c4)oc4ccccc45)nc3c2)c2ccc3oc4ccccc4c3c2)cc1]

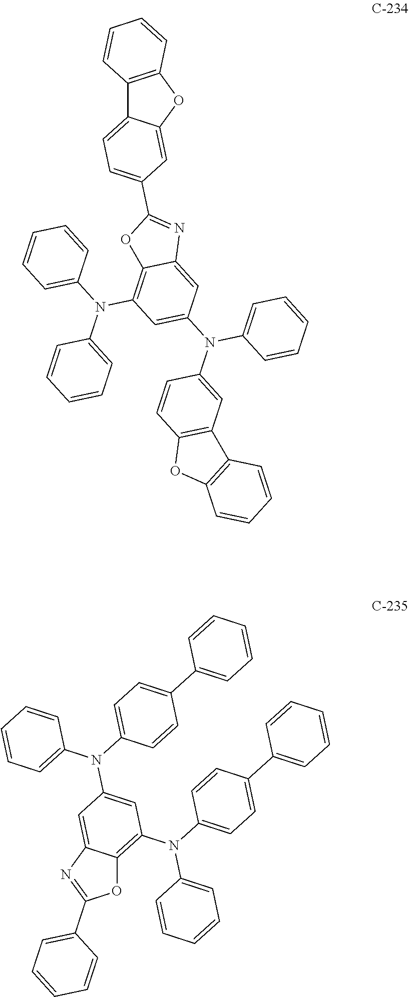 [SMILES: c1ccc(N(c2cc(N(c3ccccc3)c3ccccc3)c3oc(-c4ccc5c(c4)oc4ccccc45)nc3c2)c2ccc3oc4ccccc4c3c2)cc1 ; c1ccc(-c2ccc(N(c3ccccc3)c3cc(N(c4ccccc4)c4ccc(-c5ccccc5)cc4)c4oc(-c5ccccc5)nc4c3)cc2)cc1]

The compound represented by formula 1 may be comprised in at least one layer constituting an organic electroluminescent device, for example, at least one layer selected from a hole injection layer, a hole transport layer, a hole auxiliary layer, a light-emitting auxiliary layer, a light-emitting layer, an electron transport layer, an electron buffer layer, an electron injection layer, an interlayer, a hole blocking layer, and an electron blocking layer. Each of the layers may be further configured as a plurality of layers.

In addition, the compound represented by formula 1 may be comprised in a hole transport zone and/or a light-emitting layer, but is not limited thereto. The compound represented by formula 1 may be comprised in the hole transport zone as at least one of a hole injection material, a hole transport material, a hole auxiliary material, a light-emitting auxiliary material, and an electron blocking material, for example as a hole transport material. Further, the compound represented by formula 1 may be comprised in a light-emitting layer as a host, and may be used as a host having a hole transport property among hosts.

An organic electroluminescent device of the present disclosure may comprise a hole transport zone between an anode and a light-emitting layer. The transport zone may comprise at least one of a hole injection layer, a hole transport layer, a hole auxiliary layer, a light-emitting auxiliary layer, and an electron blocking layer. The hole injection layer, the hole transport layer, the hole auxiliary layer, the light-emitting auxiliary layer, and the electron blocking layer may be respectively a single layer, or multi-layers in which two or more layers are stacked. The hole injection layer may be multi-layers in order to lower the hole injection barrier (or hole injection voltage) from the anode to the hole transport layer or the electron blocking layer, wherein each of the multi-layers may use two compounds simultaneously. The electron blocking layer may be placed between the hole transport layer (or hole injection layer) and the light-emitting layer, and can confine the excitons within the light-emitting layer by blocking the overflow of electrons from the light-emitting layer to prevent a light-emitting leakage.

In addition, the hole transport zone may comprise a p-doped hole injection layer, a hole transport layer, and a light-emitting auxiliary layer. Herein, the p-doped hole injection layer means a hole injection layer doped with a p-dopant. The p-dopant is a material that imparts a p-semiconductor property. The p-semiconductor property means a property that injects or transports holes to a HOMO energy level, i.e., a property of a material having high hole conductivity.

At least one of organic electroluminescent materials of the present disclosure, for example, a hole injection material, a hole transport material, a hole auxiliary material, a light-emitting auxiliary material, an electron blocking material, a light-emitting material, an electron buffer material, a hole blocking material, an electron transport material, an electron injection material, and a light-emitting material (host material) may comprise the compound represented by formula 1. The organic electroluminescent material may be a hole transport material and/or a light-emitting material. The organic electroluminescent material may consist of the compound represented by formula 1 alone, or may further comprise conventional materials comprised in an organic electroluminescent material. For example, the organic electroluminescent material may further comprise at least one compound represented by any one of formulas 11 to 15. When two or more materials are comprised in one layer, they may be mixture-evaporated to form a layer or they may be separately co-evaporated at the same time to form a layer.

An organic electroluminescent device of the present disclosure comprises a first electrode; a second electrode; and at least one organic layer between the first electrode and the second electrode. One of the first electrode and the second electrode may be an anode, and the other may be a cathode. The organic layer comprises at least one light-emitting layer, and may further comprise at least one layer of a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer.

Each of the first electrode and the second electrode may be formed of a transparent conductive material, or a transflective or reflective conductive material. The organic electroluminescent device may be a top emission type, a bottom emission type, or a both-sides emission type depending on the types of materials forming the first electrode and the second electrode. In addition, the hole injection layer may further be doped with a p-dopant, and the electron injection layer may further be doped with an n-dopant.

An organic electroluminescent device of the present disclosure may comprise the compound represented by formula 1 and may further comprise conventional materials comprised in an organic electroluminescent material. An organic electroluminescent device comprising the organic electroluminescent compound represented by formula 1 may exhibit low driving voltage and/or high power efficiency properties.

In addition, an organic electroluminescent material according to one embodiment may be used as light-emitting materials for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or color conversion material (CCM) method, etc. The present disclosure may also be applied to such a white organic light-emittring device. In addition, the organic electroluminescent material according to one embodiment may also be applied to the organic electroluminescent device comprising a quantum dot (QD).

Further, the present disclosure may provide a display system by using the compound represented by formula 1. That is, it is possible to manufacture a display system or a light system by using the compound of the present disclosure. Specifically, it is possible to manufacture a display system, for example, a display system for smart phones, tablets, notebooks, PCs, TVs, or cars; or a lighting system, for example, an outdoor or indoor lighting system by using the compound of the present disclosure.

Hereinafter, the preparation method of the compound according to the present disclosure and the physical properties thereof, and the characteristics of the organic electroluminescent device (OLED) comprising the organic electroluminescent compound of the present disclosure will be explained with reference to the representative compounds of the present disclosure. However, the following examples only describe the characteristics of the compound and the OLED device comprising the same according to the present disclosure, but the present disclosure is not limited to the following examples.

### Example 1: Preparation of Compound C-1

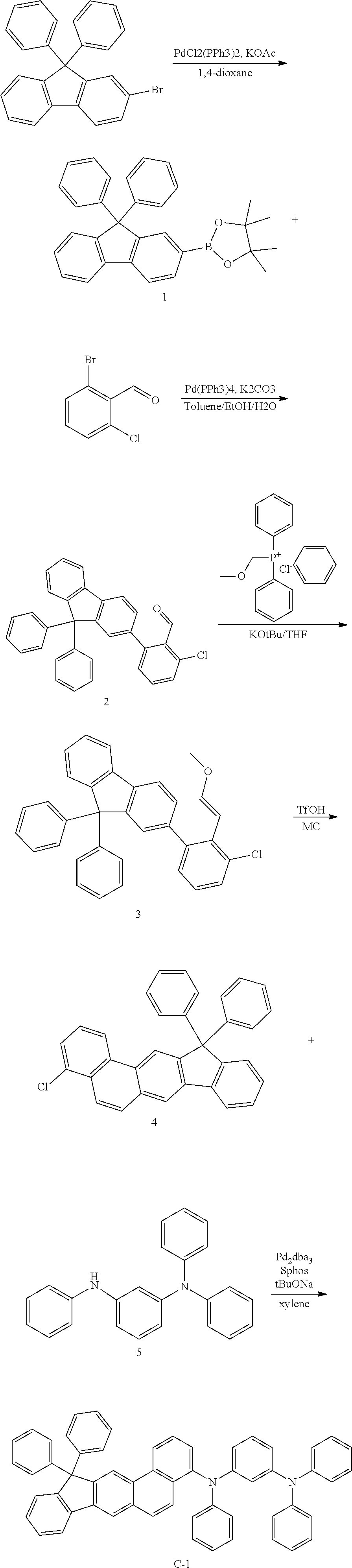

Synthesis of Compound 1

In a flask, 2-bromo-9,9-diphenyl-9H-fluorene (30 g, 75.5 mmol), (4,4,4′,4′,5,5,5′,5′-octamethyl-2,2′-bi(1,3,2-dioxaborolan) (28 g, 113.2 mmol), KOAc (14.8 g, 151 mmol), and PdCl₂(PPh₃)₂ (4.3 g, 3.77 mmol) were dissolved in 500 mL of 1,4-dioxane, and stirred under reflux at 150° C. for 3 hours. After completion of the reaction, the organic layer was extracted with ethyl acetate, and the residual moisture was removed using magnesium sulfate. Thereafter, the residue was dried and separated by column chromatography to obtain Compound 1 (35 g, yield: 100%).

Synthesis of Compound 2

Compound 1 (35 g, 78.7 mmol), 2-bromo-6-chlorobenzaldehyde (17 g, 78.7 mmol), Pd(PPh₃)₄ (4.5 g, 3.93 mmol), K2CO3 (21 g, 154.8 mmol), 546 mL of toluene, 273 mL of EtOH, and 273 mL of H₂O were added to a flask, and stirred under reflux at 130° C. for 3 hours. After completion of the reaction, methanol and water were added and stirred, and then the solvent was removed by filtration under reduced pressure. Thereafter, the residue was separated by column chromatography, and methanol was added to produce a solid. The resulting solid was filtered under reduced pressure to obtain Compound 2 (30 g, yield: 83.4%).

Synthesis of Compound 3

In a flask, Compound 2 (30 g, 65.6 mmol), (methoxymethyl)triphenylphosphoniumchloride (33 g, 98.5 mmol), and KOtBu (15.1 g, 98.5 mmol) were dissolved in 326 mL of THF. The mixture was stirred at room temperature for 2 hours. After completion of the reaction, MeOH and water were added and stirred. Next, the solvent was removed by filtration under reduced pressure, and the residue was separated by column chromatography. After adding MeOH, the resulting solid was filtered under reduced pressure to obtain Compound 3 (40 g, yield: over yield %).

Synthesis of Compound 4

In a flask, Compound 3 (40 g, 82.4 mmol) was dissolved in 500 mL of methylene chloride, and TfOH (trifluoromethanesulfonic acid) (7.2 mL, 82.4 mmol) was slowly added dropwise. The mixture was stirred at room temperature for 1 hour. After completion of the reaction, MeOH and water were added and stirred.

Next, the solvent was removed by filtration under reduced pressure, and the residue was separated by column chromatography. After adding MeOH, the resulting solid was filtered under reduced pressure to obtain Compound 4 (24 g, yield: 64%).

Synthesis of Compound C-1

In a flask, Compound 4 (5 g, 11 mmol), Compound 5 (5.9 g, 12.1 mmol), NaOtBu (2.1 g, 22 mmol), S-Phos (361 mg, 0.88 mmol), and Pd₂(dba)₃ (503 mg, 0.55 mmol) were dissolved in 75 mL of xylene, and stirred under reflux at 130° C. for 12 hours. After completion of the reaction, the organic layer was extracted with ethyl acetate, and the residual moisture was removed using magnesium sulfate. Thereafter, the residue was dried and separated by column chromatography to obtain Compound C-1 (3.5 g, yield: 37.1%).

| | MW | M.P. (° C.) |
|---|---|---|
| C-1 | 752.96 | 193.8 |

### Example 2: Preparation of Compound C-2

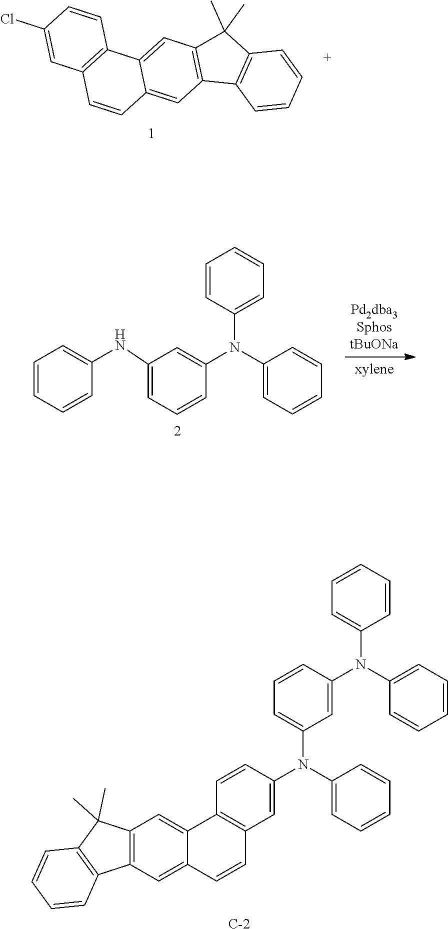

In a flask, Compound 1 (3 g, 9.1 mmol), Compound 2 (4.9 g, 10 mmol), NaOtBu (2.2 g, 22.75 mmol), S-Phos (298 mg, 0.728 mmol), and Pd₂(dba)₃ (416 mg, 0.455 mmol) were dissolved in 46 mL of xylene, and stirred under reduced pressure at 130° C. for 2 hours. After completion of the reaction, the organic layer was extracted with ethyl acetate, and the residual moisture was removed using magnesium sulfate. Thereafter, the residue was dried and separated by column chromatography to obtain Compound C-2 (3.1 g, yield: 54.2%).

| | MW | M.P. (° C.) |
|---|---|---|
| C-2 | 628.82 | 214.7 |

Device Examples 1 and 2: Manufacture of OLEDs Co-Deposited with the First Host Compound and the Second Host Compound According to the Present Disclosure

An OLED according to the present disclosure was manufactured. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was mounted on a substrate holder of a vacuum vapor deposition apparatus. Compound HI-1 was introduced into a cell of the vacuum vapor deposition apparatus, and compound HT-1 was introduced into another cell. The two materials were evaporated at different rates, and compound HI-1 was deposited in a doping amount of 3 wt % based to the total amount of compound HI-1 and compound HT-1 to form a hole injection layer with a thickness of 10 nm. Subsequently, compound HT-1 was deposited on the hole injection layer to form a first hole transport layer with a thickness of 80 nm. Next, compound HT-2 was introduced into another cell of the vacuum vapor deposition apparatus and was evaporated by applying an electric current to the cell, thereby depositing a second hole transport layer with a thickness of 60 nm on the first hole transport layer. After forming the hole injection layer and the hole transport layers, a light-emitting layer was deposited thereon as follows: the first host compound and the second host compound shown in Table 1 below were respectively introduced into two cells of the vacuum vapor deposition apparatus as hosts, and compound D-39 was introduced into another cell as a dopant. The two host compounds were evaporated at a rate of 1:1 and the dopant material was simultaneously evaporated at a different rate, and the dopant was deposited in a doping amount of 3 wt % based on the total amount of the hosts and dopant to form a light-emitting layer with a thickness of 40 nm on the second hole transport layer. Then, compound ETL-1 and compound EIL-1 were evaporated at a weight ratio of 50:50 as an electron transport material to form an electron transport layer having a thickness of 35 nm on the light-emitting layer. After depositing compound EIL-1 as an electron injection layer with a thickness of 2 nm on the electron transport layer, an Al cathode was deposited with a thickness of 80 nm on the electron injection layer by using another vacuum vapor deposition apparatus, thereby producing an OLED. All the materials used for manufacturing the OLED were purified by vacuum sublimation at 10⁻⁶ torr.

### Comparative Example 1: Manufacture of an OLED Comprising a Single Host

An OLED was manufactured in the same manner as in Device Example 1, except that Compound RH-1 was used alone as a host of the light-emitting layer.

### Comparative Example 2: Manufacture of an OLED Comprising a Comparative Compound as a Host

An OLED was manufactured in the same manner as in Device Example 1, except that the first host compound shown in Table 1 below was used as a host of the light-emitting layer.

The driving voltage, the luminous efficiency, and the light-emitting color at a luminance of 1,000 nit of the OLEDs produced in Device Examples 1 and 2 and Comparative Examples 1 and 2 are shown in Table 1 below.

TABLE 1

| | | | Driving | Luminous | Light- |
|---|---|---|---|---|---|
| | First | Second | Voltage | Efficiency | Emitting |
| | Host | Host | [V] | [cd/A] | Color |
| Device | C-1 | RH-1 | 3.4 | 35.0 | Red |
| Example 1 | | | | | |
| Device | C-2 | RH-1 | 2.9 | 35.3 | Red |
| Example 2 | | | | | |
| Comparative | — | RH-1 | 3.5 | 31.8 | Red |
| Example 1 | | | | | |
| Comparative | A | RH-1 | 3.4 | 33.1 | Red |
| Example 2 | | | | | |

The compounds used in the Device Examples and the Comparative Examples are shown in Table 2 below.

TABLE 2

| | |
|---|---|
| Hole Injection Layer/ Hole Transport Layer | 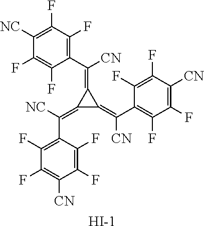 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 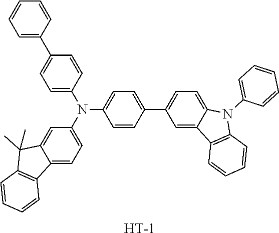 |
| | 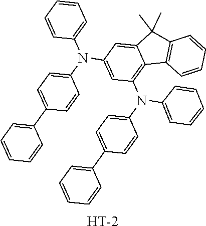 |
| Light- Emitting Layer | 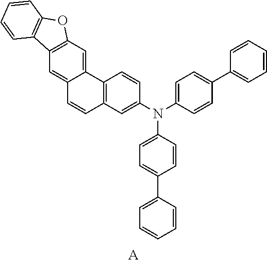 |
| | 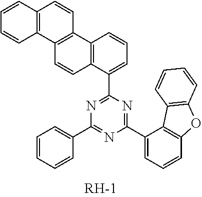 [SMILES: c1ccc(-c2nc(-c3cccc4c3ccc3c5ccccc5ccc43)nc(-c3cccc4oc5ccccc5c34)n2)cc1] |
| | 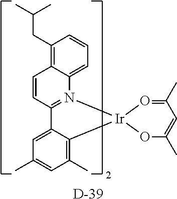 |
| | 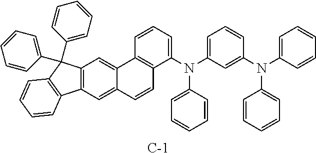 |
| | 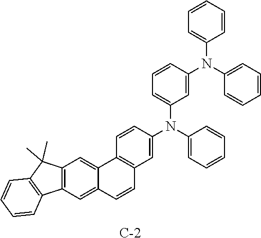 |
| Electron Transport Layer/Electron Injection Layer | 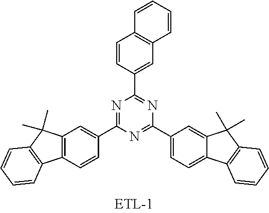 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 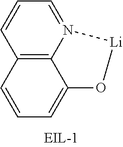 |

1. An organic electroluminescent compound represented by the following formula 1: 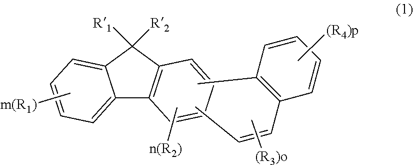
in formula 1,
R′₁ and R′₂, each independently, represent a substituted or unsubstituted (C1-C30)alkyl, a substituted or unsubstituted (C6-C30)aryl, or a substituted or unsubstituted (3- to 30-membered)heteroaryl; or R′₁ and R′₂ may be linked to each other to form a ring(s); and R′₁ and R′₂ may be the same as or different from each other;
R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L₁-N(Ar₁)-L₂-N—(Ar₂)(Ar₃); or may be linked to an adjacent substituent(s) to form a ring(s);
with a proviso that any one of R₁'s to R₄'s represents -L₁-N(Ar₁)-L₂-N—(Ar₂)(Ar₃);
L₁ represents a single bond, a substituted or unsubstituted (C6-C30)arylene, or a substituted or unsubstituted (3- to 30-membered)heteroarylene;
L₂ represents a substituted or unsubstituted (C6-C30)arylene, or a substituted or unsubstituted (3- to 30-membered)heteroarylene;
Ar₁ to Ar₃, each independently, represent a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl; and
m and p, each independently, represent an integer of 4; n and o, each independently, represent an integer of 2; and each of R₁ to each of R₄ may be the same as or different from each other.

2. The organic electroluminescent compound according to claim 1, wherein formula 1 is represented by the following formula 1-1: 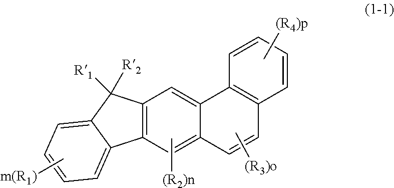
in formula 1-1, R′₁, R′₂, R₁ to R₄, and m to p are as defined in claim 1.

3. The organic electroluminescent compound according to claim 1, wherein formula 1 is represented by any one of the following formulas 1-1-1 to 1-1-4: 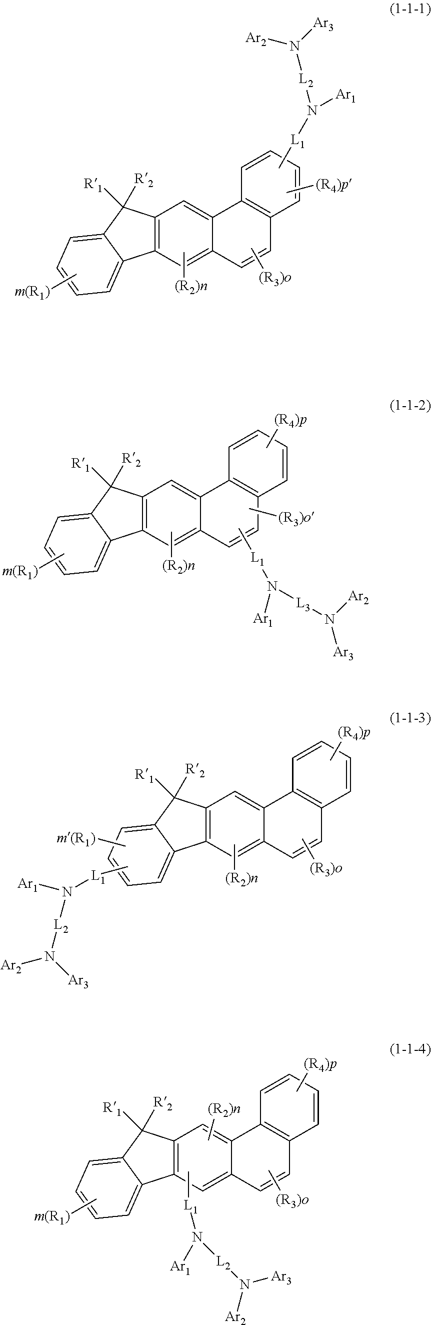
in formulas 1-1-1 to 1-1-4,
R′₁, R′₂, L₁, L₂, Ar₁ to Ar₃, and m to p are as defined in claim 1;
m′ and p′, each independently, represent an integer of 3, and n′ and o′, each independently, represent an integer of 1; and
R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s); or may be linked to an adjacent substituent(s) to form a ring(s).

4. The organic electroluminescent compound according to claim 1, wherein formula 1 is represented by any one of the following formulas 1-1-1-1 to 1-1-1-12: 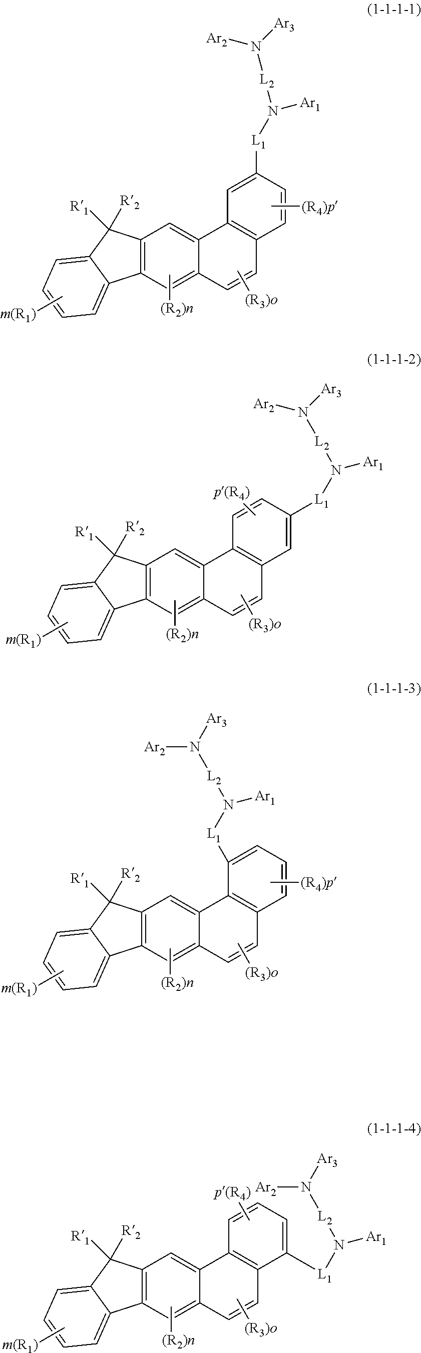 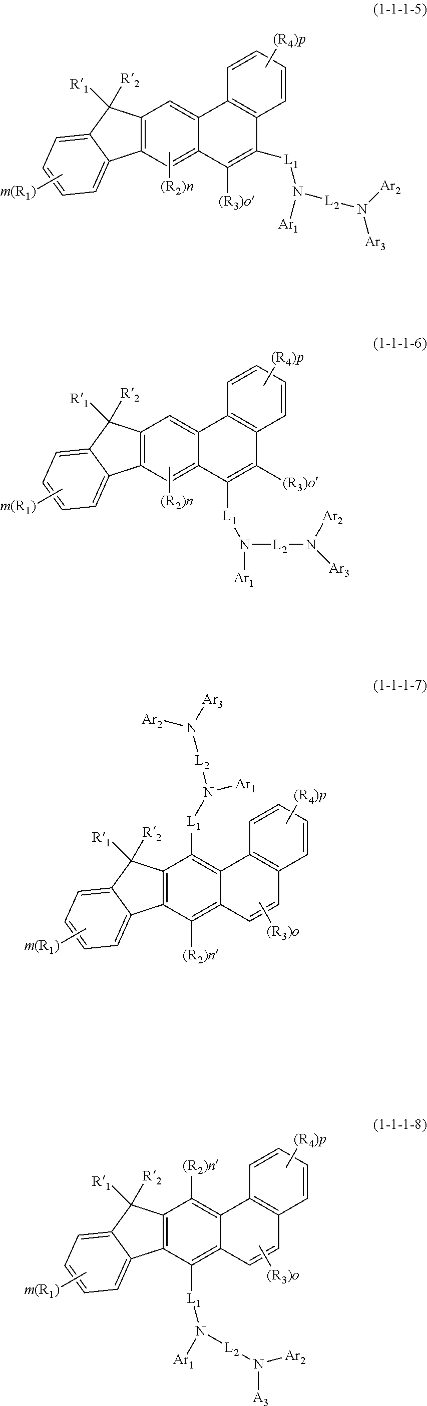 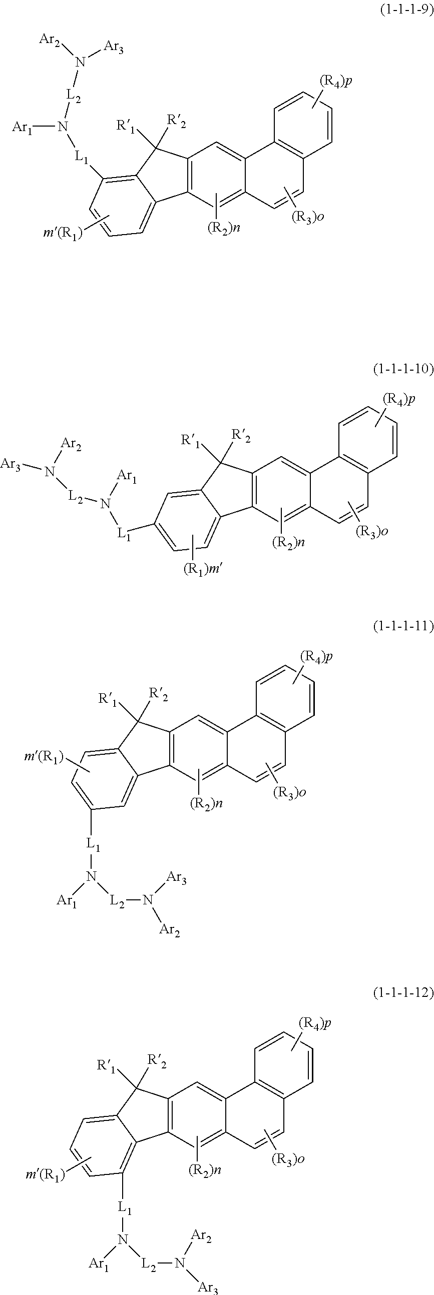
in formulas 1-1-1-1 to 1-1-1-12,
R′₁, R′₂, L₁, L₂, Ar₁ to Ar₃, and m to p are as defined in claim 1;
m′ and p′, each independently, represent an integer of 3, and n′ and o′, each independently, represent an integer of 1; and
R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s); or may be linked to an adjacent substituent(s) to form a ring(s).

5. The organic electroluminescent compound according to claim 1, wherein the substituent(s) of the substituted alkyl, the substituted alkenyl, the substituted aryl, the substituted arylene, the substituted heteroaryl, the substituted heteroarylene, the substituted cycloalkyl, the substituted alkoxy, the substituted trialkylsilyl, the substituted dialkylarylsilyl, the substituted alkyldiarylsilyl, the substituted triarylsilyl, and the substituted fused ring group of an aliphatic ring(s) and an aromatic ring(s), each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl unsubstituted or substituted with a (C6-C30)aryl(s); a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with at least one of a (C1-C30)alkyl(s) and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di- (C1-C30)alkylamino; a mono- or di- (C2-C30)alkenylamino; a mono- or di- (C6-C30)arylamino unsubstituted or substituted with a (C1-C30)alkyl(s); a mono- or di- (3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphinyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

6. The organic electroluminescent compound according to claim 1, wherein the compound represented by formula 1 is selected from the group consisting of the following compounds:

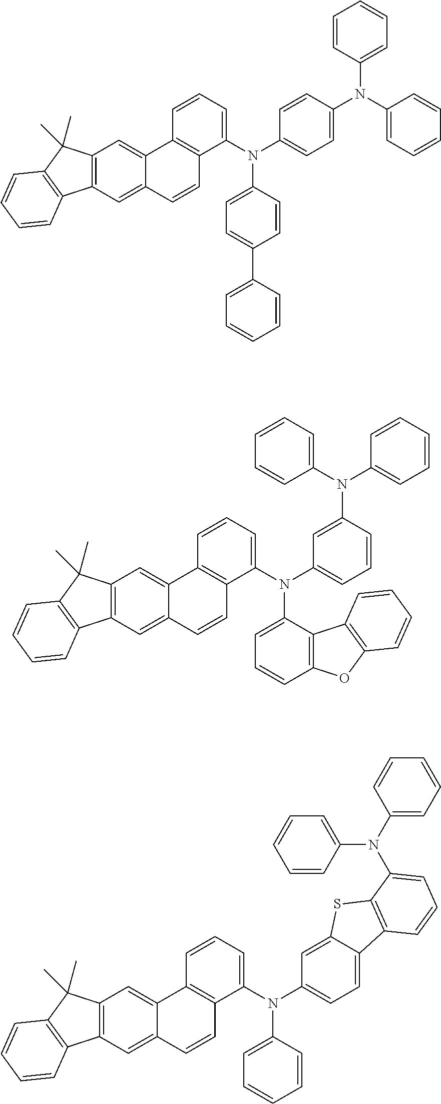 [SMILES: CC1(C)c2ccccc2-c2cc3ccc4c(N(c5ccc(-c6ccccc6)cc5)c5ccc(N(c6ccccc6)c6ccccc6)cc5)cccc4c3cc21 ; CC1(C)c2ccccc2-c2cc3ccc4c(N(c5cccc(N(c6ccccc6)c6ccccc6)c5)c5cccc6oc7ccccc7c56)cccc4c3cc21 ; CC1(C)c2ccccc2-c2cc3ccc4c(N(c5ccccc5)c5ccc6c(c5)sc5c(N(c7ccccc7)c7ccccc7)cccc56)cccc4c3cc21]

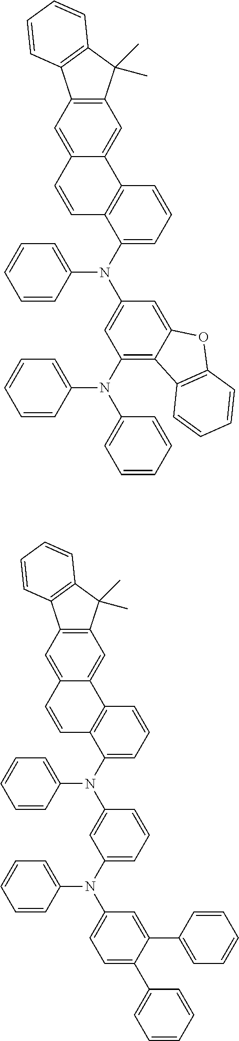 [SMILES: CC1(C)c2ccccc2-c2cc3ccc4c(N(c5ccccc5)c5cc(N(c6ccccc6)c6ccccc6)c6c(c5)oc5ccccc56)cccc4c3cc21 ; CC1(C)c2ccccc2-c2cc3ccc4c(N(c5ccccc5)c5cccc(N(c6ccccc6)c6ccc(-c7ccccc7)c(-c7ccccc7)c6)c5)cccc4c3cc21]

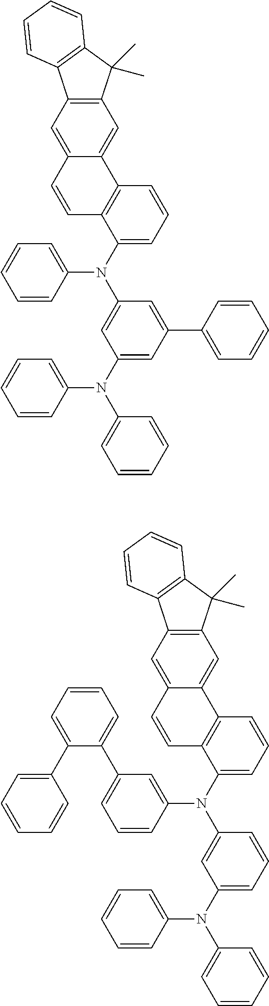 [SMILES: CC1(C)c2ccccc2-c2cc3ccc4c(N(c5ccccc5)c5cc(-c6ccccc6)cc(N(c6ccccc6)c6ccccc6)c5)cccc4c3cc21 ; CC1(C)c2ccccc2-c2cc3ccc4c(N(c5cccc(-c6ccccc6-c6ccccc6)c5)c5cccc(N(c6ccccc6)c6ccccc6)c5)cccc4c3cc21]

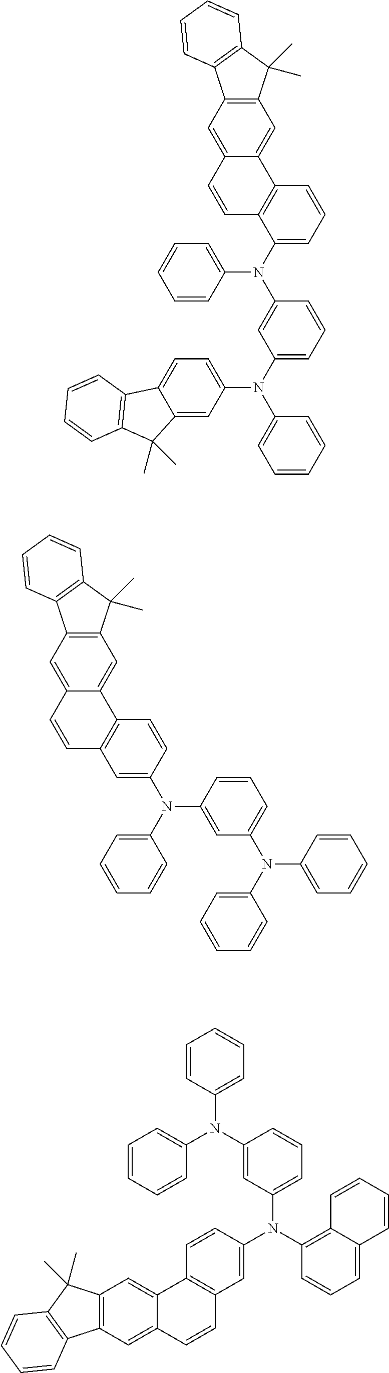 [SMILES: CC1(C)c2ccccc2-c2ccc(N(c3ccccc3)c3cccc(N(c4ccccc4)c4cccc5c4ccc4cc6c(cc45)C(C)(C)c4ccccc4-6)c3)cc21 ; CC1(C)c2ccccc2-c2cc3ccc4cc(N(c5ccccc5)c5cccc(N(c6ccccc6)c6ccccc6)c5)ccc4c3cc21 ; CC1(C)c2ccccc2-c2cc3ccc4cc(N(c5cccc(N(c6ccccc6)c6ccccc6)c5)c5cccc6ccccc56)ccc4c3cc21]

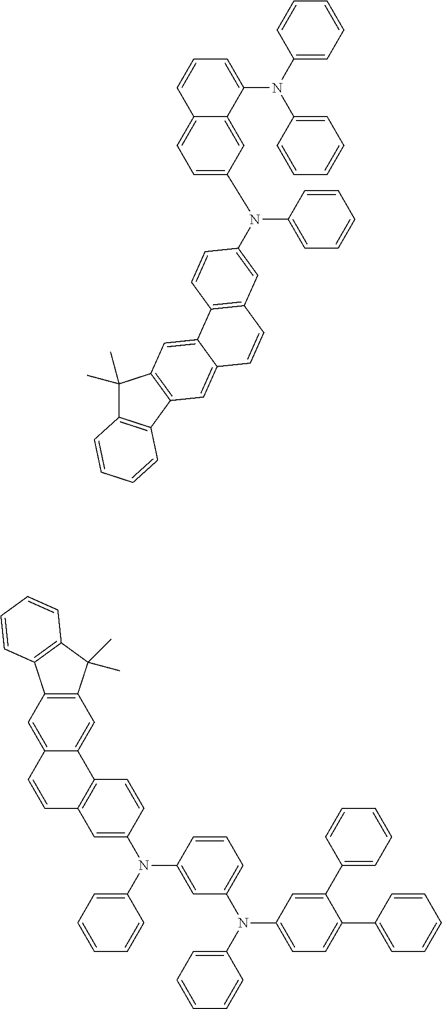 [SMILES: CC1(C)c2ccccc2-c2cc3ccc4cc(N(c5ccccc5)c5ccc6cccc(N(c7ccccc7)c7ccccc7)c6c5)ccc4c3cc21 ; CC1(C)c2ccccc2-c2cc3ccc4cc(N(c5ccccc5)c5cccc(N(c6ccccc6)c6ccc(-c7ccccc7)c(-c7ccccc7)c6)c5)ccc4c3cc21]

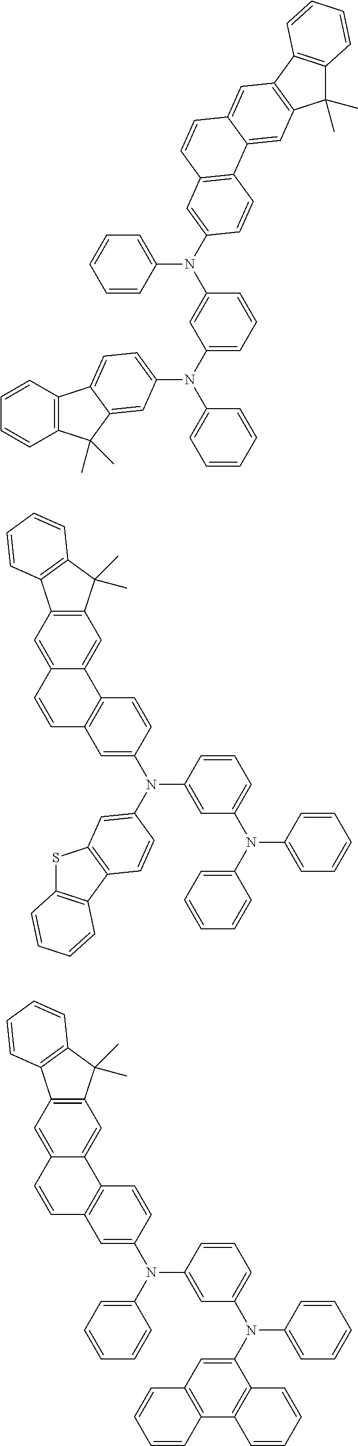 [SMILES: CC1(C)c2ccccc2-c2ccc(N(c3ccccc3)c3cccc(N(c4ccccc4)c4ccc5c(ccc6cc7c(cc65)C(C)(C)c5ccccc5-7)c4)c3)cc21 ; CC1(C)c2ccccc2-c2cc3ccc4cc(N(c5cccc(N(c6ccccc6)c6ccccc6)c5)c5ccc6c(c5)sc5ccccc56)ccc4c3cc21 ; CC1(C)c2ccccc2-c2cc3ccc4cc(N(c5ccccc5)c5cccc(N(c6ccccc6)c6cc7ccccc7c7ccccc67)c5)ccc4c3cc21]

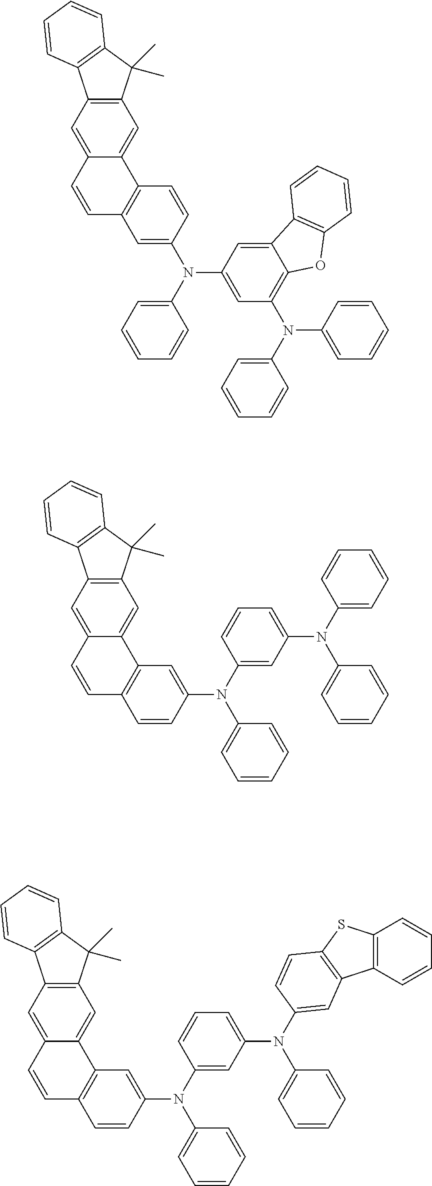 [SMILES: CC1(C)c2ccccc2-c2cc3ccc4cc(N(c5ccccc5)c5cc(N(c6ccccc6)c6ccccc6)c6oc7ccccc7c6c5)ccc4c3cc21 ; CC1(C)c2ccccc2-c2cc3ccc4ccc(N(c5ccccc5)c5cccc(N(c6ccccc6)c6ccccc6)c5)cc4c3cc21 ; CC1(C)c2ccccc2-c2cc3ccc4ccc(N(c5ccccc5)c5cccc(N(c6ccccc6)c6ccc7sc8ccccc8c7c6)c5)cc4c3cc21]

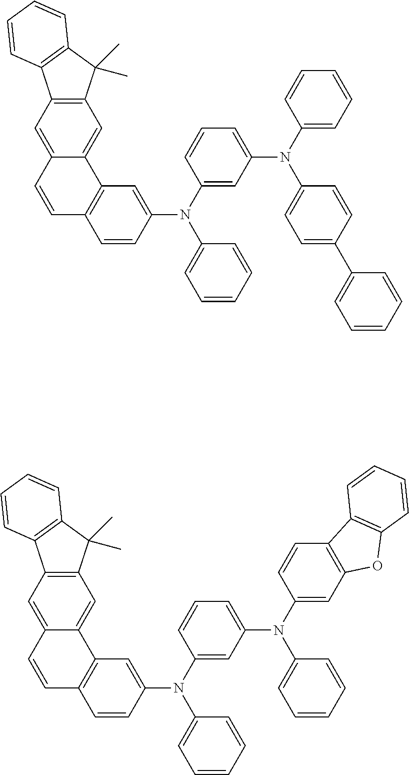 [SMILES: CC1(C)c2ccccc2-c2cc3ccc4ccc(N(c5ccccc5)c5cccc(N(c6ccccc6)c6ccc(-c7ccccc7)cc6)c5)cc4c3cc21 ; CC1(C)c2ccccc2-c2cc3ccc4ccc(N(c5ccccc5)c5cccc(N(c6ccccc6)c6ccc7c(c6)oc6ccccc67)c5)cc4c3cc21]

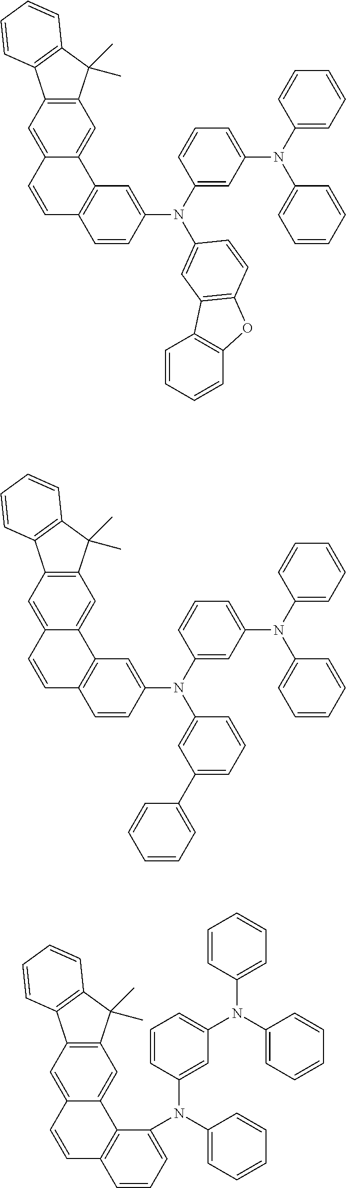 [SMILES: CC1(C)c2ccccc2-c2cc3ccc4ccc(N(c5cccc(N(c6ccccc6)c6ccccc6)c5)c5ccc6oc7ccccc7c6c5)cc4c3cc21 ; CC1(C)c2ccccc2-c2cc3ccc4ccc(N(c5cccc(-c6ccccc6)c5)c5cccc(N(c6ccccc6)c6ccccc6)c5)cc4c3cc21 ; CC1(C)c2ccccc2-c2cc3ccc4cccc(N(c5ccccc5)c5cccc(N(c6ccccc6)c6ccccc6)c5)c4c3cc21]

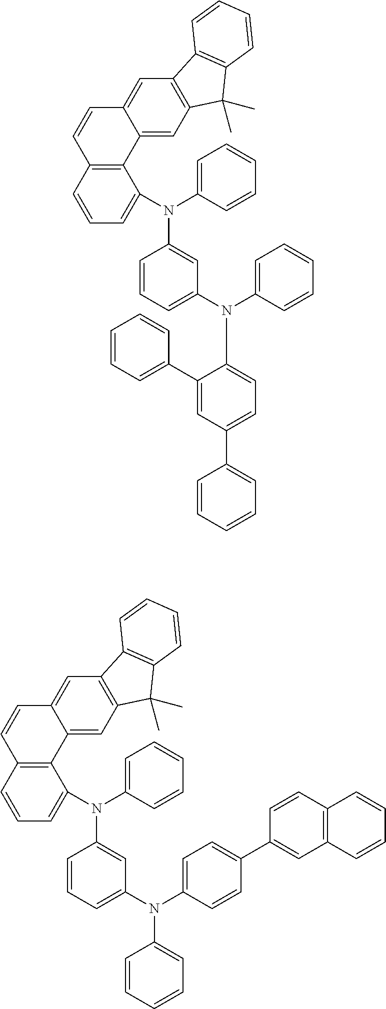 [SMILES: CC1(C)c2ccccc2-c2cc3ccc4cccc(N(c5ccccc5)c5cccc(N(c6ccccc6)c6ccc(-c7ccccc7)cc6-c6ccccc6)c5)c4c3cc21 ; CC1(C)c2ccccc2-c2cc3ccc4cccc(N(c5ccccc5)c5cccc(N(c6ccccc6)c6ccc(-c7ccc8ccccc8c7)cc6)c5)c4c3cc21]

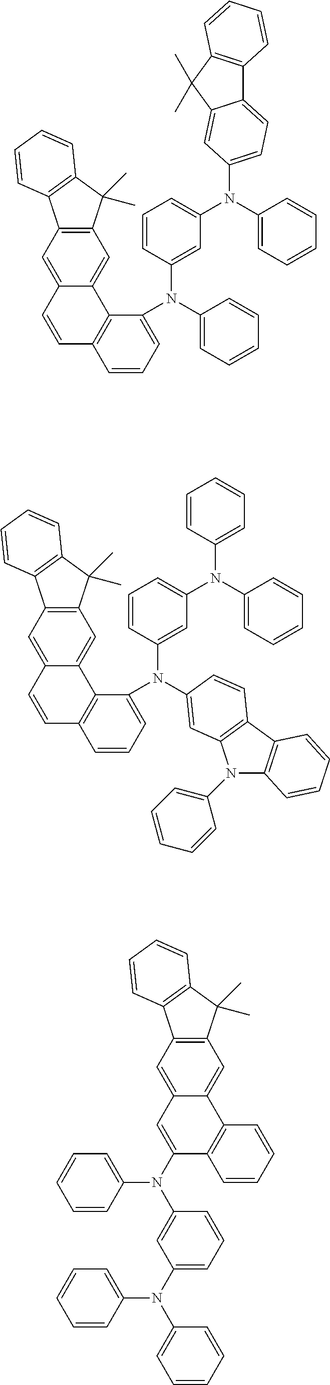 [SMILES: CC1(C)c2ccccc2-c2ccc(N(c3ccccc3)c3cccc(N(c4ccccc4)c4cccc5ccc6cc7c(cc6c45)C(C)(C)c4ccccc4-7)c3)cc21 ; CC1(C)c2ccccc2-c2cc3ccc4cccc(N(c5cccc(N(c6ccccc6)c6ccccc6)c5)c5ccc6c7ccccc7n(-c7ccccc7)c6c5)c4c3cc21 ; CC1(C)c2ccccc2-c2cc3cc(N(c4ccccc4)c4cccc(N(c5ccccc5)c5ccccc5)c4)c4ccccc4c3cc21]

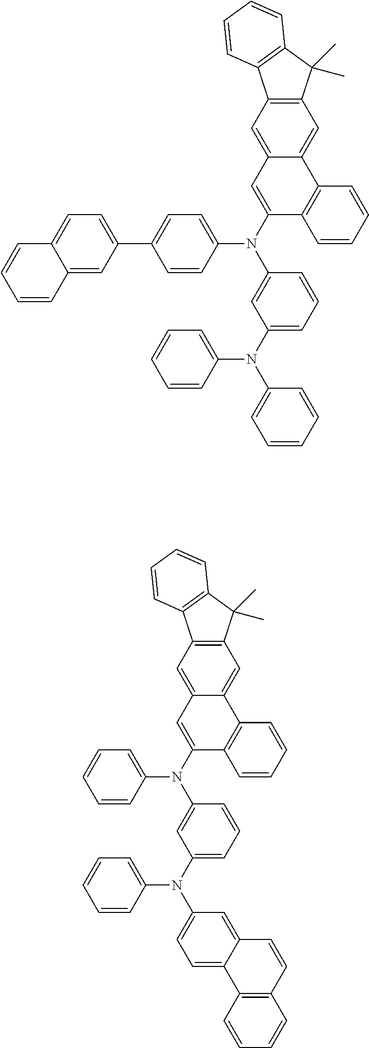 [SMILES: CC1(C)c2ccccc2-c2cc3cc(N(c4ccc(-c5ccc6ccccc6c5)cc4)c4cccc(N(c5ccccc5)c5ccccc5)c4)c4ccccc4c3cc21 ; CC1(C)c2ccccc2-c2cc3cc(N(c4ccccc4)c4cccc(N(c5ccccc5)c5ccc6c(ccc7ccccc76)c5)c4)c4ccccc4c3cc21]

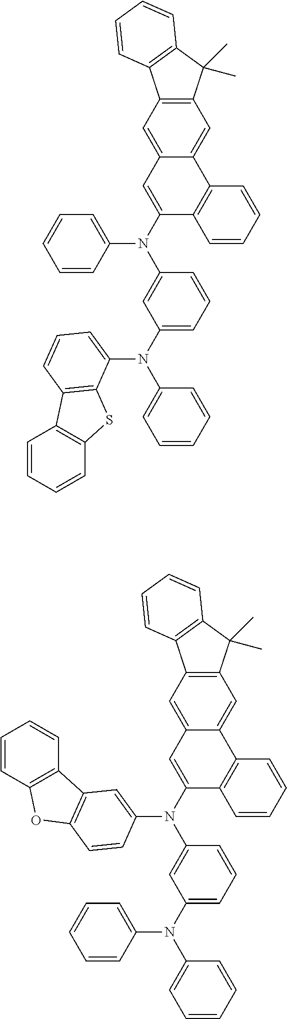 [SMILES: CC1(C)c2ccccc2-c2cc3cc(N(c4ccccc4)c4cccc(N(c5ccccc5)c5cccc6c5sc5ccccc56)c4)c4ccccc4c3cc21 ; CC1(C)c2ccccc2-c2cc3cc(N(c4cccc(N(c5ccccc5)c5ccccc5)c4)c4ccc5oc6ccccc6c5c4)c4ccccc4c3cc21]

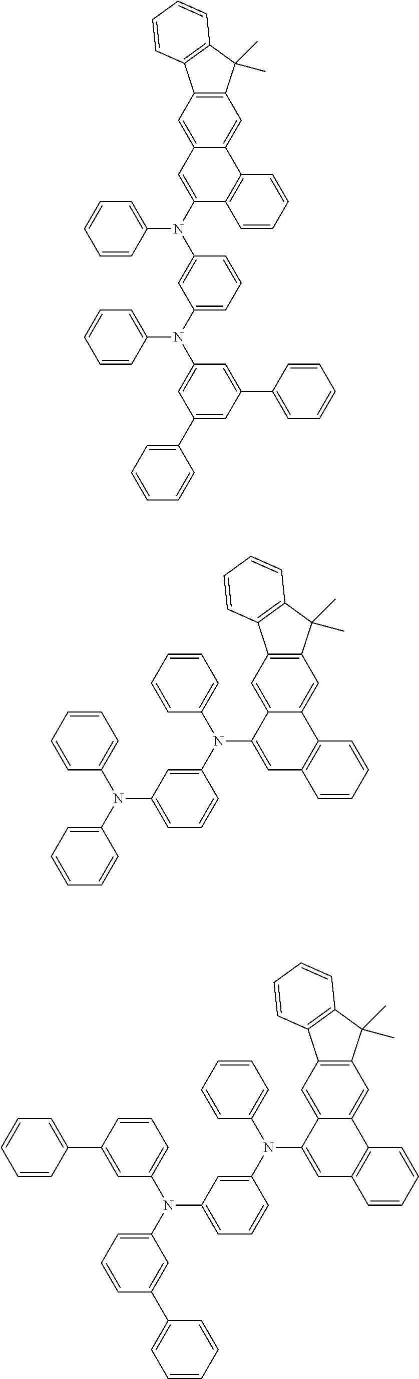 [SMILES: CC1(C)c2ccccc2-c2cc3cc(N(c4ccccc4)c4cccc(N(c5ccccc5)c5cc(-c6ccccc6)cc(-c6ccccc6)c5)c4)c4ccccc4c3cc21 ; CC1(C)c2ccccc2-c2cc3c(N(c4ccccc4)c4cccc(N(c5ccccc5)c5ccccc5)c4)cc4ccccc4c3cc21 ; CC1(C)c2ccccc2-c2cc3c(N(c4ccccc4)c4cccc(N(c5cccc(-c6ccccc6)c5)c5cccc(-c6ccccc6)c5)c4)cc4ccccc4c3cc21]

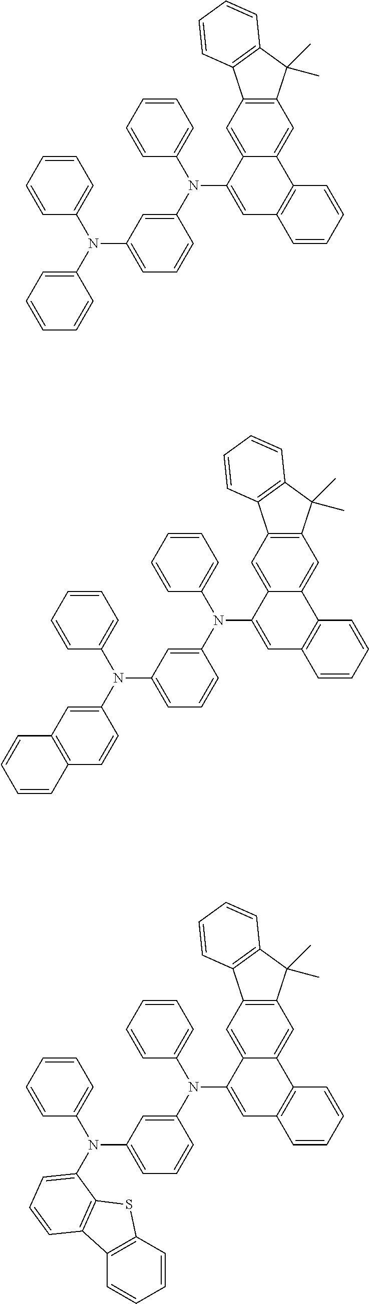 [SMILES: CC1(C)c2ccccc2-c2cc3c(N(c4ccccc4)c4cccc(N(c5ccccc5)c5ccccc5)c4)cc4ccccc4c3cc21 ; CC1(C)c2ccccc2-c2cc3c(N(c4ccccc4)c4cccc(N(c5ccccc5)c5ccc6ccccc6c5)c4)cc4ccccc4c3cc21 ; CC1(C)c2ccccc2-c2cc3c(N(c4ccccc4)c4cccc(N(c5ccccc5)c5cccc6c5sc5ccccc56)c4)cc4ccccc4c3cc21]

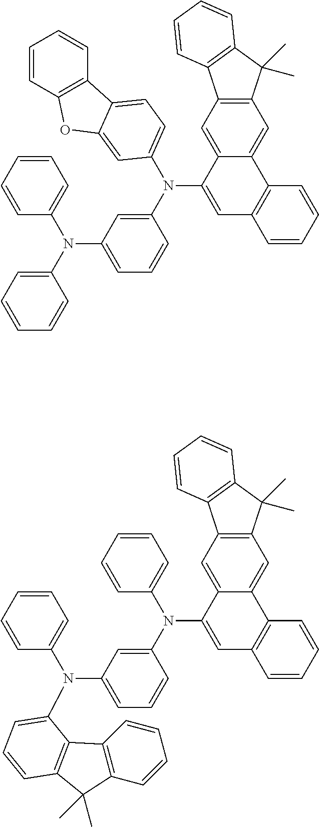 [SMILES: CC1(C)c2ccccc2-c2cc3c(N(c4cccc(N(c5ccccc5)c5ccccc5)c4)c4ccc5c(c4)oc4ccccc45)cc4ccccc4c3cc21 ; CC1(C)c2ccccc2-c2cc3c(N(c4ccccc4)c4cccc(N(c5ccccc5)c5cccc6c5-c5ccccc5C6(C)C)c4)cc4ccccc4c3cc21]

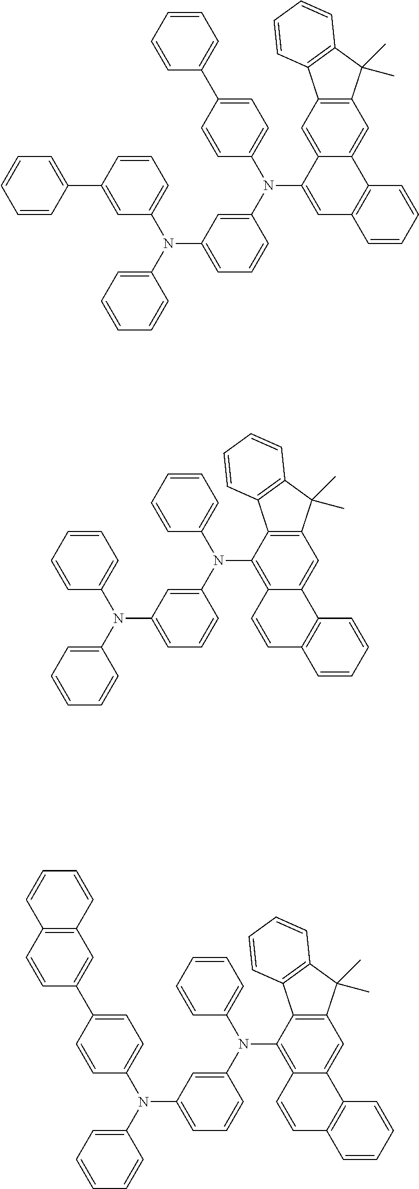 [SMILES: CC1(C)c2ccccc2-c2cc3c(N(c4ccc(-c5ccccc5)cc4)c4cccc(N(c5ccccc5)c5cccc(-c6ccccc6)c5)c4)cc4ccccc4c3cc21 ; CC1(C)c2ccccc2-c2c1cc1c(ccc3ccccc31)c2N(c1ccccc1)c1cccc(N(c2ccccc2)c2ccccc2)c1 ; CC1(C)c2ccccc2-c2c1cc1c(ccc3ccccc31)c2N(c1ccccc1)c1cccc(N(c2ccccc2)c2ccc(-c3ccc4ccccc4c3)cc2)c1]

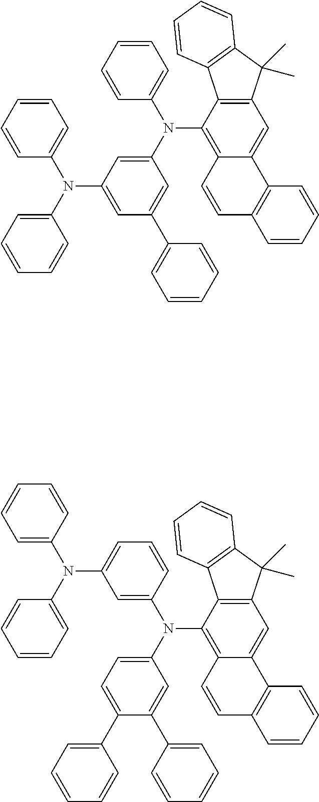 [SMILES: CC1(C)c2ccccc2-c2c1cc1c(ccc3ccccc31)c2N(c1ccccc1)c1cc(-c2ccccc2)cc(N(c2ccccc2)c2ccccc2)c1 ; CC1(C)c2ccccc2-c2c1cc1c(ccc3ccccc31)c2N(c1cccc(N(c2ccccc2)c2ccccc2)c1)c1ccc(-c2ccccc2)c(-c2ccccc2)c1]

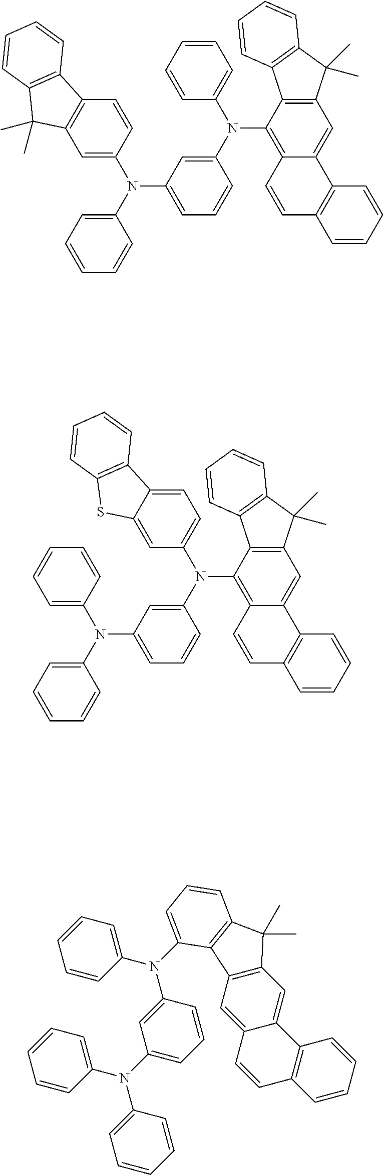 [SMILES: CC1(C)c2ccccc2-c2ccc(N(c3ccccc3)c3cccc(N(c4ccccc4)c4c5c(cc6c4ccc4ccccc46)C(C)(C)c4ccccc4-5)c3)cc21 ; CC1(C)c2ccccc2-c2c1cc1c(ccc3ccccc31)c2N(c1cccc(N(c2ccccc2)c2ccccc2)c1)c1ccc2c(c1)sc1ccccc12 ; CC1(C)c2cc3c(ccc4ccccc43)cc2-c2c(N(c3ccccc3)c3cccc(N(c4ccccc4)c4ccccc4)c3)cccc21]

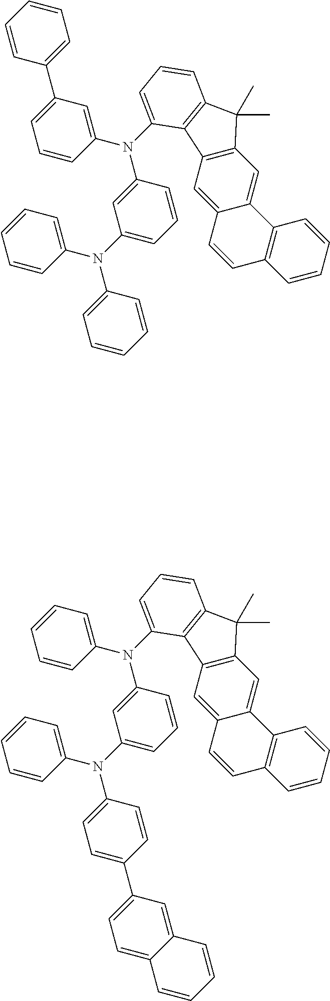 [SMILES: CC1(C)c2cc3c(ccc4ccccc43)cc2-c2c(N(c3cccc(-c4ccccc4)c3)c3cccc(N(c4ccccc4)c4ccccc4)c3)cccc21 ; CC1(C)c2cc3c(ccc4ccccc43)cc2-c2c(N(c3ccccc3)c3cccc(N(c4ccccc4)c4ccc(-c5ccc6ccccc6c5)cc4)c3)cccc21]

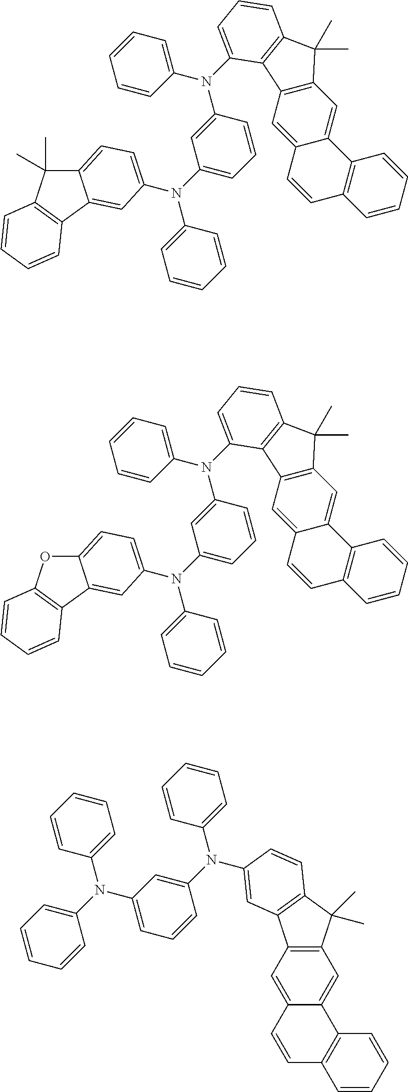 [SMILES: CC1(C)c2ccccc2-c2cc(N(c3ccccc3)c3cccc(N(c4ccccc4)c4cccc5c4-c4cc6ccc7ccccc7c6cc4C5(C)C)c3)ccc21 ; CC1(C)c2cc3c(ccc4ccccc43)cc2-c2c(N(c3ccccc3)c3cccc(N(c4ccccc4)c4ccc5oc6ccccc6c5c4)c3)cccc21 ; CC1(C)c2ccc(N(c3ccccc3)c3cccc(N(c4ccccc4)c4ccccc4)c3)cc2-c2cc3ccc4ccccc4c3cc21]

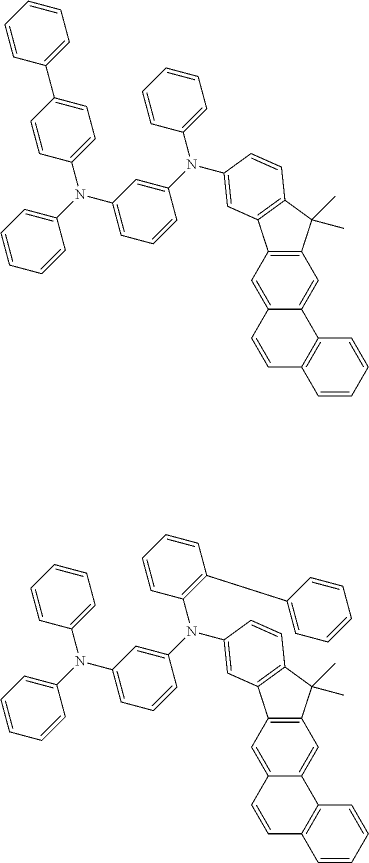 [SMILES: CC1(C)c2ccc(N(c3ccccc3)c3cccc(N(c4ccccc4)c4ccc(-c5ccccc5)cc4)c3)cc2-c2cc3ccc4ccccc4c3cc21 ; CC1(C)c2ccc(N(c3cccc(N(c4ccccc4)c4ccccc4)c3)c3ccccc3-c3ccccc3)cc2-c2cc3ccc4ccccc4c3cc21]

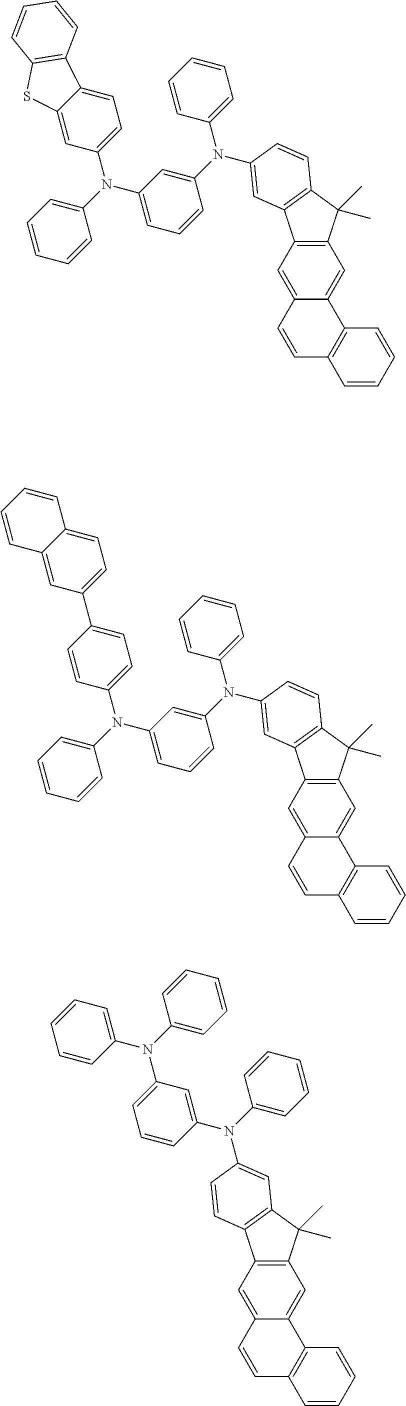 [SMILES: CC1(C)c2ccc(N(c3ccccc3)c3cccc(N(c4ccccc4)c4ccc5c(c4)sc4ccccc45)c3)cc2-c2cc3ccc4ccccc4c3cc21 ; CC1(C)c2ccc(N(c3ccccc3)c3cccc(N(c4ccccc4)c4ccc(-c5ccc6ccccc6c5)cc4)c3)cc2-c2cc3ccc4ccccc4c3cc21 ; CC1(C)c2cc(N(c3ccccc3)c3cccc(N(c4ccccc4)c4ccccc4)c3)ccc2-c2cc3ccc4ccccc4c3cc21]

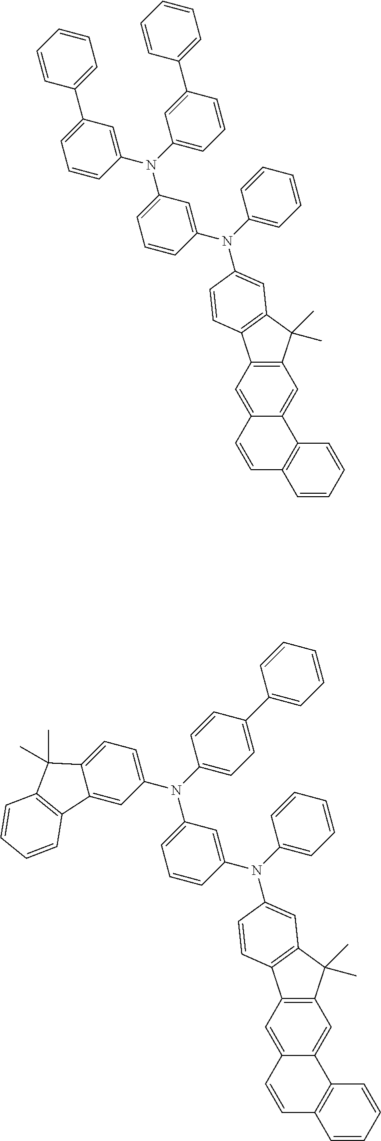 [SMILES: CC1(C)c2cc(N(c3ccccc3)c3cccc(N(c4cccc(-c5ccccc5)c4)c4cccc(-c5ccccc5)c4)c3)ccc2-c2cc3ccc4ccccc4c3cc21 ; CC1(C)c2ccccc2-c2cc(N(c3ccc(-c4ccccc4)cc3)c3cccc(N(c4ccccc4)c4ccc5c(c4)C(C)(C)c4cc6c(ccc7ccccc76)cc4-5)c3)ccc21]

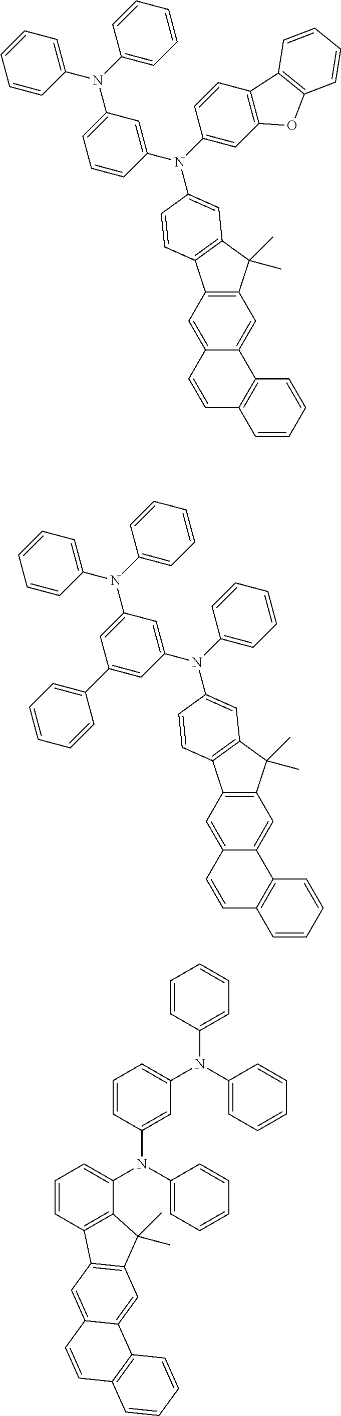 [SMILES: CC1(C)c2cc(N(c3cccc(N(c4ccccc4)c4ccccc4)c3)c3ccc4c(c3)oc3ccccc34)ccc2-c2cc3ccc4ccccc4c3cc21 ; CC1(C)c2cc(N(c3ccccc3)c3cc(-c4ccccc4)cc(N(c4ccccc4)c4ccccc4)c3)ccc2-c2cc3ccc4ccccc4c3cc21 ; CC1(C)c2cc3c(ccc4ccccc43)cc2-c2cccc(N(c3ccccc3)c3cccc(N(c4ccccc4)c4ccccc4)c3)c21]

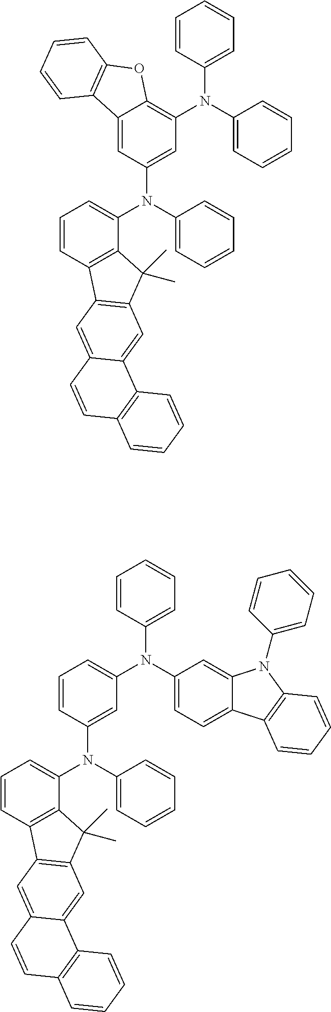 [SMILES: CC1(C)c2cc3c(ccc4ccccc43)cc2-c2cccc(N(c3ccccc3)c3cc(N(c4ccccc4)c4ccccc4)c4oc5ccccc5c4c3)c21 ; CC1(C)c2cc3c(ccc4ccccc43)cc2-c2cccc(N(c3ccccc3)c3cccc(N(c4ccccc4)c4ccc5c6ccccc6n(-c6ccccc6)c5c4)c3)c21]

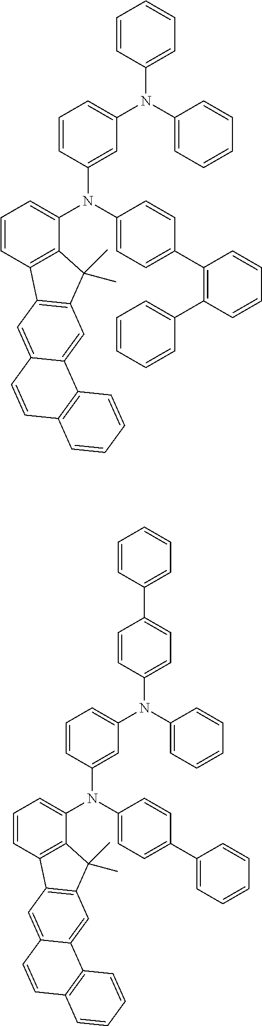 [SMILES: CC1(C)c2cc3c(ccc4ccccc43)cc2-c2cccc(N(c3ccc(-c4ccccc4-c4ccccc4)cc3)c3cccc(N(c4ccccc4)c4ccccc4)c3)c21 ; CC1(C)c2cc3c(ccc4ccccc43)cc2-c2cccc(N(c3ccc(-c4ccccc4)cc3)c3cccc(N(c4ccccc4)c4ccc(-c5ccccc5)cc4)c3)c21]

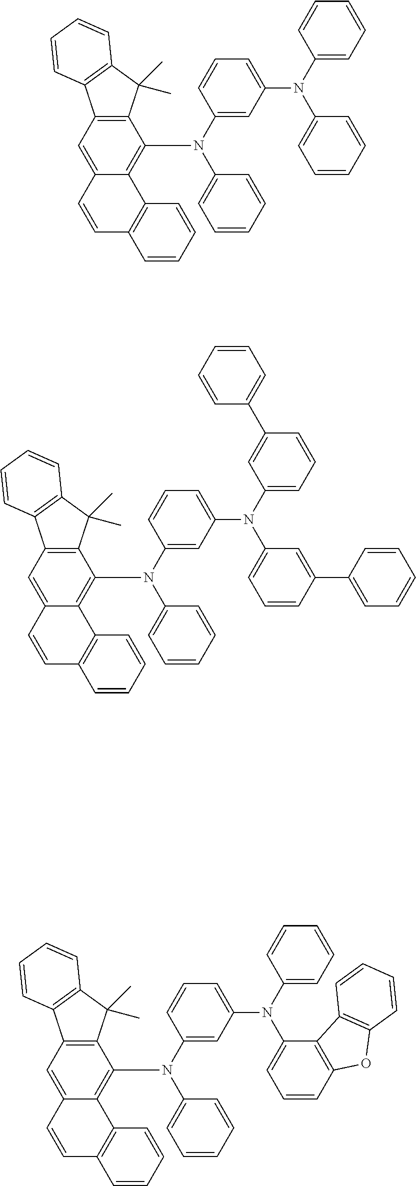 [SMILES: CC1(C)c2ccccc2-c2cc3ccc4ccccc4c3c(N(c3ccccc3)c3cccc(N(c4ccccc4)c4ccccc4)c3)c21 ; CC1(C)c2ccccc2-c2cc3ccc4ccccc4c3c(N(c3ccccc3)c3cccc(N(c4cccc(-c5ccccc5)c4)c4cccc(-c5ccccc5)c4)c3)c21 ; CC1(C)c2ccccc2-c2cc3ccc4ccccc4c3c(N(c3ccccc3)c3cccc(N(c4ccccc4)c4cccc5oc6ccccc6c45)c3)c21]

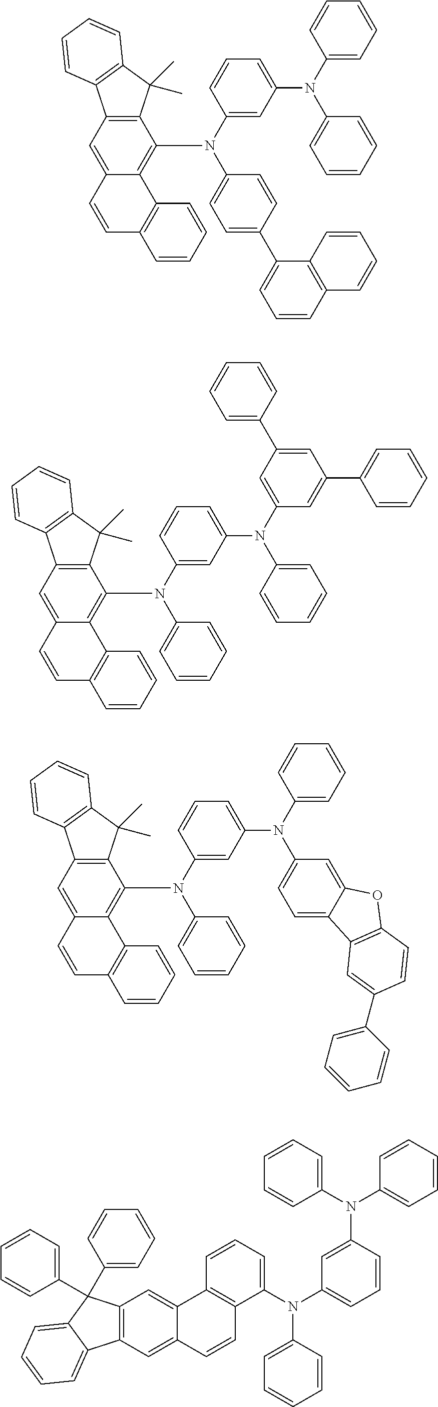

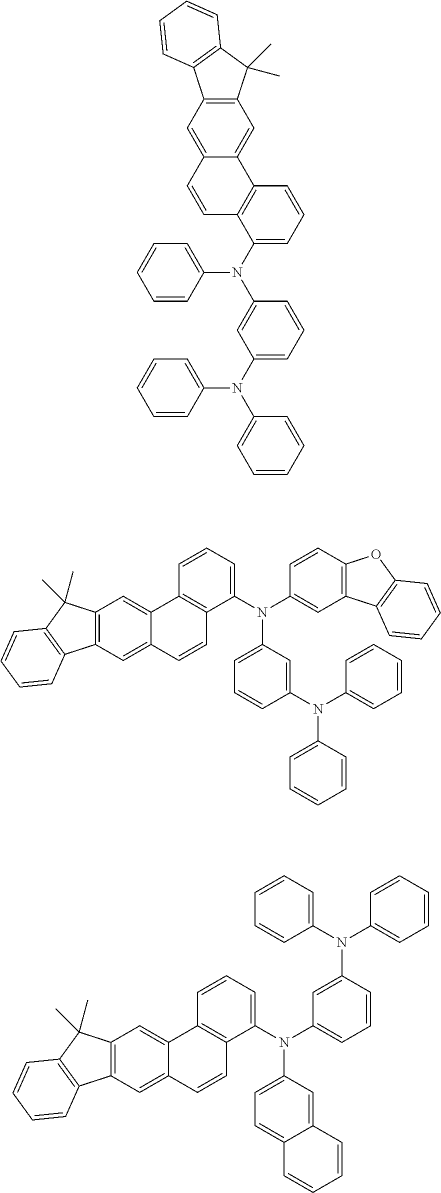 [SMILES: CC1(C)c2ccccc2-c2cc3ccc4c(N(c5ccccc5)c5cccc(N(c6ccccc6)c6ccccc6)c5)cccc4c3cc21 ; CC1(C)c2ccccc2-c2cc3ccc4c(N(c5cccc(N(c6ccccc6)c6ccccc6)c5)c5ccc6oc7ccccc7c6c5)cccc4c3cc21 ; CC1(C)c2ccccc2-c2cc3ccc4c(N(c5cccc(N(c6ccccc6)c6ccccc6)c5)c5ccc6ccccc6c5)cccc4c3cc21]

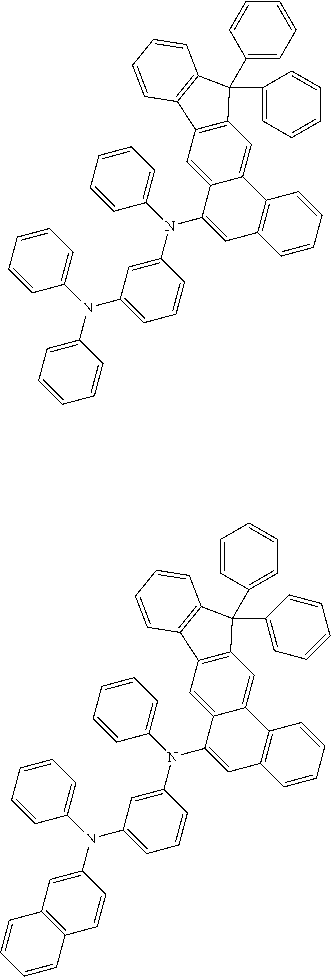 [SMILES: c1ccc(N(c2ccccc2)c2cccc(N(c3ccccc3)c3cc4ccccc4c4cc5c(cc34)-c3ccccc3C5(c3ccccc3)c3ccccc3)c2)cc1 ; c1ccc(N(c2cccc(N(c3ccccc3)c3cc4ccccc4c4cc5c(cc34)-c3ccccc3C5(c3ccccc3)c3ccccc3)c2)c2ccc3ccccc3c2)cc1]

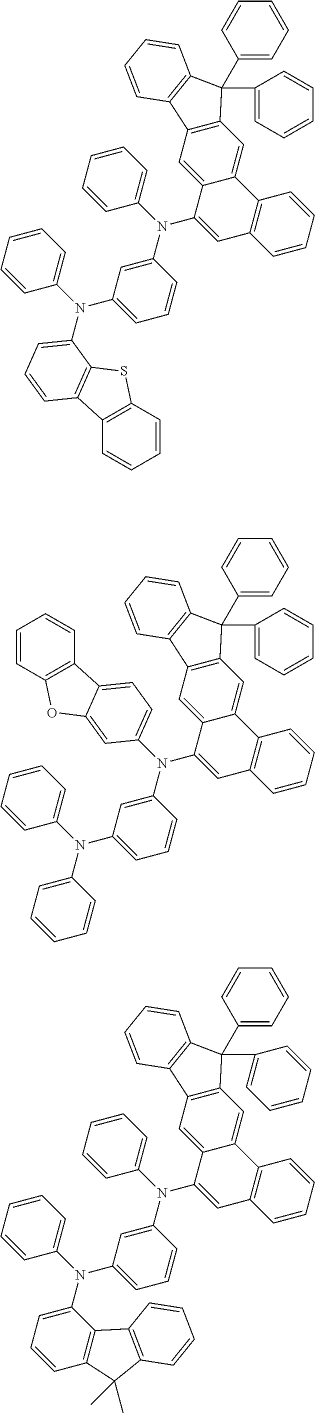 [SMILES: c1ccc(N(c2cccc(N(c3ccccc3)c3cccc4c3sc3ccccc34)c2)c2cc3ccccc3c3cc4c(cc23)-c2ccccc2C4(c2ccccc2)c2ccccc2)cc1 ; c1ccc(N(c2ccccc2)c2cccc(N(c3ccc4c(c3)oc3ccccc34)c3cc4ccccc4c4cc5c(cc34)-c3ccccc3C5(c3ccccc3)c3ccccc3)c2)cc1 ; CC1(C)c2ccccc2-c2c(N(c3ccccc3)c3cccc(N(c4ccccc4)c4cc5ccccc5c5cc6c(cc45)-c4ccccc4C6(c4ccccc4)c4ccccc4)c3)cccc21]

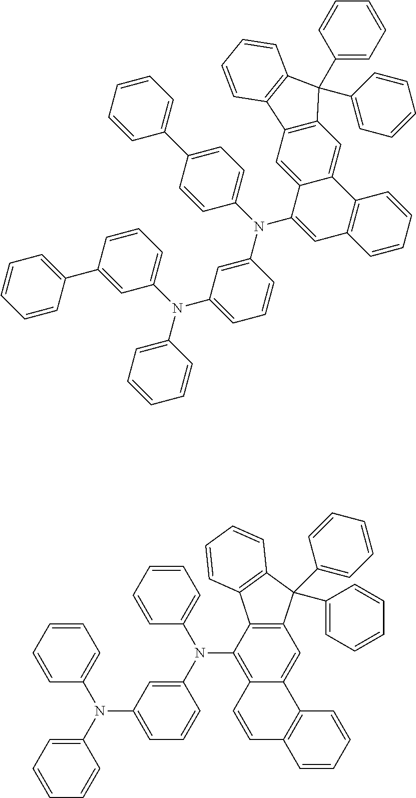 [SMILES: c1ccc(-c2ccc(N(c3cccc(N(c4ccccc4)c4cccc(-c5ccccc5)c4)c3)c3cc4ccccc4c4cc5c(cc34)-c3ccccc3C5(c3ccccc3)c3ccccc3)cc2)cc1 ; c1ccc(N(c2ccccc2)c2cccc(N(c3ccccc3)c3c4c(cc5c3ccc3ccccc35)C(c3ccccc3)(c3ccccc3)c3ccccc3-4)c2)cc1]

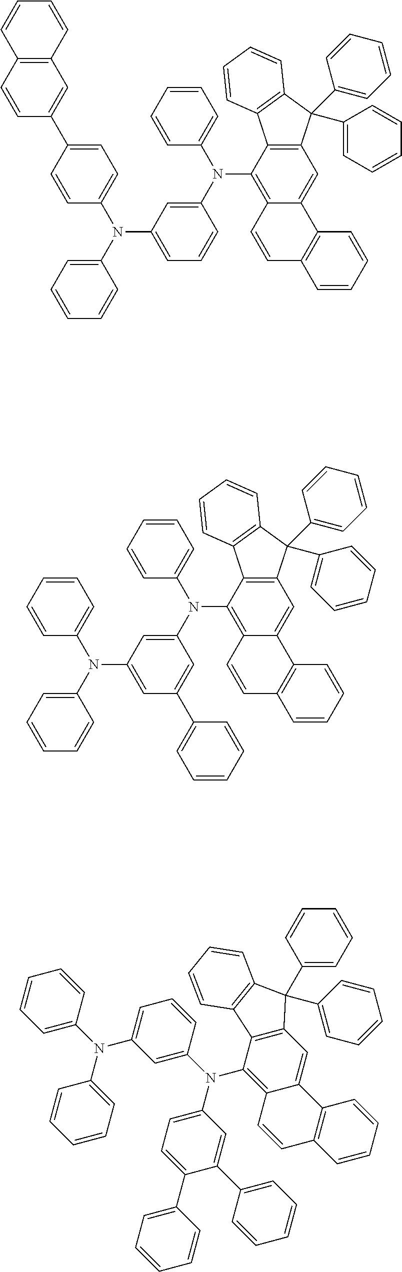 [SMILES: c1ccc(N(c2ccc(-c3ccc4ccccc4c3)cc2)c2cccc(N(c3ccccc3)c3c4c(cc5c3ccc3ccccc35)C(c3ccccc3)(c3ccccc3)c3ccccc3-4)c2)cc1 ; c1ccc(-c2cc(N(c3ccccc3)c3ccccc3)cc(N(c3ccccc3)c3c4c(cc5c3ccc3ccccc35)C(c3ccccc3)(c3ccccc3)c3ccccc3-4)c2)cc1 ; c1ccc(-c2ccc(N(c3cccc(N(c4ccccc4)c4ccccc4)c3)c3c4c(cc5c3ccc3ccccc35)C(c3ccccc3)(c3ccccc3)c3ccccc3-4)cc2-c2ccccc2)cc1]

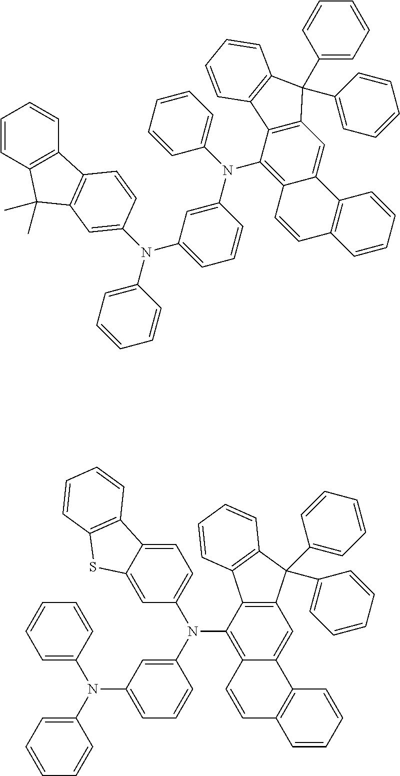 [SMILES: CC1(C)c2ccccc2-c2ccc(N(c3ccccc3)c3cccc(N(c4ccccc4)c4c5c(cc6c4ccc4ccccc46)C(c4ccccc4)(c4ccccc4)c4ccccc4-5)c3)cc21 ; c1ccc(N(c2ccccc2)c2cccc(N(c3ccc4c(c3)sc3ccccc34)c3c4c(cc5c3ccc3ccccc35)C(c3ccccc3)(c3ccccc3)c3ccccc3-4)c2)cc1]

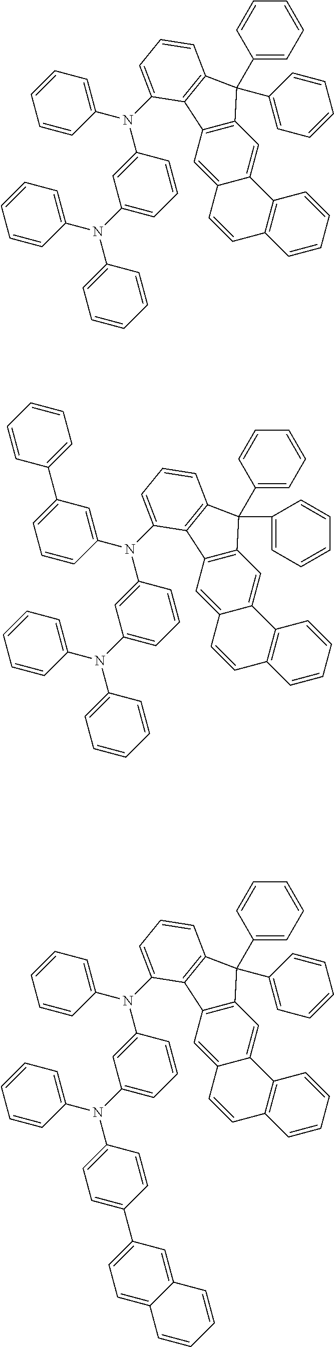 [SMILES: c1ccc(N(c2ccccc2)c2cccc(N(c3ccccc3)c3cccc4c3-c3cc5ccc6ccccc6c5cc3C4(c3ccccc3)c3ccccc3)c2)cc1 ; c1ccc(-c2cccc(N(c3cccc(N(c4ccccc4)c4ccccc4)c3)c3cccc4c3-c3cc5ccc6ccccc6c5cc3C4(c3ccccc3)c3ccccc3)c2)cc1 ; c1ccc(N(c2ccc(-c3ccc4ccccc4c3)cc2)c2cccc(N(c3ccccc3)c3cccc4c3-c3cc5ccc6ccccc6c5cc3C4(c3ccccc3)c3ccccc3)c2)cc1]

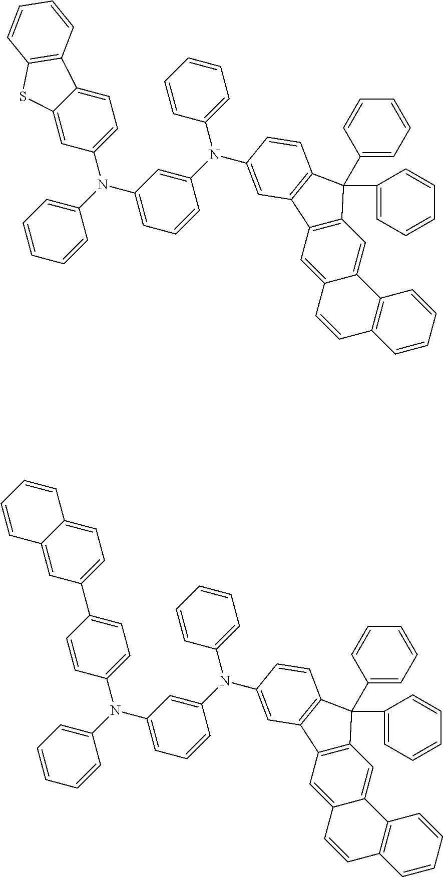 [SMILES: c1ccc(N(c2cccc(N(c3ccccc3)c3ccc4c(c3)sc3ccccc34)c2)c2ccc3c(c2)-c2cc4ccc5ccccc5c4cc2C3(c2ccccc2)c2ccccc2)cc1 ; c1ccc(N(c2ccc(-c3ccc4ccccc4c3)cc2)c2cccc(N(c3ccccc3)c3ccc4c(c3)-c3cc5ccc6ccccc6c5cc3C4(c3ccccc3)c3ccccc3)c2)cc1]

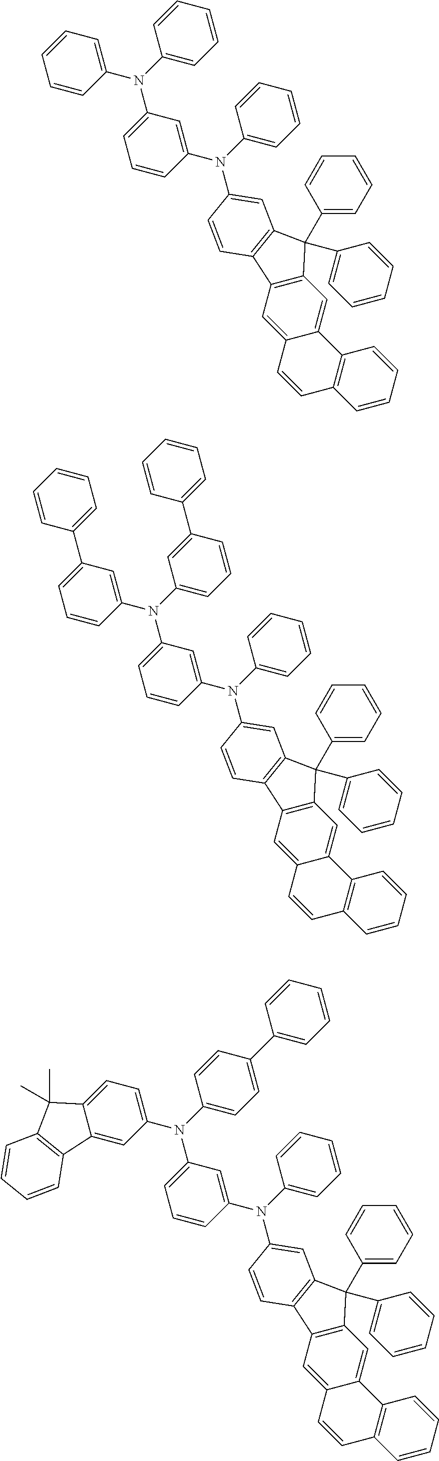 [SMILES: c1ccc(N(c2ccccc2)c2cccc(N(c3ccccc3)c3ccc4c(c3)C(c3ccccc3)(c3ccccc3)c3cc5c(ccc6ccccc65)cc3-4)c2)cc1 ; c1ccc(-c2cccc(N(c3cccc(-c4ccccc4)c3)c3cccc(N(c4ccccc4)c4ccc5c(c4)C(c4ccccc4)(c4ccccc4)c4cc6c(ccc7ccccc76)cc4-5)c3)c2)cc1 ; CC1(C)c2ccccc2-c2cc(N(c3ccc(-c4ccccc4)cc3)c3cccc(N(c4ccccc4)c4ccc5c(c4)C(c4ccccc4)(c4ccccc4)c4cc6c(ccc7ccccc76)cc4-5)c3)ccc21]

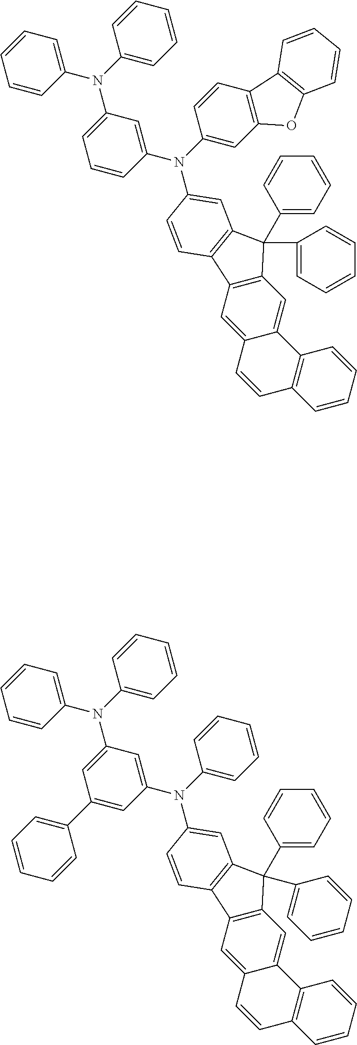 [SMILES: c1ccc(N(c2ccccc2)c2cccc(N(c3ccc4c(c3)C(c3ccccc3)(c3ccccc3)c3cc5c(ccc6ccccc65)cc3-4)c3ccc4c(c3)oc3ccccc34)c2)cc1 ; c1ccc(-c2cc(N(c3ccccc3)c3ccccc3)cc(N(c3ccccc3)c3ccc4c(c3)C(c3ccccc3)(c3ccccc3)c3cc5c(ccc6ccccc65)cc3-4)c2)cc1]

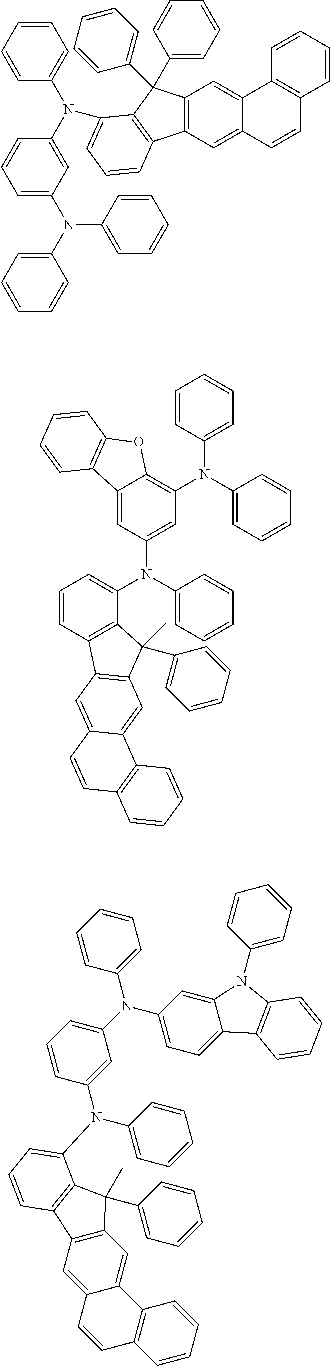 [SMILES: c1ccc(N(c2ccccc2)c2cccc(N(c3ccccc3)c3cccc4c3C(c3ccccc3)(c3ccccc3)c3cc5c(ccc6ccccc65)cc3-4)c2)cc1 ; CC1(c2ccccc2)c2cc3c(ccc4ccccc43)cc2-c2cccc(N(c3ccccc3)c3cc(N(c4ccccc4)c4ccccc4)c4oc5ccccc5c4c3)c21 ; CC1(c2ccccc2)c2cc3c(ccc4ccccc43)cc2-c2cccc(N(c3ccccc3)c3cccc(N(c4ccccc4)c4ccc5c6ccccc6n(-c6ccccc6)c5c4)c3)c21]

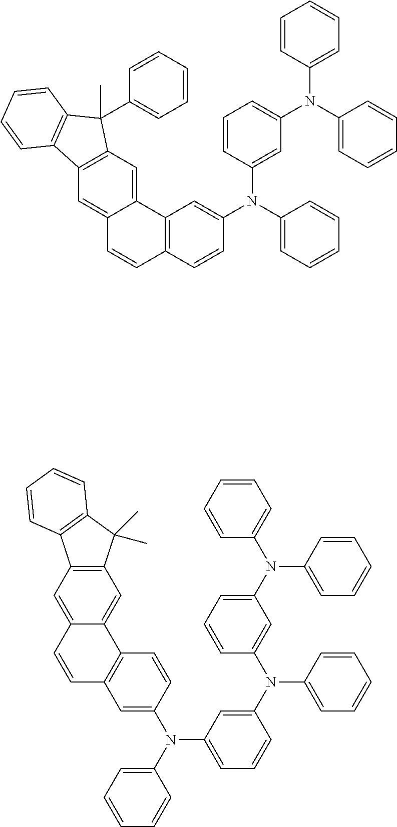 [SMILES: CC1(c2ccccc2)c2ccccc2-c2cc3ccc4ccc(N(c5ccccc5)c5cccc(N(c6ccccc6)c6ccccc6)c5)cc4c3cc21 ; CC1(C)c2ccccc2-c2cc3ccc4cc(N(c5ccccc5)c5cccc(N(c6ccccc6)c6cccc(N(c7ccccc7)c7ccccc7)c6)c5)ccc4c3cc21]

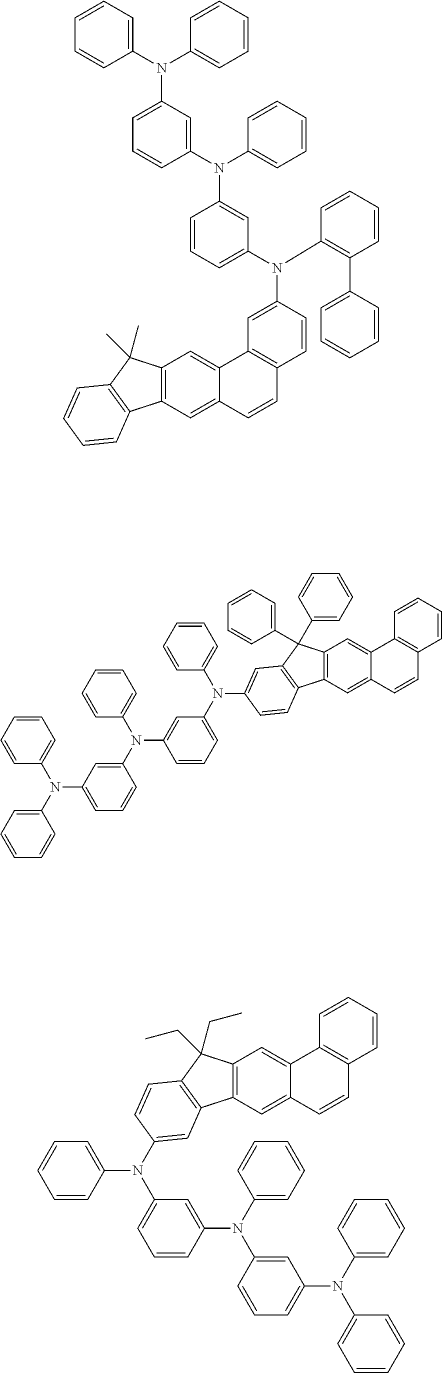 [SMILES: CC1(C)c2ccccc2-c2cc3ccc4ccc(N(c5cccc(N(c6ccccc6)c6cccc(N(c7ccccc7)c7ccccc7)c6)c5)c5ccccc5-c5ccccc5)cc4c3cc21 ; c1ccc(N(c2ccccc2)c2cccc(N(c3ccccc3)c3cccc(N(c4ccccc4)c4ccc5c(c4)C(c4ccccc4)(c4ccccc4)c4cc6c(ccc7ccccc76)cc4-5)c3)c2)cc1 ; CCC1(CC)c2ccc(N(c3ccccc3)c3cccc(N(c4ccccc4)c4cccc(N(c5ccccc5)c5ccccc5)c4)c3)cc2-c2cc3ccc4ccccc4c3cc21]

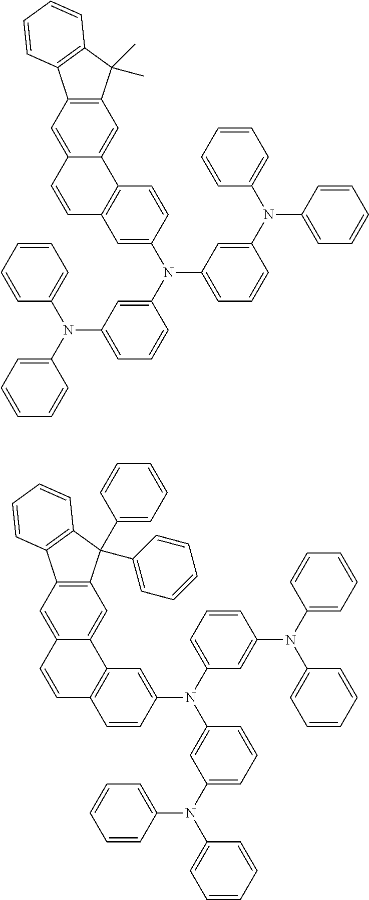 [SMILES: CC1(C)c2ccccc2-c2cc3ccc4cc(N(c5cccc(N(c6ccccc6)c6ccccc6)c5)c5cccc(N(c6ccccc6)c6ccccc6)c5)ccc4c3cc21 ; c1ccc(N(c2ccccc2)c2cccc(N(c3cccc(N(c4ccccc4)c4ccccc4)c3)c3ccc4ccc5cc6c(cc5c4c3)C(c3ccccc3)(c3ccccc3)c3ccccc3-6)c2)cc1]

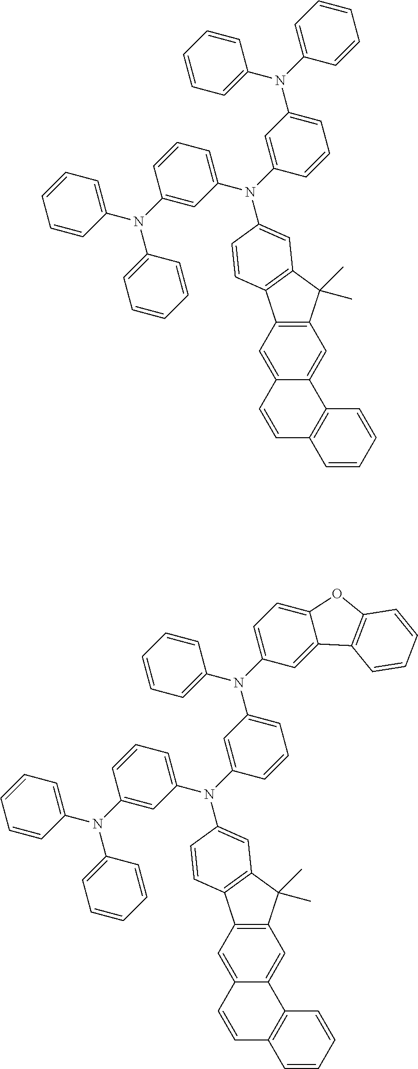 [SMILES: CC1(C)c2cc(N(c3cccc(N(c4ccccc4)c4ccccc4)c3)c3cccc(N(c4ccccc4)c4ccccc4)c3)ccc2-c2cc3ccc4ccccc4c3cc21 ; CC1(C)c2cc(N(c3cccc(N(c4ccccc4)c4ccccc4)c3)c3cccc(N(c4ccccc4)c4ccc5oc6ccccc6c5c4)c3)ccc2-c2cc3ccc4ccccc4c3cc21]

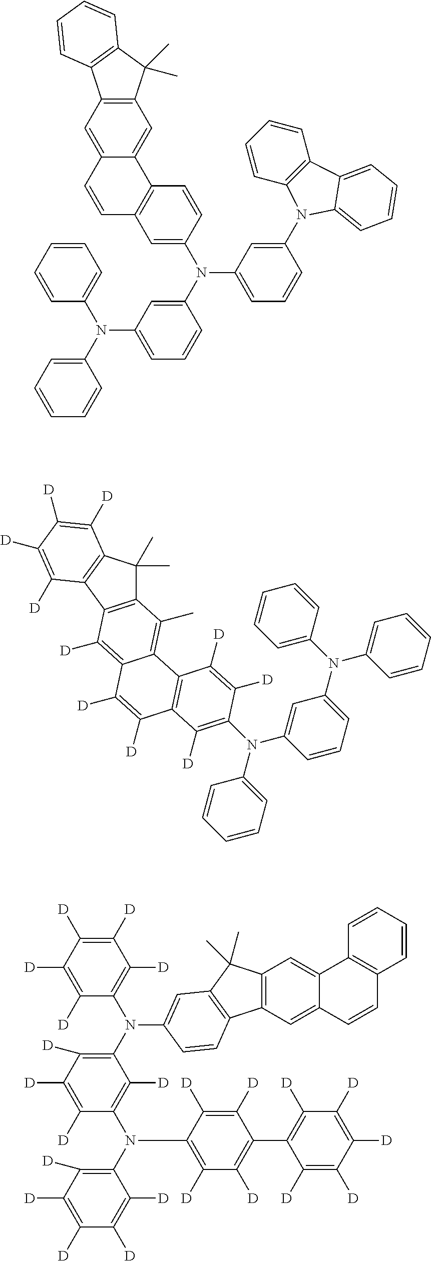 [SMILES: CC1(C)c2ccccc2-c2cc3ccc4cc(N(c5cccc(N(c6ccccc6)c6ccccc6)c5)c5cccc(-n6c7ccccc7c7ccccc76)c5)ccc4c3cc21 ; [2H]c1c([2H])c([2H])c2c(c1[2H])-c1c(c(C)c3c(c1[2H])c([2H])c([2H])c1c([2H])c(N(c4ccccc4)c4cccc(N(c5ccccc5)c5ccccc5)c4)c([2H])c([2H])c13)C2(C)C ; [2H]c1c([2H])c([2H])c(-c2c([2H])c([2H])c(N(c3c([2H])c([2H])c([2H])c([2H])c3[2H])c3c([2H])c([2H])c([2H])c(N(c4ccc5c(c4)C(C)(C)c4cc6c(ccc7ccccc76)cc4-5)c4c([2H])c([2H])c([2H])c([2H])c4[2H])c3[2H])c([2H])c2[2H])c([2H])c1[2H]]

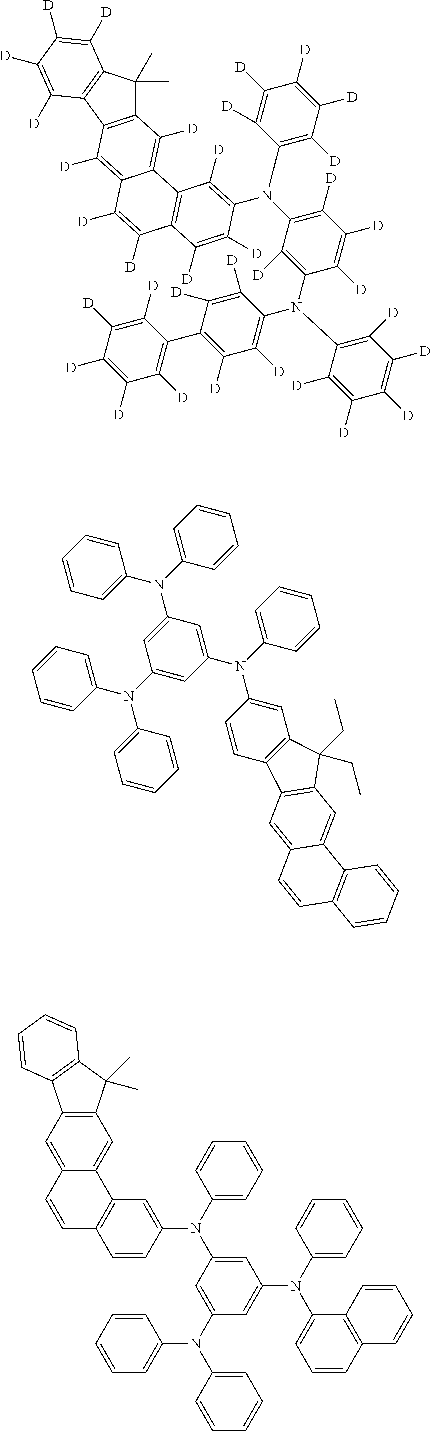 [SMILES: [2H]c1c([2H])c([2H])c(-c2c([2H])c([2H])c(N(c3c([2H])c([2H])c([2H])c([2H])c3[2H])c3c([2H])c([2H])c([2H])c(N(c4c([2H])c([2H])c([2H])c([2H])c4[2H])c4c([2H])c([2H])c5c([2H])c([2H])c6c([2H])c7c(c([2H])c6c5c4[2H])C(C)(C)c4c([2H])c([2H])c([2H])c([2H])c4-7)c3[2H])c([2H])c2[2H])c([2H])c1[2H] ; CCC1(CC)c2cc(N(c3ccccc3)c3cc(N(c4ccccc4)c4ccccc4)cc(N(c4ccccc4)c4ccccc4)c3)ccc2-c2cc3ccc4ccccc4c3cc21 ; CC1(C)c2ccccc2-c2cc3ccc4ccc(N(c5ccccc5)c5cc(N(c6ccccc6)c6ccccc6)cc(N(c6ccccc6)c6cccc7ccccc67)c5)cc4c3cc21]

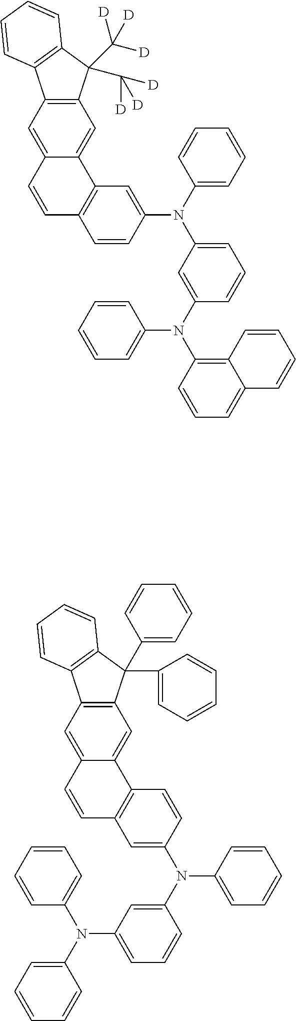 [SMILES: [2H]C([2H])([2H])C1(C([2H])([2H])[2H])c2ccccc2-c2cc3ccc4ccc(N(c5ccccc5)c5cccc(N(c6ccccc6)c6cccc7ccccc67)c5)cc4c3cc21 ; c1ccc(N(c2ccccc2)c2cccc(N(c3ccccc3)c3ccc4c(ccc5cc6c(cc54)C(c4ccccc4)(c4ccccc4)c4ccccc4-6)c3)c2)cc1]

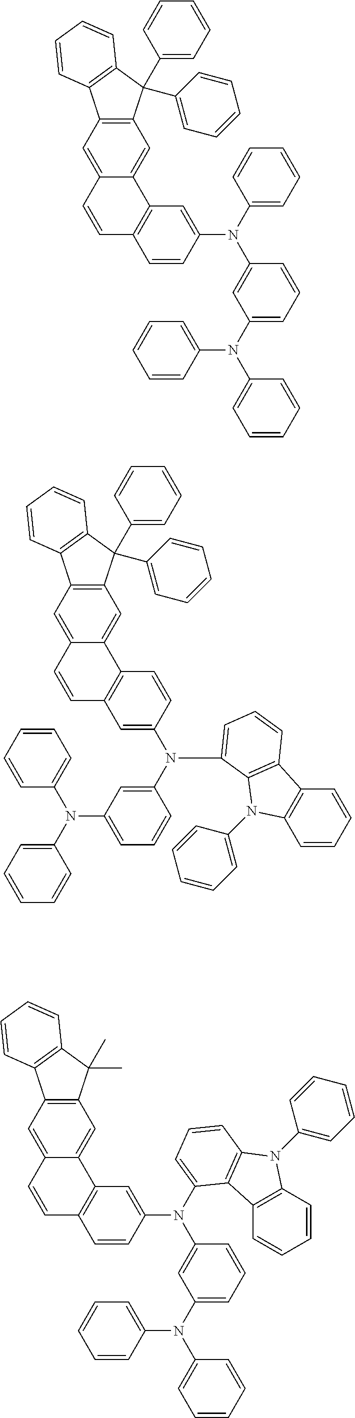 [SMILES: c1ccc(N(c2ccccc2)c2cccc(N(c3ccccc3)c3ccc4ccc5cc6c(cc5c4c3)C(c3ccccc3)(c3ccccc3)c3ccccc3-6)c2)cc1 ; c1ccc(N(c2ccccc2)c2cccc(N(c3ccc4c(ccc5cc6c(cc54)C(c4ccccc4)(c4ccccc4)c4ccccc4-6)c3)c3cccc4c5ccccc5n(-c5ccccc5)c34)c2)cc1 ; CC1(C)c2ccccc2-c2cc3ccc4ccc(N(c5cccc(N(c6ccccc6)c6ccccc6)c5)c5cccc6c5c5ccccc5n6-c5ccccc5)cc4c3cc21]

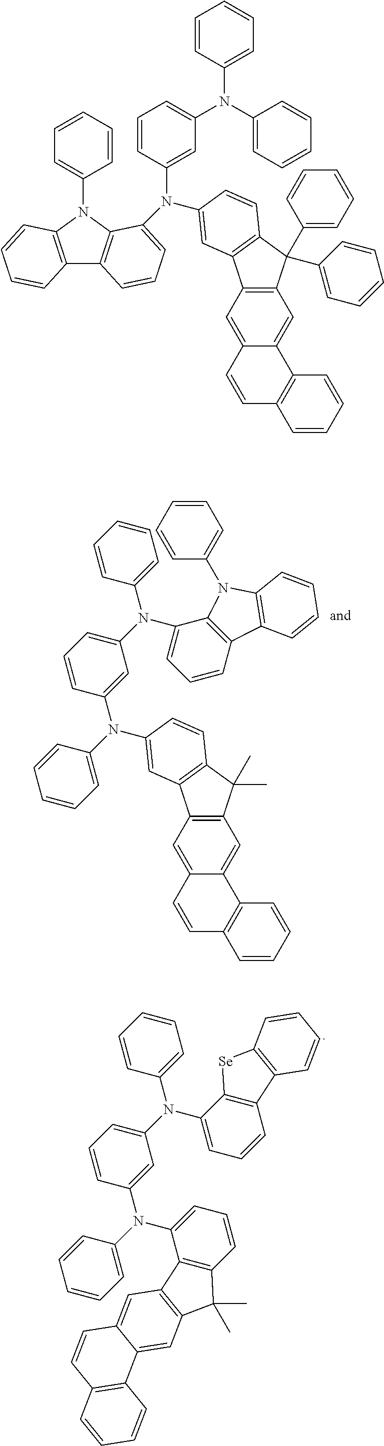 [SMILES: c1ccc(N(c2ccccc2)c2cccc(N(c3ccc4c(c3)-c3cc5ccc6ccccc6c5cc3C4(c3ccccc3)c3ccccc3)c3cccc4c5ccccc5n(-c5ccccc5)c34)c2)cc1 ; CC1(C)c2ccc(N(c3ccccc3)c3cccc(N(c4ccccc4)c4cccc5c6ccccc6n(-c6ccccc6)c45)c3)cc2-c2cc3ccc4ccccc4c3cc21 ; CC1(C)c2cc3c(ccc4ccccc43)cc2-c2c(N(c3ccccc3)c3cccc(N(c4ccccc4)c4cccc5c4[se]c4ccccc45)c3)cccc21]

7. An organic electroluminescent material comprising the organic electroluminescent compound according to claim 1.

8. The organic electroluminescent material according to claim 7, which further comprises at least one compound represented by any one of the following formulas 11 to 15: 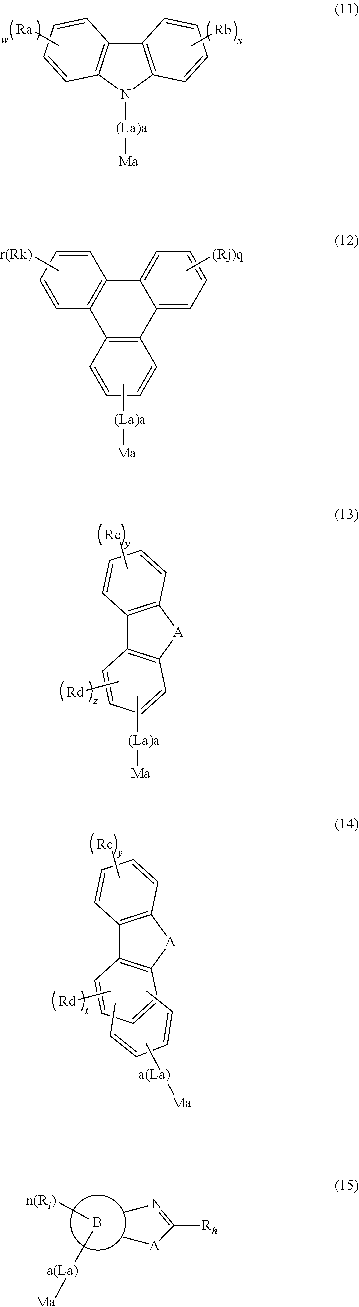
in formulas 11 to 15,
Ma represents a substituted or unsubstituted (C6-C30)aryl, a substituted or unsubstituted mono- or di- (C6-C30)arylamino, a substituted or unsubstituted mono- or di- (3- to 30-membered)heteroarylamino, a substituted or unsubstituted (C6-C30)aryl(3- to 30-membered)heteroarylamino, or a substituted or unsubstituted (3- to 30-membered)heteroaryl;
La represents a single bond, a substituted or unsubstituted (C6-C30)arylene, or a substituted or unsubstituted (3- to 30-membered)heteroarylene;
A represents S, O, N(Re), C(Rf)(Rg), Te, or Se;
ring B represents a naphthalene ring or a phenanthrene ring;
Ra to Rd and Rh to Rk, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di- (C1-C30)alkylamino, a substituted or unsubstituted (C1-C30)alkyl(C6-C30)arylamino, or a substituted or unsubstituted mono- or di-(C6-C30)arylamino; or may be linked to an adjacent substituent(s) to form a ring(s);
Re to Rg, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30)arylamino; and Rf and Rg may be linked to each other to form a ring(s);
w to y, q, and r, each independently, represent an integer of 1 to 4; z represents an integer of 1 to 3; a and t, each independently, represent an integer of 1 or 2; and n represents an integer of 1 to 9; and each of Ra to each of Rd, each of R₁ to each of Rk, and each of La may be the same as or different from each other; and
the heteroaryl(ene) contains at least one heteroatom selected from B, N, O, S, Si, P, Te, and Se.

9. The organic electroluminescent material according to claim 8, wherein the compound represented by formula 11 is selected from the group consisting of the following compounds:

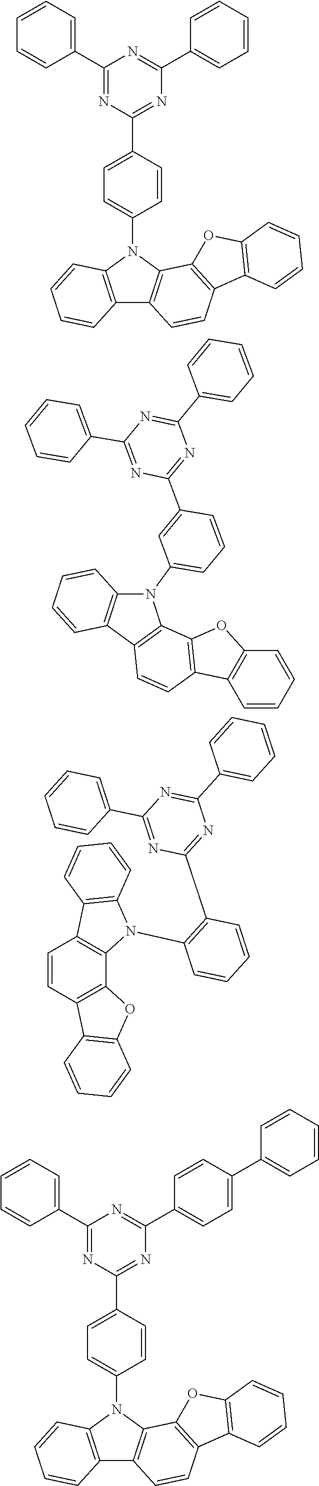

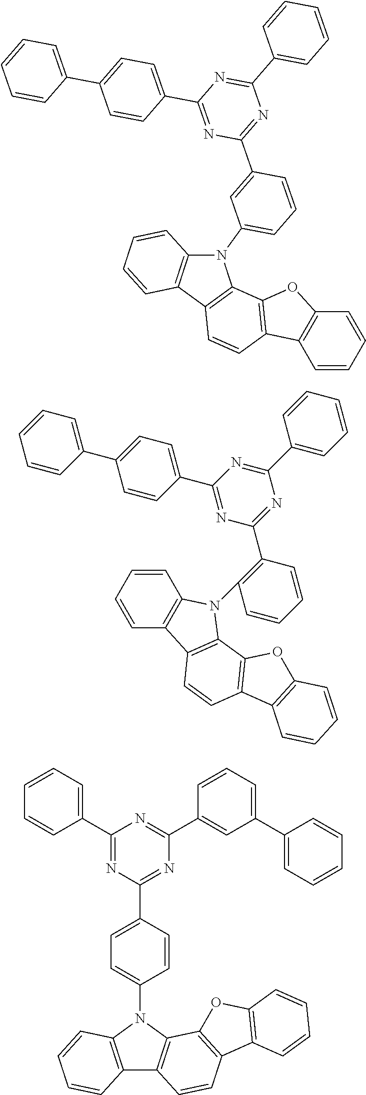

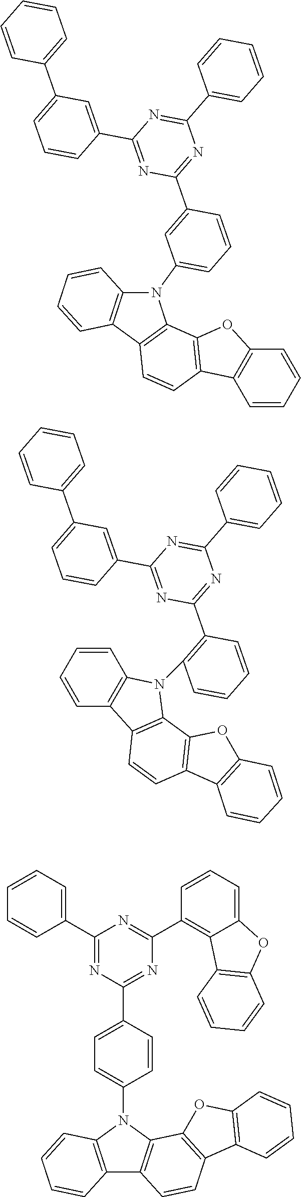 [SMILES: c1ccc(-c2cccc(-c3nc(-c4ccccc4)nc(-c4cccc(-n5c6ccccc6c6ccc7c8ccccc8oc7c65)c4)n3)c2)cc1 ; c1ccc(-c2cccc(-c3nc(-c4ccccc4)nc(-c4ccccc4-n4c5ccccc5c5ccc6c7ccccc7oc6c54)n3)c2)cc1 ; c1ccc(-c2nc(-c3ccc(-n4c5ccccc5c5ccc6c7ccccc7oc6c54)cc3)nc(-c3cccc4oc5ccccc5c34)n2)cc1]

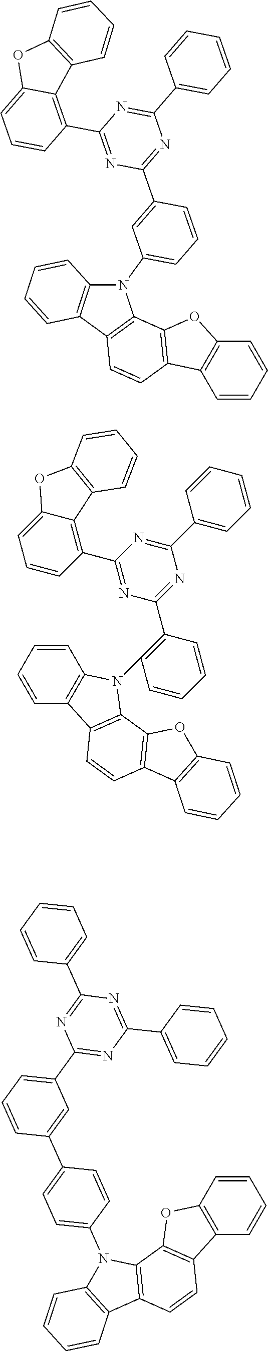 [SMILES: c1ccc(-c2nc(-c3cccc(-n4c5ccccc5c5ccc6c7ccccc7oc6c54)c3)nc(-c3cccc4oc5ccccc5c34)n2)cc1 ; c1ccc(-c2nc(-c3ccccc3-n3c4ccccc4c4ccc5c6ccccc6oc5c43)nc(-c3cccc4oc5ccccc5c34)n2)cc1 ; c1ccc(-c2nc(-c3ccccc3)nc(-c3cccc(-c4ccc(-n5c6ccccc6c6ccc7c8ccccc8oc7c65)cc4)c3)n2)cc1]

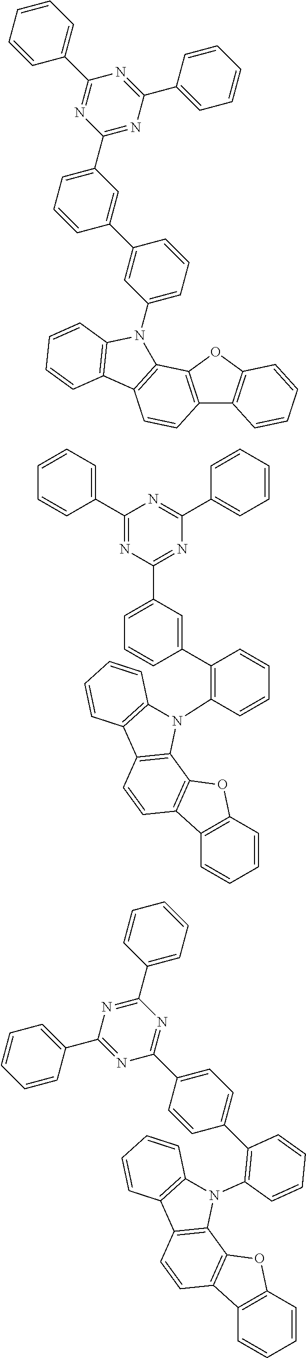

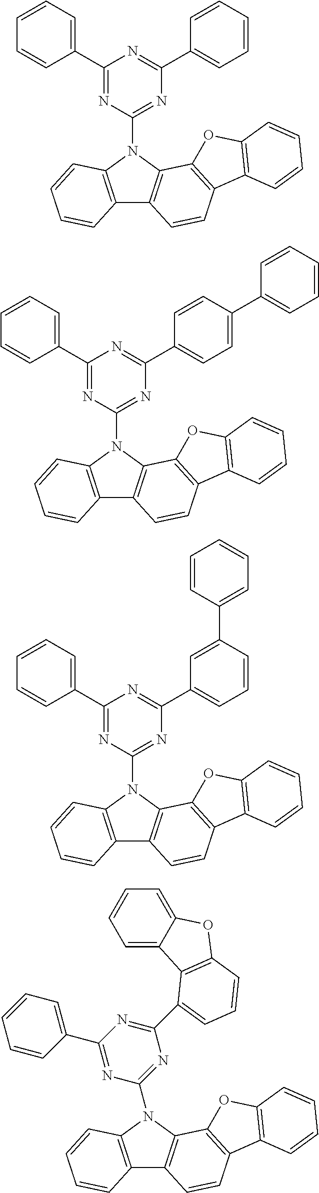

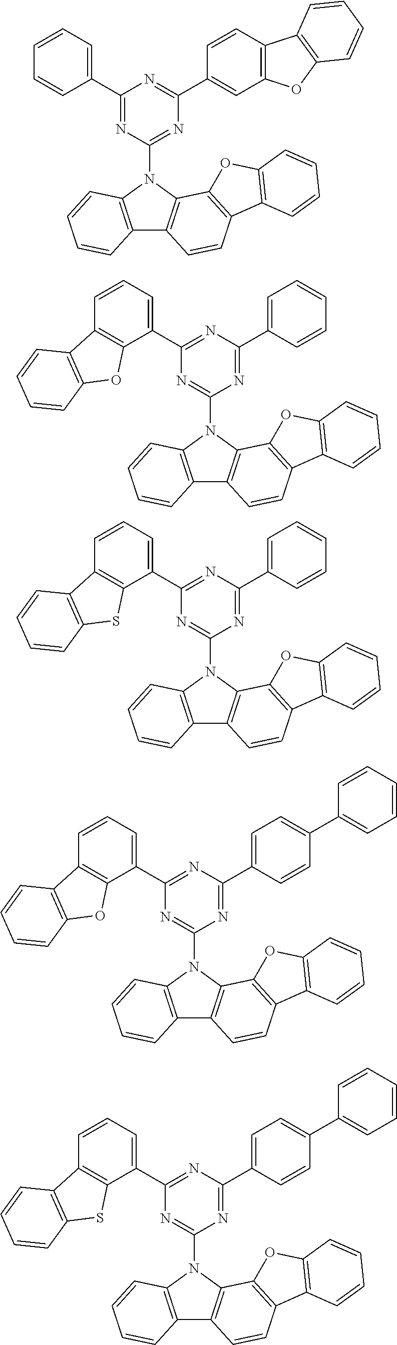

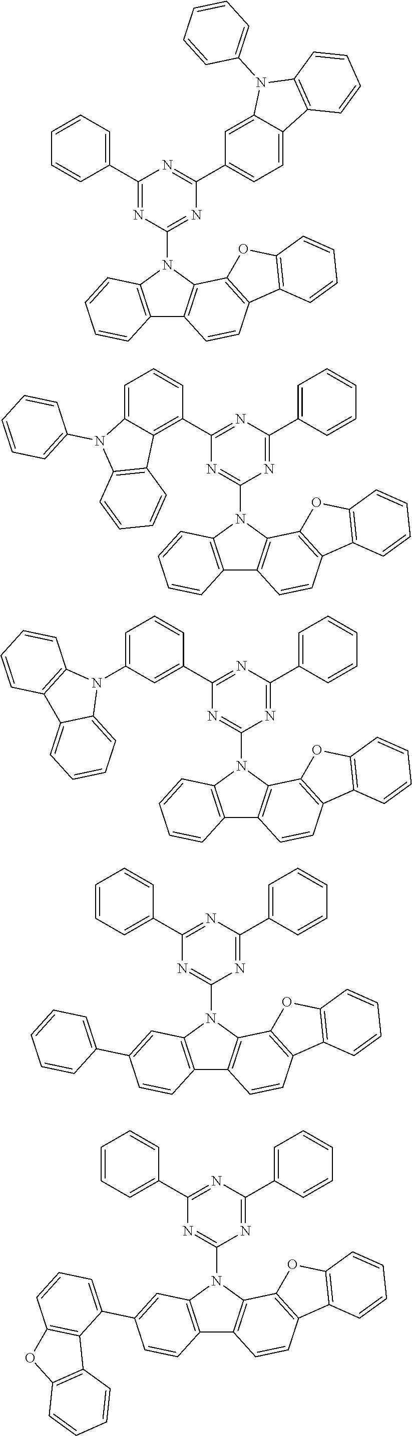

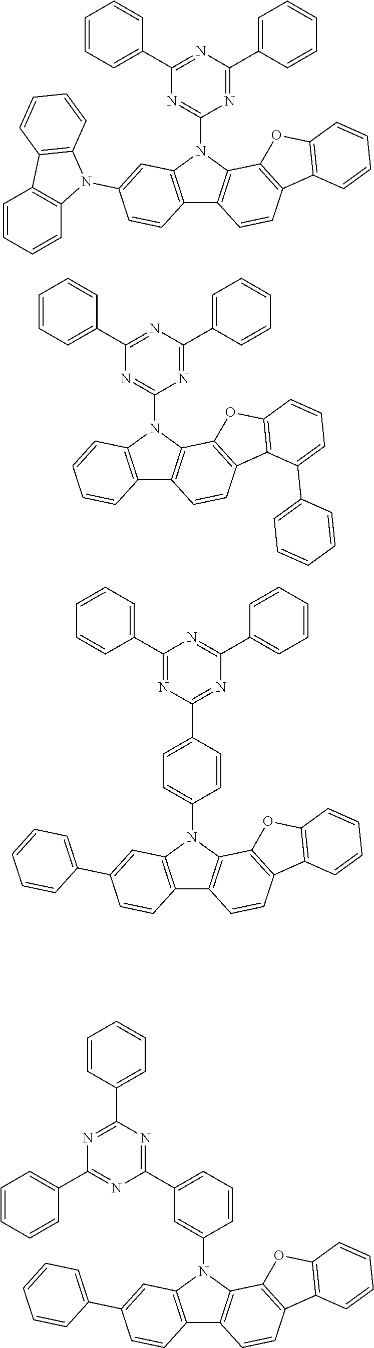

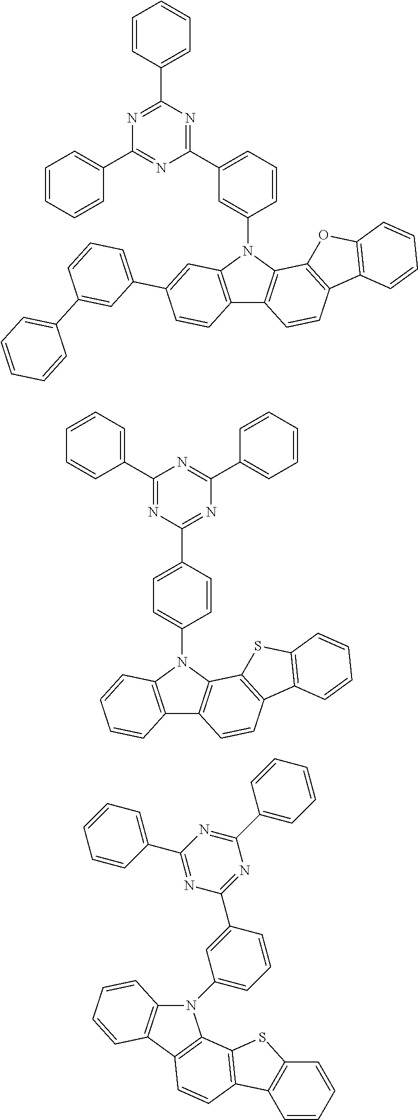

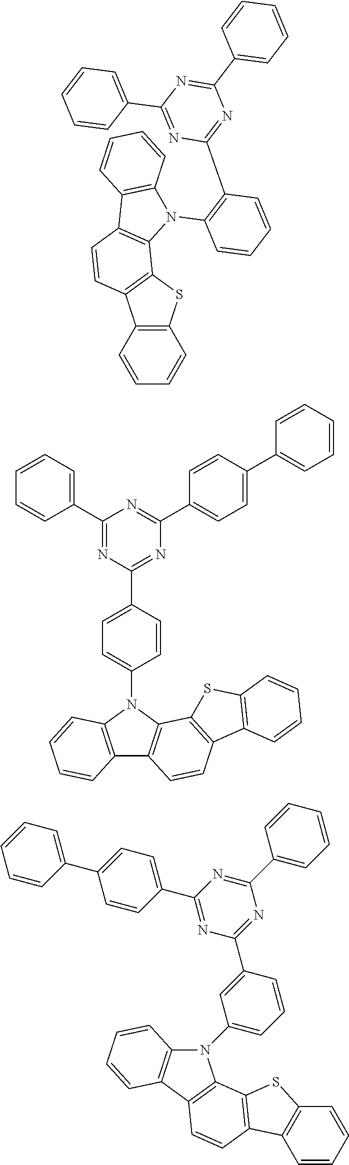 [SMILES: c1ccc(-c2nc(-c3ccccc3)nc(-c3ccccc3-n3c4ccccc4c4ccc5c6ccccc6sc5c43)n2)cc1 ; c1ccc(-c2ccc(-c3nc(-c4ccccc4)nc(-c4ccc(-n5c6ccccc6c6ccc7c8ccccc8sc7c65)cc4)n3)cc2)cc1 ; c1ccc(-c2ccc(-c3nc(-c4ccccc4)nc(-c4cccc(-n5c6ccccc6c6ccc7c8ccccc8sc7c65)c4)n3)cc2)cc1]

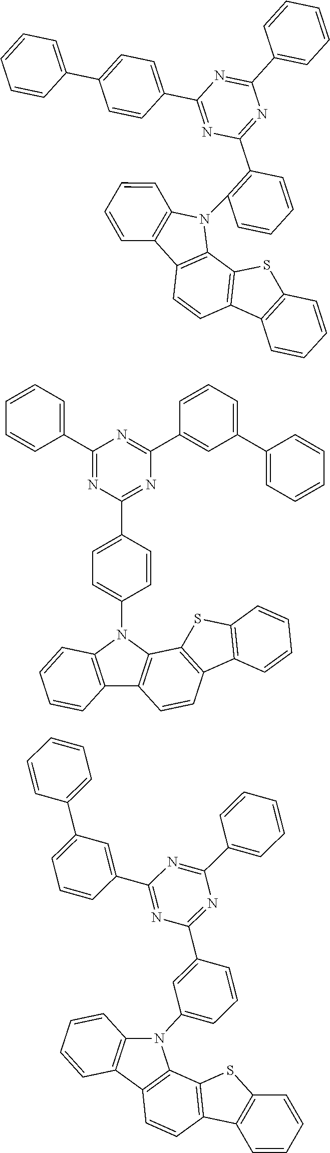

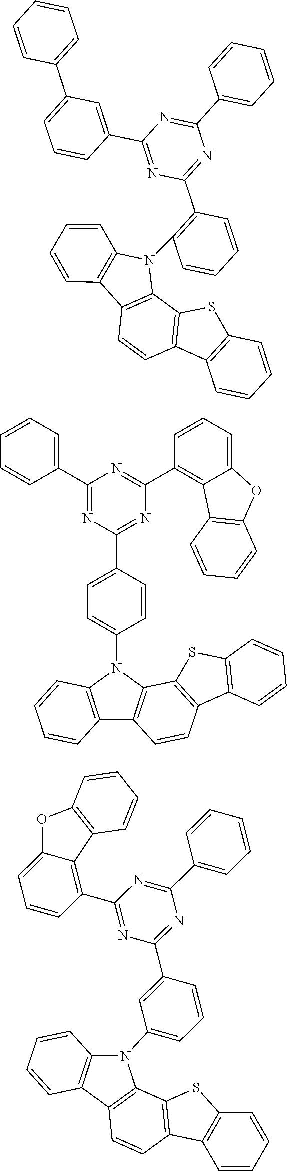

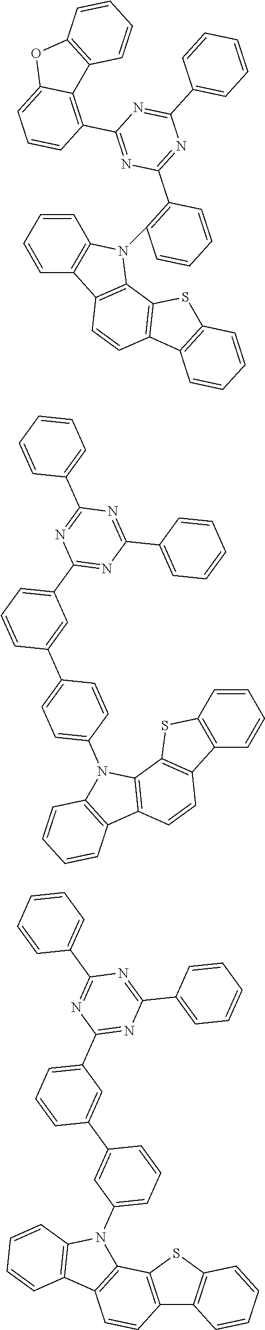

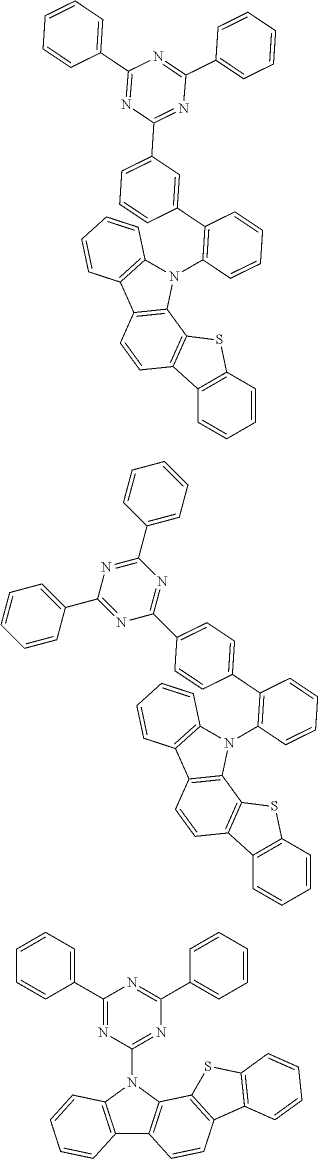 [SMILES: c1ccc(-c2nc(-c3ccccc3)nc(-c3cccc(-c4ccccc4-n4c5ccccc5c5ccc6c7ccccc7sc6c54)c3)n2)cc1 ; c1ccc(-c2nc(-c3ccccc3)nc(-c3ccc(-c4ccccc4-n4c5ccccc5c5ccc6c7ccccc7sc6c54)cc3)n2)cc1 ; c1ccc(-c2nc(-c3ccccc3)nc(-n3c4ccccc4c4ccc5c6ccccc6sc5c43)n2)cc1]

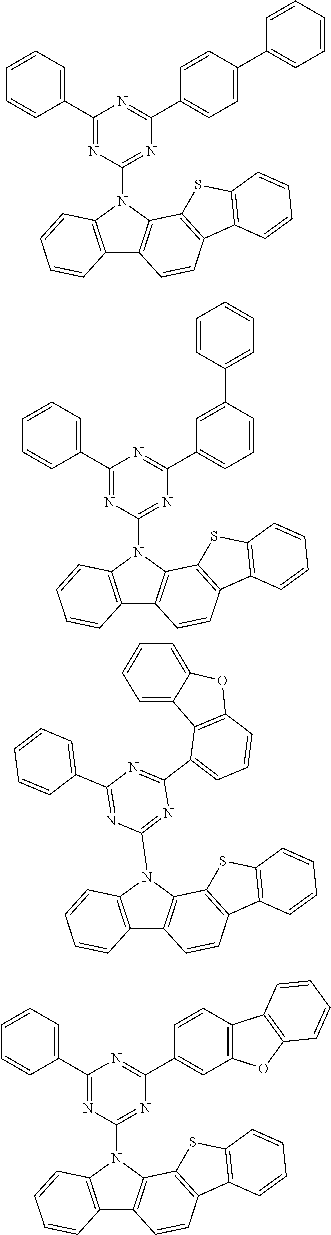

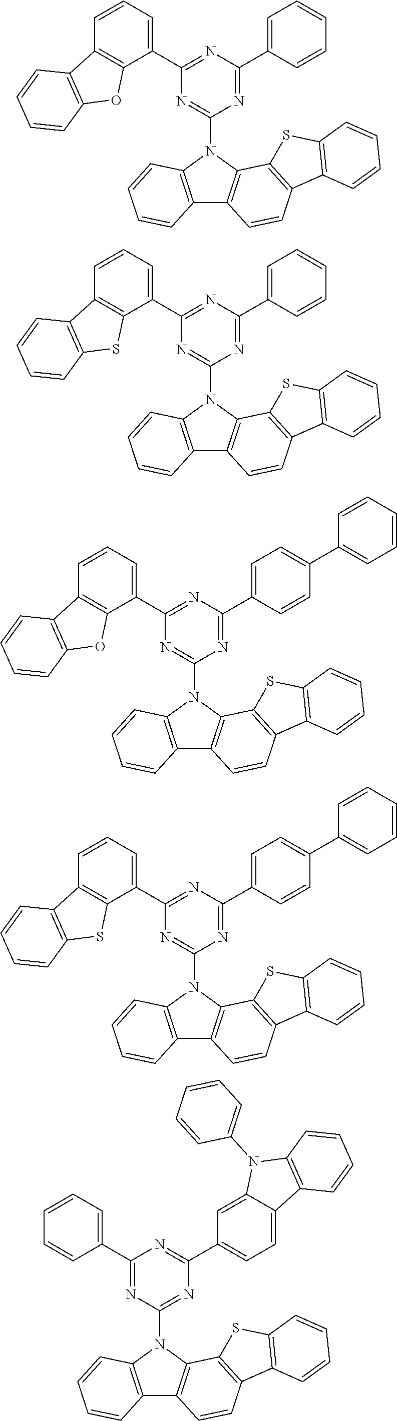

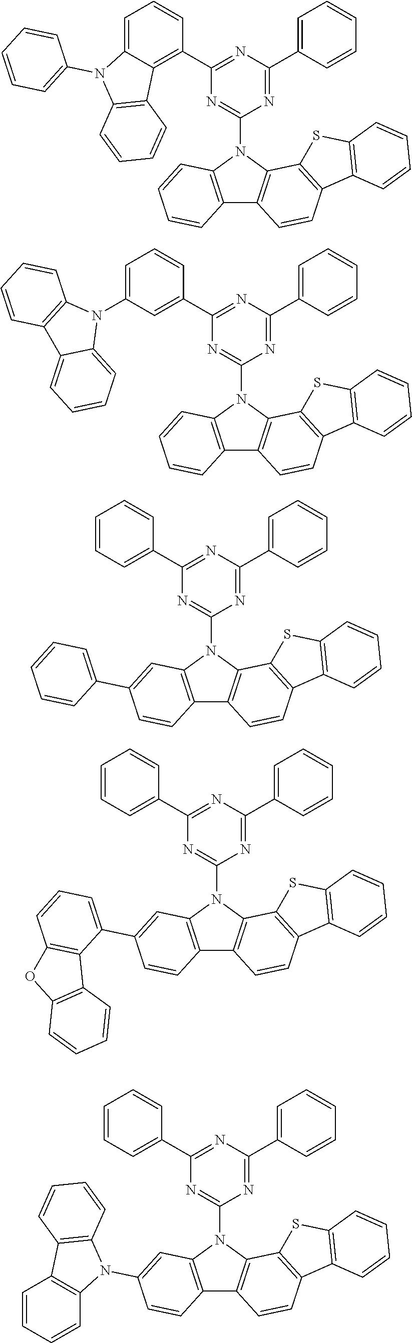

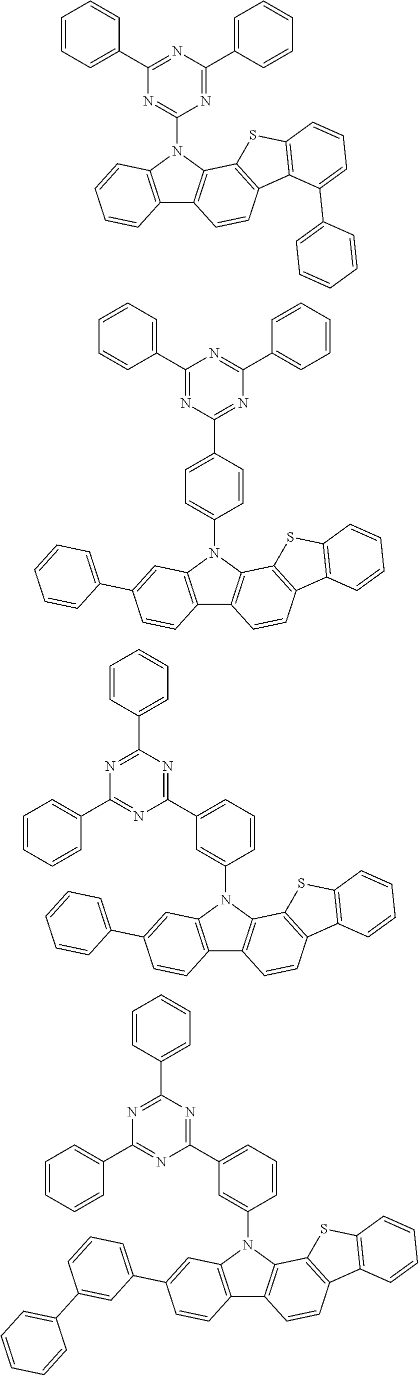

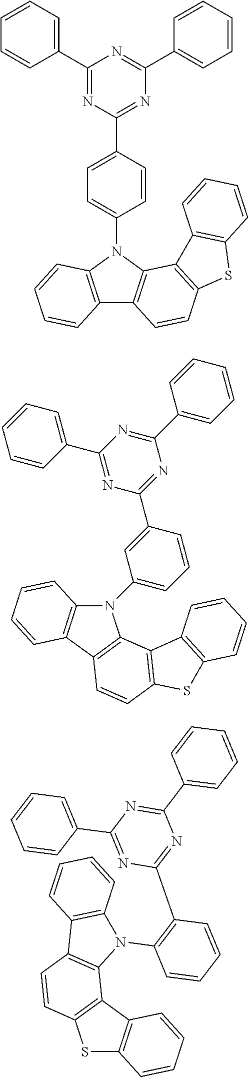

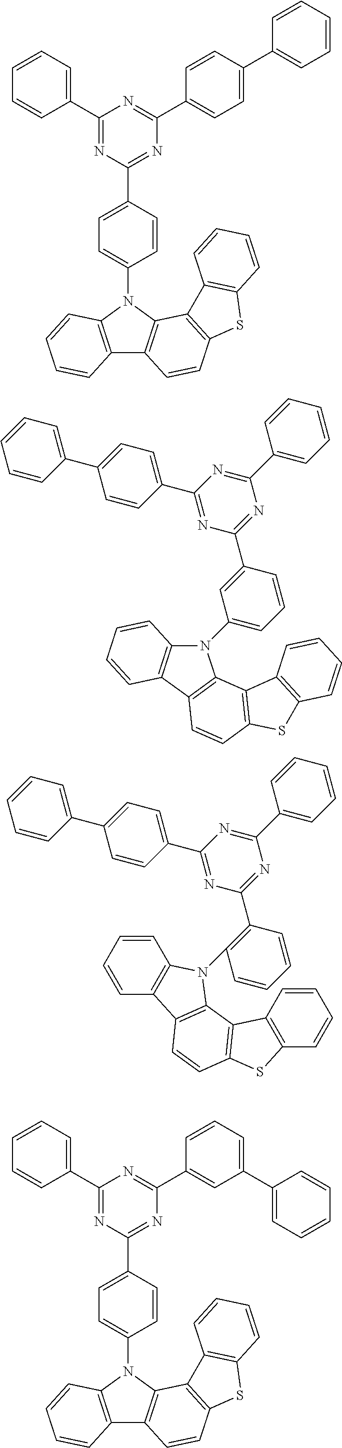

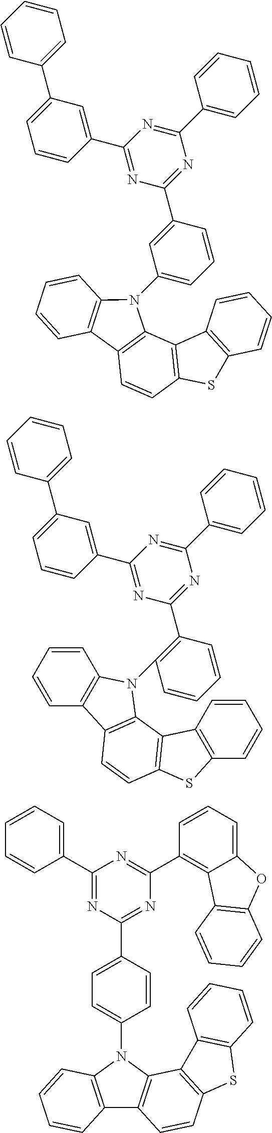

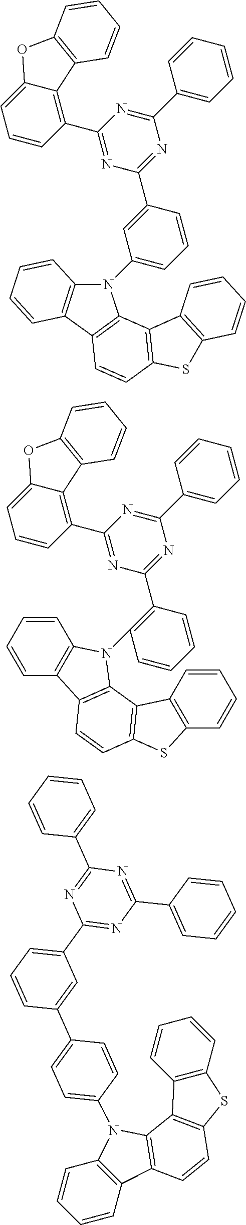

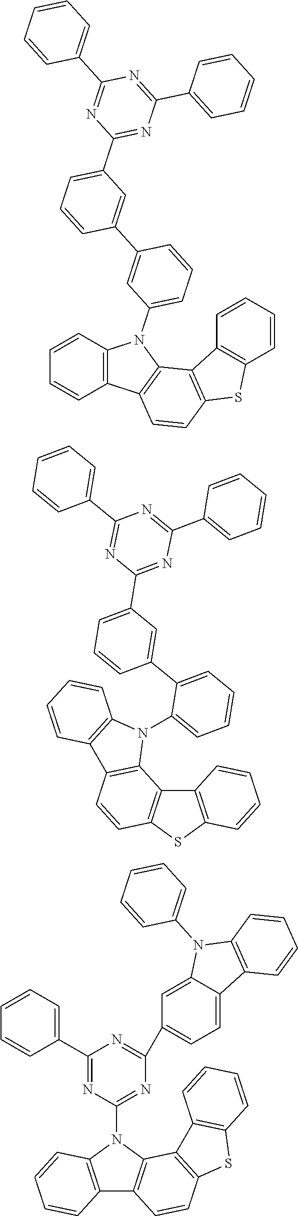

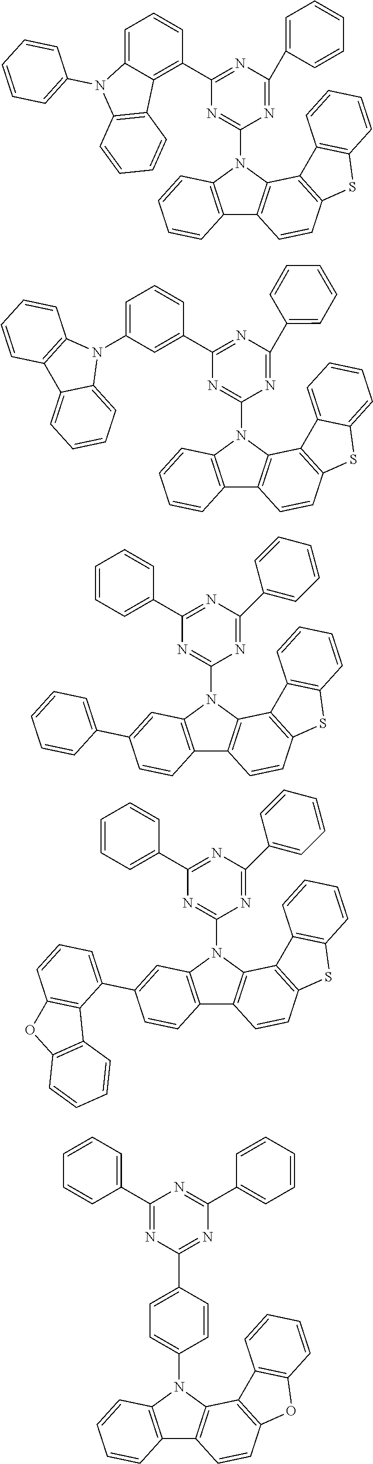

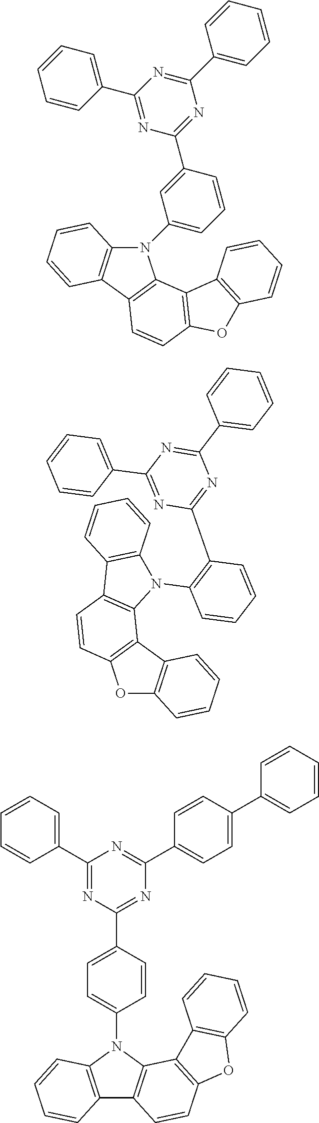

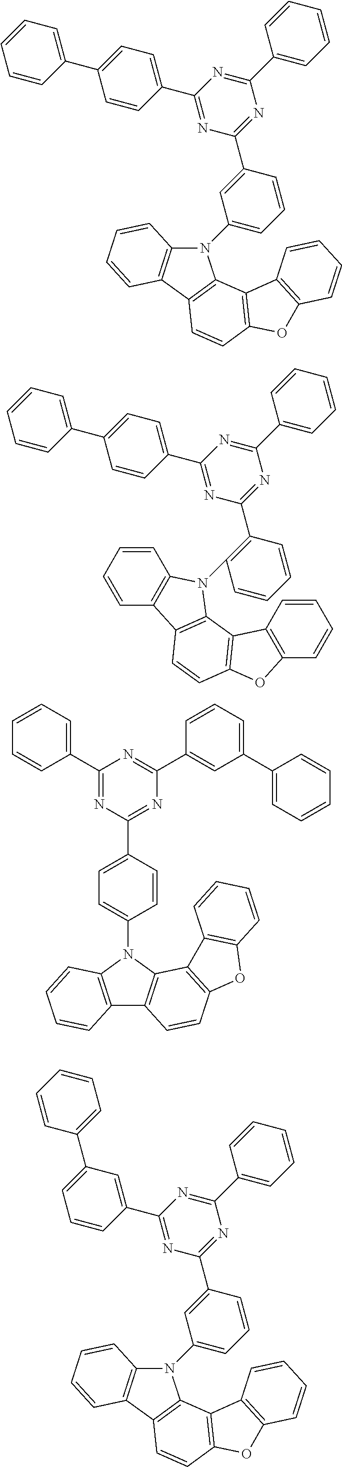

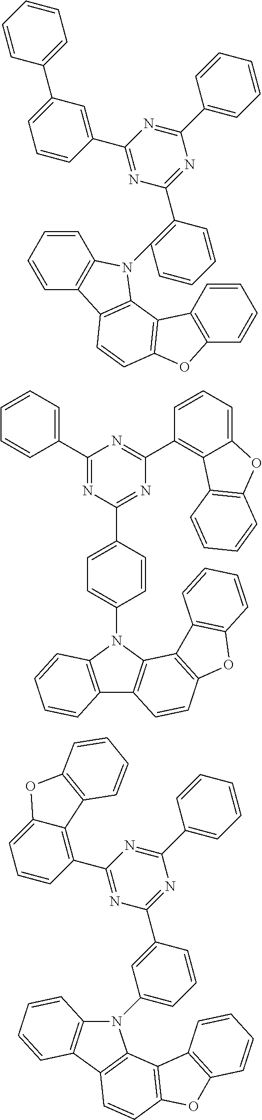

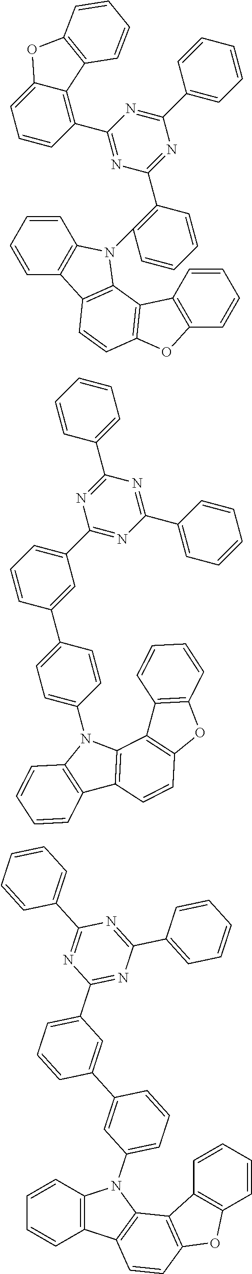

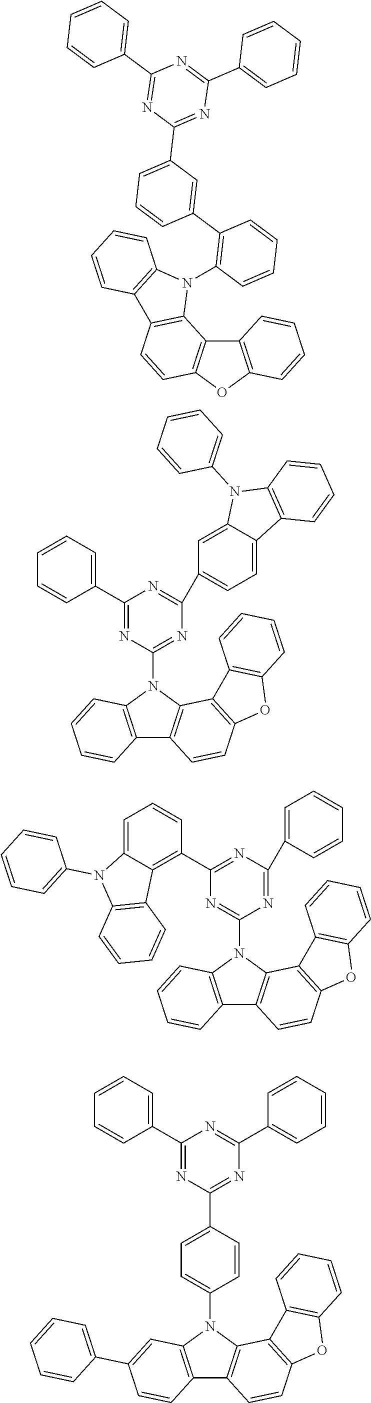

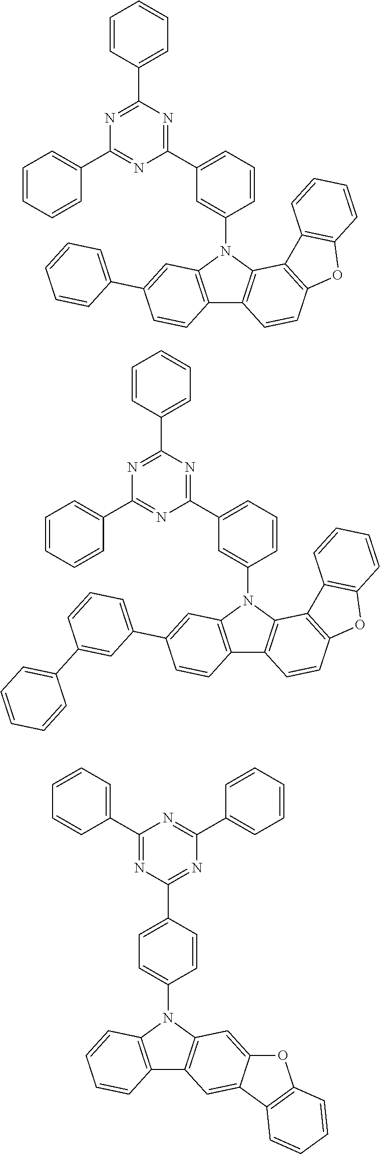

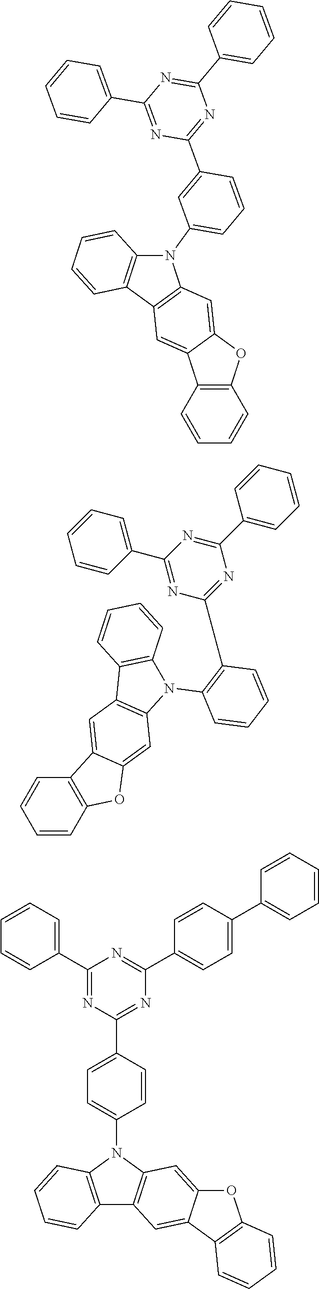

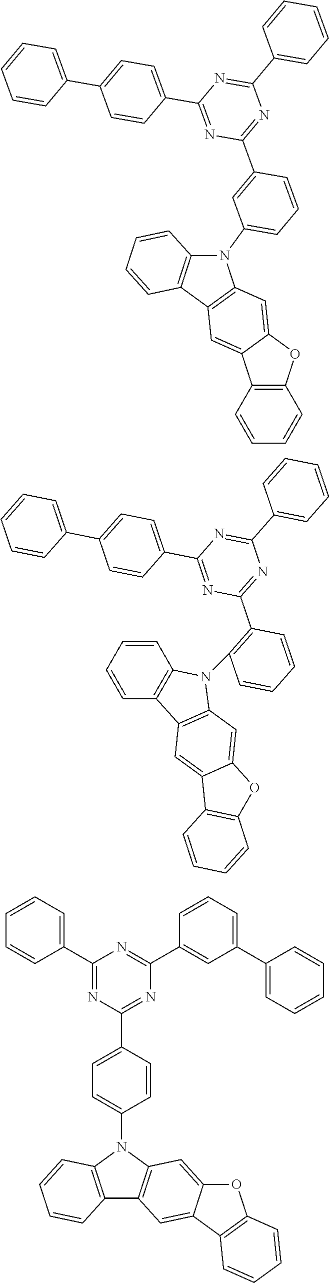

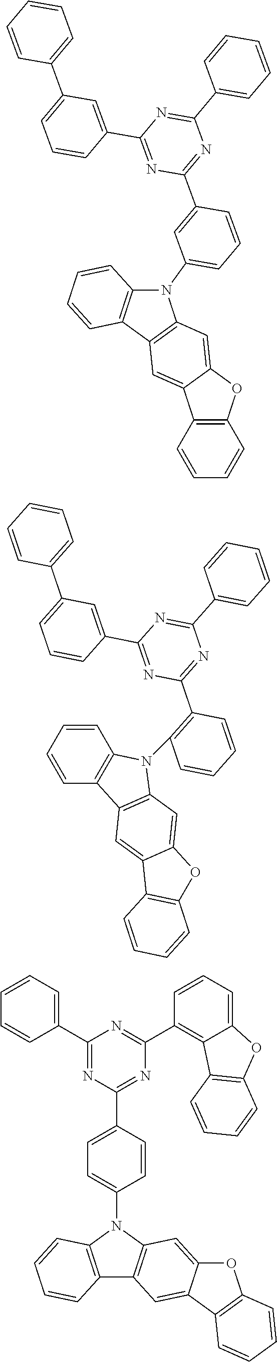

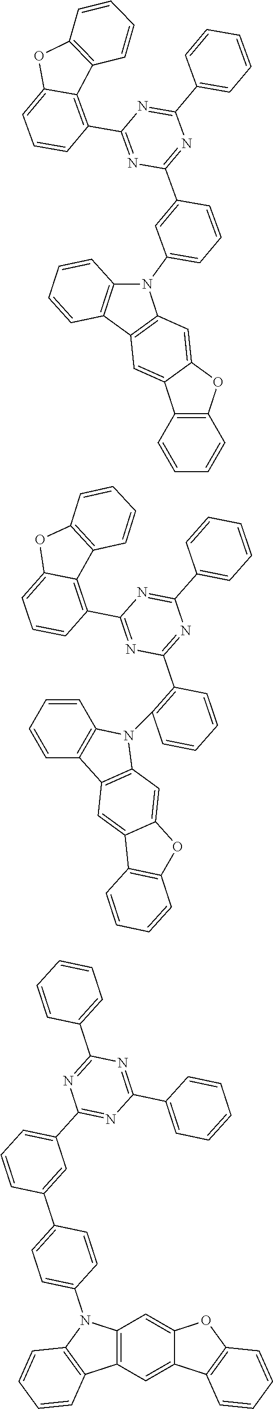

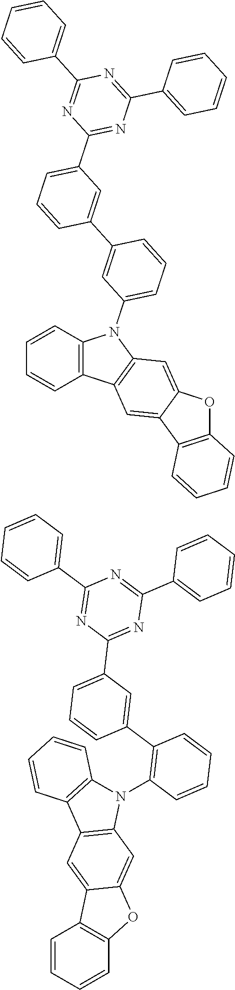

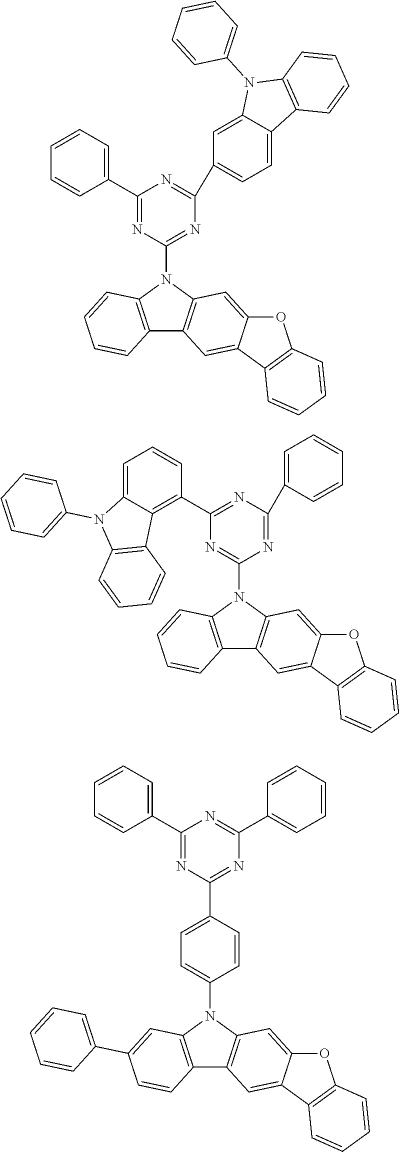

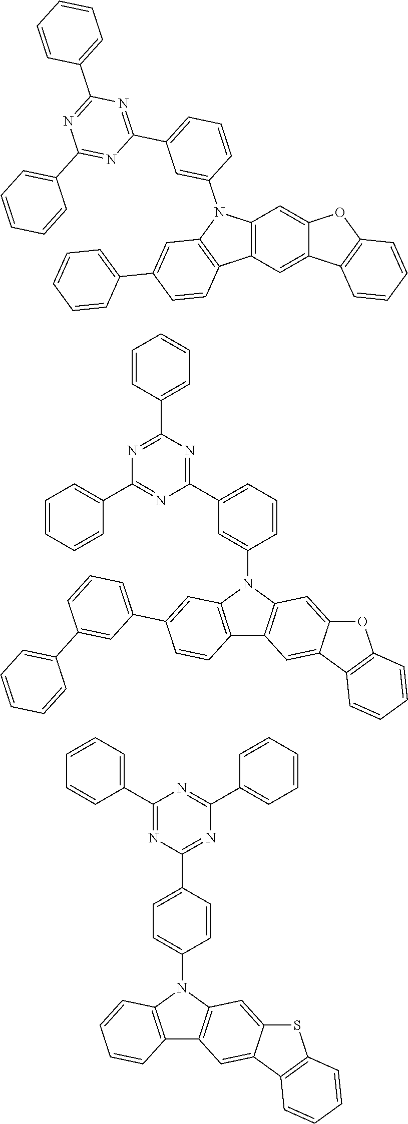

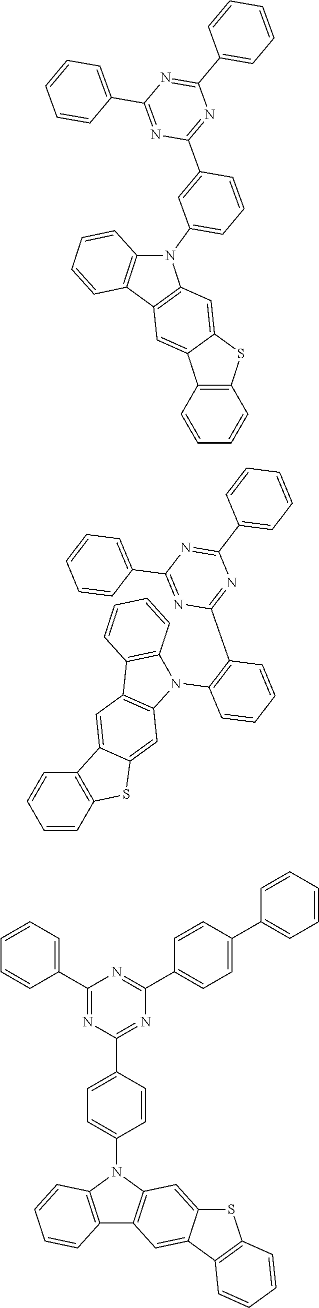

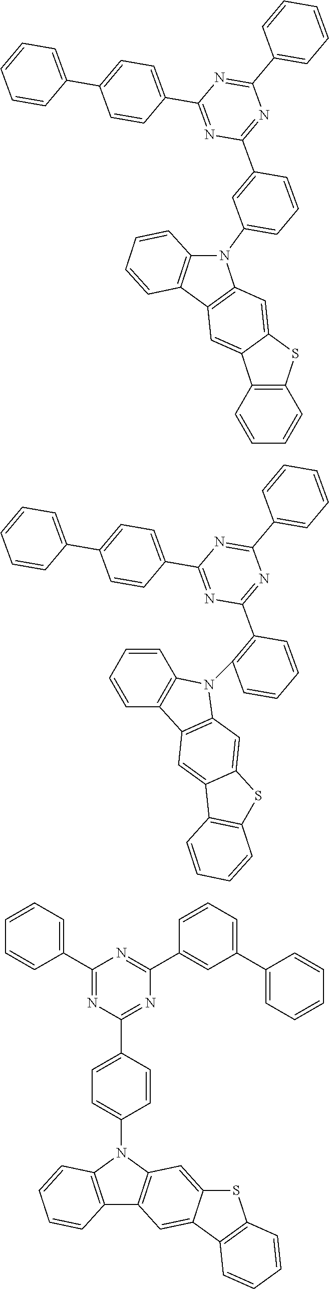

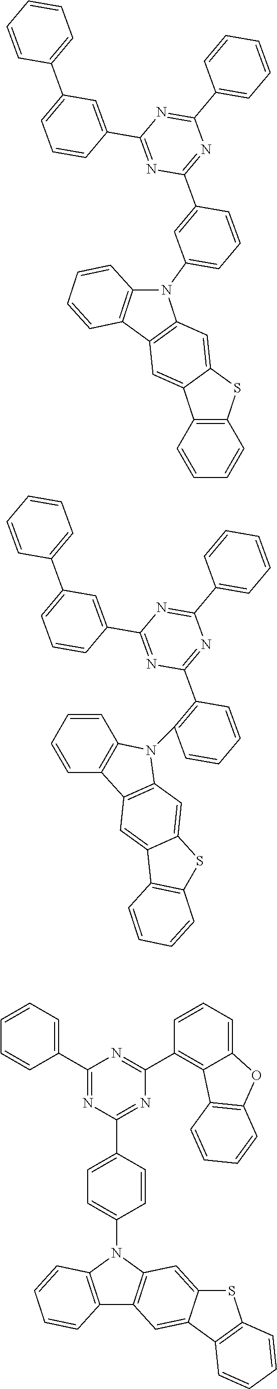 [SMILES: c1ccc(-c2cccc(-c3nc(-c4ccccc4)nc(-c4cccc(-n5c6ccccc6c6cc7c(cc65)sc5ccccc57)c4)n3)c2)cc1 ; c1ccc(-c2cccc(-c3nc(-c4ccccc4)nc(-c4ccccc4-n4c5ccccc5c5cc6c(cc54)sc4ccccc46)n3)c2)cc1 ; c1ccc(-c2nc(-c3ccc(-n4c5ccccc5c5cc6c(cc54)sc4ccccc46)cc3)nc(-c3cccc4oc5ccccc5c34)n2)cc1]

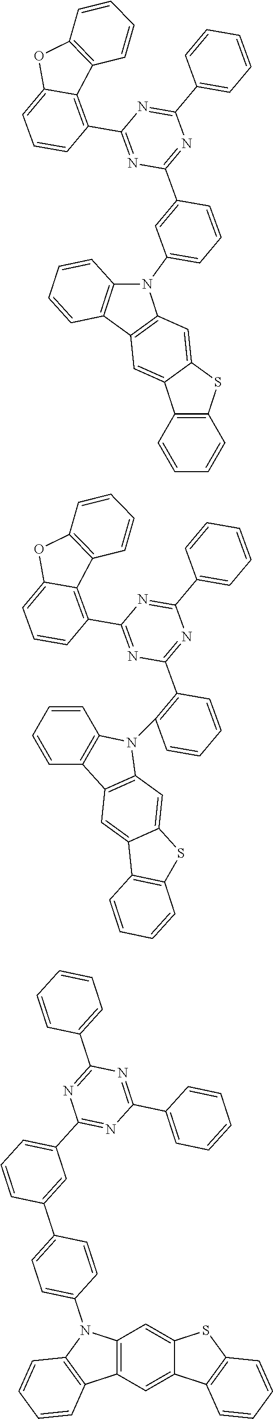 [SMILES: c1ccc(-c2nc(-c3cccc(-n4c5ccccc5c5cc6c(cc54)sc4ccccc46)c3)nc(-c3cccc4oc5ccccc5c34)n2)cc1 ; c1ccc(-c2nc(-c3ccccc3-n3c4ccccc4c4cc5c(cc43)sc3ccccc35)nc(-c3cccc4oc5ccccc5c34)n2)cc1 ; c1ccc(-c2nc(-c3ccccc3)nc(-c3cccc(-c4ccc(-n5c6ccccc6c6cc7c(cc65)sc5ccccc57)cc4)c3)n2)cc1]

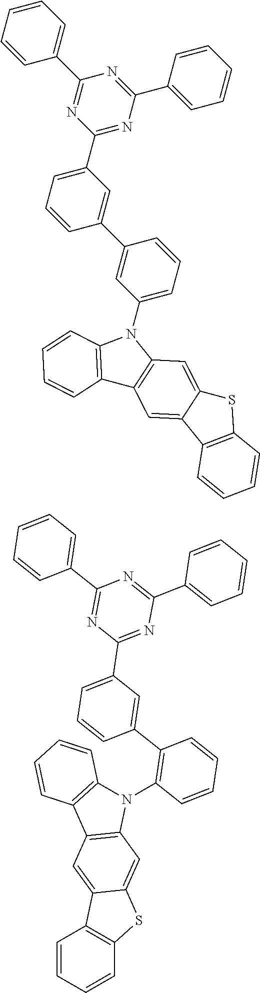

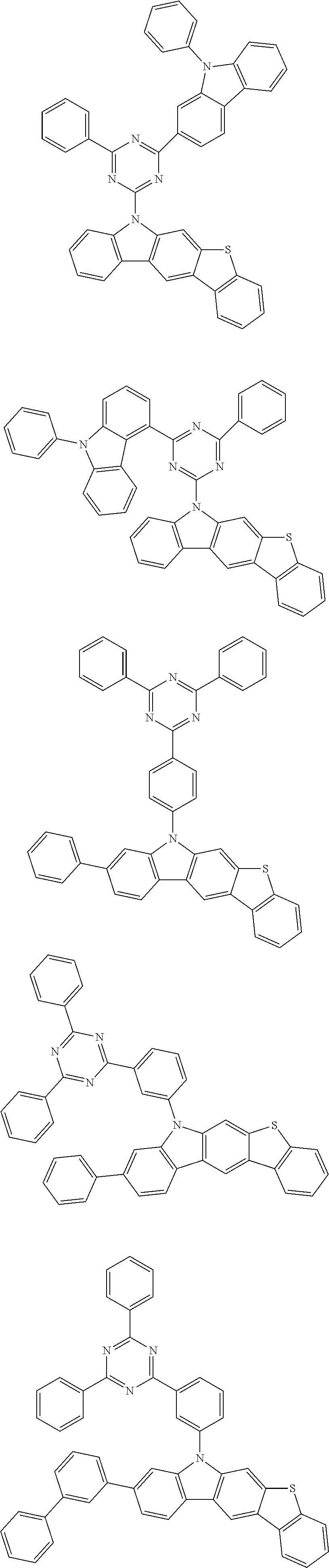 [SMILES: c1ccc(-c2nc(-c3ccc4c5ccccc5n(-c5ccccc5)c4c3)nc(-n3c4ccccc4c4cc5c(cc43)sc3ccccc35)n2)cc1 ; c1ccc(-c2nc(-c3cccc4c3c3ccccc3n4-c3ccccc3)nc(-n3c4ccccc4c4cc5c(cc43)sc3ccccc35)n2)cc1 ; c1ccc(-c2ccc3c4cc5c(cc4n(-c4ccc(-c6nc(-c7ccccc7)nc(-c7ccccc7)n6)cc4)c3c2)sc2ccccc25)cc1 ; c1ccc(-c2ccc3c4cc5c(cc4n(-c4cccc(-c6nc(-c7ccccc7)nc(-c7ccccc7)n6)c4)c3c2)sc2ccccc25)cc1 ; c1ccc(-c2cccc(-c3ccc4c5cc6c(cc5n(-c5cccc(-c7nc(-c8ccccc8)nc(-c8ccccc8)n7)c5)c4c3)sc3ccccc36)c2)cc1]

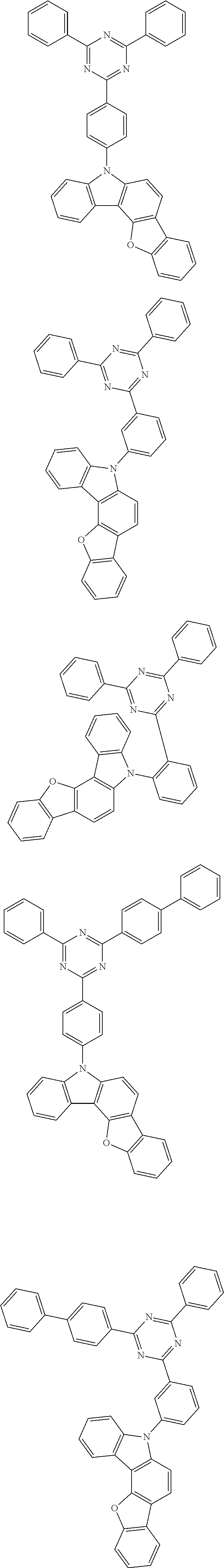

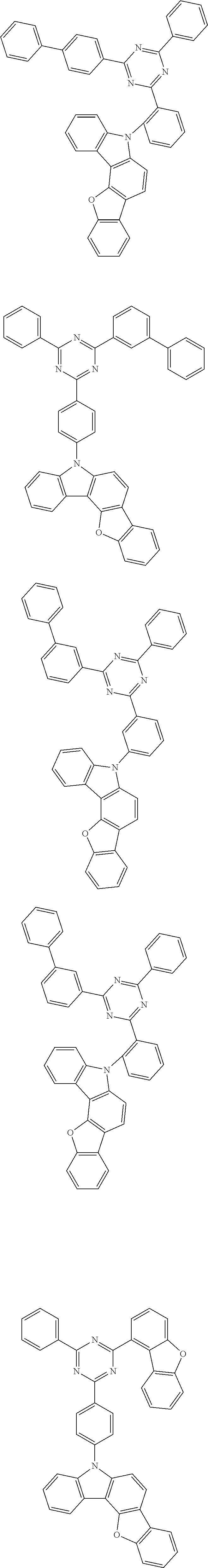 [SMILES: c1ccc(-c2ccc(-c3nc(-c4ccccc4)nc(-c4ccccc4-n4c5ccccc5c5c6oc7ccccc7c6ccc54)n3)cc2)cc1 ; c1ccc(-c2cccc(-c3nc(-c4ccccc4)nc(-c4ccc(-n5c6ccccc6c6c7oc8ccccc8c7ccc65)cc4)n3)c2)cc1 ; c1ccc(-c2cccc(-c3nc(-c4ccccc4)nc(-c4cccc(-n5c6ccccc6c6c7oc8ccccc8c7ccc65)c4)n3)c2)cc1 ; c1ccc(-c2cccc(-c3nc(-c4ccccc4)nc(-c4ccccc4-n4c5ccccc5c5c6oc7ccccc7c6ccc54)n3)c2)cc1 ; c1ccc(-c2nc(-c3ccc(-n4c5ccccc5c5c6oc7ccccc7c6ccc54)cc3)nc(-c3cccc4oc5ccccc5c34)n2)cc1]

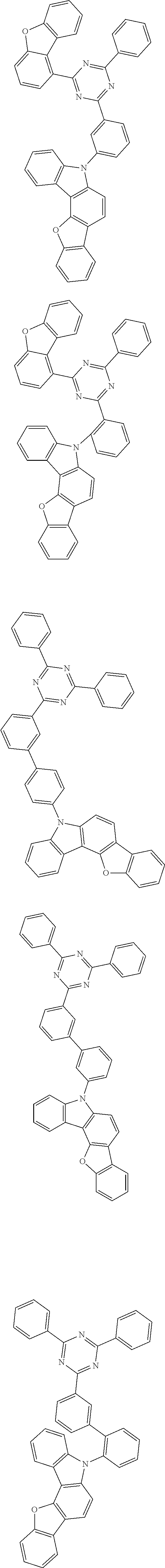

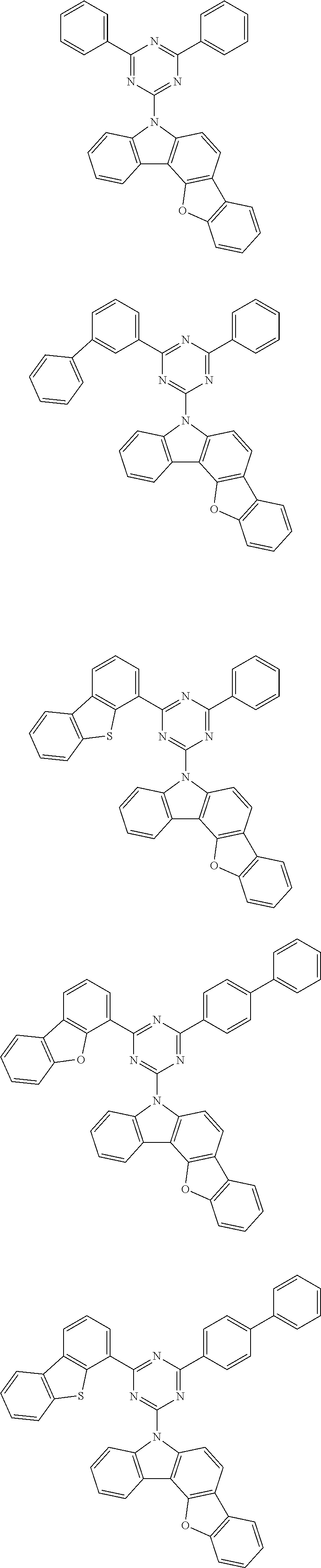 [SMILES: c1ccc(-c2nc(-c3ccccc3)nc(-n3c4ccccc4c4c5oc6ccccc6c5ccc43)n2)cc1 ; c1ccc(-c2cccc(-c3nc(-c4ccccc4)nc(-n4c5ccccc5c5c6oc7ccccc7c6ccc54)n3)c2)cc1 ; c1ccc(-c2nc(-c3cccc4c3sc3ccccc34)nc(-n3c4ccccc4c4c5oc6ccccc6c5ccc43)n2)cc1 ; c1ccc(-c2ccc(-c3nc(-c4cccc5c4oc4ccccc45)nc(-n4c5ccccc5c5c6oc7ccccc7c6ccc54)n3)cc2)cc1 ; c1ccc(-c2ccc(-c3nc(-c4cccc5c4sc4ccccc45)nc(-n4c5ccccc5c5c6oc7ccccc7c6ccc54)n3)cc2)cc1]

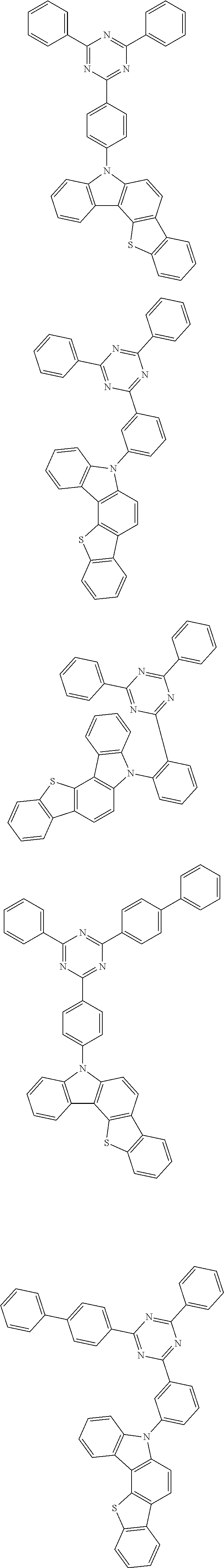

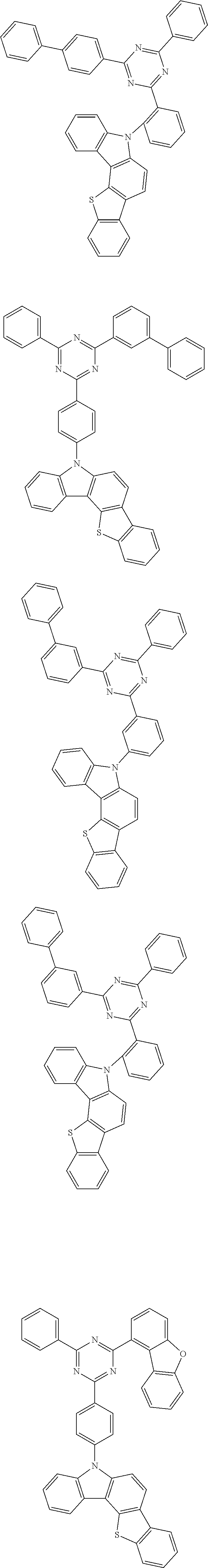 [SMILES: c1ccc(-c2ccc(-c3nc(-c4ccccc4)nc(-c4ccccc4-n4c5ccccc5c5c6sc7ccccc7c6ccc54)n3)cc2)cc1 ; c1ccc(-c2cccc(-c3nc(-c4ccccc4)nc(-c4ccc(-n5c6ccccc6c6c7sc8ccccc8c7ccc65)cc4)n3)c2)cc1 ; c1ccc(-c2cccc(-c3nc(-c4ccccc4)nc(-c4cccc(-n5c6ccccc6c6c7sc8ccccc8c7ccc65)c4)n3)c2)cc1 ; c1ccc(-c2cccc(-c3nc(-c4ccccc4)nc(-c4ccccc4-n4c5ccccc5c5c6sc7ccccc7c6ccc54)n3)c2)cc1 ; c1ccc(-c2nc(-c3ccc(-n4c5ccccc5c5c6sc7ccccc7c6ccc54)cc3)nc(-c3cccc4oc5ccccc5c34)n2)cc1]

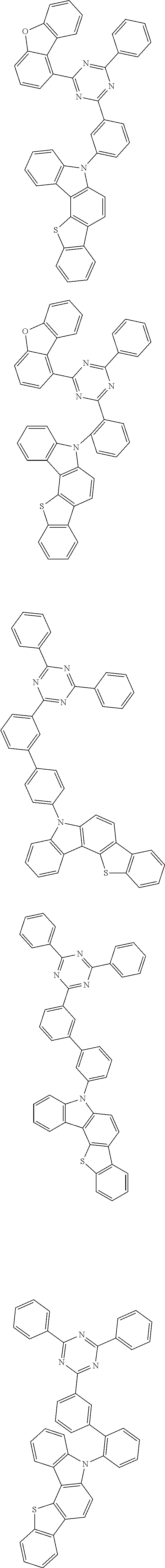

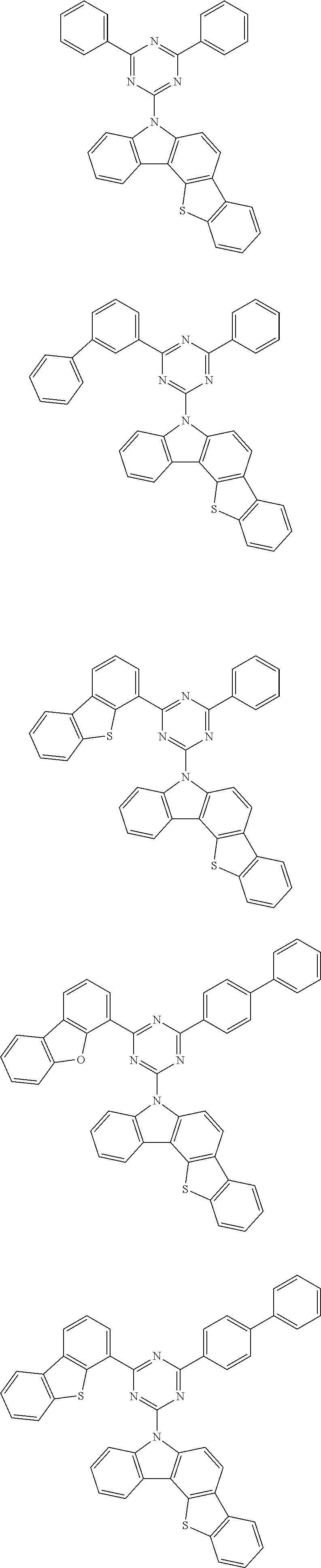 [SMILES: c1ccc(-c2nc(-c3ccccc3)nc(-n3c4ccccc4c4c5sc6ccccc6c5ccc43)n2)cc1 ; c1ccc(-c2cccc(-c3nc(-c4ccccc4)nc(-n4c5ccccc5c5c6sc7ccccc7c6ccc54)n3)c2)cc1 ; c1ccc(-c2nc(-c3cccc4c3sc3ccccc34)nc(-n3c4ccccc4c4c5sc6ccccc6c5ccc43)n2)cc1 ; c1ccc(-c2ccc(-c3nc(-c4cccc5c4oc4ccccc45)nc(-n4c5ccccc5c5c6sc7ccccc7c6ccc54)n3)cc2)cc1 ; c1ccc(-c2ccc(-c3nc(-c4cccc5c4sc4ccccc45)nc(-n4c5ccccc5c5c6sc7ccccc7c6ccc54)n3)cc2)cc1]

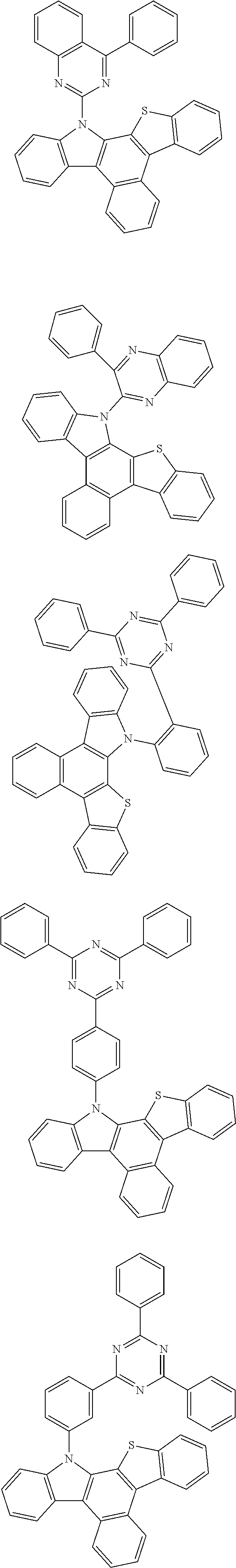

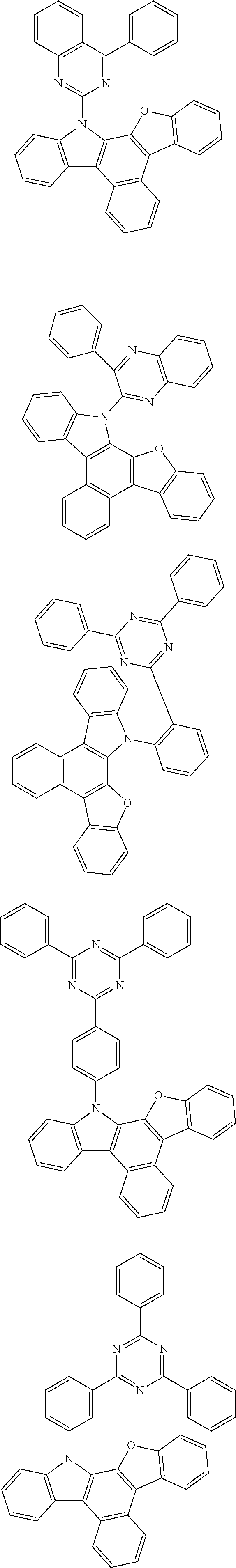

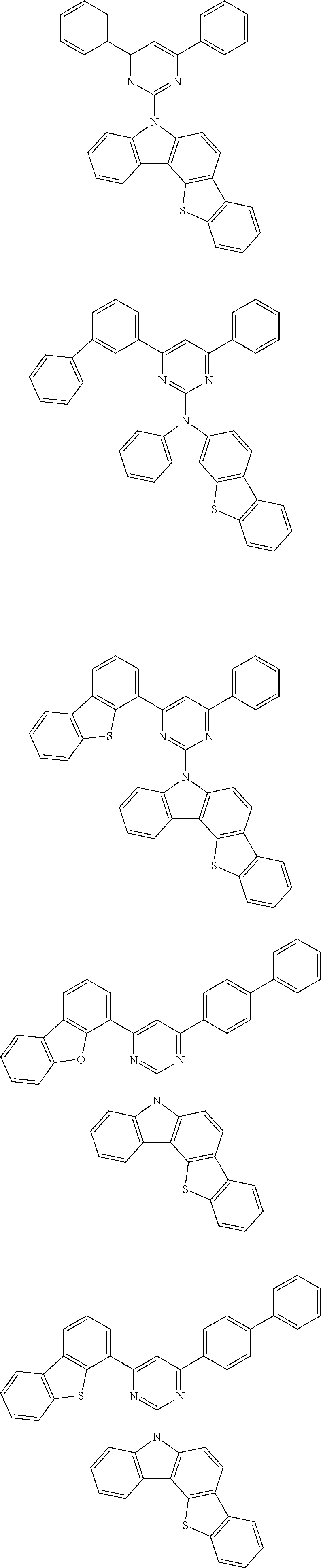 [SMILES: c1ccc(-c2cc(-c3ccccc3)nc(-n3c4ccccc4c4c5sc6ccccc6c5ccc43)n2)cc1 ; c1ccc(-c2cccc(-c3cc(-c4ccccc4)nc(-n4c5ccccc5c5c6sc7ccccc7c6ccc54)n3)c2)cc1 ; c1ccc(-c2cc(-c3cccc4c3sc3ccccc34)nc(-n3c4ccccc4c4c5sc6ccccc6c5ccc43)n2)cc1 ; c1ccc(-c2ccc(-c3cc(-c4cccc5c4oc4ccccc45)nc(-n4c5ccccc5c5c6sc7ccccc7c6ccc54)n3)cc2)cc1 ; c1ccc(-c2ccc(-c3cc(-c4cccc5c4sc4ccccc45)nc(-n4c5ccccc5c5c6sc7ccccc7c6ccc54)n3)cc2)cc1]

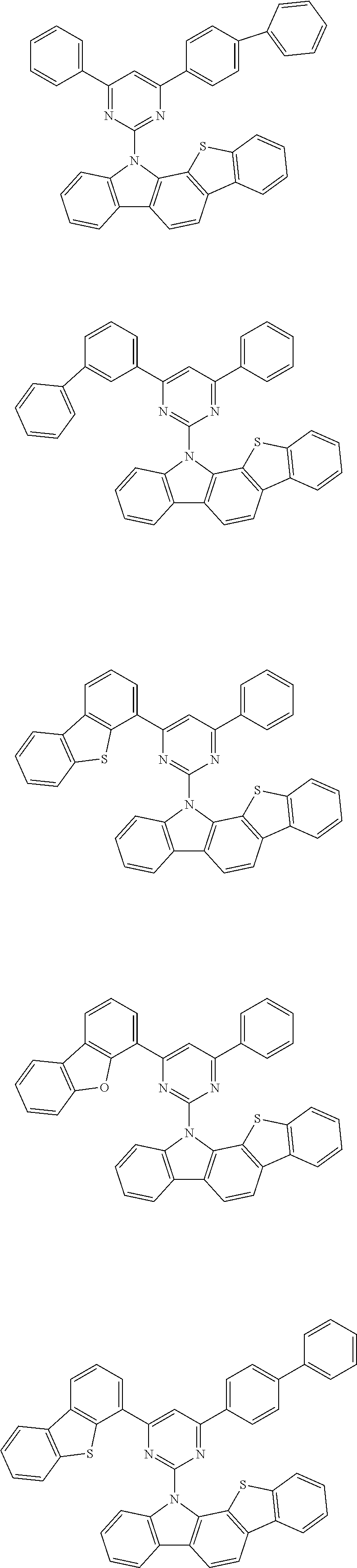 [SMILES: c1ccc(-c2ccc(-c3cc(-c4ccccc4)nc(-n4c5ccccc5c5ccc6c7ccccc7sc6c54)n3)cc2)cc1 ; c1ccc(-c2cccc(-c3cc(-c4ccccc4)nc(-n4c5ccccc5c5ccc6c7ccccc7sc6c54)n3)c2)cc1 ; c1ccc(-c2cc(-c3cccc4c3sc3ccccc34)nc(-n3c4ccccc4c4ccc5c6ccccc6sc5c43)n2)cc1 ; c1ccc(-c2cc(-c3cccc4c3oc3ccccc34)nc(-n3c4ccccc4c4ccc5c6ccccc6sc5c43)n2)cc1 ; c1ccc(-c2ccc(-c3cc(-c4cccc5c4sc4ccccc45)nc(-n4c5ccccc5c5ccc6c7ccccc7sc6c54)n3)cc2)cc1]

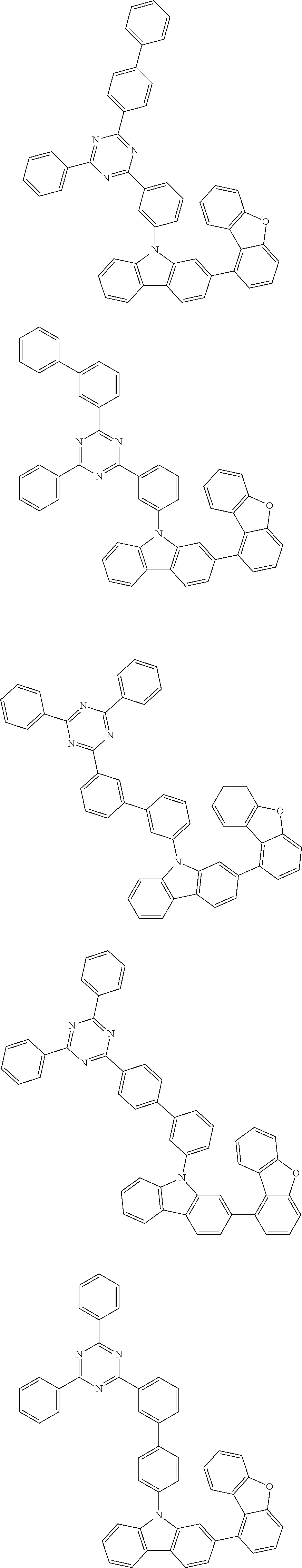 [SMILES: c1ccc(-c2ccc(-c3nc(-c4ccccc4)nc(-c4cccc(-n5c6ccccc6c6ccc(-c7cccc8oc9ccccc9c78)cc65)c4)n3)cc2)cc1 ; c1ccc(-c2cccc(-c3nc(-c4ccccc4)nc(-c4cccc(-n5c6ccccc6c6ccc(-c7cccc8oc9ccccc9c78)cc65)c4)n3)c2)cc1 ; c1ccc(-c2nc(-c3ccccc3)nc(-c3cccc(-c4cccc(-n5c6ccccc6c6ccc(-c7cccc8oc9ccccc9c78)cc65)c4)c3)n2)cc1 ; c1ccc(-c2nc(-c3ccccc3)nc(-c3ccc(-c4cccc(-n5c6ccccc6c6ccc(-c7cccc8oc9ccccc9c78)cc65)c4)cc3)n2)cc1 ; c1ccc(-c2nc(-c3ccccc3)nc(-c3cccc(-c4ccc(-n5c6ccccc6c6ccc(-c7cccc8oc9ccccc9c78)cc65)cc4)c3)n2)cc1]

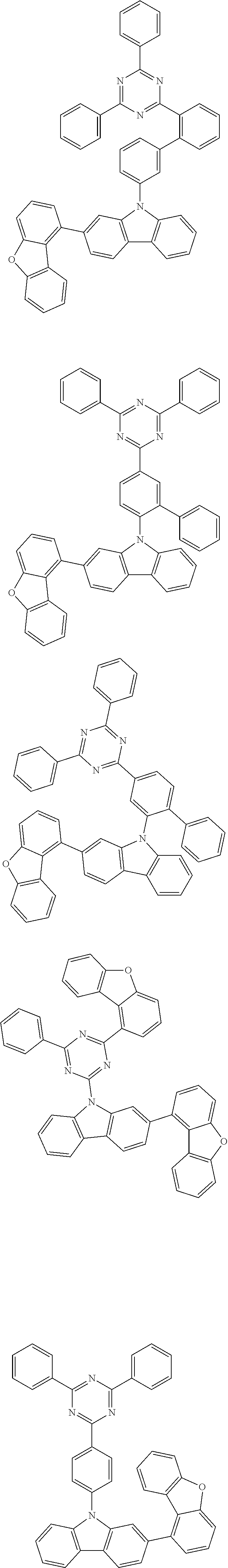 [SMILES: c1ccc(-c2nc(-c3ccccc3)nc(-c3ccccc3-c3cccc(-n4c5ccccc5c5ccc(-c6cccc7oc8ccccc8c67)cc54)c3)n2)cc1 ; c1ccc(-c2nc(-c3ccccc3)nc(-c3ccc(-n4c5ccccc5c5ccc(-c6cccc7oc8ccccc8c67)cc54)c(-c4ccccc4)c3)n2)cc1 ; c1ccc(-c2nc(-c3ccccc3)nc(-c3ccc(-c4ccccc4)c(-n4c5ccccc5c5ccc(-c6cccc7oc8ccccc8c67)cc54)c3)n2)cc1 ; c1ccc(-c2nc(-c3cccc4oc5ccccc5c34)nc(-n3c4ccccc4c4ccc(-c5cccc6oc7ccccc7c56)cc43)n2)cc1 ; c1ccc(-c2nc(-c3ccccc3)nc(-c3ccc(-n4c5ccccc5c5ccc(-c6cccc7oc8ccccc8c67)cc54)cc3)n2)cc1]

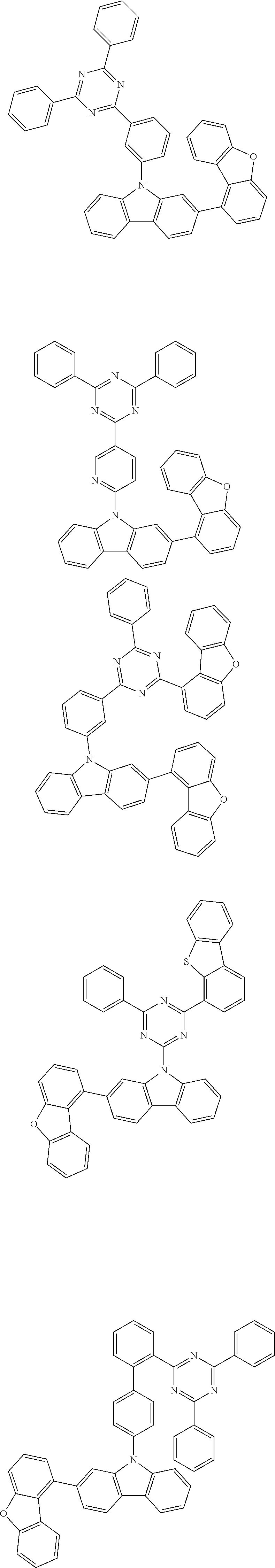

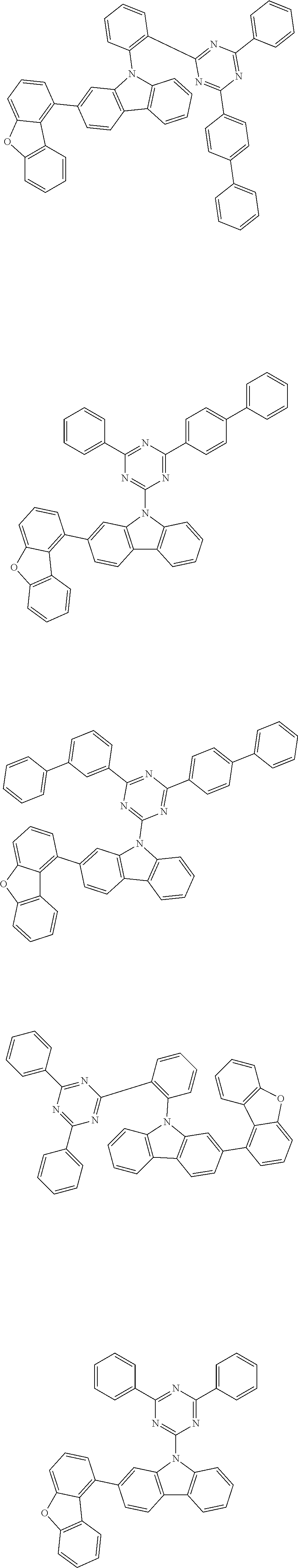 [SMILES: c1ccc(-c2ccc(-c3nc(-c4ccccc4)nc(-c4ccccc4-n4c5ccccc5c5ccc(-c6cccc7oc8ccccc8c67)cc54)n3)cc2)cc1 ; c1ccc(-c2ccc(-c3nc(-c4ccccc4)nc(-n4c5ccccc5c5ccc(-c6cccc7oc8ccccc8c67)cc54)n3)cc2)cc1 ; c1ccc(-c2ccc(-c3nc(-c4cccc(-c5ccccc5)c4)nc(-n4c5ccccc5c5ccc(-c6cccc7oc8ccccc8c67)cc54)n3)cc2)cc1 ; c1ccc(-c2nc(-c3ccccc3)nc(-c3ccccc3-n3c4ccccc4c4ccc(-c5cccc6oc7ccccc7c56)cc43)n2)cc1 ; c1ccc(-c2nc(-c3ccccc3)nc(-n3c4ccccc4c4ccc(-c5cccc6oc7ccccc7c56)cc43)n2)cc1]

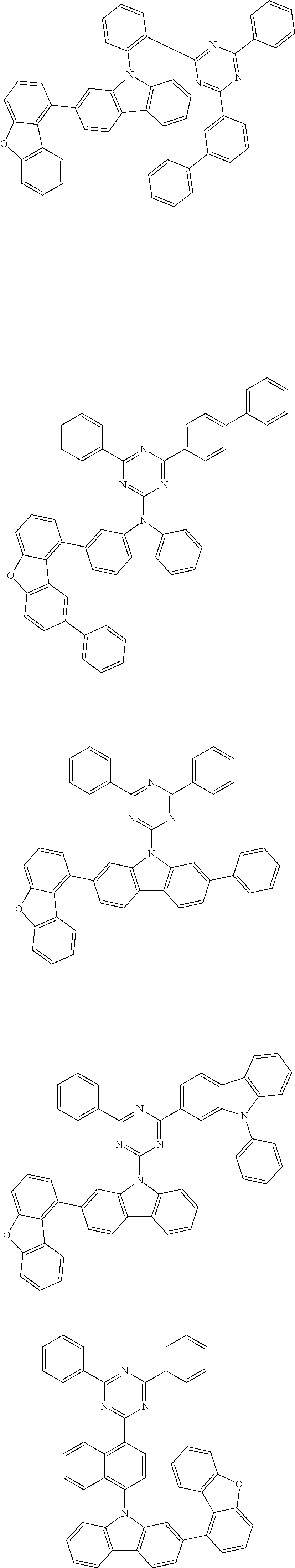 [SMILES: c1ccc(-c2cccc(-c3nc(-c4ccccc4)nc(-c4ccccc4-n4c5ccccc5c5ccc(-c6cccc7oc8ccccc8c67)cc54)n3)c2)cc1 ; c1ccc(-c2ccc(-c3nc(-c4ccccc4)nc(-n4c5ccccc5c5ccc(-c6cccc7oc8ccc(-c9ccccc9)cc8c67)cc54)n3)cc2)cc1 ; c1ccc(-c2ccc3c4ccc(-c5cccc6oc7ccccc7c56)cc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4ccc(-c5cccc6oc7ccccc7c56)cc43)n2)cc1 ; c1ccc(-c2nc(-c3ccccc3)nc(-c3ccc(-n4c5ccccc5c5ccc(-c6cccc7oc8ccccc8c67)cc54)c4ccccc34)n2)cc1]

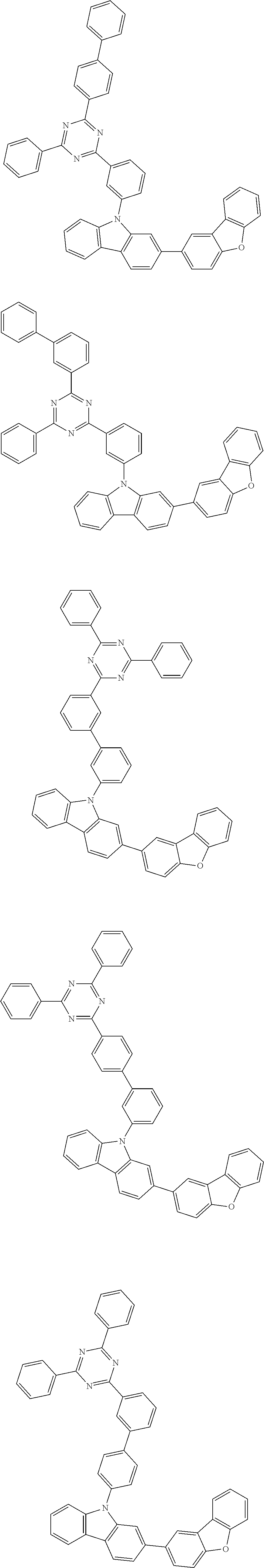 [SMILES: c1ccc(-c2ccc(-c3nc(-c4ccccc4)nc(-c4cccc(-n5c6ccccc6c6ccc(-c7ccc8oc9ccccc9c8c7)cc65)c4)n3)cc2)cc1 ; c1ccc(-c2cccc(-c3nc(-c4ccccc4)nc(-c4cccc(-n5c6ccccc6c6ccc(-c7ccc8oc9ccccc9c8c7)cc65)c4)n3)c2)cc1 ; c1ccc(-c2nc(-c3ccccc3)nc(-c3cccc(-c4cccc(-n5c6ccccc6c6ccc(-c7ccc8oc9ccccc9c8c7)cc65)c4)c3)n2)cc1 ; c1ccc(-c2nc(-c3ccccc3)nc(-c3ccc(-c4cccc(-n5c6ccccc6c6ccc(-c7ccc8oc9ccccc9c8c7)cc65)c4)cc3)n2)cc1 ; c1ccc(-c2nc(-c3ccccc3)nc(-c3cccc(-c4ccc(-n5c6ccccc6c6ccc(-c7ccc8oc9ccccc9c8c7)cc65)cc4)c3)n2)cc1]

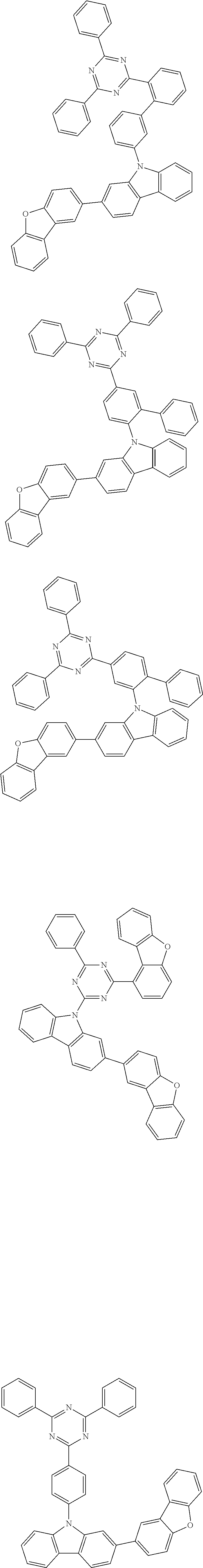 [SMILES: c1ccc(-c2nc(-c3ccccc3)nc(-c3ccccc3-c3cccc(-n4c5ccccc5c5ccc(-c6ccc7oc8ccccc8c7c6)cc54)c3)n2)cc1 ; c1ccc(-c2nc(-c3ccccc3)nc(-c3ccc(-n4c5ccccc5c5ccc(-c6ccc7oc8ccccc8c7c6)cc54)c(-c4ccccc4)c3)n2)cc1 ; c1ccc(-c2nc(-c3ccccc3)nc(-c3ccc(-c4ccccc4)c(-n4c5ccccc5c5ccc(-c6ccc7oc8ccccc8c7c6)cc54)c3)n2)cc1 ; c1ccc(-c2nc(-c3cccc4oc5ccccc5c34)nc(-n3c4ccccc4c4ccc(-c5ccc6oc7ccccc7c6c5)cc43)n2)cc1 ; c1ccc(-c2nc(-c3ccccc3)nc(-c3ccc(-n4c5ccccc5c5ccc(-c6ccc7oc8ccccc8c7c6)cc54)cc3)n2)cc1]

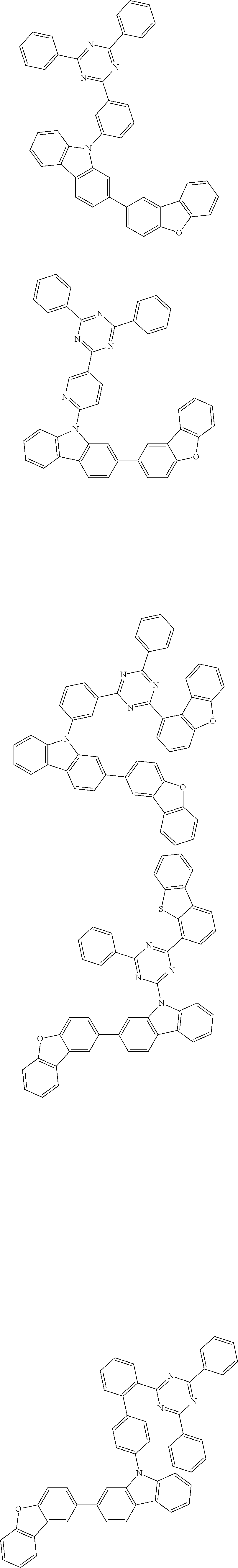

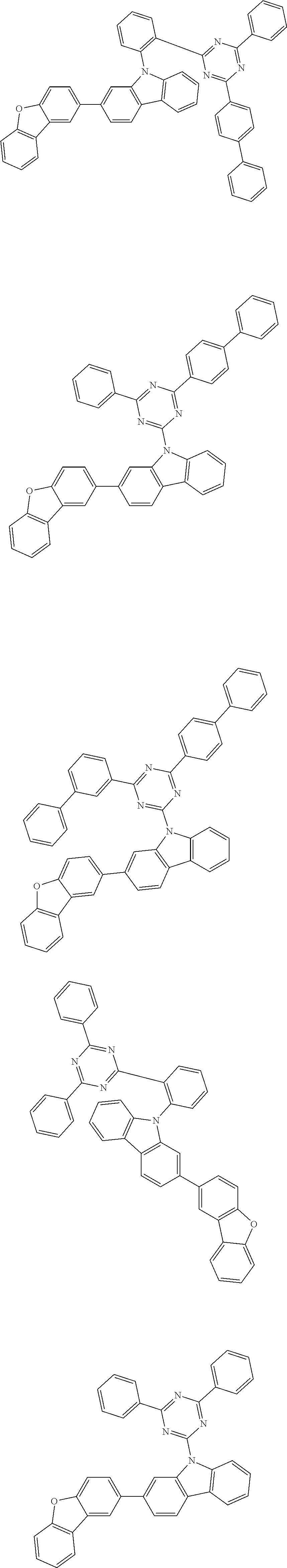 [SMILES: c1ccc(-c2ccc(-c3nc(-c4ccccc4)nc(-c4ccccc4-n4c5ccccc5c5ccc(-c6ccc7oc8ccccc8c7c6)cc54)n3)cc2)cc1 ; c1ccc(-c2ccc(-c3nc(-c4ccccc4)nc(-n4c5ccccc5c5ccc(-c6ccc7oc8ccccc8c7c6)cc54)n3)cc2)cc1 ; c1ccc(-c2ccc(-c3nc(-c4cccc(-c5ccccc5)c4)nc(-n4c5ccccc5c5ccc(-c6ccc7oc8ccccc8c7c6)cc54)n3)cc2)cc1 ; c1ccc(-c2nc(-c3ccccc3)nc(-c3ccccc3-n3c4ccccc4c4ccc(-c5ccc6oc7ccccc7c6c5)cc43)n2)cc1 ; c1ccc(-c2nc(-c3ccccc3)nc(-n3c4ccccc4c4ccc(-c5ccc6oc7ccccc7c6c5)cc43)n2)cc1]

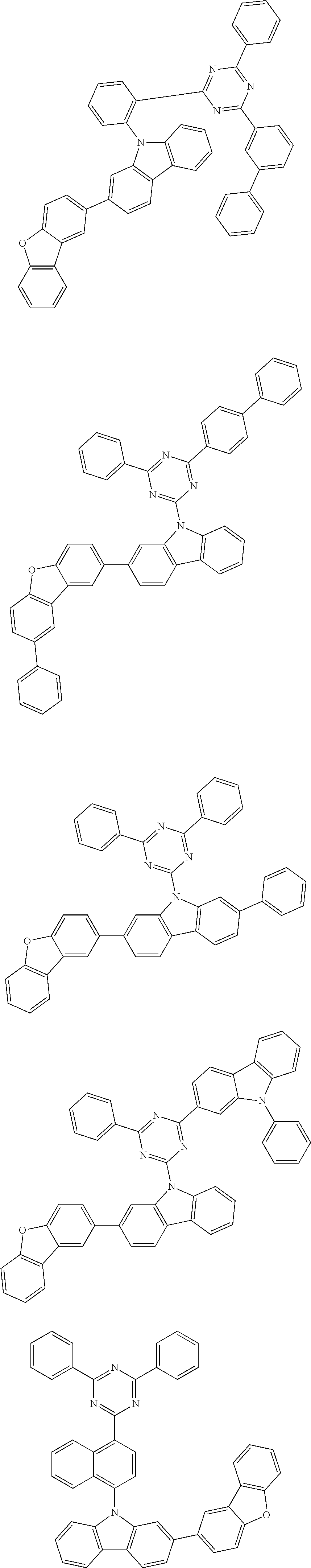 [SMILES: c1ccc(-c2cccc(-c3nc(-c4ccccc4)nc(-c4ccccc4-n4c5ccccc5c5ccc(-c6ccc7oc8ccccc8c7c6)cc54)n3)c2)cc1 ; c1ccc(-c2ccc(-c3nc(-c4ccccc4)nc(-n4c5ccccc5c5ccc(-c6ccc7oc8ccc(-c9ccccc9)cc8c7c6)cc54)n3)cc2)cc1 ; c1ccc(-c2ccc3c4ccc(-c5ccc6oc7ccccc7c6c5)cc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4ccc(-c5ccc6oc7ccccc7c6c5)cc43)n2)cc1 ; c1ccc(-c2nc(-c3ccccc3)nc(-c3ccc(-n4c5ccccc5c5ccc(-c6ccc7oc8ccccc8c7c6)cc54)c4ccccc34)n2)cc1]

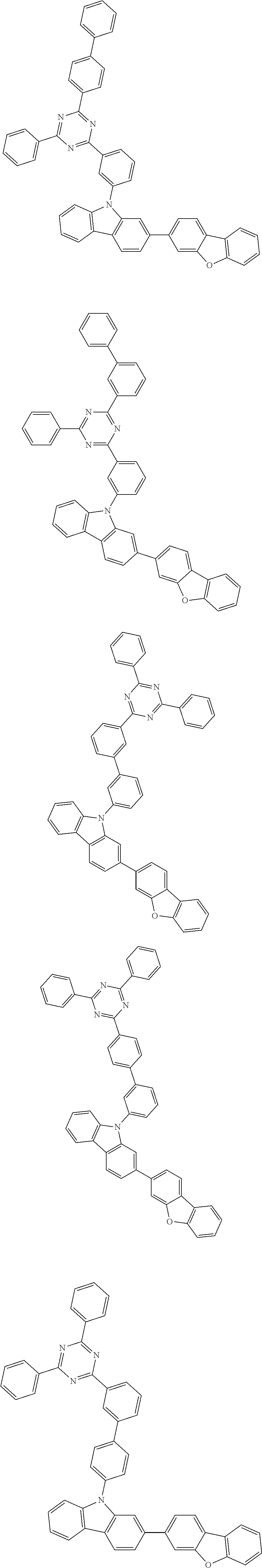

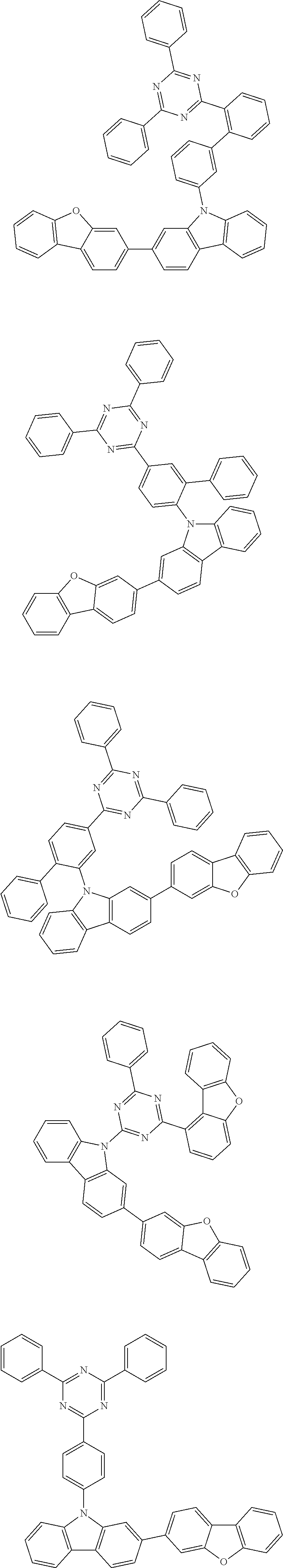 [SMILES: c1ccc(-c2nc(-c3ccccc3)nc(-c3ccccc3-c3cccc(-n4c5ccccc5c5ccc(-c6ccc7c(c6)oc6ccccc67)cc54)c3)n2)cc1 ; c1ccc(-c2nc(-c3ccccc3)nc(-c3ccc(-n4c5ccccc5c5ccc(-c6ccc7c(c6)oc6ccccc67)cc54)c(-c4ccccc4)c3)n2)cc1 ; c1ccc(-c2nc(-c3ccccc3)nc(-c3ccc(-c4ccccc4)c(-n4c5ccccc5c5ccc(-c6ccc7c(c6)oc6ccccc67)cc54)c3)n2)cc1 ; c1ccc(-c2nc(-c3cccc4oc5ccccc5c34)nc(-n3c4ccccc4c4ccc(-c5ccc6c(c5)oc5ccccc56)cc43)n2)cc1 ; c1ccc(-c2nc(-c3ccccc3)nc(-c3ccc(-n4c5ccccc5c5ccc(-c6ccc7c(c6)oc6ccccc67)cc54)cc3)n2)cc1]

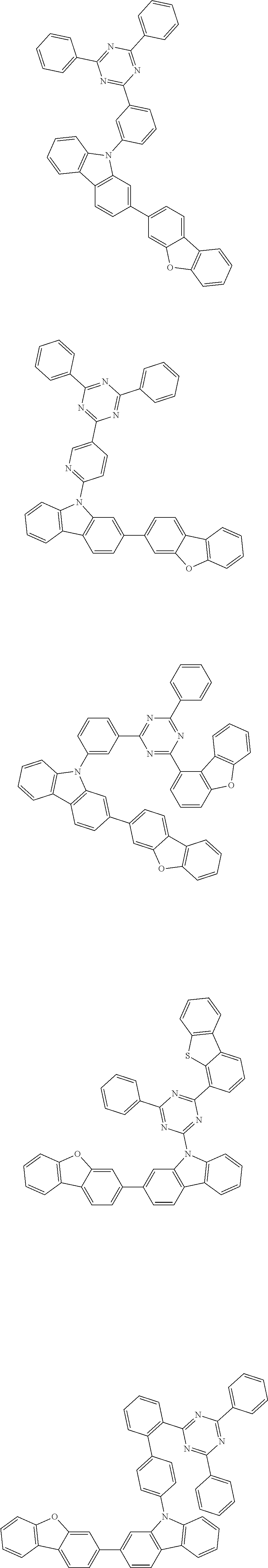 [SMILES: c1ccc(-c2nc(-c3ccccc3)nc(-c3cccc(-n4c5ccccc5c5ccc(-c6ccc7c(c6)oc6ccccc67)cc54)c3)n2)cc1 ; c1ccc(-c2nc(-c3ccccc3)nc(-c3ccc(-n4c5ccccc5c5ccc(-c6ccc7c(c6)oc6ccccc67)cc54)nc3)n2)cc1 ; c1ccc(-c2nc(-c3cccc(-n4c5ccccc5c5ccc(-c6ccc7c(c6)oc6ccccc67)cc54)c3)nc(-c3cccc4oc5ccccc5c34)n2)cc1 ; c1ccc(-c2nc(-c3cccc4c3sc3ccccc34)nc(-n3c4ccccc4c4ccc(-c5ccc6c(c5)oc5ccccc56)cc43)n2)cc1 ; c1ccc(-c2nc(-c3ccccc3)nc(-c3ccccc3-c3ccc(-n4c5ccccc5c5ccc(-c6ccc7c(c6)oc6ccccc67)cc54)cc3)n2)cc1]

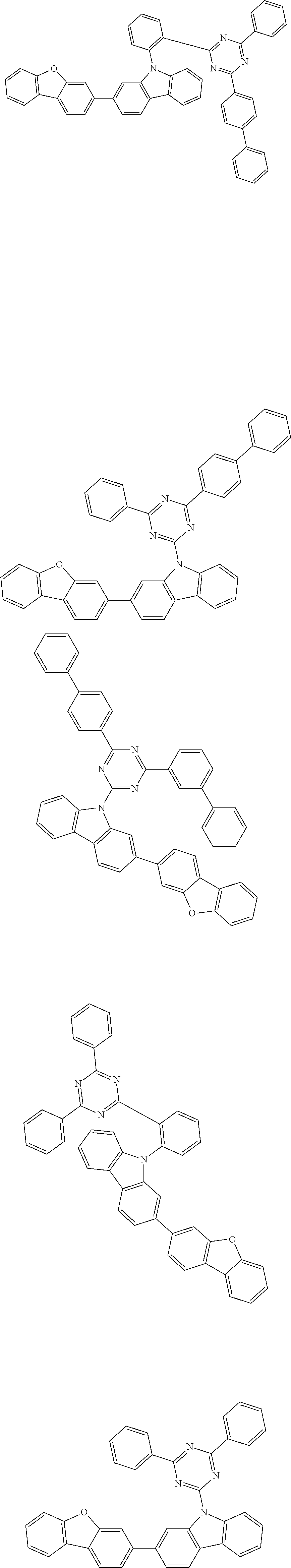

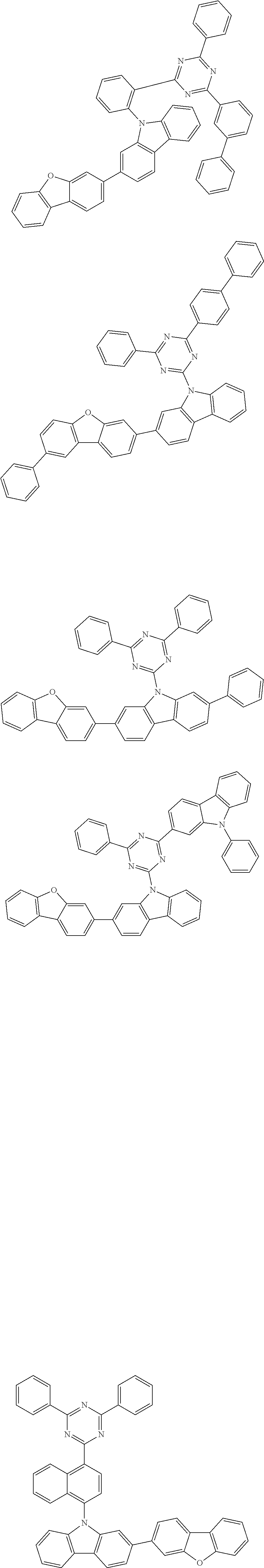 [SMILES: c1ccc(-c2cccc(-c3nc(-c4ccccc4)nc(-c4ccccc4-n4c5ccccc5c5ccc(-c6ccc7c(c6)oc6ccccc67)cc54)n3)c2)cc1 ; c1ccc(-c2ccc(-c3nc(-c4ccccc4)nc(-n4c5ccccc5c5ccc(-c6ccc7c(c6)oc6ccc(-c8ccccc8)cc67)cc54)n3)cc2)cc1 ; c1ccc(-c2ccc3c4ccc(-c5ccc6c(c5)oc5ccccc56)cc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4ccc(-c5ccc6c(c5)oc5ccccc56)cc43)n2)cc1 ; c1ccc(-c2nc(-c3ccccc3)nc(-c3ccc(-n4c5ccccc5c5ccc(-c6ccc7c(c6)oc6ccccc67)cc54)c4ccccc34)n2)cc1]

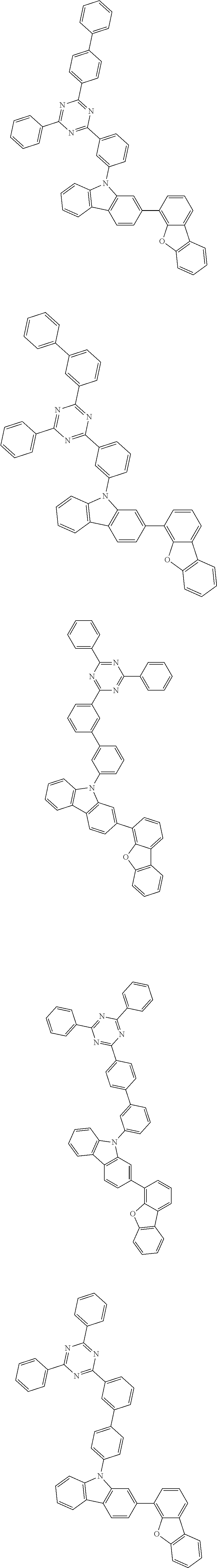 [SMILES: c1ccc(-c2ccc(-c3nc(-c4ccccc4)nc(-c4cccc(-n5c6ccccc6c6ccc(-c7cccc8c7oc7ccccc78)cc65)c4)n3)cc2)cc1 ; c1ccc(-c2cccc(-c3nc(-c4ccccc4)nc(-c4cccc(-n5c6ccccc6c6ccc(-c7cccc8c7oc7ccccc78)cc65)c4)n3)c2)cc1 ; c1ccc(-c2nc(-c3ccccc3)nc(-c3cccc(-c4cccc(-n5c6ccccc6c6ccc(-c7cccc8c7oc7ccccc78)cc65)c4)c3)n2)cc1 ; c1ccc(-c2nc(-c3ccccc3)nc(-c3ccc(-c4cccc(-n5c6ccccc6c6ccc(-c7cccc8c7oc7ccccc78)cc65)c4)cc3)n2)cc1 ; c1ccc(-c2nc(-c3ccccc3)nc(-c3cccc(-c4ccc(-n5c6ccccc6c6ccc(-c7cccc8c7oc7ccccc78)cc65)cc4)c3)n2)cc1]

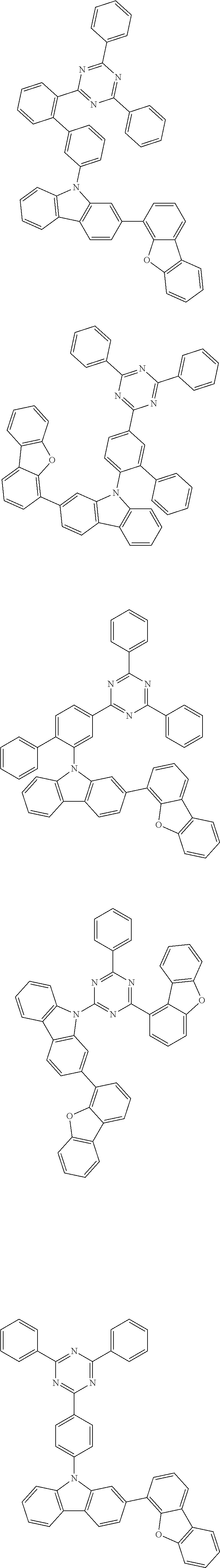 [SMILES: c1ccc(-c2nc(-c3ccccc3)nc(-c3ccccc3-c3cccc(-n4c5ccccc5c5ccc(-c6cccc7c6oc6ccccc67)cc54)c3)n2)cc1 ; c1ccc(-c2nc(-c3ccccc3)nc(-c3ccc(-n4c5ccccc5c5ccc(-c6cccc7c6oc6ccccc67)cc54)c(-c4ccccc4)c3)n2)cc1 ; c1ccc(-c2nc(-c3ccccc3)nc(-c3ccc(-c4ccccc4)c(-n4c5ccccc5c5ccc(-c6cccc7c6oc6ccccc67)cc54)c3)n2)cc1 ; c1ccc(-c2nc(-c3cccc4oc5ccccc5c34)nc(-n3c4ccccc4c4ccc(-c5cccc6c5oc5ccccc56)cc43)n2)cc1 ; c1ccc(-c2nc(-c3ccccc3)nc(-c3ccc(-n4c5ccccc5c5ccc(-c6cccc7c6oc6ccccc67)cc54)cc3)n2)cc1]

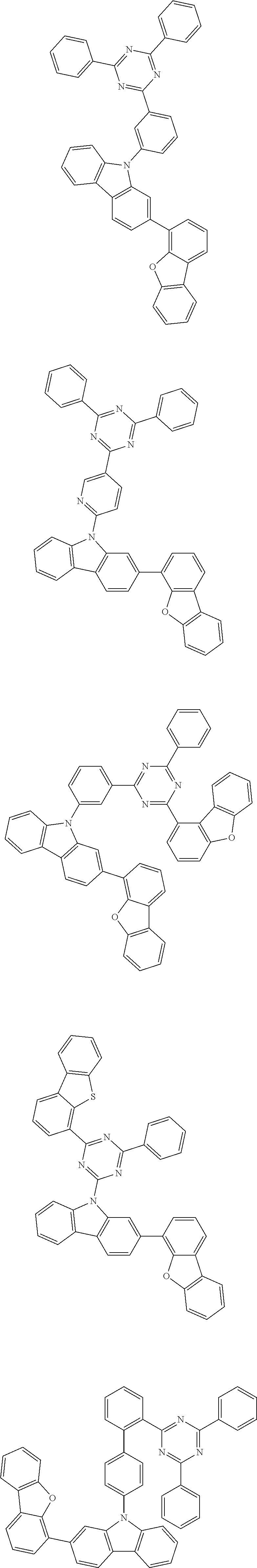 [SMILES: c1ccc(-c2nc(-c3ccccc3)nc(-c3cccc(-n4c5ccccc5c5ccc(-c6cccc7c6oc6ccccc67)cc54)c3)n2)cc1 ; c1ccc(-c2nc(-c3ccccc3)nc(-c3ccc(-n4c5ccccc5c5ccc(-c6cccc7c6oc6ccccc67)cc54)nc3)n2)cc1 ; c1ccc(-c2nc(-c3cccc(-n4c5ccccc5c5ccc(-c6cccc7c6oc6ccccc67)cc54)c3)nc(-c3cccc4oc5ccccc5c34)n2)cc1 ; c1ccc(-c2nc(-c3cccc4c3sc3ccccc34)nc(-n3c4ccccc4c4ccc(-c5cccc6c5oc5ccccc56)cc43)n2)cc1 ; c1ccc(-c2nc(-c3ccccc3)nc(-c3ccccc3-c3ccc(-n4c5ccccc5c5ccc(-c6cccc7c6oc6ccccc67)cc54)cc3)n2)cc1]

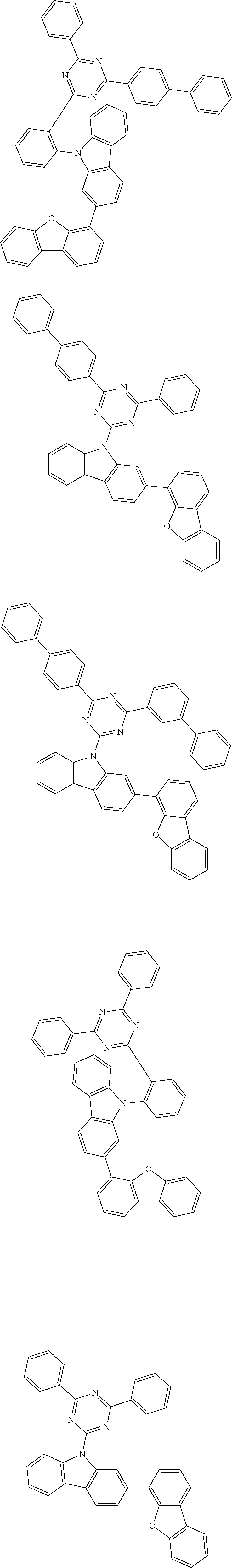 [SMILES: c1ccc(-c2ccc(-c3nc(-c4ccccc4)nc(-c4ccccc4-n4c5ccccc5c5ccc(-c6cccc7c6oc6ccccc67)cc54)n3)cc2)cc1 ; c1ccc(-c2ccc(-c3nc(-c4ccccc4)nc(-n4c5ccccc5c5ccc(-c6cccc7c6oc6ccccc67)cc54)n3)cc2)cc1 ; c1ccc(-c2ccc(-c3nc(-c4cccc(-c5ccccc5)c4)nc(-n4c5ccccc5c5ccc(-c6cccc7c6oc6ccccc67)cc54)n3)cc2)cc1 ; c1ccc(-c2nc(-c3ccccc3)nc(-c3ccccc3-n3c4ccccc4c4ccc(-c5cccc6c5oc5ccccc56)cc43)n2)cc1 ; c1ccc(-c2nc(-c3ccccc3)nc(-n3c4ccccc4c4ccc(-c5cccc6c5oc5ccccc56)cc43)n2)cc1]

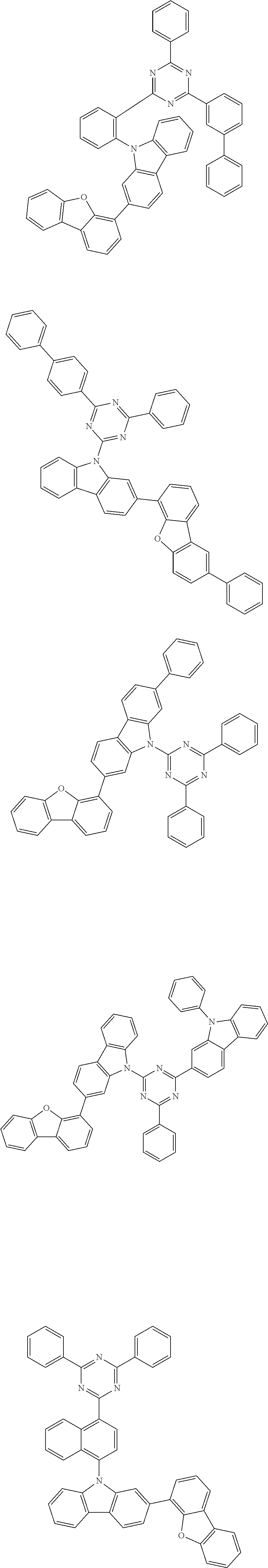 [SMILES: c1ccc(-c2cccc(-c3nc(-c4ccccc4)nc(-c4ccccc4-n4c5ccccc5c5ccc(-c6cccc7c6oc6ccccc67)cc54)n3)c2)cc1 ; c1ccc(-c2ccc(-c3nc(-c4ccccc4)nc(-n4c5ccccc5c5ccc(-c6cccc7c6oc6ccc(-c8ccccc8)cc67)cc54)n3)cc2)cc1 ; c1ccc(-c2ccc3c4ccc(-c5cccc6c5oc5ccccc56)cc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4ccc(-c5cccc6c5oc5ccccc56)cc43)n2)cc1 ; c1ccc(-c2nc(-c3ccccc3)nc(-c3ccc(-n4c5ccccc5c5ccc(-c6cccc7c6oc6ccccc67)cc54)c4ccccc34)n2)cc1]

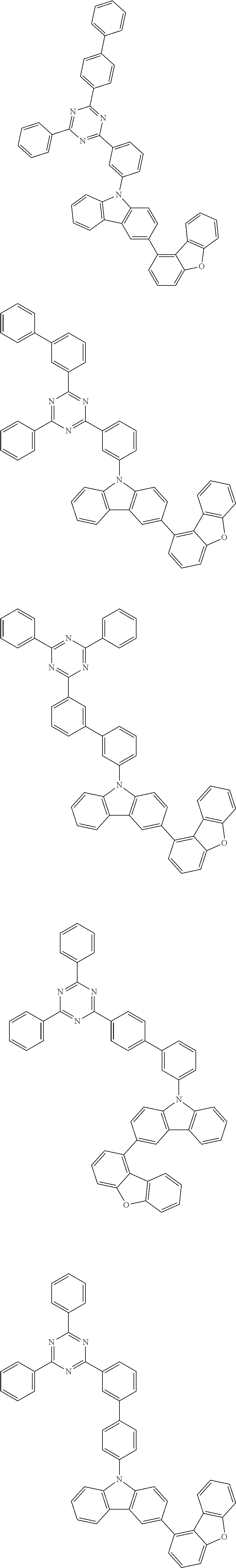 [SMILES: c1ccc(-c2ccc(-c3nc(-c4ccccc4)nc(-c4cccc(-n5c6ccccc6c6cc(-c7cccc8oc9ccccc9c78)ccc65)c4)n3)cc2)cc1 ; c1ccc(-c2cccc(-c3nc(-c4ccccc4)nc(-c4cccc(-n5c6ccccc6c6cc(-c7cccc8oc9ccccc9c78)ccc65)c4)n3)c2)cc1 ; c1ccc(-c2nc(-c3ccccc3)nc(-c3cccc(-c4cccc(-n5c6ccccc6c6cc(-c7cccc8oc9ccccc9c78)ccc65)c4)c3)n2)cc1 ; c1ccc(-c2nc(-c3ccccc3)nc(-c3ccc(-c4cccc(-n5c6ccccc6c6cc(-c7cccc8oc9ccccc9c78)ccc65)c4)cc3)n2)cc1 ; c1ccc(-c2nc(-c3ccccc3)nc(-c3cccc(-c4ccc(-n5c6ccccc6c6cc(-c7cccc8oc9ccccc9c78)ccc65)cc4)c3)n2)cc1]

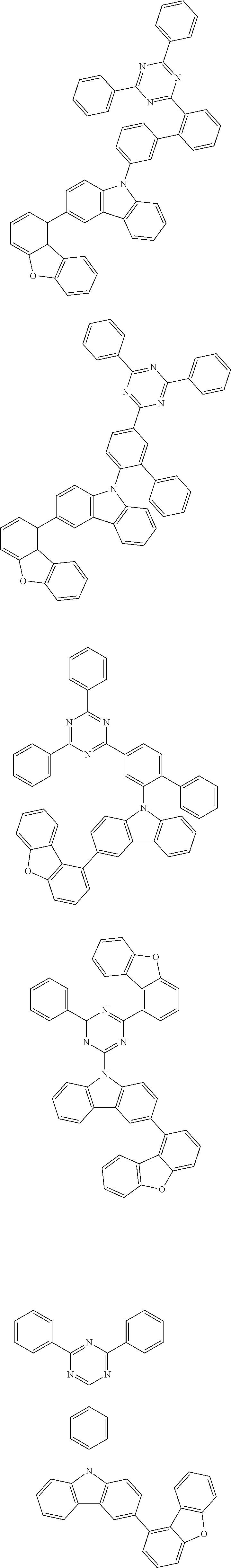 [SMILES: c1ccc(-c2nc(-c3ccccc3)nc(-c3ccccc3-c3cccc(-n4c5ccccc5c5cc(-c6cccc7oc8ccccc8c67)ccc54)c3)n2)cc1 ; c1ccc(-c2nc(-c3ccccc3)nc(-c3ccc(-n4c5ccccc5c5cc(-c6cccc7oc8ccccc8c67)ccc54)c(-c4ccccc4)c3)n2)cc1 ; c1ccc(-c2nc(-c3ccccc3)nc(-c3ccc(-c4ccccc4)c(-n4c5ccccc5c5cc(-c6cccc7oc8ccccc8c67)ccc54)c3)n2)cc1 ; c1ccc(-c2nc(-c3cccc4oc5ccccc5c34)nc(-n3c4ccccc4c4cc(-c5cccc6oc7ccccc7c56)ccc43)n2)cc1 ; c1ccc(-c2nc(-c3ccccc3)nc(-c3ccc(-n4c5ccccc5c5cc(-c6cccc7oc8ccccc8c67)ccc54)cc3)n2)cc1]

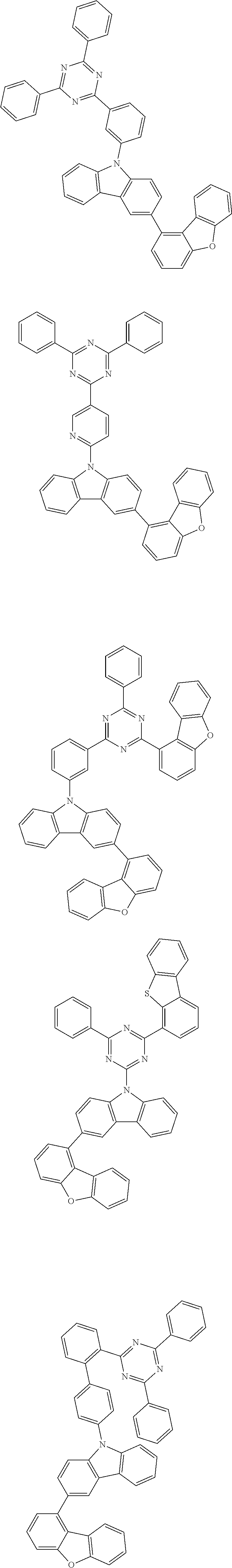 [SMILES: c1ccc(-c2nc(-c3ccccc3)nc(-c3cccc(-n4c5ccccc5c5cc(-c6cccc7oc8ccccc8c67)ccc54)c3)n2)cc1 ; c1ccc(-c2nc(-c3ccccc3)nc(-c3ccc(-n4c5ccccc5c5cc(-c6cccc7oc8ccccc8c67)ccc54)nc3)n2)cc1 ; c1ccc(-c2nc(-c3cccc(-n4c5ccccc5c5cc(-c6cccc7oc8ccccc8c67)ccc54)c3)nc(-c3cccc4oc5ccccc5c34)n2)cc1 ; c1ccc(-c2nc(-c3cccc4c3sc3ccccc34)nc(-n3c4ccccc4c4cc(-c5cccc6oc7ccccc7c56)ccc43)n2)cc1 ; c1ccc(-c2nc(-c3ccccc3)nc(-c3ccccc3-c3ccc(-n4c5ccccc5c5cc(-c6cccc7oc8ccccc8c67)ccc54)cc3)n2)cc1]

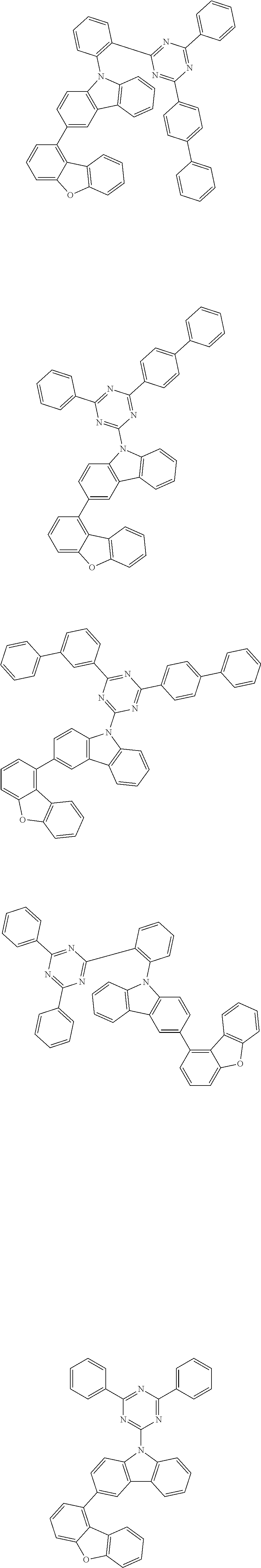 [SMILES: c1ccc(-c2ccc(-c3nc(-c4ccccc4)nc(-c4ccccc4-n4c5ccccc5c5cc(-c6cccc7oc8ccccc8c67)ccc54)n3)cc2)cc1 ; c1ccc(-c2ccc(-c3nc(-c4ccccc4)nc(-n4c5ccccc5c5cc(-c6cccc7oc8ccccc8c67)ccc54)n3)cc2)cc1 ; c1ccc(-c2ccc(-c3nc(-c4cccc(-c5ccccc5)c4)nc(-n4c5ccccc5c5cc(-c6cccc7oc8ccccc8c67)ccc54)n3)cc2)cc1 ; c1ccc(-c2nc(-c3ccccc3)nc(-c3ccccc3-n3c4ccccc4c4cc(-c5cccc6oc7ccccc7c56)ccc43)n2)cc1 ; c1ccc(-c2nc(-c3ccccc3)nc(-n3c4ccccc4c4cc(-c5cccc6oc7ccccc7c56)ccc43)n2)cc1]

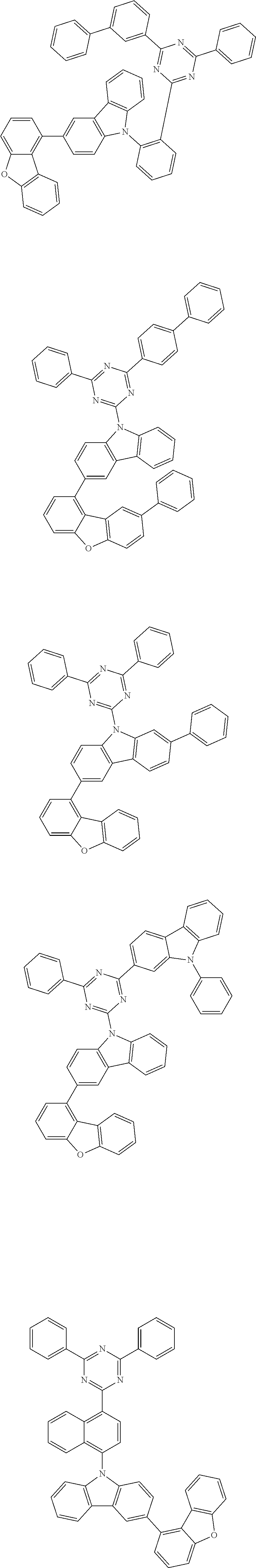 [SMILES: c1ccc(-c2cccc(-c3nc(-c4ccccc4)nc(-c4ccccc4-n4c5ccccc5c5cc(-c6cccc7oc8ccccc8c67)ccc54)n3)c2)cc1 ; c1ccc(-c2ccc(-c3nc(-c4ccccc4)nc(-n4c5ccccc5c5cc(-c6cccc7oc8ccc(-c9ccccc9)cc8c67)ccc54)n3)cc2)cc1 ; c1ccc(-c2ccc3c4cc(-c5cccc6oc7ccccc7c56)ccc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4cc(-c5cccc6oc7ccccc7c56)ccc43)n2)cc1 ; c1ccc(-c2nc(-c3ccccc3)nc(-c3ccc(-n4c5ccccc5c5cc(-c6cccc7oc8ccccc8c67)ccc54)c4ccccc34)n2)cc1]

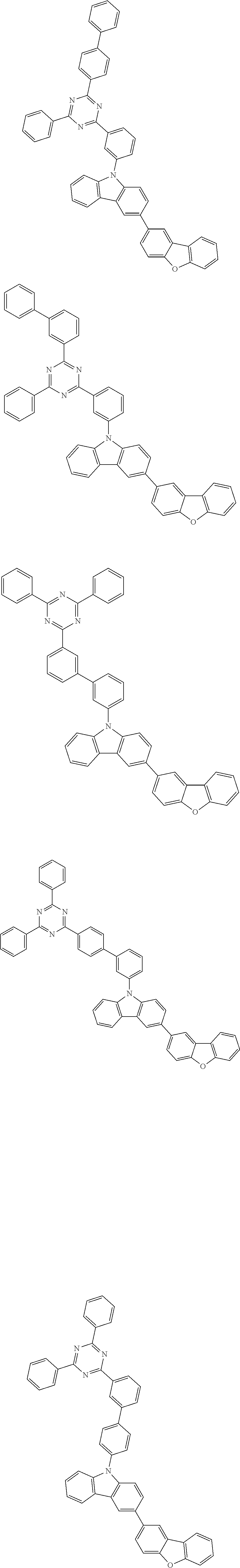 [SMILES: c1ccc(-c2ccc(-c3nc(-c4ccccc4)nc(-c4cccc(-n5c6ccccc6c6cc(-c7ccc8oc9ccccc9c8c7)ccc65)c4)n3)cc2)cc1 ; c1ccc(-c2cccc(-c3nc(-c4ccccc4)nc(-c4cccc(-n5c6ccccc6c6cc(-c7ccc8oc9ccccc9c8c7)ccc65)c4)n3)c2)cc1 ; c1ccc(-c2nc(-c3ccccc3)nc(-c3cccc(-c4cccc(-n5c6ccccc6c6cc(-c7ccc8oc9ccccc9c8c7)ccc65)c4)c3)n2)cc1 ; c1ccc(-c2nc(-c3ccccc3)nc(-c3ccc(-c4cccc(-n5c6ccccc6c6cc(-c7ccc8oc9ccccc9c8c7)ccc65)c4)cc3)n2)cc1 ; c1ccc(-c2nc(-c3ccccc3)nc(-c3cccc(-c4ccc(-n5c6ccccc6c6cc(-c7ccc8oc9ccccc9c8c7)ccc65)cc4)c3)n2)cc1]

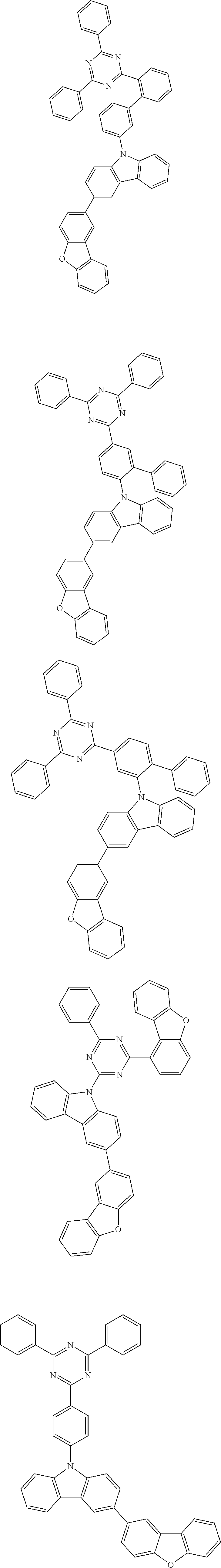

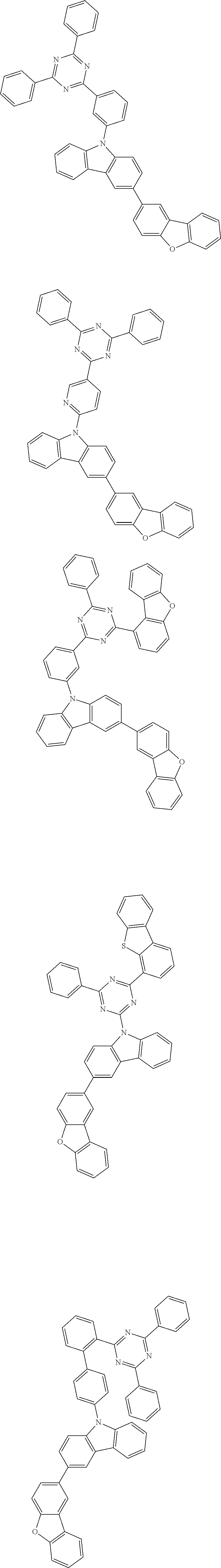 [SMILES: c1ccc(-c2nc(-c3ccccc3)nc(-c3cccc(-n4c5ccccc5c5cc(-c6ccc7oc8ccccc8c7c6)ccc54)c3)n2)cc1 ; c1ccc(-c2nc(-c3ccccc3)nc(-c3ccc(-n4c5ccccc5c5cc(-c6ccc7oc8ccccc8c7c6)ccc54)nc3)n2)cc1 ; c1ccc(-c2nc(-c3cccc(-n4c5ccccc5c5cc(-c6ccc7oc8ccccc8c7c6)ccc54)c3)nc(-c3cccc4oc5ccccc5c34)n2)cc1 ; c1ccc(-c2nc(-c3cccc4c3sc3ccccc34)nc(-n3c4ccccc4c4cc(-c5ccc6oc7ccccc7c6c5)ccc43)n2)cc1 ; c1ccc(-c2nc(-c3ccccc3)nc(-c3ccccc3-c3ccc(-n4c5ccccc5c5cc(-c6ccc7oc8ccccc8c7c6)ccc54)cc3)n2)cc1]

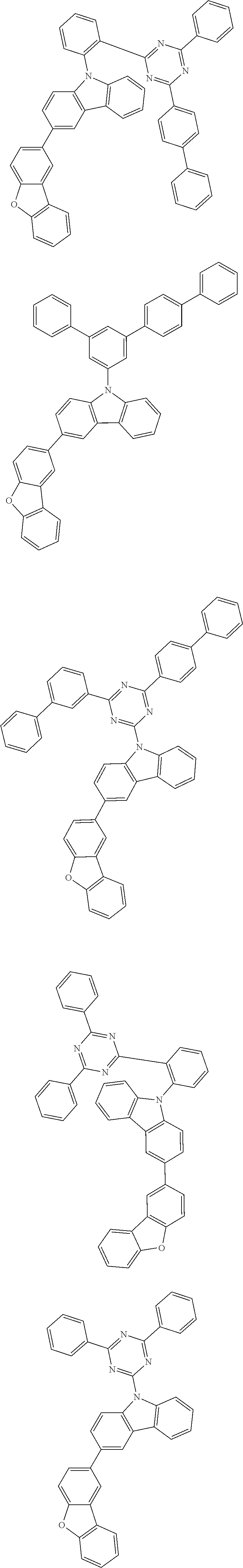 [SMILES: c1ccc(-c2ccc(-c3nc(-c4ccccc4)nc(-c4ccccc4-n4c5ccccc5c5cc(-c6ccc7oc8ccccc8c7c6)ccc54)n3)cc2)cc1 ; c1ccc(-c2ccc(-c3cc(-c4ccccc4)cc(-n4c5ccccc5c5cc(-c6ccc7oc8ccccc8c7c6)ccc54)c3)cc2)cc1 ; c1ccc(-c2ccc(-c3nc(-c4cccc(-c5ccccc5)c4)nc(-n4c5ccccc5c5cc(-c6ccc7oc8ccccc8c7c6)ccc54)n3)cc2)cc1 ; c1ccc(-c2nc(-c3ccccc3)nc(-c3ccccc3-n3c4ccccc4c4cc(-c5ccc6oc7ccccc7c6c5)ccc43)n2)cc1 ; c1ccc(-c2nc(-c3ccccc3)nc(-n3c4ccccc4c4cc(-c5ccc6oc7ccccc7c6c5)ccc43)n2)cc1]

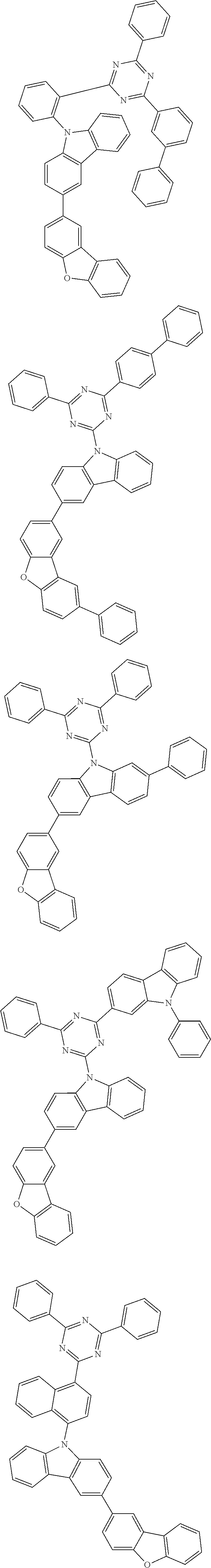

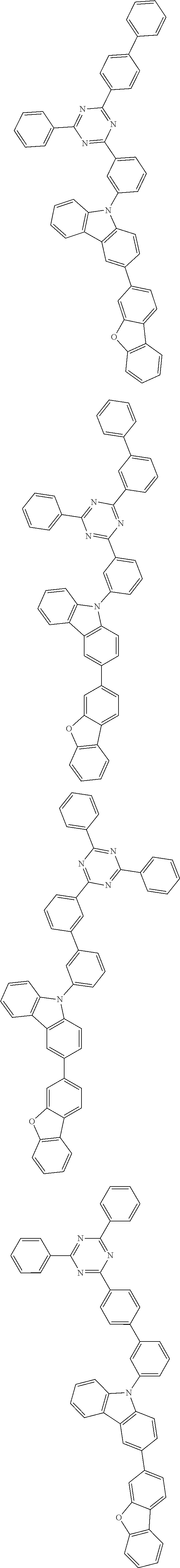

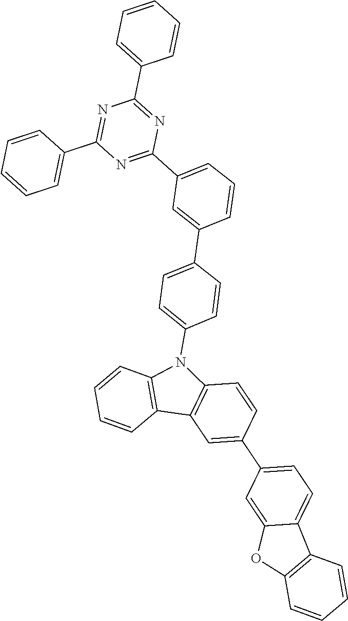 [SMILES: c1ccc(-c2nc(-c3ccccc3)nc(-c3cccc(-c4ccc(-n5c6ccccc6c6cc(-c7ccc8c(c7)oc7ccccc78)ccc65)cc4)c3)n2)cc1]

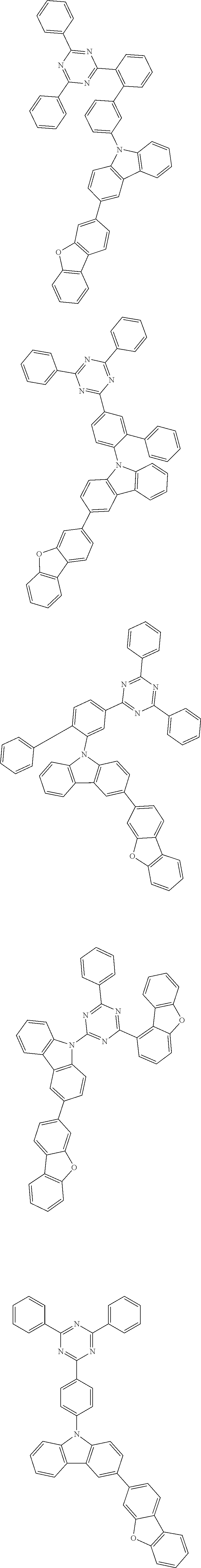 [SMILES: c1ccc(-c2nc(-c3ccccc3)nc(-c3ccccc3-c3cccc(-n4c5ccccc5c5cc(-c6ccc7c(c6)oc6ccccc67)ccc54)c3)n2)cc1 ; c1ccc(-c2nc(-c3ccccc3)nc(-c3ccc(-n4c5ccccc5c5cc(-c6ccc7c(c6)oc6ccccc67)ccc54)c(-c4ccccc4)c3)n2)cc1 ; c1ccc(-c2nc(-c3ccccc3)nc(-c3ccc(-c4ccccc4)c(-n4c5ccccc5c5cc(-c6ccc7c(c6)oc6ccccc67)ccc54)c3)n2)cc1 ; c1ccc(-c2nc(-c3cccc4oc5ccccc5c34)nc(-n3c4ccccc4c4cc(-c5ccc6c(c5)oc5ccccc56)ccc43)n2)cc1 ; c1ccc(-c2nc(-c3ccccc3)nc(-c3ccc(-n4c5ccccc5c5cc(-c6ccc7c(c6)oc6ccccc67)ccc54)cc3)n2)cc1]

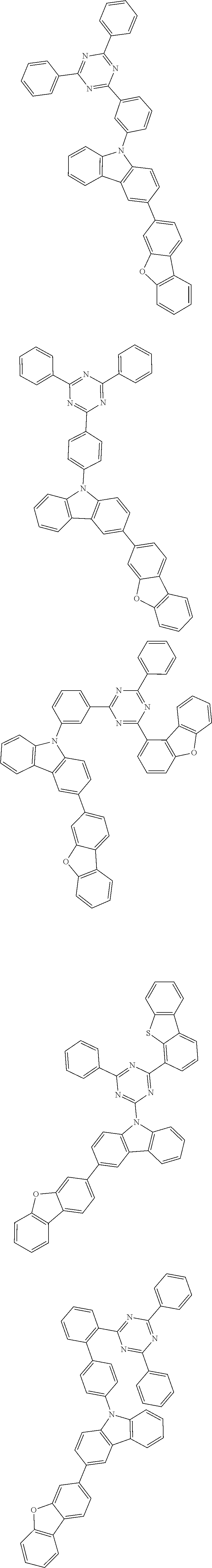

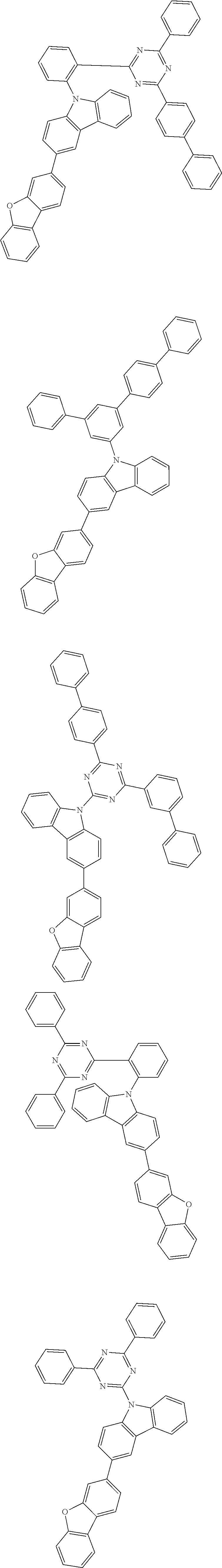

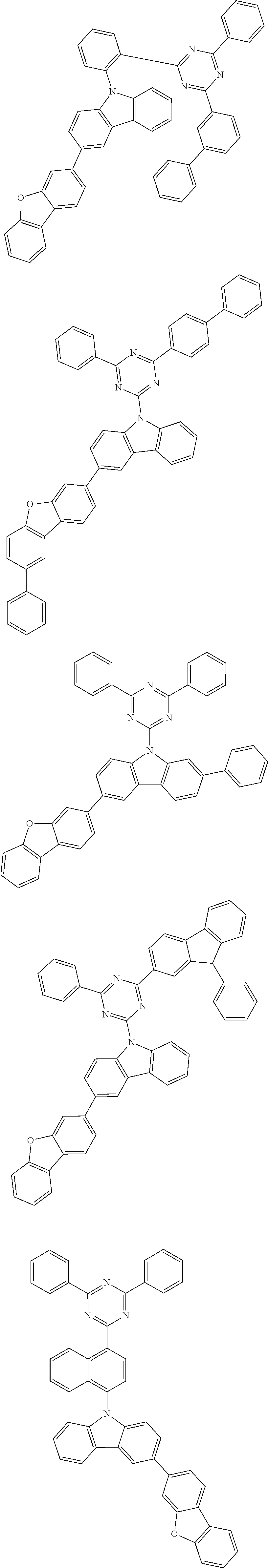 [SMILES: c1ccc(-c2cccc(-c3nc(-c4ccccc4)nc(-c4ccccc4-n4c5ccccc5c5cc(-c6ccc7c(c6)oc6ccccc67)ccc54)n3)c2)cc1 ; c1ccc(-c2ccc(-c3nc(-c4ccccc4)nc(-n4c5ccccc5c5cc(-c6ccc7c(c6)oc6ccc(-c8ccccc8)cc67)ccc54)n3)cc2)cc1 ; c1ccc(-c2ccc3c4cc(-c5ccc6c(c5)oc5ccccc56)ccc4n(-c4nc(-c5ccccc5)nc(-c5ccccc5)n4)c3c2)cc1 ; c1ccc(-c2nc(-c3ccc4c(c3)C(c3ccccc3)c3ccccc3-4)nc(-n3c4ccccc4c4cc(-c5ccc6c(c5)oc5ccccc56)ccc43)n2)cc1 ; c1ccc(-c2nc(-c3ccccc3)nc(-c3ccc(-n4c5ccccc5c5cc(-c6ccc7c(c6)oc6ccccc67)ccc54)c4ccccc34)n2)cc1]

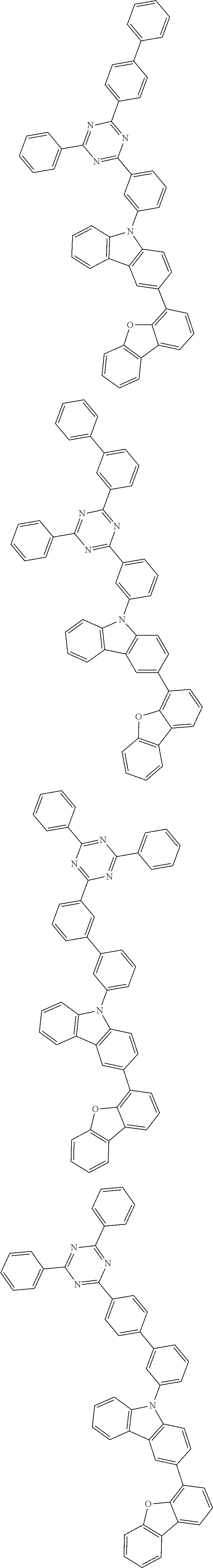

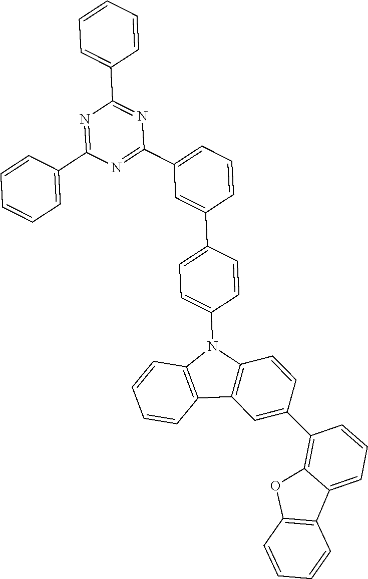 [SMILES: c1ccc(-c2nc(-c3ccccc3)nc(-c3cccc(-c4ccc(-n5c6ccccc6c6cc(-c7cccc8c7oc7ccccc78)ccc65)cc4)c3)n2)cc1]

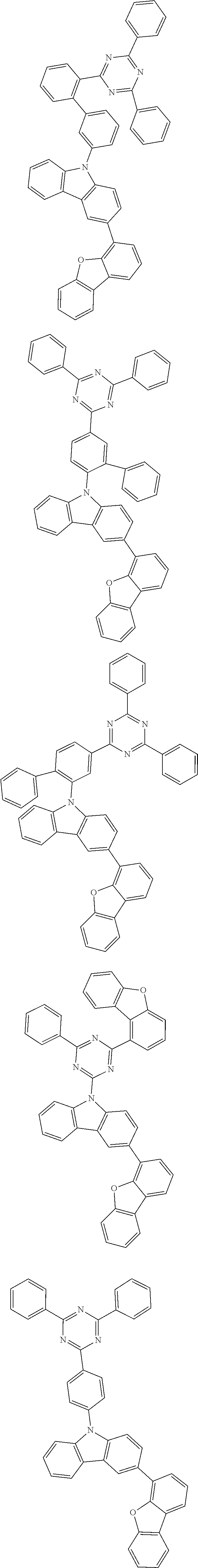

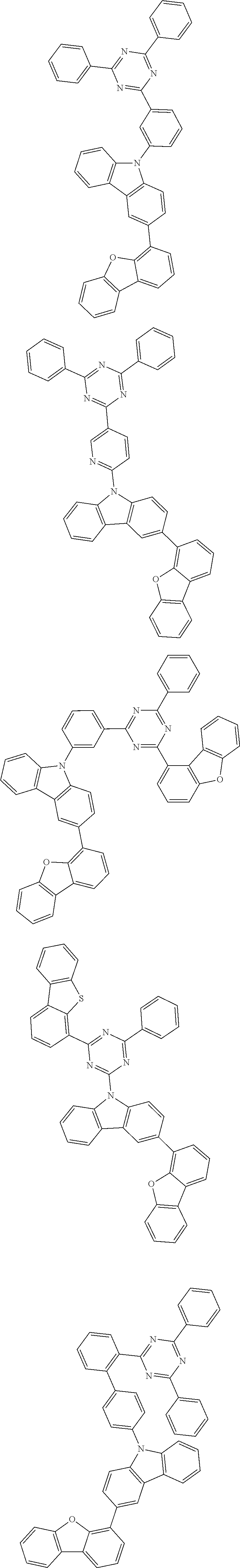

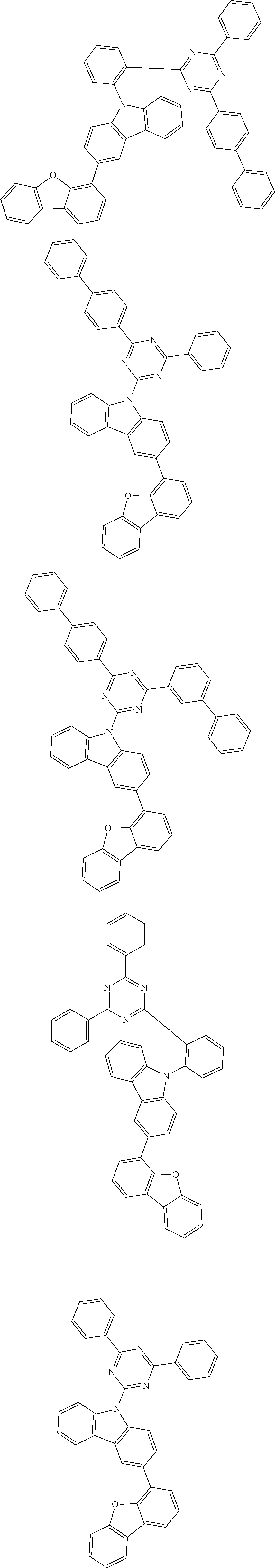

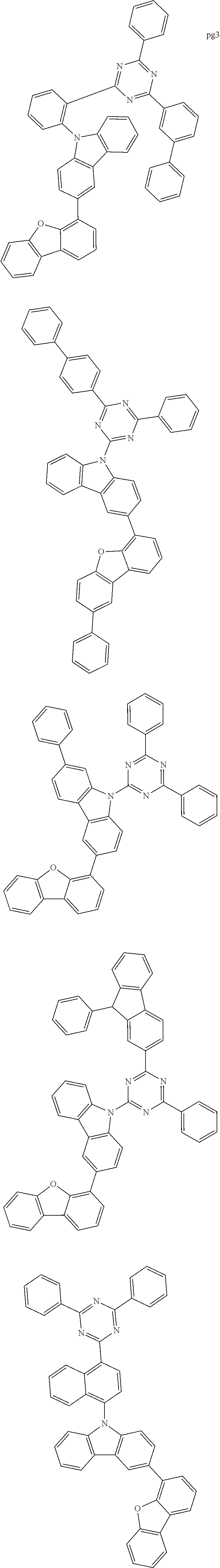 [SMILES: c1ccc(-c2cccc(-c3nc(-c4ccccc4)nc(-c4ccccc4-n4c5ccccc5c5cc(-c6cccc7c6oc6ccccc67)ccc54)n3)c2)cc1 ; c1ccc(-c2ccc(-c3nc(-c4ccccc4)nc(-n4c5ccccc5c5cc(-c6cccc7c6oc6ccc(-c8ccccc8)cc67)ccc54)n3)cc2)cc1 ; c1ccc(-c2ccc3c4cc(-c5cccc6c5oc5ccccc56)ccc4n(-c4nc(-c5ccccc5)nc(-c5ccccc5)n4)c3c2)cc1 ; c1ccc(-c2nc(-c3ccc4c(c3)C(c3ccccc3)c3ccccc3-4)nc(-n3c4ccccc4c4cc(-c5cccc6c5oc5ccccc56)ccc43)n2)cc1 ; c1ccc(-c2nc(-c3ccccc3)nc(-c3ccc(-n4c5ccccc5c5cc(-c6cccc7c6oc6ccccc67)ccc54)c4ccccc34)n2)cc1]

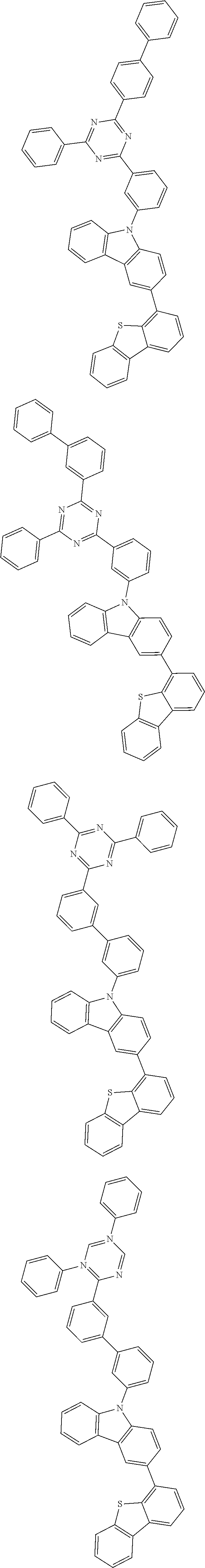

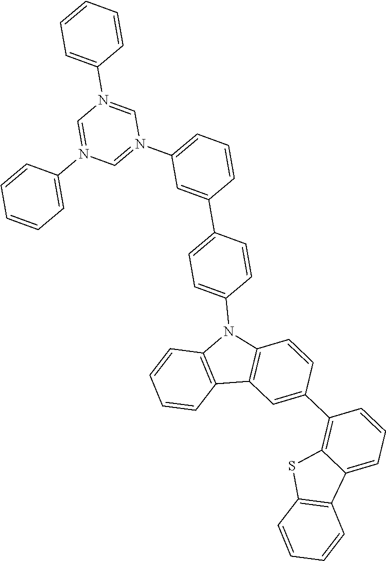

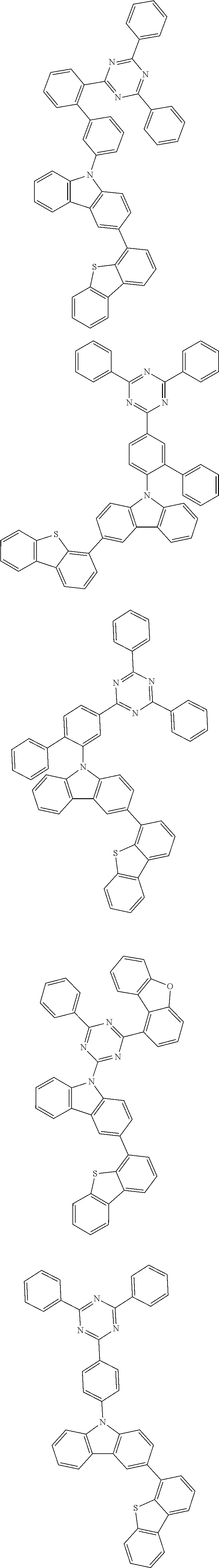

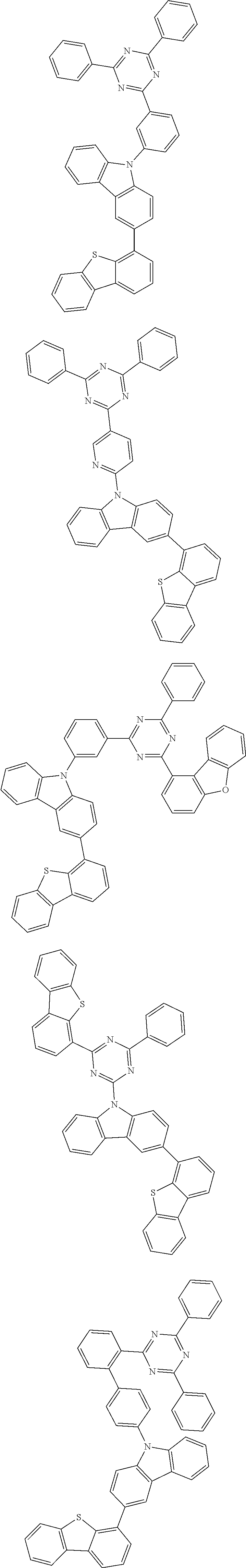

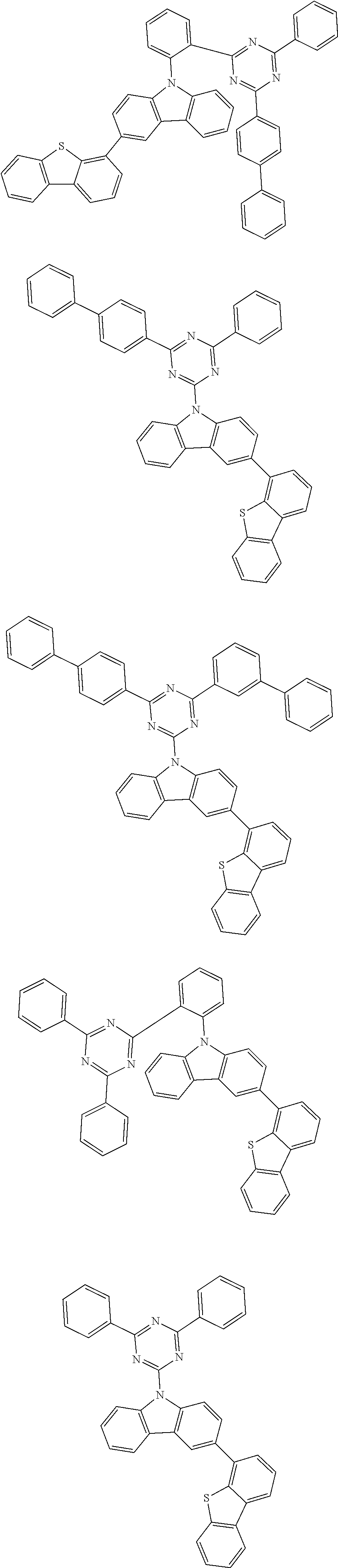 [SMILES: c1ccc(-c2ccc(-c3nc(-c4ccccc4)nc(-c4ccccc4-n4c5ccccc5c5cc(-c6cccc7c6sc6ccccc67)ccc54)n3)cc2)cc1 ; c1ccc(-c2ccc(-c3nc(-c4ccccc4)nc(-n4c5ccccc5c5cc(-c6cccc7c6sc6ccccc67)ccc54)n3)cc2)cc1 ; c1ccc(-c2ccc(-c3nc(-c4cccc(-c5ccccc5)c4)nc(-n4c5ccccc5c5cc(-c6cccc7c6sc6ccccc67)ccc54)n3)cc2)cc1 ; c1ccc(-c2nc(-c3ccccc3)nc(-c3ccccc3-n3c4ccccc4c4cc(-c5cccc6c5sc5ccccc56)ccc43)n2)cc1 ; c1ccc(-c2nc(-c3ccccc3)nc(-n3c4ccccc4c4cc(-c5cccc6c5sc5ccccc56)ccc43)n2)cc1]

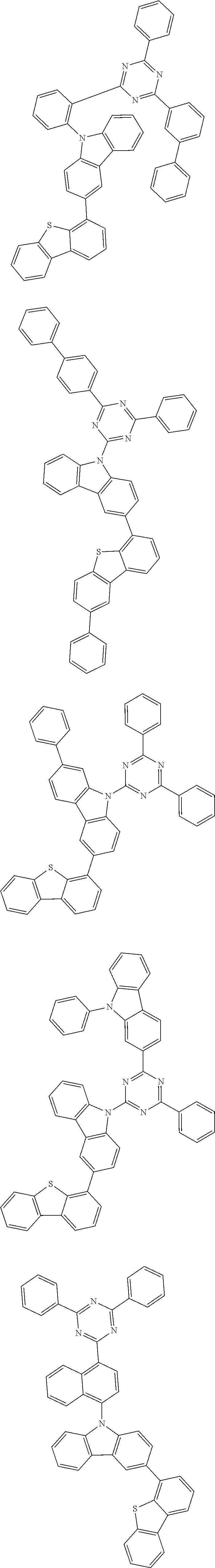 [SMILES: c1ccc(-c2cccc(-c3nc(-c4ccccc4)nc(-c4ccccc4-n4c5ccccc5c5cc(-c6cccc7c6sc6ccccc67)ccc54)n3)c2)cc1 ; c1ccc(-c2ccc(-c3nc(-c4ccccc4)nc(-n4c5ccccc5c5cc(-c6cccc7c6sc6ccc(-c8ccccc8)cc67)ccc54)n3)cc2)cc1 ; c1ccc(-c2ccc3c4cc(-c5cccc6c5sc5ccccc56)ccc4n(-c4nc(-c5ccccc5)nc(-c5ccccc5)n4)c3c2)cc1 ; c1ccc(-c2nc(-c3ccc4c5ccccc5n(-c5ccccc5)c4c3)nc(-n3c4ccccc4c4cc(-c5cccc6c5sc5ccccc56)ccc43)n2)cc1 ; c1ccc(-c2nc(-c3ccccc3)nc(-c3ccc(-n4c5ccccc5c5cc(-c6cccc7c6sc6ccccc67)ccc54)c4ccccc34)n2)cc1]

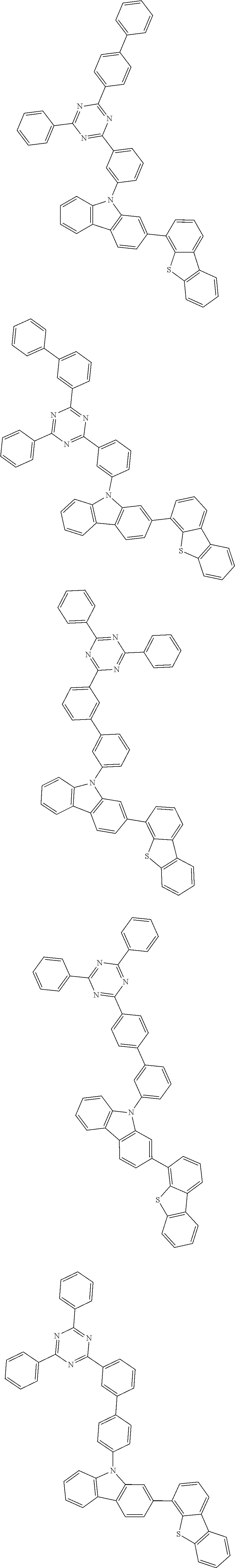 [SMILES: c1ccc(-c2ccc(-c3nc(-c4ccccc4)nc(-c4cccc(-n5c6ccccc6c6ccc(-c7cccc8c7sc7ccccc78)cc65)c4)n3)cc2)cc1 ; c1ccc(-c2cccc(-c3nc(-c4ccccc4)nc(-c4cccc(-n5c6ccccc6c6ccc(-c7cccc8c7sc7ccccc78)cc65)c4)n3)c2)cc1 ; c1ccc(-c2nc(-c3ccccc3)nc(-c3cccc(-c4cccc(-n5c6ccccc6c6ccc(-c7cccc8c7sc7ccccc78)cc65)c4)c3)n2)cc1 ; c1ccc(-c2nc(-c3ccccc3)nc(-c3ccc(-c4cccc(-n5c6ccccc6c6ccc(-c7cccc8c7sc7ccccc78)cc65)c4)cc3)n2)cc1 ; c1ccc(-c2nc(-c3ccccc3)nc(-c3cccc(-c4ccc(-n5c6ccccc6c6ccc(-c7cccc8c7sc7ccccc78)cc65)cc4)c3)n2)cc1]

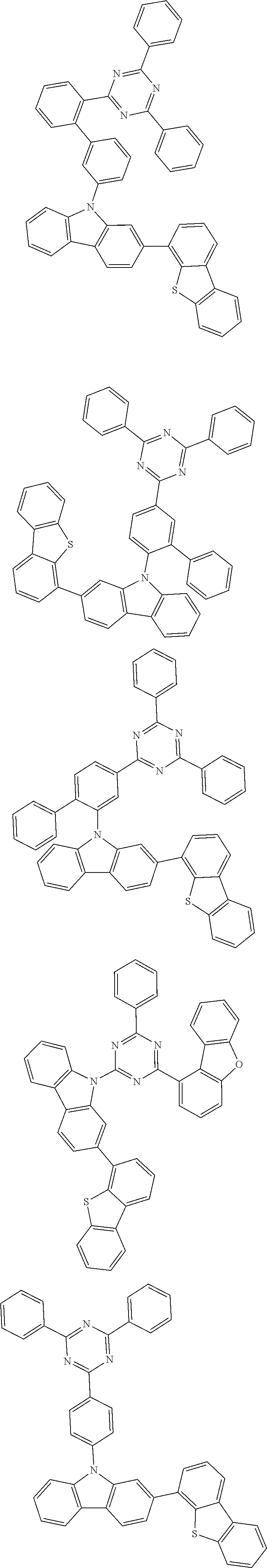

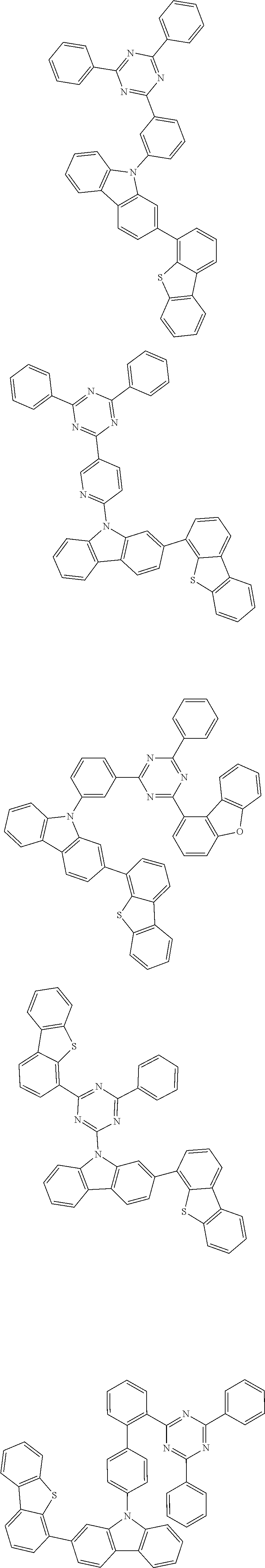

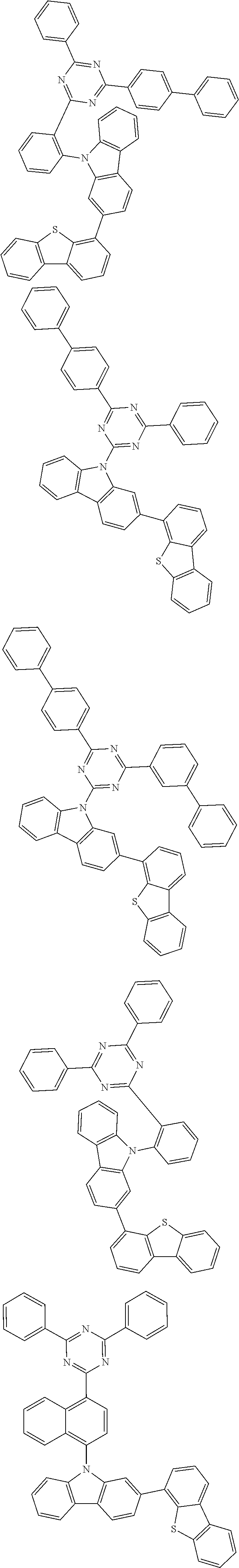

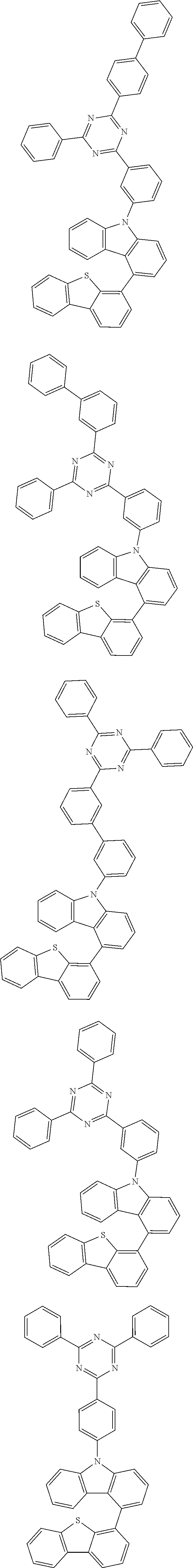

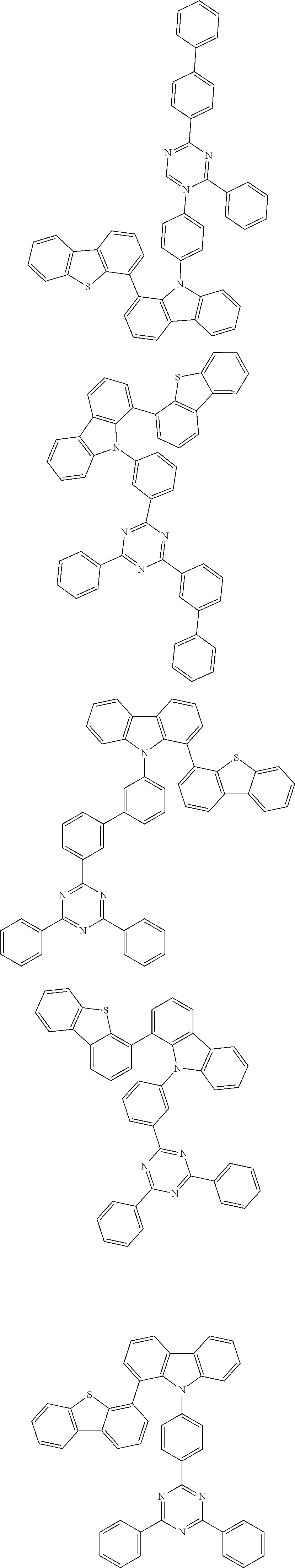

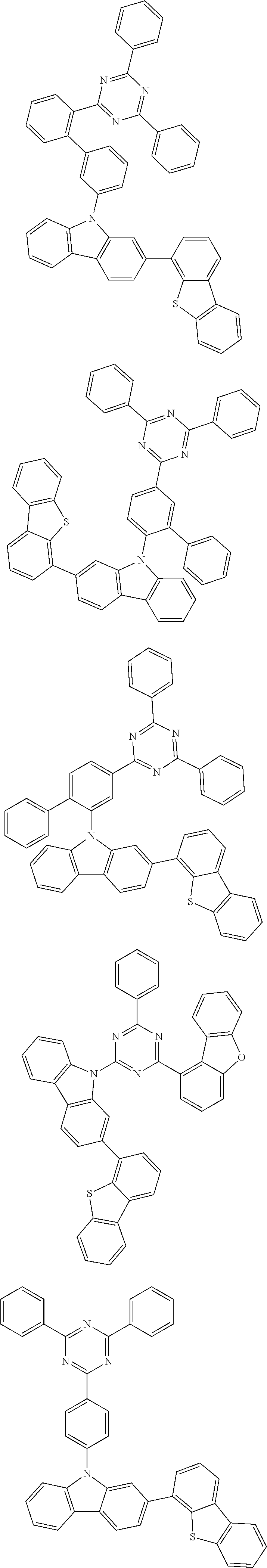

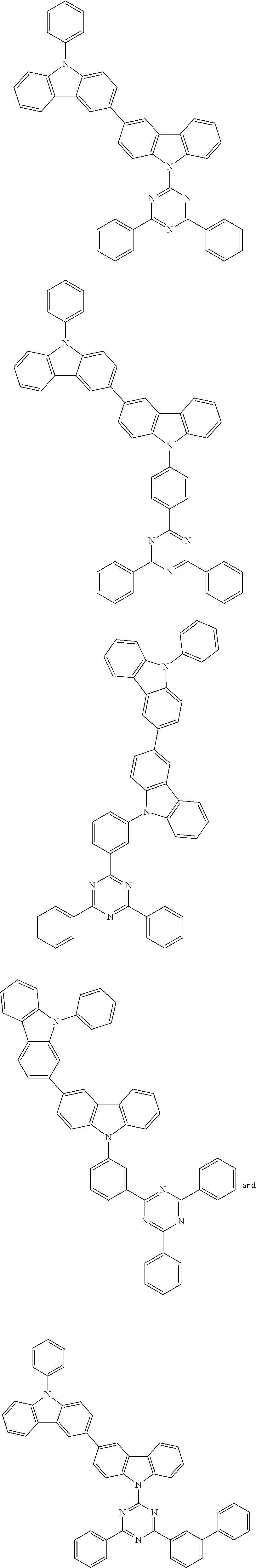

10. The organic electroluminescent material according to claim 8, wherein the compound represented by formula 12 is selected from the group consisting of the following compounds:

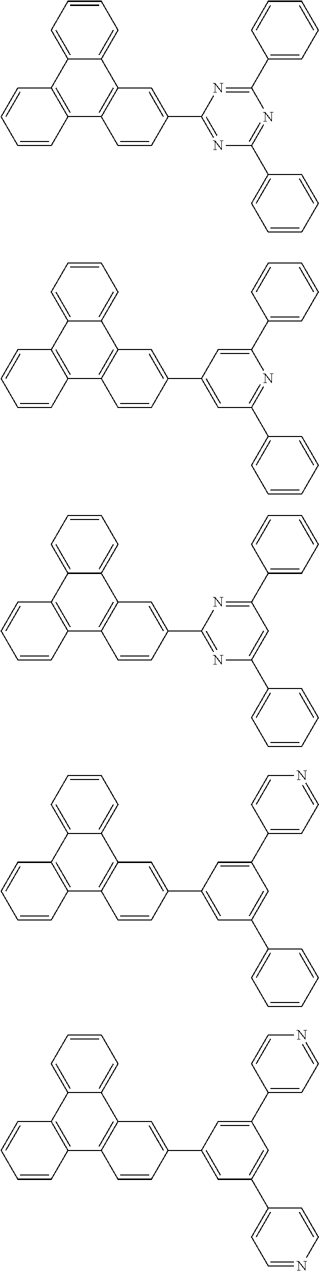

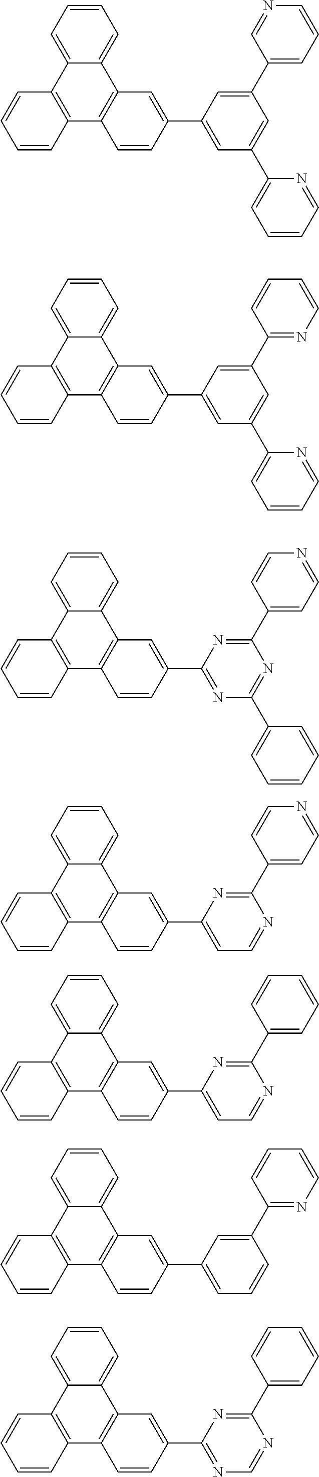

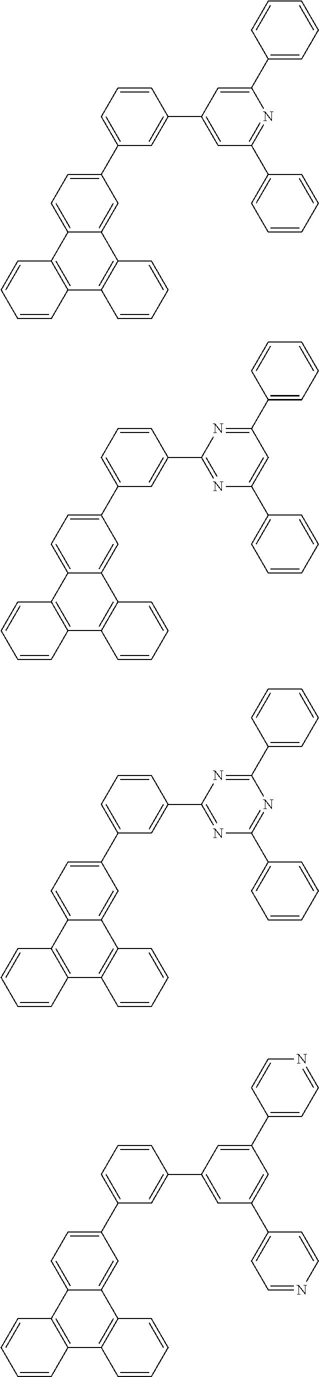 [SMILES: c1ccc(-c2cc(-c3cccc(-c4ccc5c6ccccc6c6ccccc6c5c4)c3)cc(-c3ccccc3)n2)cc1 ; c1ccc(-c2cc(-c3ccccc3)nc(-c3cccc(-c4ccc5c6ccccc6c6ccccc6c5c4)c3)n2)cc1 ; c1ccc(-c2nc(-c3ccccc3)nc(-c3cccc(-c4ccc5c6ccccc6c6ccccc6c5c4)c3)n2)cc1 ; c1cc(-c2cc(-c3ccncc3)cc(-c3ccncc3)c2)cc(-c2ccc3c4ccccc4c4ccccc4c3c2)c1]

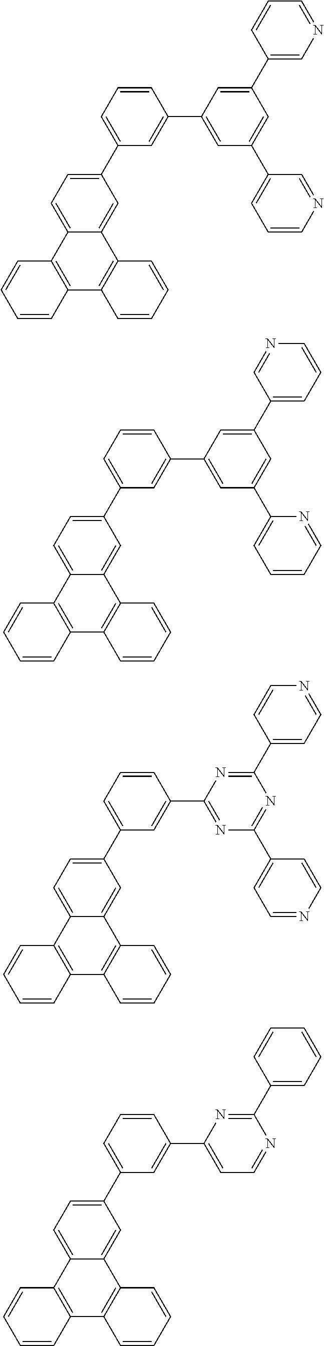 [SMILES: c1cncc(-c2cc(-c3cccnc3)cc(-c3cccc(-c4ccc5c6ccccc6c6ccccc6c5c4)c3)c2)c1 ; c1ccc(-c2cc(-c3cccnc3)cc(-c3cccc(-c4ccc5c6ccccc6c6ccccc6c5c4)c3)c2)nc1 ; c1cc(-c2ccc3c4ccccc4c4ccccc4c3c2)cc(-c2nc(-c3ccncc3)nc(-c3ccncc3)n2)c1 ; c1ccc(-c2nccc(-c3cccc(-c4ccc5c6ccccc6c6ccccc6c5c4)c3)n2)cc1]

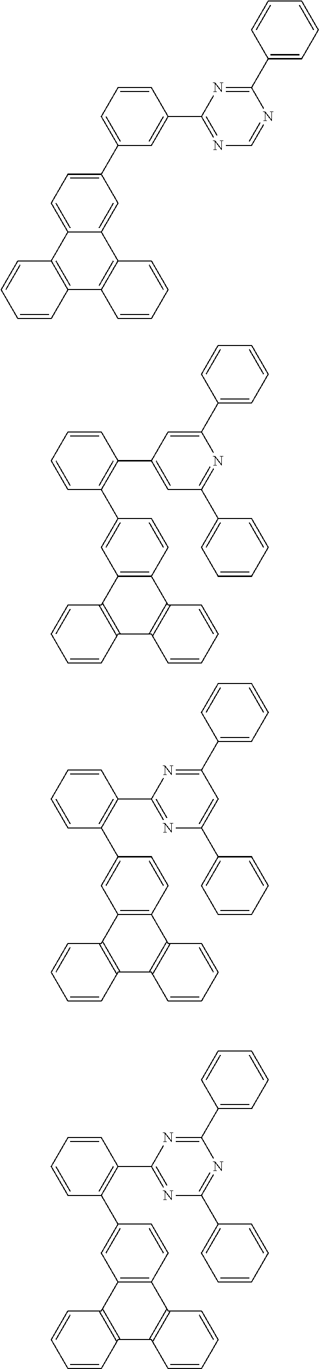

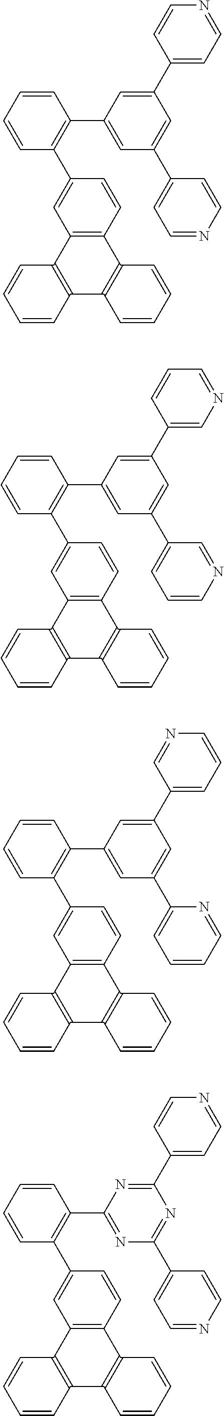 [SMILES: c1ccc(-c2ccc3c4ccccc4c4ccccc4c3c2)c(-c2cc(-c3ccncc3)cc(-c3ccncc3)c2)c1 ; c1cncc(-c2cc(-c3cccnc3)cc(-c3ccccc3-c3ccc4c5ccccc5c5ccccc5c4c3)c2)c1 ; c1ccc(-c2cc(-c3cccnc3)cc(-c3ccccc3-c3ccc4c5ccccc5c5ccccc5c4c3)c2)nc1 ; c1ccc(-c2nc(-c3ccncc3)nc(-c3ccncc3)n2)c(-c2ccc3c4ccccc4c4ccccc4c3c2)c1]

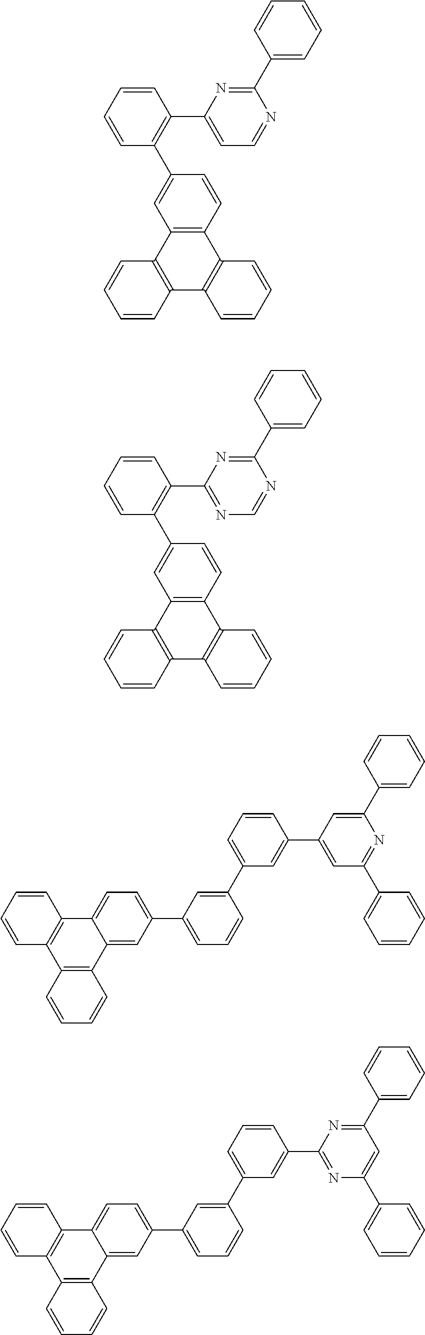 [SMILES: c1ccc(-c2nccc(-c3ccccc3-c3ccc4c5ccccc5c5ccccc5c4c3)n2)cc1 ; c1ccc(-c2ncnc(-c3ccccc3-c3ccc4c5ccccc5c5ccccc5c4c3)n2)cc1 ; c1ccc(-c2cc(-c3cccc(-c4cccc(-c5ccc6c7ccccc7c7ccccc7c6c5)c4)c3)cc(-c3ccccc3)n2)cc1 ; c1ccc(-c2cc(-c3ccccc3)nc(-c3cccc(-c4cccc(-c5ccc6c7ccccc7c7ccccc7c6c5)c4)c3)n2)cc1]

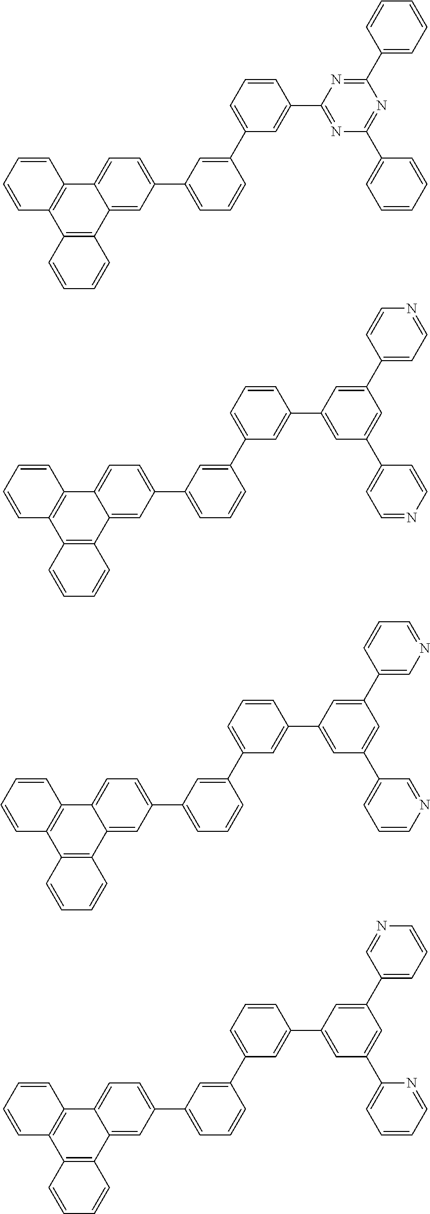 [SMILES: c1ccc(-c2nc(-c3ccccc3)nc(-c3cccc(-c4cccc(-c5ccc6c7ccccc7c7ccccc7c6c5)c4)c3)n2)cc1 ; c1cc(-c2cccc(-c3ccc4c5ccccc5c5ccccc5c4c3)c2)cc(-c2cc(-c3ccncc3)cc(-c3ccncc3)c2)c1 ; c1cncc(-c2cc(-c3cccnc3)cc(-c3cccc(-c4cccc(-c5ccc6c7ccccc7c7ccccc7c6c5)c4)c3)c2)c1 ; c1ccc(-c2cc(-c3cccnc3)cc(-c3cccc(-c4cccc(-c5ccc6c7ccccc7c7ccccc7c6c5)c4)c3)c2)nc1]

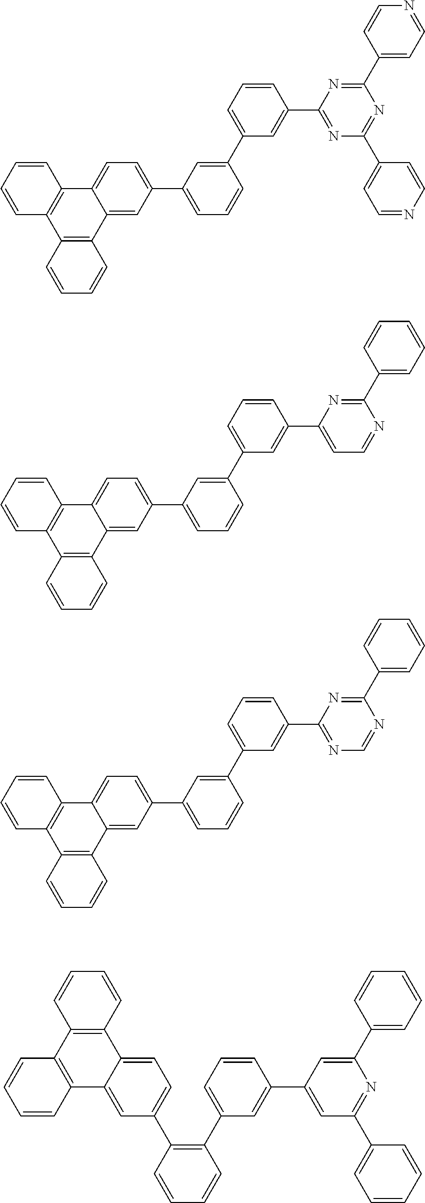 [SMILES: c1cc(-c2cccc(-c3nc(-c4ccncc4)nc(-c4ccncc4)n3)c2)cc(-c2ccc3c4ccccc4c4ccccc4c3c2)c1 ; c1ccc(-c2nccc(-c3cccc(-c4cccc(-c5ccc6c7ccccc7c7ccccc7c6c5)c4)c3)n2)cc1 ; c1ccc(-c2ncnc(-c3cccc(-c4cccc(-c5ccc6c7ccccc7c7ccccc7c6c5)c4)c3)n2)cc1 ; c1ccc(-c2cc(-c3cccc(-c4ccccc4-c4ccc5c6ccccc6c6ccccc6c5c4)c3)cc(-c3ccccc3)n2)cc1]

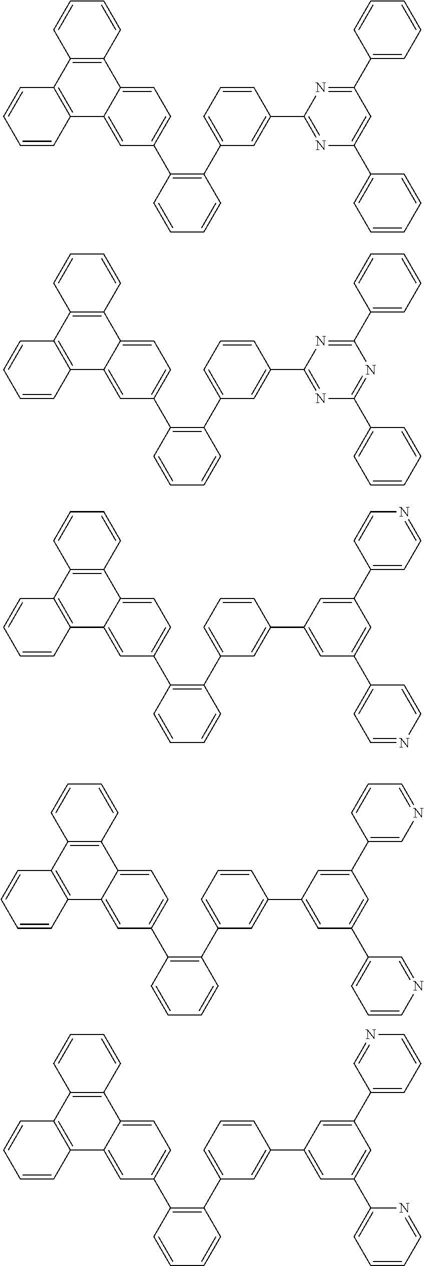

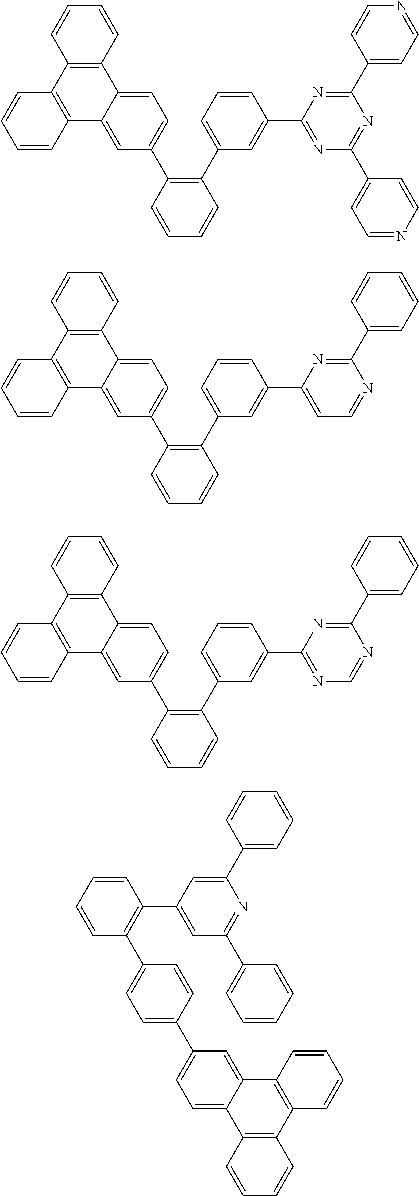

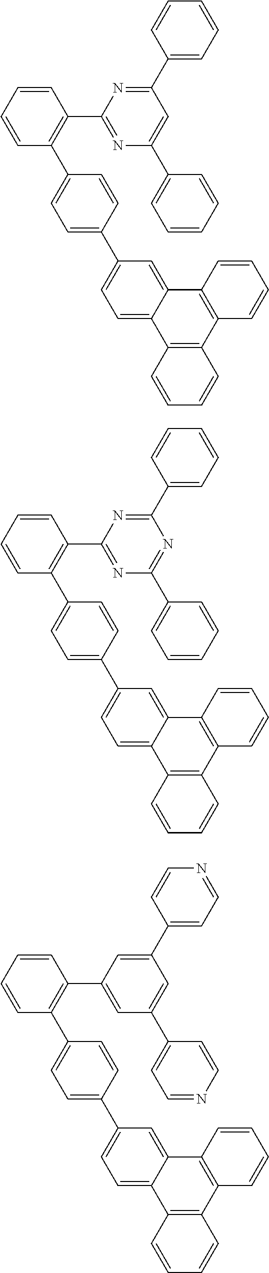

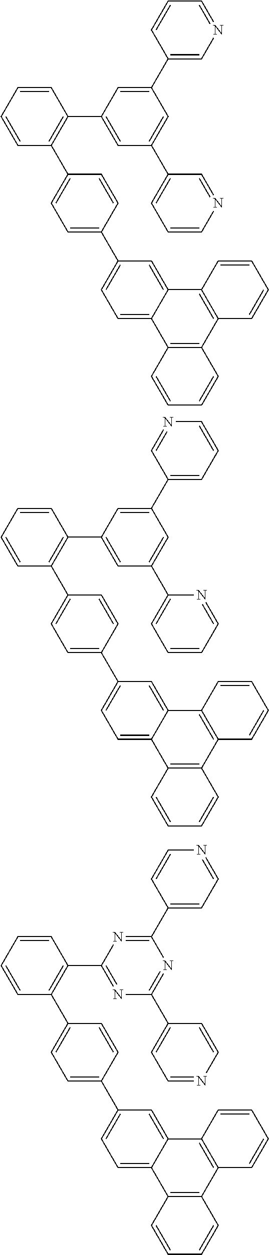

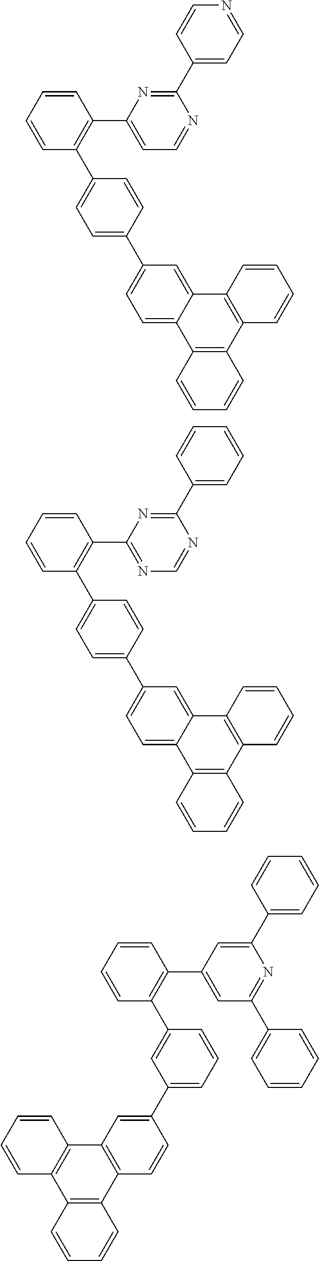

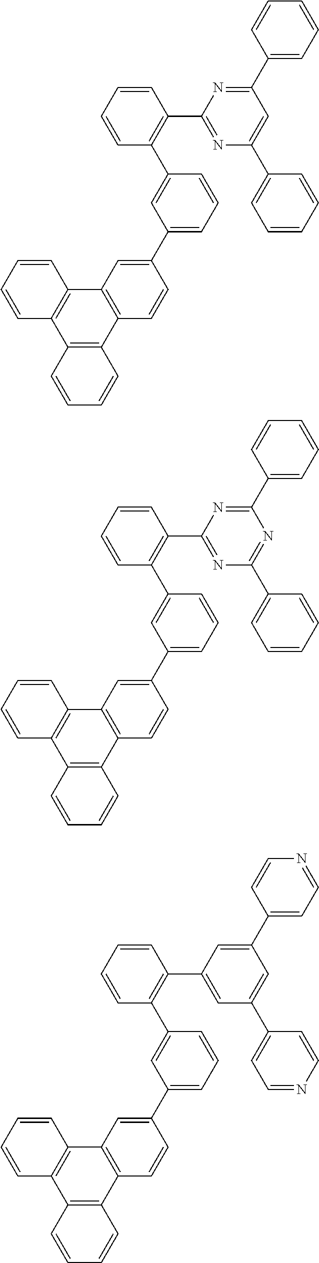 [SMILES: c1ccc(-c2cc(-c3ccccc3)nc(-c3ccccc3-c3cccc(-c4ccc5c6ccccc6c6ccccc6c5c4)c3)n2)cc1 ; c1ccc(-c2nc(-c3ccccc3)nc(-c3ccccc3-c3cccc(-c4ccc5c6ccccc6c6ccccc6c5c4)c3)n2)cc1 ; c1cc(-c2ccc3c4ccccc4c4ccccc4c3c2)cc(-c2ccccc2-c2cc(-c3ccncc3)cc(-c3ccncc3)c2)c1]

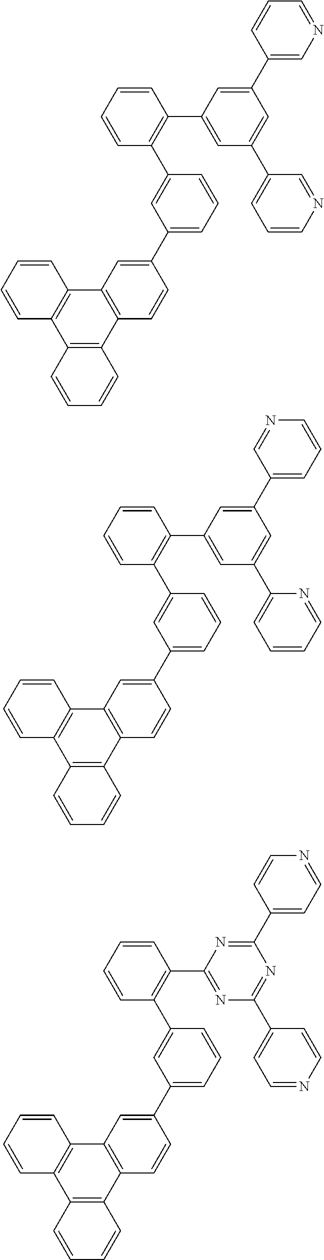 [SMILES: c1cncc(-c2cc(-c3cccnc3)cc(-c3ccccc3-c3cccc(-c4ccc5c6ccccc6c6ccccc6c5c4)c3)c2)c1 ; c1ccc(-c2cc(-c3cccnc3)cc(-c3ccccc3-c3cccc(-c4ccc5c6ccccc6c6ccccc6c5c4)c3)c2)nc1 ; c1cc(-c2ccc3c4ccccc4c4ccccc4c3c2)cc(-c2ccccc2-c2nc(-c3ccncc3)nc(-c3ccncc3)n2)c1]

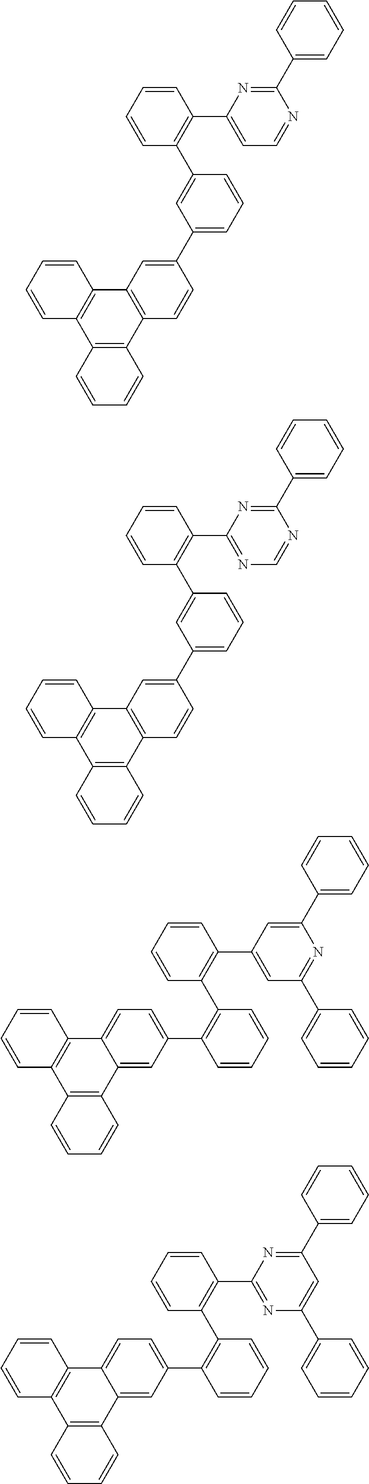 [SMILES: c1ccc(-c2nccc(-c3ccccc3-c3cccc(-c4ccc5c6ccccc6c6ccccc6c5c4)c3)n2)cc1 ; c1ccc(-c2ncnc(-c3ccccc3-c3cccc(-c4ccc5c6ccccc6c6ccccc6c5c4)c3)n2)cc1 ; c1ccc(-c2cc(-c3ccccc3-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccccc3-c3ccc4c5ccccc5c5ccccc5c4c3)n2)cc1]

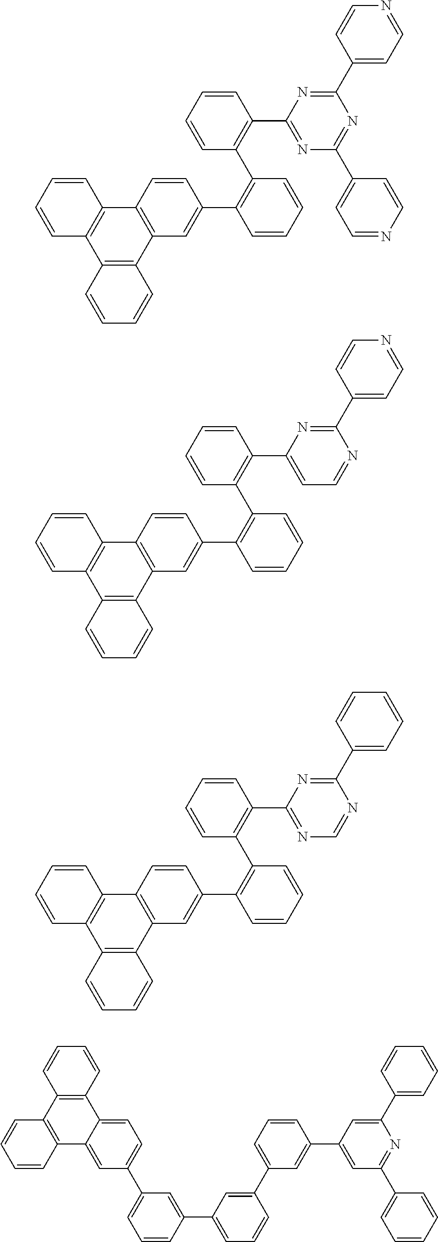

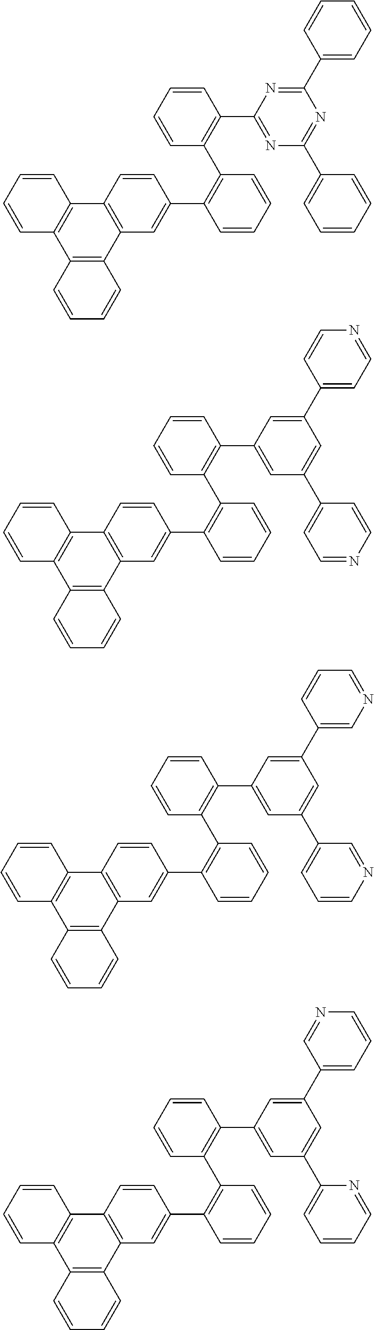 [SMILES: c1ccc(-c2nc(-c3ccccc3)nc(-c3ccccc3-c3ccccc3-c3ccc4c5ccccc5c5ccccc5c4c3)n2)cc1 ; c1ccc(-c2ccccc2-c2ccc3c4ccccc4c4ccccc4c3c2)c(-c2cc(-c3ccncc3)cc(-c3ccncc3)c2)c1 ; c1cncc(-c2cc(-c3cccnc3)cc(-c3ccccc3-c3ccccc3-c3ccc4c5ccccc5c5ccccc5c4c3)c2)c1 ; c1ccc(-c2cc(-c3cccnc3)cc(-c3ccccc3-c3ccccc3-c3ccc4c5ccccc5c5ccccc5c4c3)c2)nc1]

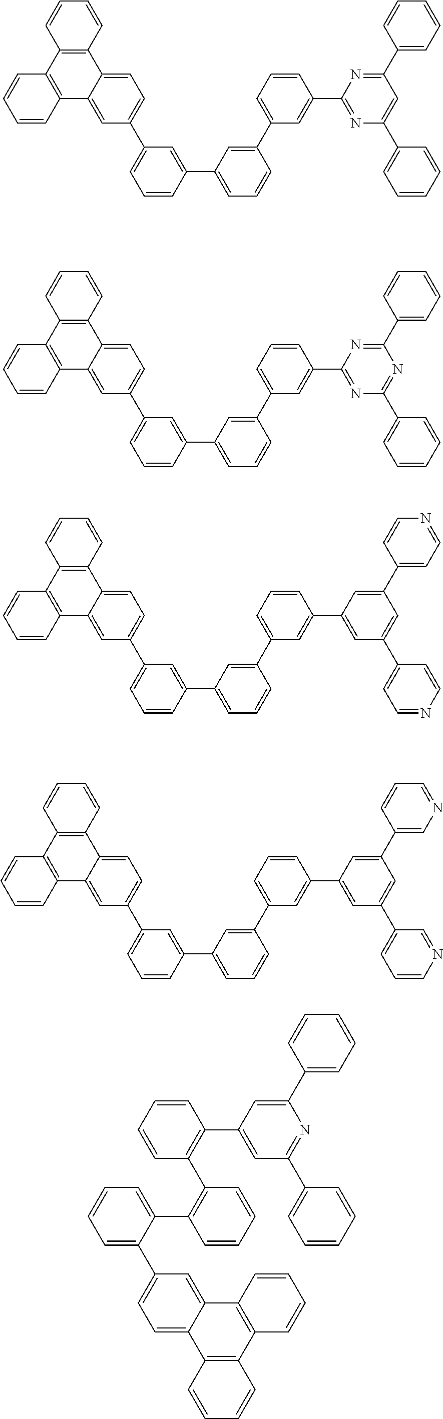 [SMILES: c1ccc(-c2cc(-c3ccccc3)nc(-c3cccc(-c4cccc(-c5cccc(-c6ccc7c8ccccc8c8ccccc8c7c6)c5)c4)c3)n2)cc1 ; c1ccc(-c2nc(-c3ccccc3)nc(-c3cccc(-c4cccc(-c5cccc(-c6ccc7c8ccccc8c8ccccc8c7c6)c5)c4)c3)n2)cc1 ; c1cc(-c2cccc(-c3cc(-c4ccncc4)cc(-c4ccncc4)c3)c2)cc(-c2cccc(-c3ccc4c5ccccc5c5ccccc5c4c3)c2)c1 ; c1cncc(-c2cc(-c3cccnc3)cc(-c3cccc(-c4cccc(-c5cccc(-c6ccc7c8ccccc8c8ccccc8c7c6)c5)c4)c3)c2)c1 ; c1ccc(-c2cc(-c3ccccc3-c3ccccc3-c3ccccc3-c3ccc4c5ccccc5c5ccccc5c4c3)cc(-c3ccccc3)n2)cc1]

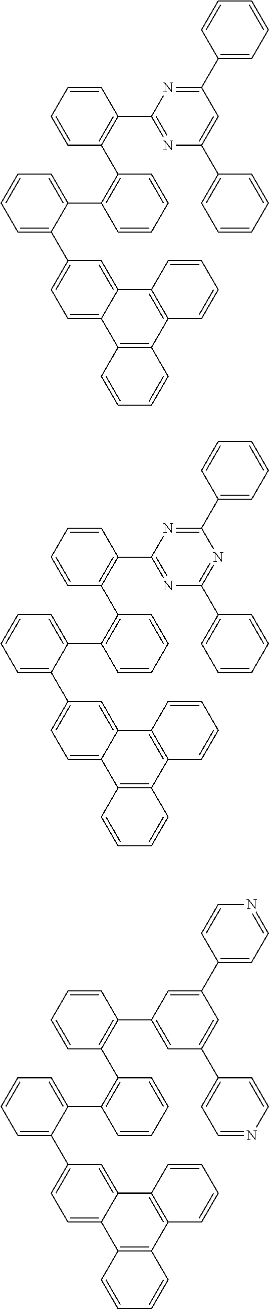 [SMILES: c1ccc(-c2cc(-c3ccccc3)nc(-c3ccccc3-c3ccccc3-c3ccccc3-c3ccc4c5ccccc5c5ccccc5c4c3)n2)cc1 ; c1ccc(-c2nc(-c3ccccc3)nc(-c3ccccc3-c3ccccc3-c3ccccc3-c3ccc4c5ccccc5c5ccccc5c4c3)n2)cc1 ; c1ccc(-c2ccccc2-c2ccccc2-c2ccc3c4ccccc4c4ccccc4c3c2)c(-c2cc(-c3ccncc3)cc(-c3ccncc3)c2)c1]

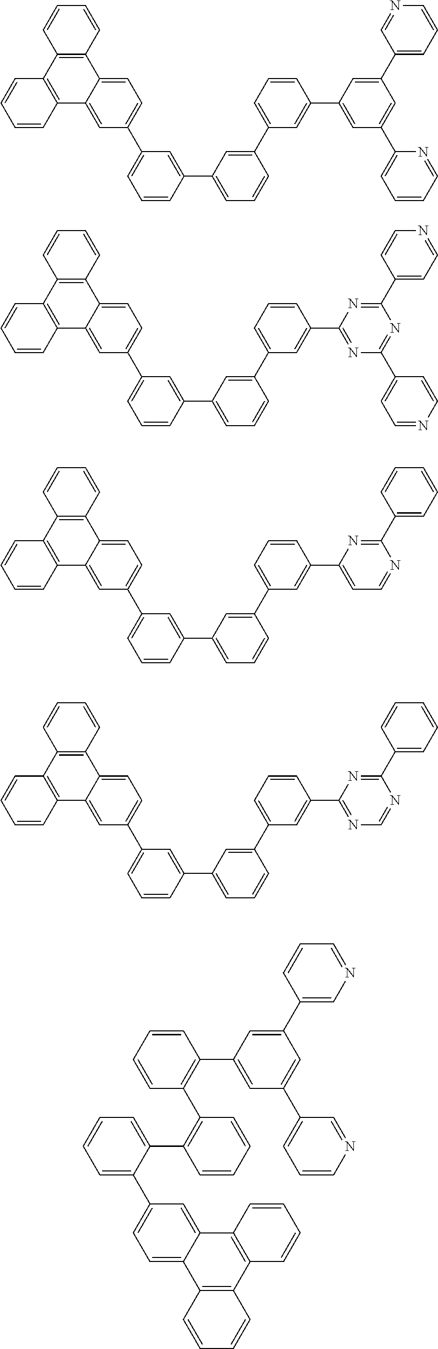

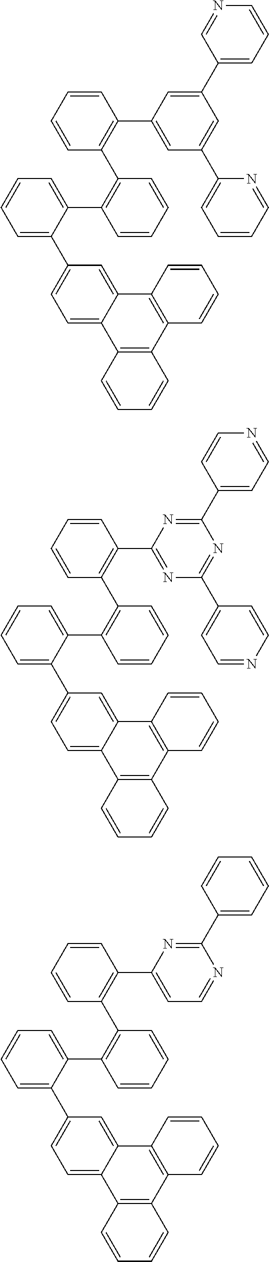 [SMILES: c1ccc(-c2cc(-c3cccnc3)cc(-c3ccccc3-c3ccccc3-c3ccccc3-c3ccc4c5ccccc5c5ccccc5c4c3)c2)nc1 ; c1ccc(-c2ccccc2-c2ccccc2-c2nc(-c3ccncc3)nc(-c3ccncc3)n2)c(-c2ccc3c4ccccc4c4ccccc4c3c2)c1 ; c1ccc(-c2nccc(-c3ccccc3-c3ccccc3-c3ccccc3-c3ccc4c5ccccc5c5ccccc5c4c3)n2)cc1]

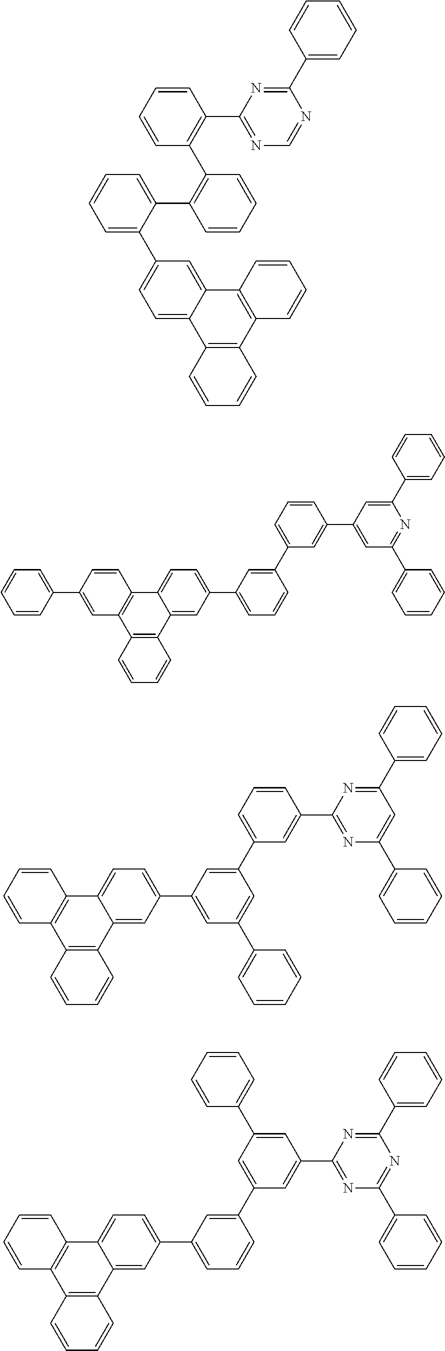 [SMILES: c1ccc(-c2ncnc(-c3ccccc3-c3ccccc3-c3ccccc3-c3ccc4c5ccccc5c5ccccc5c4c3)n2)cc1 ; c1ccc(-c2ccc3c4ccc(-c5cccc(-c6cccc(-c7cc(-c8ccccc8)nc(-c8ccccc8)c7)c6)c5)cc4c4ccccc4c3c2)cc1 ; c1ccc(-c2cc(-c3cccc(-c4nc(-c5ccccc5)cc(-c5ccccc5)n4)c3)cc(-c3ccc4c5ccccc5c5ccccc5c4c3)c2)cc1 ; c1ccc(-c2cc(-c3cccc(-c4ccc5c6ccccc6c6ccccc6c5c4)c3)cc(-c3nc(-c4ccccc4)nc(-c4ccccc4)n3)c2)cc1]

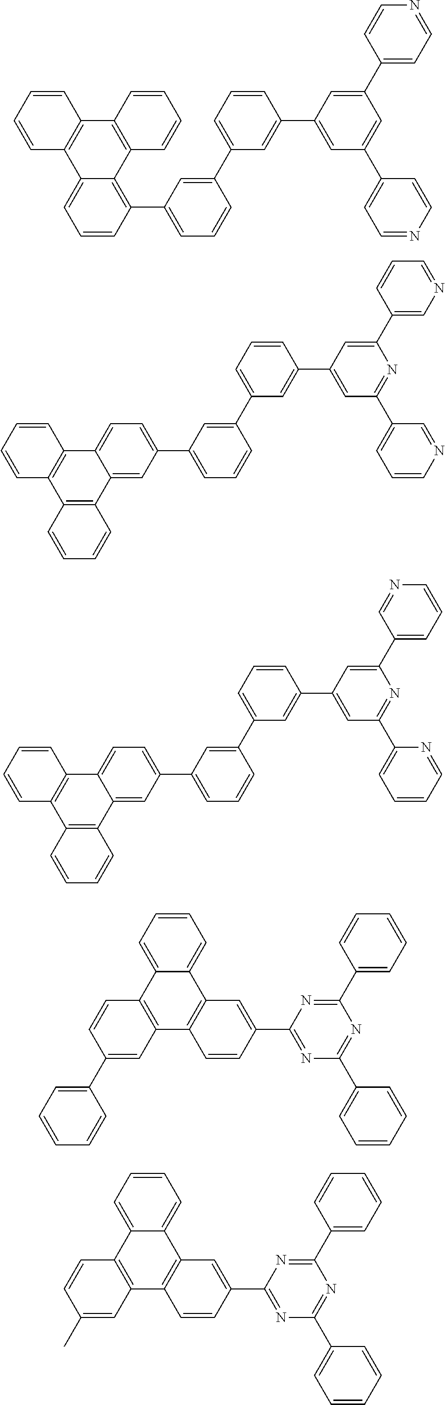

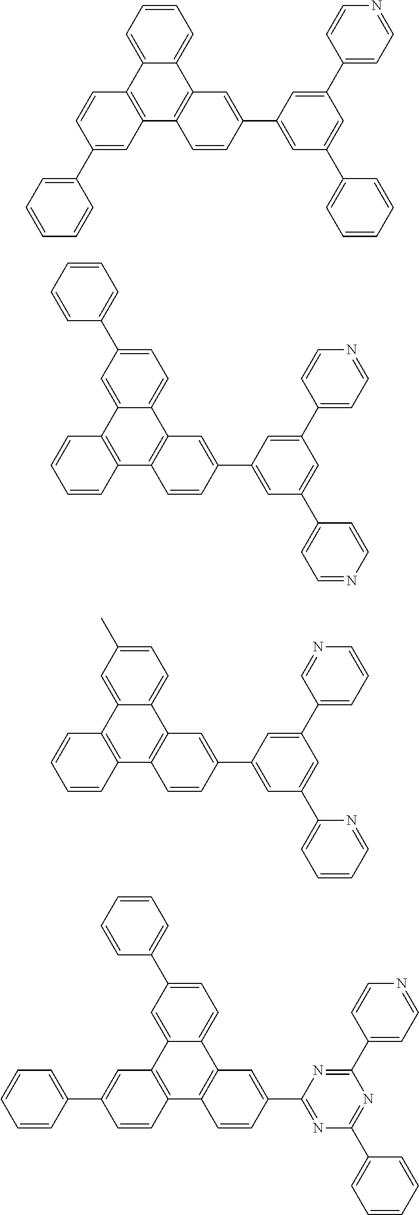

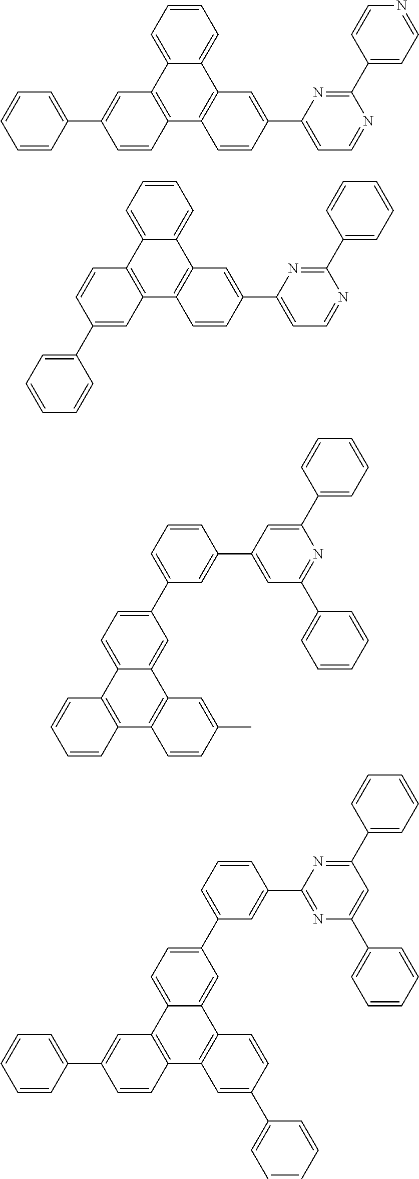

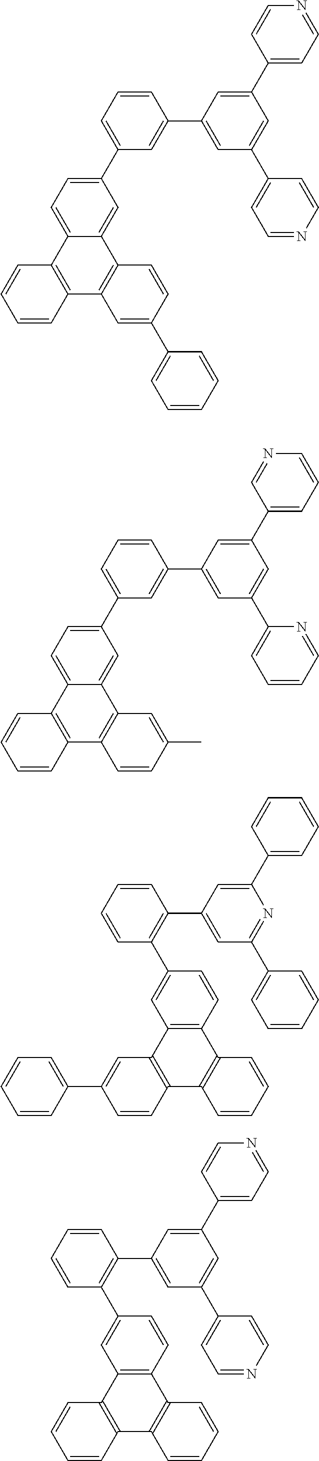

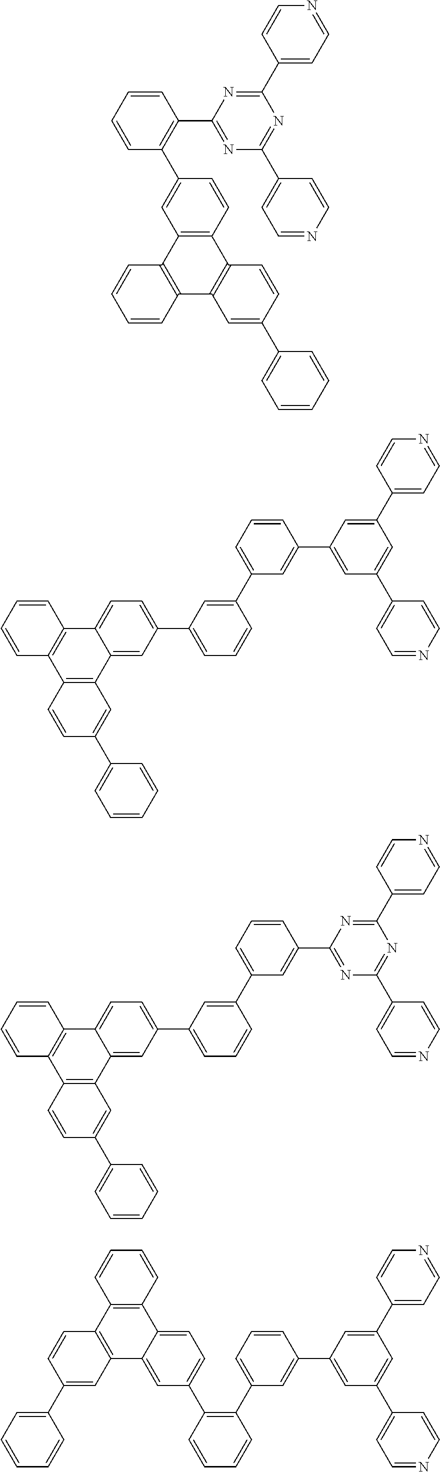

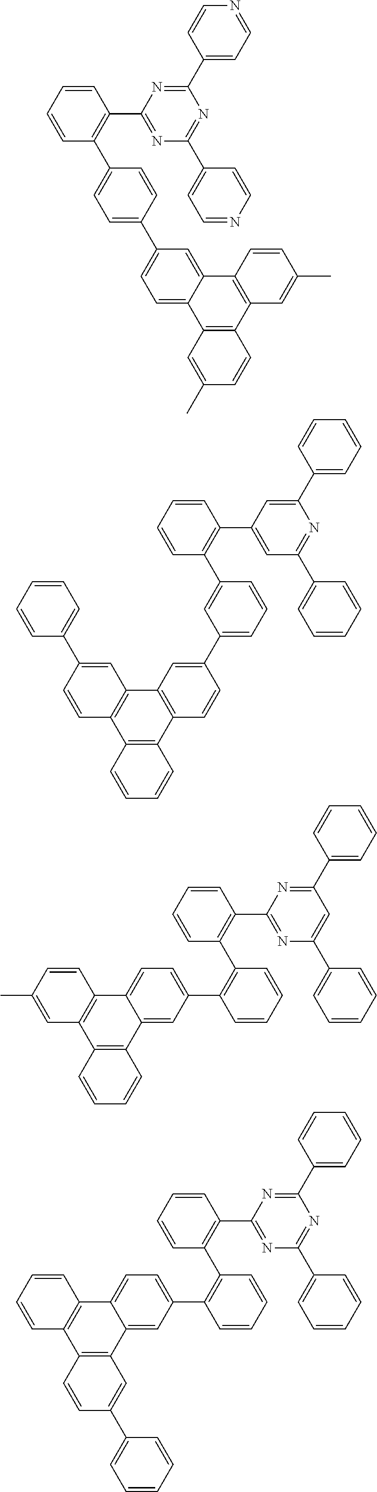

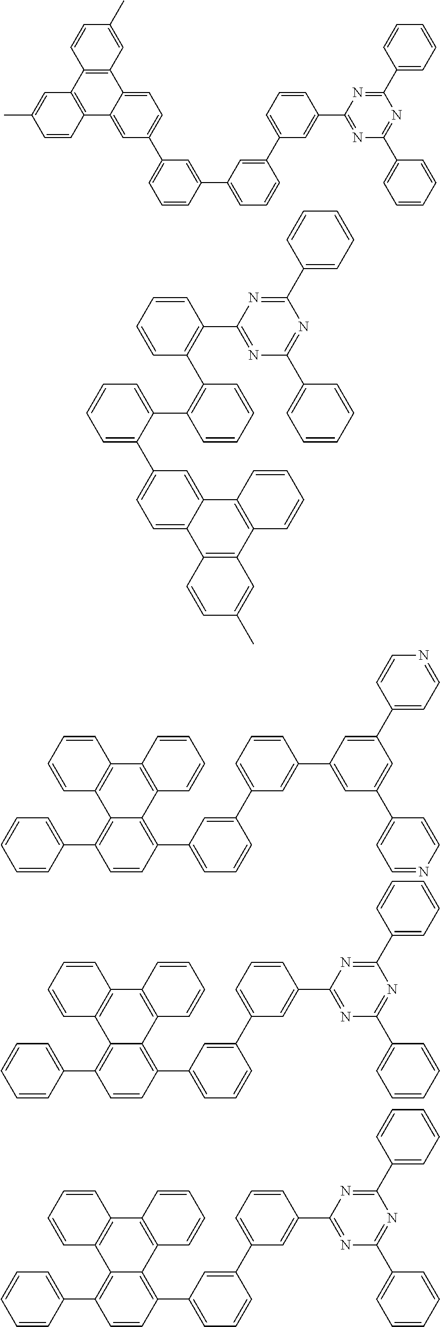

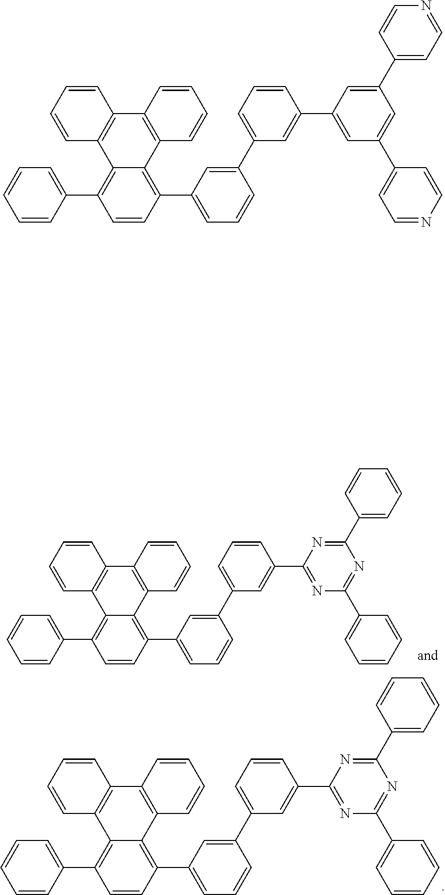

11. The organic electroluminescent material according to claim 8, wherein the compound represented by formula 13 or 14 is selected from the group consisting of the following compounds:

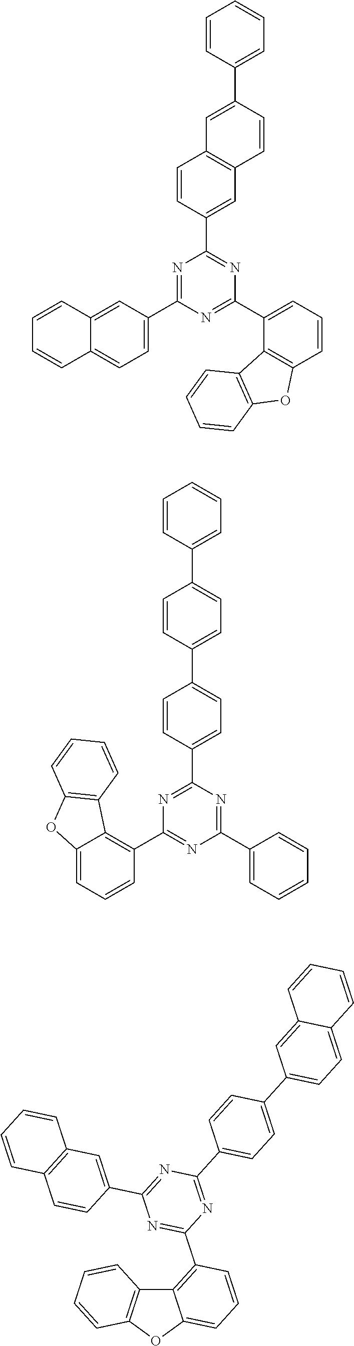 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccccc5)nc(-c5cccc6oc7ccccc7c56)n4)cc3)cc2)cc1 ; c1ccc2cc(-c3ccc(-c4nc(-c5ccc6ccccc6c5)nc(-c5cccc6oc7ccccc7c56)n4)cc3)ccc2c1]

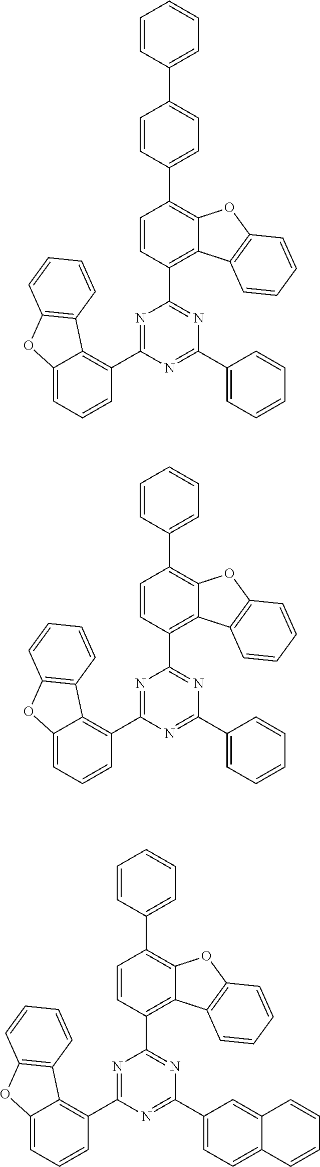 [SMILES: c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2nc(-c3cccc4oc5ccccc5c34)nc(-c3ccc(-c4ccccc4)c4oc5ccccc5c34)n2)cc1 ; c1ccc(-c2ccc(-c3nc(-c4ccc5ccccc5c4)nc(-c4cccc5oc6ccccc6c45)n3)c3c2oc2ccccc23)cc1]

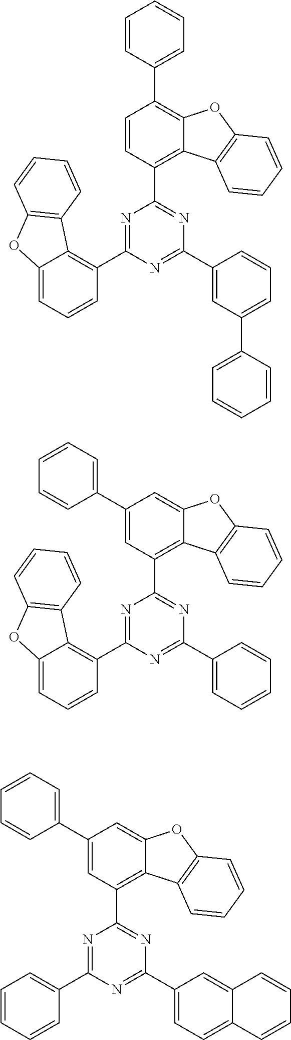 [SMILES: c1ccc(-c2cccc(-c3nc(-c4cccc5oc6ccccc6c45)nc(-c4ccc(-c5ccccc5)c5oc6ccccc6c45)n3)c2)cc1 ; c1ccc(-c2cc(-c3nc(-c4ccccc4)nc(-c4cccc5oc6ccccc6c45)n3)c3c(c2)oc2ccccc23)cc1 ; c1ccc(-c2cc(-c3nc(-c4ccccc4)nc(-c4ccc5ccccc5c4)n3)c3c(c2)oc2ccccc23)cc1]

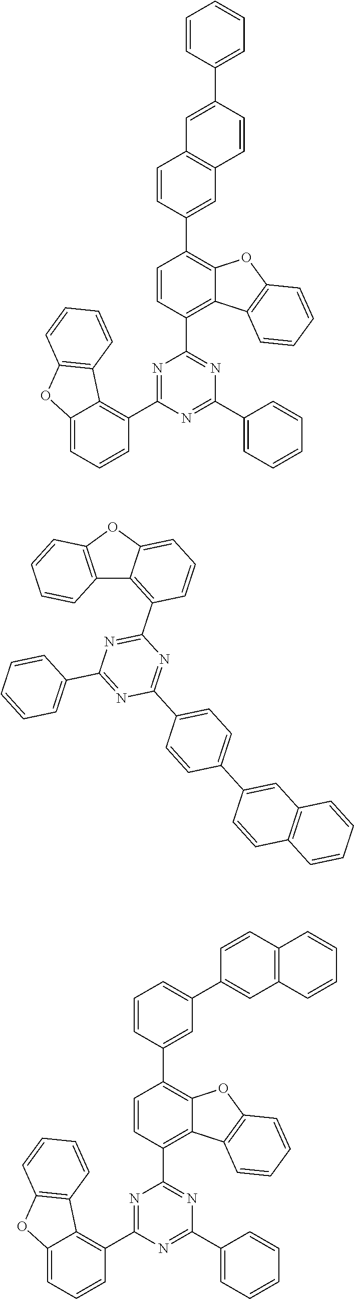 [SMILES: c1ccc(-c2ccc3cc(-c4ccc(-c5nc(-c6ccccc6)nc(-c6cccc7oc8ccccc8c67)n5)c5c4oc4ccccc45)ccc3c2)cc1 ; c1ccc(-c2nc(-c3ccc(-c4ccc5ccccc5c4)cc3)nc(-c3cccc4oc5ccccc5c34)n2)cc1 ; c1ccc(-c2nc(-c3cccc4oc5ccccc5c34)nc(-c3ccc(-c4cccc(-c5ccc6ccccc6c5)c4)c4oc5ccccc5c34)n2)cc1]

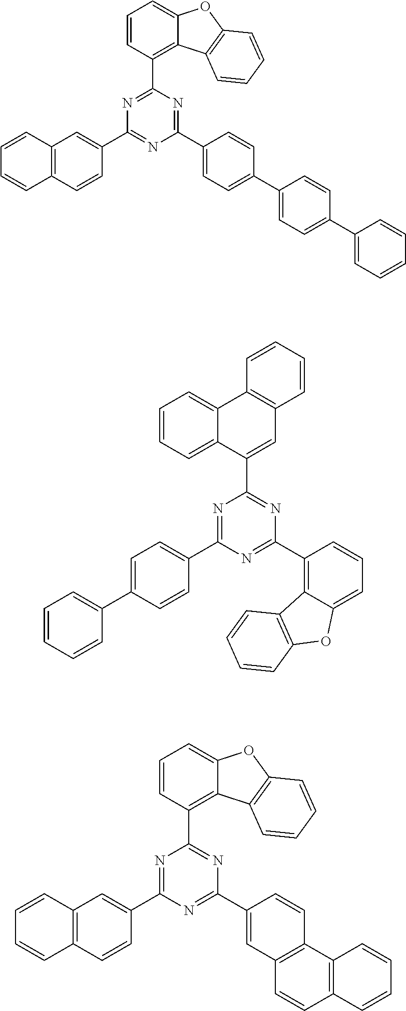 [SMILES: c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3nc(-c4cc5ccccc5c5ccccc45)nc(-c4cccc5oc6ccccc6c45)n3)cc2)cc1 ; c1ccc2cc(-c3nc(-c4ccc5c(ccc6ccccc65)c4)nc(-c4cccc5oc6ccccc6c45)n3)ccc2c1]

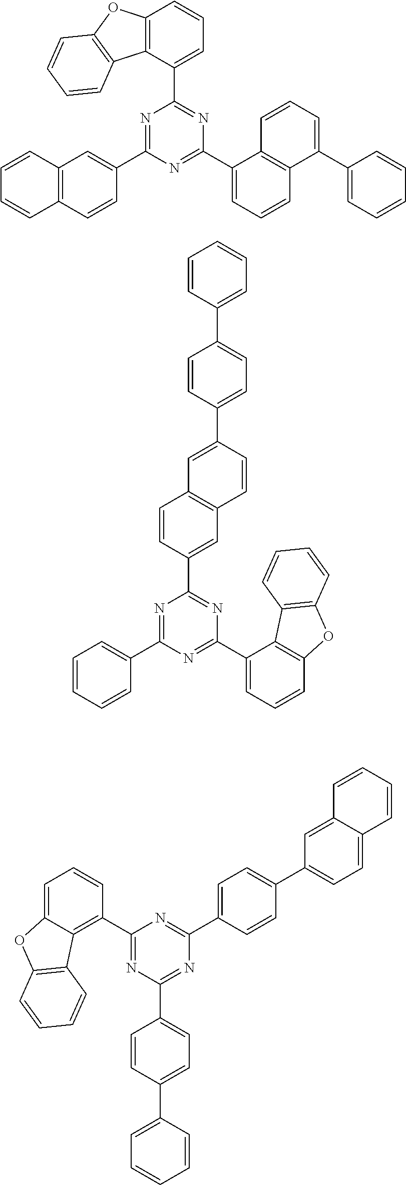

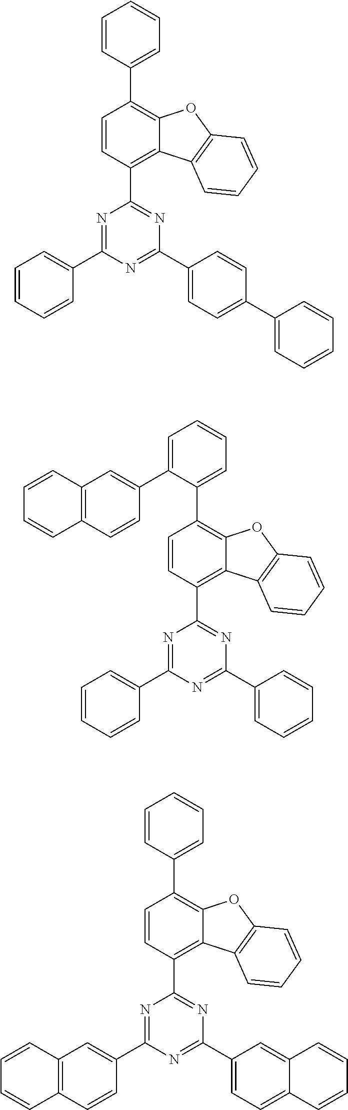 [SMILES: c1ccc(-c2ccc(-c3nc(-c4ccccc4)nc(-c4ccc(-c5ccccc5)c5oc6ccccc6c45)n3)cc2)cc1 ; c1ccc(-c2nc(-c3ccccc3)nc(-c3ccc(-c4ccccc4-c4ccc5ccccc5c4)c4oc5ccccc5c34)n2)cc1 ; c1ccc(-c2ccc(-c3nc(-c4ccc5ccccc5c4)nc(-c4ccc5ccccc5c4)n3)c3c2oc2ccccc23)cc1]

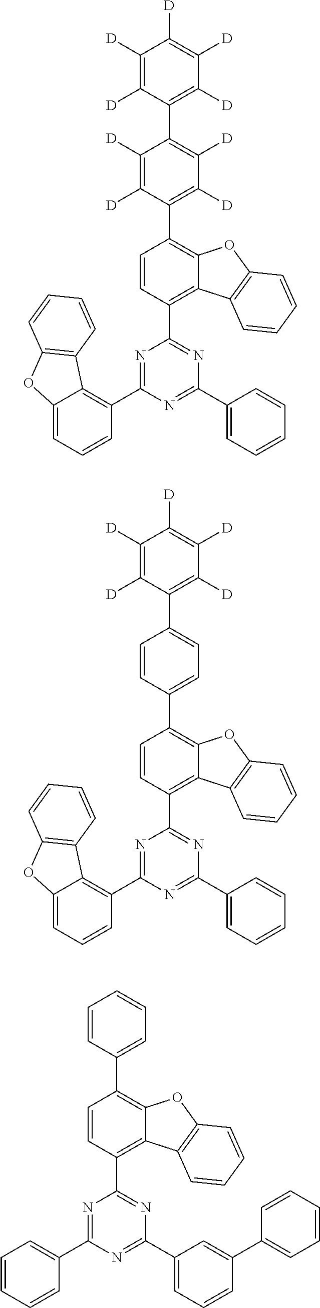 [SMILES: [2H]c1c([2H])c([2H])c(-c2c([2H])c([2H])c(-c3ccc(-c4nc(-c5ccccc5)nc(-c5cccc6oc7ccccc7c56)n4)c4c3oc3ccccc34)c([2H])c2[2H])c([2H])c1[2H] ; [2H]c1c([2H])c([2H])c(-c2ccc(-c3ccc(-c4nc(-c5ccccc5)nc(-c5cccc6oc7ccccc7c56)n4)c4c3oc3ccccc34)cc2)c([2H])c1[2H] ; c1ccc(-c2cccc(-c3nc(-c4ccccc4)nc(-c4ccc(-c5ccccc5)c5oc6ccccc6c45)n3)c2)cc1]

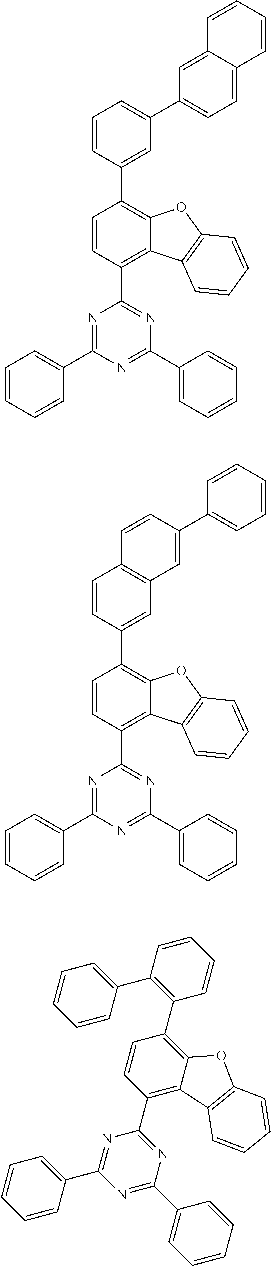 [SMILES: c1ccc(-c2nc(-c3ccccc3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccccc6)n5)c5c4oc4ccccc45)cc3c2)cc1 ; c1ccc(-c2nc(-c3ccccc3)nc(-c3ccc(-c4ccccc4-c4ccccc4)c4oc5ccccc5c34)n2)cc1]

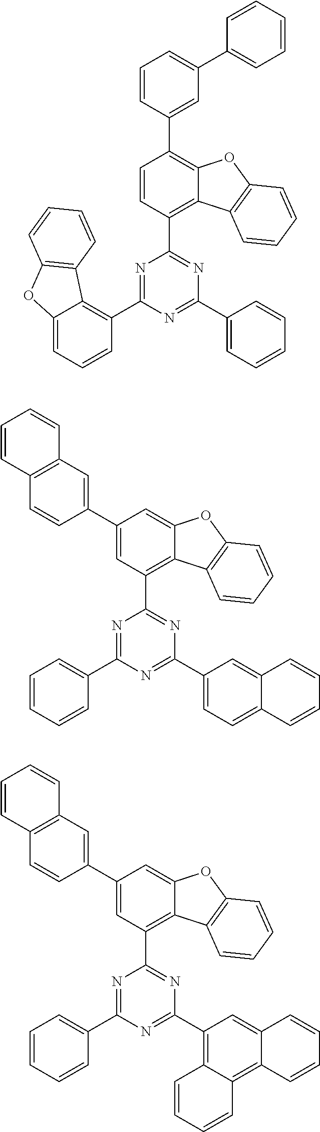 [SMILES: c1ccc(-c2cccc(-c3ccc(-c4nc(-c5ccccc5)nc(-c5cccc6oc7ccccc7c56)n4)c4c3oc3ccccc34)c2)cc1 ; c1ccc(-c2nc(-c3ccc4ccccc4c3)nc(-c3cc(-c4ccc5ccccc5c4)cc4oc5ccccc5c34)n2)cc1 ; c1ccc(-c2nc(-c3cc4ccccc4c4ccccc34)nc(-c3cc(-c4ccc5ccccc5c4)cc4oc5ccccc5c34)n2)cc1]

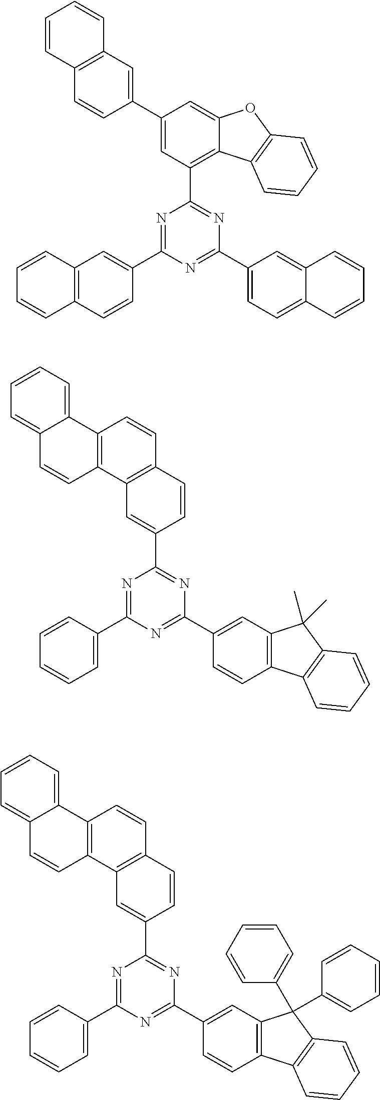 [SMILES: c1ccc2cc(-c3cc(-c4nc(-c5ccc6ccccc6c5)nc(-c5ccc6ccccc6c5)n4)c4c(c3)oc3ccccc34)ccc2c1 ; CC1(C)c2ccccc2-c2ccc(-c3nc(-c4ccccc4)nc(-c4ccc5ccc6c7ccccc7ccc6c5c4)n3)cc21 ; c1ccc(-c2nc(-c3ccc4c(c3)C(c3ccccc3)(c3ccccc3)c3ccccc3-4)nc(-c3ccc4ccc5c6ccccc6ccc5c4c3)n2)cc1]

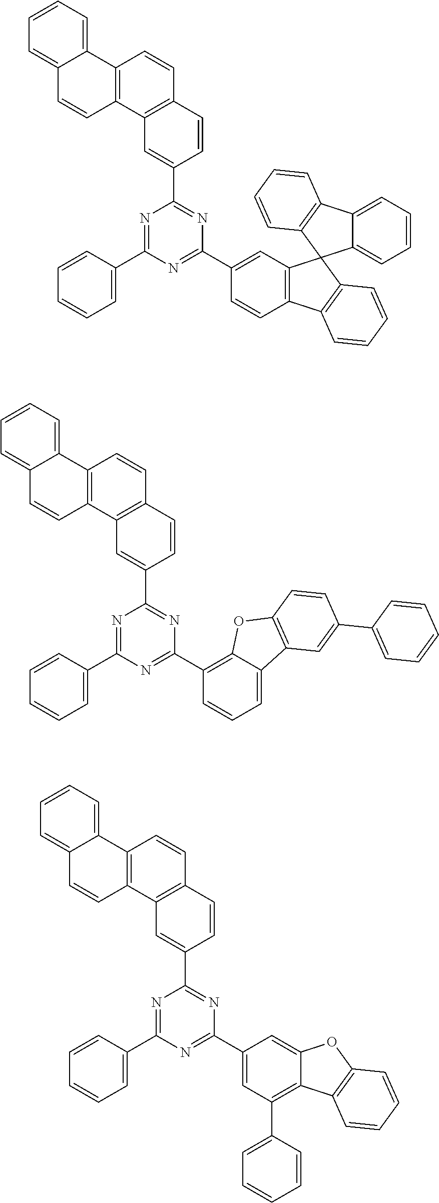 [SMILES: c1ccc(-c2nc(-c3ccc4c(c3)C3(c5ccccc5-c5ccccc53)c3ccccc3-4)nc(-c3ccc4ccc5c6ccccc6ccc5c4c3)n2)cc1 ; c1ccc(-c2ccc3oc4c(-c5nc(-c6ccccc6)nc(-c6ccc7ccc8c9ccccc9ccc8c7c6)n5)cccc4c3c2)cc1 ; c1ccc(-c2nc(-c3cc(-c4ccccc4)c4c(c3)oc3ccccc34)nc(-c3ccc4ccc5c6ccccc6ccc5c4c3)n2)cc1]

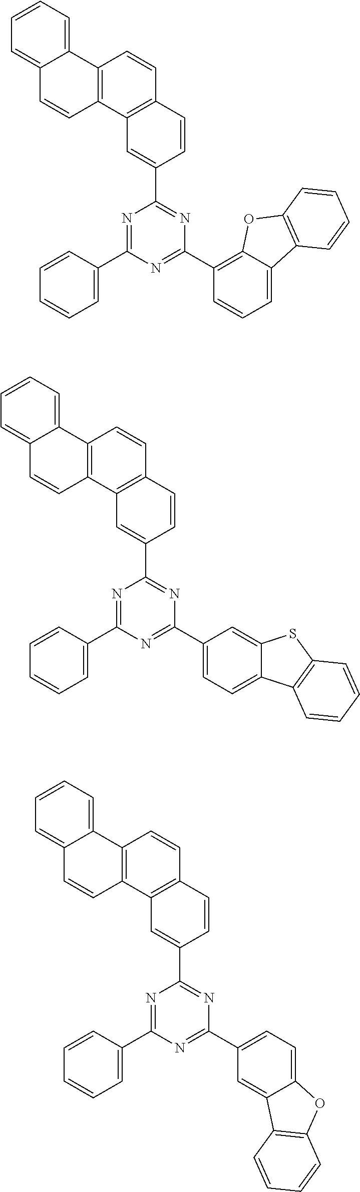 [SMILES: c1ccc(-c2nc(-c3ccc4ccc5c6ccccc6ccc5c4c3)nc(-c3cccc4c3oc3ccccc34)n2)cc1 ; c1ccc(-c2nc(-c3ccc4c(c3)sc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3ccc4oc5ccccc5c4c3)n2)cc1]

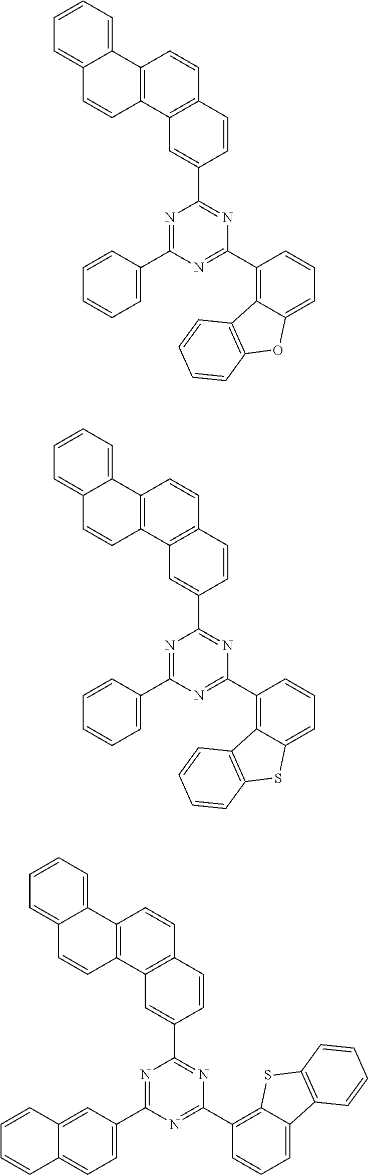 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1 ; c1ccc2cc(-c3nc(-c4ccc5ccc6c7ccccc7ccc6c5c4)nc(-c4cccc5c4sc4ccccc45)n3)ccc2c1]

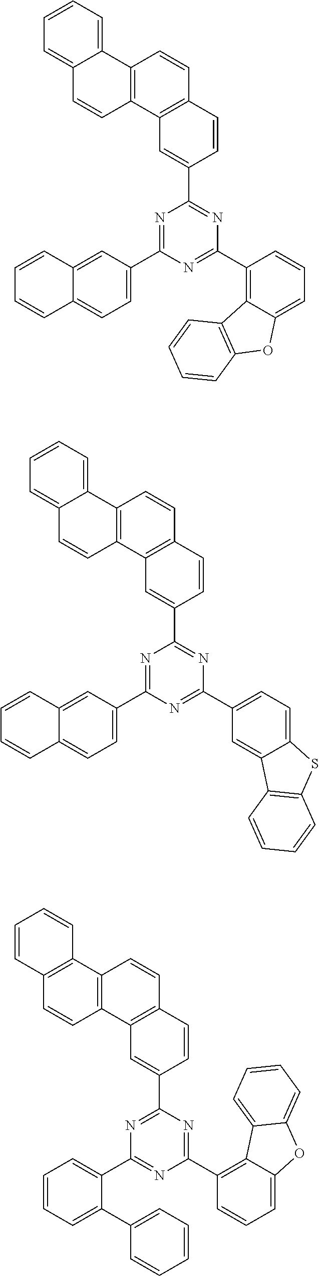 [SMILES: c1ccc2cc(-c3nc(-c4ccc5ccc6c7ccccc7ccc6c5c4)nc(-c4cccc5oc6ccccc6c45)n3)ccc2c1 ; c1ccc2cc(-c3nc(-c4ccc5ccc6c7ccccc7ccc6c5c4)nc(-c4ccc5sc6ccccc6c5c4)n3)ccc2c1 ; c1ccc(-c2ccccc2-c2nc(-c3ccc4ccc5c6ccccc6ccc5c4c3)nc(-c3cccc4oc5ccccc5c34)n2)cc1]

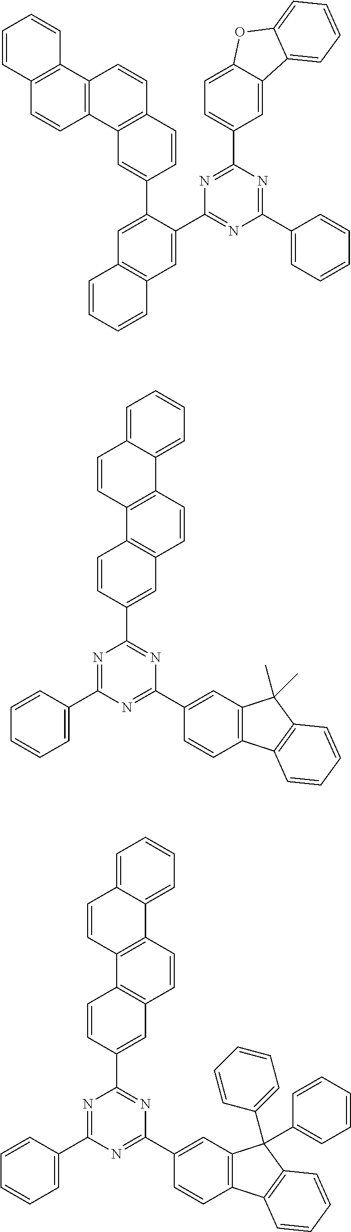 [SMILES: c1ccc(-c2nc(-c3ccc4oc5ccccc5c4c3)nc(-c3cc4ccccc4cc3-c3ccc4ccc5c6ccccc6ccc5c4c3)n2)cc1 ; CC1(C)c2ccccc2-c2ccc(-c3nc(-c4ccccc4)nc(-c4ccc5c(ccc6c7ccccc7ccc56)c4)n3)cc21 ; c1ccc(-c2nc(-c3ccc4c(c3)C(c3ccccc3)(c3ccccc3)c3ccccc3-4)nc(-c3ccc4c(ccc5c6ccccc6ccc45)c3)n2)cc1]

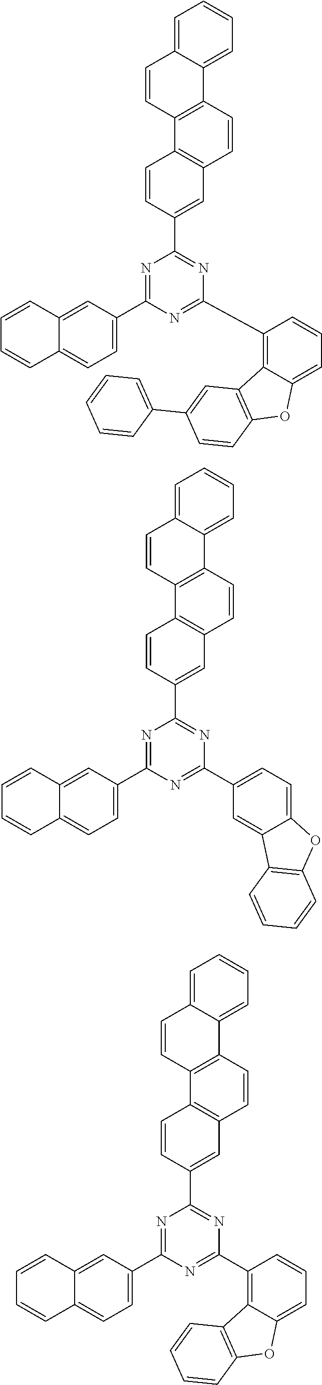

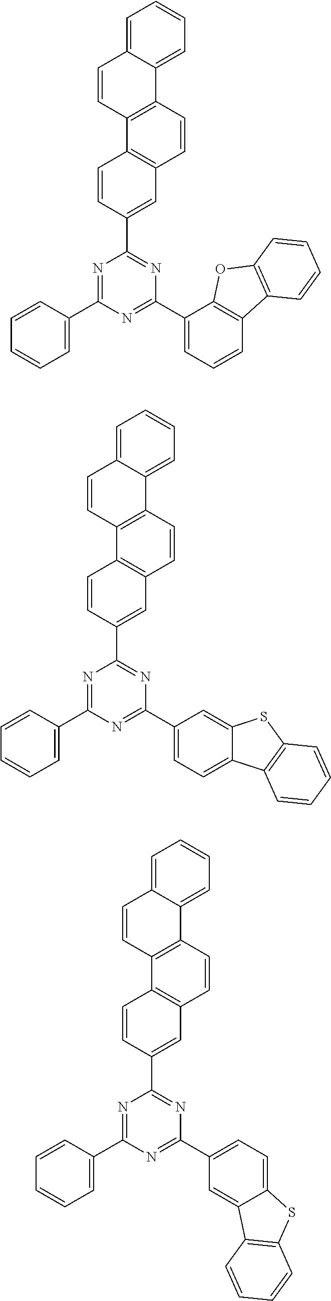 [SMILES: c1ccc(-c2nc(-c3ccc4c(ccc5c6ccccc6ccc45)c3)nc(-c3cccc4c3oc3ccccc34)n2)cc1 ; c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3ccc4sc5ccccc5c4c3)n2)cc1]

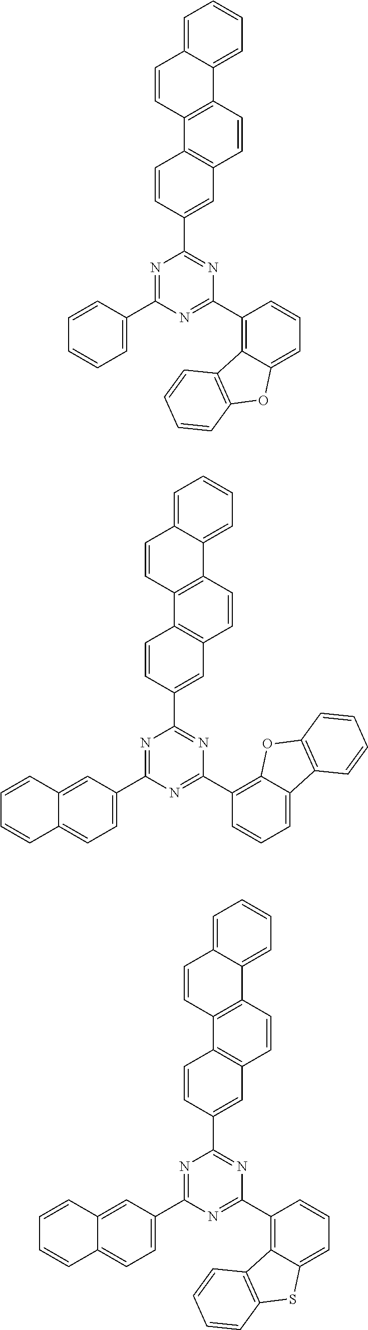

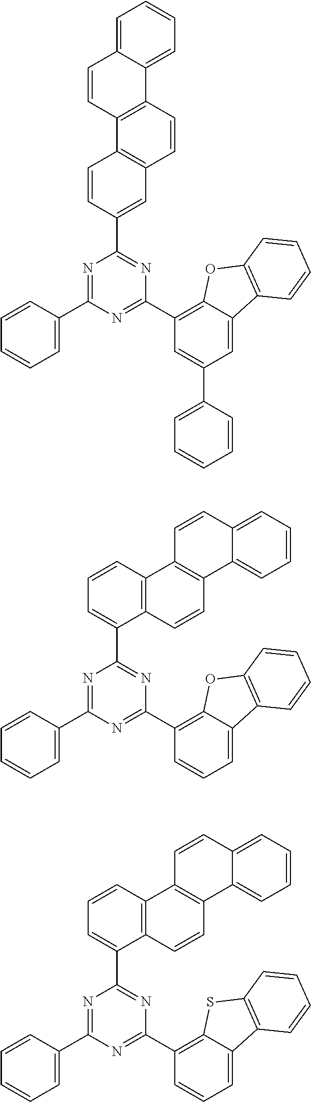

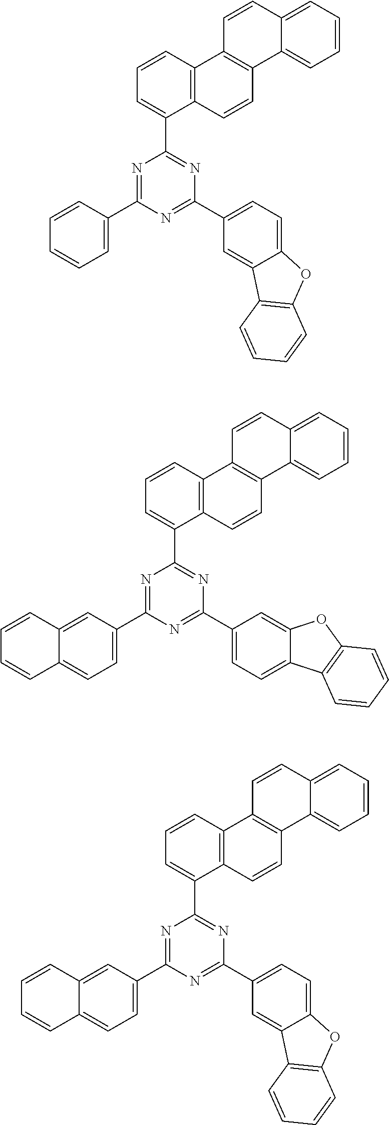 [SMILES: c1ccc(-c2nc(-c3ccc4oc5ccccc5c4c3)nc(-c3cccc4c3ccc3c5ccccc5ccc43)n2)cc1 ; c1ccc2cc(-c3nc(-c4ccc5c(c4)oc4ccccc45)nc(-c4cccc5c4ccc4c6ccccc6ccc54)n3)ccc2c1 ; c1ccc2cc(-c3nc(-c4ccc5oc6ccccc6c5c4)nc(-c4cccc5c4ccc4c6ccccc6ccc54)n3)ccc2c1]

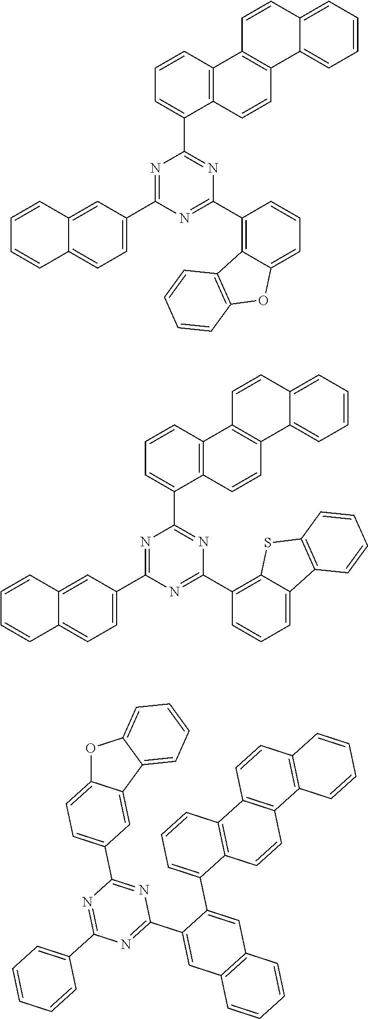 [SMILES: c1ccc2cc(-c3nc(-c4cccc5c4ccc4c6ccccc6ccc54)nc(-c4cccc5oc6ccccc6c45)n3)ccc2c1 ; c1ccc2cc(-c3nc(-c4cccc5c4ccc4c6ccccc6ccc54)nc(-c4cccc5c4sc4ccccc45)n3)ccc2c1 ; c1ccc(-c2nc(-c3ccc4oc5ccccc5c4c3)nc(-c3cc4ccccc4cc3-c3cccc4c3ccc3c5ccccc5ccc43)n2)cc1]

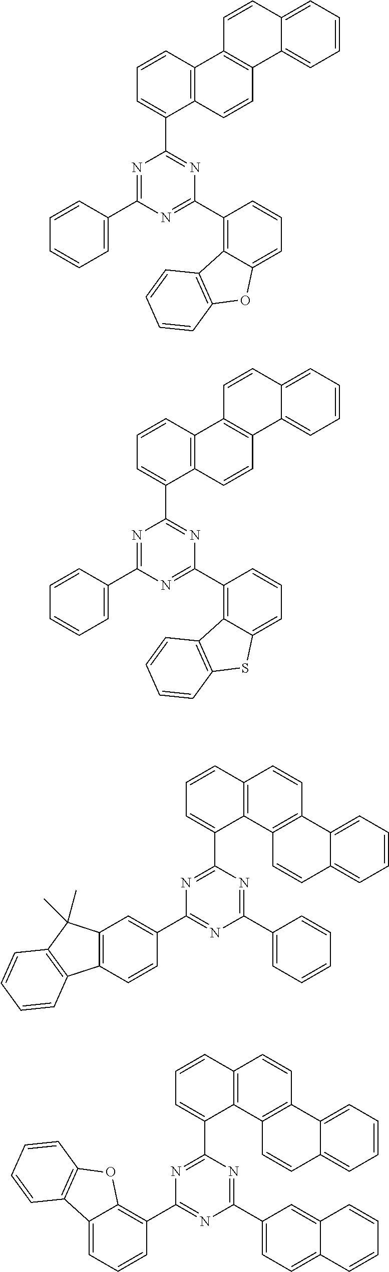 [SMILES: c1ccc(-c2nc(-c3cccc4c3ccc3c5ccccc5ccc43)nc(-c3cccc4oc5ccccc5c34)n2)cc1 ; c1ccc(-c2nc(-c3cccc4c3ccc3c5ccccc5ccc43)nc(-c3cccc4sc5ccccc5c34)n2)cc1 ; CC1(C)c2ccccc2-c2ccc(-c3nc(-c4ccccc4)nc(-c4cccc5ccc6c7ccccc7ccc6c45)n3)cc21 ; c1ccc2cc(-c3nc(-c4cccc5c4oc4ccccc45)nc(-c4cccc5ccc6c7ccccc7ccc6c45)n3)ccc2c1]

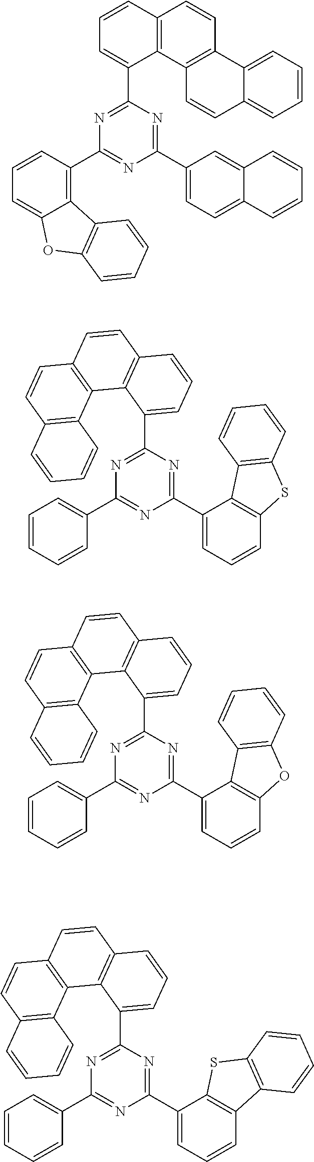 [SMILES: c1ccc2cc(-c3nc(-c4cccc5ccc6c7ccccc7ccc6c45)nc(-c4cccc5oc6ccccc6c45)n3)ccc2c1 ; c1ccc(-c2nc(-c3cccc4sc5ccccc5c34)nc(-c3cccc4ccc5ccc6ccccc6c5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3cccc4c3sc3ccccc34)nc(-c3cccc4ccc5ccc6ccccc6c5c34)n2)cc1]

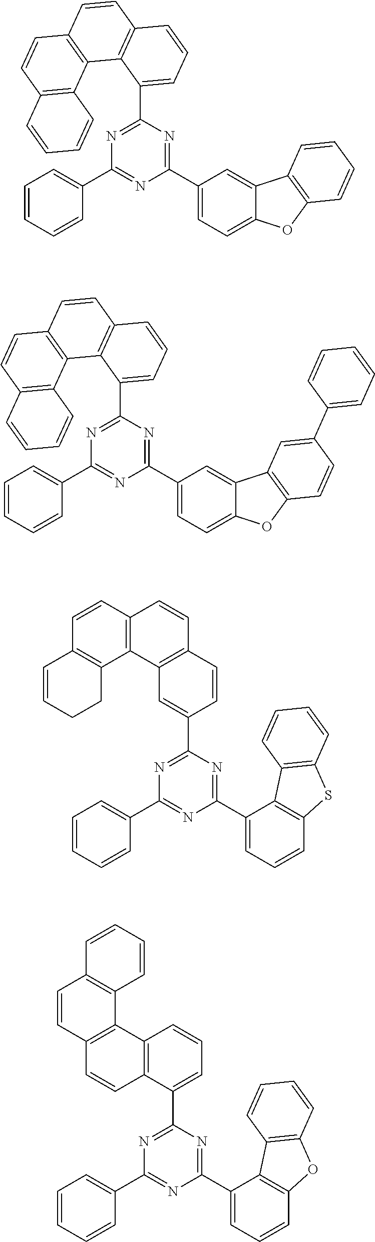 [SMILES: c1ccc(-c2nc(-c3ccc4oc5ccccc5c4c3)nc(-c3cccc4ccc5ccc6ccccc6c5c34)n2)cc1 ; c1ccc(-c2ccc3oc4ccc(-c5nc(-c6ccccc6)nc(-c6cccc7ccc8ccc9ccccc9c8c67)n5)cc4c3c2)cc1 ; C1=Cc2ccc3ccc4ccc(-c5nc(-c6ccccc6)nc(-c6cccc7sc8ccccc8c67)n5)cc4c3c2CC1 ; c1ccc(-c2nc(-c3cccc4c3ccc3ccc5ccccc5c34)nc(-c3cccc4oc5ccccc5c34)n2)cc1]

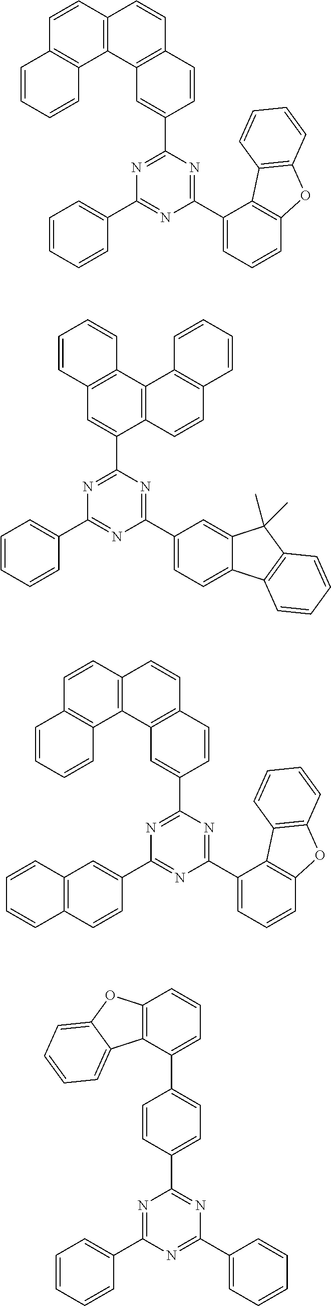 [SMILES: c1ccc(-c2nc(-c3ccc4ccc5ccc6ccccc6c5c4c3)nc(-c3cccc4oc5ccccc5c34)n2)cc1 ; CC1(C)c2ccccc2-c2ccc(-c3nc(-c4ccccc4)nc(-c4cc5ccccc5c5c4ccc4ccccc45)n3)cc21 ; c1ccc2cc(-c3nc(-c4ccc5ccc6ccc7ccccc7c6c5c4)nc(-c4cccc5oc6ccccc6c45)n3)ccc2c1 ; c1ccc(-c2nc(-c3ccccc3)nc(-c3ccc(-c4cccc5oc6ccccc6c45)cc3)n2)cc1]

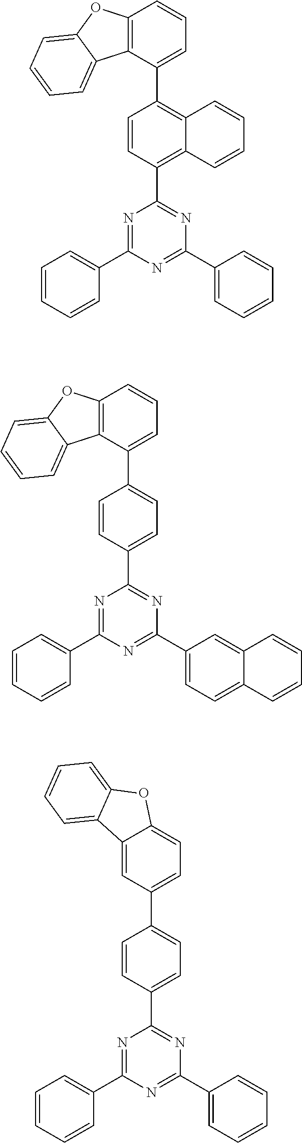 [SMILES: c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1 ; c1ccc(-c2nc(-c3ccccc3)nc(-c3ccc(-c4ccc5oc6ccccc6c5c4)cc3)n2)cc1]

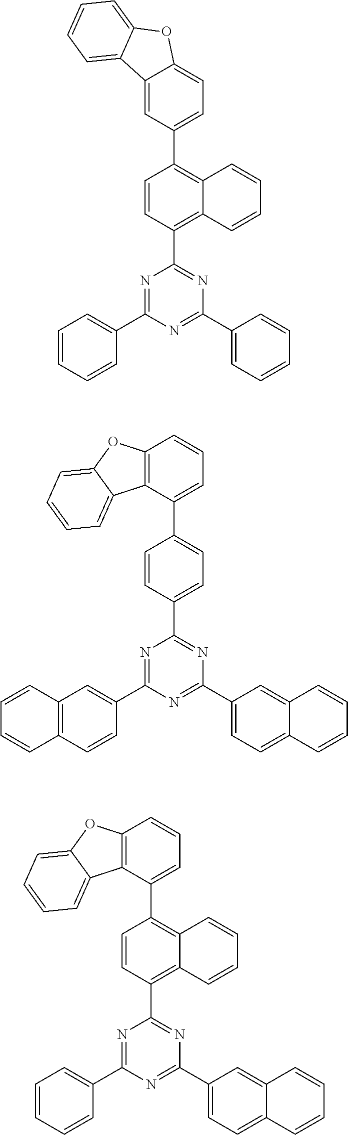 [SMILES: c1ccc(-c2nc(-c3ccccc3)nc(-c3ccc(-c4ccc5oc6ccccc6c5c4)c4ccccc34)n2)cc1 ; c1ccc2cc(-c3nc(-c4ccc(-c5cccc6oc7ccccc7c56)cc4)nc(-c4ccc5ccccc5c4)n3)ccc2c1 ; c1ccc(-c2nc(-c3ccc4ccccc4c3)nc(-c3ccc(-c4cccc5oc6ccccc6c45)c4ccccc34)n2)cc1]

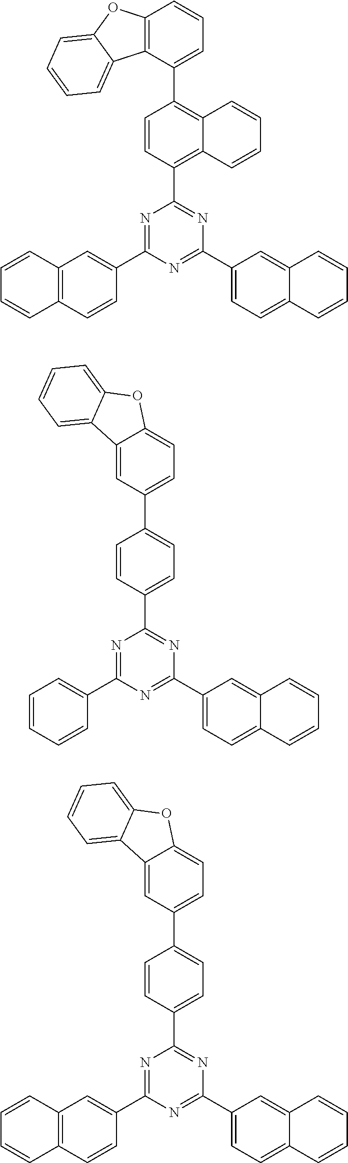 [SMILES: c1ccc2cc(-c3nc(-c4ccc5ccccc5c4)nc(-c4ccc(-c5cccc6oc7ccccc7c56)c5ccccc45)n3)ccc2c1 ; c1ccc(-c2nc(-c3ccc(-c4ccc5oc6ccccc6c5c4)cc3)nc(-c3ccc4ccccc4c3)n2)cc1 ; c1ccc2cc(-c3nc(-c4ccc(-c5ccc6oc7ccccc7c6c5)cc4)nc(-c4ccc5ccccc5c4)n3)ccc2c1]

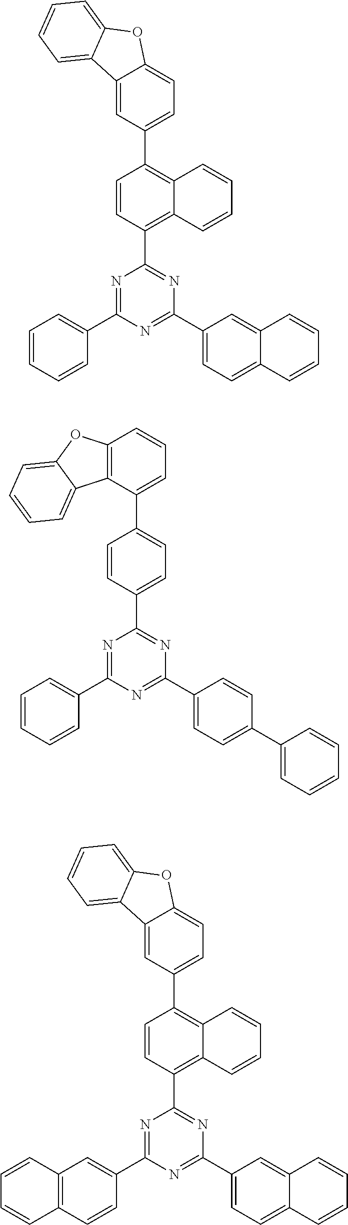 [SMILES: c1ccc(-c2nc(-c3ccc4ccccc4c3)nc(-c3ccc(-c4ccc5oc6ccccc6c5c4)c4ccccc34)n2)cc1 ; c1ccc(-c2ccc(-c3nc(-c4ccccc4)nc(-c4ccc(-c5cccc6oc7ccccc7c56)cc4)n3)cc2)cc1 ; c1ccc2cc(-c3nc(-c4ccc5ccccc5c4)nc(-c4ccc(-c5ccc6oc7ccccc7c6c5)c5ccccc45)n3)ccc2c1]

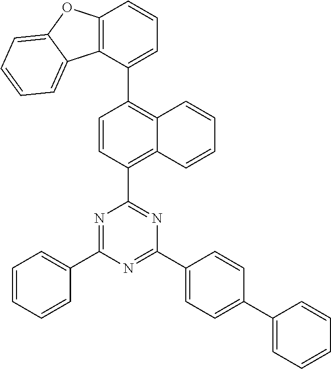 [SMILES: c1ccc(-c2ccc(-c3nc(-c4ccccc4)nc(-c4ccc(-c5cccc6oc7ccccc7c56)c5ccccc45)n3)cc2)cc1]

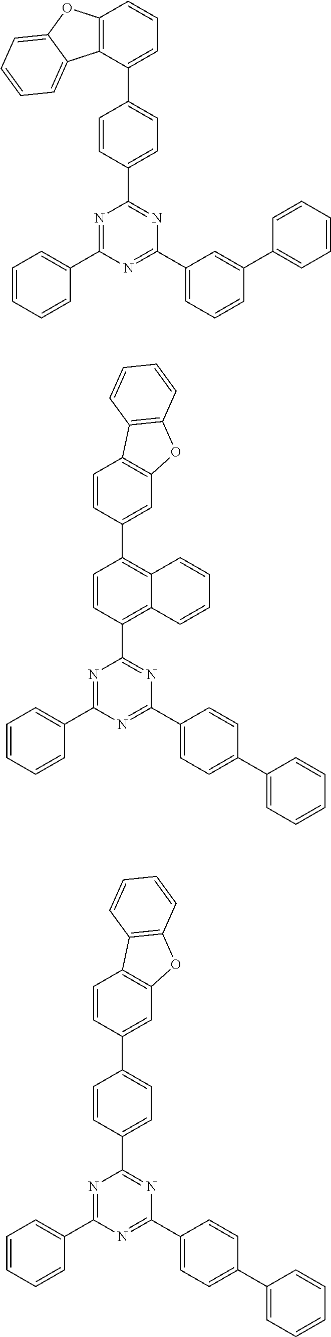 [SMILES: c1ccc(-c2cccc(-c3nc(-c4ccccc4)nc(-c4ccc(-c5cccc6oc7ccccc7c56)cc4)n3)c2)cc1 ; c1ccc(-c2ccc(-c3nc(-c4ccccc4)nc(-c4ccc(-c5ccc6c(c5)oc5ccccc56)c5ccccc45)n3)cc2)cc1 ; c1ccc(-c2ccc(-c3nc(-c4ccccc4)nc(-c4ccc(-c5ccc6c(c5)oc5ccccc56)cc4)n3)cc2)cc1]

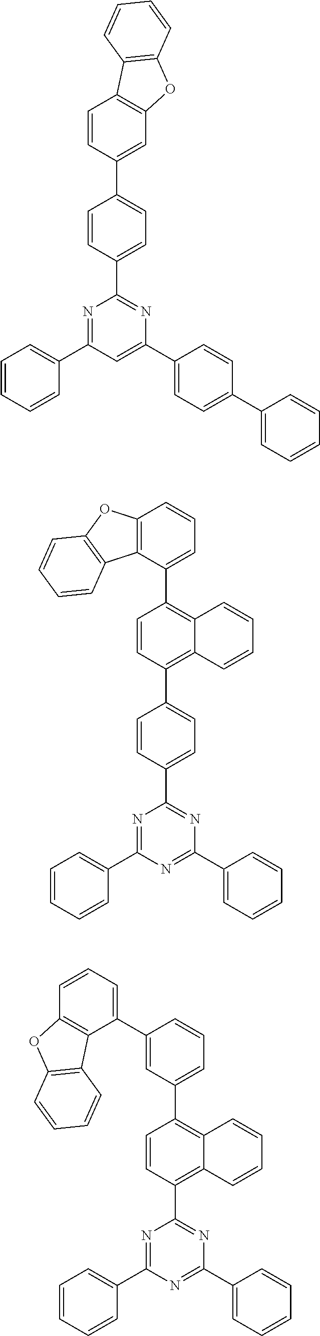 [SMILES: c1ccc(-c2ccc(-c3cc(-c4ccccc4)nc(-c4ccc(-c5ccc6c(c5)oc5ccccc56)cc4)n3)cc2)cc1 ; c1ccc(-c2nc(-c3ccccc3)nc(-c3ccc(-c4ccc(-c5cccc6oc7ccccc7c56)c5ccccc45)cc3)n2)cc1 ; c1ccc(-c2nc(-c3ccccc3)nc(-c3ccc(-c4cccc(-c5cccc6oc7ccccc7c56)c4)c4ccccc34)n2)cc1]

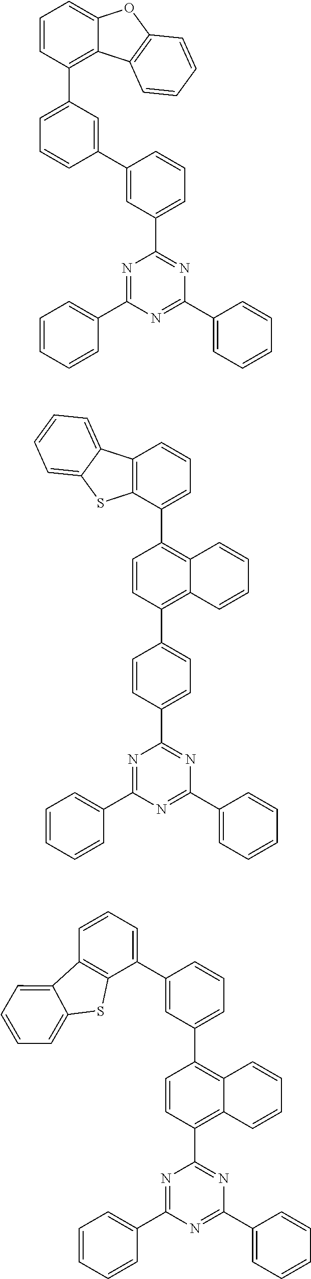 [SMILES: c1ccc(-c2nc(-c3ccccc3)nc(-c3cccc(-c4cccc(-c5cccc6oc7ccccc7c56)c4)c3)n2)cc1 ; c1ccc(-c2nc(-c3ccccc3)nc(-c3ccc(-c4ccc(-c5cccc6c5sc5ccccc56)c5ccccc45)cc3)n2)cc1 ; c1ccc(-c2nc(-c3ccccc3)nc(-c3ccc(-c4cccc(-c5cccc6c5sc5ccccc56)c4)c4ccccc34)n2)cc1]

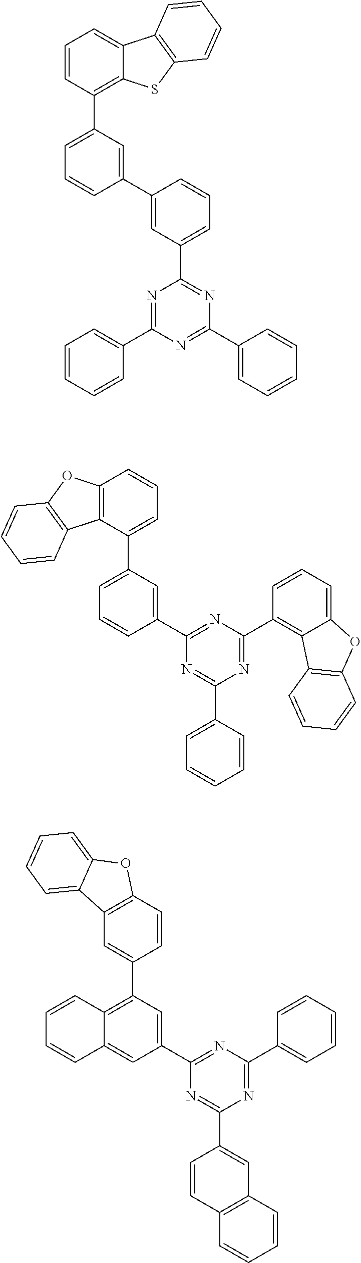 [SMILES: c1ccc(-c2nc(-c3ccccc3)nc(-c3cccc(-c4cccc(-c5cccc6c5sc5ccccc56)c4)c3)n2)cc1 ; c1ccc(-c2nc(-c3cccc(-c4cccc5oc6ccccc6c45)c3)nc(-c3cccc4oc5ccccc5c34)n2)cc1 ; c1ccc(-c2nc(-c3ccc4ccccc4c3)nc(-c3cc(-c4ccc5oc6ccccc6c5c4)c4ccccc4c3)n2)cc1]

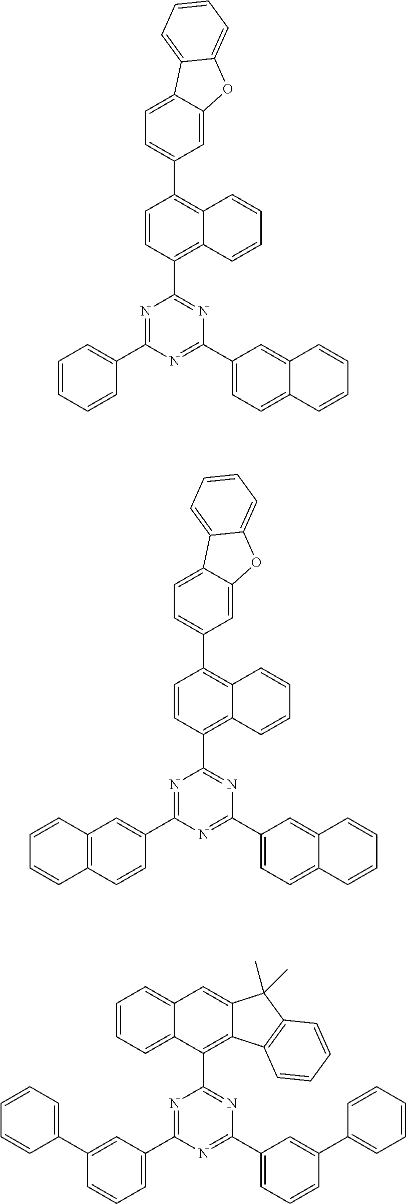 [SMILES: c1ccc(-c2nc(-c3ccc4ccccc4c3)nc(-c3ccc(-c4ccc5c(c4)oc4ccccc45)c4ccccc34)n2)cc1 ; c1ccc2cc(-c3nc(-c4ccc5ccccc5c4)nc(-c4ccc(-c5ccc6c(c5)oc5ccccc56)c5ccccc45)n3)ccc2c1 ; CC1(C)c2ccccc2-c2c1cc1ccccc1c2-c1nc(-c2cccc(-c3ccccc3)c2)nc(-c2cccc(-c3ccccc3)c2)n1]

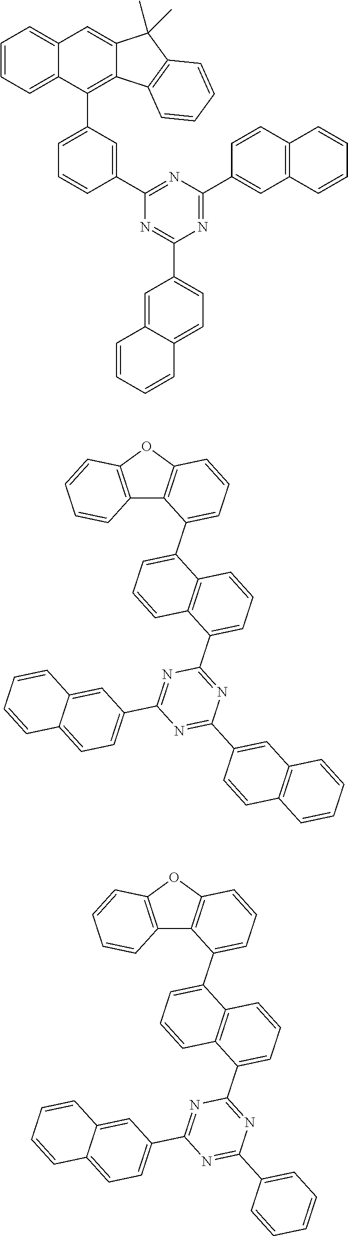 [SMILES: CC1(C)c2ccccc2-c2c1cc1ccccc1c2-c1cccc(-c2nc(-c3ccc4ccccc4c3)nc(-c3ccc4ccccc4c3)n2)c1 ; c1ccc2cc(-c3nc(-c4ccc5ccccc5c4)nc(-c4cccc5c(-c6cccc7oc8ccccc8c67)cccc45)n3)ccc2c1 ; c1ccc(-c2nc(-c3ccc4ccccc4c3)nc(-c3cccc4c(-c5cccc6oc7ccccc7c56)cccc34)n2)cc1]

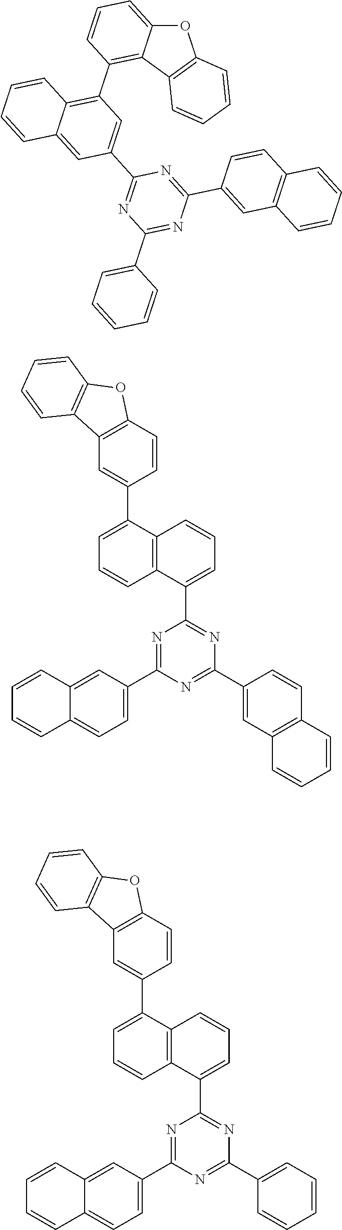 [SMILES: c1ccc(-c2nc(-c3ccc4ccccc4c3)nc(-c3cc(-c4cccc5oc6ccccc6c45)c4ccccc4c3)n2)cc1 ; c1ccc2cc(-c3nc(-c4ccc5ccccc5c4)nc(-c4cccc5c(-c6ccc7oc8ccccc8c7c6)cccc45)n3)ccc2c1 ; c1ccc(-c2nc(-c3ccc4ccccc4c3)nc(-c3cccc4c(-c5ccc6oc7ccccc7c6c5)cccc34)n2)cc1]

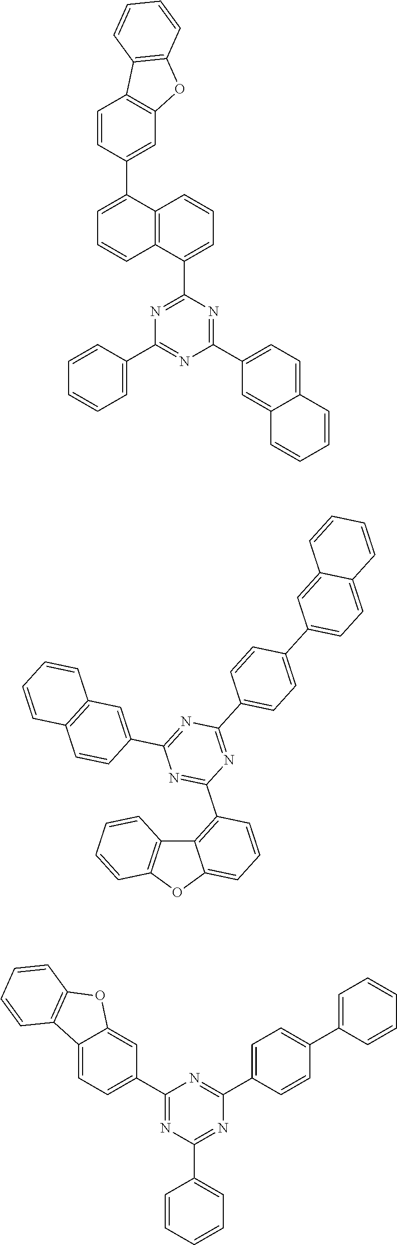 [SMILES: c1ccc(-c2nc(-c3ccc4ccccc4c3)nc(-c3cccc4c(-c5ccc6c(c5)oc5ccccc56)cccc34)n2)cc1 ; c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3nc(-c4ccccc4)nc(-c4ccc5c(c4)oc4ccccc45)n3)cc2)cc1]

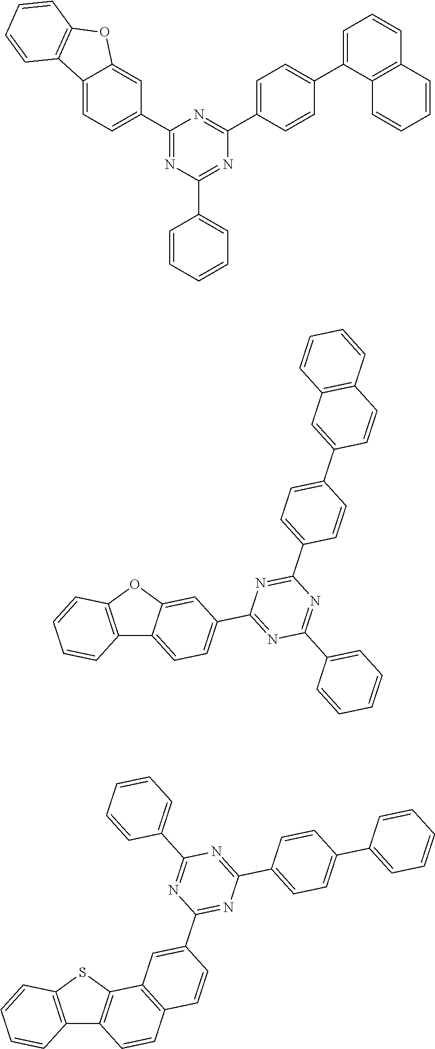 [SMILES: c1ccc(-c2nc(-c3ccc(-c4cccc5ccccc45)cc3)nc(-c3ccc4c(c3)oc3ccccc34)n2)cc1 ; c1ccc(-c2nc(-c3ccc(-c4ccc5ccccc5c4)cc3)nc(-c3ccc4c(c3)oc3ccccc34)n2)cc1 ; c1ccc(-c2ccc(-c3nc(-c4ccccc4)nc(-c4ccc5ccc6c7ccccc7sc6c5c4)n3)cc2)cc1]

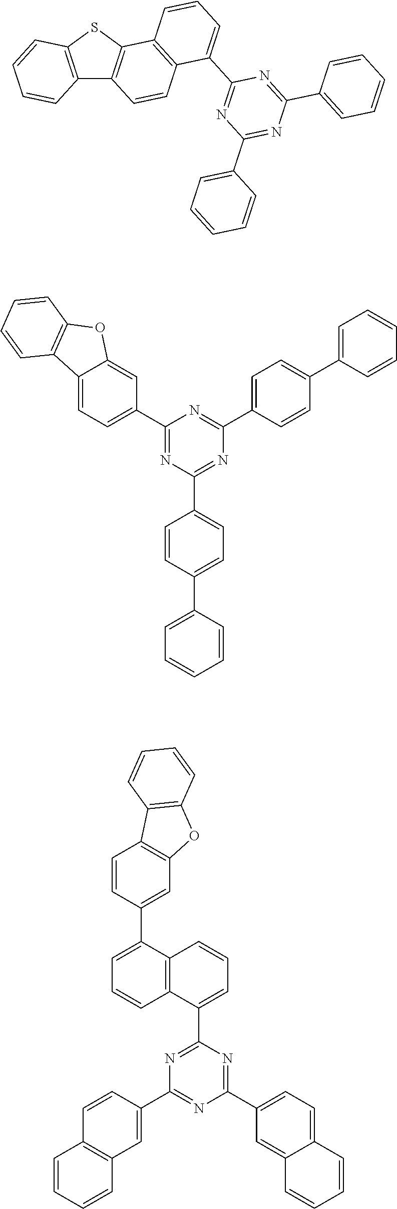 [SMILES: c1ccc(-c2nc(-c3ccccc3)nc(-c3cccc4c3ccc3c5ccccc5sc43)n2)cc1 ; c1ccc(-c2ccc(-c3nc(-c4ccc(-c5ccccc5)cc4)nc(-c4ccc5c(c4)oc4ccccc45)n3)cc2)cc1 ; c1ccc2cc(-c3nc(-c4ccc5ccccc5c4)nc(-c4cccc5c(-c6ccc7c(c6)oc6ccccc67)cccc45)n3)ccc2c1]

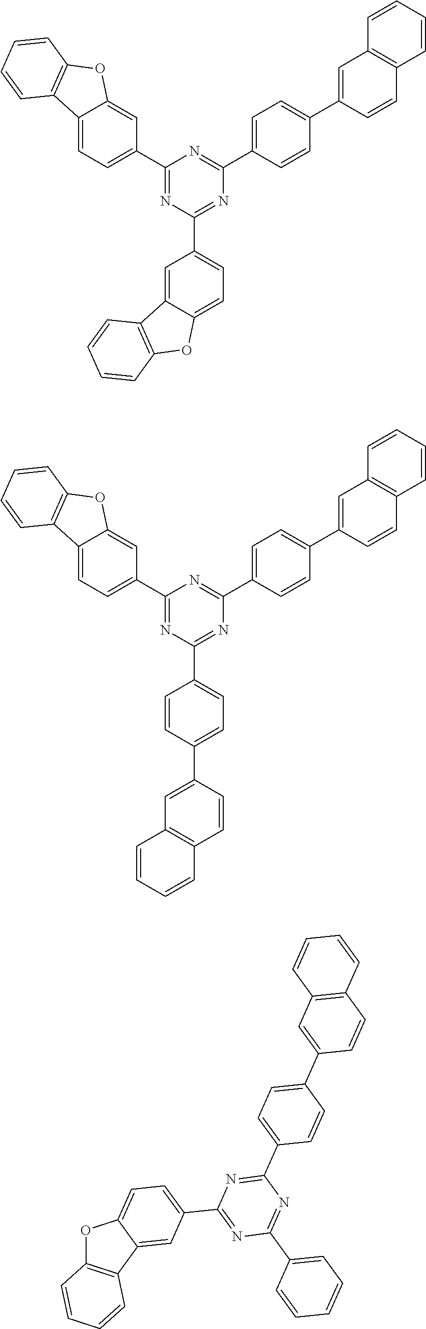 [SMILES: c1ccc2cc(-c3ccc(-c4nc(-c5ccc6c(c5)oc5ccccc56)nc(-c5ccc6oc7ccccc7c6c5)n4)cc3)ccc2c1 ; c1ccc2cc(-c3ccc(-c4nc(-c5ccc(-c6ccc7ccccc7c6)cc5)nc(-c5ccc6c(c5)oc5ccccc56)n4)cc3)ccc2c1 ; c1ccc(-c2nc(-c3ccc(-c4ccc5ccccc5c4)cc3)nc(-c3ccc4oc5ccccc5c4c3)n2)cc1]

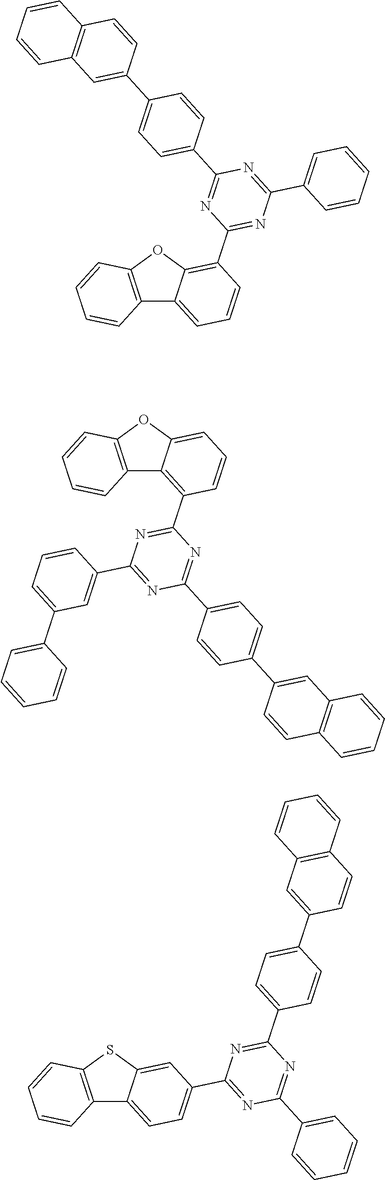

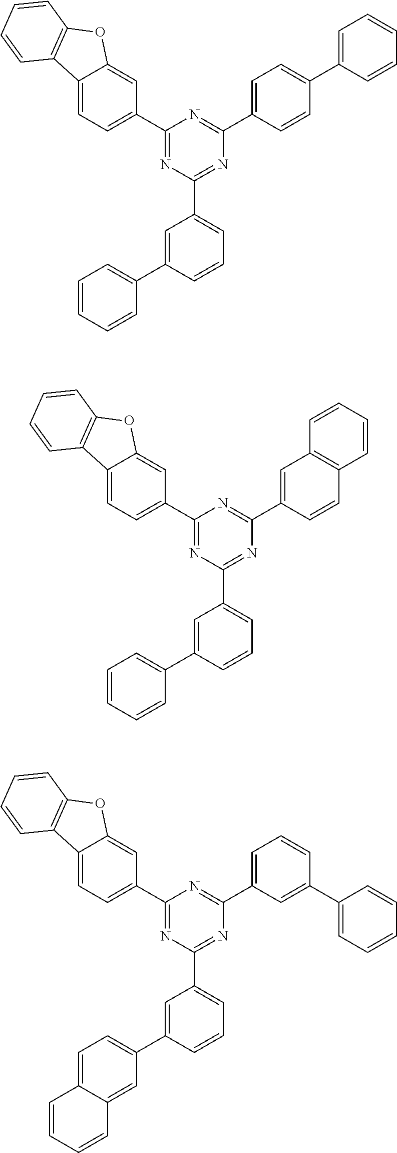 [SMILES: c1ccc(-c2ccc(-c3nc(-c4cccc(-c5ccccc5)c4)nc(-c4ccc5c(c4)oc4ccccc45)n3)cc2)cc1 ; c1ccc(-c2cccc(-c3nc(-c4ccc5ccccc5c4)nc(-c4ccc5c(c4)oc4ccccc45)n3)c2)cc1 ; c1ccc(-c2cccc(-c3nc(-c4cccc(-c5ccc6ccccc6c5)c4)nc(-c4ccc5c(c4)oc4ccccc45)n3)c2)cc1]

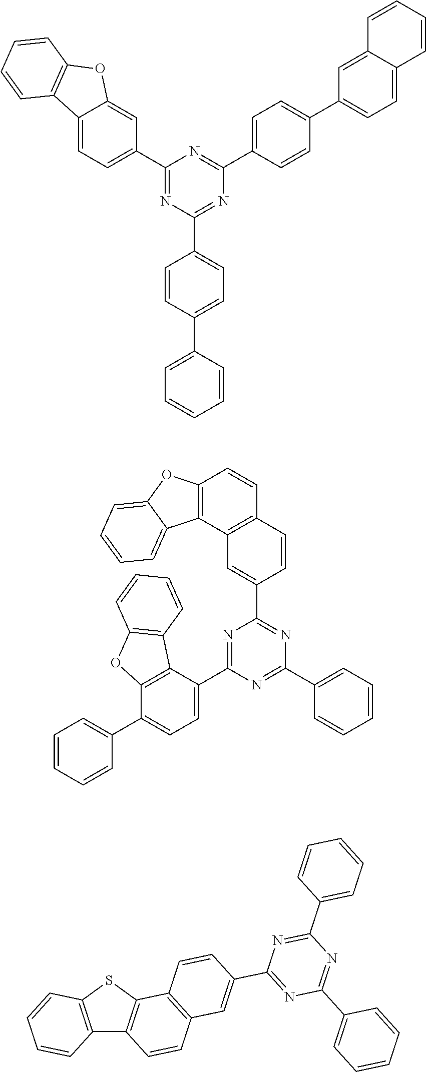 [SMILES: c1ccc(-c2ccc(-c3nc(-c4ccc(-c5ccc6ccccc6c5)cc4)nc(-c4ccc5c(c4)oc4ccccc45)n3)cc2)cc1 ; c1ccc(-c2nc(-c3ccc4ccc5oc6ccccc6c5c4c3)nc(-c3ccc(-c4ccccc4)c4oc5ccccc5c34)n2)cc1 ; c1ccc(-c2nc(-c3ccccc3)nc(-c3ccc4c(ccc5c6ccccc6sc45)c3)n2)cc1]

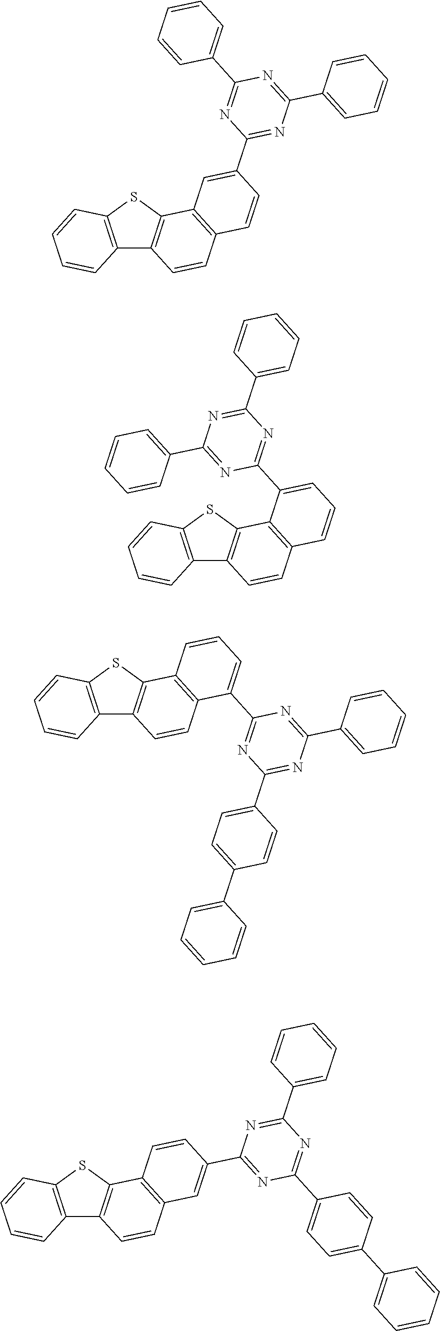 [SMILES: c1ccc(-c2nc(-c3ccccc3)nc(-c3ccc4ccc5c6ccccc6sc5c4c3)n2)cc1 ; c1ccc(-c2nc(-c3ccccc3)nc(-c3cccc4ccc5c6ccccc6sc5c34)n2)cc1 ; c1ccc(-c2ccc(-c3nc(-c4ccccc4)nc(-c4cccc5c4ccc4c6ccccc6sc54)n3)cc2)cc1 ; c1ccc(-c2ccc(-c3nc(-c4ccccc4)nc(-c4ccc5c(ccc6c7ccccc7sc56)c4)n3)cc2)cc1]

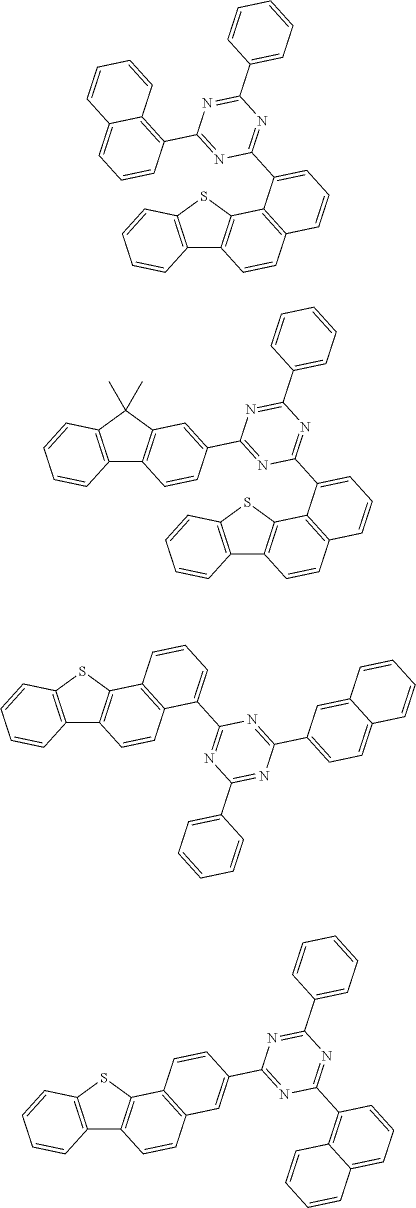 [SMILES: c1ccc(-c2nc(-c3cccc4ccccc34)nc(-c3cccc4ccc5c6ccccc6sc5c34)n2)cc1 ; CC1(C)c2ccccc2-c2ccc(-c3nc(-c4ccccc4)nc(-c4cccc5ccc6c7ccccc7sc6c45)n3)cc21 ; c1ccc(-c2nc(-c3ccc4ccccc4c3)nc(-c3cccc4c3ccc3c5ccccc5sc43)n2)cc1 ; c1ccc(-c2nc(-c3ccc4c(ccc5c6ccccc6sc45)c3)nc(-c3cccc4ccccc34)n2)cc1]

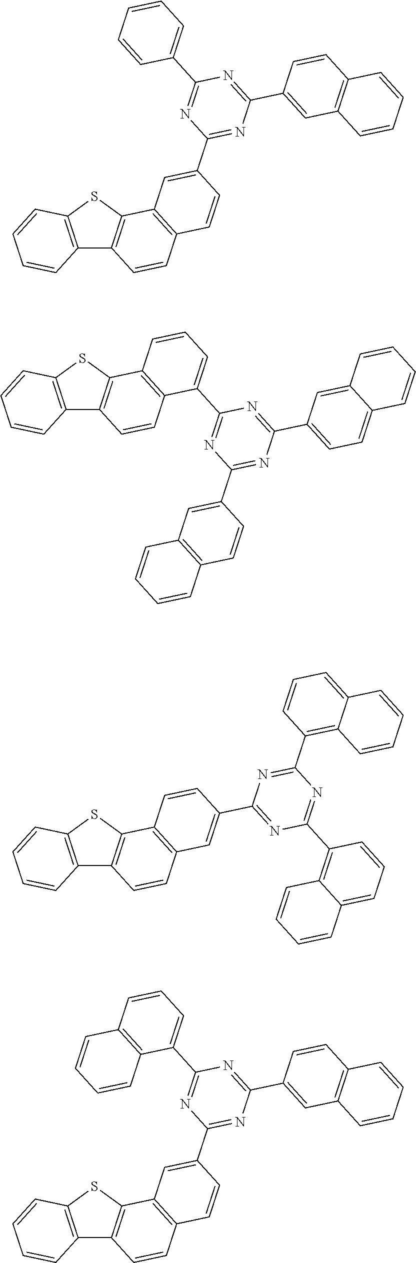 [SMILES: c1ccc(-c2nc(-c3ccc4ccccc4c3)nc(-c3ccc4ccc5c6ccccc6sc5c4c3)n2)cc1 ; c1ccc2cc(-c3nc(-c4ccc5ccccc5c4)nc(-c4cccc5c4ccc4c6ccccc6sc54)n3)ccc2c1 ; c1ccc2c(-c3nc(-c4ccc5c(ccc6c7ccccc7sc56)c4)nc(-c4cccc5ccccc45)n3)cccc2c1 ; c1ccc2cc(-c3nc(-c4ccc5ccc6c7ccccc7sc6c5c4)nc(-c4cccc5ccccc45)n3)ccc2c1]

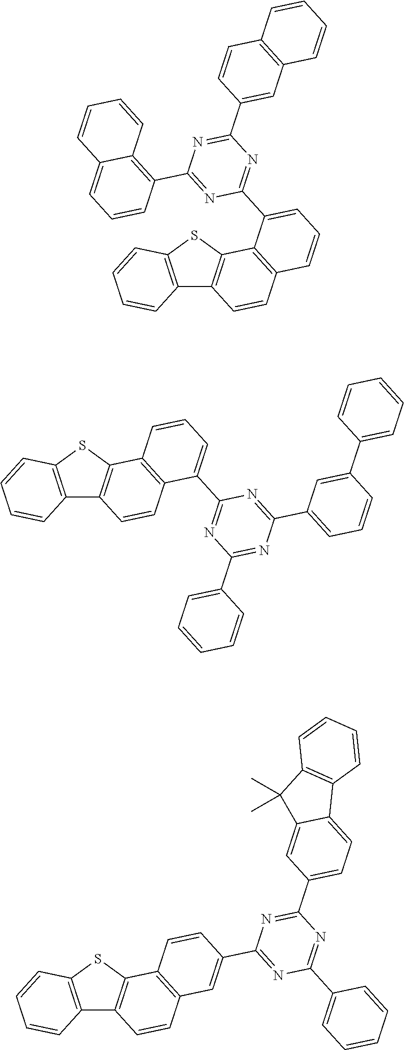 [SMILES: c1ccc2cc(-c3nc(-c4cccc5ccccc45)nc(-c4cccc5ccc6c7ccccc7sc6c45)n3)ccc2c1 ; c1ccc(-c2cccc(-c3nc(-c4ccccc4)nc(-c4cccc5c4ccc4c6ccccc6sc54)n3)c2)cc1 ; CC1(C)c2ccccc2-c2ccc(-c3nc(-c4ccccc4)nc(-c4ccc5c(ccc6c7ccccc7sc56)c4)n3)cc21]

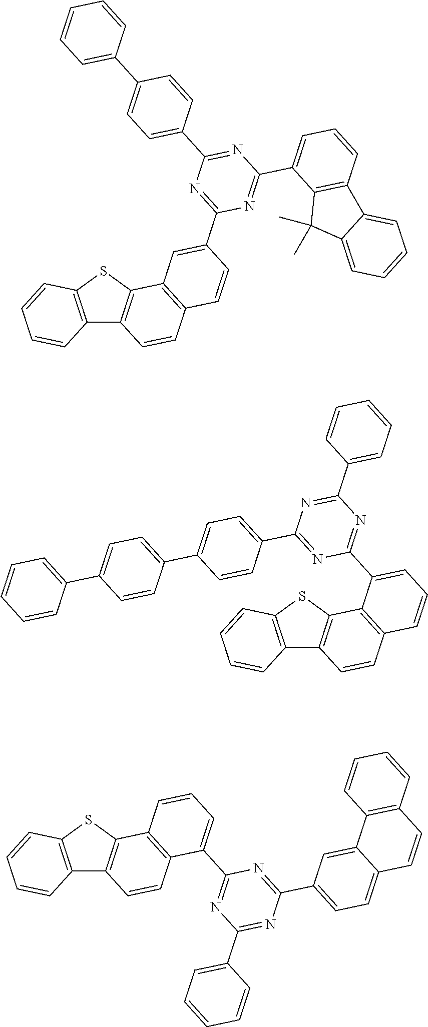 [SMILES: CC1(C)c2ccccc2-c2cccc(-c3nc(-c4ccc(-c5ccccc5)cc4)nc(-c4ccc5ccc6c7ccccc7sc6c5c4)n3)c21 ; c1ccc(-c2ccc(-c3ccc(-c4nc(-c5ccccc5)nc(-c5cccc6ccc7c8ccccc8sc7c56)n4)cc3)cc2)cc1 ; c1ccc(-c2nc(-c3ccc4ccc5ccccc5c4c3)nc(-c3cccc4c3ccc3c5ccccc5sc43)n2)cc1]

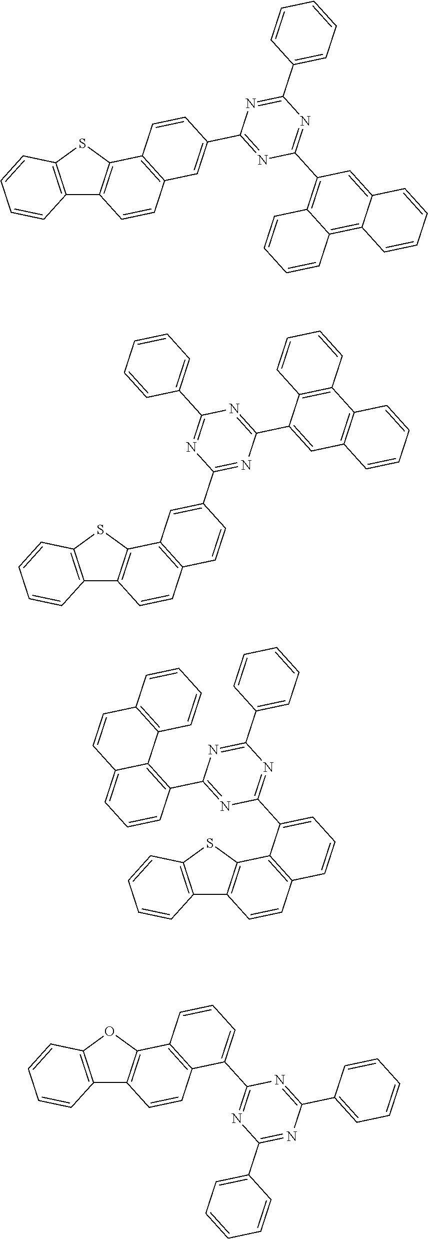 [SMILES: c1ccc(-c2nc(-c3ccc4c(ccc5c6ccccc6sc45)c3)nc(-c3cc4ccccc4c4ccccc34)n2)cc1 ; c1ccc(-c2nc(-c3ccc4ccc5c6ccccc6sc5c4c3)nc(-c3cc4ccccc4c4ccccc34)n2)cc1 ; c1ccc(-c2nc(-c3cccc4ccc5ccccc5c34)nc(-c3cccc4ccc5c6ccccc6sc5c34)n2)cc1 ; c1ccc(-c2nc(-c3ccccc3)nc(-c3cccc4c3ccc3c5ccccc5oc43)n2)cc1]

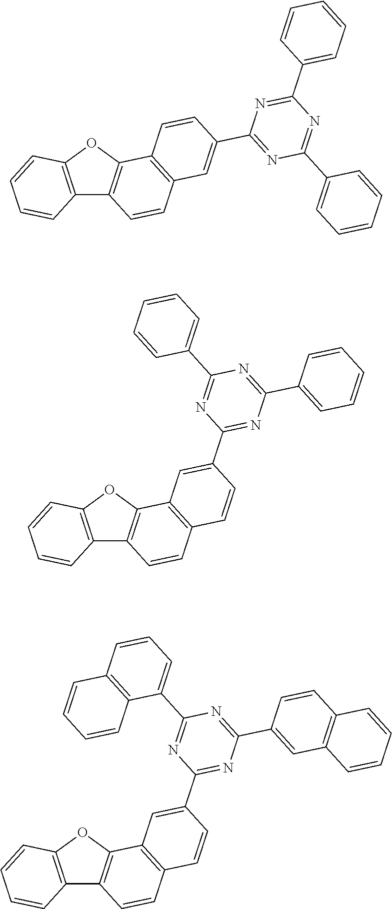 [SMILES: c1ccc(-c2nc(-c3ccccc3)nc(-c3ccc4c(ccc5c6ccccc6oc45)c3)n2)cc1 ; c1ccc(-c2nc(-c3ccccc3)nc(-c3ccc4ccc5c6ccccc6oc5c4c3)n2)cc1 ; c1ccc2cc(-c3nc(-c4ccc5ccc6c7ccccc7oc6c5c4)nc(-c4cccc5ccccc45)n3)ccc2c1]

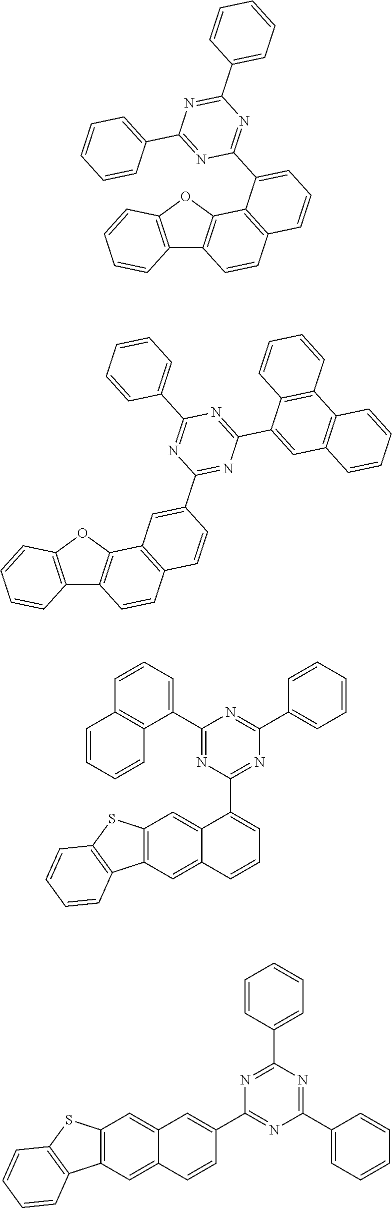 [SMILES: c1ccc(-c2nc(-c3ccccc3)nc(-c3cccc4ccc5c6ccccc6oc5c34)n2)cc1 ; c1ccc(-c2nc(-c3ccc4ccc5c6ccccc6oc5c4c3)nc(-c3cc4ccccc4c4ccccc34)n2)cc1 ; c1ccc(-c2nc(-c3cccc4ccccc34)nc(-c3cccc4cc5c(cc34)sc3ccccc35)n2)cc1 ; c1ccc(-c2nc(-c3ccccc3)nc(-c3ccc4cc5c(cc4c3)sc3ccccc35)n2)cc1]

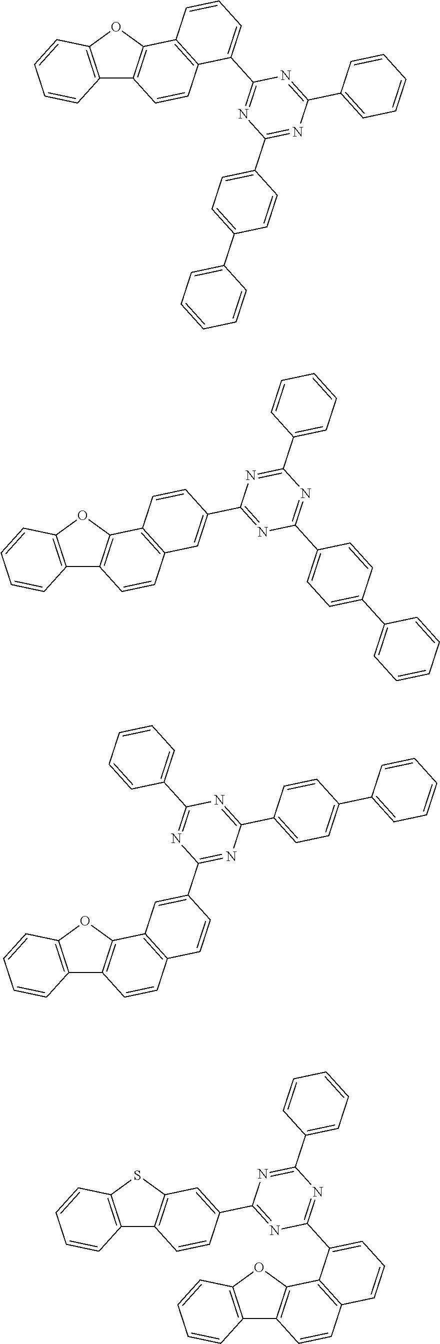 [SMILES: c1ccc(-c2ccc(-c3nc(-c4ccccc4)nc(-c4cccc5c4ccc4c6ccccc6oc54)n3)cc2)cc1 ; c1ccc(-c2ccc(-c3nc(-c4ccccc4)nc(-c4ccc5c(ccc6c7ccccc7oc56)c4)n3)cc2)cc1 ; c1ccc(-c2ccc(-c3nc(-c4ccccc4)nc(-c4ccc5ccc6c7ccccc7oc6c5c4)n3)cc2)cc1 ; c1ccc(-c2nc(-c3ccc4c(c3)sc3ccccc34)nc(-c3cccc4ccc5c6ccccc6oc5c34)n2)cc1]

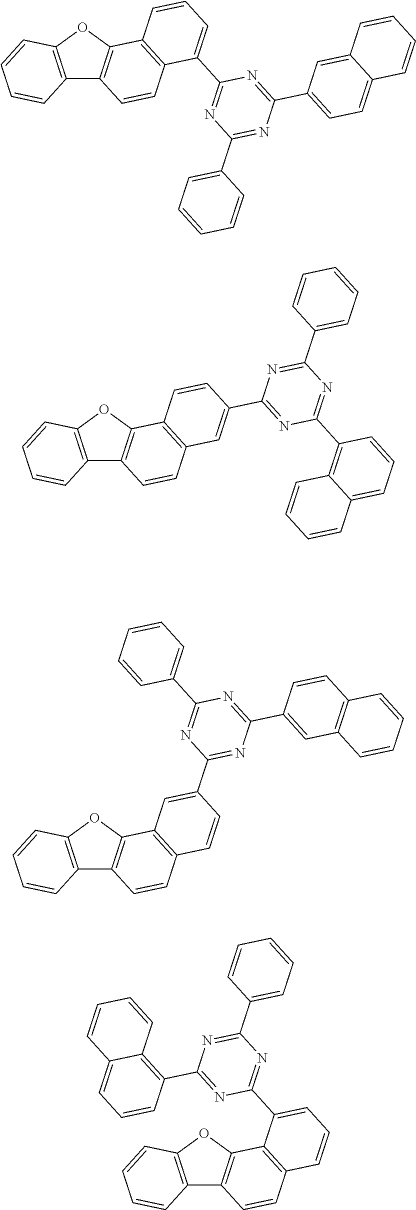 [SMILES: c1ccc(-c2nc(-c3ccc4ccccc4c3)nc(-c3cccc4c3ccc3c5ccccc5oc43)n2)cc1 ; c1ccc(-c2nc(-c3ccc4c(ccc5c6ccccc6oc45)c3)nc(-c3cccc4ccccc34)n2)cc1 ; c1ccc(-c2nc(-c3ccc4ccccc4c3)nc(-c3ccc4ccc5c6ccccc6oc5c4c3)n2)cc1 ; c1ccc(-c2nc(-c3cccc4ccccc34)nc(-c3cccc4ccc5c6ccccc6oc5c34)n2)cc1]

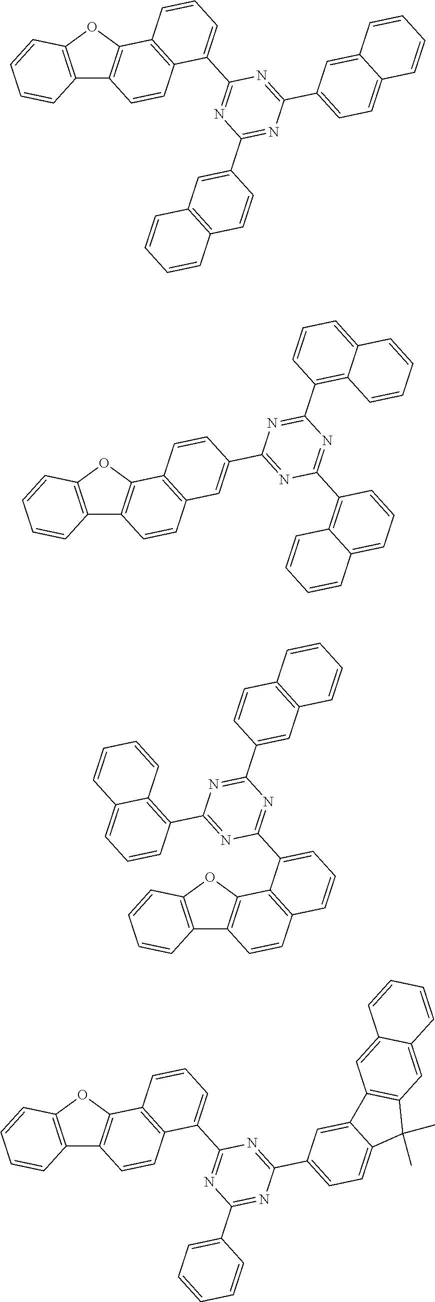 [SMILES: c1ccc2cc(-c3nc(-c4ccc5ccccc5c4)nc(-c4cccc5c4ccc4c6ccccc6oc54)n3)ccc2c1 ; c1ccc2c(-c3nc(-c4ccc5c(ccc6c7ccccc7oc56)c4)nc(-c4cccc5ccccc45)n3)cccc2c1 ; c1ccc2cc(-c3nc(-c4cccc5ccccc45)nc(-c4cccc5ccc6c7ccccc7oc6c45)n3)ccc2c1 ; CC1(C)c2ccc(-c3nc(-c4ccccc4)nc(-c4cccc5c4ccc4c6ccccc6oc54)n3)cc2-c2cc3ccccc3cc21]

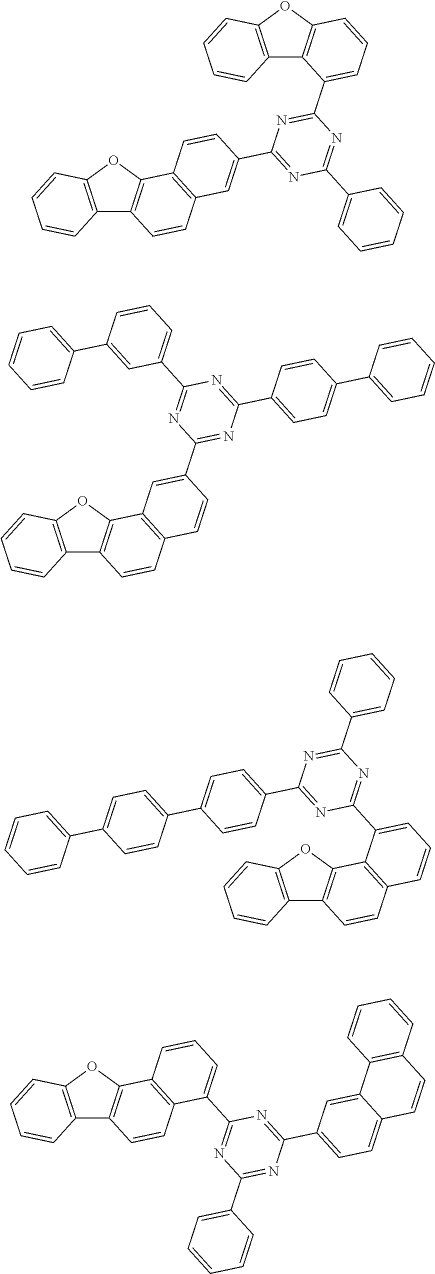 [SMILES: c1ccc(-c2nc(-c3ccc4c(ccc5c6ccccc6oc45)c3)nc(-c3cccc4oc5ccccc5c34)n2)cc1 ; c1ccc(-c2ccc(-c3nc(-c4cccc(-c5ccccc5)c4)nc(-c4ccc5ccc6c7ccccc7oc6c5c4)n3)cc2)cc1 ; c1ccc(-c2ccc(-c3ccc(-c4nc(-c5ccccc5)nc(-c5cccc6ccc7c8ccccc8oc7c56)n4)cc3)cc2)cc1 ; c1ccc(-c2nc(-c3ccc4ccc5ccccc5c4c3)nc(-c3cccc4c3ccc3c5ccccc5oc43)n2)cc1]

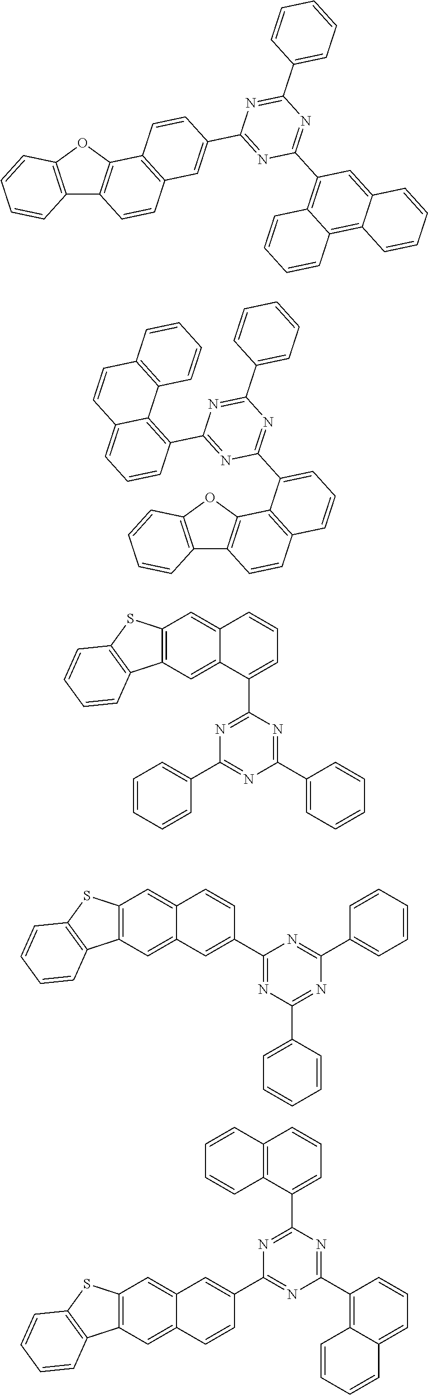

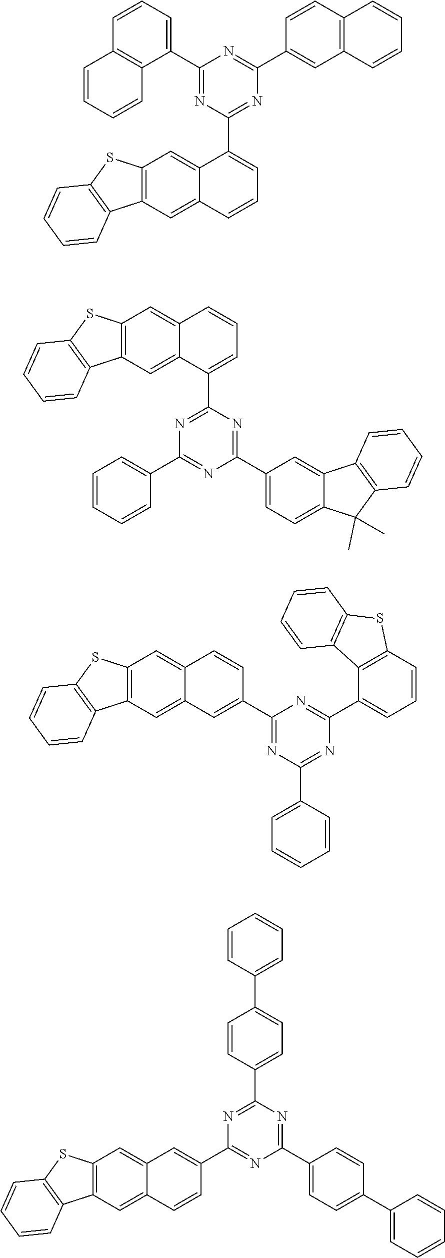

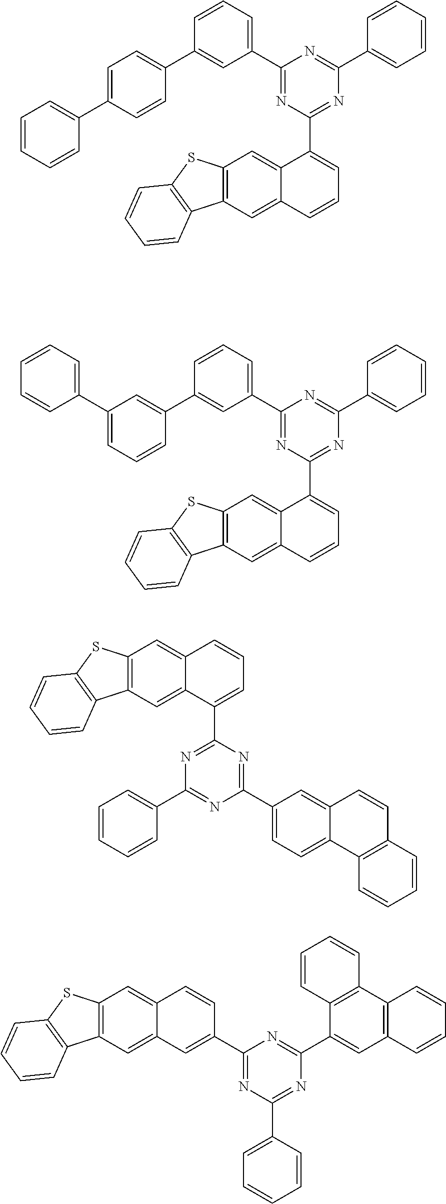 [SMILES: c1ccc(-c2ccc(-c3cccc(-c4nc(-c5ccccc5)nc(-c5cccc6cc7c(cc56)sc5ccccc57)n4)c3)cc2)cc1 ; c1ccc(-c2cccc(-c3cccc(-c4nc(-c5ccccc5)nc(-c5cccc6cc7c(cc56)sc5ccccc57)n4)c3)c2)cc1 ; c1ccc(-c2nc(-c3ccc4c(ccc5ccccc54)c3)nc(-c3cccc4cc5sc6ccccc6c5cc34)n2)cc1 ; c1ccc(-c2nc(-c3ccc4cc5sc6ccccc6c5cc4c3)nc(-c3cc4ccccc4c4ccccc34)n2)cc1]

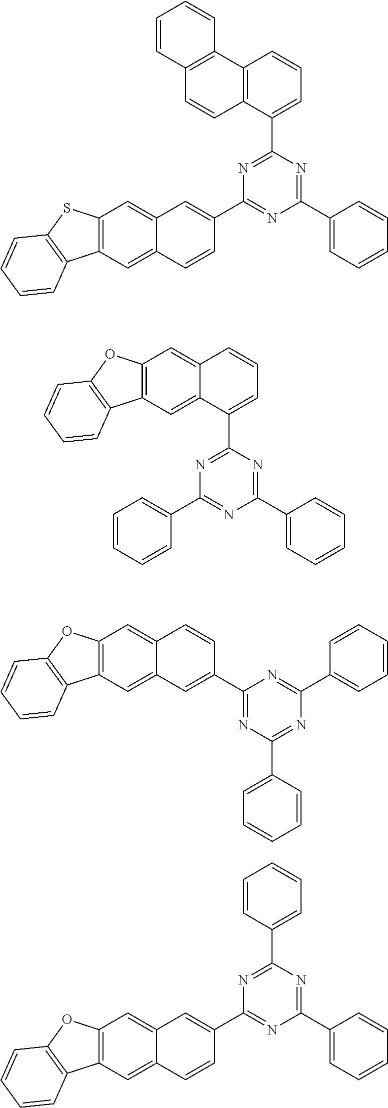

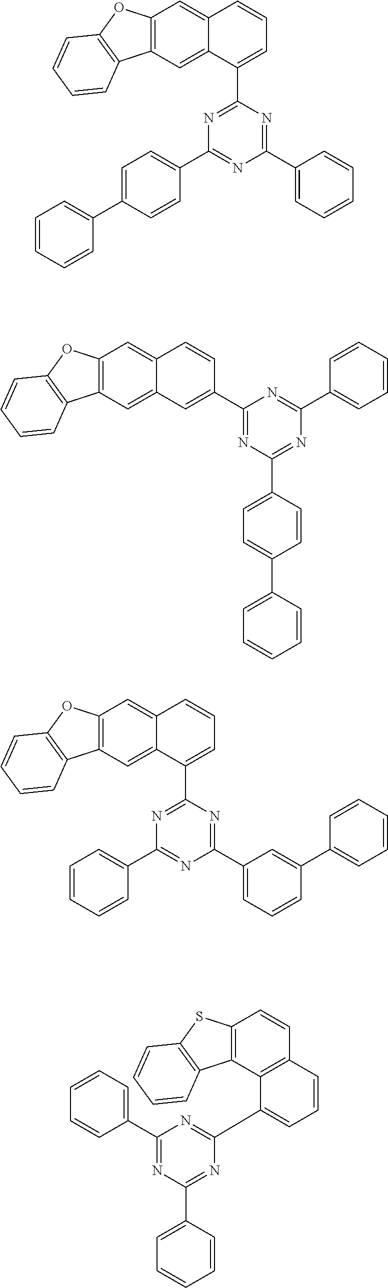 [SMILES: c1ccc(-c2ccc(-c3nc(-c4ccccc4)nc(-c4cccc5cc6oc7ccccc7c6cc45)n3)cc2)cc1 ; c1ccc(-c2ccc(-c3nc(-c4ccccc4)nc(-c4ccc5cc6oc7ccccc7c6cc5c4)n3)cc2)cc1 ; c1ccc(-c2cccc(-c3nc(-c4ccccc4)nc(-c4cccc5cc6oc7ccccc7c6cc45)n3)c2)cc1 ; c1ccc(-c2nc(-c3ccccc3)nc(-c3cccc4ccc5sc6ccccc6c5c34)n2)cc1]

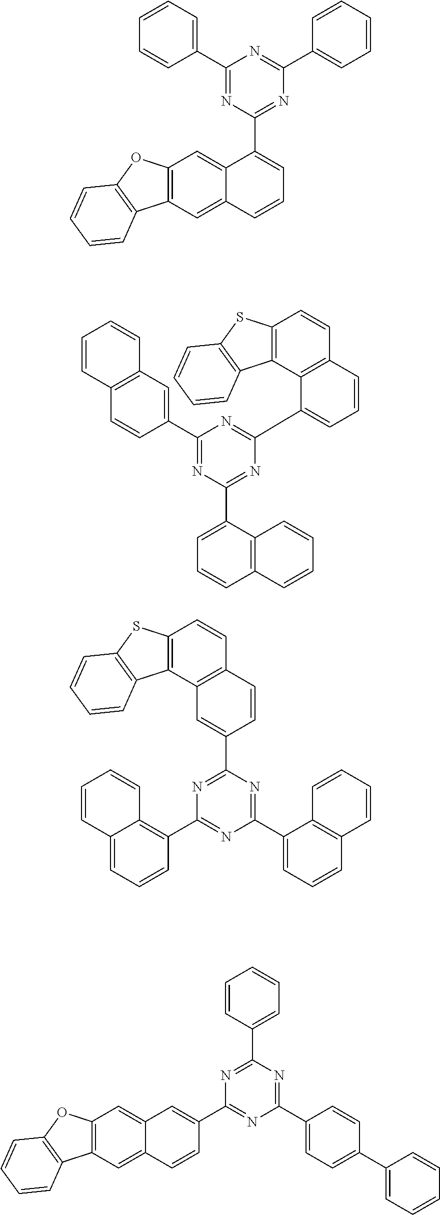

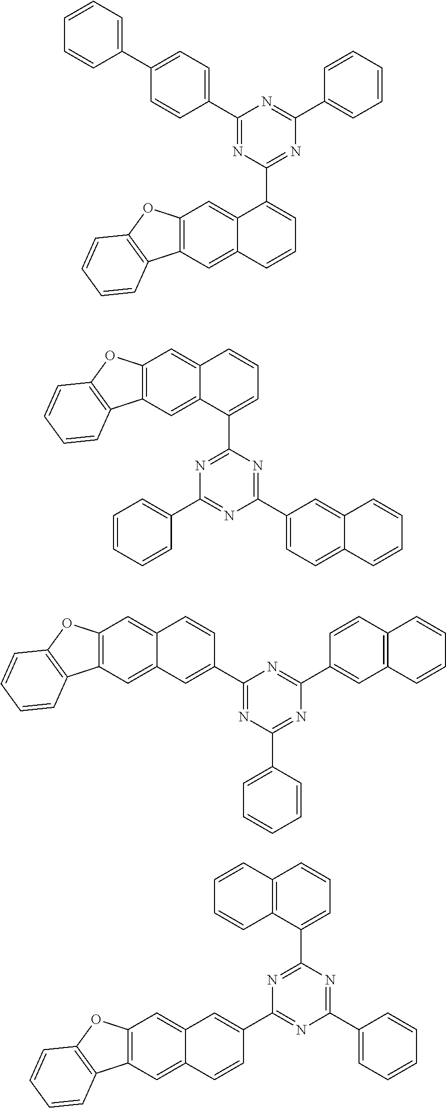

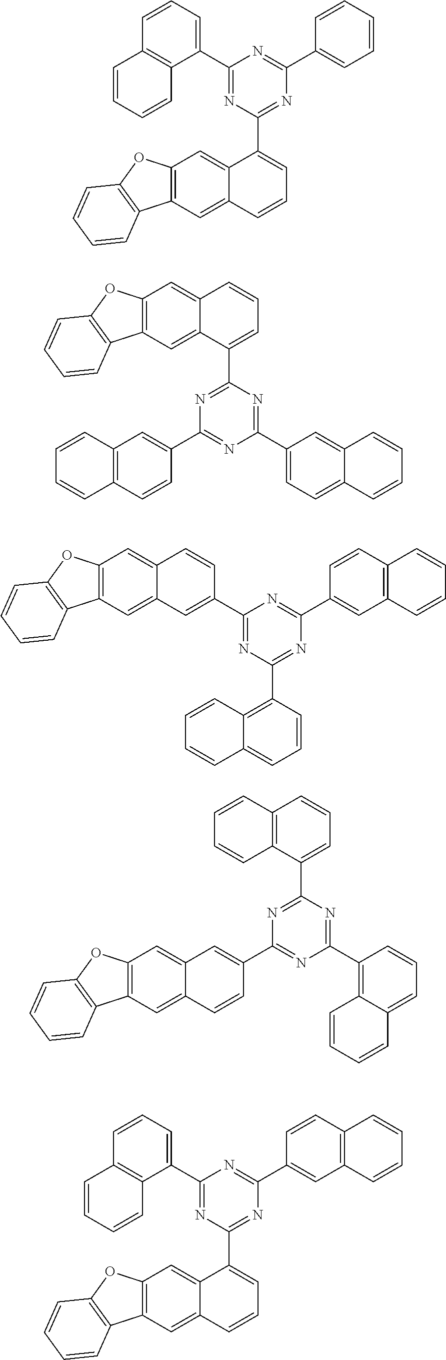

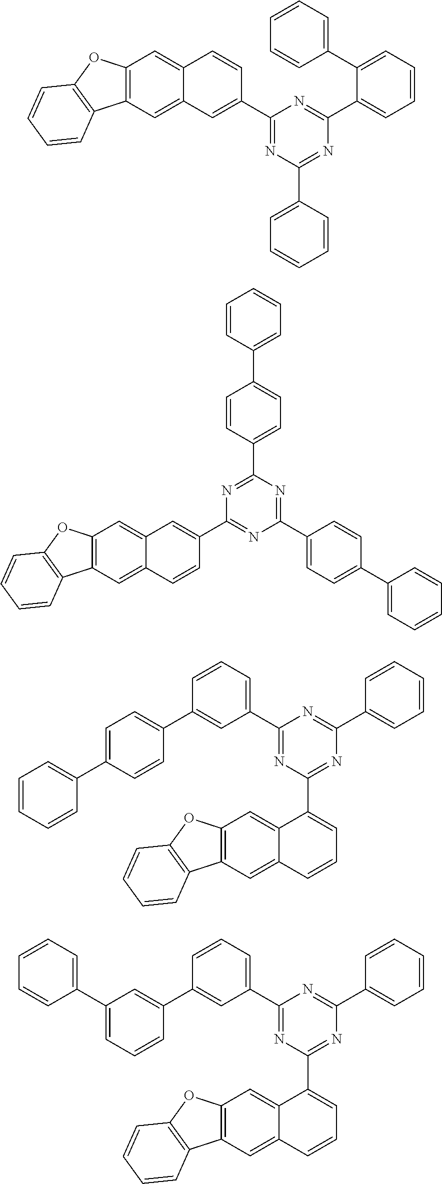

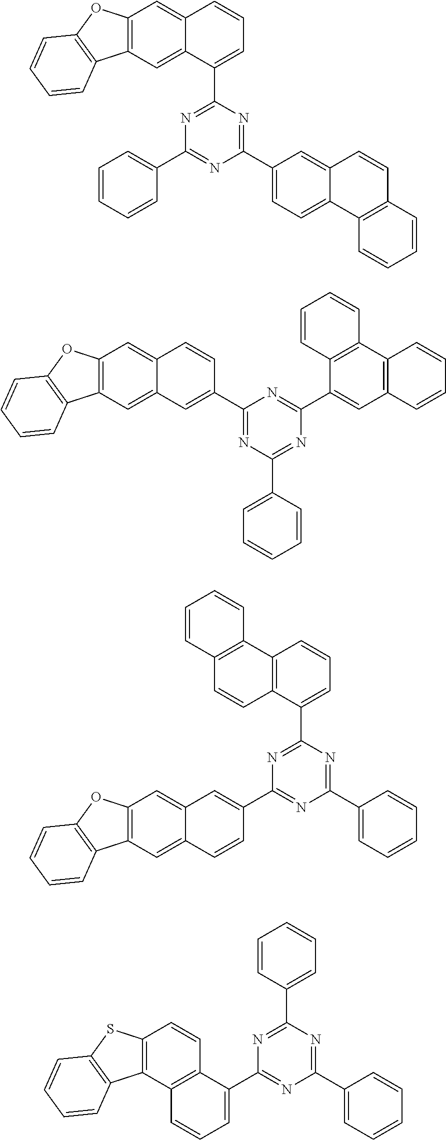 [SMILES: c1ccc(-c2nc(-c3ccc4c(ccc5ccccc54)c3)nc(-c3cccc4cc5oc6ccccc6c5cc34)n2)cc1 ; c1ccc(-c2nc(-c3ccc4cc5oc6ccccc6c5cc4c3)nc(-c3cc4ccccc4c4ccccc34)n2)cc1 ; c1ccc(-c2nc(-c3ccc4cc5c(cc4c3)oc3ccccc35)nc(-c3cccc4c3ccc3ccccc34)n2)cc1 ; c1ccc(-c2nc(-c3ccccc3)nc(-c3cccc4c3ccc3sc5ccccc5c34)n2)cc1]

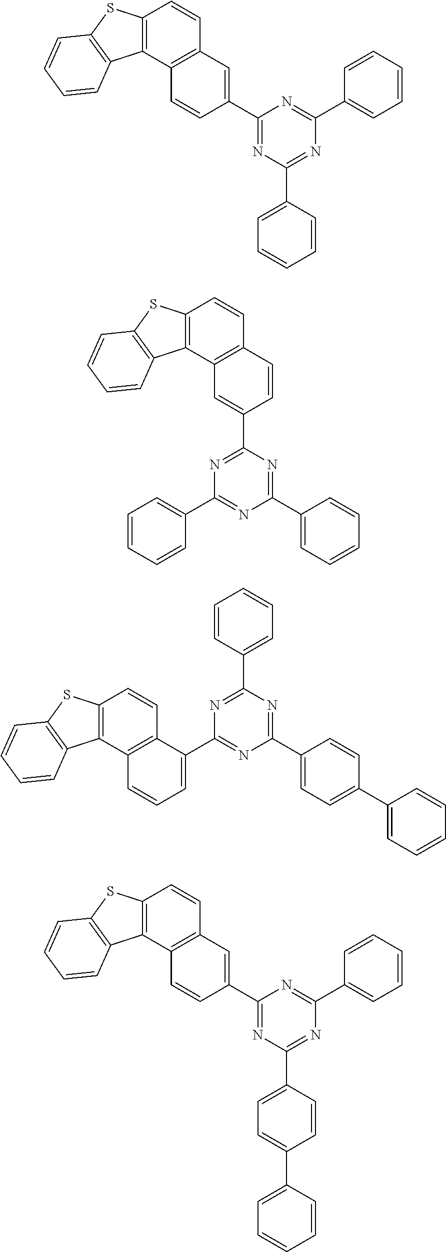 [SMILES: c1ccc(-c2nc(-c3ccccc3)nc(-c3ccc4c(ccc5sc6ccccc6c54)c3)n2)cc1 ; c1ccc(-c2nc(-c3ccccc3)nc(-c3ccc4ccc5sc6ccccc6c5c4c3)n2)cc1 ; c1ccc(-c2ccc(-c3nc(-c4ccccc4)nc(-c4cccc5c4ccc4sc6ccccc6c45)n3)cc2)cc1 ; c1ccc(-c2ccc(-c3nc(-c4ccccc4)nc(-c4ccc5c(ccc6sc7ccccc7c65)c4)n3)cc2)cc1]

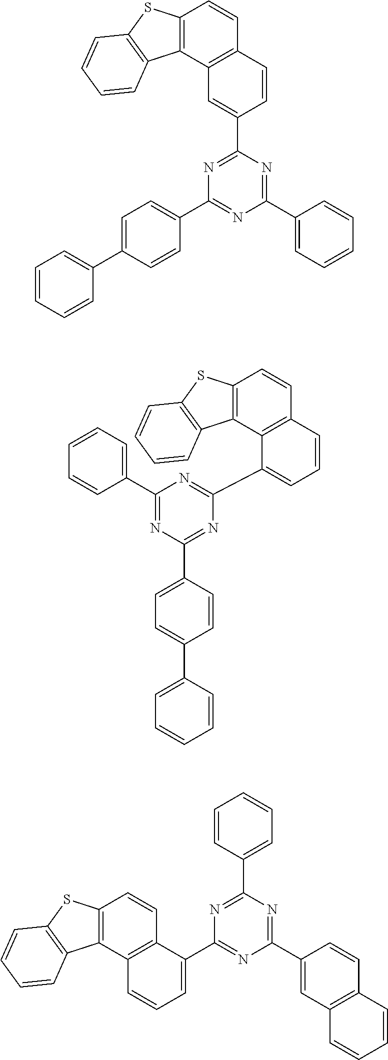 [SMILES: c1ccc(-c2ccc(-c3nc(-c4ccccc4)nc(-c4ccc5ccc6sc7ccccc7c6c5c4)n3)cc2)cc1 ; c1ccc(-c2ccc(-c3nc(-c4ccccc4)nc(-c4cccc5ccc6sc7ccccc7c6c45)n3)cc2)cc1 ; c1ccc(-c2nc(-c3ccc4ccccc4c3)nc(-c3cccc4c3ccc3sc5ccccc5c34)n2)cc1]

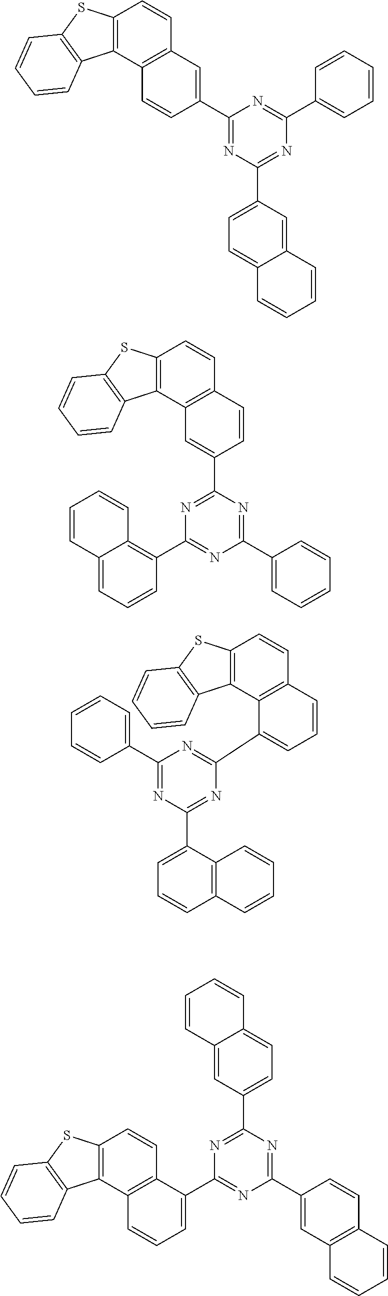

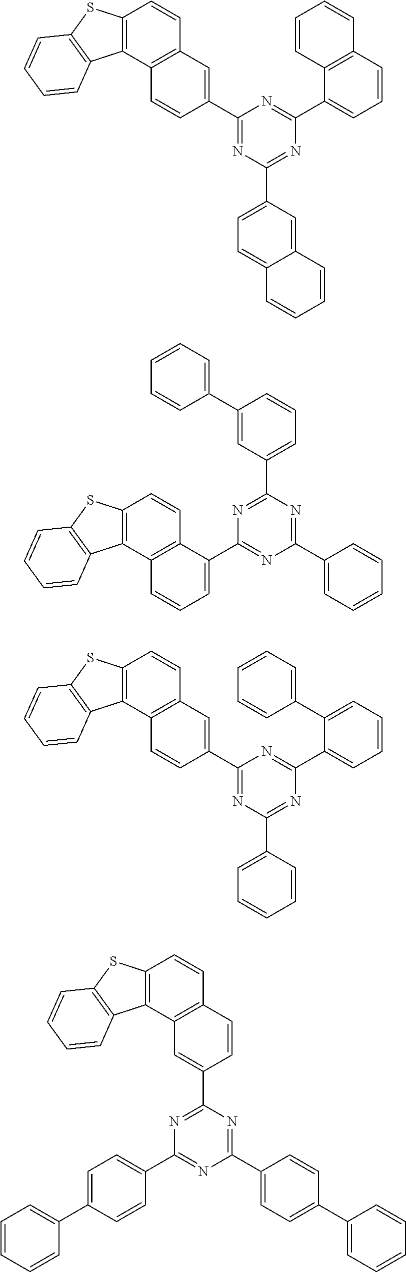 [SMILES: c1ccc2cc(-c3nc(-c4ccc5c(ccc6sc7ccccc7c65)c4)nc(-c4cccc5ccccc45)n3)ccc2c1 ; c1ccc(-c2cccc(-c3nc(-c4ccccc4)nc(-c4cccc5c4ccc4sc6ccccc6c45)n3)c2)cc1 ; c1ccc(-c2nc(-c3ccc4c(ccc5sc6ccccc6c54)c3)nc(-c3ccccc3-c3ccccc3)n2)cc1 ; c1ccc(-c2ccc(-c3nc(-c4ccc(-c5ccccc5)cc4)nc(-c4ccc5ccc6sc7ccccc7c6c5c4)n3)cc2)cc1]

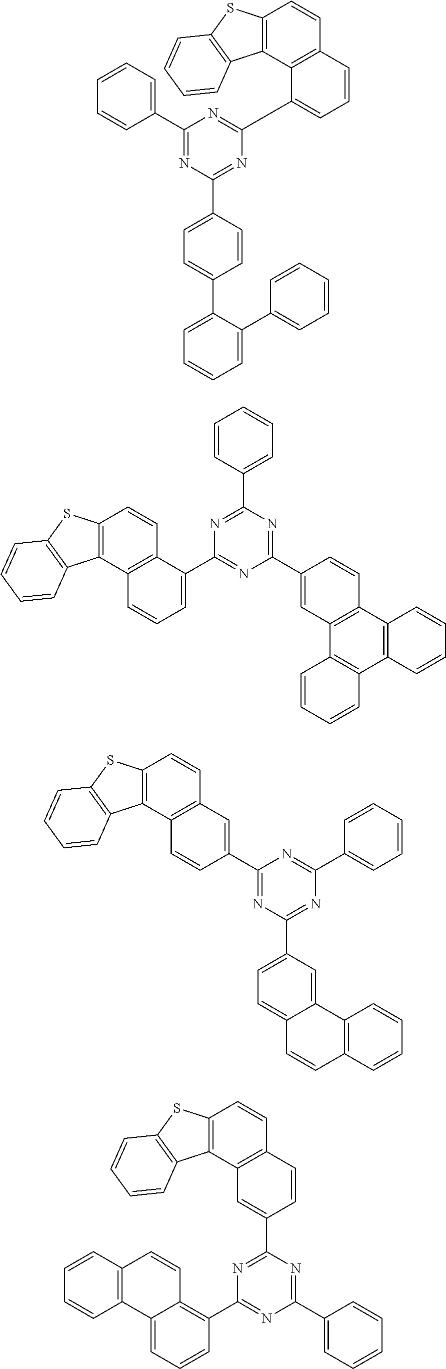

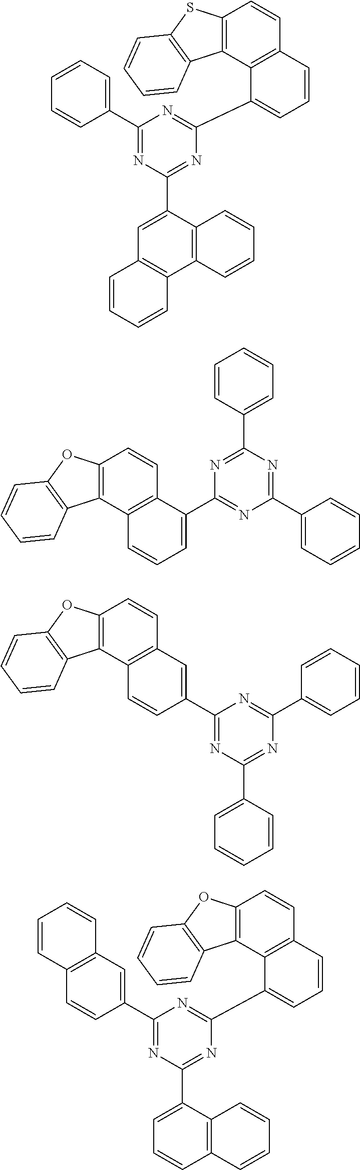

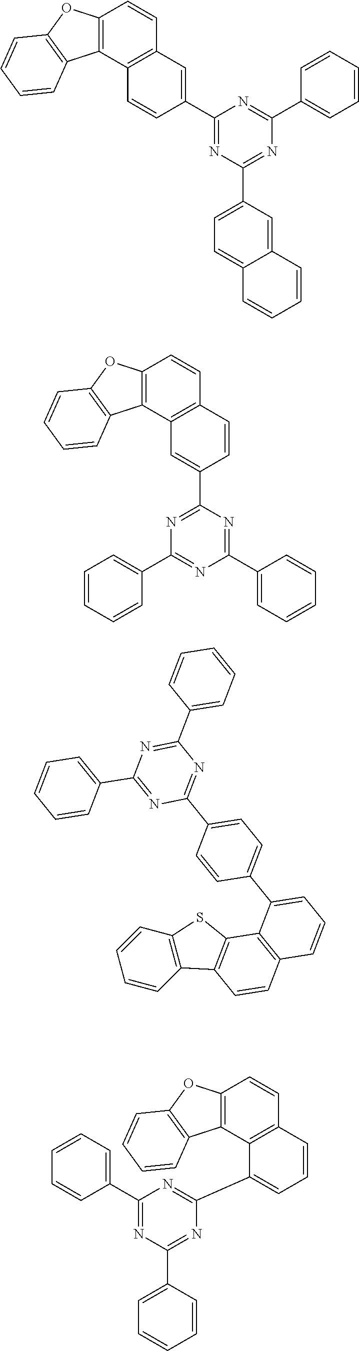 [SMILES: c1ccc(-c2nc(-c3ccc4ccccc4c3)nc(-c3ccc4c(ccc5oc6ccccc6c54)c3)n2)cc1 ; c1ccc(-c2nc(-c3ccccc3)nc(-c3ccc4ccc5oc6ccccc6c5c4c3)n2)cc1 ; c1ccc(-c2nc(-c3ccccc3)nc(-c3ccc(-c4cccc5ccc6c7ccccc7sc6c45)cc3)n2)cc1 ; c1ccc(-c2nc(-c3ccccc3)nc(-c3cccc4ccc5oc6ccccc6c5c34)n2)cc1]

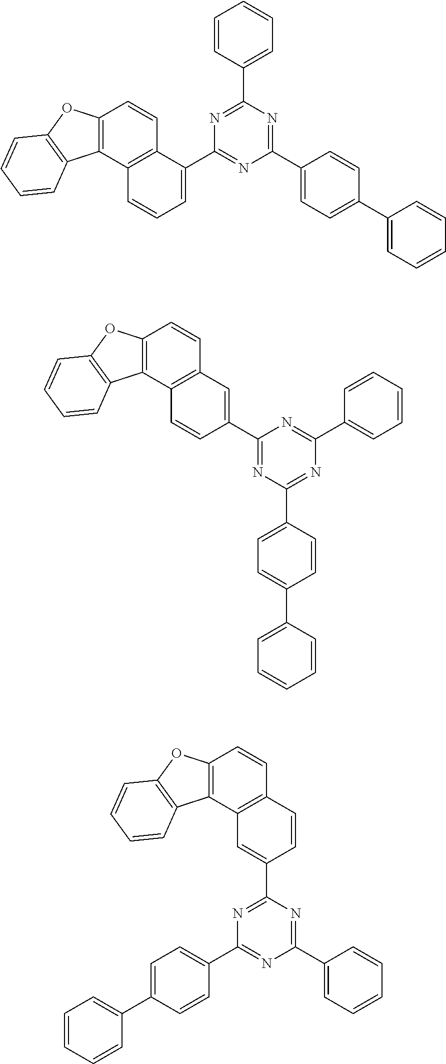 [SMILES: c1ccc(-c2ccc(-c3nc(-c4ccccc4)nc(-c4cccc5c4ccc4oc6ccccc6c45)n3)cc2)cc1 ; c1ccc(-c2ccc(-c3nc(-c4ccccc4)nc(-c4ccc5c(ccc6oc7ccccc7c65)c4)n3)cc2)cc1 ; c1ccc(-c2ccc(-c3nc(-c4ccccc4)nc(-c4ccc5ccc6oc7ccccc7c6c5c4)n3)cc2)cc1]

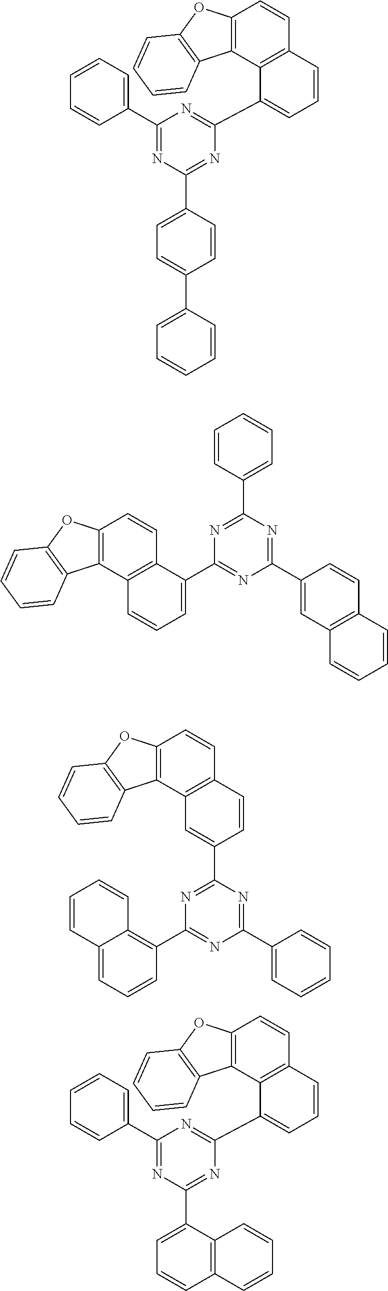

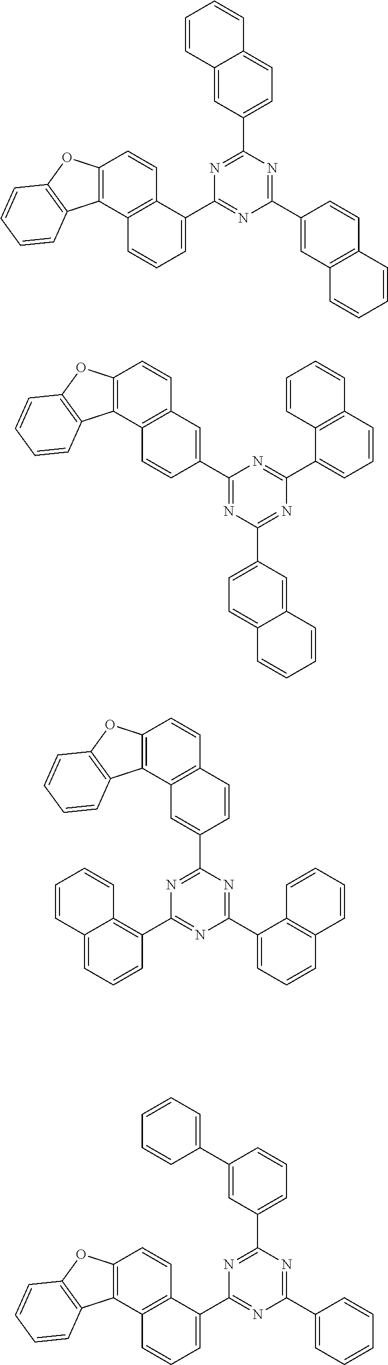 [SMILES: c1ccc2cc(-c3nc(-c4ccc5ccccc5c4)nc(-c4cccc5c4ccc4oc6ccccc6c45)n3)ccc2c1 ; c1ccc2cc(-c3nc(-c4ccc5c(ccc6oc7ccccc7c65)c4)nc(-c4cccc5ccccc45)n3)ccc2c1 ; c1ccc2c(-c3nc(-c4ccc5ccc6oc7ccccc7c6c5c4)nc(-c4cccc5ccccc45)n3)cccc2c1 ; c1ccc(-c2cccc(-c3nc(-c4ccccc4)nc(-c4cccc5c4ccc4oc6ccccc6c45)n3)c2)cc1]

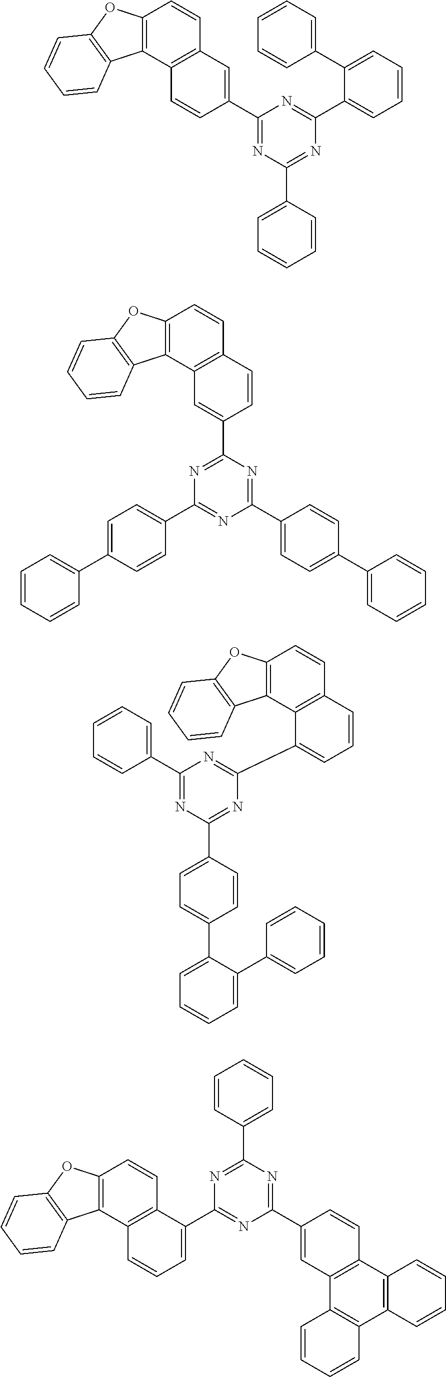 [SMILES: c1ccc(-c2nc(-c3ccc4c(ccc5oc6ccccc6c54)c3)nc(-c3ccccc3-c3ccccc3)n2)cc1 ; c1ccc(-c2ccc(-c3nc(-c4ccc(-c5ccccc5)cc4)nc(-c4ccc5ccc6oc7ccccc7c6c5c4)n3)cc2)cc1 ; c1ccc(-c2nc(-c3ccc(-c4ccccc4-c4ccccc4)cc3)nc(-c3cccc4ccc5oc6ccccc6c5c34)n2)cc1 ; c1ccc(-c2nc(-c3ccc4c5ccccc5c5ccccc5c4c3)nc(-c3cccc4c3ccc3oc5ccccc5c34)n2)cc1]

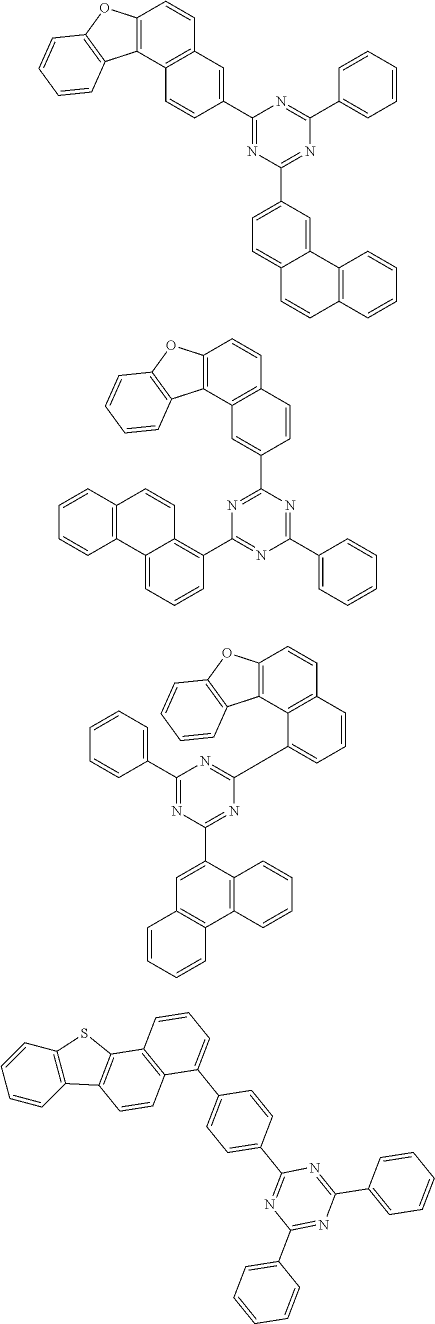

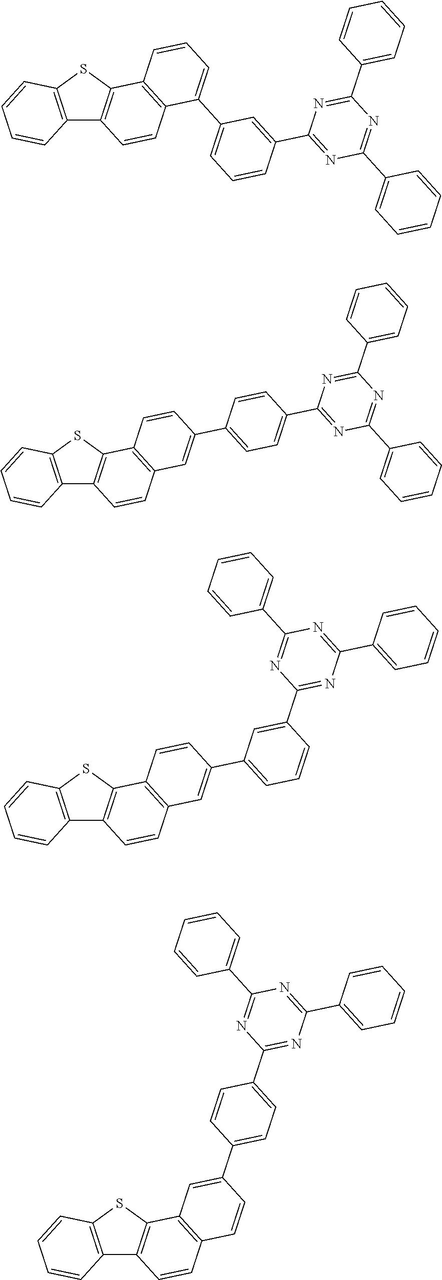 [SMILES: c1ccc(-c2nc(-c3ccccc3)nc(-c3cccc(-c4cccc5c4ccc4c6ccccc6sc54)c3)n2)cc1 ; c1ccc(-c2nc(-c3ccccc3)nc(-c3ccc(-c4ccc5c(ccc6c7ccccc7sc56)c4)cc3)n2)cc1 ; c1ccc(-c2nc(-c3ccccc3)nc(-c3cccc(-c4ccc5c(ccc6c7ccccc7sc56)c4)c3)n2)cc1 ; c1ccc(-c2nc(-c3ccccc3)nc(-c3ccc(-c4ccc5ccc6c7ccccc7sc6c5c4)cc3)n2)cc1]

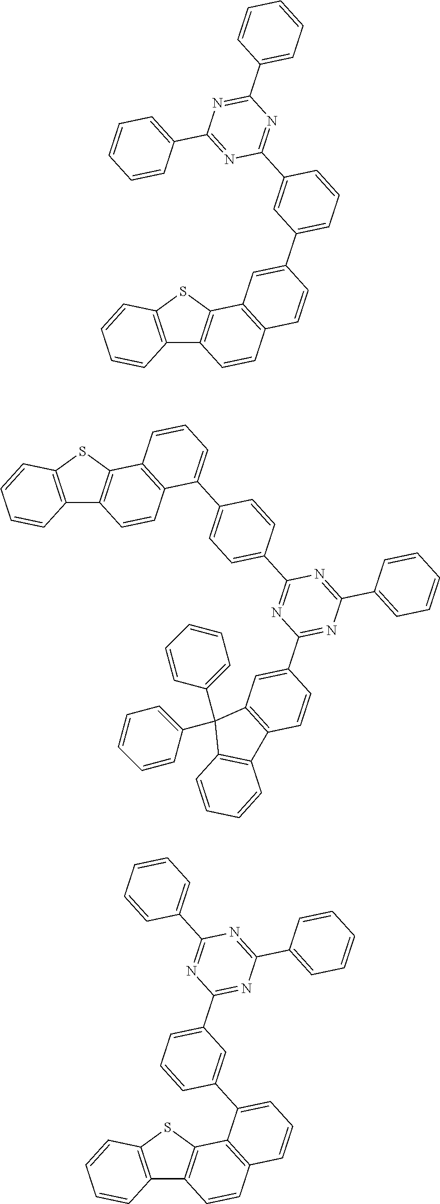 [SMILES: c1ccc(-c2nc(-c3ccccc3)nc(-c3cccc(-c4ccc5ccc6c7ccccc7sc6c5c4)c3)n2)cc1 ; c1ccc(-c2nc(-c3ccc(-c4cccc5c4ccc4c6ccccc6sc54)cc3)nc(-c3ccc4c(c3)C(c3ccccc3)(c3ccccc3)c3ccccc3-4)n2)cc1 ; c1ccc(-c2nc(-c3ccccc3)nc(-c3cccc(-c4cccc5ccc6c7ccccc7sc6c45)c3)n2)cc1]

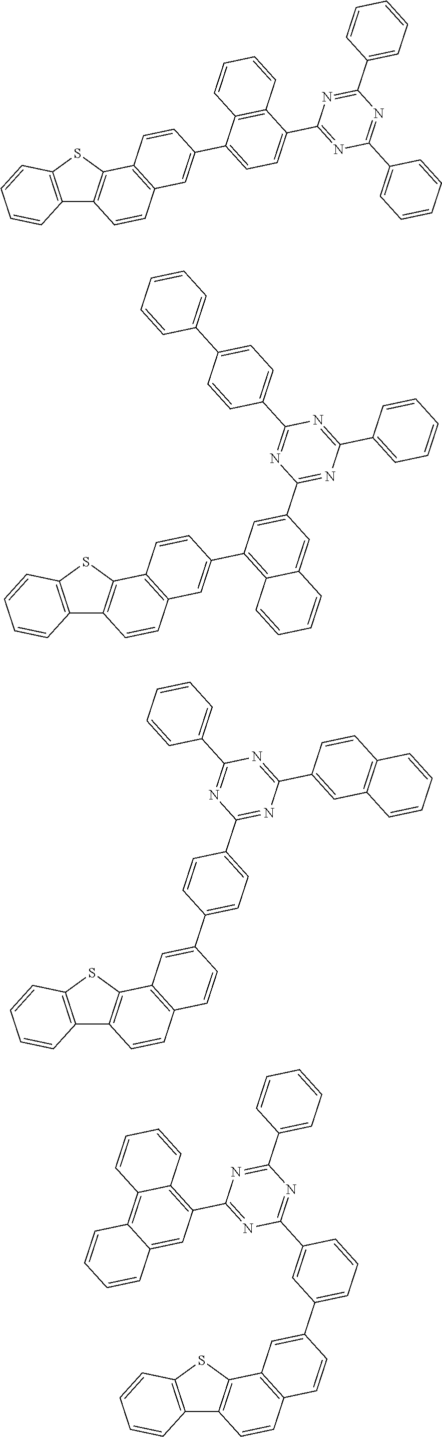 [SMILES: c1ccc(-c2nc(-c3ccccc3)nc(-c3ccc(-c4ccc5c(ccc6c7ccccc7sc56)c4)c4ccccc34)n2)cc1 ; c1ccc(-c2ccc(-c3nc(-c4ccccc4)nc(-c4cc(-c5ccc6c(ccc7c8ccccc8sc67)c5)c5ccccc5c4)n3)cc2)cc1 ; c1ccc(-c2nc(-c3ccc(-c4ccc5ccc6c7ccccc7sc6c5c4)cc3)nc(-c3ccc4ccccc4c3)n2)cc1 ; c1ccc(-c2nc(-c3cccc(-c4ccc5ccc6c7ccccc7sc6c5c4)c3)nc(-c3cc4ccccc4c4ccccc34)n2)cc1]

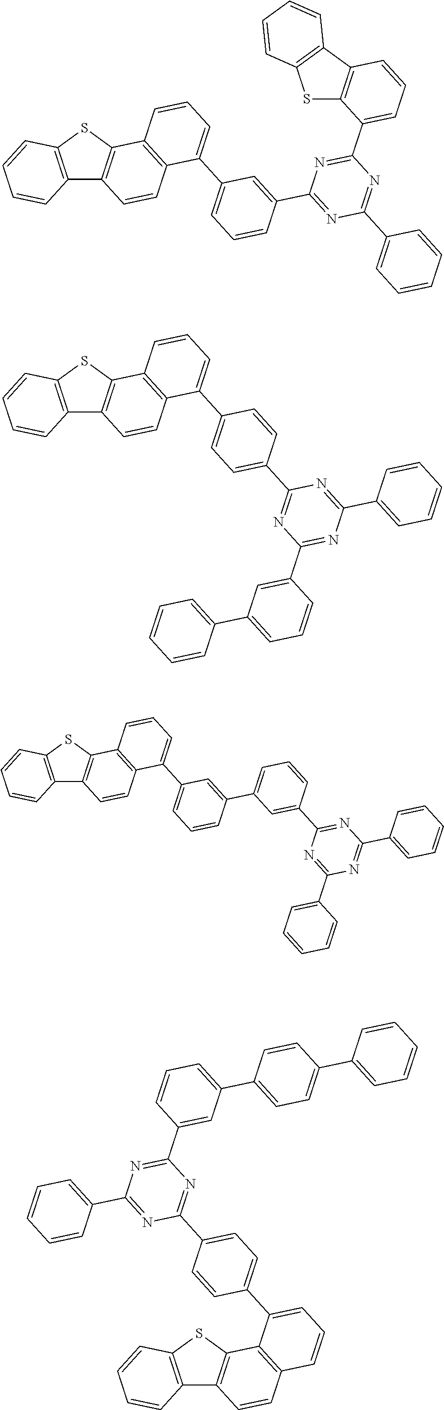 [SMILES: c1ccc(-c2nc(-c3cccc(-c4cccc5c4ccc4c6ccccc6sc54)c3)nc(-c3cccc4c3sc3ccccc34)n2)cc1 ; c1ccc(-c2cccc(-c3nc(-c4ccccc4)nc(-c4ccc(-c5cccc6c5ccc5c7ccccc7sc65)cc4)n3)c2)cc1 ; c1ccc(-c2nc(-c3ccccc3)nc(-c3cccc(-c4cccc(-c5cccc6c5ccc5c7ccccc7sc65)c4)c3)n2)cc1 ; c1ccc(-c2ccc(-c3cccc(-c4nc(-c5ccccc5)nc(-c5ccc(-c6cccc7ccc8c9ccccc9sc8c67)cc5)n4)c3)cc2)cc1]

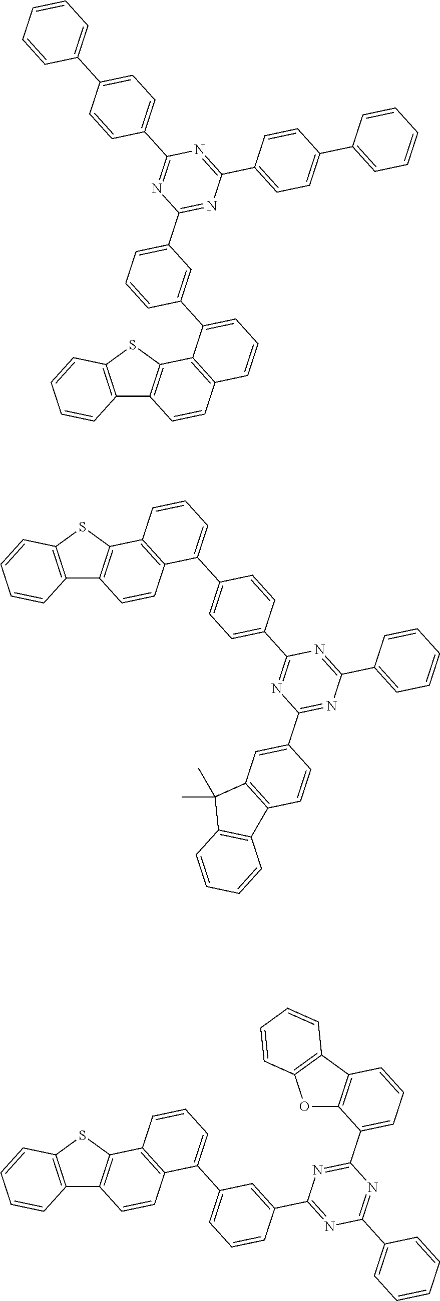 [SMILES: c1ccc(-c2ccc(-c3nc(-c4ccc(-c5ccccc5)cc4)nc(-c4cccc(-c5cccc6ccc7c8ccccc8sc7c56)c4)n3)cc2)cc1 ; CC1(C)c2ccccc2-c2ccc(-c3nc(-c4ccccc4)nc(-c4ccc(-c5cccc6c5ccc5c7ccccc7sc65)cc4)n3)cc21 ; c1ccc(-c2nc(-c3cccc(-c4cccc5c4ccc4c6ccccc6sc54)c3)nc(-c3cccc4c3oc3ccccc34)n2)cc1]

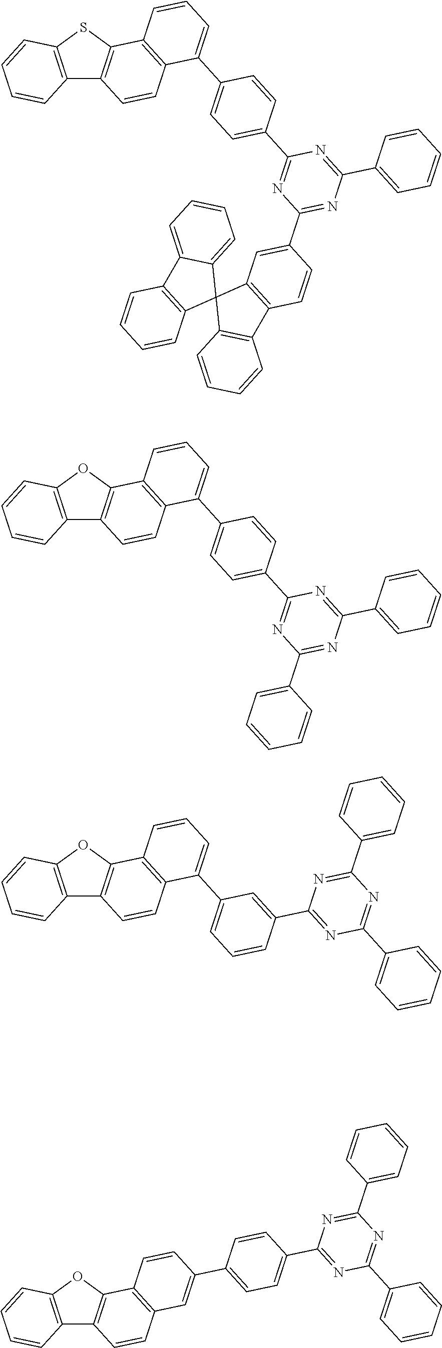 [SMILES: c1ccc(-c2nc(-c3ccc(-c4cccc5c4ccc4c6ccccc6sc54)cc3)nc(-c3ccc4c(c3)C3(c5ccccc5-c5ccccc53)c3ccccc3-4)n2)cc1 ; c1ccc(-c2nc(-c3ccccc3)nc(-c3ccc(-c4cccc5c4ccc4c6ccccc6oc54)cc3)n2)cc1 ; c1ccc(-c2nc(-c3ccccc3)nc(-c3cccc(-c4cccc5c4ccc4c6ccccc6oc54)c3)n2)cc1 ; c1ccc(-c2nc(-c3ccccc3)nc(-c3ccc(-c4ccc5c(ccc6c7ccccc7oc56)c4)cc3)n2)cc1]

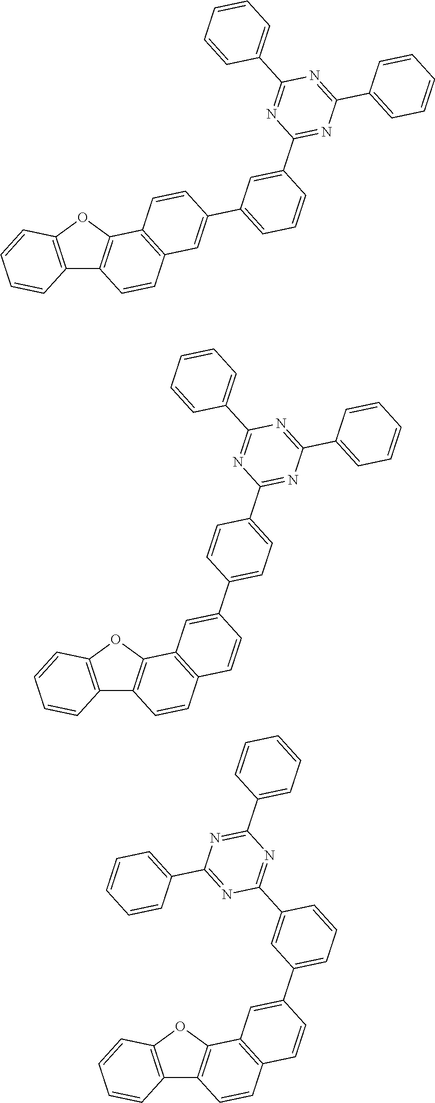 [SMILES: c1ccc(-c2nc(-c3ccccc3)nc(-c3cccc(-c4ccc5c(ccc6c7ccccc7oc56)c4)c3)n2)cc1 ; c1ccc(-c2nc(-c3ccccc3)nc(-c3ccc(-c4ccc5ccc6c7ccccc7oc6c5c4)cc3)n2)cc1 ; c1ccc(-c2nc(-c3ccccc3)nc(-c3cccc(-c4ccc5ccc6c7ccccc7oc6c5c4)c3)n2)cc1]

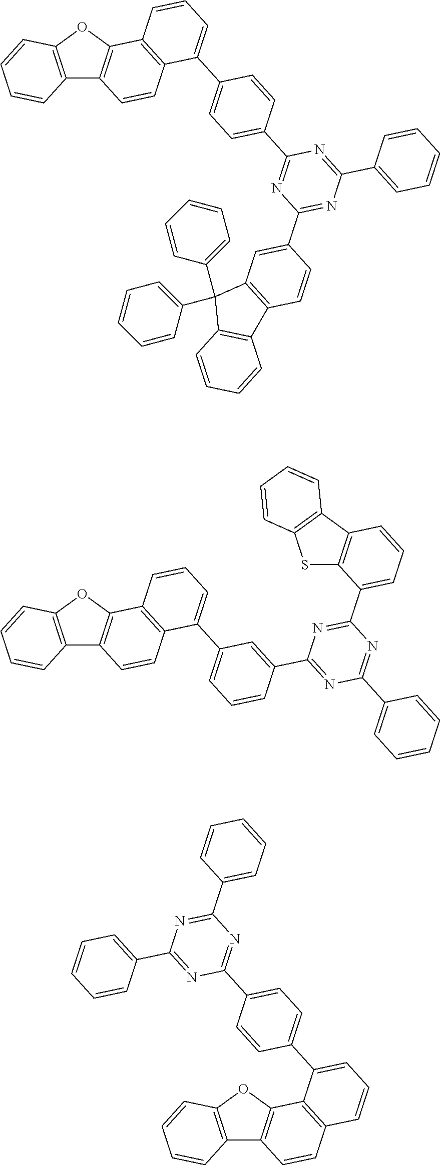 [SMILES: c1ccc(-c2nc(-c3ccc(-c4cccc5c4ccc4c6ccccc6oc54)cc3)nc(-c3ccc4c(c3)C(c3ccccc3)(c3ccccc3)c3ccccc3-4)n2)cc1 ; c1ccc(-c2nc(-c3cccc(-c4cccc5c4ccc4c6ccccc6oc54)c3)nc(-c3cccc4c3sc3ccccc34)n2)cc1 ; c1ccc(-c2nc(-c3ccccc3)nc(-c3ccc(-c4cccc5ccc6c7ccccc7oc6c45)cc3)n2)cc1]

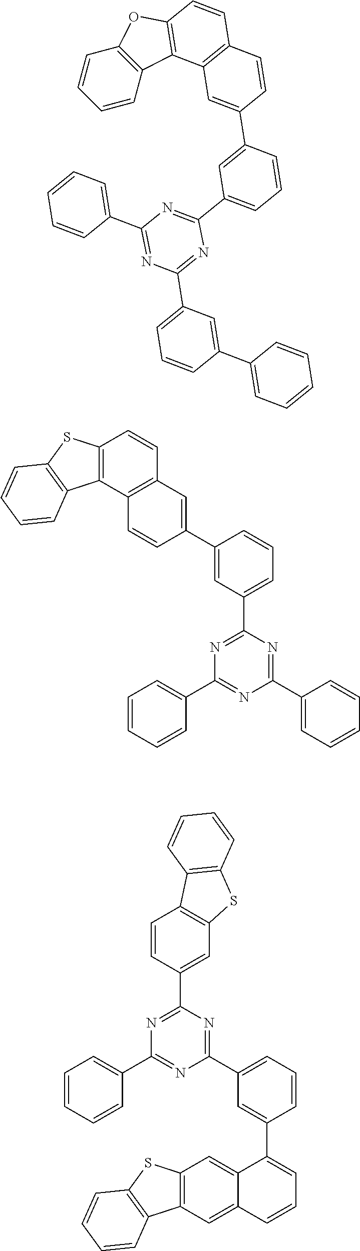 [SMILES: c1ccc(-c2cccc(-c3nc(-c4ccccc4)nc(-c4cccc(-c5ccc6ccc7oc8ccccc8c7c6c5)c4)n3)c2)cc1 ; c1ccc(-c2nc(-c3ccccc3)nc(-c3cccc(-c4ccc5c(ccc6sc7ccccc7c65)c4)c3)n2)cc1 ; c1ccc(-c2nc(-c3cccc(-c4cccc5cc6c(cc45)sc4ccccc46)c3)nc(-c3ccc4c(c3)sc3ccccc34)n2)cc1]

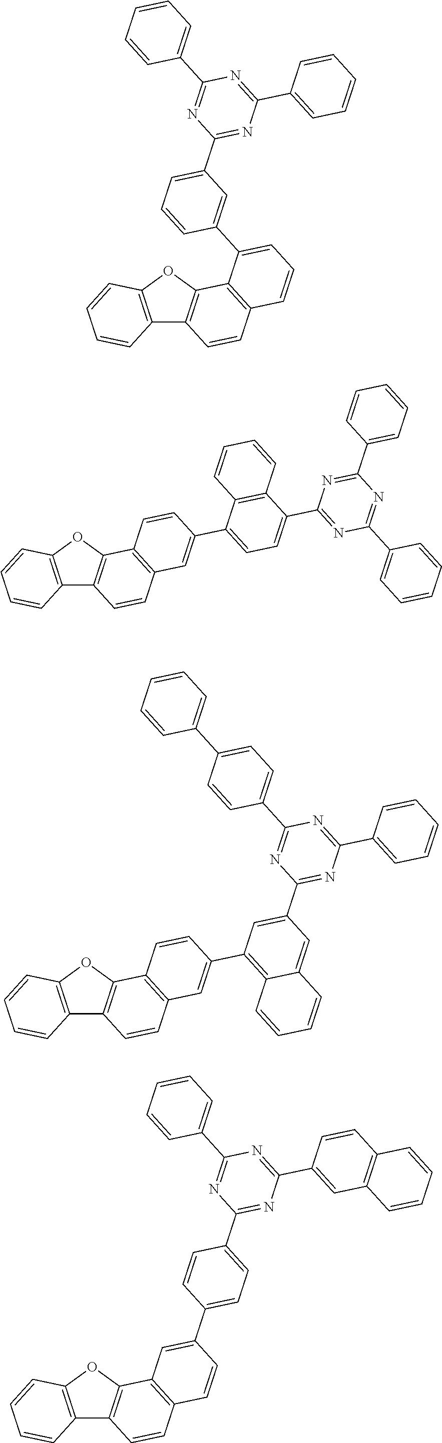 [SMILES: c1ccc(-c2nc(-c3ccccc3)nc(-c3cccc(-c4cccc5ccc6c7ccccc7oc6c45)c3)n2)cc1 ; c1ccc(-c2nc(-c3ccccc3)nc(-c3ccc(-c4ccc5c(ccc6c7ccccc7oc56)c4)c4ccccc34)n2)cc1 ; c1ccc(-c2ccc(-c3nc(-c4ccccc4)nc(-c4cc(-c5ccc6c(ccc7c8ccccc8oc67)c5)c5ccccc5c4)n3)cc2)cc1 ; c1ccc(-c2nc(-c3ccc(-c4ccc5ccc6c7ccccc7oc6c5c4)cc3)nc(-c3ccc4ccccc4c3)n2)cc1]

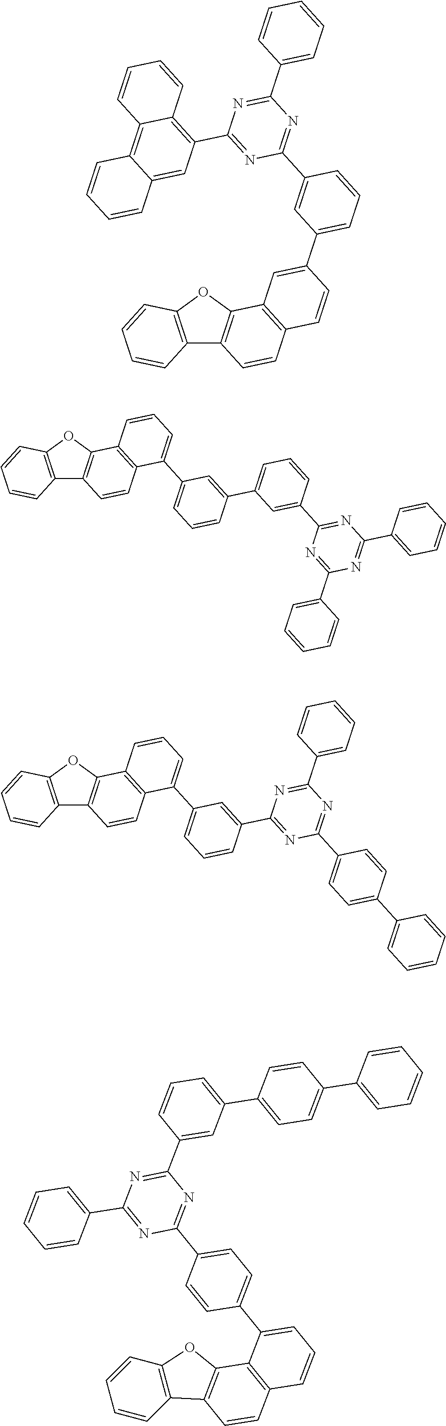 [SMILES: c1ccc(-c2nc(-c3cccc(-c4ccc5ccc6c7ccccc7oc6c5c4)c3)nc(-c3cc4ccccc4c4ccccc34)n2)cc1 ; c1ccc(-c2nc(-c3ccccc3)nc(-c3cccc(-c4cccc(-c5cccc6c5ccc5c7ccccc7oc65)c4)c3)n2)cc1 ; c1ccc(-c2ccc(-c3nc(-c4ccccc4)nc(-c4cccc(-c5cccc6c5ccc5c7ccccc7oc65)c4)n3)cc2)cc1 ; c1ccc(-c2ccc(-c3cccc(-c4nc(-c5ccccc5)nc(-c5ccc(-c6cccc7ccc8c9ccccc9oc8c67)cc5)n4)c3)cc2)cc1]

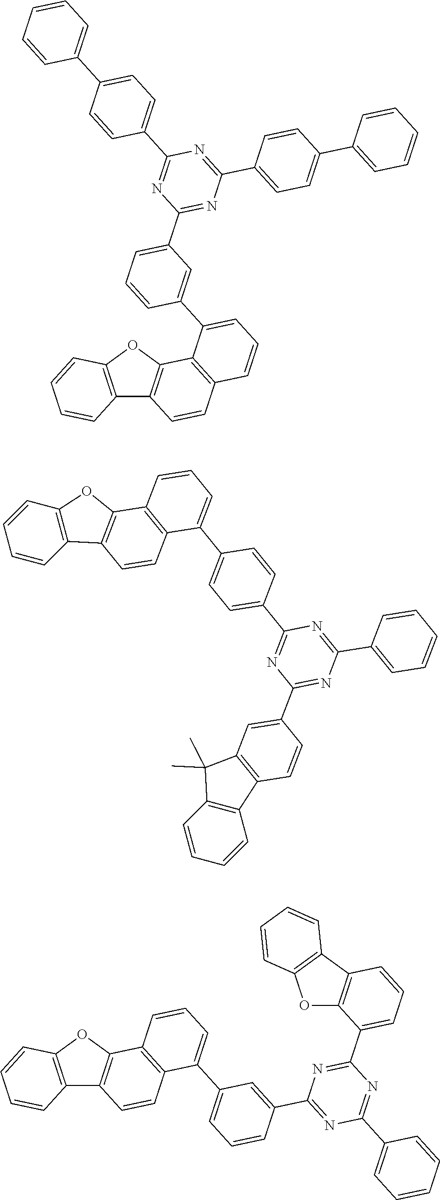 [SMILES: c1ccc(-c2ccc(-c3nc(-c4ccc(-c5ccccc5)cc4)nc(-c4cccc(-c5cccc6ccc7c8ccccc8oc7c56)c4)n3)cc2)cc1 ; CC1(C)c2ccccc2-c2ccc(-c3nc(-c4ccccc4)nc(-c4ccc(-c5cccc6c5ccc5c7ccccc7oc65)cc4)n3)cc21 ; c1ccc(-c2nc(-c3cccc(-c4cccc5c4ccc4c6ccccc6oc54)c3)nc(-c3cccc4c3oc3ccccc34)n2)cc1]

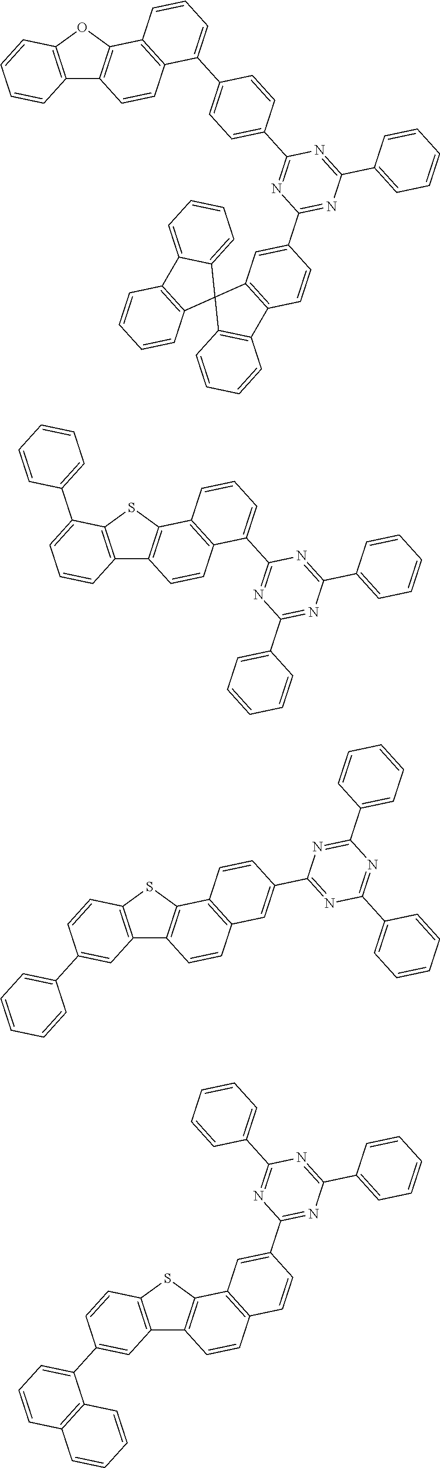 [SMILES: c1ccc(-c2nc(-c3ccc(-c4cccc5c4ccc4c6ccccc6oc54)cc3)nc(-c3ccc4c(c3)C3(c5ccccc5-c5ccccc53)c3ccccc3-4)n2)cc1 ; c1ccc(-c2nc(-c3ccccc3)nc(-c3cccc4c3ccc3c5cccc(-c6ccccc6)c5sc43)n2)cc1 ; c1ccc(-c2ccc3sc4c5ccc(-c6nc(-c7ccccc7)nc(-c7ccccc7)n6)cc5ccc4c3c2)cc1 ; c1ccc(-c2nc(-c3ccccc3)nc(-c3ccc4ccc5c6cc(-c7cccc8ccccc78)ccc6sc5c4c3)n2)cc1]

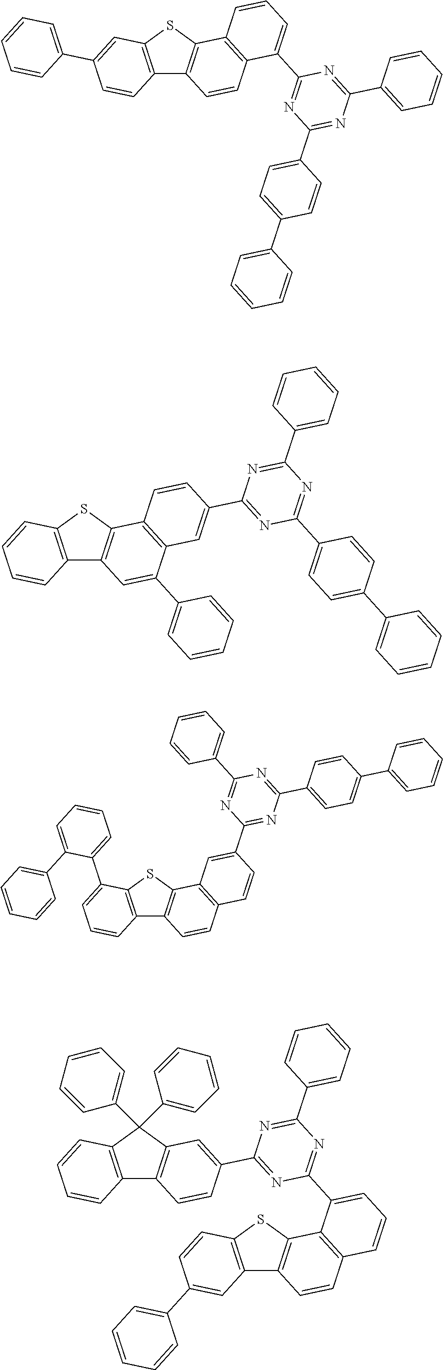 [SMILES: c1ccc(-c2ccc(-c3nc(-c4ccccc4)nc(-c4cccc5c4ccc4c6ccc(-c7ccccc7)cc6sc54)n3)cc2)cc1 ; c1ccc(-c2ccc(-c3nc(-c4ccccc4)nc(-c4ccc5c(c4)c(-c4ccccc4)cc4c6ccccc6sc54)n3)cc2)cc1 ; c1ccc(-c2ccc(-c3nc(-c4ccccc4)nc(-c4ccc5ccc6c7cccc(-c8ccccc8-c8ccccc8)c7sc6c5c4)n3)cc2)cc1 ; c1ccc(-c2ccc3sc4c(ccc5cccc(-c6nc(-c7ccccc7)nc(-c7ccc8c(c7)C(c7ccccc7)(c7ccccc7)c7ccccc7-8)n6)c54)c3c2)cc1]

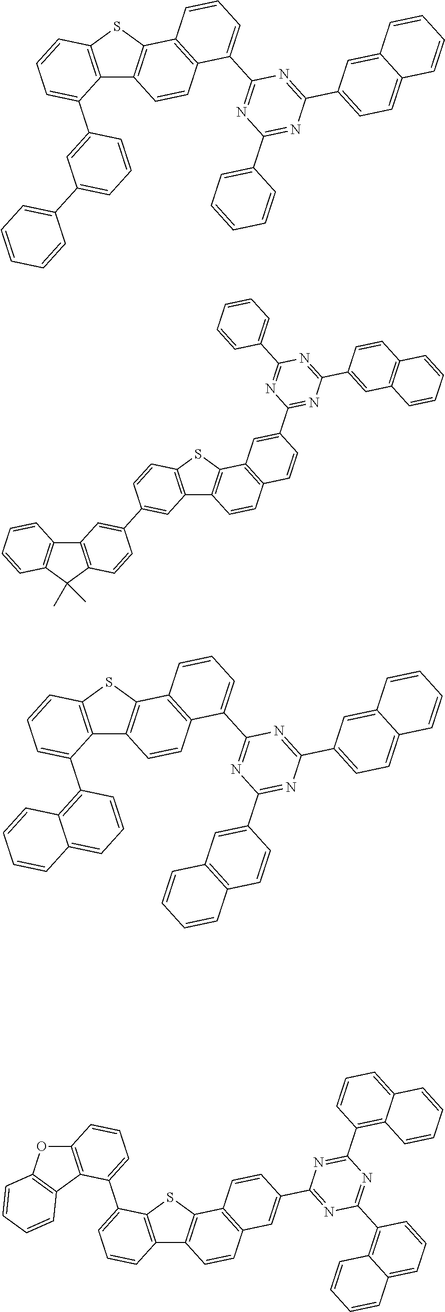 [SMILES: c1ccc(-c2cccc(-c3cccc4sc5c6cccc(-c7nc(-c8ccccc8)nc(-c8ccc9ccccc9c8)n7)c6ccc5c34)c2)cc1 ; CC1(C)c2ccccc2-c2cc(-c3ccc4sc5c6cc(-c7nc(-c8ccccc8)nc(-c8ccc9ccccc9c8)n7)ccc6ccc5c4c3)ccc21 ; c1ccc2cc(-c3nc(-c4ccc5ccccc5c4)nc(-c4cccc5c4ccc4c5sc5cccc(-c6cccc7ccccc67)c54)n3)ccc2c1 ; c1ccc2c(-c3nc(-c4ccc5c(ccc6c7cccc(-c8cccc9oc%10ccccc%10c89)c7sc56)c4)nc(-c4cccc5ccccc45)n3)cccc2c1]

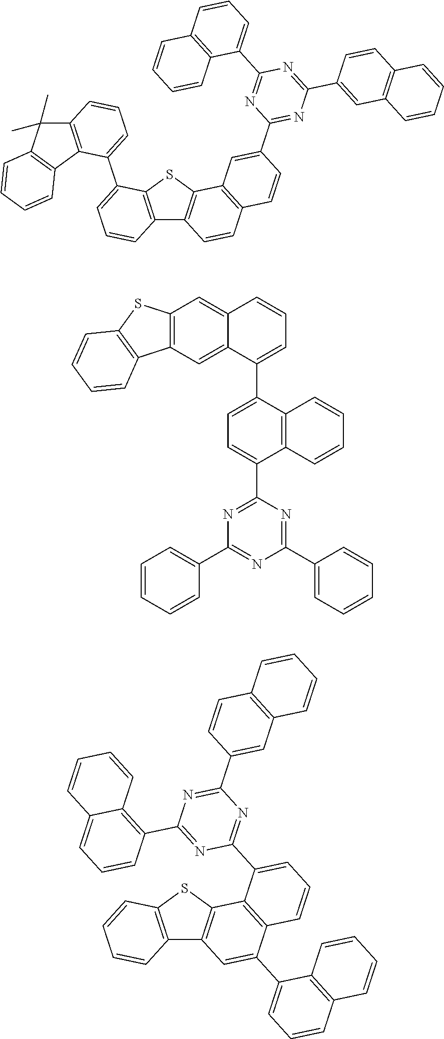 [SMILES: CC1(C)c2ccccc2-c2c(-c3cccc4c3sc3c5cc(-c6nc(-c7ccc8ccccc8c7)nc(-c7cccc8ccccc78)n6)ccc5ccc43)cccc21 ; c1ccc(-c2nc(-c3ccccc3)nc(-c3ccc(-c4cccc5cc6sc7ccccc7c6cc45)c4ccccc34)n2)cc1 ; c1ccc2cc(-c3nc(-c4cccc5ccccc45)nc(-c4cccc5c(-c6cccc7ccccc67)cc6c7ccccc7sc6c45)n3)ccc2c1]

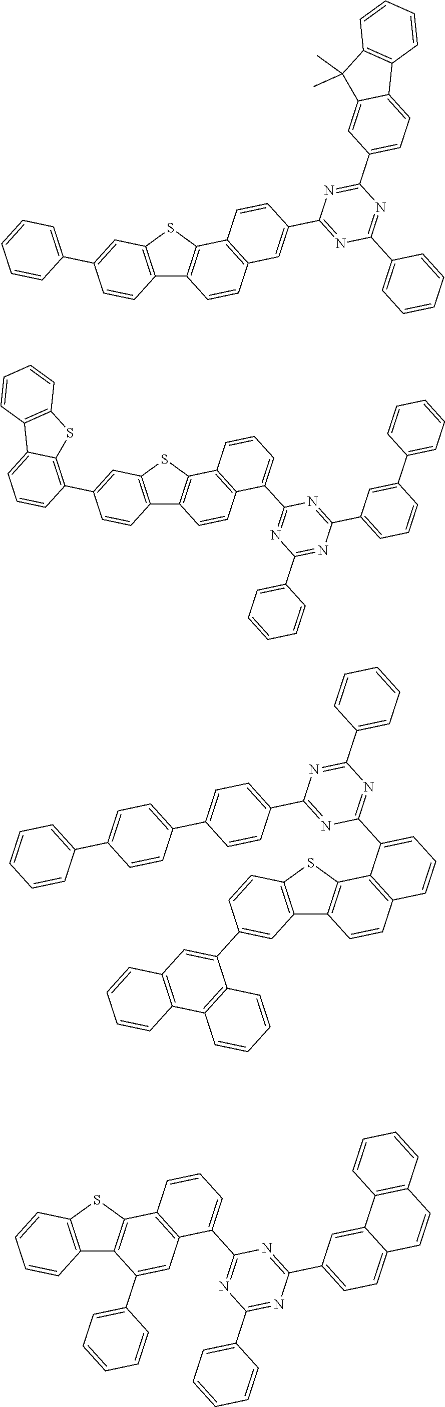 [SMILES: CC1(C)c2ccccc2-c2ccc(-c3nc(-c4ccccc4)nc(-c4ccc5c(ccc6c7ccc(-c8ccccc8)cc7sc56)c4)n3)cc21 ; c1ccc(-c2cccc(-c3nc(-c4ccccc4)nc(-c4cccc5c4ccc4c6ccc(-c7cccc8c7sc7ccccc78)cc6sc54)n3)c2)cc1 ; c1ccc(-c2ccc(-c3ccc(-c4nc(-c5ccccc5)nc(-c5cccc6ccc7c8cc(-c9cc%10ccccc%10c%10ccccc9%10)ccc8sc7c56)n4)cc3)cc2)cc1 ; c1ccc(-c2nc(-c3ccc4ccc5ccccc5c4c3)nc(-c3cccc4c3cc(-c3ccccc3)c3c5ccccc5sc43)n2)cc1]

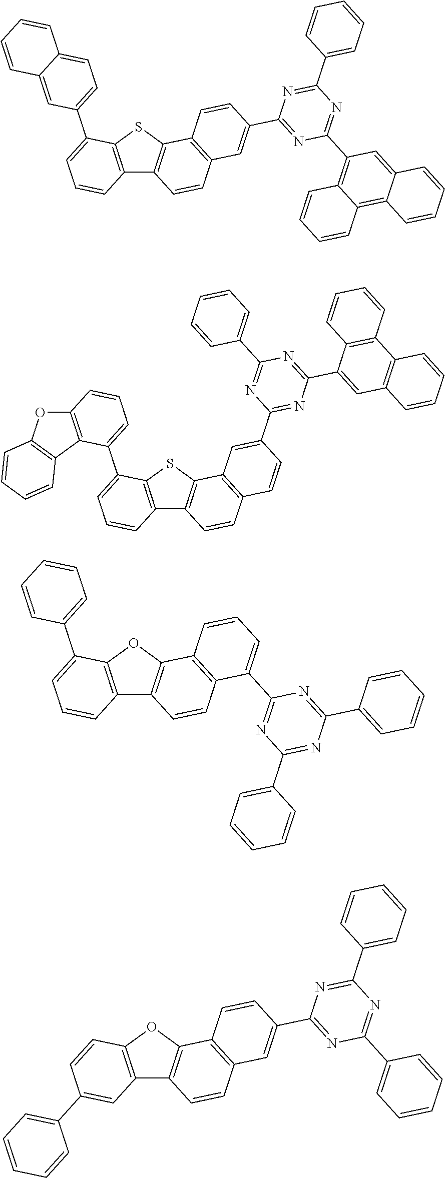 [SMILES: c1ccc(-c2nc(-c3ccc4c(ccc5c6cccc(-c7ccc8ccccc8c7)c6sc45)c3)nc(-c3cc4ccccc4c4ccccc34)n2)cc1 ; c1ccc(-c2nc(-c3ccc4ccc5c6cccc(-c7cccc8oc9ccccc9c78)c6sc5c4c3)nc(-c3cc4ccccc4c4ccccc34)n2)cc1 ; c1ccc(-c2nc(-c3ccccc3)nc(-c3cccc4c3ccc3c5cccc(-c6ccccc6)c5oc43)n2)cc1 ; c1ccc(-c2ccc3oc4c5ccc(-c6nc(-c7ccccc7)nc(-c7ccccc7)n6)cc5ccc4c3c2)cc1]

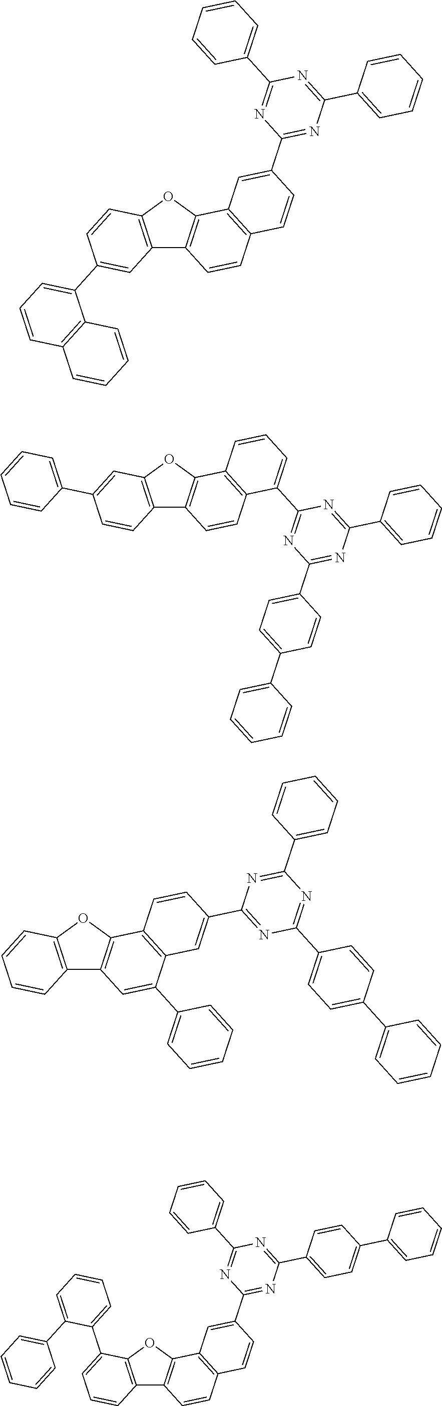 [SMILES: c1ccc(-c2nc(-c3ccccc3)nc(-c3ccc4ccc5c6cc(-c7cccc8ccccc78)ccc6oc5c4c3)n2)cc1 ; c1ccc(-c2ccc(-c3nc(-c4ccccc4)nc(-c4cccc5c4ccc4c6ccc(-c7ccccc7)cc6oc54)n3)cc2)cc1 ; c1ccc(-c2ccc(-c3nc(-c4ccccc4)nc(-c4ccc5c(c4)c(-c4ccccc4)cc4c6ccccc6oc54)n3)cc2)cc1 ; c1ccc(-c2ccc(-c3nc(-c4ccccc4)nc(-c4ccc5ccc6c7cccc(-c8ccccc8-c8ccccc8)c7oc6c5c4)n3)cc2)cc1]

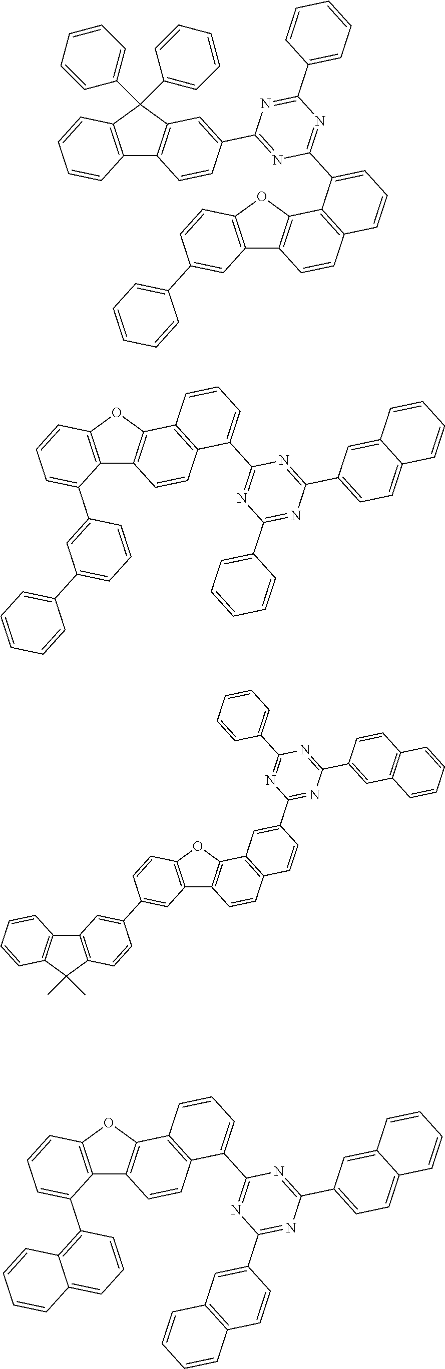 [SMILES: c1ccc(-c2ccc3oc4c(ccc5cccc(-c6nc(-c7ccccc7)nc(-c7ccc8c(c7)C(c7ccccc7)(c7ccccc7)c7ccccc7-8)n6)c54)c3c2)cc1 ; c1ccc(-c2cccc(-c3cccc4oc5c6cccc(-c7nc(-c8ccccc8)nc(-c8ccc9ccccc9c8)n7)c6ccc5c34)c2)cc1 ; CC1(C)c2ccccc2-c2cc(-c3ccc4oc5c6cc(-c7nc(-c8ccccc8)nc(-c8ccc9ccccc9c8)n7)ccc6ccc5c4c3)ccc21 ; c1ccc2cc(-c3nc(-c4ccc5ccccc5c4)nc(-c4cccc5c4ccc4c5oc5cccc(-c6cccc7ccccc67)c54)n3)ccc2c1]

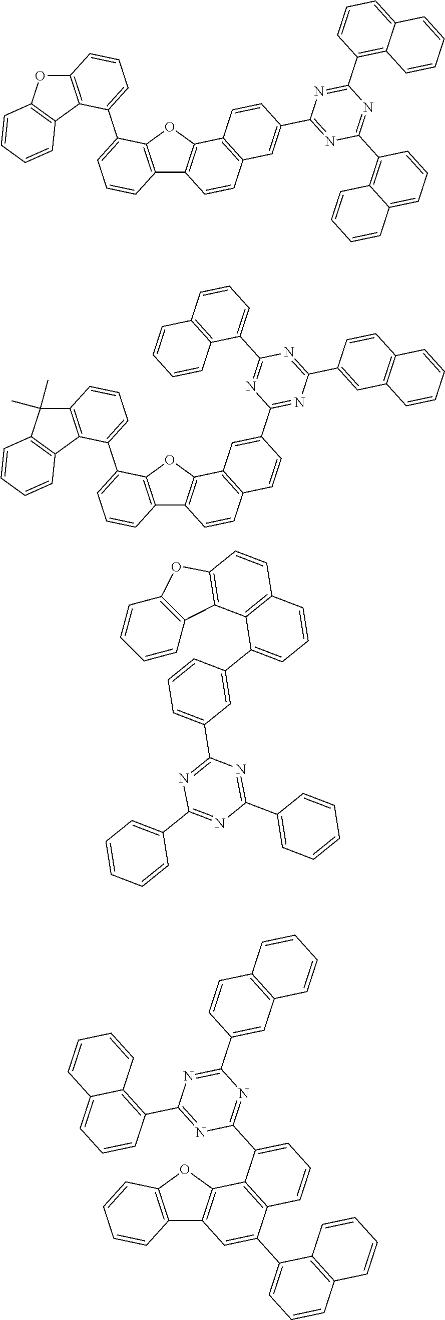

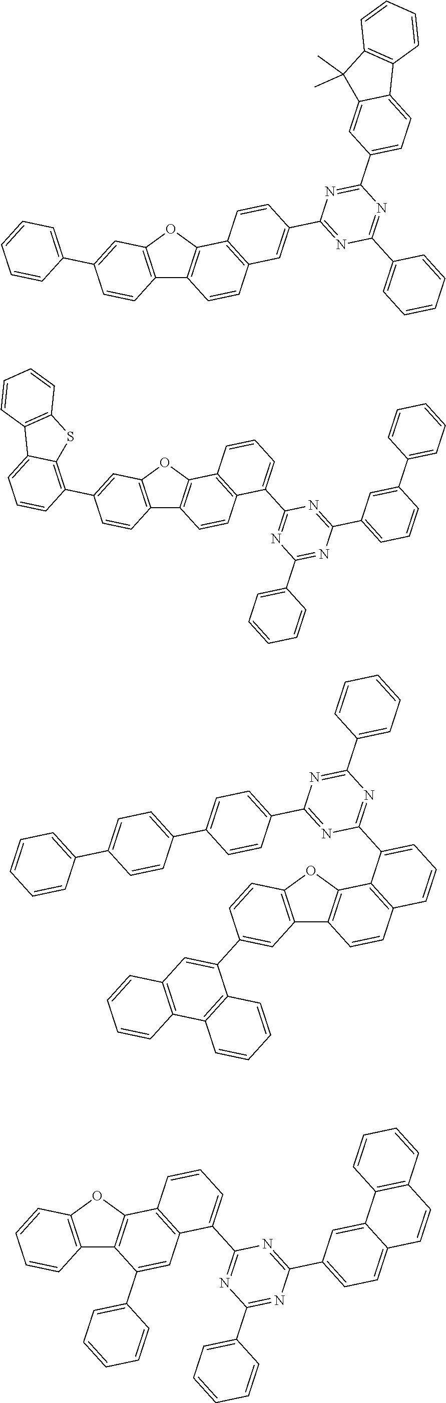 [SMILES: CC1(C)c2ccccc2-c2ccc(-c3nc(-c4ccccc4)nc(-c4ccc5c(ccc6c7ccc(-c8ccccc8)cc7oc56)c4)n3)cc21 ; c1ccc(-c2cccc(-c3nc(-c4ccccc4)nc(-c4cccc5c4ccc4c6ccc(-c7cccc8c7sc7ccccc78)cc6oc54)n3)c2)cc1 ; c1ccc(-c2ccc(-c3ccc(-c4nc(-c5ccccc5)nc(-c5cccc6ccc7c8cc(-c9cc%10ccccc%10c%10ccccc9%10)ccc8oc7c56)n4)cc3)cc2)cc1 ; c1ccc(-c2nc(-c3ccc4ccc5ccccc5c4c3)nc(-c3cccc4c3cc(-c3ccccc3)c3c5ccccc5oc43)n2)cc1]

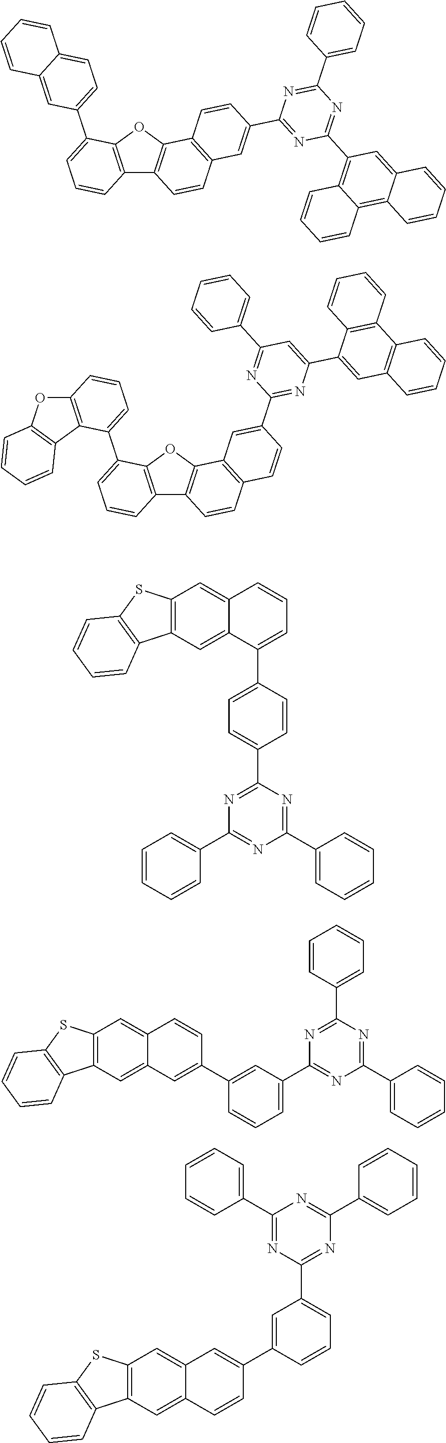

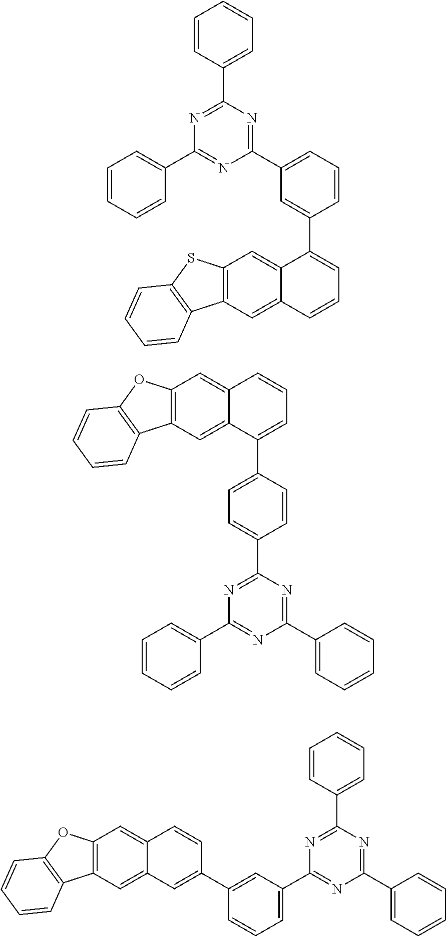

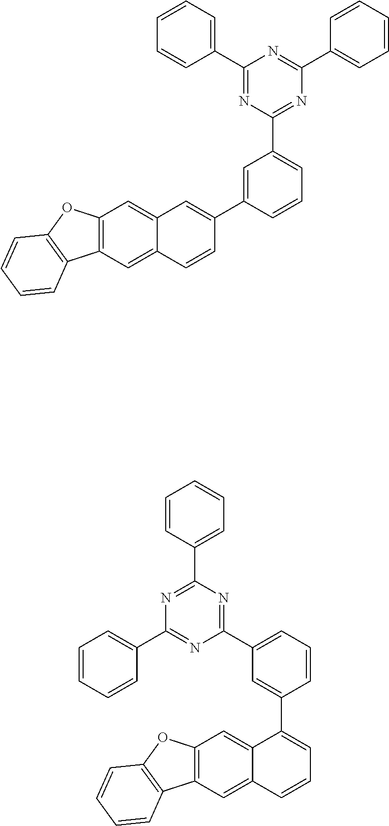 [SMILES: c1ccc(-c2nc(-c3ccccc3)nc(-c3cccc(-c4ccc5cc6c(cc5c4)oc4ccccc46)c3)n2)cc1 ; c1ccc(-c2nc(-c3ccccc3)nc(-c3cccc(-c4cccc5cc6c(cc45)oc4ccccc46)c3)n2)cc1]

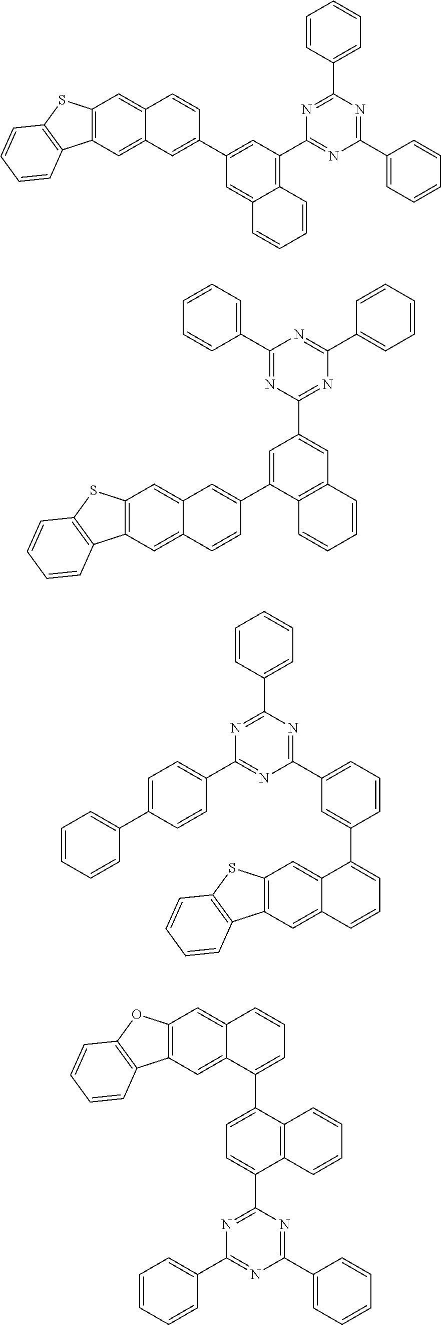 [SMILES: c1ccc(-c2nc(-c3ccccc3)nc(-c3cc(-c4ccc5cc6sc7ccccc7c6cc5c4)cc4ccccc34)n2)cc1 ; c1ccc(-c2nc(-c3ccccc3)nc(-c3cc(-c4ccc5cc6c(cc5c4)sc4ccccc46)c4ccccc4c3)n2)cc1 ; c1ccc(-c2ccc(-c3nc(-c4ccccc4)nc(-c4cccc(-c5cccc6cc7c(cc56)sc5ccccc57)c4)n3)cc2)cc1 ; c1ccc(-c2nc(-c3ccccc3)nc(-c3ccc(-c4cccc5cc6oc7ccccc7c6cc45)c4ccccc34)n2)cc1]

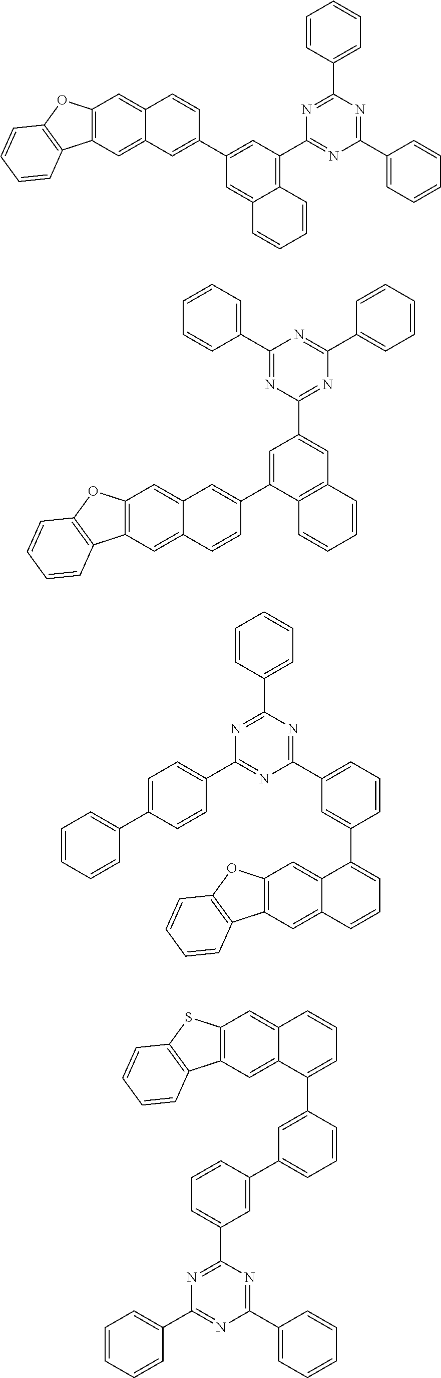 [SMILES: c1ccc(-c2nc(-c3ccccc3)nc(-c3cc(-c4ccc5cc6oc7ccccc7c6cc5c4)cc4ccccc34)n2)cc1 ; c1ccc(-c2nc(-c3ccccc3)nc(-c3cc(-c4ccc5cc6c(cc5c4)oc4ccccc46)c4ccccc4c3)n2)cc1 ; c1ccc(-c2ccc(-c3nc(-c4ccccc4)nc(-c4cccc(-c5cccc6cc7c(cc56)oc5ccccc57)c4)n3)cc2)cc1 ; c1ccc(-c2nc(-c3ccccc3)nc(-c3cccc(-c4cccc(-c5cccc6cc7sc8ccccc8c7cc56)c4)c3)n2)cc1]

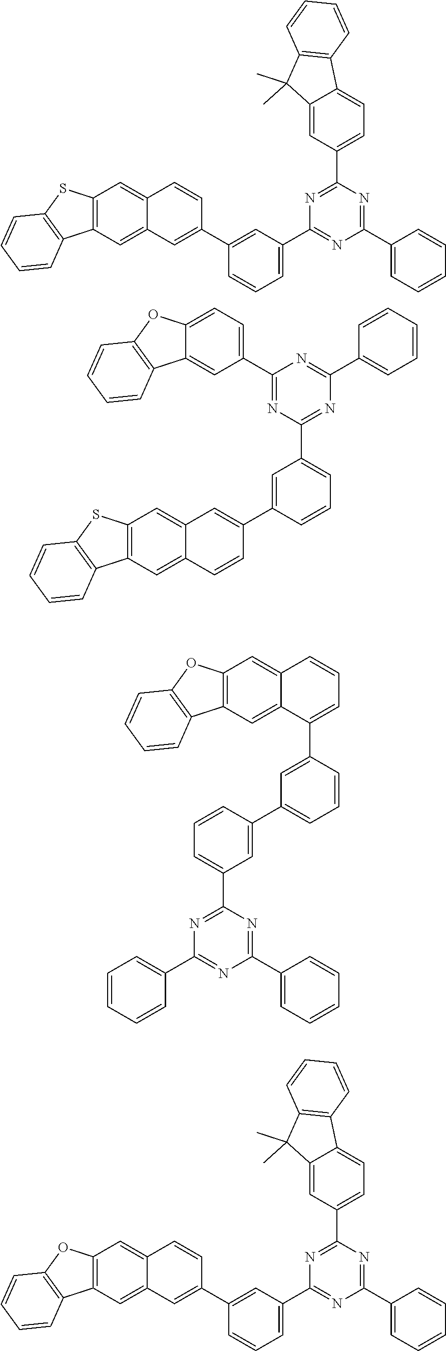

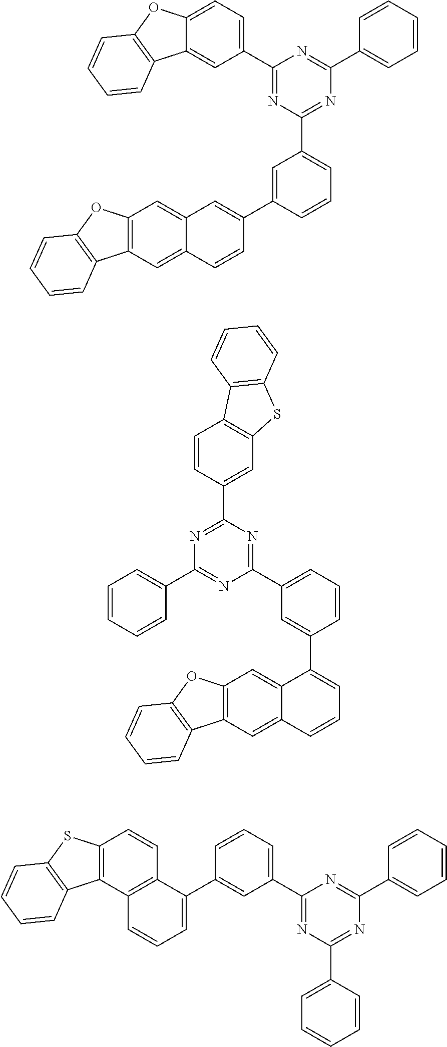 [SMILES: c1ccc(-c2nc(-c3cccc(-c4ccc5cc6c(cc5c4)oc4ccccc46)c3)nc(-c3ccc4oc5ccccc5c4c3)n2)cc1 ; c1ccc(-c2nc(-c3cccc(-c4cccc5cc6c(cc45)oc4ccccc46)c3)nc(-c3ccc4c(c3)sc3ccccc34)n2)cc1 ; c1ccc(-c2nc(-c3ccccc3)nc(-c3cccc(-c4cccc5c4ccc4sc6ccccc6c45)c3)n2)cc1]

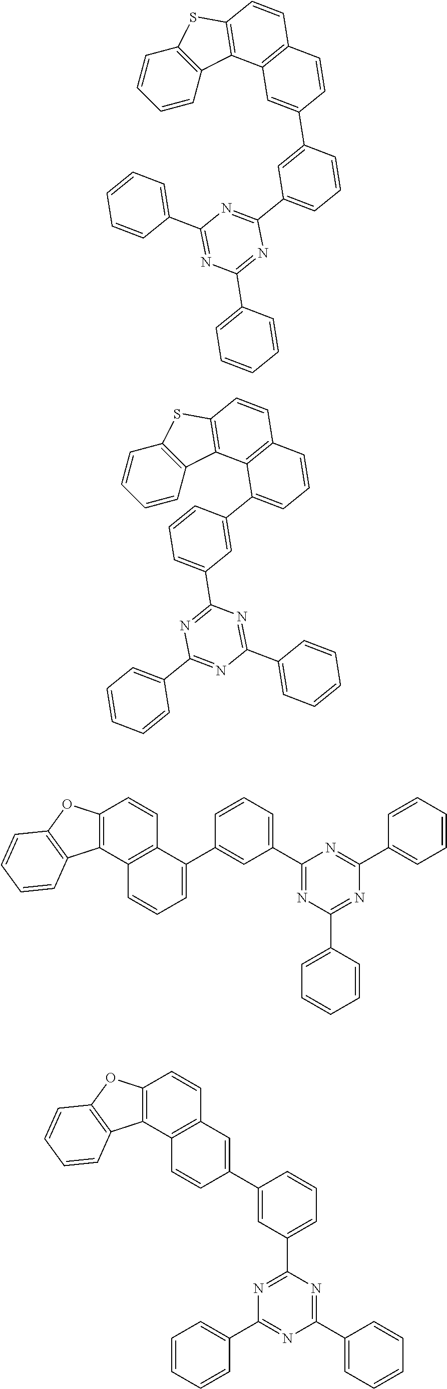

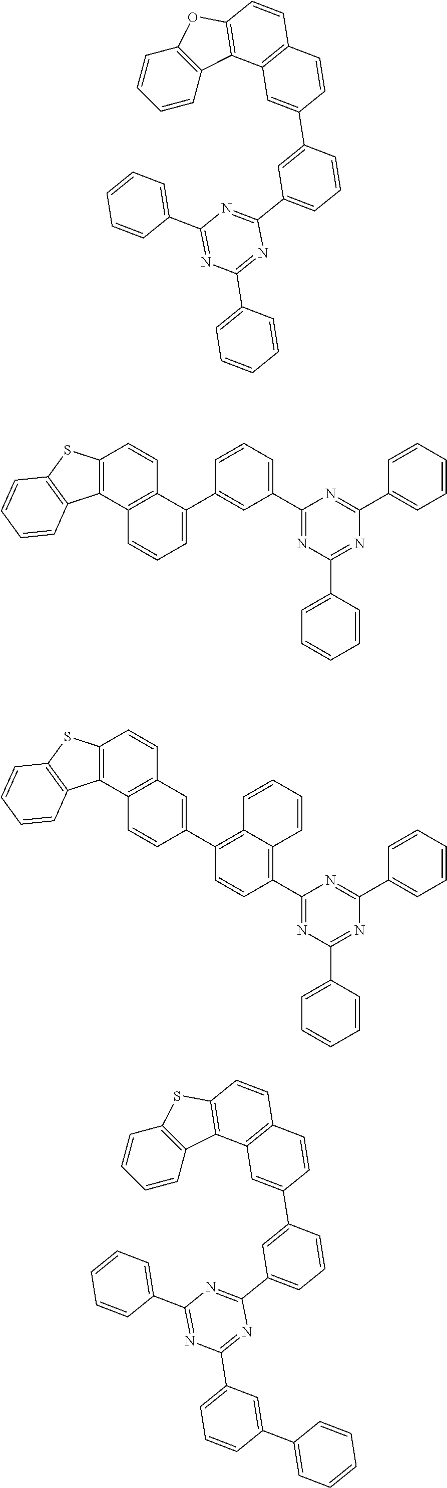 [SMILES: c1ccc(-c2nc(-c3ccccc3)nc(-c3cccc(-c4ccc5ccc6oc7ccccc7c6c5c4)c3)n2)cc1 ; c1ccc(-c2nc(-c3ccccc3)nc(-c3cccc(-c4cccc5c4ccc4sc6ccccc6c45)c3)n2)cc1 ; c1ccc(-c2nc(-c3ccccc3)nc(-c3ccc(-c4ccc5c(ccc6sc7ccccc7c65)c4)c4ccccc34)n2)cc1 ; c1ccc(-c2cccc(-c3nc(-c4ccccc4)nc(-c4cccc(-c5ccc6ccc7sc8ccccc8c7c6c5)c4)n3)c2)cc1]

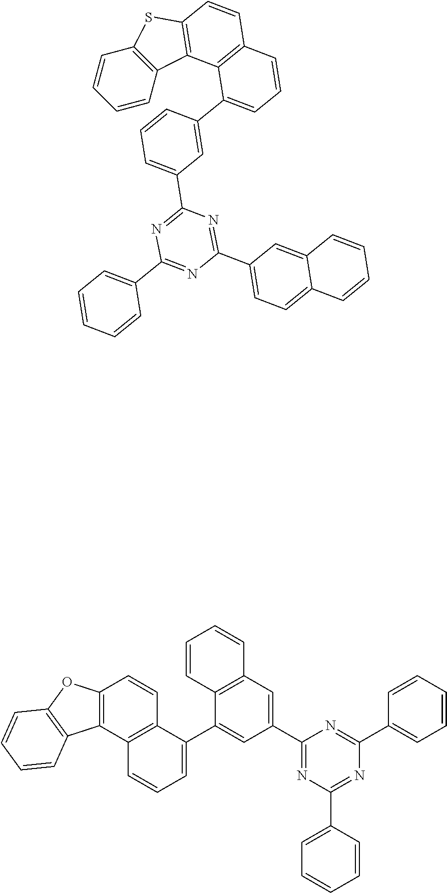 [SMILES: c1ccc(-c2nc(-c3cccc(-c4cccc5ccc6sc7ccccc7c6c45)c3)nc(-c3ccc4ccccc4c3)n2)cc1 ; c1ccc(-c2nc(-c3ccccc3)nc(-c3cc(-c4cccc5c4ccc4oc6ccccc6c45)c4ccccc4c3)n2)cc1]

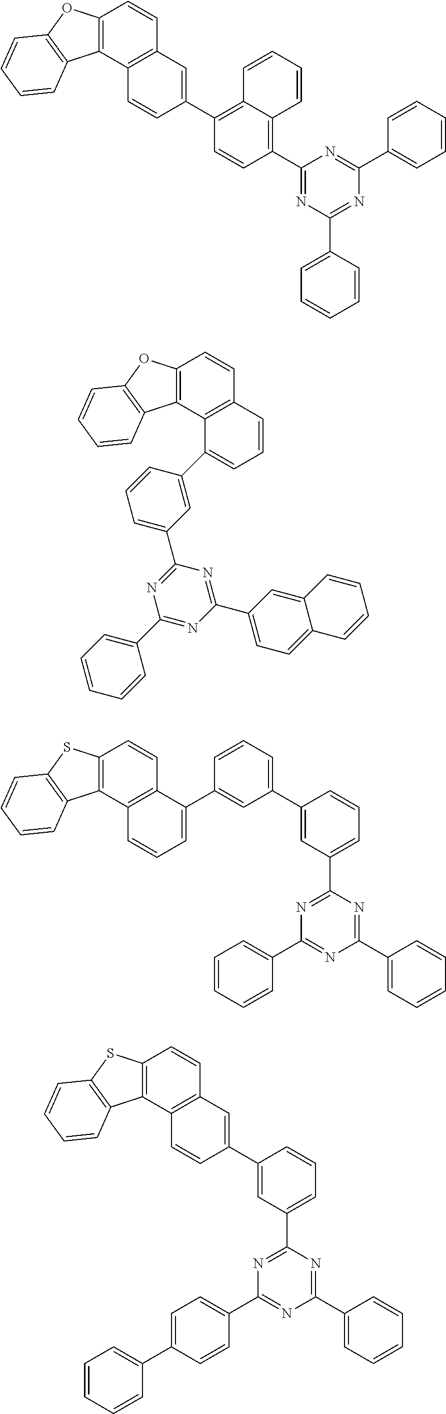

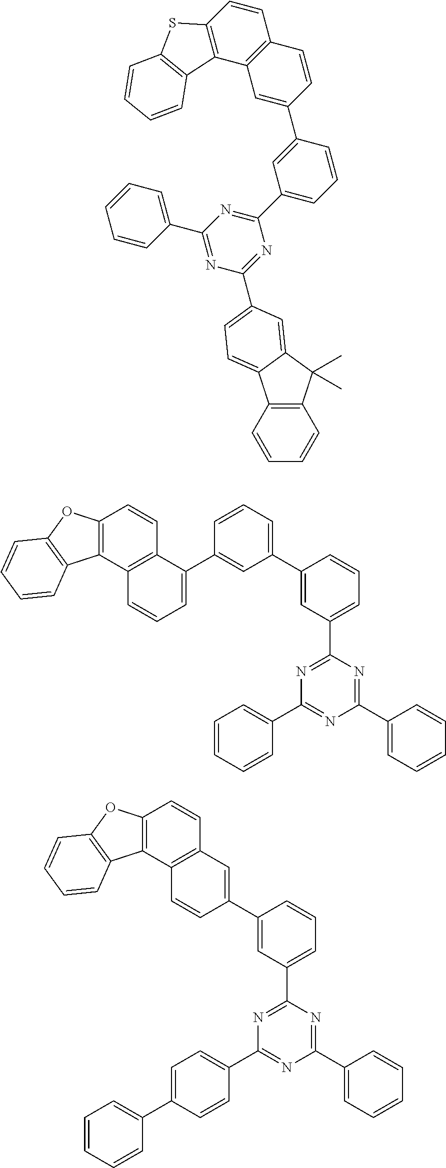 [SMILES: CC1(C)c2ccccc2-c2ccc(-c3nc(-c4ccccc4)nc(-c4cccc(-c5ccc6ccc7sc8ccccc8c7c6c5)c4)n3)cc21 ; c1ccc(-c2nc(-c3ccccc3)nc(-c3cccc(-c4cccc(-c5cccc6c5ccc5oc7ccccc7c56)c4)c3)n2)cc1 ; c1ccc(-c2ccc(-c3nc(-c4ccccc4)nc(-c4cccc(-c5ccc6c(ccc7oc8ccccc8c76)c5)c4)n3)cc2)cc1]

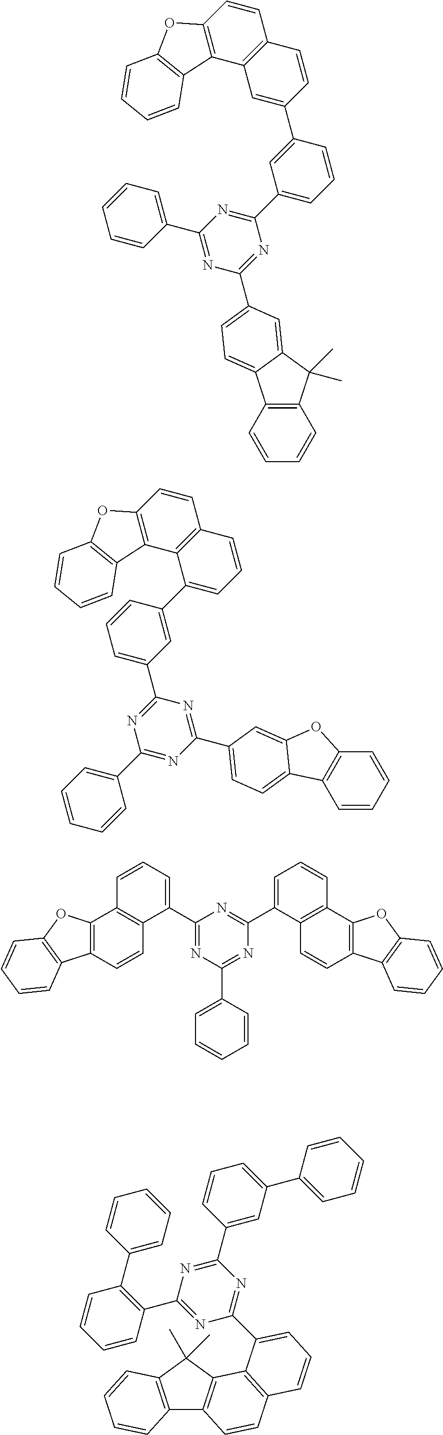 [SMILES: CC1(C)c2ccccc2-c2ccc(-c3nc(-c4ccccc4)nc(-c4cccc(-c5ccc6ccc7oc8ccccc8c7c6c5)c4)n3)cc21 ; c1ccc(-c2nc(-c3cccc(-c4cccc5ccc6oc7ccccc7c6c45)c3)nc(-c3ccc4c(c3)oc3ccccc34)n2)cc1 ; c1ccc(-c2nc(-c3cccc4c3ccc3c5ccccc5oc43)nc(-c3cccc4c3ccc3c5ccccc5oc43)n2)cc1 ; CC1(C)c2ccccc2-c2ccc3cccc(-c4nc(-c5cccc(-c6ccccc6)c5)nc(-c5ccccc5-c5ccccc5)n4)c3c21]

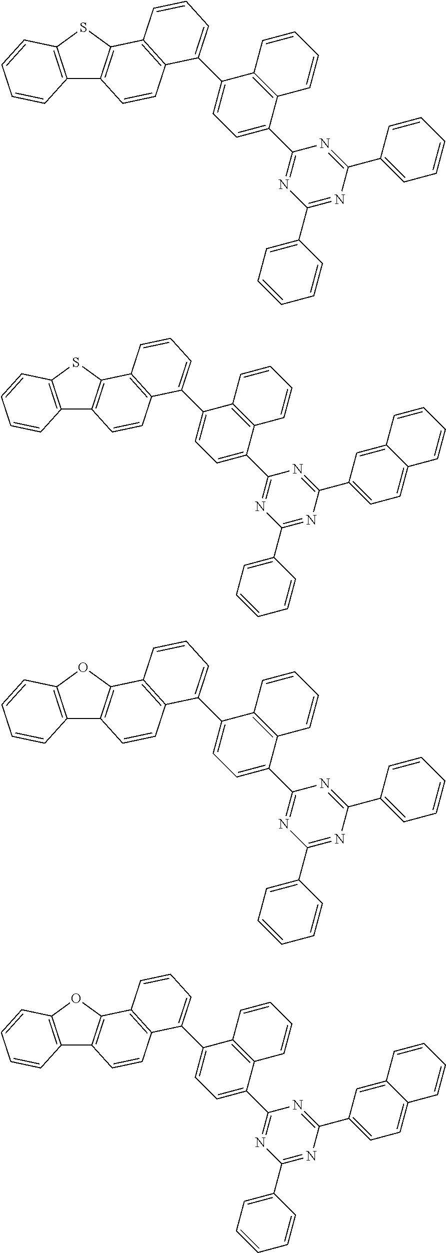 [SMILES: c1ccc(-c2nc(-c3ccccc3)nc(-c3ccc(-c4cccc5c4ccc4c6ccccc6sc54)c4ccccc34)n2)cc1 ; c1ccc(-c2nc(-c3ccc4ccccc4c3)nc(-c3ccc(-c4cccc5c4ccc4c6ccccc6sc54)c4ccccc34)n2)cc1 ; c1ccc(-c2nc(-c3ccccc3)nc(-c3ccc(-c4cccc5c4ccc4c6ccccc6oc54)c4ccccc34)n2)cc1 ; c1ccc(-c2nc(-c3ccc4ccccc4c3)nc(-c3ccc(-c4cccc5c4ccc4c6ccccc6oc54)c4ccccc34)n2)cc1]

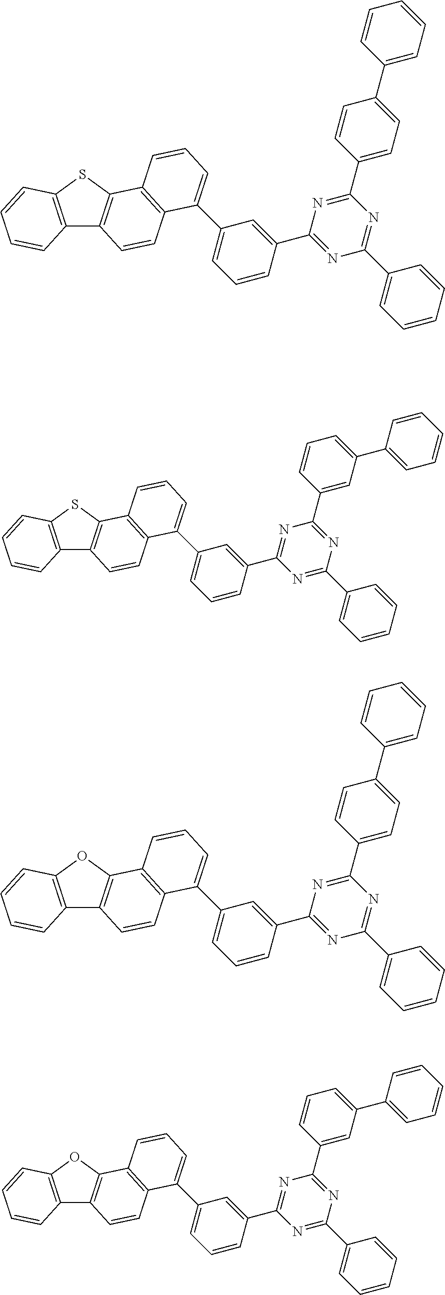 [SMILES: c1ccc(-c2ccc(-c3nc(-c4ccccc4)nc(-c4cccc(-c5cccc6c5ccc5c7ccccc7sc65)c4)n3)cc2)cc1 ; c1ccc(-c2cccc(-c3nc(-c4ccccc4)nc(-c4cccc(-c5cccc6c5ccc5c7ccccc7sc65)c4)n3)c2)cc1 ; c1ccc(-c2ccc(-c3nc(-c4ccccc4)nc(-c4cccc(-c5cccc6c5ccc5c7ccccc7oc65)c4)n3)cc2)cc1 ; c1ccc(-c2cccc(-c3nc(-c4ccccc4)nc(-c4cccc(-c5cccc6c5ccc5c7ccccc7oc65)c4)n3)c2)cc1]

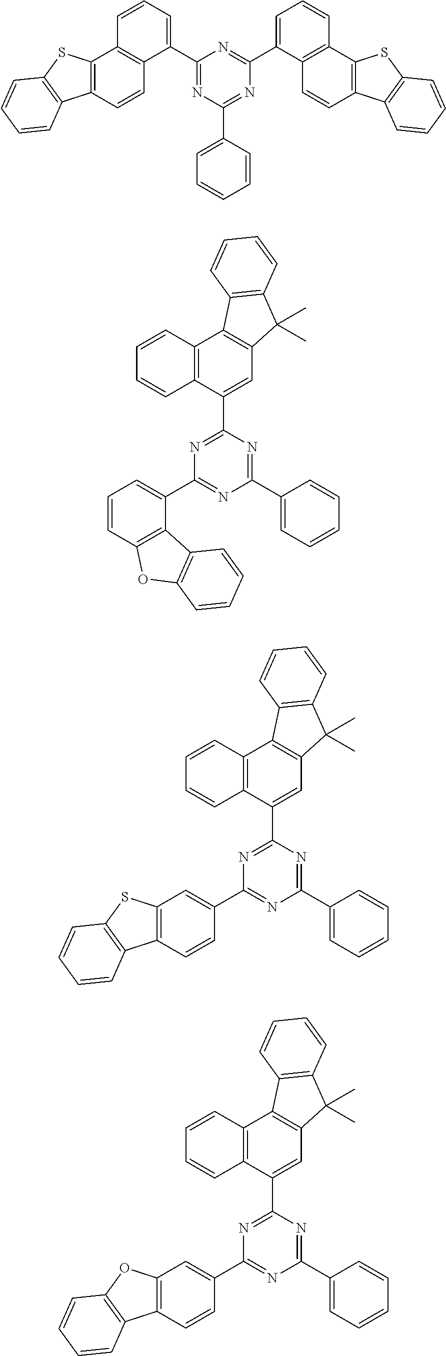

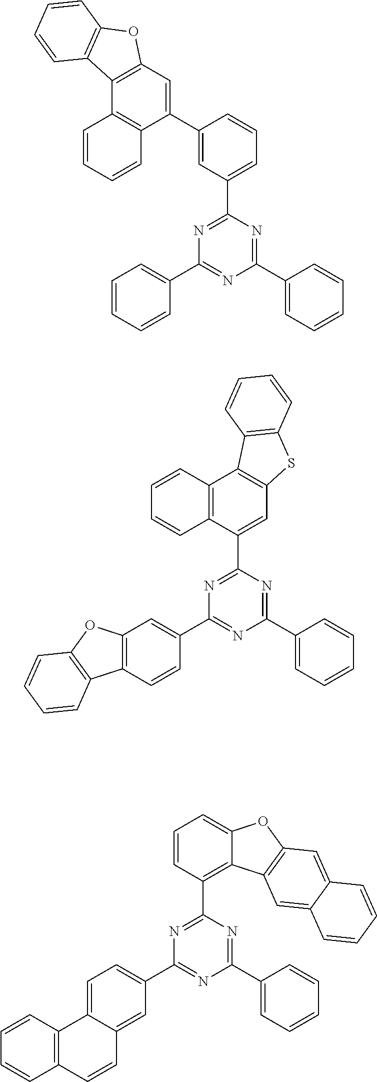 [SMILES: c1ccc(-c2nc(-c3ccccc3)nc(-c3cccc(-c4cc5oc6ccccc6c5c5ccccc45)c3)n2)cc1 ; c1ccc(-c2nc(-c3ccc4c(c3)oc3ccccc34)nc(-c3cc4sc5ccccc5c4c4ccccc34)n2)cc1 ; c1ccc(-c2nc(-c3ccc4c(ccc5ccccc54)c3)nc(-c3cccc4oc5cc6ccccc6cc5c34)n2)cc1]

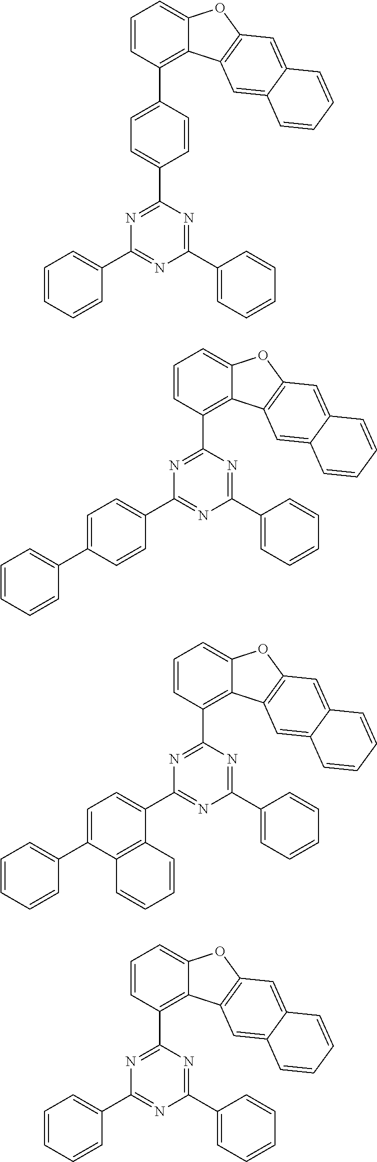

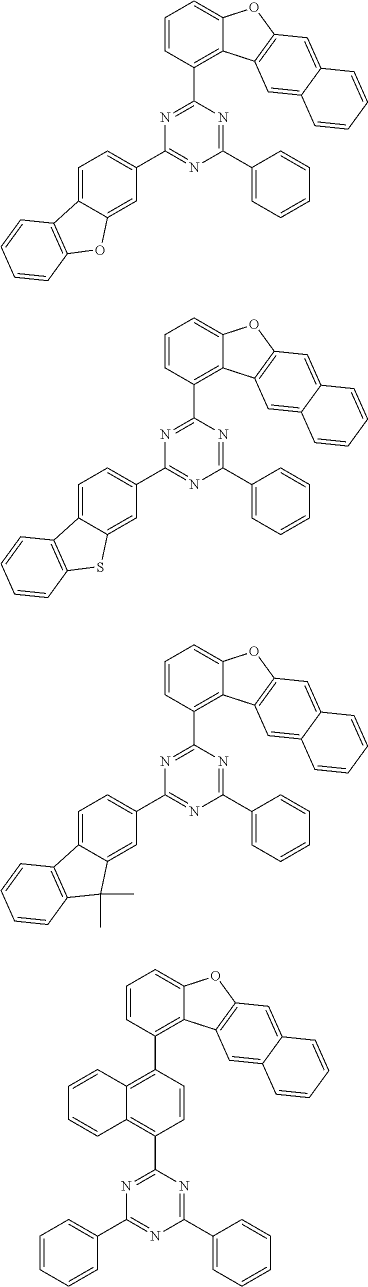 [SMILES: c1ccc(-c2nc(-c3ccc4c(c3)oc3ccccc34)nc(-c3cccc4oc5cc6ccccc6cc5c34)n2)cc1 ; c1ccc(-c2nc(-c3ccc4c(c3)sc3ccccc34)nc(-c3cccc4oc5cc6ccccc6cc5c34)n2)cc1 ; CC1(C)c2ccccc2-c2ccc(-c3nc(-c4ccccc4)nc(-c4cccc5oc6cc7ccccc7cc6c45)n3)cc21 ; c1ccc(-c2nc(-c3ccccc3)nc(-c3ccc(-c4cccc5oc6cc7ccccc7cc6c45)c4ccccc34)n2)cc1]

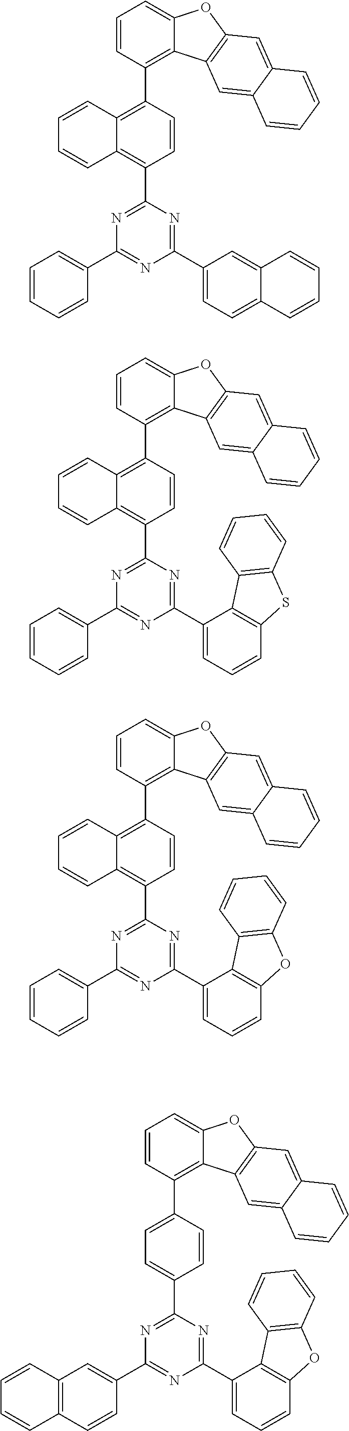 [SMILES: c1ccc(-c2nc(-c3ccc4ccccc4c3)nc(-c3ccc(-c4cccc5oc6cc7ccccc7cc6c45)c4ccccc34)n2)cc1 ; c1ccc(-c2nc(-c3ccc(-c4cccc5oc6cc7ccccc7cc6c45)c4ccccc34)nc(-c3cccc4sc5ccccc5c34)n2)cc1 ; c1ccc(-c2nc(-c3ccc(-c4cccc5oc6cc7ccccc7cc6c45)c4ccccc34)nc(-c3cccc4oc5ccccc5c34)n2)cc1 ; c1ccc2cc(-c3nc(-c4ccc(-c5cccc6oc7cc8ccccc8cc7c56)cc4)nc(-c4cccc5oc6ccccc6c45)n3)ccc2c1]

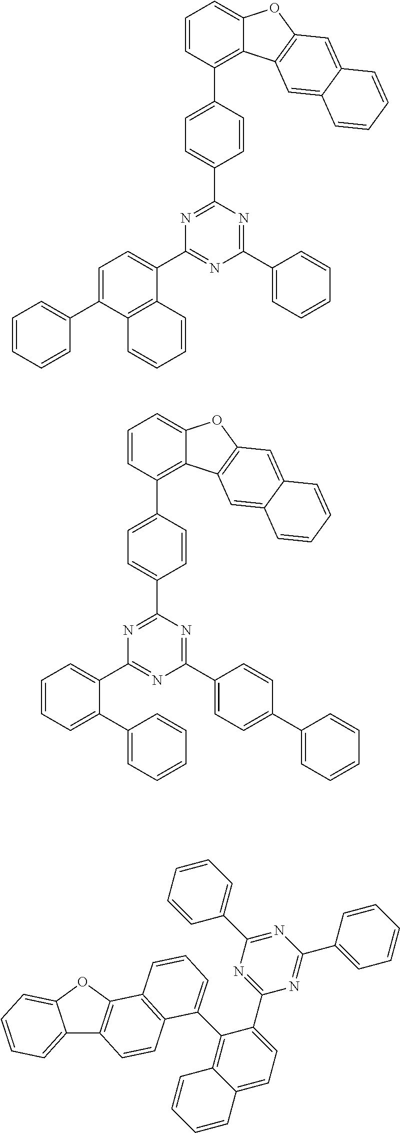

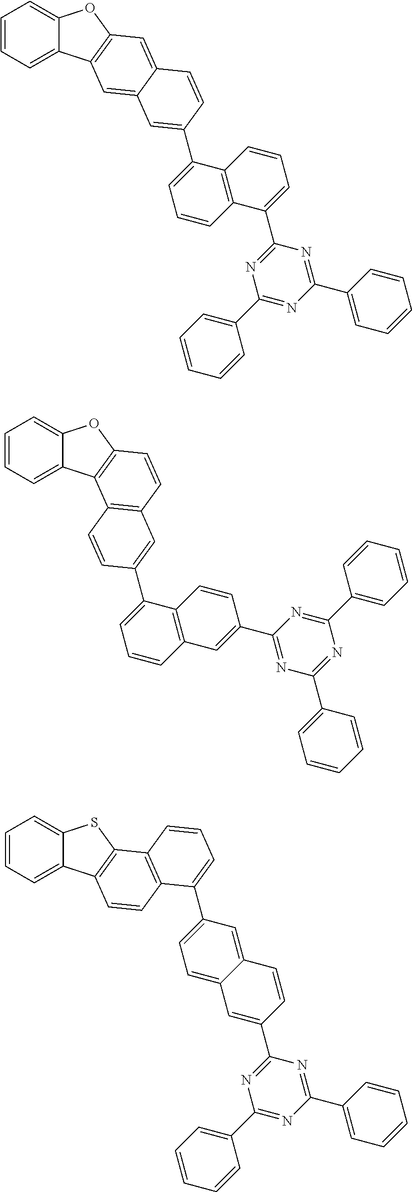 [SMILES: c1ccc(-c2nc(-c3ccccc3)nc(-c3cccc4c(-c5ccc6cc7oc8ccccc8c7cc6c5)cccc34)n2)cc1 ; c1ccc(-c2nc(-c3ccccc3)nc(-c3ccc4c(-c5ccc6c(ccc7oc8ccccc8c76)c5)cccc4c3)n2)cc1 ; c1ccc(-c2nc(-c3ccccc3)nc(-c3ccc4cc(-c5cccc6c5ccc5c7ccccc7sc65)ccc4c3)n2)cc1]

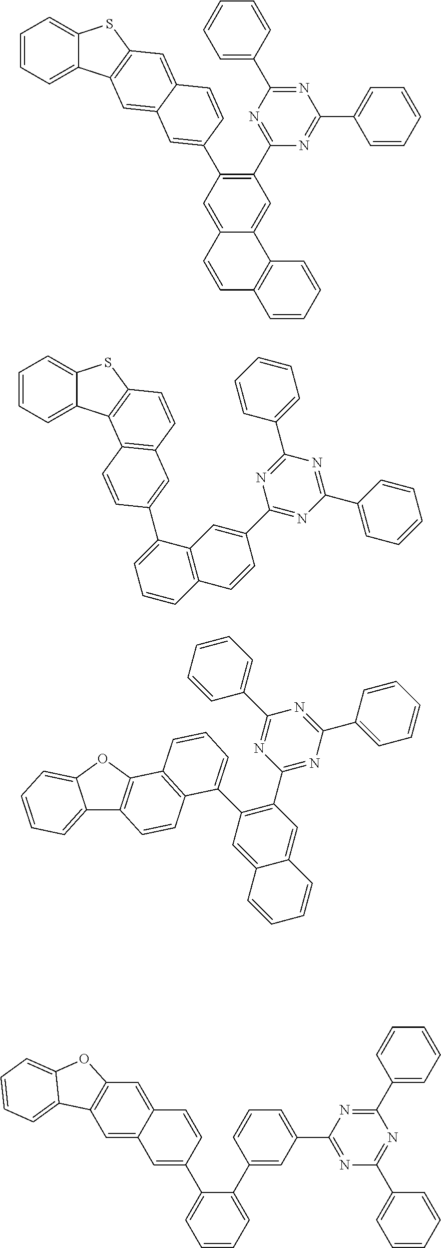

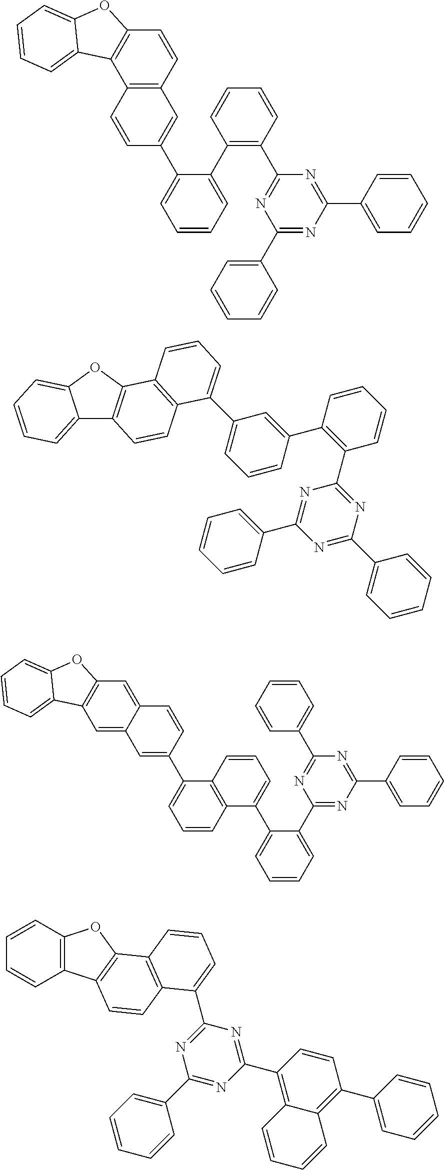

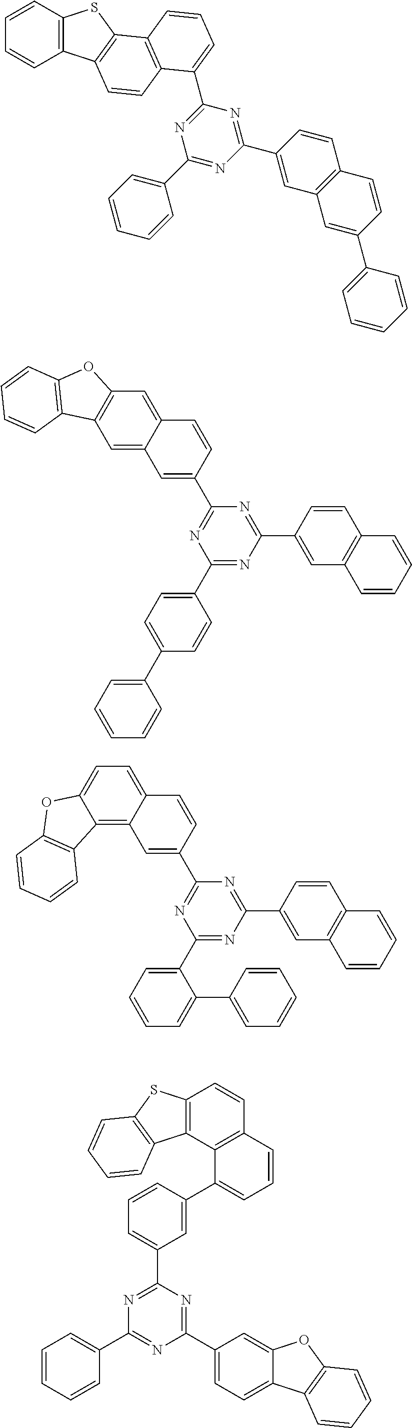

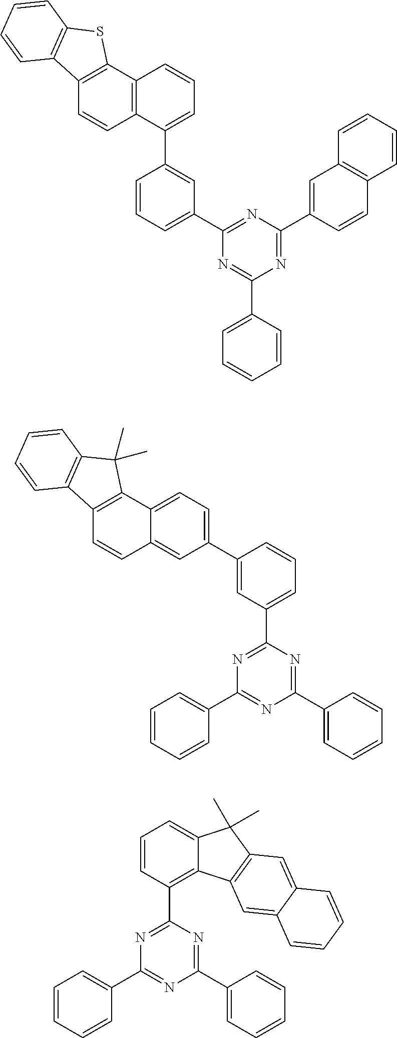 [SMILES: c1ccc(-c2nc(-c3cccc(-c4cccc5c4ccc4c6ccccc6sc54)c3)nc(-c3ccc4ccccc4c3)n2)cc1 ; CC1(C)c2ccccc2-c2ccc3cc(-c4cccc(-c5nc(-c6ccccc6)nc(-c6ccccc6)n5)c4)ccc3c21 ; CC1(C)c2cc3ccccc3cc2-c2c(-c3nc(-c4ccccc4)nc(-c4ccccc4)n3)cccc21]

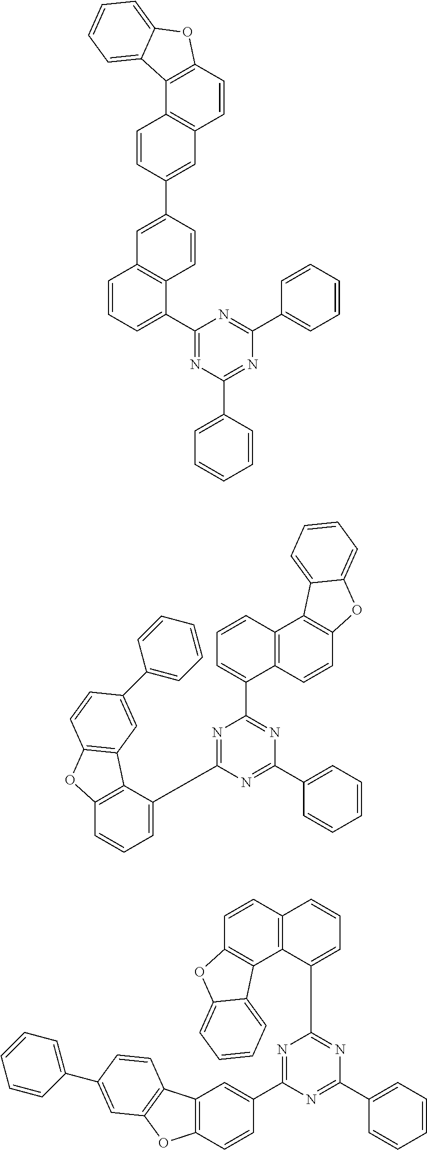 [SMILES: c1ccc(-c2nc(-c3ccccc3)nc(-c3cccc4cc(-c5ccc6c(ccc7oc8ccccc8c76)c5)ccc34)n2)cc1 ; c1ccc(-c2ccc3oc4cccc(-c5nc(-c6ccccc6)nc(-c6cccc7c6ccc6oc8ccccc8c67)n5)c4c3c2)cc1 ; c1ccc(-c2ccc3c(c2)oc2ccc(-c4nc(-c5ccccc5)nc(-c5cccc6ccc7oc8ccccc8c7c56)n4)cc23)cc1]

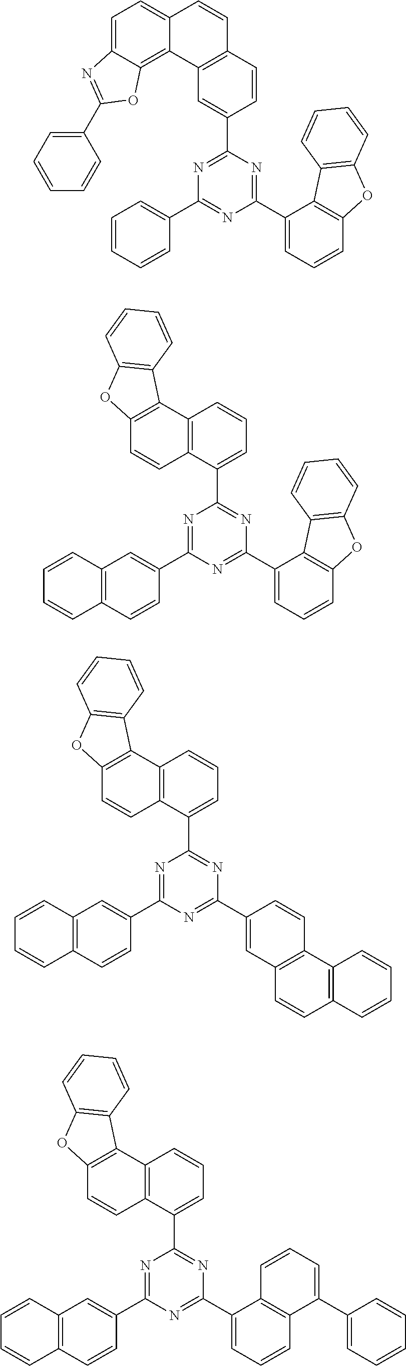

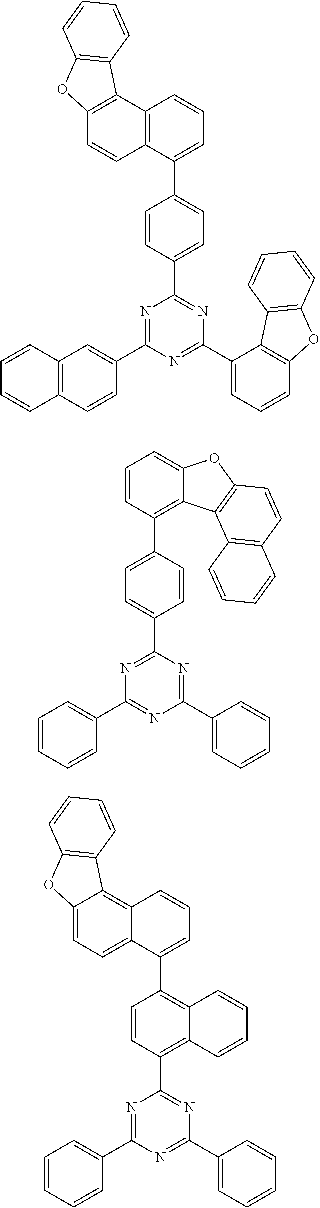

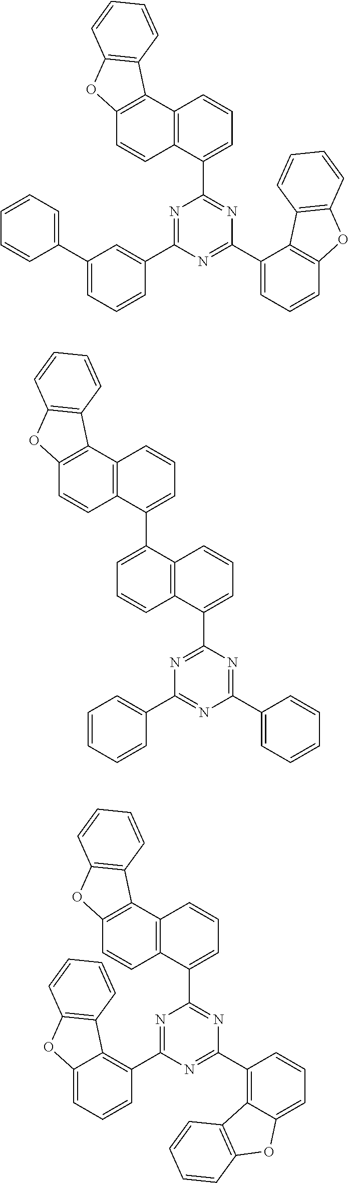 [SMILES: c1ccc(-c2cccc(-c3nc(-c4cccc5c4ccc4oc6ccccc6c45)nc(-c4cccc5oc6ccccc6c45)n3)c2)cc1 ; c1ccc(-c2nc(-c3ccccc3)nc(-c3cccc4c(-c5cccc6c5ccc5oc7ccccc7c56)cccc34)n2)cc1 ; c1ccc2c(c1)oc1cccc(-c3nc(-c4cccc5c4ccc4oc6ccccc6c45)nc(-c4cccc5oc6ccccc6c45)n3)c12]

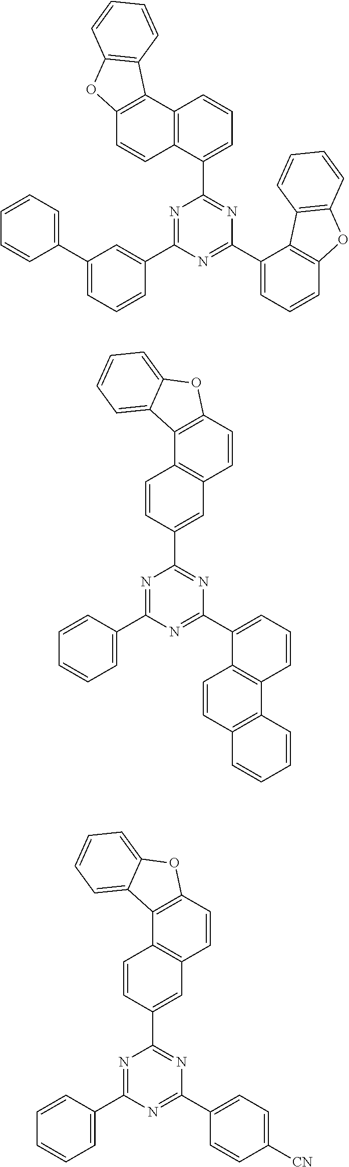

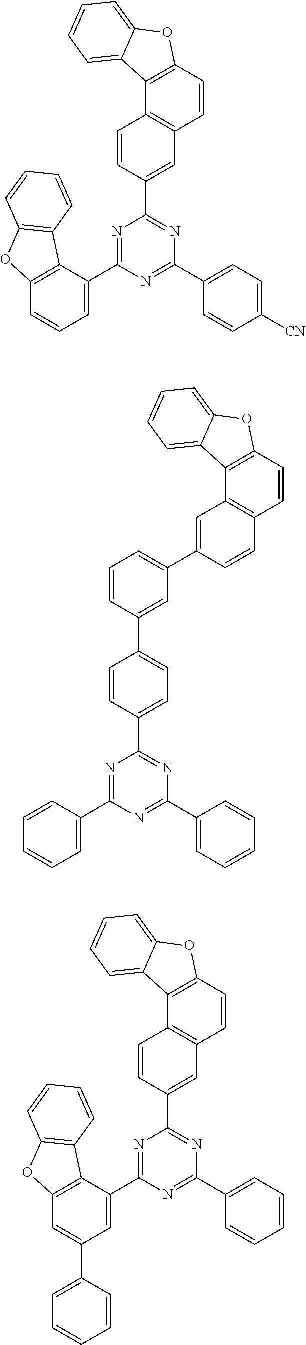 [SMILES: N#Cc1ccc(-c2nc(-c3ccc4c(ccc5oc6ccccc6c54)c3)nc(-c3cccc4oc5ccccc5c34)n2)cc1 ; c1ccc(-c2nc(-c3ccccc3)nc(-c3ccc(-c4cccc(-c5ccc6ccc7oc8ccccc8c7c6c5)c4)cc3)n2)cc1 ; c1ccc(-c2cc(-c3nc(-c4ccccc4)nc(-c4ccc5c(ccc6oc7ccccc7c65)c4)n3)c3c(c2)oc2ccccc23)cc1]

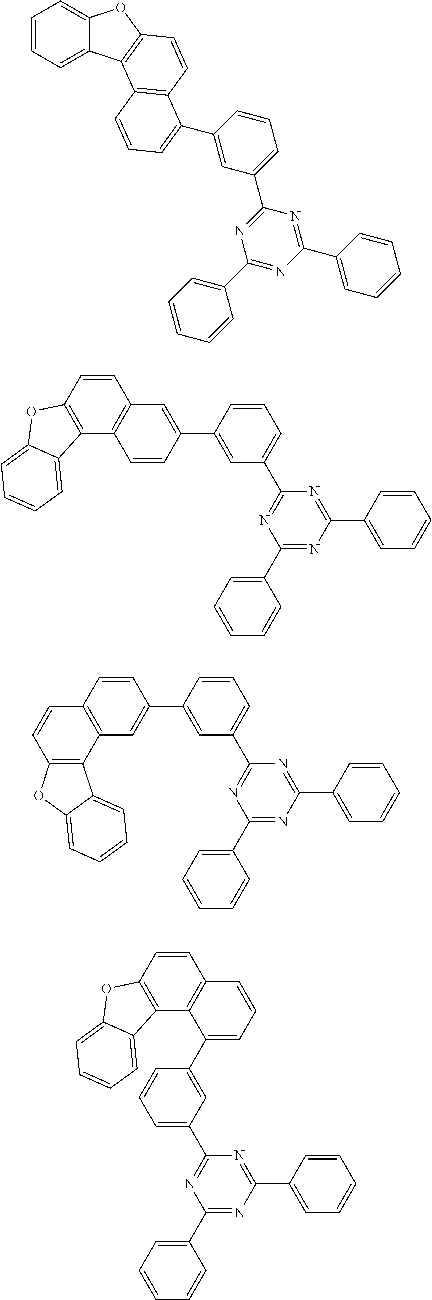

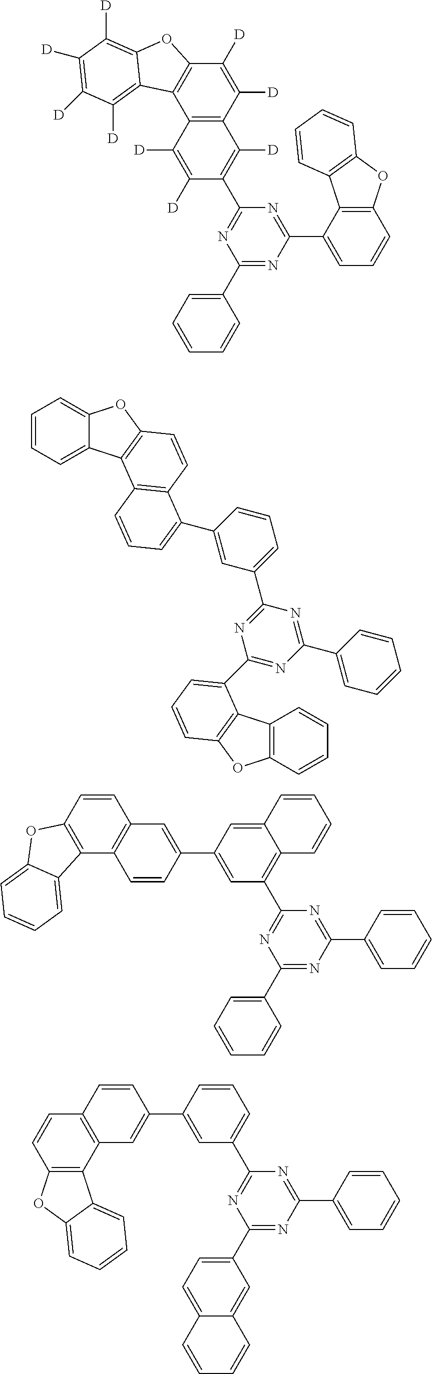

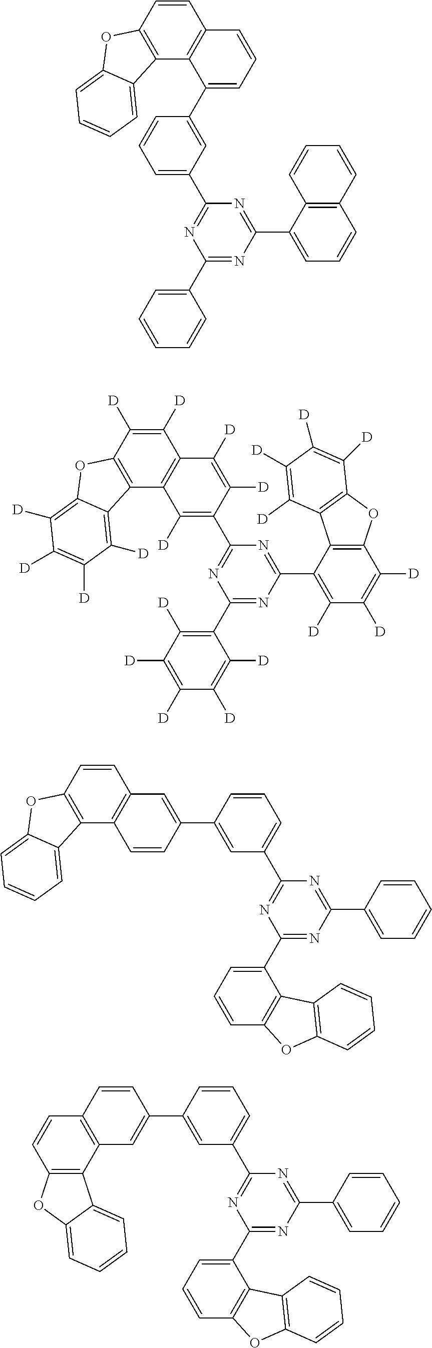 [SMILES: c1ccc(-c2nc(-c3cccc(-c4cccc5ccc6oc7ccccc7c6c45)c3)nc(-c3cccc4ccccc34)n2)cc1 ; [2H]c1c([2H])c([2H])c(-c2nc(-c3c([2H])c([2H])c4c([2H])c([2H])c5oc6c([2H])c([2H])c([2H])c([2H])c6c5c4c3[2H])nc(-c3c([2H])c([2H])c([2H])c4oc5c([2H])c([2H])c([2H])c([2H])c5c34)n2)c([2H])c1[2H] ; c1ccc(-c2nc(-c3cccc(-c4ccc5c(ccc6oc7ccccc7c65)c4)c3)nc(-c3cccc4oc5ccccc5c34)n2)cc1 ; c1ccc(-c2nc(-c3cccc(-c4ccc5ccc6oc7ccccc7c6c5c4)c3)nc(-c3cccc4oc5ccccc5c34)n2)cc1]

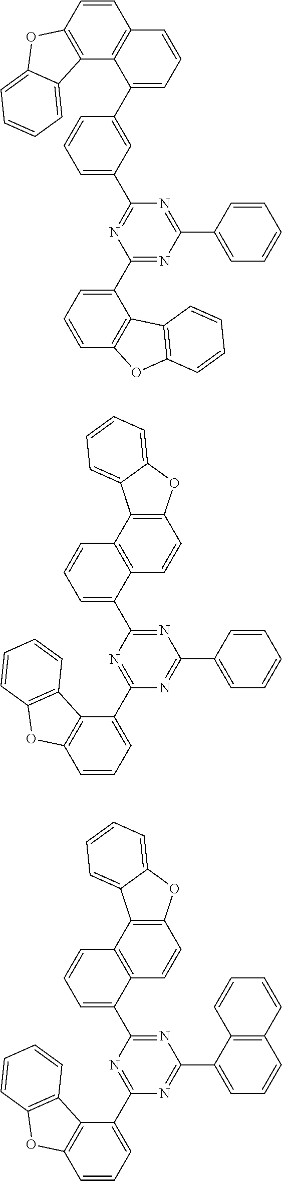 [SMILES: c1ccc(-c2nc(-c3cccc(-c4cccc5ccc6oc7ccccc7c6c45)c3)nc(-c3cccc4oc5ccccc5c34)n2)cc1 ; c1ccc(-c2nc(-c3cccc4c3ccc3oc5ccccc5c34)nc(-c3cccc4oc5ccccc5c34)n2)cc1 ; c1ccc2c(-c3nc(-c4cccc5c4ccc4oc6ccccc6c45)nc(-c4cccc5oc6ccccc6c45)n3)cccc2c1]

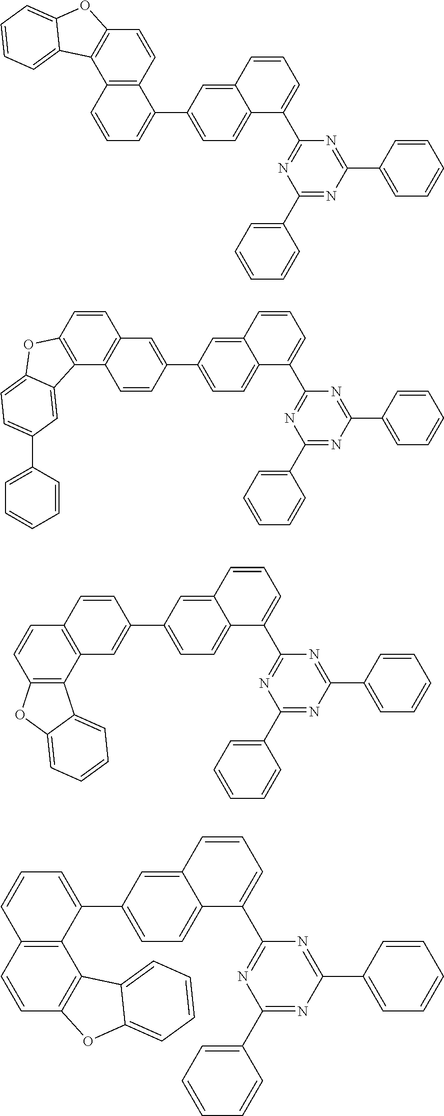

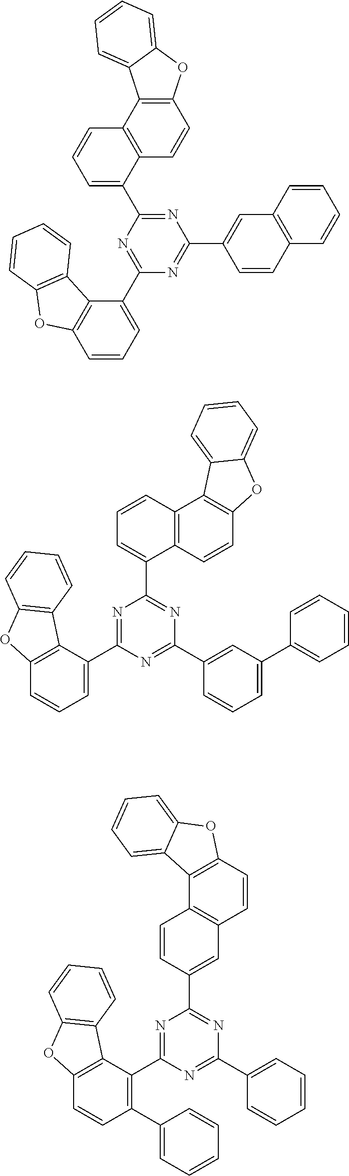 [SMILES: c1ccc2cc(-c3nc(-c4cccc5c4ccc4oc6ccccc6c45)nc(-c4cccc5oc6ccccc6c45)n3)ccc2c1 ; c1ccc(-c2cccc(-c3nc(-c4cccc5c4ccc4oc6ccccc6c45)nc(-c4cccc5oc6ccccc6c45)n3)c2)cc1 ; c1ccc(-c2nc(-c3ccc4c(ccc5oc6ccccc6c54)c3)nc(-c3c(-c4ccccc4)ccc4oc5ccccc5c34)n2)cc1]

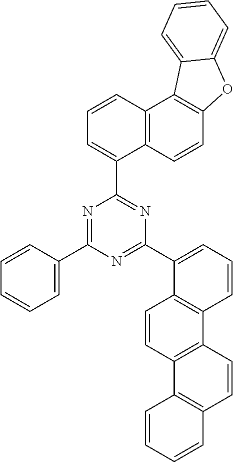 [SMILES: c1ccc(-c2nc(-c3cccc4c3ccc3c5ccccc5ccc43)nc(-c3cccc4c3ccc3oc5ccccc5c34)n2)cc1]

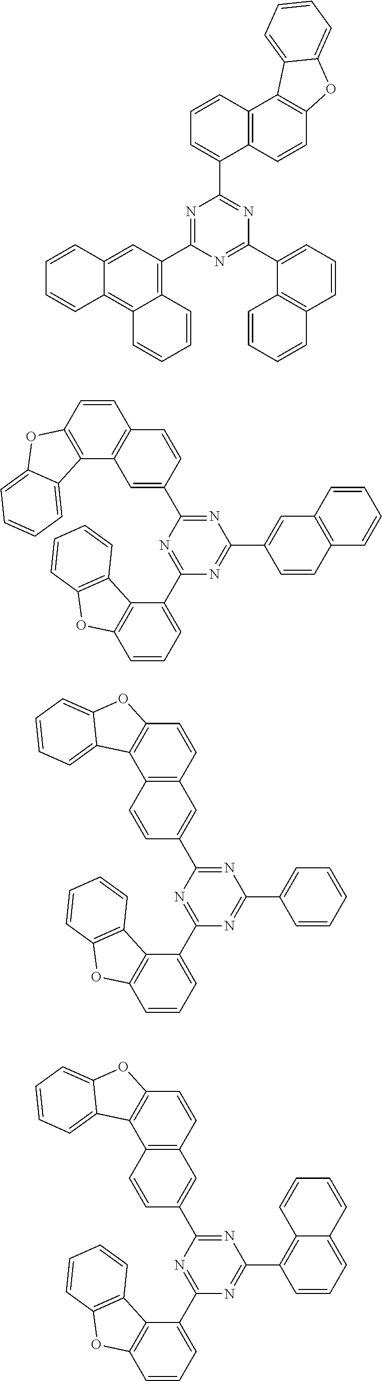

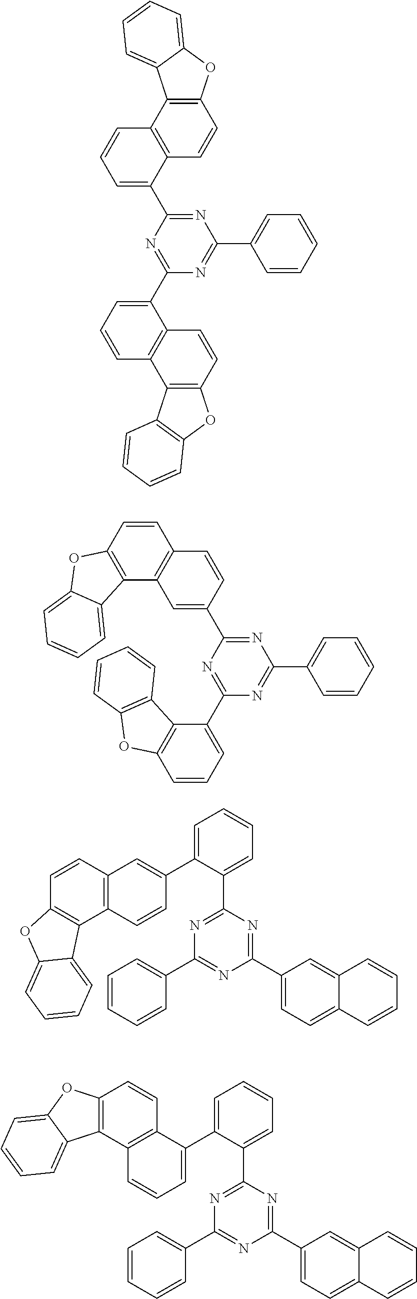

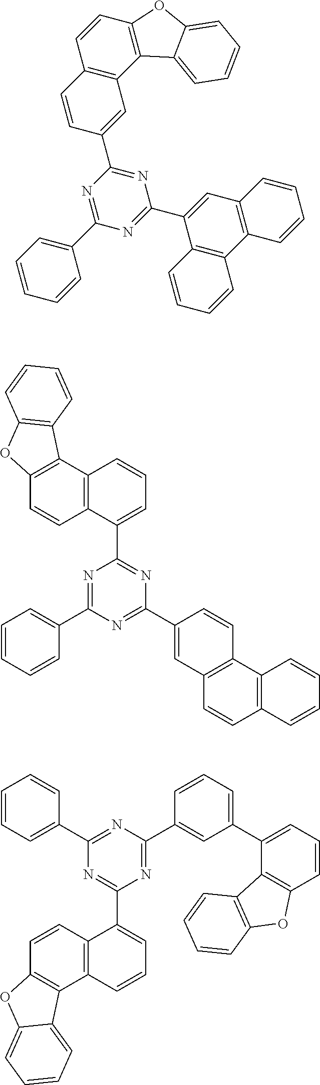 [SMILES: c1ccc(-c2nc(-c3ccc4ccc5oc6ccccc6c5c4c3)nc(-c3cc4ccccc4c4ccccc34)n2)cc1 ; c1ccc(-c2nc(-c3ccc4c(ccc5ccccc54)c3)nc(-c3cccc4c3ccc3oc5ccccc5c34)n2)cc1 ; c1ccc(-c2nc(-c3cccc(-c4cccc5oc6ccccc6c45)c3)nc(-c3cccc4c3ccc3oc5ccccc5c34)n2)cc1]

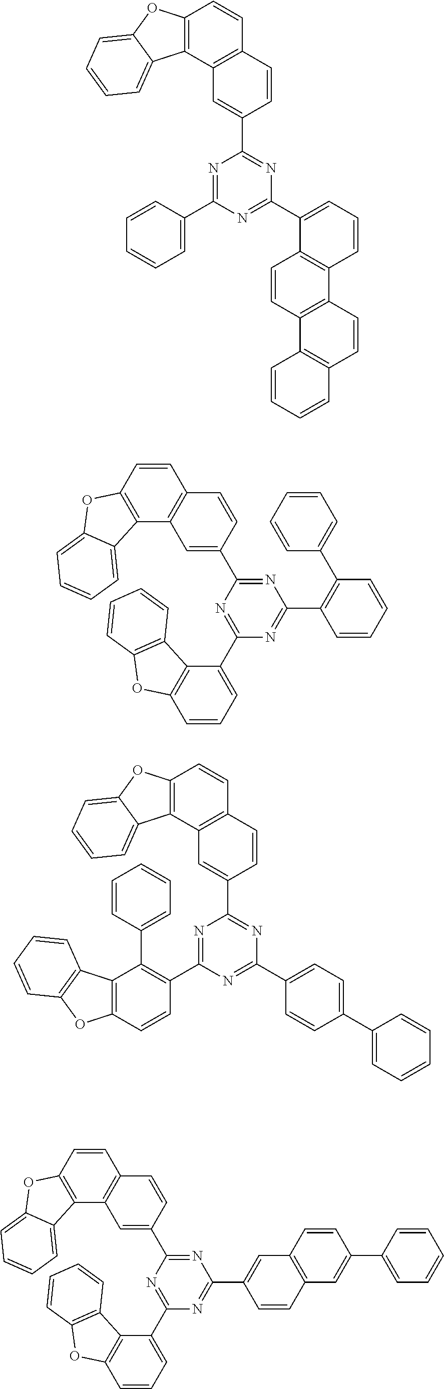

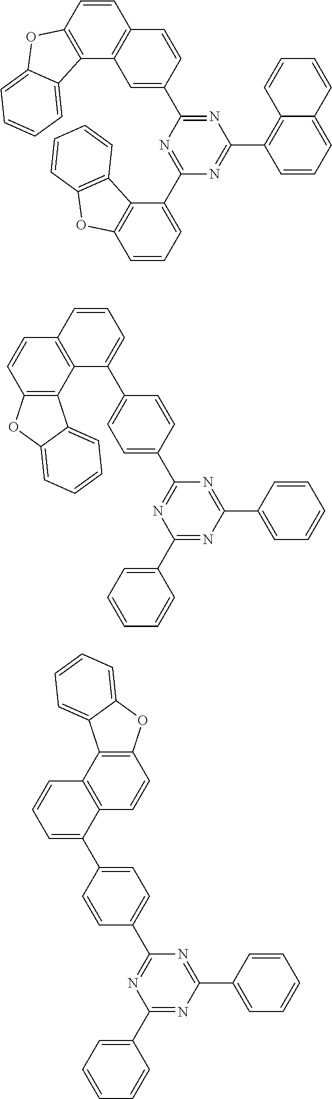

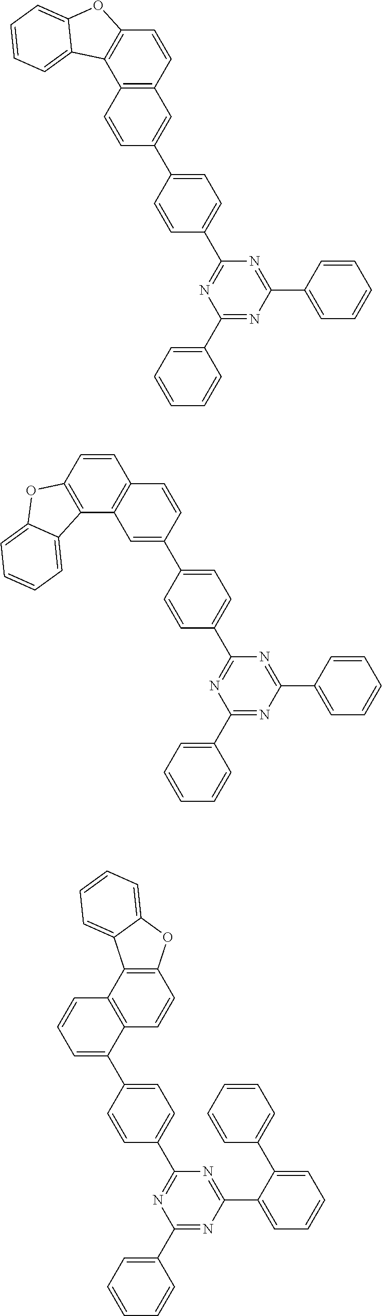 [SMILES: c1ccc(-c2nc(-c3ccccc3)nc(-c3ccc(-c4ccc5c(ccc6oc7ccccc7c65)c4)cc3)n2)cc1 ; c1ccc(-c2nc(-c3ccccc3)nc(-c3ccc(-c4ccc5ccc6oc7ccccc7c6c5c4)cc3)n2)cc1 ; c1ccc(-c2nc(-c3ccc(-c4cccc5c4ccc4oc6ccccc6c45)cc3)nc(-c3ccccc3-c3ccccc3)n2)cc1]

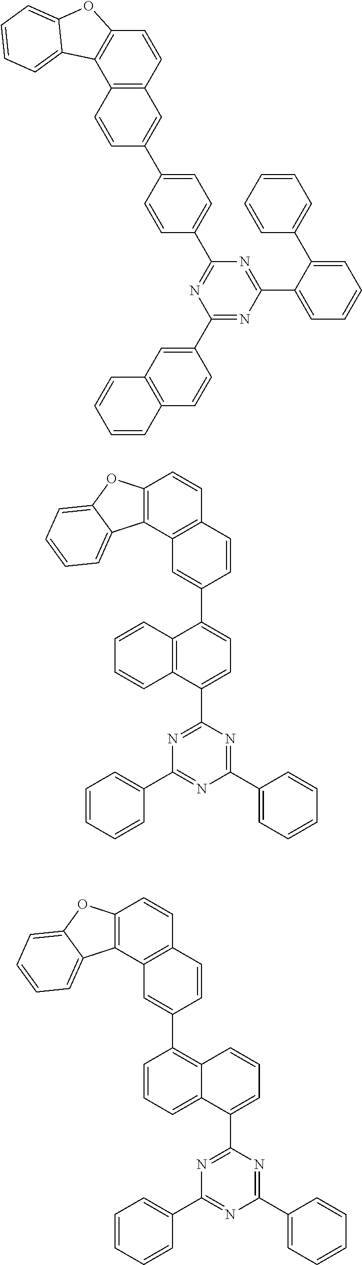

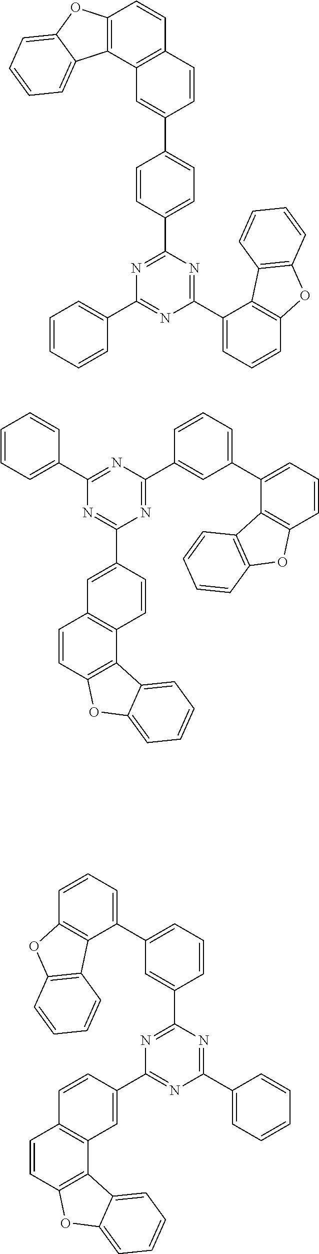 [SMILES: c1ccc(-c2nc(-c3ccc(-c4ccc5ccc6oc7ccccc7c6c5c4)cc3)nc(-c3cccc4oc5ccccc5c34)n2)cc1 ; c1ccc(-c2nc(-c3cccc(-c4cccc5oc6ccccc6c45)c3)nc(-c3ccc4c(ccc5oc6ccccc6c54)c3)n2)cc1 ; c1ccc(-c2nc(-c3cccc(-c4cccc5oc6ccccc6c45)c3)nc(-c3ccc4ccc5oc6ccccc6c5c4c3)n2)cc1]

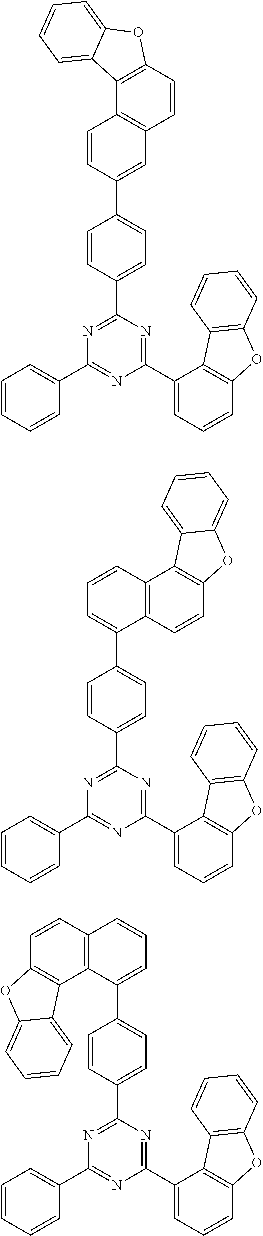

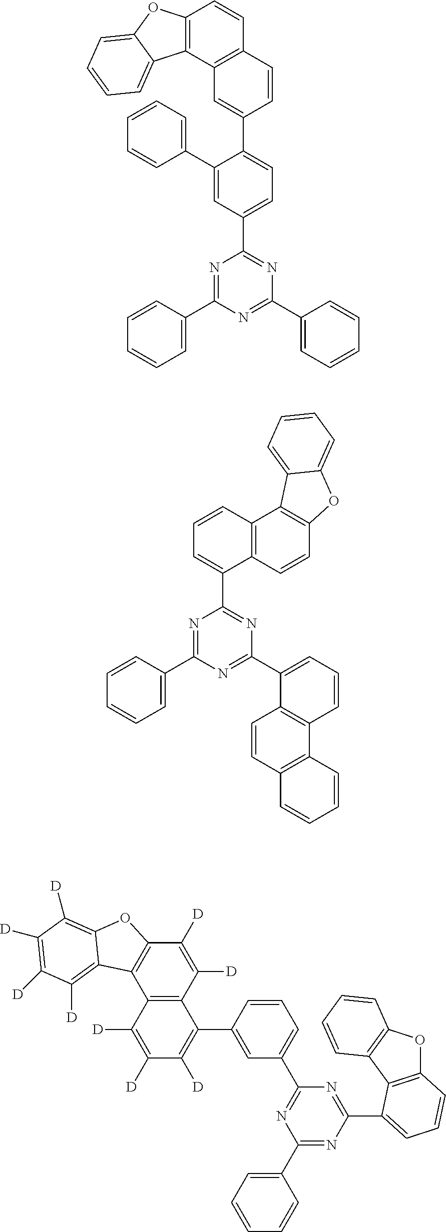 [SMILES: c1ccc(-c2nc(-c3ccccc3)nc(-c3ccc(-c4ccc5ccc6oc7ccccc7c6c5c4)c(-c4ccccc4)c3)n2)cc1 ; c1ccc(-c2nc(-c3cccc4c3ccc3ccccc34)nc(-c3cccc4c3ccc3oc5ccccc5c34)n2)cc1 ; [2H]c1c([2H])c([2H])c2c(oc3c([2H])c([2H])c4c(-c5cccc(-c6nc(-c7ccccc7)nc(-c7cccc8oc9ccccc9c78)n6)c5)c([2H])c([2H])c([2H])c4c32)c1[2H]]

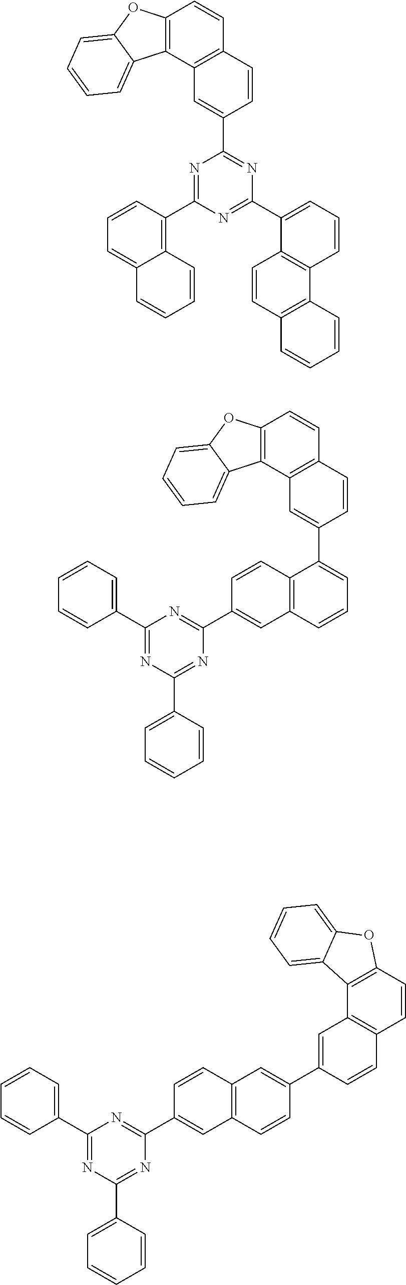 [SMILES: c1ccc2c(-c3nc(-c4ccc5ccc6oc7ccccc7c6c5c4)nc(-c4cccc5c4ccc4ccccc45)n3)cccc2c1 ; c1ccc(-c2nc(-c3ccccc3)nc(-c3ccc4c(-c5ccc6ccc7oc8ccccc8c7c6c5)cccc4c3)n2)cc1 ; c1ccc(-c2nc(-c3ccccc3)nc(-c3ccc4cc(-c5ccc6ccc7oc8ccccc8c7c6c5)ccc4c3)n2)cc1]

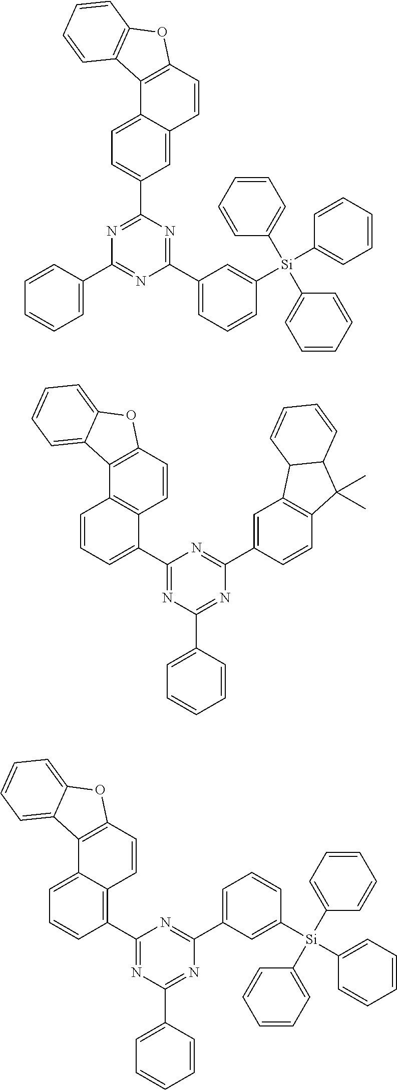 [SMILES: c1ccc(-c2nc(-c3cccc([Si](c4ccccc4)(c4ccccc4)c4ccccc4)c3)nc(-c3ccc4c(ccc5oc6ccccc6c54)c3)n2)cc1 ; CC1(C)c2ccc(-c3nc(-c4ccccc4)nc(-c4cccc5c4ccc4oc6ccccc6c45)n3)cc2C2C=CC=CC21 ; c1ccc(-c2nc(-c3cccc([Si](c4ccccc4)(c4ccccc4)c4ccccc4)c3)nc(-c3cccc4c3ccc3oc5ccccc5c34)n2)cc1]

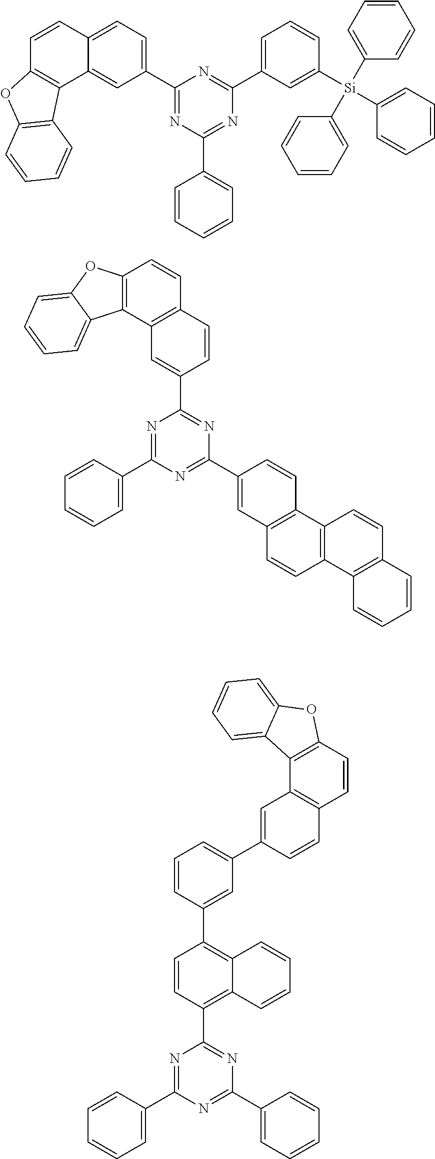

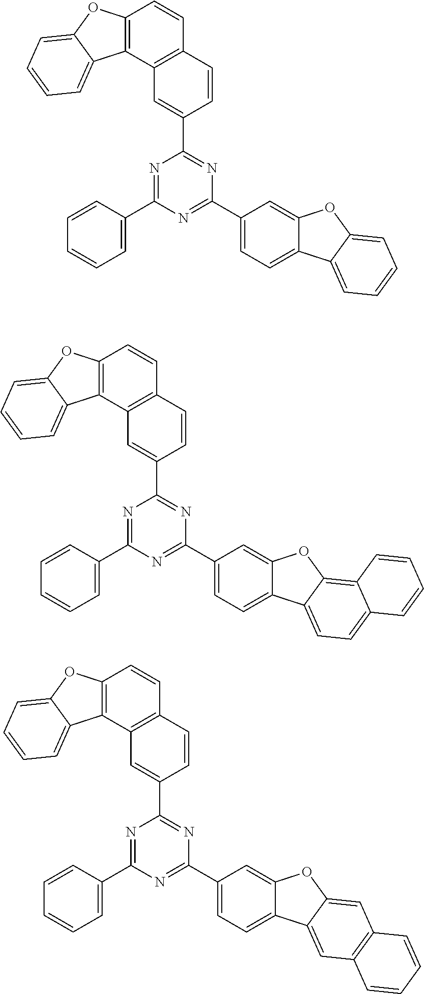 [SMILES: c1ccc(-c2nc(-c3ccc4c(c3)oc3ccccc34)nc(-c3ccc4ccc5oc6ccccc6c5c4c3)n2)cc1 ; c1ccc(-c2nc(-c3ccc4c(c3)oc3c5ccccc5ccc43)nc(-c3ccc4ccc5oc6ccccc6c5c4c3)n2)cc1 ; c1ccc(-c2nc(-c3ccc4c(c3)oc3cc5ccccc5cc34)nc(-c3ccc4ccc5oc6ccccc6c5c4c3)n2)cc1]

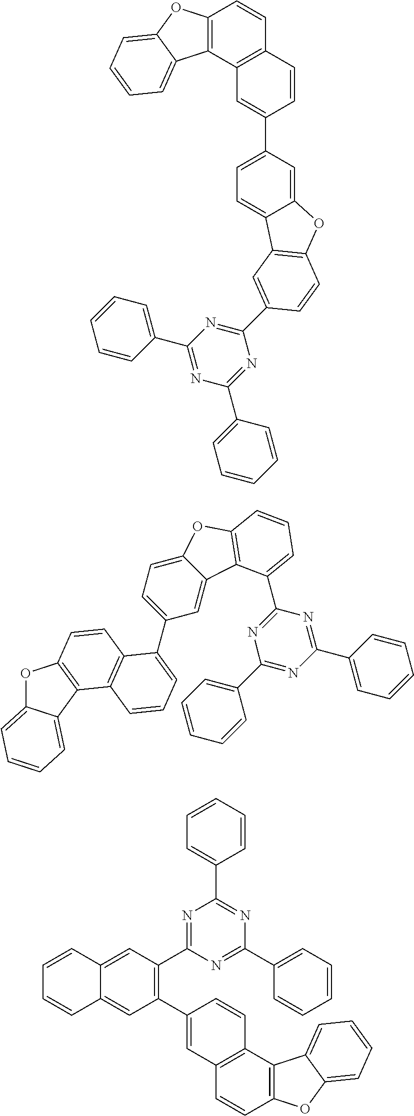

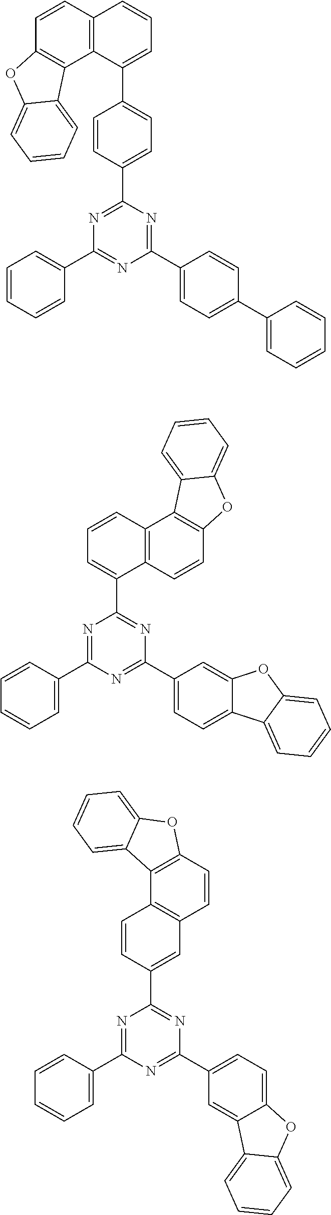 [SMILES: c1ccc(-c2ccc(-c3nc(-c4ccccc4)nc(-c4ccc(-c5cccc6ccc7oc8ccccc8c7c56)cc4)n3)cc2)cc1 ; c1ccc(-c2nc(-c3ccc4c(c3)oc3ccccc34)nc(-c3cccc4c3ccc3oc5ccccc5c34)n2)cc1 ; c1ccc(-c2nc(-c3ccc4c(ccc5oc6ccccc6c54)c3)nc(-c3ccc4oc5ccccc5c4c3)n2)cc1]

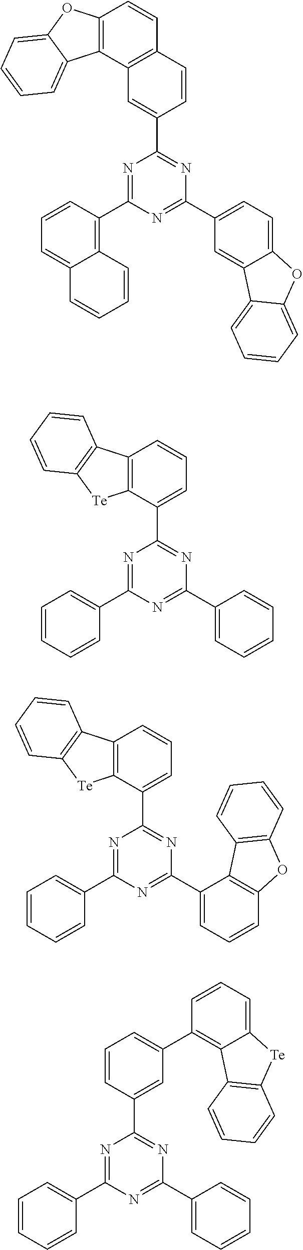

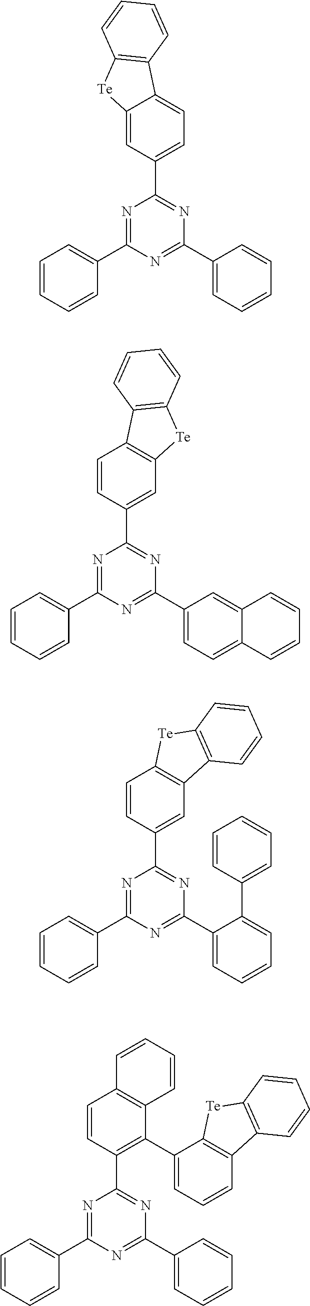

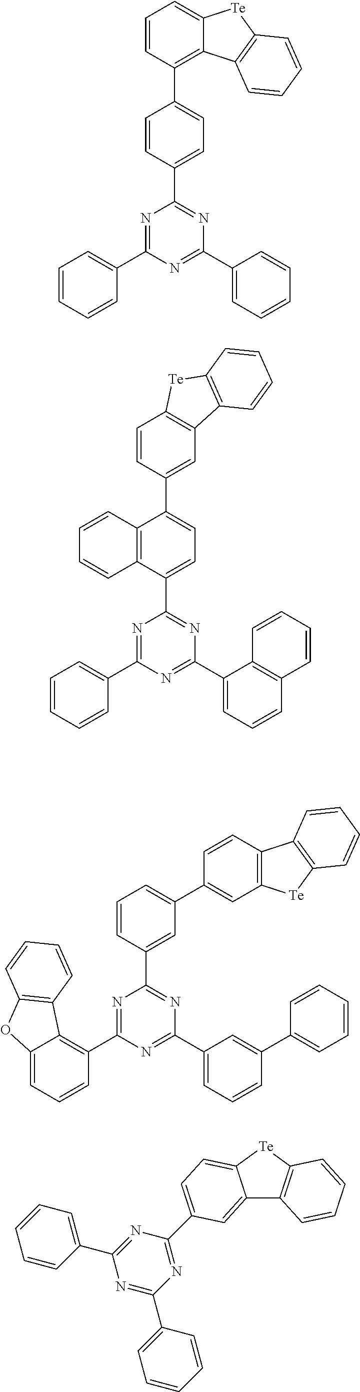

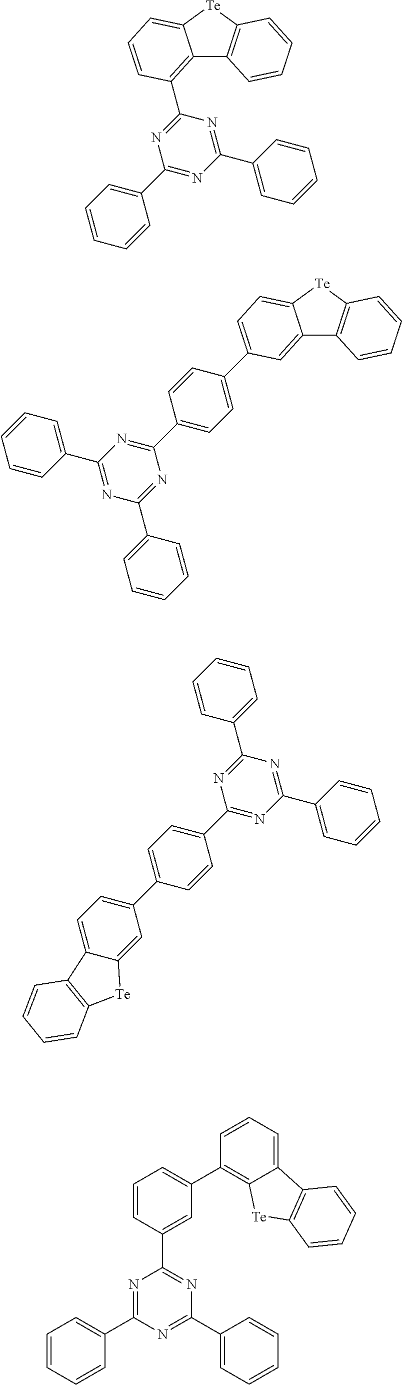

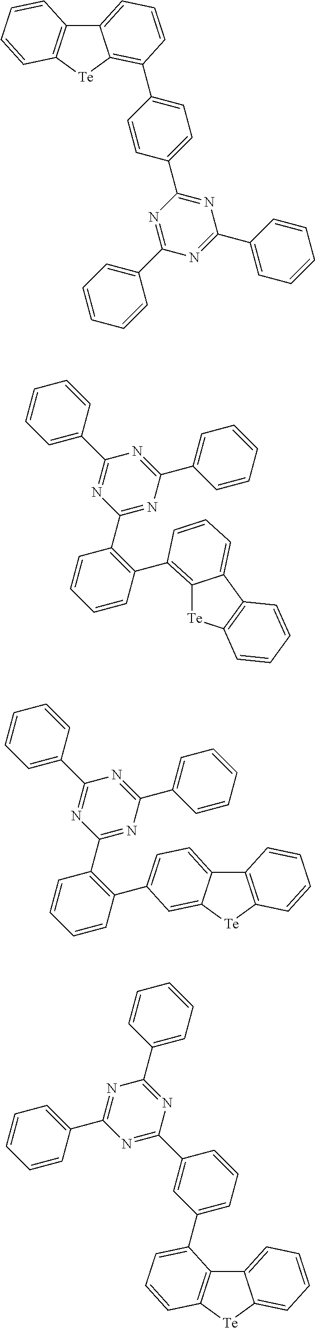

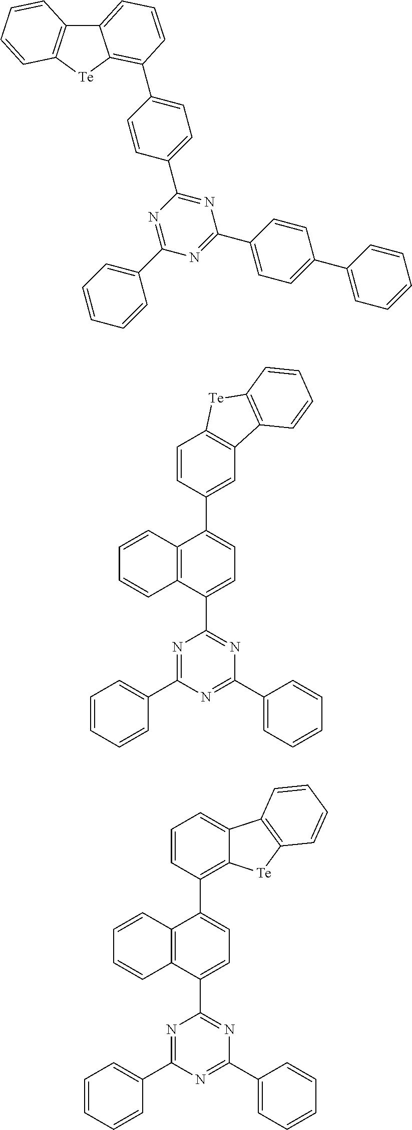

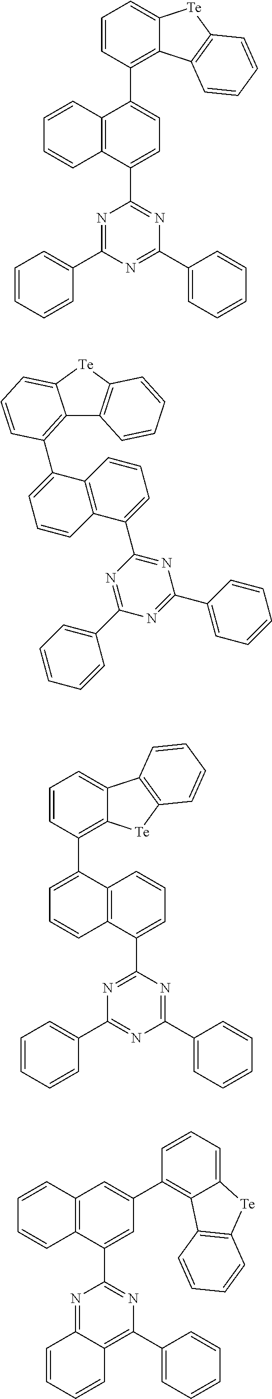

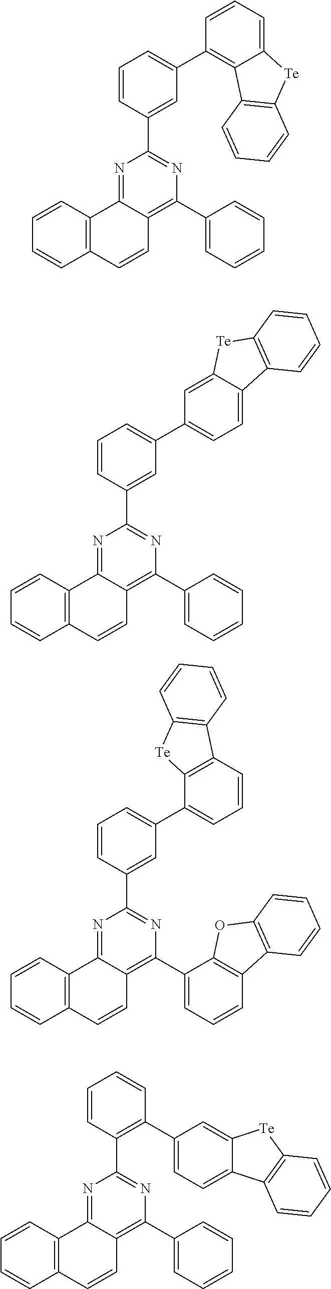

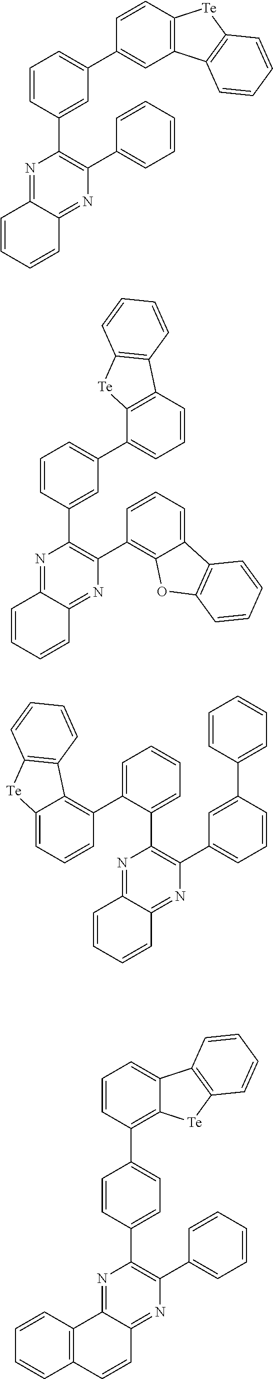

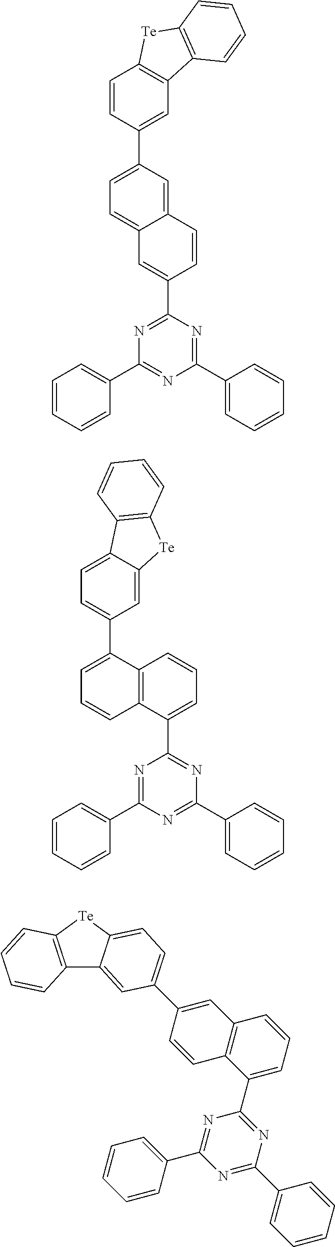

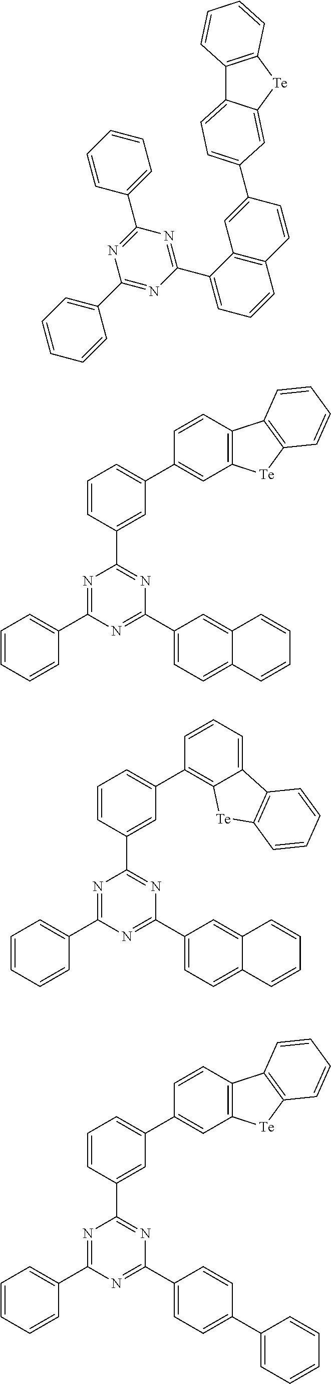

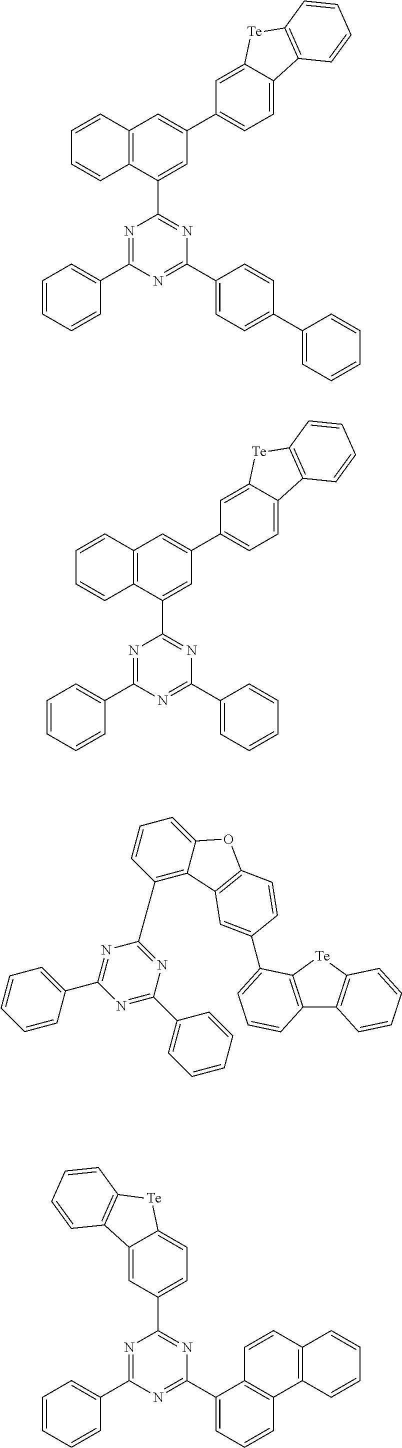

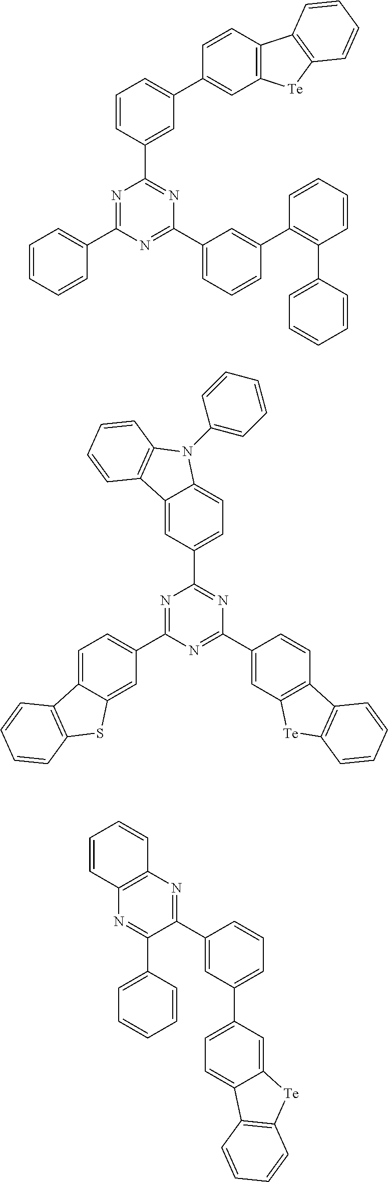

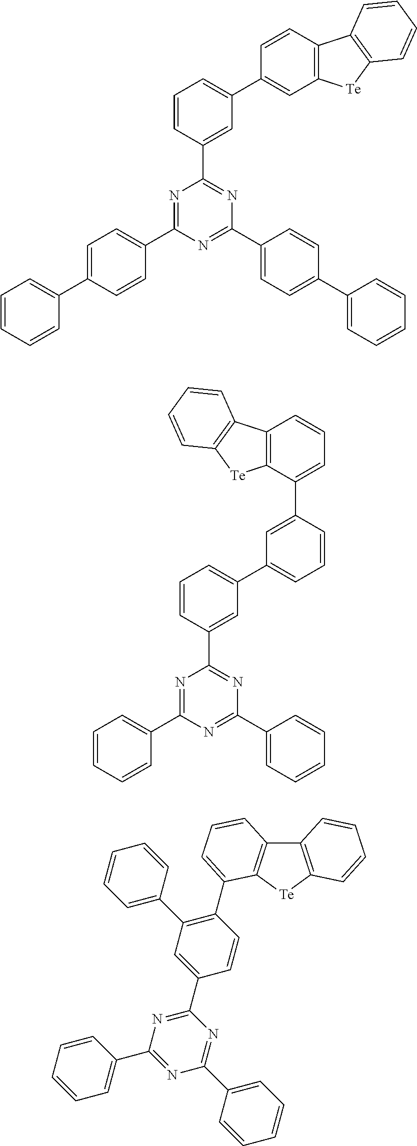

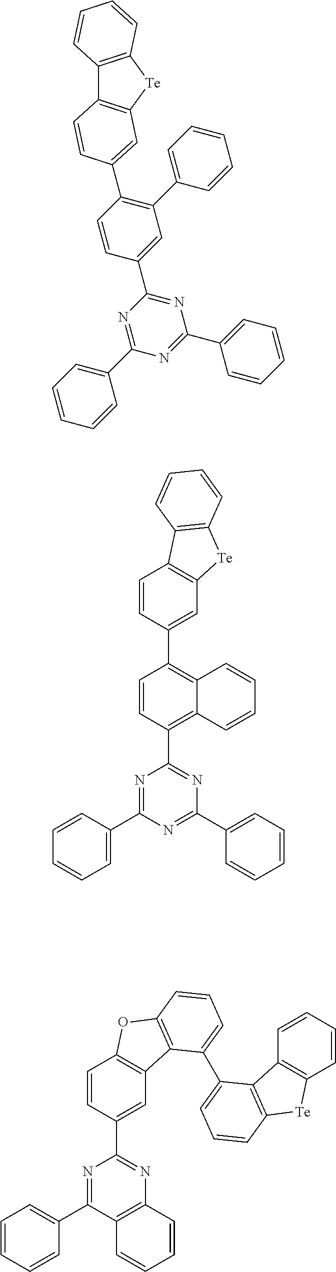

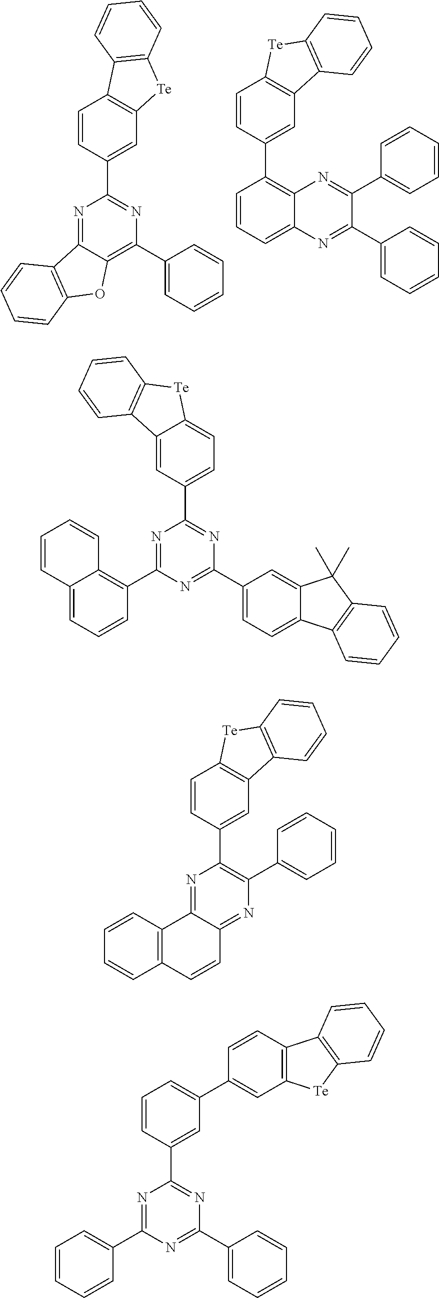

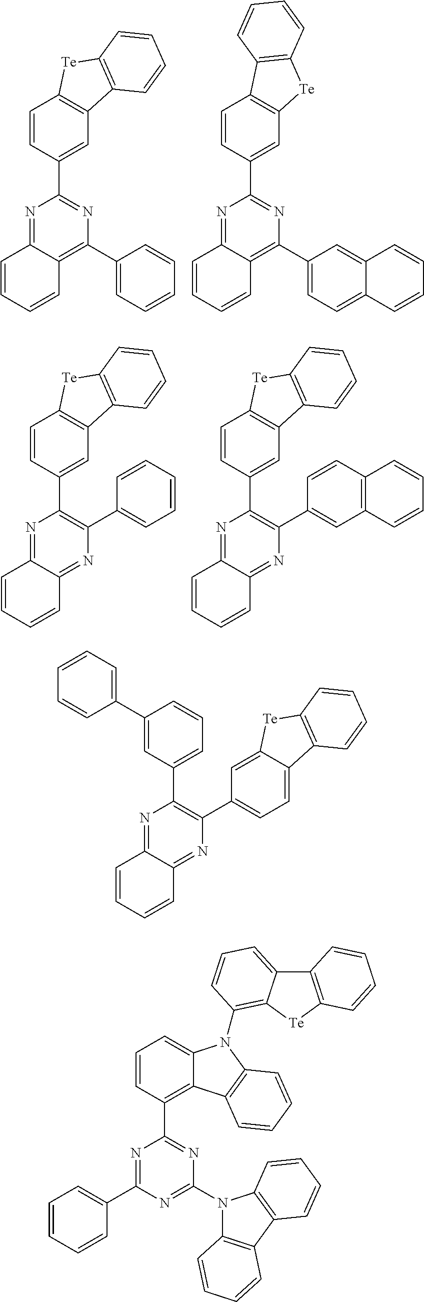

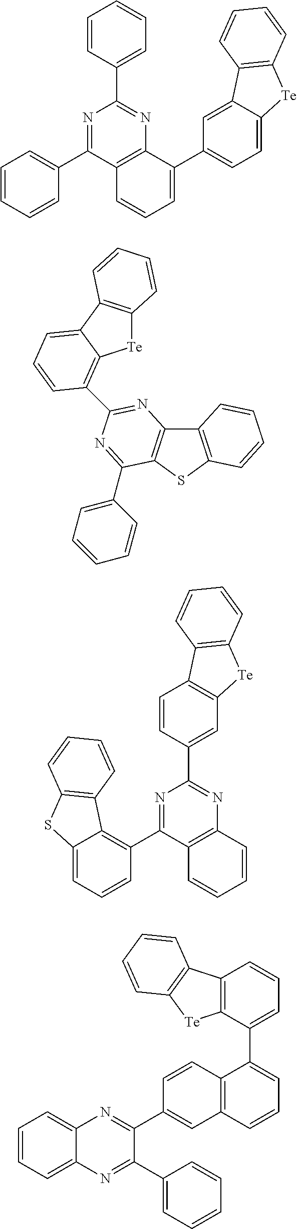

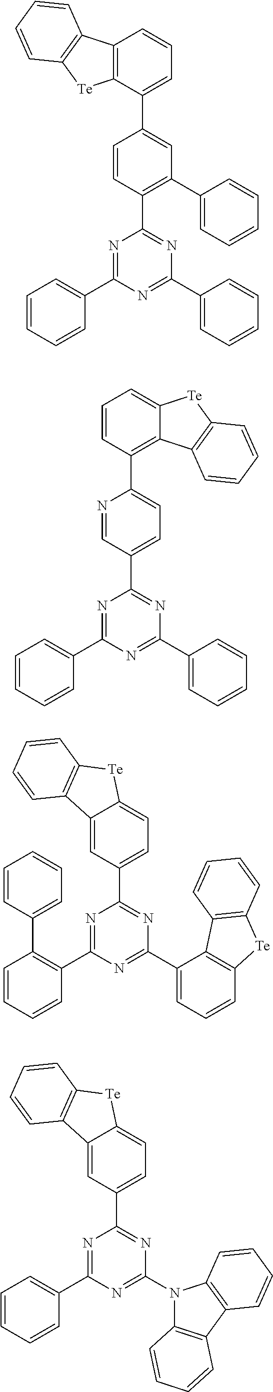

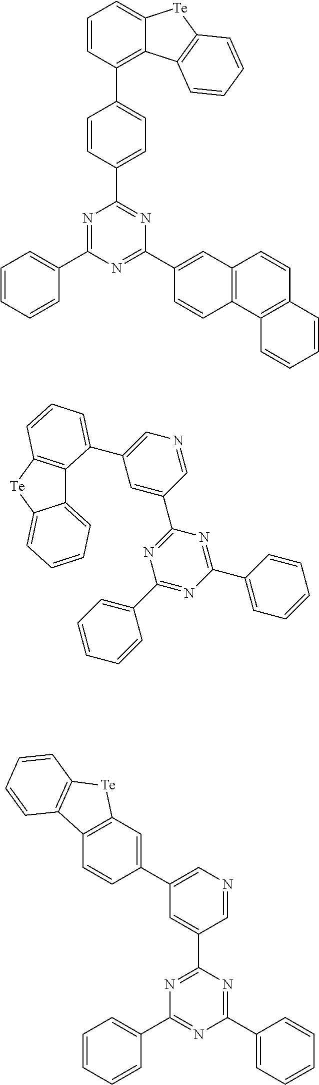

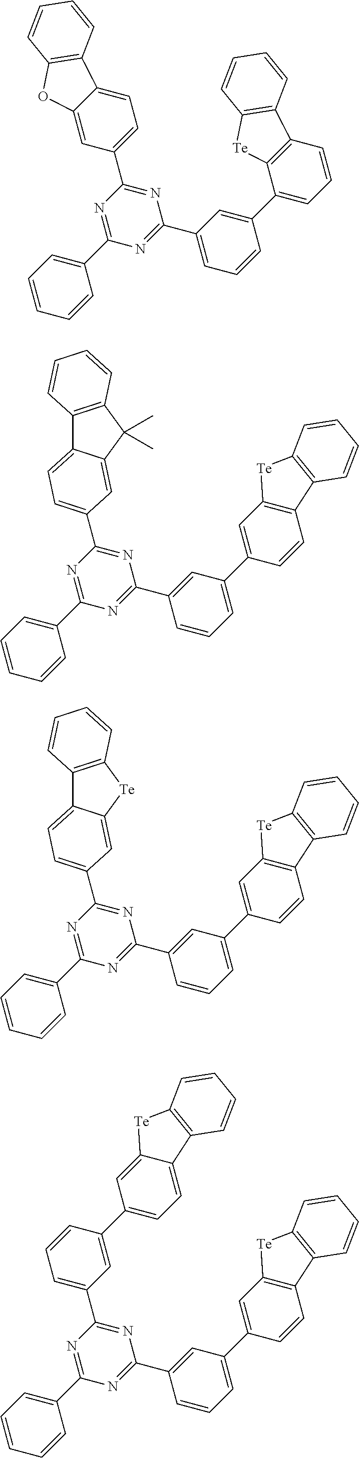

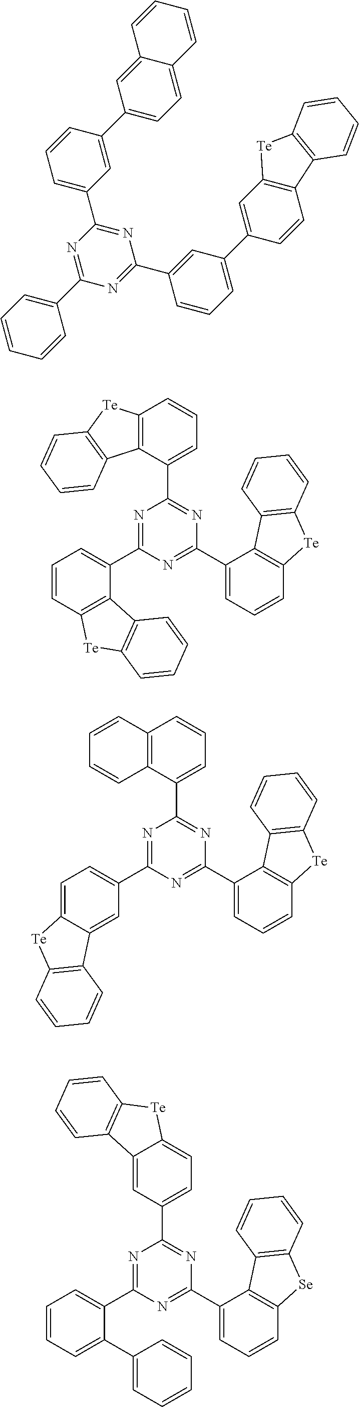

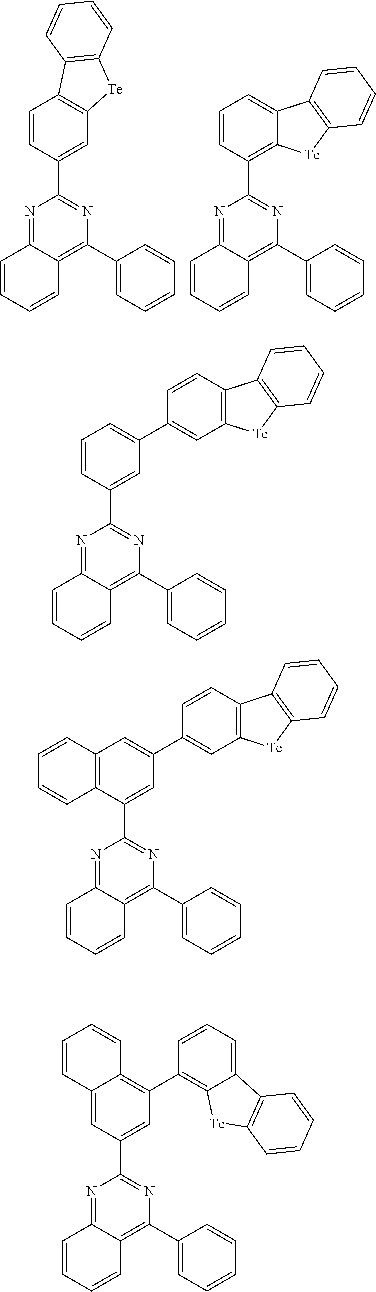

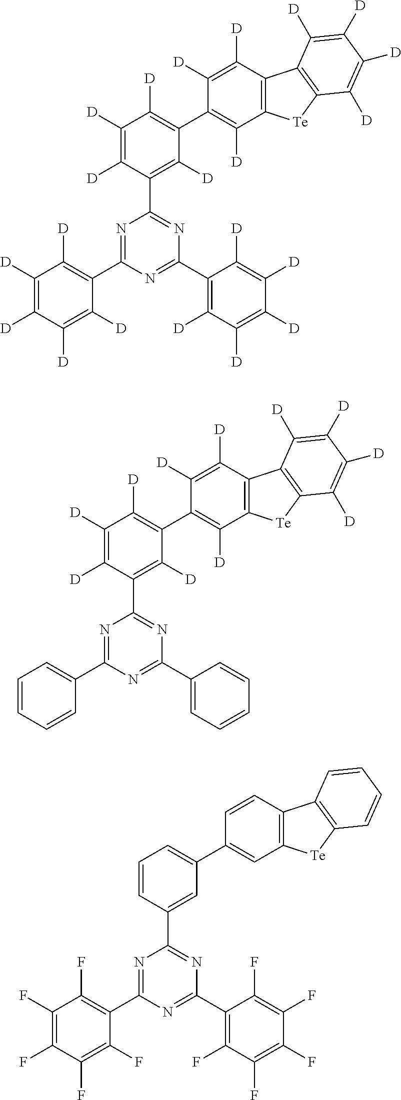

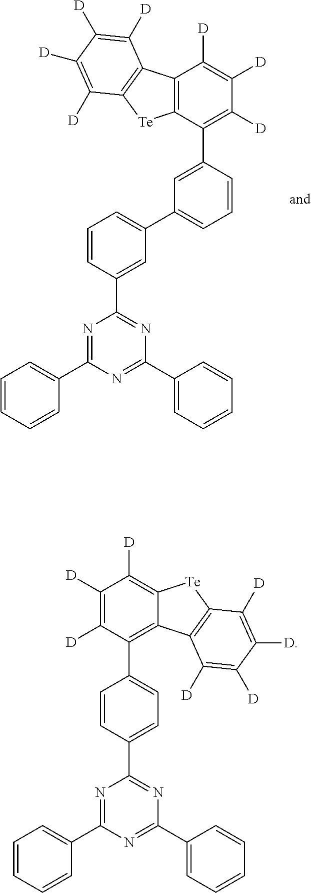

12. The organic electroluminescent material according to claim 8, wherein the compound represented by formula 15 is selected from the group consisting of the following compounds:

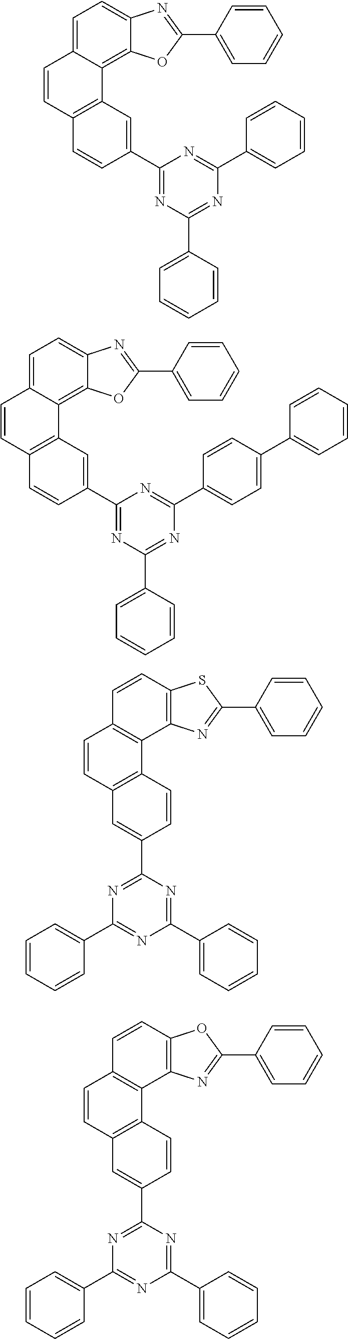

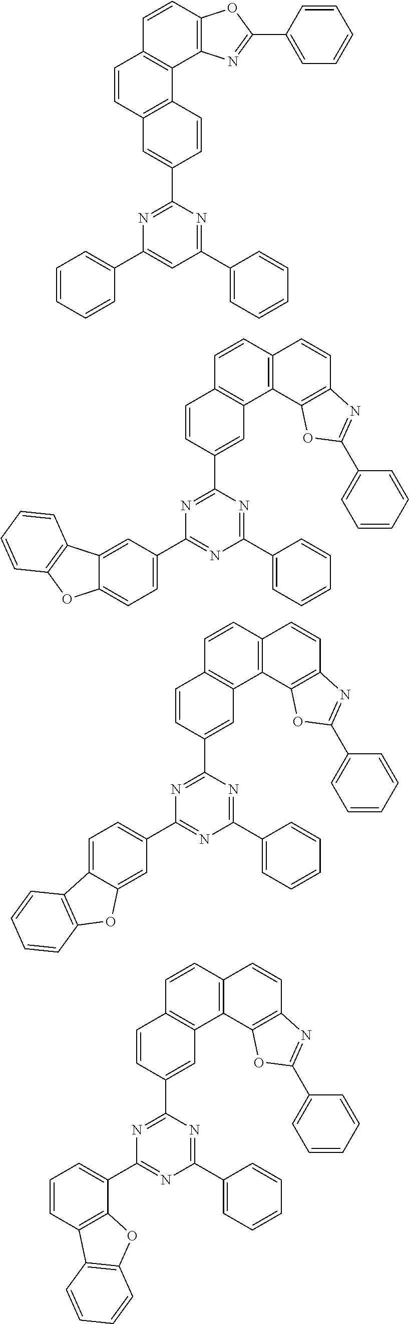

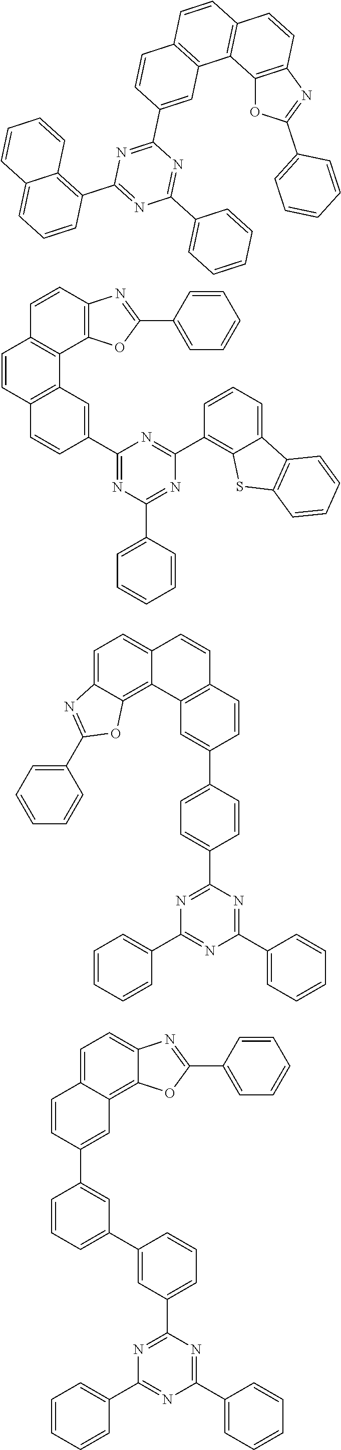

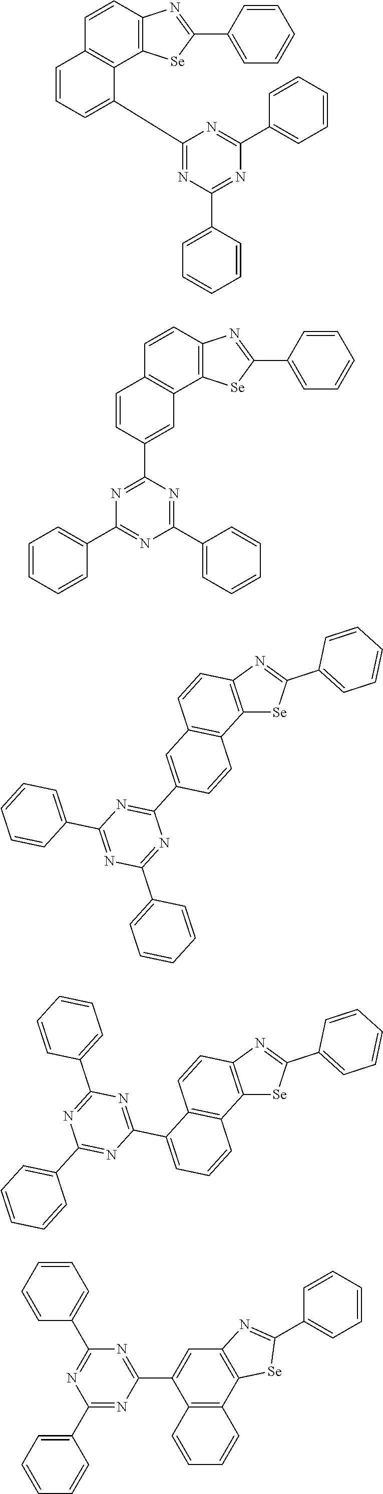

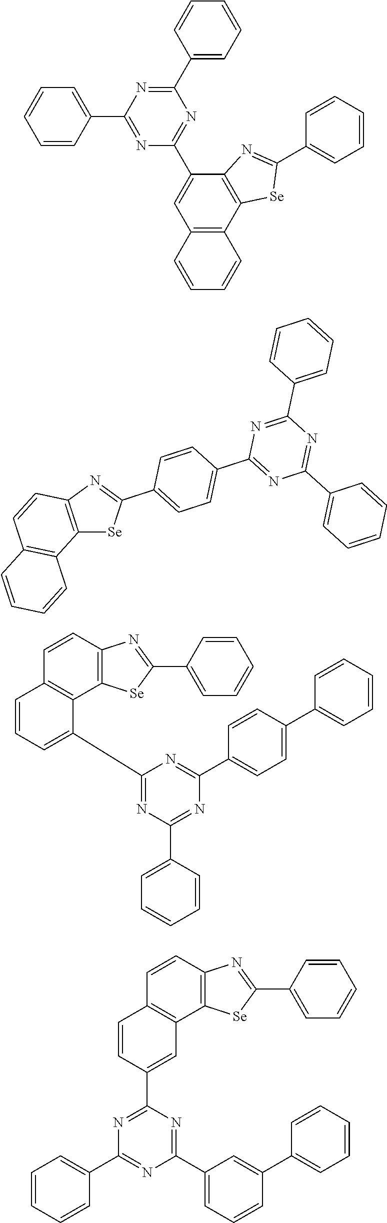

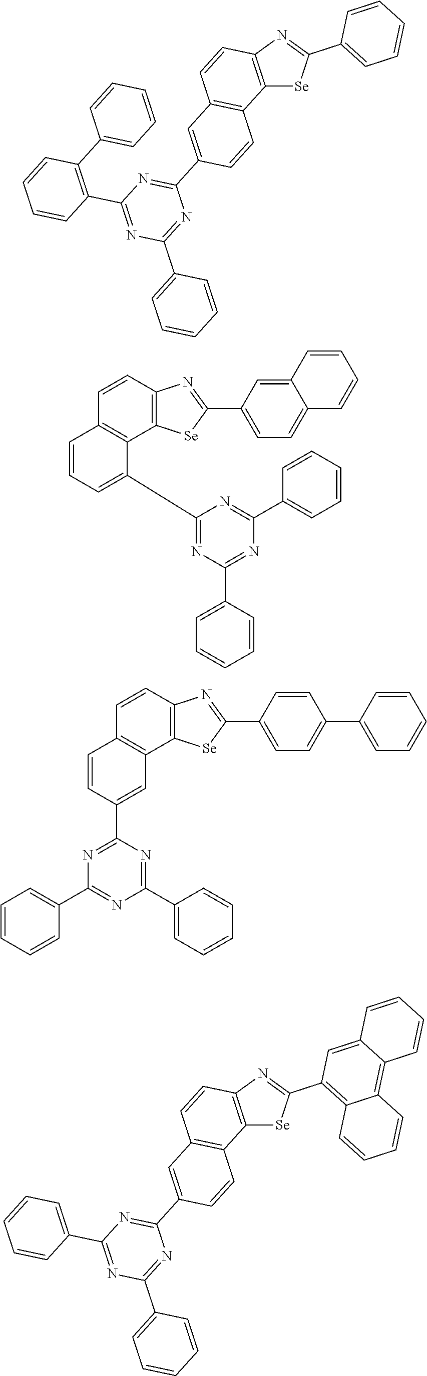

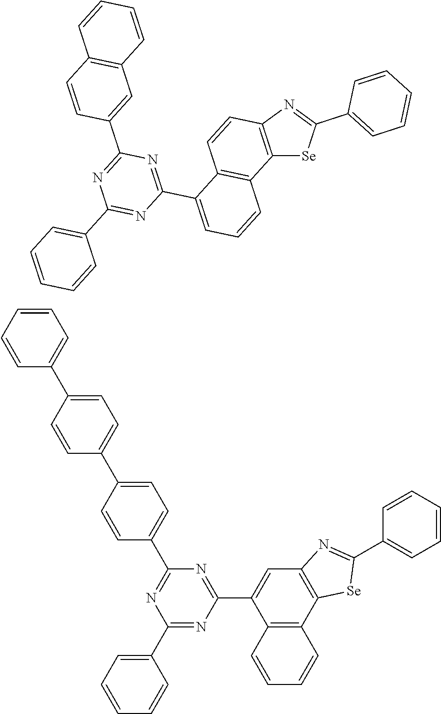

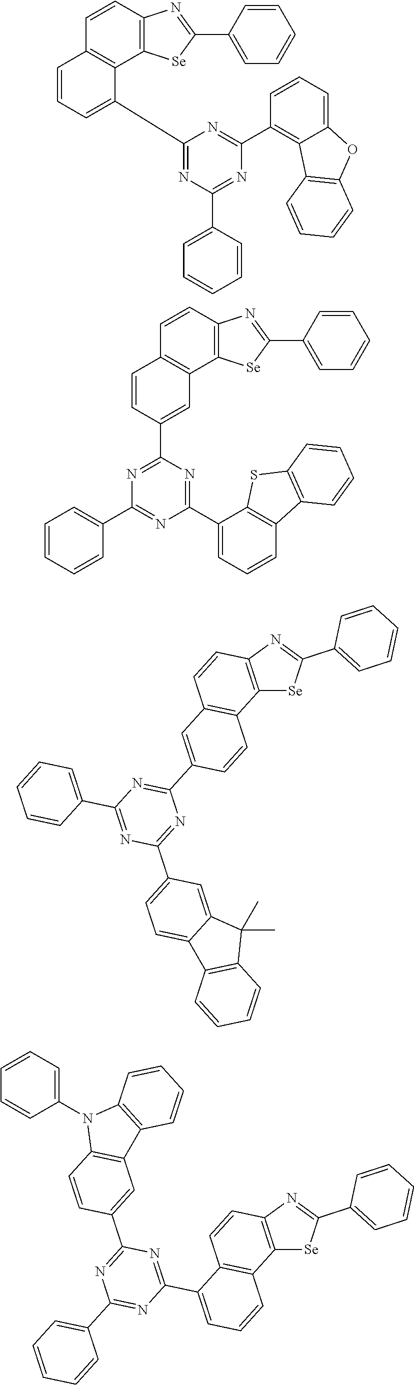

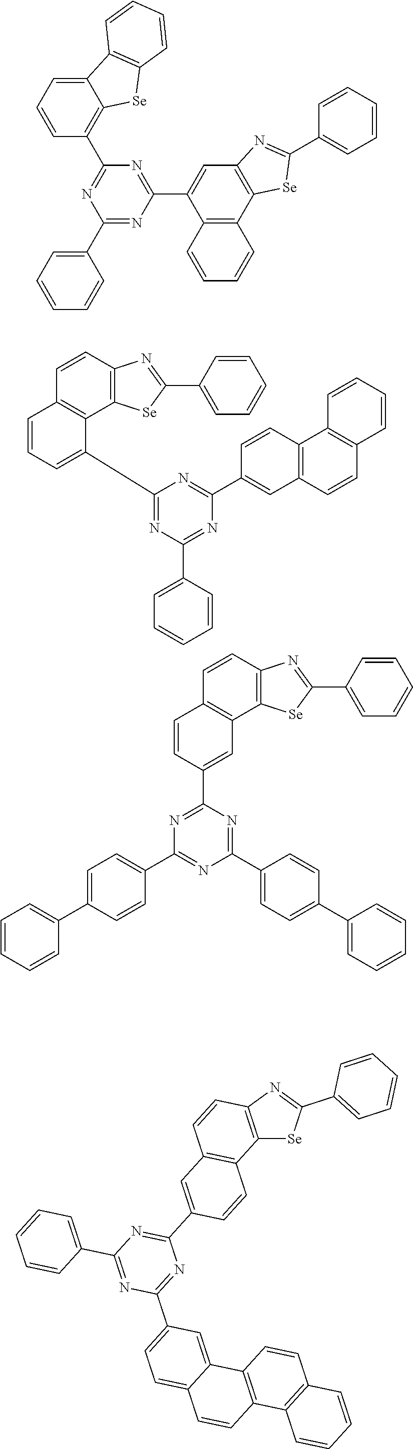

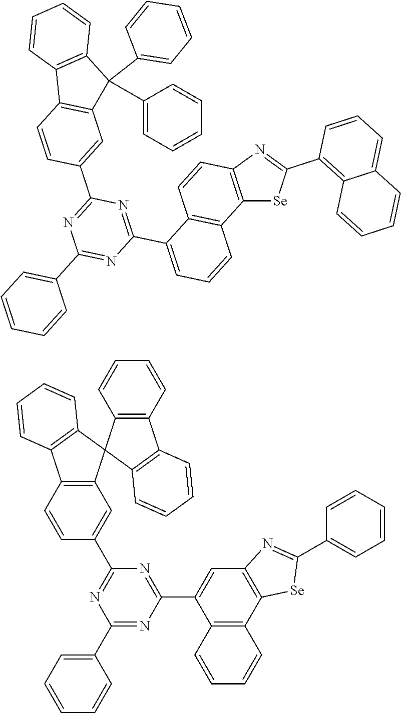 [SMILES: c1ccc(-c2nc(-c3ccc4c(c3)C(c3ccccc3)(c3ccccc3)c3ccccc3-4)nc(-c3cccc4c3ccc3nc(-c5cccc6ccccc56)[se]c34)n2)cc1 ; c1ccc(-c2nc(-c3ccc4c(c3)C3(c5ccccc5-c5ccccc53)c3ccccc3-4)nc(-c3cc4nc(-c5ccccc5)[se]c4c4ccccc34)n2)cc1]

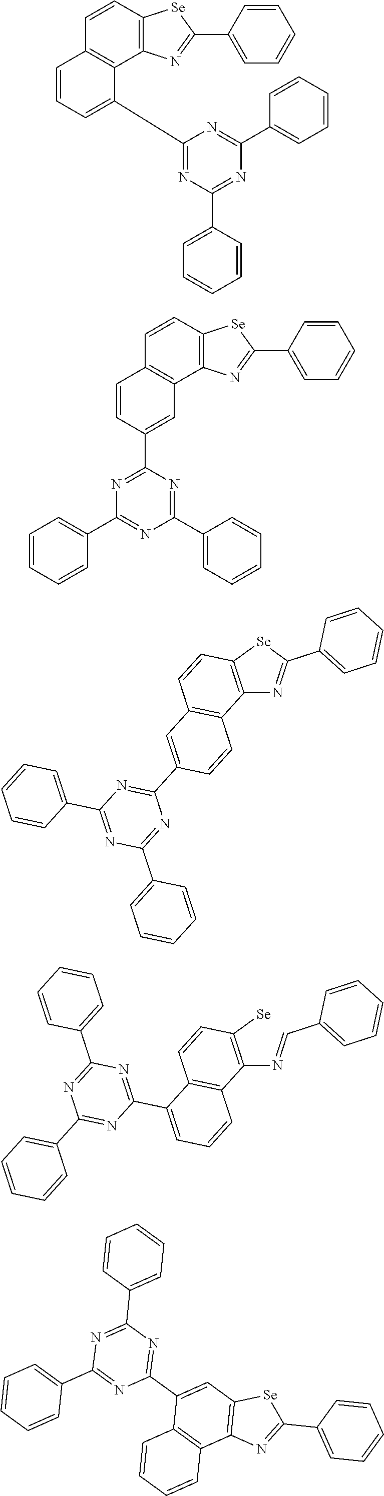

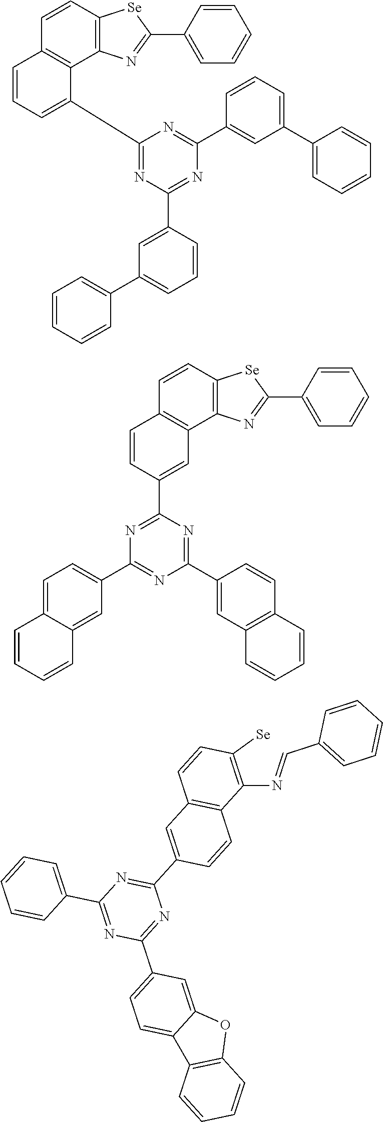

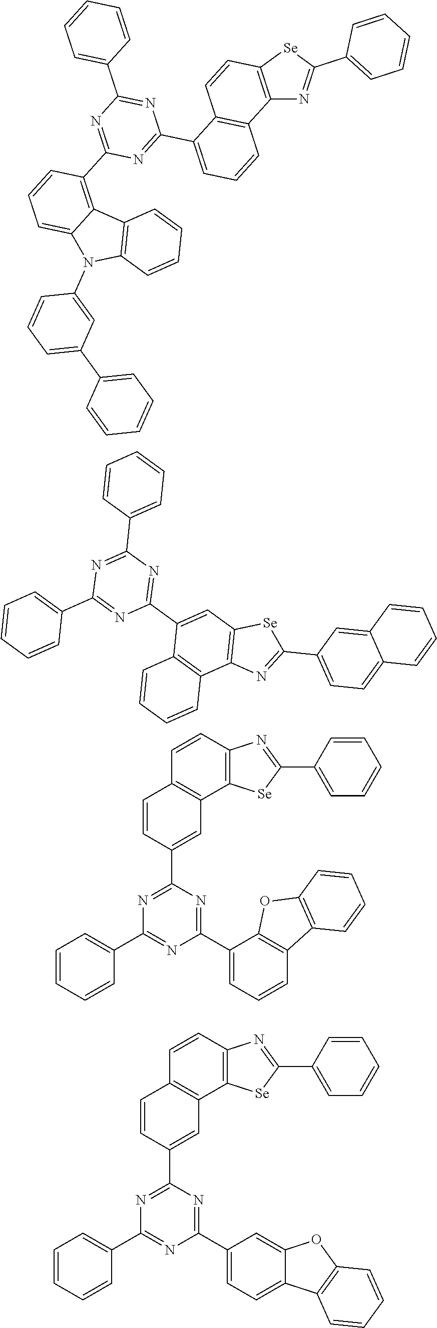

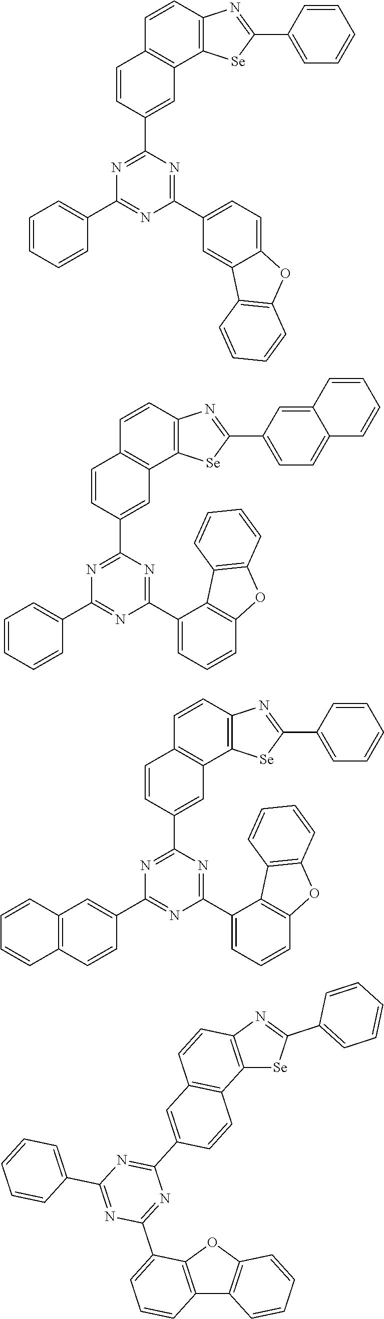

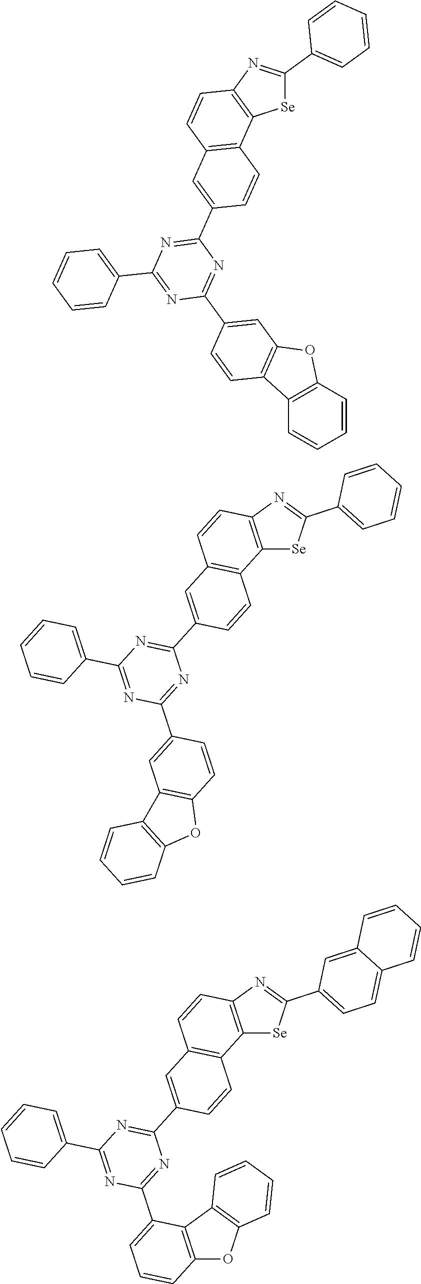

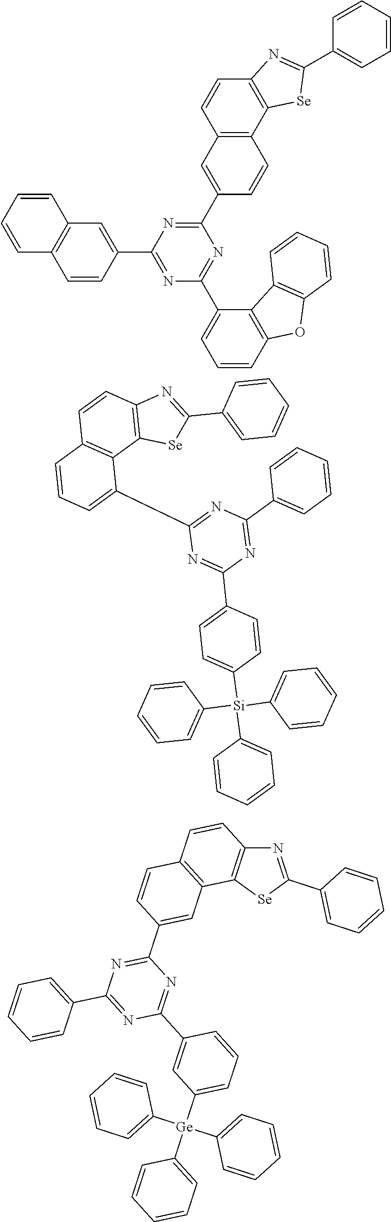

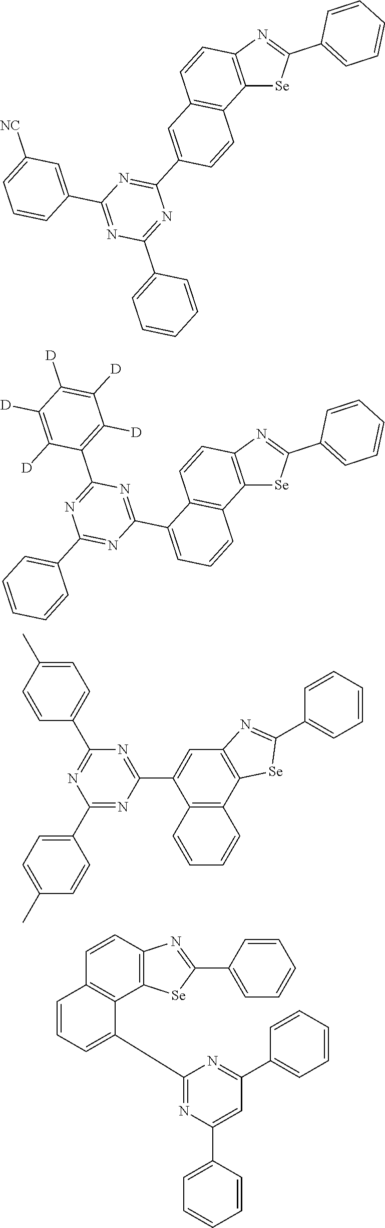

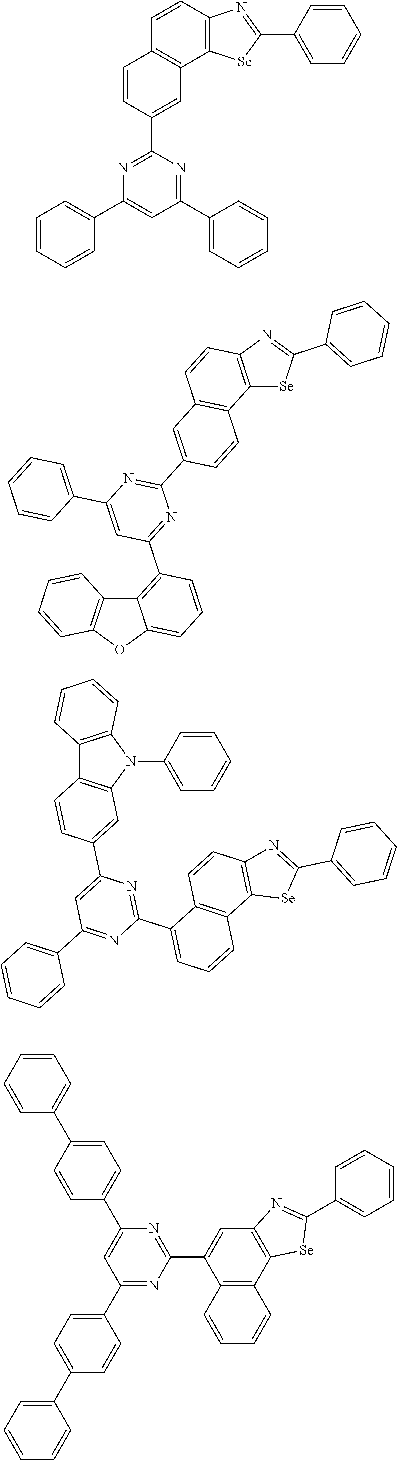

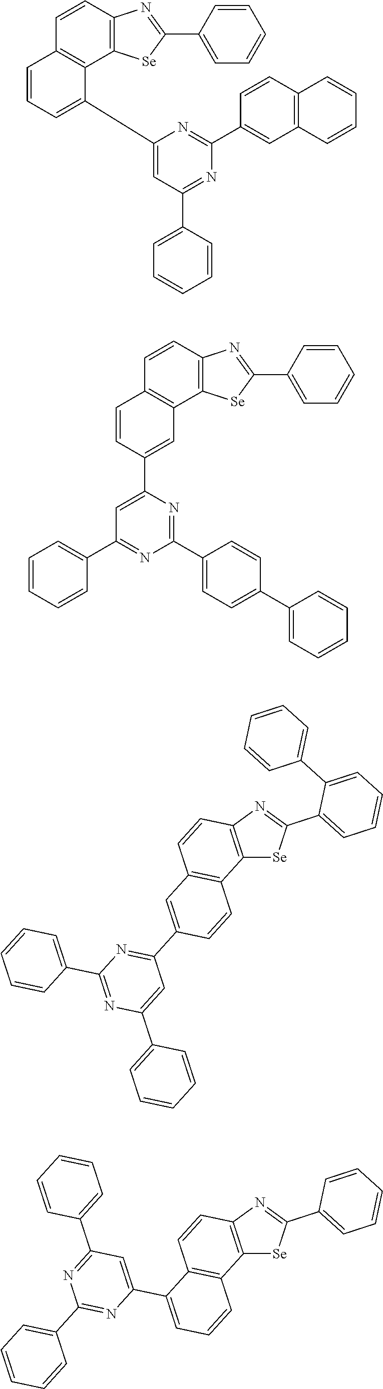 [SMILES: c1ccc(-c2cc(-c3cccc4ccc5nc(-c6ccccc6)[se]c5c34)nc(-c3ccc4ccccc4c3)n2)cc1 ; c1ccc(-c2ccc(-c3nc(-c4ccccc4)cc(-c4ccc5ccc6nc(-c7ccccc7)[se]c6c5c4)n3)cc2)cc1 ; c1ccc(-c2cc(-c3ccc4c(ccc5nc(-c6ccccc6-c6ccccc6)[se]c54)c3)nc(-c3ccccc3)n2)cc1 ; c1ccc(-c2cc(-c3cccc4c3ccc3nc(-c5ccccc5)[se]c34)nc(-c3ccccc3)n2)cc1]

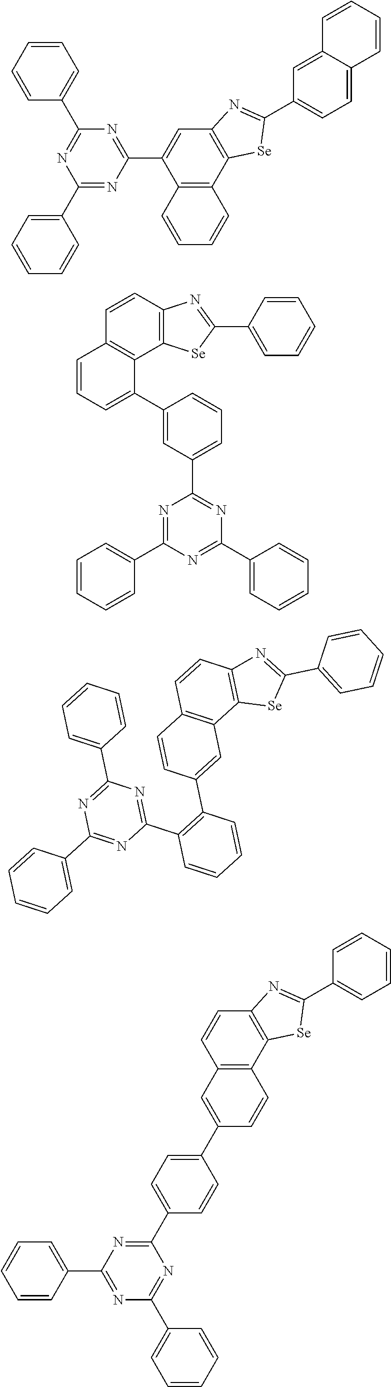 [SMILES: c1ccc(-c2nc(-c3ccccc3)nc(-c3cc4nc(-c5ccc6ccccc6c5)[se]c4c4ccccc34)n2)cc1 ; c1ccc(-c2nc(-c3ccccc3)nc(-c3cccc(-c4cccc5ccc6nc(-c7ccccc7)[se]c6c45)c3)n2)cc1 ; c1ccc(-c2nc(-c3ccccc3)nc(-c3ccccc3-c3ccc4ccc5nc(-c6ccccc6)[se]c5c4c3)n2)cc1 ; c1ccc(-c2nc(-c3ccccc3)nc(-c3ccc(-c4ccc5c(ccc6nc(-c7ccccc7)[se]c65)c4)cc3)n2)cc1]

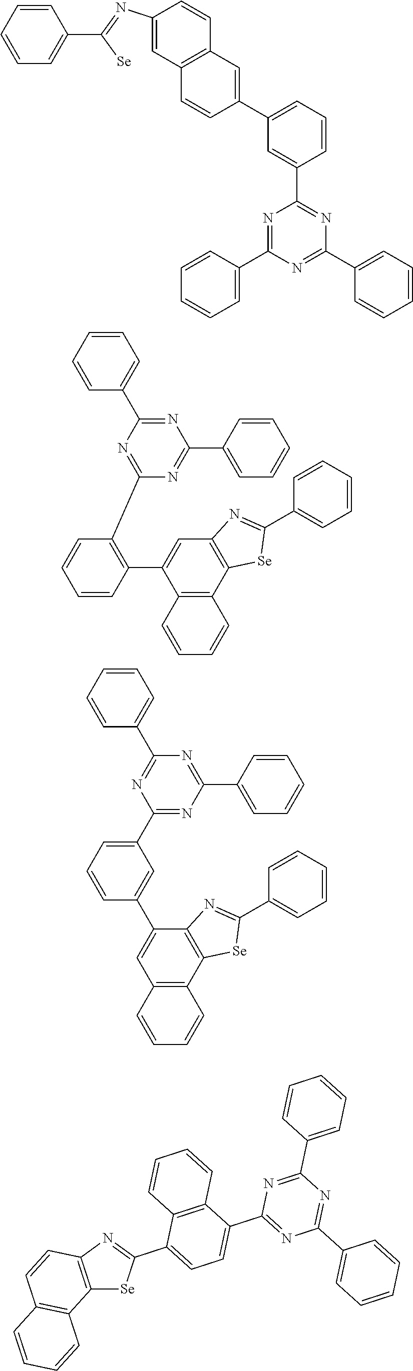

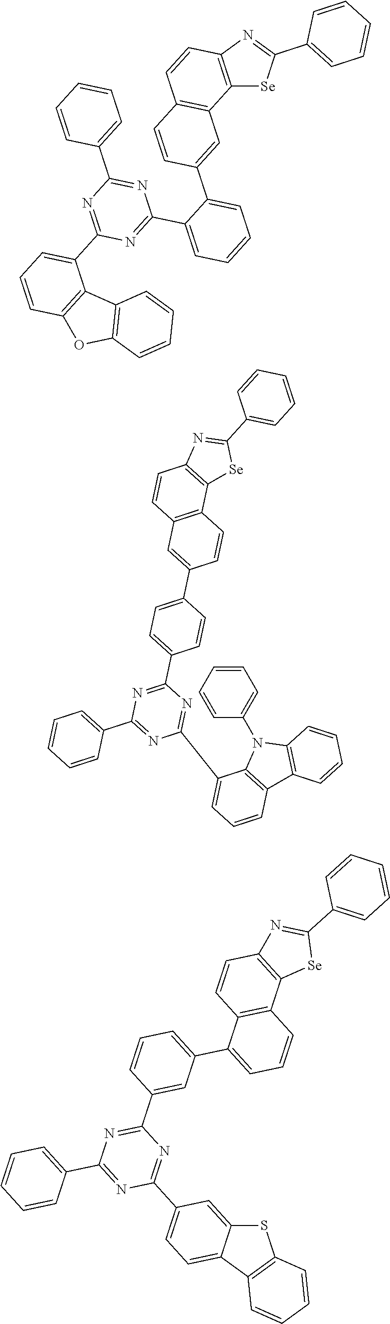 [SMILES: c1ccc(-c2nc(-c3ccccc3-c3ccc4ccc5nc(-c6ccccc6)[se]c5c4c3)nc(-c3cccc4oc5ccccc5c34)n2)cc1 ; c1ccc(-c2nc(-c3ccc(-c4ccc5c(ccc6nc(-c7ccccc7)[se]c65)c4)cc3)nc(-c3cccc4c5ccccc5n(-c5ccccc5)c34)n2)cc1 ; c1ccc(-c2nc(-c3cccc(-c4cccc5c4ccc4nc(-c6ccccc6)[se]c45)c3)nc(-c3ccc4c(c3)sc3ccccc34)n2)cc1]

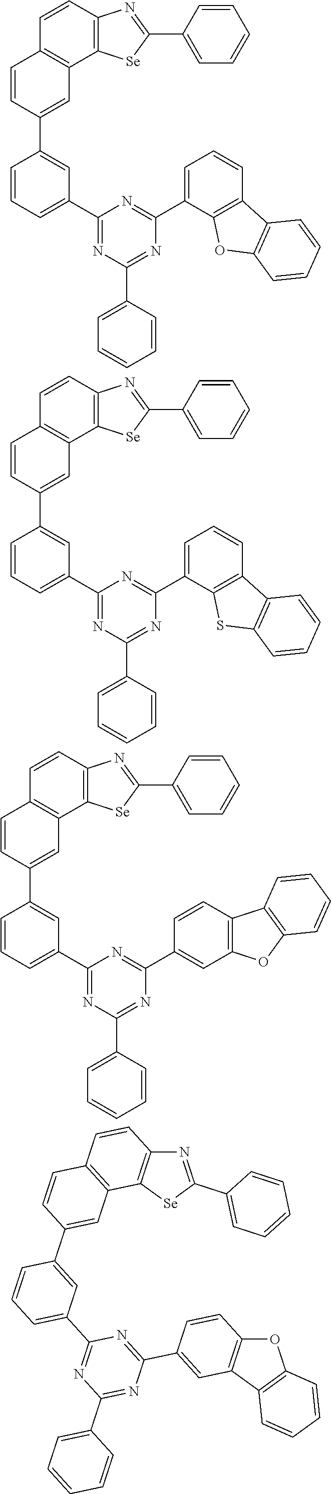

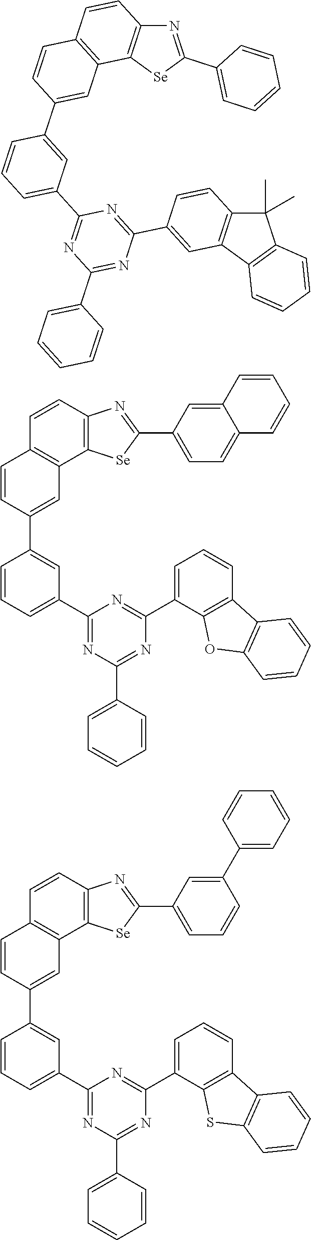

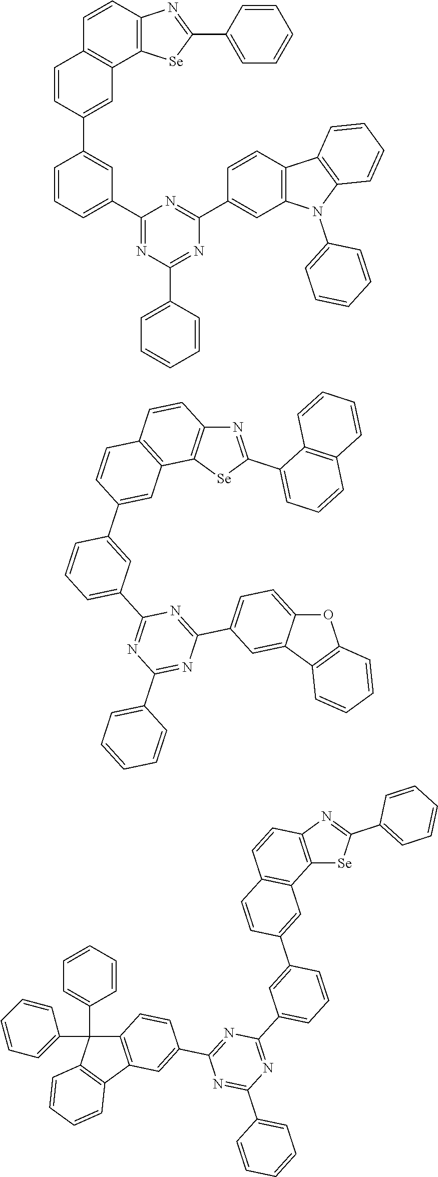

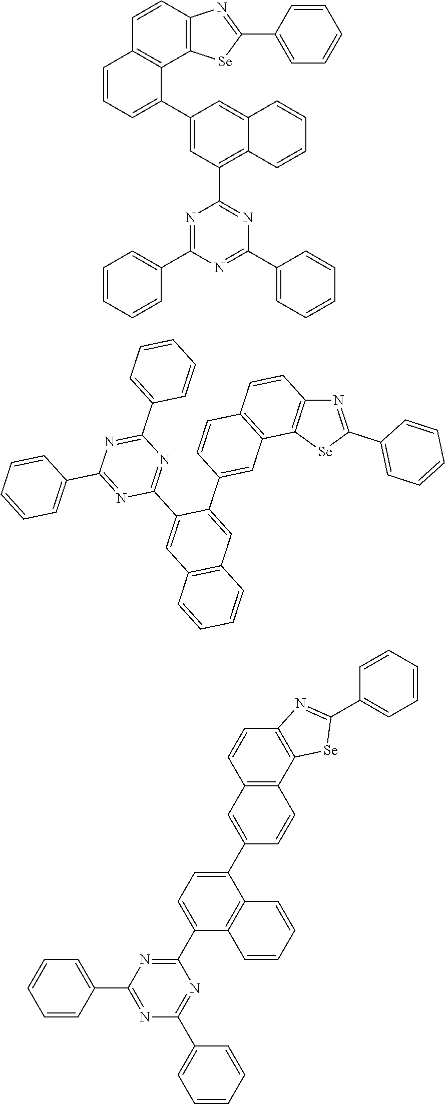

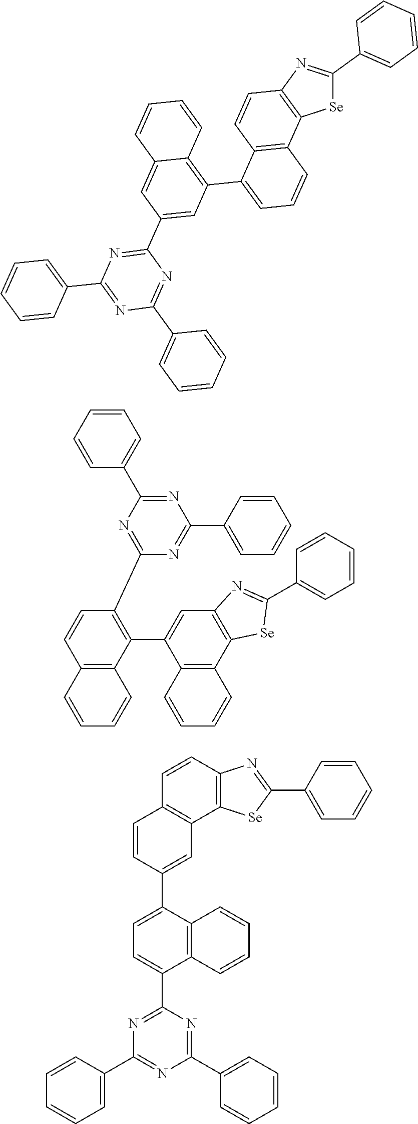

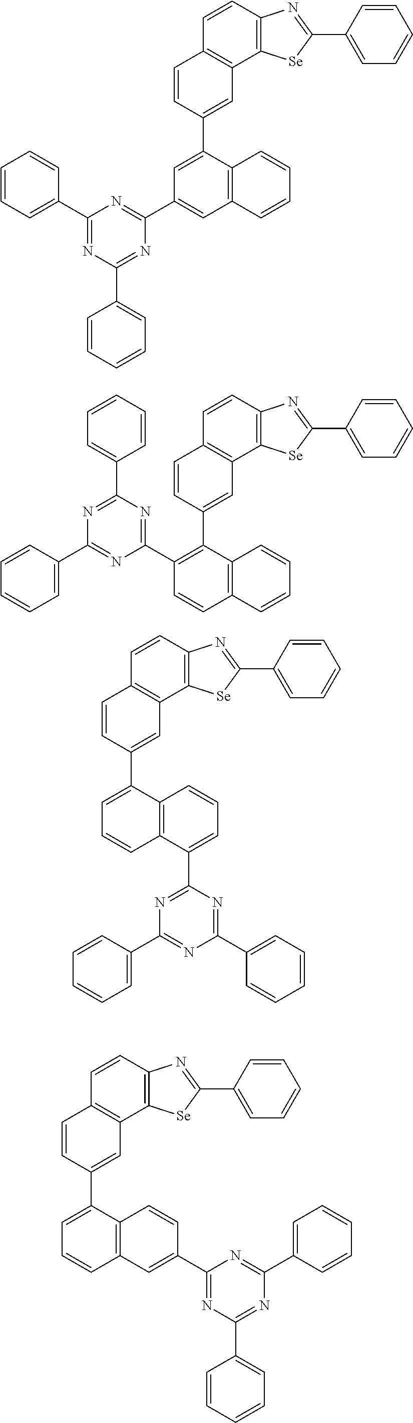

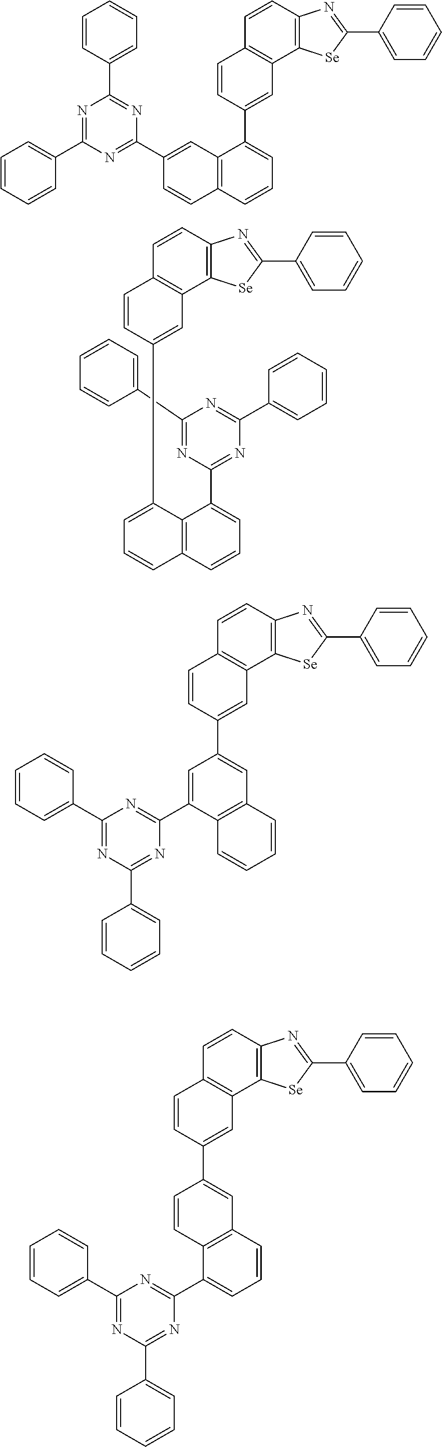

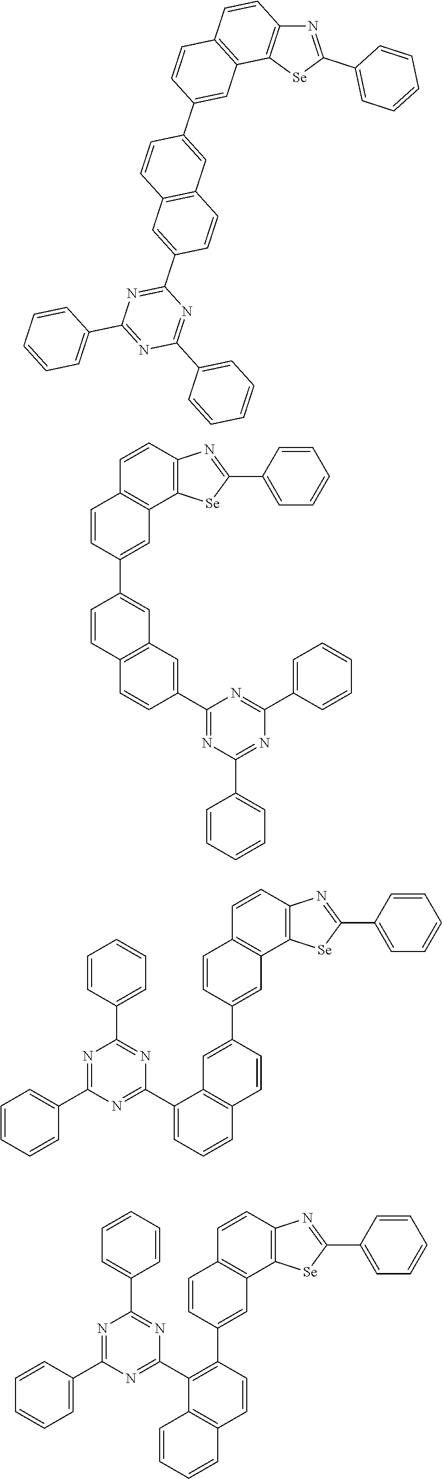

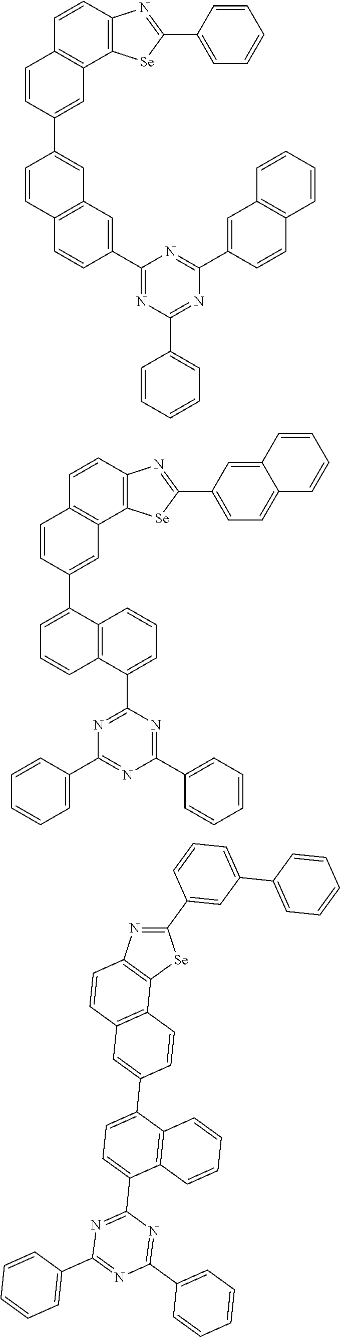

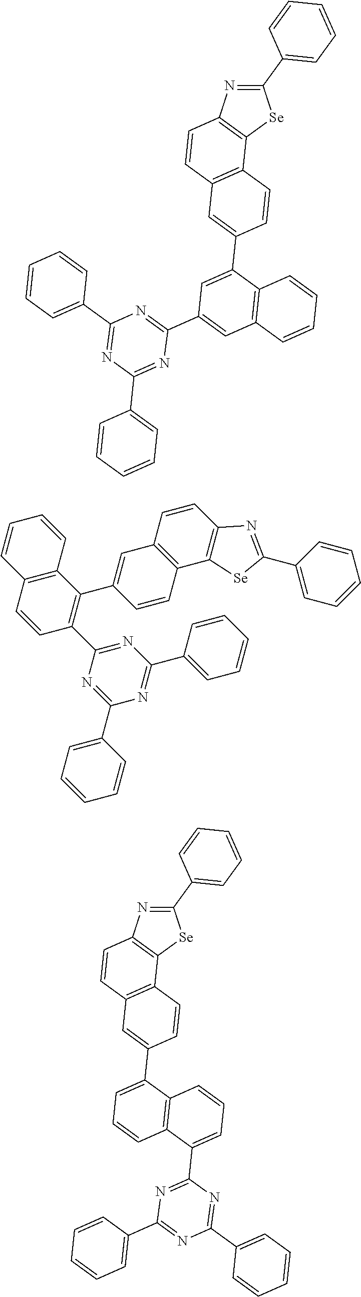 [SMILES: c1ccc(-c2nc(-c3ccccc3)nc(-c3cc(-c4ccc5c(ccc6nc(-c7ccccc7)[se]c65)c4)c4ccccc4c3)n2)cc1 ; c1ccc(-c2nc(-c3ccccc3)nc(-c3ccc4ccccc4c3-c3ccc4c(ccc5nc(-c6ccccc6)[se]c54)c3)n2)cc1 ; c1ccc(-c2nc(-c3ccccc3)nc(-c3cccc4c(-c5ccc6c(ccc7nc(-c8ccccc8)[se]c76)c5)cccc34)n2)cc1]

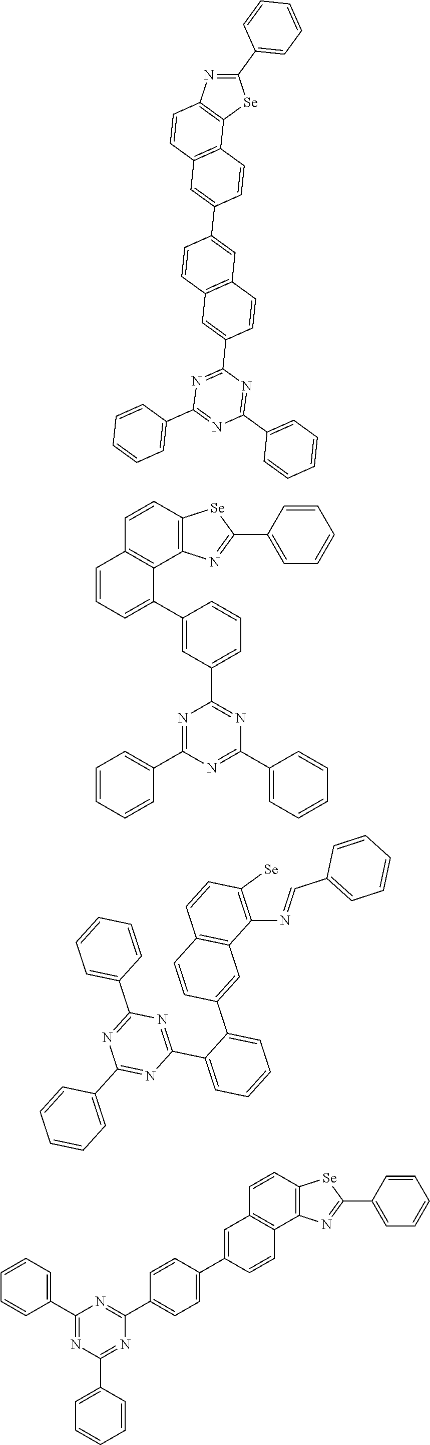

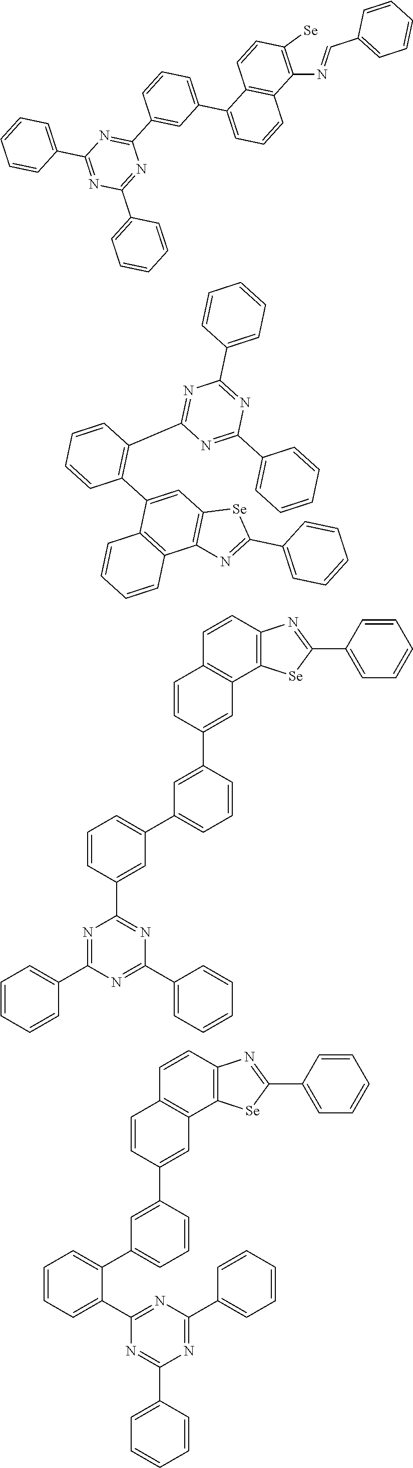

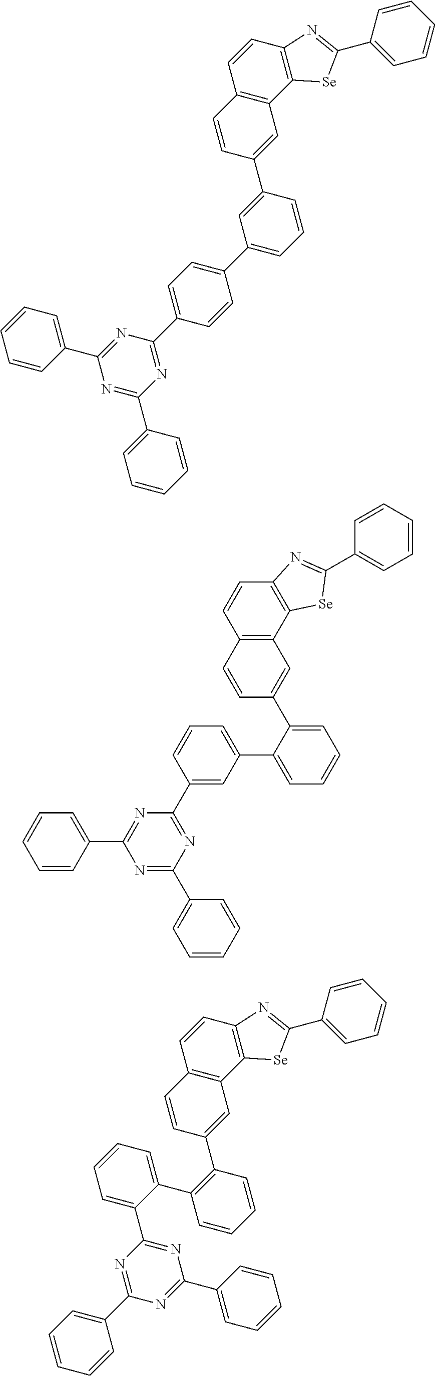 [SMILES: c1ccc(-c2nc(-c3ccccc3)nc(-c3ccc(-c4cccc(-c5ccc6ccc7nc(-c8ccccc8)[se]c7c6c5)c4)cc3)n2)cc1 ; c1ccc(-c2nc(-c3ccccc3)nc(-c3cccc(-c4ccccc4-c4ccc5ccc6nc(-c7ccccc7)[se]c6c5c4)c3)n2)cc1 ; c1ccc(-c2nc(-c3ccccc3)nc(-c3ccccc3-c3ccccc3-c3ccc4ccc5nc(-c6ccccc6)[se]c5c4c3)n2)cc1]

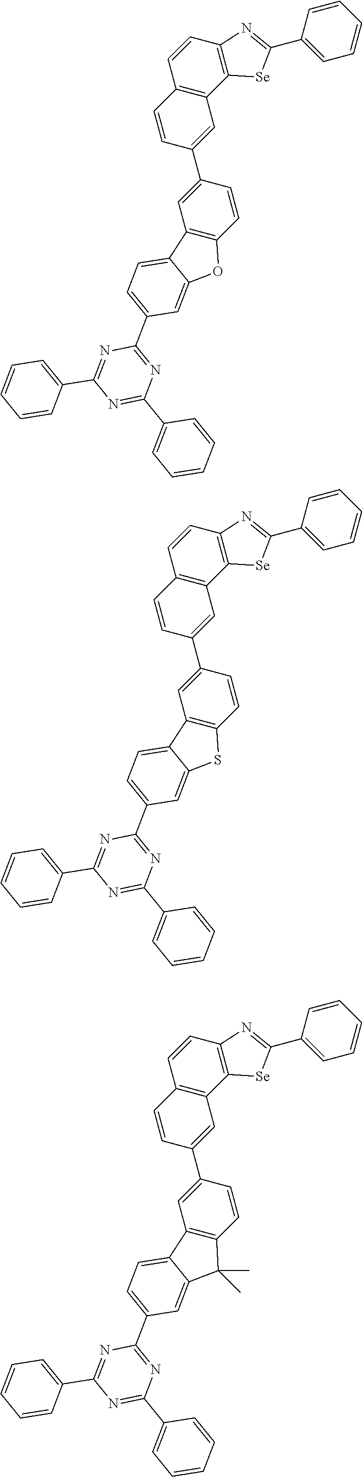 [SMILES: c1ccc(-c2nc(-c3ccccc3)nc(-c3ccc4c(c3)oc3ccc(-c5ccc6ccc7nc(-c8ccccc8)[se]c7c6c5)cc34)n2)cc1 ; c1ccc(-c2nc(-c3ccccc3)nc(-c3ccc4c(c3)sc3ccc(-c5ccc6ccc7nc(-c8ccccc8)[se]c7c6c5)cc34)n2)cc1 ; CC1(C)c2ccc(-c3ccc4ccc5nc(-c6ccccc6)[se]c5c4c3)cc2-c2ccc(-c3nc(-c4ccccc4)nc(-c4ccccc4)n3)cc21]

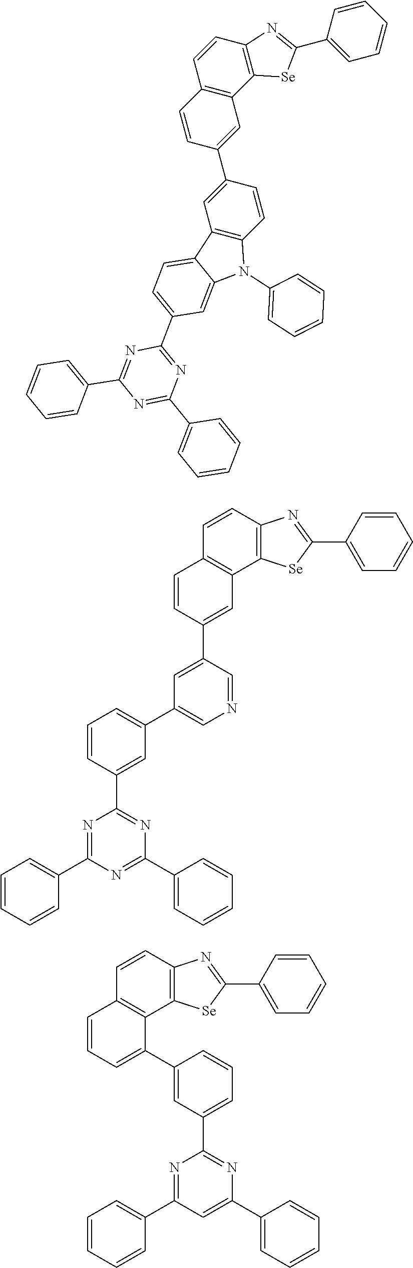

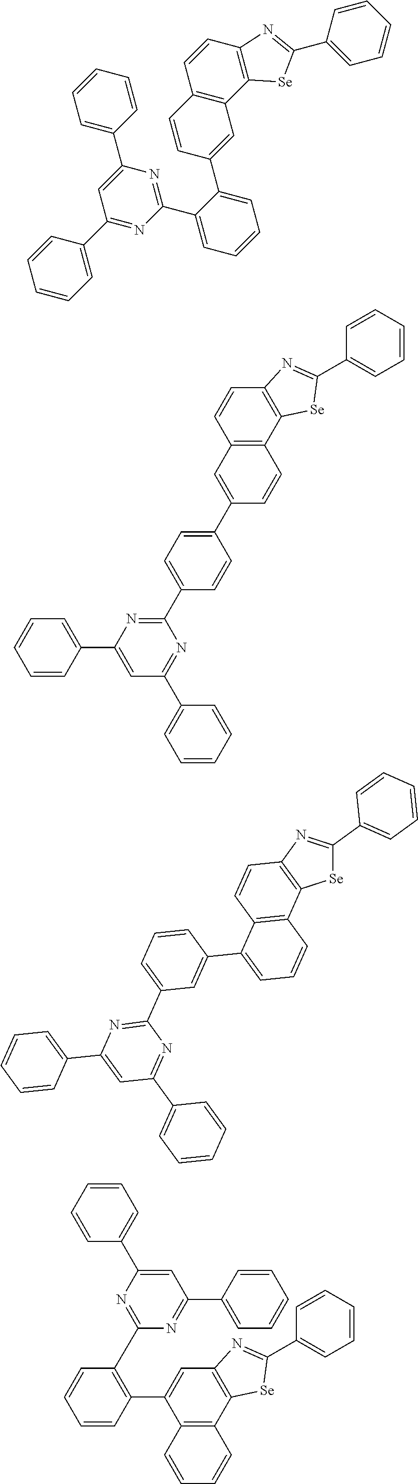 [SMILES: c1ccc(-c2cc(-c3ccccc3)nc(-c3ccccc3-c3ccc4ccc5nc(-c6ccccc6)[se]c5c4c3)n2)cc1 ; c1ccc(-c2cc(-c3ccccc3)nc(-c3ccc(-c4ccc5c(ccc6nc(-c7ccccc7)[se]c65)c4)cc3)n2)cc1 ; c1ccc(-c2cc(-c3ccccc3)nc(-c3cccc(-c4cccc5c4ccc4nc(-c6ccccc6)[se]c45)c3)n2)cc1 ; c1ccc(-c2cc(-c3ccccc3)nc(-c3ccccc3-c3cc4nc(-c5ccccc5)[se]c4c4ccccc34)n2)cc1]

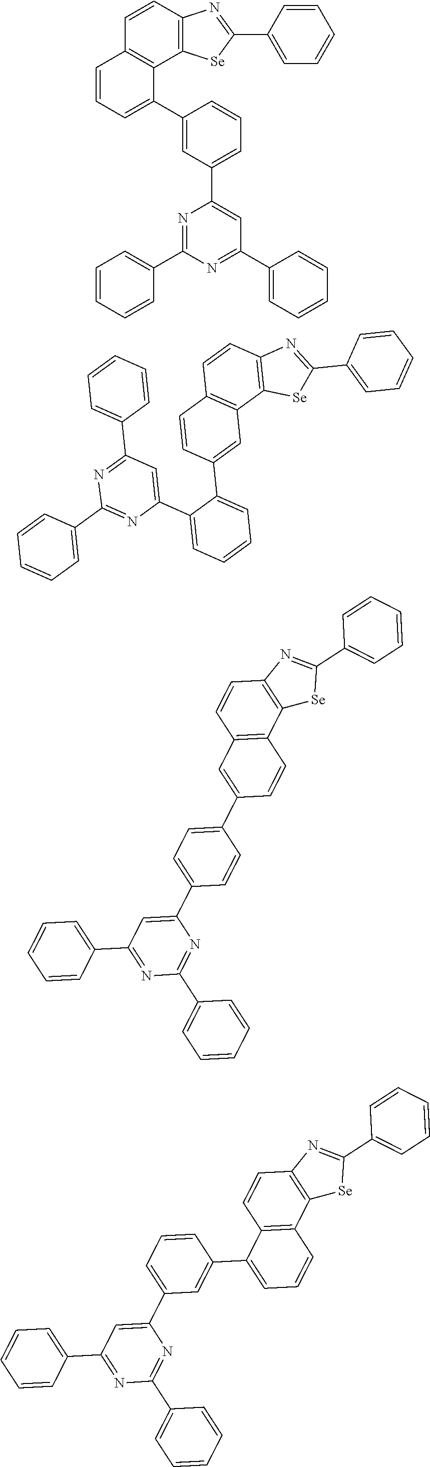

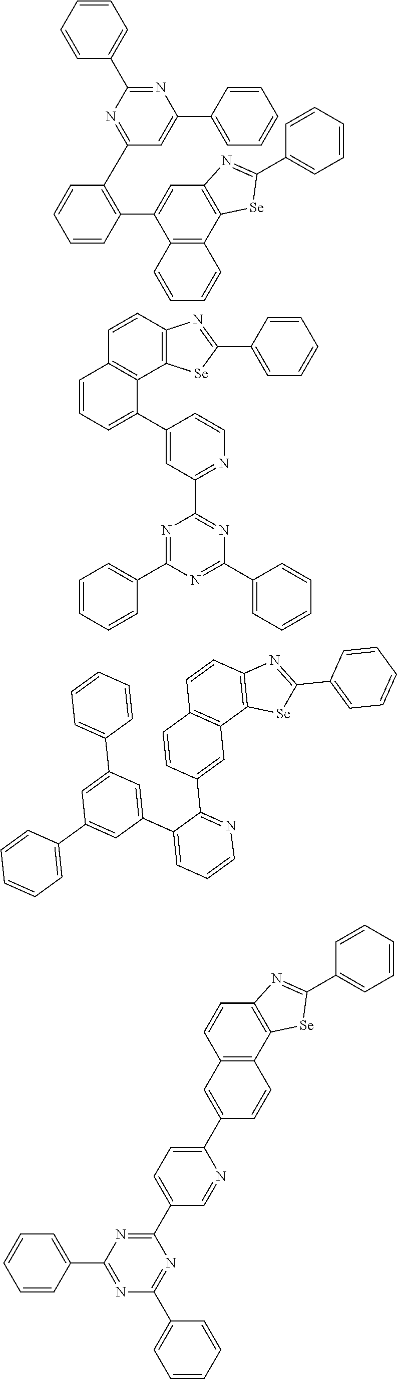

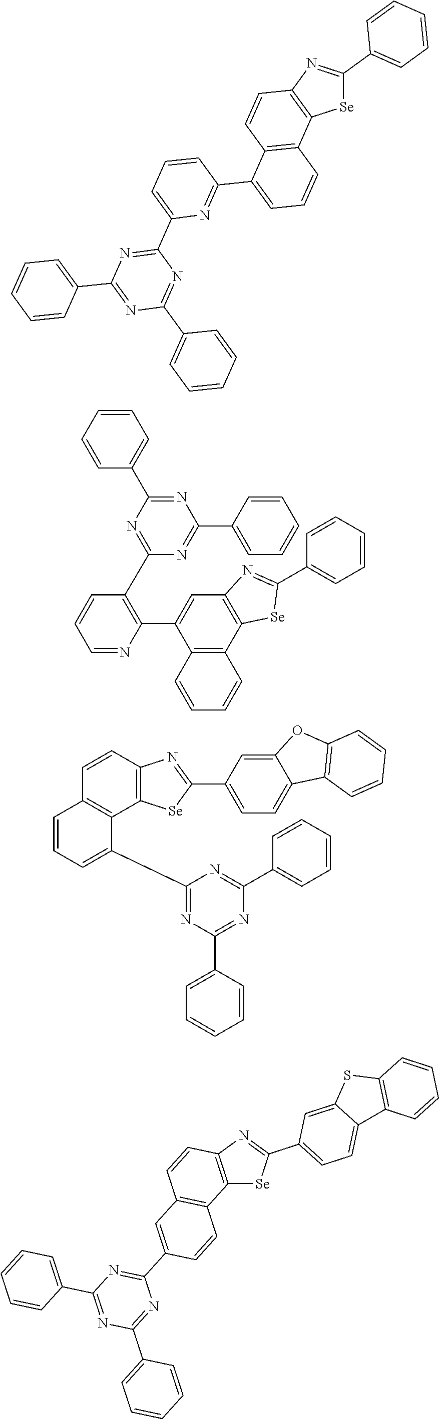 [SMILES: c1ccc(-c2nc(-c3ccccc3)nc(-c3cccc(-c4cccc5c4ccc4nc(-c6ccccc6)[se]c45)n3)n2)cc1 ; c1ccc(-c2nc(-c3ccccc3)nc(-c3cccnc3-c3cc4nc(-c5ccccc5)[se]c4c4ccccc34)n2)cc1 ; c1ccc(-c2nc(-c3ccccc3)nc(-c3cccc4ccc5nc(-c6ccc7c(c6)oc6ccccc67)[se]c5c34)n2)cc1 ; c1ccc(-c2nc(-c3ccccc3)nc(-c3ccc4c(ccc5nc(-c6ccc7c(c6)sc6ccccc67)[se]c54)c3)n2)cc1]

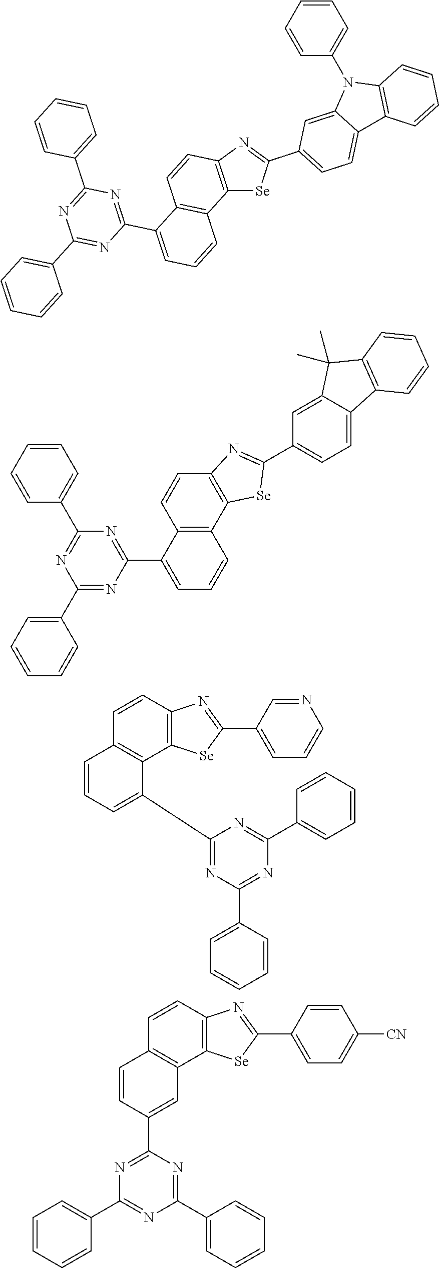

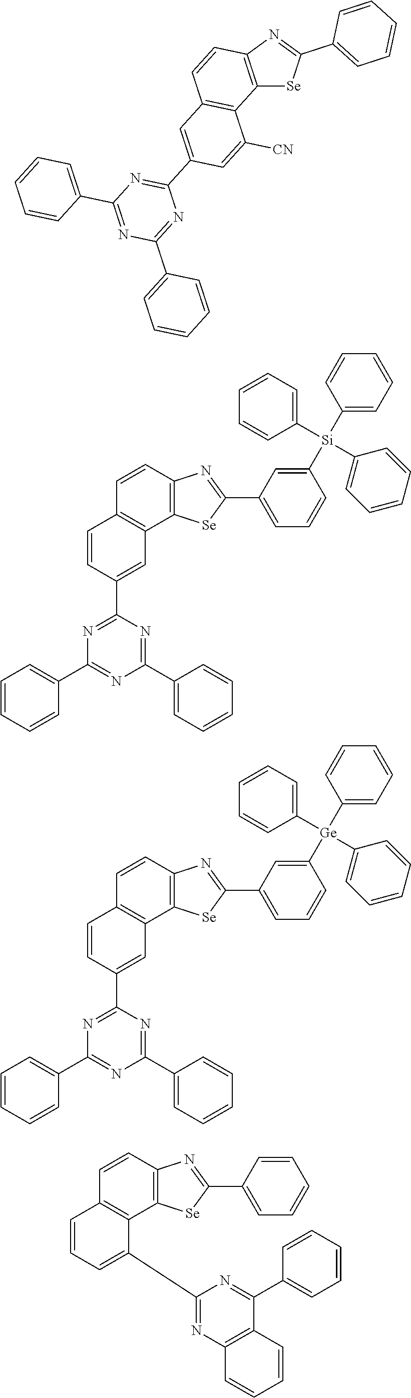

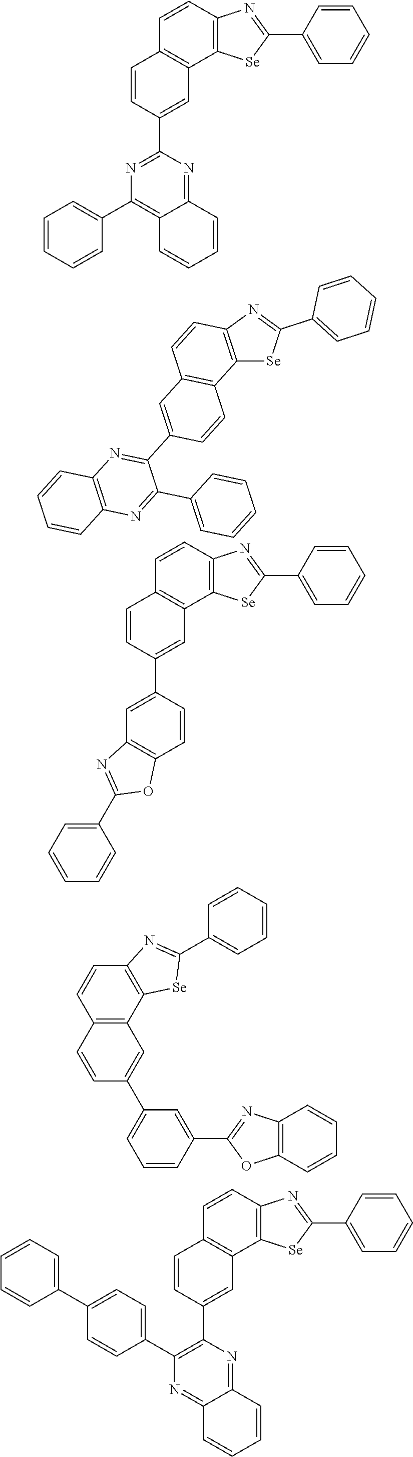

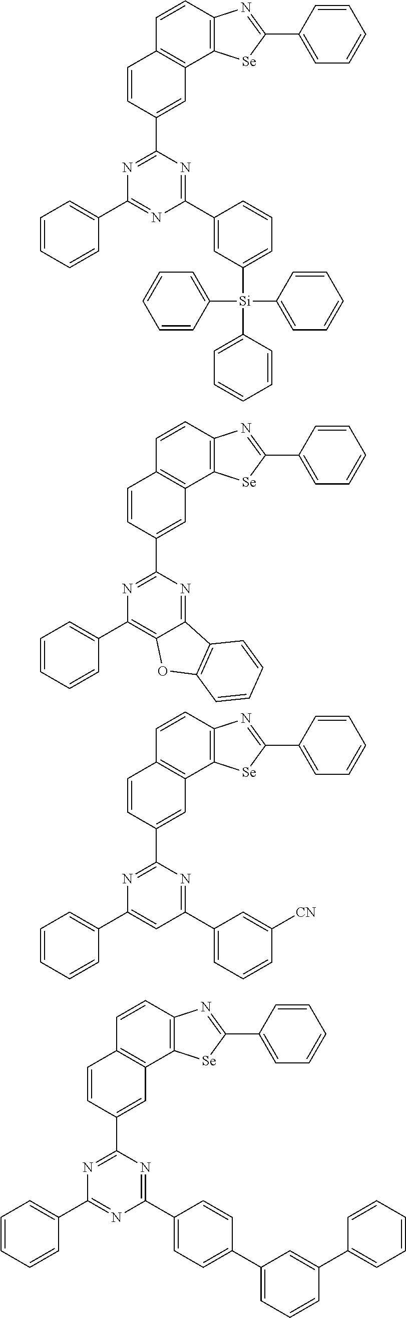

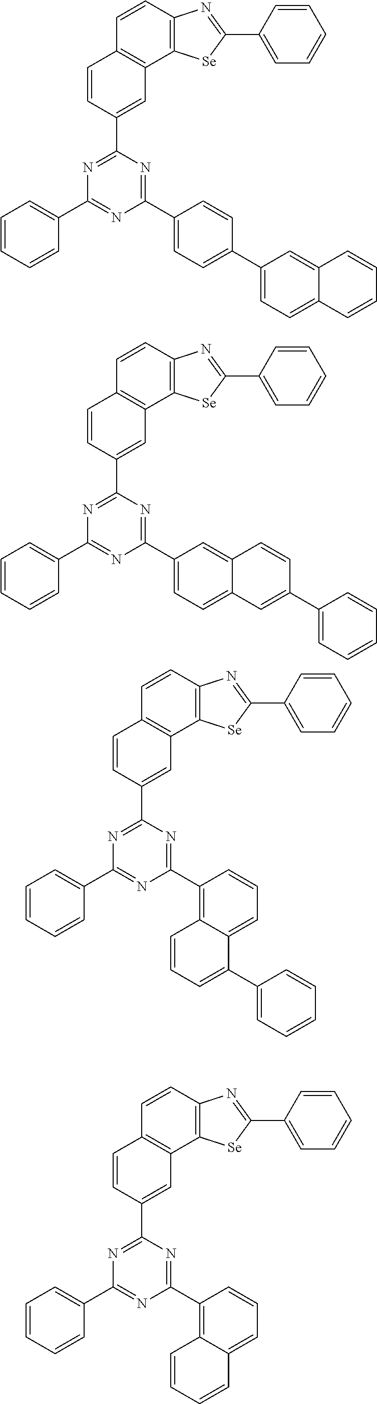

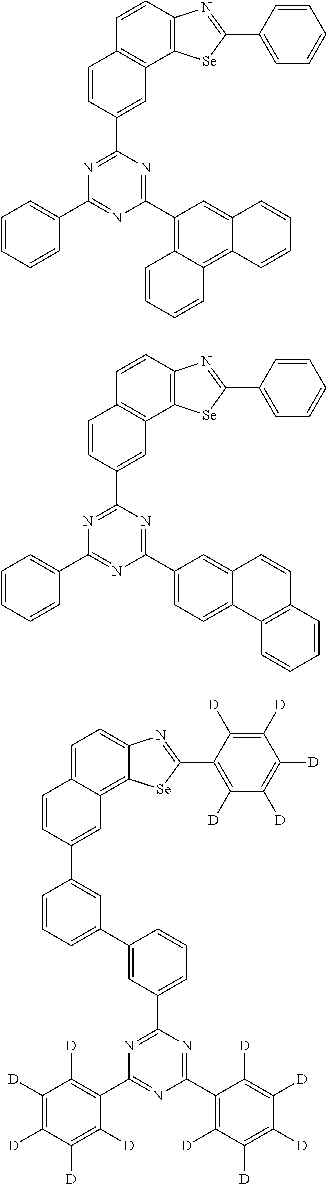

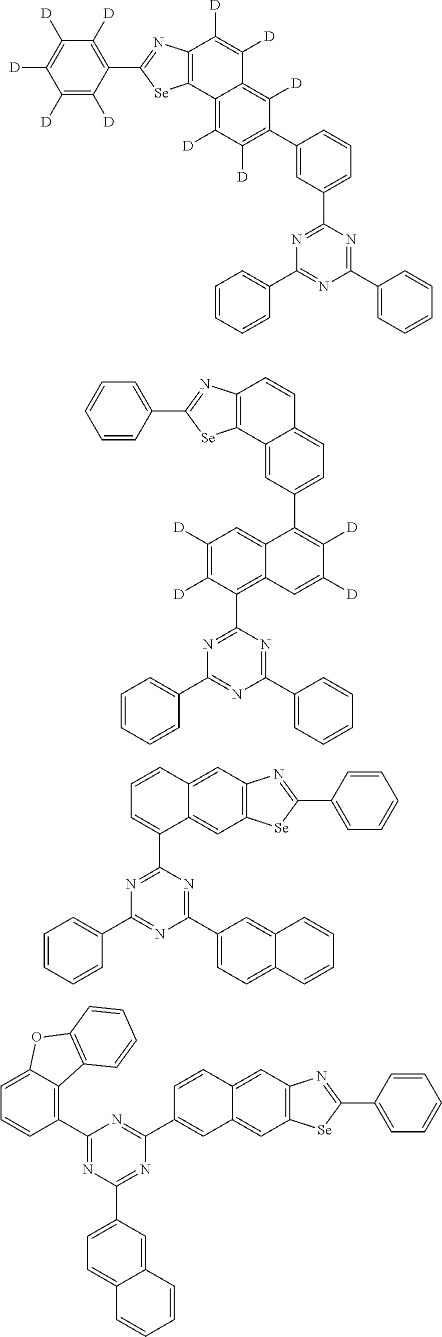

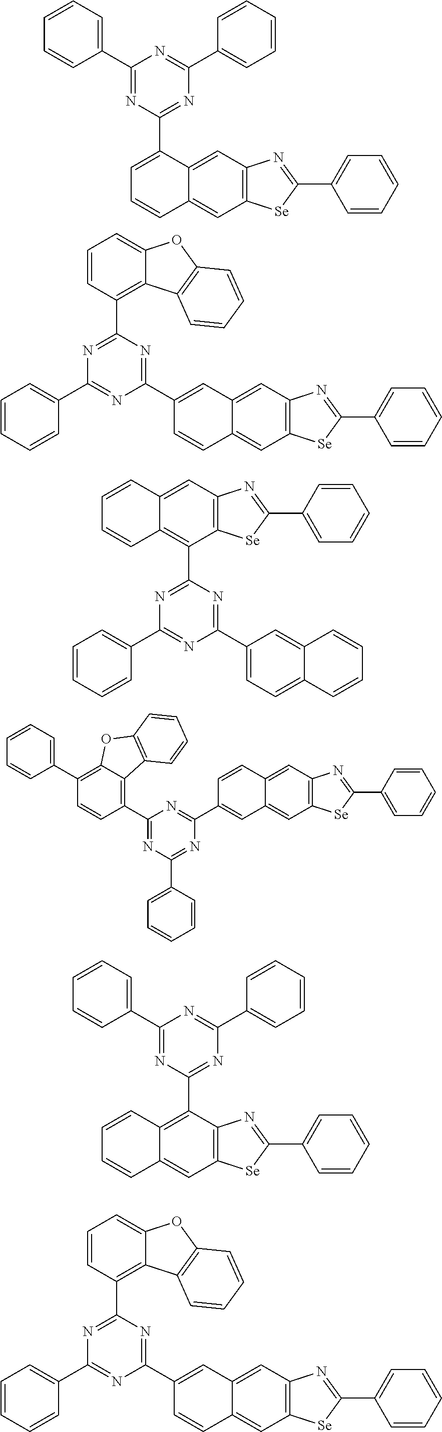

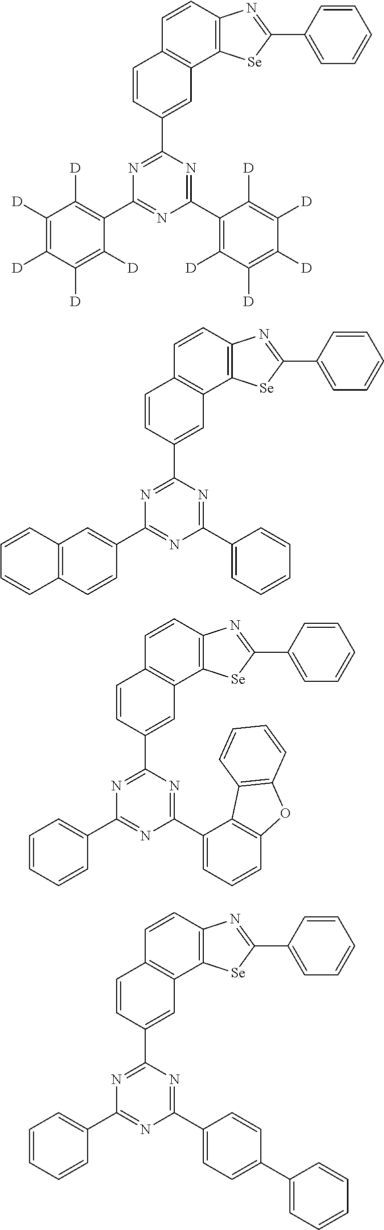

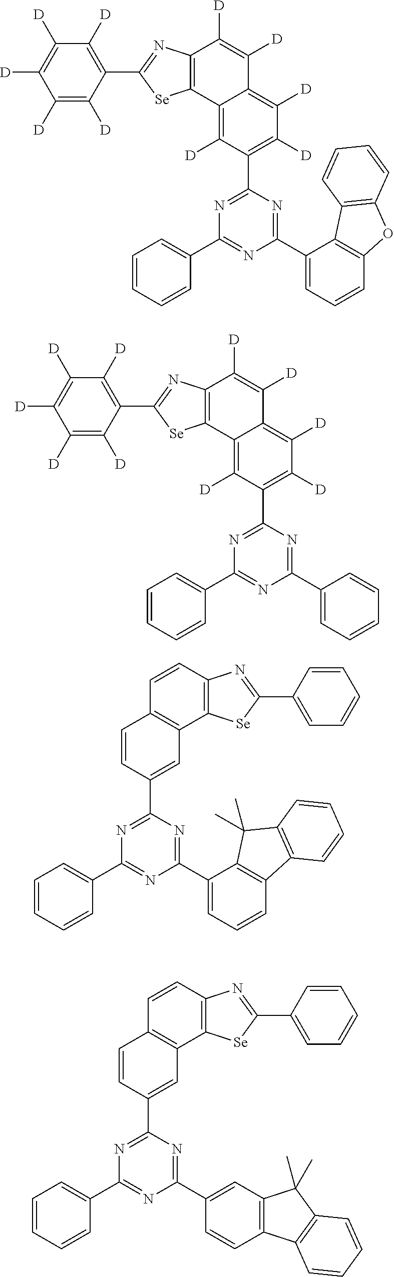

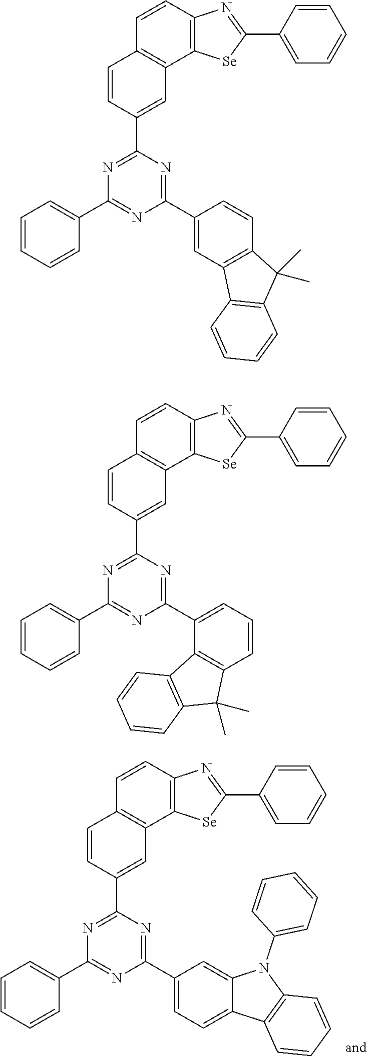

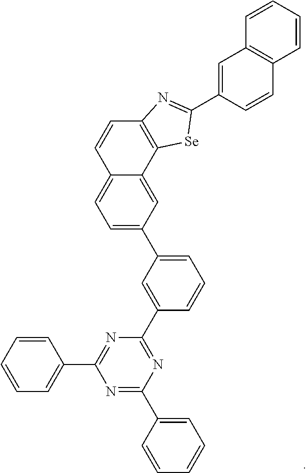 [SMILES: c1ccc(-c2nc(-c3ccccc3)nc(-c3cccc(-c4ccc5ccc6nc(-c7ccc8ccccc8c7)[se]c6c5c4)c3)n2)cc1]

13. An organic electroluminescent device comprising the organic electroluminescent compound according to claim 1.
